(19) United States
(12) Patent Application Publication
(10) Pub. No.: US 2023/0131493 A1
(43) Pub. Date: Apr. 27, 2023

(54) METHODS OF TREATING AGITATION ASSOCIATED WITH ALZHEIMER'S DISEASE

(71) Applicant: Avanir Phrmaceuticals Inc., Aliso Viejo, CA (US)

(72) Inventor: Sanjay Dube, Aliso Viejo, CA (US)

(21) Appl. No.: 17/974,132

(22) Filed: Oct. 26, 2022

Related U.S. Application Data
(60) Provisional application No. 63/275,091, filed on Nov. 3, 2021.
(60) Provisional application No. 63/272,471, filed on Oct. 27, 2021.

(51) Int. Cl. A61K 31/485; A61K 31/4709; A61P 25/28

(52) U.S. Cl. CPC A61K 31/485; A61K 31/4709; A61P 25/28

(57) ABSTRACT

In some embodiments provided herein is a method of treating agitation associated with Alzheimer's disease in a subject, comprising administering to the subject therapeutically effective amounts of deuterated [d6]-dextromethorphan hydrobromide (d6-DM) and quinidine sulfate, wherein the subject is a patient that has been diagnosed as having Alzheimer's disease, wherein the method comprises determining the Cohen-Mansfield agitation inventory (CMAI) total score in the patient prior to said administration.

The present disclosure relates to the treatment of agitation associated with Alzheimer's disease. In some embodiments the present disclosure provides methods of treating agitation associated with Alzheimer's disease using deuterated [d6]-dextromethorphan or a salt thereof and quinidine or a salt thereof. In some embodiments the present disclosure provides methods of treating agitation associated with Alzheimer's disease using deuterated [d6]-dextromethorphan hydrobromide (d6-DM) and quinidine sulfate.

### BACKGROUND

Alzheimer's disease, the most common form of dementia, is a progressive neurodegenerative disease that eventually leads to death. There are an estimated 5.8 million people in the United States (US) with Alzheimer's dementia and this number is expected to reach 14 million by the year 2050. National estimates of the prevalence of all dementias from population-based studies including the Aging, Demographics, and Memory Study (ADAMS), a nationally representative sample of older adults, show that 14 percent of people age 71 and older in the US have dementia.

Agitation is widely recognized by clinicians as a common and important clinical feature of Alzheimer's disease and other forms of dementia. Agitation, aggression, depression, hallucinations, and delusions are estimated to affect up to approximately 90% of patients with Alzheimer's disease with an increase in prevalence as the disease progresses. In a meta analyses of data from 55 studies, overall prevalence of agitation ranged from 5% to 88% across all studies, with 23 studies reporting the prevalence of at least one neuropsychiatric syndrome with a range of 40% to 100%. Agitation in patients with dementia is associated with increased functional disability, worse quality of life, earlier institutionalization, increased caregiver burden, increased healthcare costs, shorter time to severe dementia, and accelerated mortality. Currently, there is no approved treatment in the US to manage agitation in patients with Alzheimer's disease. Pharmacologic treatments for patients with agitation in Alzheimer's disease include off-label use of atypical antipsychotics, selective serotonin reuptake inhibitors, benzodiazepines, and anticonvulsants. It is widely recognized that a safe and effective treatment for patients with agitation in Alzheimer's disease represents a significant unmet need. Such a treatment could profoundly impact patient care, potentially reduce caregiver burden, and improve overall disease prognosis.

### SUMMARY

This disclosure provides, in some embodiments, methods of treating agitation associated with Alzheimer's disease in a subject, comprising administering to the subject therapeutically effective amounts of deuterated [d6]-dextromethorphan, or a salt thereof, and quinidine, or a salt thereof. In some embodiments the salt of deuterated [d6]-dextromethorphan is a deuterated [d6]-dextromethorphan hydrobromide, In some embodiments the deuterated [d6]-dextromethorphan hydrobromide is a deuterated [d6]-dextromethorphan hydrobromide hydrate, such as [d6]-dextromethorphan hydrobromide monohydrate. In some embodiments, provided herein are methods of treating agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments the salt of deuterated [d6]-dextromethorphan is a deuterated [d6]-dextromethorphan hydrobromide, In some embodiments the deuterated [d6]-dextromethorphan hydrobromide is a deuterated [d6]-dextromethorphan hydrobromide hydrate, such as [d6]-dextromethorphan hydrobromide monohydrate. In some embodiments, provided herein are methods of treating agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating agitation associated with Alzheimer's disease in a patient having Alzheimer's disease, comprising administering to the patient therapeutically effective amounts of d6-DM and quinidine sulfate.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the Cohen-Mansfield agitation inventory (CMAI) total score in the patient prior to said administration.

In some embodiments, provided herein is a composition comprising deuterated [d6]-dextromethorphan hydrobromide (d6-DM) and quinidine sulfate, for use in the treatment of agitation associated with Alzheimer's disease in a subject, in administering to the subject therapeutically effective amounts of deuterated [d6]-dextromethorphan hydrobromide (d6-DM) and quinidine sulfate, wherein the subject is a patient that has been diagnosed as having Alzheimer's disease, wherein the method comprises determining the Cohen-Mansfield agitation inventory (CMAI) total score in the patient prior to said administration. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, provided herein is a composition comprising deuterated [d6]-dextromethorphan hydrobromide (d6-DM) and quinidine sulfate, for use in the treatment of agitation associated with Alzheimer's disease in a subject, wherein the subject is a patient that has been diagnosed as having Alzheimer's disease, wherein the method comprises determining the Cohen-Mansfield agitation inventory (CMAI) total score in the patient prior to said administration. Additionally, the present disclosure also provides use of the composition comprising deuterated [d6]-dextromethorphan hydrobromide (d6-DM) and quinidine sulfate, in the manufacture of a medicament, wherein the composition is to be administered in the treatment of agitation associated with Alzheimer's disease in a subject, wherein the subject is a patient that has been diagnosed as having Alzheimer's disease, wherein the method comprises determining the Cohen-Mansfield agitation inventory (CMAI) total score in the patient prior to said administration. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 32, such as 33 to 203, such as 45 to 120, such as 55 to 90.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the Cohen-Mansfield agitation inventory (CMAI) total score in the patient prior to said administration.

In some embodiments, provided herein is a composition comprising deuterated [d6]-dextromethorphan hydrobromide (d6-DM) and quinidine sulfate, for use in the treatment of agitation associated with Alzheimer's disease in a subject, in administering to the subject therapeutically effective amounts of deuterated [d6]-dextromethorphan hydrobromide (d6-DM) and quinidine sulfate, wherein the subject is a patient that has been diagnosed as having Alzheimer's disease, wherein the method comprises determining the Cohen-Mansfield agitation inventory (CMAI) total score in the patient prior to said administration. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, provided herein is a composition comprising deuterated [d6]-dextromethorphan hydrobromide (d6-DM) and quinidine sulfate, for use in the treatment of agitation associated with Alzheimer's disease in a subject, wherein the subject is a patient that has been diagnosed as having Alzheimer's disease, wherein the method comprises determining the Cohen-Mansfield agitation inventory (CMAI) total score in the patient prior to said administration. Additionally, the present disclosure also provides use of the composition comprising deuterated [d6]-dextromethorphan hydrobromide (d6-DM) and quinidine sulfate, in the manufacture of a medicament, wherein the composition is to be administered in the treatment of agitation associated with Alzheimer's disease in a subject, wherein the subject is a patient that has been diagnosed as having Alzheimer's disease, wherein the method comprises determining the Cohen-Mansfield agitation inventory (CMAI) total score in the patient prior to said administration. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 32, such as 33 to 150, such as 45 to 120, such as 55 to 90.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25.

This disclosure provides, in some embodiments, methods of treating aggressive agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating aggressive agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating aggressive agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating aggressive agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating aggressive agitation associated with Alzheimer's disease in a patient having Alzheimer's disease, comprising administering to the patient therapeutically effective amounts of d6-DM and quinidine sulfate.

This disclosure provides, in some embodiments, methods of treating physically nonaggressive agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating physically nonaggressive agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating physically nonaggressive agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating physically nonaggressive agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating physically nonaggressive agitation associated with Alzheimer's disease in a patient having Alzheimer's disease, comprising administering to the patient therapeutically effective amounts of d6-DM and quinidine sulfate.

This disclosure provides, in some embodiments, methods of treating verbal agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating verbal agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating verbal agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating verbal agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating verbal agitation associated with Alzheimer's disease in a patient having Alzheimer's disease, comprising administering to the patient therapeutically effective amounts of d6-DM and quinidine sulfate.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI aggressive behavior score in the patient prior to said administration.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating aggressive agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI aggressive behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI aggressive behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19, 20, 21, 22, 23, 24, 25, 26, 27, 28, 29, 30, 31, 32, 33, 34, 35, 36, 37, 38, 39 or 40.

In some more particular embodiments, the patient has been assessed as having a CMAI aggressive behavior score of greater than or equal to 15.

In some more particular embodiments, the patient has been assessed as having a CMAI aggressive behavior score of greater than or equal to 16.

In some more particular embodiments, the patient has been assessed as having a CMAI aggressive behavior score of greater than or equal to 17.

In some more particular embodiments, the patient has been assessed as having a CMAI aggressive behavior score of greater than or equal to 18.

In some more particular embodiments, the patient has been assessed as having a CMAI aggressive behavior score of greater than or equal to 19.

In some more particular embodiments, the patient has been assessed as having a CMAI aggressive behavior score of greater than or equal to 20.

In some more particular embodiments, the patient has been assessed as having a CMAI aggressive behavior score of greater than or equal to 21.

In some more particular embodiments, the patient has been assessed as having a CMAI aggressive behavior score of greater than or equal to 22.

In some more particular embodiments, the patient has been assessed as having a CMAI aggressive behavior score of greater than or equal to 23.

In some more particular embodiments, the patient has been assessed as having a CMAI aggressive behavior score of greater than or equal to 24.

In some more particular embodiments, the patient has been assessed as having a CMAI aggressive behavior score of greater than or equal to 25.

In some more particular embodiments, the patient has been assessed as having a CMAI aggressive behavior score of greater than or equal to 26.

In some more particular embodiments, the patient has been assessed as having a CMAI aggressive behavior score of greater than or equal to 27.

In some more particular embodiments, the patient has been assessed as having a CMAI aggressive behavior score of greater than or equal to 28.

In some more particular embodiments, the patient has been assessed as having a CMAI aggressive behavior score of greater than or equal to 29.

In some more particular embodiments, the patient has been assessed as having a CMAI aggressive behavior score of greater than or equal to 30.

In some more particular embodiments, the patient has been assessed as having a CMAI aggressive behavior score of greater than or equal to 31.

In some more particular embodiments, the patient has been assessed as having a CMAI aggressive behavior score of greater than or equal to 32.

In some more particular embodiments, the patient has been assessed as having a CMAI aggressive behavior score of greater than or equal to 33.

In some more particular embodiments, the patient has been assessed as having a CMAI aggressive behavior score of greater than or equal to 34.

In some more particular embodiments, the patient has been assessed as having a CMAI aggressive behavior score of greater than or equal to 35.

In some more particular embodiments, the patient has been assessed as having a CMAI aggressive behavior score of greater than or equal to 36.

In some more particular embodiments, the patient has been assessed as having a CMAI aggressive behavior score of greater than or equal to 37.

In some more particular embodiments, the patient has been assessed as having a CMAI aggressive behavior score of greater than or equal to 38.

In some more particular embodiments, the patient has been assessed as having a CMAI aggressive behavior score of greater than or equal to 39.

In some more particular embodiments, the patient has been assessed as having a CMAI aggressive behavior score of greater than or equal to 40.

In some embodiments of a method disclosed herein, the method comprises determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has
   a. at least one aggressive behavior occurring at least three times per week;
   b. at least two aggressive behaviors occurring at least one time per week;
   c. at least three aggressive behaviors occurring, at a rate of less than once per week; and/or
   d. at least two aggressive behaviors occurring, less than once per week, and at least one aggressive behavior occurring at least once per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least one aggressive behavior occurring at least three times per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least two aggressive behaviors occurring at least one time per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least three aggressive behaviors occurring, at a rate of less than once per week;
3) administering to the patient therapeutically effective amounts of d6-DM and quinidine sulfate;
   and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least two aggressive behaviors occurring, less than once per week, and at least one aggressive behavior occurring at least once per week;
3) administering to the patient therapeutically effective amounts of d6-DM and quinidine sulfate;
   and
4) determining the CMAI total score in the patient following the administering step.

In some embodiments, the difference between the CMAI total score in the patient prior to the administering step and the CMAI total score in the patient following the administering step is from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the CMAI total score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% c lower, such as 15% lower, than the CMAI total score in the patient prior to the administering step.

In some embodiments of a method disclosed herein, the method comprises determining the CMAI aggressive behavior score in the patient following the administering step.

In some embodiments, the difference between the CMAI aggressive behavior score in the patient prior to the administering step and the CMAI aggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI aggressive behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI aggressive behavior score in the patient prior to the administering step.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI physically nonaggressive behavior score in the patient prior to said administration.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating physically nonaggressive agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI physically nonaggressive behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI physically nonaggressive behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 1920, 21, 22, 23, 24, 25, 26, 27, 28, 29, or 30.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 1920, 21, 22, 23, 24, 25, 26, 27, 28, 29, or 30.

In some more particular embodiments, the patient has been assessed as having a CMAI physically nonaggressive behavior score of greater than or equal to 10.

In some more particular embodiments, the patient has been assessed as having a CMAI physically nonaggressive behavior score of greater than or equal to 11.

In some more particular embodiments, the patient has been assessed as having a CMAI physically nonaggressive behavior score of greater than or equal to 12.

In some more particular embodiments, the patient has been assessed as having a CMAI physically nonaggressive behavior score of greater than or equal to 13.

In some more particular embodiments, the patient has been assessed as having a CMAI physically nonaggressive behavior score of greater than or equal to 14.

In some more particular embodiments, the patient has been assessed as having a CMAI physically nonaggressive behavior score of greater than or equal to 15.

In some more particular embodiments, the patient has been assessed as having a CMAI physically nonaggressive behavior score of greater than or equal to 16.

In some more particular embodiments, the patient has been assessed as having a CMAI physically nonaggressive behavior score of greater than or equal to 17.

In some more particular embodiments, the patient has been assessed as having a CMAI physically nonaggressive behavior score of greater than or equal to 18.

In some more particular embodiments, the patient has been assessed as having a CMAI physically nonaggressive behavior score of greater than or equal to 19.

In some more particular embodiments, the patient has been assessed as having a CMAI physically nonaggressive behavior score of greater than or equal to 20.

In some more particular embodiments, the patient has been assessed as having a CMAI physically nonaggressive behavior score of greater than or equal to 21.

In some more particular embodiments, the patient has been assessed as having a CMAI physically nonaggressive behavior score of greater than or equal to 22.

In some more particular embodiments, the patient has been assessed as having a CMAI physically nonaggressive behavior score of greater than or equal to 23.

In some more particular embodiments, the patient has been assessed as having a CMAI physically nonaggressive behavior score of greater than or equal to 24.

In some more particular embodiments, the patient has been assessed as having a CMAI physically nonaggressive behavior score of greater than or equal to 25.

In some more particular embodiments, the patient has been assessed as having a CMAI physically nonaggressive behavior score of greater than or equal to 26.

In some more particular embodiments, the patient has been assessed as having a CMAI physically nonaggressive behavior score of greater than or equal to 27.

In some more particular embodiments, the patient has been assessed as having a CMAI physically nonaggressive behavior score of greater than or equal to 28.

In some more particular embodiments, the patient has been assessed as having a CMAI physically nonaggressive behavior score of greater than or equal to 29.

In some more particular embodiments, the patient has been assessed as having a CMAI physically nonaggressive behavior score of greater than or equal to 30.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has
   a. at least one physically nonaggressive behavior occurring once or twice a day;
   b. at least two physically nonaggressive behaviors occurring at least three times per week;
   c. at least three physically nonaggressive behaviors occurring, at least one time per week; and/or
   d. at least four physically nonaggressive behaviors occurring less than once per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least one physically nonaggressive behavior occurring once or twice a day;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least two physically nonaggressive behaviors occurring at least three times per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least three physically nonaggressive behaviors occurring at least one time per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least four physically nonaggressive behaviors occurring less than once per week;
3) administering to the patient therapeutically effective amounts of d6-DM and quinidine sulfate; and
4) determining the CMAI total score in the patient following the administering step.

In some embodiments of a method disclosed herein, the method comprises determining the CMAI physically nonaggressive behavior score in the patient following the administering step.

In some embodiments, the difference between the CMAI physically nonaggressive behavior score in the patient prior to the administering step and the CMAI physically nonaggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI physically nonaggressive behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI physically nonaggressive behavior score in the patient prior to the administering step.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the CMAI verbally agitated behavior score in the patient prior to said administration.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating verbal agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI verbally agitated behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI verbally agitated behavior score in the patient prior to said administration.

some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25.

In some more particular embodiments, the patient has been assessed as having a CMAI verbally agitated behavior score of greater than or equal to 8.

In some more particular embodiments, the patient has been assessed as having a CMAI verbally agitated behavior score of greater than or equal to 9.

In some more particular embodiments, the patient has been assessed as having a CMAI verbally agitated behavior score of greater than or equal to 10.

In some more particular embodiments, the patient has been assessed as having a CMAI verbally agitated behavior score of greater than or equal to 11.

In some more particular embodiments, the patient has been assessed as having a CMAI verbally agitated behavior score of greater than or equal to 12.

In some more particular embodiments, the patient has been assessed as having a CMAI verbally agitated behavior score of greater than or equal to 13.

In some more particular embodiments, the patient has been assessed as having a CMAI verbally agitated behavior score of greater than or equal to 14.

In some more particular embodiments, the patient has been assessed as having a CMAI verbally agitated behavior score of greater than or equal to 15.

In some more particular embodiments, the patient has been assessed as having a CMAI verbally agitated behavior score of greater than or equal to 16.

In some more particular embodiments, the patient has been assessed as having a CMAI verbally agitated behavior score of greater than or equal to 17.

In some more particular embodiments, the patient has been assessed as having a CMAI verbally agitated behavior score of greater than or equal to 18.

In some more particular embodiments, the patient has been assessed as having a CMAI verbally agitated behavior score of greater than or equal to 19.

In some more particular embodiments, the patient has been assessed as having a CMAI verbally agitated behavior score of greater than or equal to 20.

In some more particular embodiments, the patient has been assessed as having a CMAI verbally agitated behavior score of greater than or equal to 21.

In some more particular embodiments, the patient has been assessed as having a CMAI verbally agitated behavior score of greater than or equal to 22.

In some more particular embodiments, the patient has been assessed as having a CMAI verbally agitated behavior score of greater than or equal to 23.

In some more particular embodiments, the patient has been assessed as having a CMAI verbally agitated behavior score of greater than or equal to 24.

In some more particular embodiments, the patient has been assessed as having a CMAI verbally agitated behavior score of greater than or equal to 25.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has
   a. at least one verbally agitated behavior occurring once or twice a day;
   b. at least two verbally agitated behaviors occurring at least three times per week;
   c. at least three verbally agitated behaviors occurring, at least one time per week; and/or
   d. at least four verbally agitated behaviors occurring less than once per week;
3) administering to the patient therapeutically effective amounts of d6-DM and quinidine sulfate;
   and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least one verbally agitated behavior occurring once or twice a day;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least two verbally agitated behaviors occurring at least three times per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least three verbally agitated behaviors occurring at least one time per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step:
2) determining that the patient prior to the administering step has at least four verbally agitated behaviors occurring less than once per week;
3) administering to the patient therapeutically effective amounts of d6-DM and quinidine sulfate; and
4) determining the CMAI total score in the patient following the administering step.

In some embodiments of a method disclosed herein, the method comprises determining the CMAI verbally agitated behavior score in the patient following the administering step.

In some embodiments, the difference between the CMAI verbally agitated behavior score in the patient prior to the administering step and the CMAI verbally agitated behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI verbally agitated behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI verbally agitated behavior score in the patient prior to the administering step.

In some embodiments provided herein is a method of treating agitation associated with Alzheimer's disease in a patient that has been diagnosed as having Alzheimer's disease,

wherein the patient is a male,
wherein the method comprises:
a) determining the Cohen-Mansfield agitation inventory (CMAI) total score in the patient;
and
b) administering to the patient effective amounts of d6-DM and quinidine sulfate,

In some embodiments provided herein is a method of treating agitation associated with Alzheimer's disease in a patient that has been diagnosed as having Alzheimer's disease,

wherein the patient is a male,
wherein the method comprises:
a) determining the Cohen-Mansfield agitation inventory (CMAI) total score in the patient;
and
b) administering to the patient effective amounts of d6-DM and quinidine sulfate, wherein following the administering step, the CMAI total score is less than the CMAI total score prior to the administering step in the patient, wherein the decrease in the CMAI total score is at least three points greater than a decrease in the CMAI total score in a female patient following administering of the same amounts of d6-DM and quinidine sulfate.

In some embodiments provided herein is a method of treating agitation associated with Alzheimer's disease in a patient that has been diagnosed as having Alzheimer's disease,

wherein the patient is 65 years old or older,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 70 years old or older,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is from 80 years old to 85 years old,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) total score in the patient is less than 50;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) total score in the patient is less than 65;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has been treated or is being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) total score in the patient;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) total score in the patient;
   and
b) administering to the patient effective amounts of d6-DM and quinidine sulfate,
   wherein following the administration of the amounts, the CMAI total score is less than the CMAI total score prior to the administering step in the patient, wherein the decrease in the CMAI total score is greater, such as at least three points greater, than a decrease in the CMAI total score in a patient who has been treated or is being treated with an SSRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) total score in the patient;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) total score in the patient;
   and
b) administering to the patient effective amounts of d6-DM and quinidine sulfate,
   wherein following the administration of the amounts, the CMAI total score is less than the CMAI total score prior to the administering step in the patient, wherein the decrease in the CMAI total score is greater, such as at least three points greater, than a decrease in the CMAI total score in a patient who has been treated or is being treated with an SNRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 17 to 24,
wherein the method comprises:
a) determining the Cohen-Mansfield agitation inventory (CMAI) total score in the patient;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has an NPI-AA score >6,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is a male,
wherein the method comprises:
a) determining the Cohen-Mansfield agitation inventory (CMAI) total score in the patient;
and
b) administering to the patient effective amounts of d6-DM and quinidine sulfate,

In some embodiments provided herein is a method of treating agitation associated with Alzheimer's disease in a patient that has been diagnosed as having Alzheimer's disease,

wherein the patient is a male,
wherein the method comprises:
a) determining the Cohen-Mansfield agitation inventory (CMAI) total score in the patient;
and
b) administering to the patient effective amounts of d6-DM and quinidine sulfate, wherein following the administering step, the CMAI total score is less than the CMAI total score prior to the administering step in the patient, wherein the decrease in the CMAI total score is at least three points greater than a decrease in the CMAI total score in a female patient following administering of the same amounts of d6-DM and quinidine sulfate.

In some embodiments provided herein is a method of treating agitation associated with Alzheimer's disease in a patient that has been diagnosed as having Alzheimer's disease,

wherein the patient is greater or equal to 65 years old,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 75 years old or less,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 80 years old or older,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with an antipsychotic,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has not been treated and is not being treated with an antipsychotic following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has not been treated and is not being treated with memantime following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with a benzodiazepine,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) total score in the patient;
and
b) administering to the patient effective amounts of d6-DM and quinidine sulfate.
wherein following the administration of the amounts, the CMAI total score is less than the CMAI total score prior to the administering step in the patient, wherein the decrease in the CMAI total score is greater, such as at least three points greater, than a decrease in the CMAI total score in a patient who has not been treated and is not being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 13 to 19,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 65 years old or older,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 70 years old or older,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is from 80 years old to 85 years old,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is less than 50;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is less than 65;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with an SSRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
   and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with an SNRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 17 to 24,
wherein the method comprises:
a) determining the Cohen-Mansfield agitation inventory (CMAI) aggressive behavior score in the patient;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has an NPI-AA score >6,
wherein the method comprises:
a) determining the Cohen-Mansfield agitation inventory (CMAI) aggressive behavior score in the patient;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is greater or equal to 65 years old,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 75 years old or less,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 80 years old or older,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with an antipsychotic,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) aggressive behavior score in the patient:
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with an antipsychotic following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has not been treated and is not being treated with memantine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.
wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has not been treated and is not being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 13 to 19,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.
   wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a second patient following administering of the same amounts of d6-DM and quinidine sulfate to the second patient, wherein the method comprises determining that the Cohen-Mansfield agitation inventory (CMAI) aggressive behavior score in the second patient is less than 15 prior to the administering of the same amounts.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.
   wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a second patient following administering of the same amounts of d6-DM and quinidine sulfate to the second patient, wherein the method comprises determining that the Cohen-Mansfield agitation inventory (CMAI) aggressive behavior score in the second patient is less than 15 prior to the administering of the same amounts.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.
   wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a second patient following administering of the same amounts of d6-DM and quinidine sulfate to the second patient, wherein the method comprises determining that the Cohen-Mansfield agitation inventory (CMAI) aggressive behavior score in the second patient is less than 15 prior to the administering of the same amounts.

In some more particular embodiments, the amount of d6-DM is 42.63 mg.

In some more particular embodiments, the amount of d6-DM is 28 mg.

In some more particular embodiments, the amount of d6-DM is 18 mg.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with an SSRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with an SNRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with an antipsychotic following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has not been treated and is not being treated with memantine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.
wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has not been treated and is not being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with an SSRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
   and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with an SNRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with an antipsychotic following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has not been treated and is not being treated with memantine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.
wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has not been treated and is not being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments, the patient has been assessed as having a CMAI aggressive behavior score prior to administration of greater than or equal to 15.

In some embodiments, the patient has been assessed as having a CMAI aggressive behavior score prior to administration of greater than 15.

In some embodiments, the patient has been assessed as having a CMAI aggressive behavior score prior to administration of greater than 16.

In some embodiments, the patient has been assessed as having a CMAI aggressive behavior score prior to administration of greater than 17.

In some embodiments, following the administering step, the CMAI total score is lower than, such as at least 3 points lower than, the CMAI total score in the patient prior to the administering step.

In some embodiments, following the administering step, the CMAI total score is lower than, such as at least 4 points lower than, the CMAI total score in the patient prior to the administering step.

In some embodiments, following the administering step, the CMAI total score is lower than, such as at least 5 points lower than, the CMAI total score in the patient prior to the administering step.

In some more particular embodiments, the amount of d6-DM is 42.63 mg.

In some more particular embodiments, the amount of d6-DM is 28 mg.

In some more particular embodiments, the amount of d6-DM is 18 mg.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI physically nonaggressive behavior score that is the same as the CMAI physically nonaggressive behavior score prior to the administering step or that differs from the CMAI physically nonaggressive behavior score prior to the administering step by no more than 7, 6, 5, 4, 3, 2, or 1.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI physically nonaggressive behavior score that is the same or at least 1 less, 2 less, 3 less, 4 less, 5 less, 6 less, or 7 less than the CMAI physically nonaggressive behavior score prior to the administering step.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI verbally agitated behavior score that is the same as the CMAI verbally agitated behavior score prior to the administering step or that differs from the CMAI verbally agitated behavior score prior to the administering step by no more than 7, 6, 5, 4, 3, 2, or 1.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI verbally agitated behavior score that is the same or at least 1 less, 2 less, 3 less, 4 less, 5 less, 6 less, or 7 less than the CMAI verbally agitated behavior score prior to the administering step.

In some embodiments the deuterated [d6]-dextromethorphan hydrobromide (d6-DM) is a deuterated [d6]-dextromethorphan hydrobromide hydrate, such as [d6]-dextromethorphan hydrobromide monohydrate.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the NPI-AA score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI-AA score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI-AA score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI-AA score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10.

In some embodiments of a method disclosed herein, the method comprises determining the NPI-AA score in the patient prior to the administering step.

In some embodiments the NPI-AA score prior to the administering step is equal to or greater than 4.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the NPI Aberrant Motor Behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI Aberrant Motor Behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI Aberrant Motor Behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI Aberrant Motor Behavior score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Aberrant Motor Behavior score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Aberrant Motor Behavior domain score greater than or equal to 2, such as 2 to 12, such as 2, 3, 4, 5, 6, 7, 8, 9, or 10.

In some embodiments of a method disclosed herein, the method comprises determining the NPI Aberrant Motor Behavior domain score in the patient prior to the administering step.

In some embodiments the NPI Aberrant Motor Behavior domain score prior to the administering step is greater than or equal to 2, such as 2 to 12, such as 2, 3, 4, 5, 6, 7, 8, 9, or 10.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the NPI Irritability/Lability domain score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI Irritability/Lability domain score in the patient prior to said administration.\

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI Irritability/Lability domain score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI Irritability/Lability domain score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Irritability/Lability domain score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Irritability/Lability domain score greater than or equal to 2, such as 2 to 12, such as 2, 3, 4, 5, 6, 7, 8, 9, or 10.

In some embodiments of a method disclosed herein, the method comprises determining the NPI Irritability/Lability domain score in the patient prior to the administering step.

In some embodiments the NPI Irritability/Lability domain score prior to the administering step is equal to or greater than 2, such as 2 to 12, such as 2, 3, 4, 5, 6, 7, 8, 9, or 10.

In some embodiments of a method disclosed herein, the method comprises determining the NPI total score in the patient prior to the administering step.

In some embodiments the NPI total score prior to the administering step is equal to or greater than 1.

In some embodiments of a method disclosed herein, the method comprises determining the NPI-AA score in the patient following the administering step.

In some embodiments, the difference between the NPI-AA score in the patient prior to the administering step and the NPI-AA score in the patient following the administering step is at least 1, such as 1 to 9, such as 1, 2, 3, 4, 5, 6, or 7.

In some embodiments of a method disclosed herein, the method comprises determining the NPI Aberrant Motor Behavior domain score in the patient following the administering step.

In some embodiments, the difference between the NPI Aberrant Motor Behavior domain score in the patient prior to the administering step and the NPI Aberrant Motor Behavior domain score in the patient following the administering step is at least 1, such as 1 to 9, such as 1, 2, 3, 4, 5, 6, or 7.

In some embodiments of a method disclosed herein, the method comprises determining the NPI Irritability/Lability domain score in the patient following the administering step.

In some embodiments, the difference between the NPI Irritability/Lability domain score in the patient prior to the administering step and the NPI Irritability/Lability domain score in the patient following the administering step is at least 1, such as 1 to 9, such as 1, 2, 3, 4, 5, 6, or 7.

In some embodiments of a method disclosed herein, the method comprises determining the NPI total score in the patient following the administering step.

In some embodiments, the difference between the NPI total score in the patient prior to the administering step and the NPI total score in the patient following the administering step is at least 1, such as 1 to 25, such as 1, 2, 3, 4, 5, 6, 7, 8, 9, 10, 11, 12, 13, 14, 15, 16, 17, 18, 19, 20, 21, 22, 23, 24, or 25.

In some embodiments of a method disclosed herein, the method comprises determining the MMSE score in the patient prior to the administering step.

In some embodiments, the MMSE score prior to the administering step is 4 to 30.

In some embodiments the MMSE score prior to the administering step is from 8 to 24.

In some embodiments the MMSE score prior to the administering step is from 6 to 26.

In some embodiments the MMSE score prior to the administering step is equal to or greater than 17.

In some embodiments of a method disclosed herein, the method comprises determining the CGIS-Agitation score in the patient prior to the administering step.

In some embodiments of a method disclosed herein, the CGIS-Agitation score in the patient prior to the administering step is greater than or equal to 2.

In some embodiments of a method disclosed herein, the CGIS-Agitation score in the patient prior to the administering step is greater than or equal to 3.

In some embodiments of a method disclosed herein, the CGIS-Agitation score in the patient prior to the administering step is greater than or equal to 4.

In some embodiments of a method disclosed herein, the method comprises determining the mADCS-CGIC-Agitation score in the patient following the administering step.

In some more particular embodiments, the mADCS-CGIC-Agitation score is ≤2 following the administering step.

In some more particular embodiments, the mADCS-CGIC-Agitation score is ≤3 following the administering step.

In some more particular embodiments, the mADCS-CGIC-Agitation score is ≤4 following the administering step.

In some embodiments of a method disclosed herein, the method comprises determining the PGIC score in the patient following the administering step.

In some more particular embodiments, the PGIC score is ≤2 following the administering step.

In some more particular embodiments, the PGIC score is ≤3 following the administering step.

In some more particular embodiments, the PGIC score is ≤4 following the administering step.

In some embodiments of a method disclosed herein, the method comprises determining the CGIS-Agitation score in the patient following the administering step.

In some embodiments, the CGIS-Agitation score in the patient following the administering step is at least 15% lower, such as at least 30% lower, than the CGIS-Agitation score in the patient prior to the administering step.

An exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the d6-DM is administered in a dose of from 14.4 mg to 22.5 mg twice daily and the quinidine sulfate is administered in a dose of from 3.9 mg to 6.1 mg dose twice daily. An exemplary embodiment of the present disclosure includes a method of treating agitation associated with Alzheimer's disease in a patient, comprising administering to the patient therapeutically effective amounts of d6-DM and quinidine sulfate, wherein the d6-DM is administered in dose of from 34.4 mg to 53.8 mg twice daily twice daily and the quinidine sulfate is administered in a from 3.9 mg to 6.1 mg dose twice daily.

An exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the d6-DM is administered in a dose of from 14.4 mg to 22.5 mg twice daily and the quinidine sulfate is administered in a 4.9 mg dose twice daily. An exemplary embodiment of the present disclosure includes a method of treating agitation associated with Alzheimer's disease in a patient, comprising administering to the patient therapeutically effective amounts of d6-DM and quinidine sulfate, wherein the d6-DM is administered in dose of from 34.4 mg to 53.8 mg twice daily twice daily and the quinidine sulfate is administered in a 4.9 mg dose twice daily.

In some embodiments, the d6-DM is administered in an 18 mg dose twice daily and the quinidine sulfate is administered in a 4.9 mg dose twice daily. In some embodiments, the d6-DM is administered in a 42.63 mg dose twice daily and the quinidine sulfate is administered in a 4.9 mg dose twice daily.

In some embodiments of a method disclosed herein, the administration of one component (e.g., d6-DM) is concomitant with the administration of the other component (e.g., quinidine sulfate).

In some embodiments of a method disclosed herein, the patient has been treated or is being treated with memantine prior to the administering step.

In some embodiments of a method disclosed herein, the patient has been treated or is being treated with an acetylcholinesterase inhibitor, such as donepezil, prior to the administering step.

In some embodiments of a method disclosed herein, the patient has been treated or is being treated with an atypical antipsychotic other than clozapine prior to the administering step.

In some embodiments of a method disclosed herein, the patient has been treated or is being treated with an antidepressant other than nefazodone, a tricyclic antidepressant, or a monoamine oxidase inhibitors (MAOI) prior to the administering step.

In some embodiments of a method disclosed herein, the patient has been treated or is being treated with memantine, and the patient has not been treated and is not being treated with antipsychotics, prior to the administering step.

In some embodiments of a method disclosed herein, the patient has been treated or is being treated with an acetylcholinesterase inhibitor, such as donepezil, and the patient has not been treated and is not being treated with antipsychotics, prior to the administering step.

In some embodiments of a method disclosed herein, the patient has been treated or is being treated with memantine, and the patient has not been treated and is not being treated with an acetylcholinesterase inhibitor, such as donepezil, prior to the administering step.

In some embodiments of a method disclosed herein, the patient has been treated or is being treated with an acetylcholinesterase inhibitor, such as donepezil, and the patient has not been treated and is not being treated with memantine, prior to the administering step.

In some embodiments, the d6-DM is administered in a 14.4, mg, 18 mg, or 22.5 mg dose, e.g., once or twice daily, e.g., twice daily. In some embodiments, the d6-DM is administered in a 14.4 mg dose, e.g., once or twice daily, e.g., twice daily. In some embodiments, the d6-DM is administered in a 18 mg dose, e.g., once or twice daily, e.g., twice daily. In some embodiments, the d6-DM is administered in a 22.5 mg dose, e.g., once or twice daily, e.g., twice daily.

In some embodiments, the d6-DM is administered in a 34.4, mg, 42.63 mg, or 53.8 mg dose, e.g., once or twice daily, e.g., twice daily. In some embodiments, the d6-DM is administered in a 34.4 mg dose, e.g., once or twice daily, e.g., twice daily. In some embodiments, the d6-DM is administered in a 42.63 mg dose, e.g., once or twice daily, e.g., twice daily. In some embodiments, the d6-DM is administered in a 53.8 mg dose, e.g., once or twice daily, e.g., twice daily.

In some embodiments, the quinidine sulfate is administered in a4.9 mg dose, e.g., once or twice daily, e.g., twice daily.

The chemical structure of deuterated [d6]-dextromethorphan is as follows.

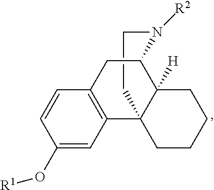

wherein:
R¹ is CD₃; and
R² CD₃.

The chemical structure of deuterated [d6]-dextromethorphan hydrobromide monohydrate is as follows.

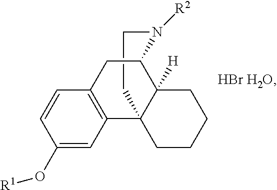

wherein:
R¹ is CD₃; and
R² CD₃.

The chemical structure of deuterated [d6]-dextromethorphan hydrobromide monohydrate is also shown at col. 11, lines 38-59 in U.S. Pat. No. 10,730,841 B2, incorporated by reference herein in its entirety.

In some embodiments, provided herein are methods of treating agitation associated with Alzheimer's disease in a subject as disclosed in PCT/US2021/029246, incorporated by reference herein in its entirety.

In some embodiments, provided herein are methods of treating agitation associated with Alzheimer's disease in a subject as disclosed in U.S. 63/016,270, incorporated by reference herein in its entirety.

### BRIEF DESCRIPTION OF THE DRAWINGS

FIG. 1 shows a Sequential Parallel Comparison Design Schematic for Example 3.

FIG. 2 shows a Study Schematic for the Study in Example 4.

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein, including, but not limited to, published and unpublished applications, patents, and literature references, are incorporated herein by reference in their entirety and are hereby made a part of this specification. To the extent a cited reference conflicts with the disclosure herein, the specification shall control.

As used herein, “d6-DM” refers to deuterated [d6]-dextromethorphan hydrobromide. In some embodiments the deuterated [d6]-dextromethorphan hydrobromide is a deuterated [d6]-dextromethorphan hydrobromide hydrate, such as [d6]-dextromethorphan hydrobromide monohydrate.

As used herein, “a salt thereof” is any non-toxic salt that, upon administration to a recipient, is capable of providing, either directly or indirectly, the corresponding free acid or base. Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, .beta.-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

Unless otherwise specified, reference to a medication such as a benzodiazepine, a selective serotonin reuptake inhibitor (SSRI), a serotonin norepinephrine reuptake inhibitor (SNRI), an antipsychotic, an antidepressant, an anticonvulsant, donepezil (ARICEPT®), memantine (NAMENDA®), and/or other medications administered to the patient together with, prior to, or subsequently to deuterated [d6]-dextromethorphan or a salt thereof, such as deuterated [d6]-dextromethorphan hydrobromide (d6-DM), and quinidine or a salt thereof, such as quinidine sulfate, is intended to encompass the free bases or free acids, as applicable, and the acid salts or base salts, as applicable, of such medications. In some embodiments the deuterated [d6]-dextromethorphan hydrobromide is a deuterated [d6]-dextromethorphan hydrobromide hydrate, such as [d6]-dextromethorphan hydrobromide monohydrate.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

As used herein, the term “treating” means improving, ameliorating, or retarding the onset, progress, severity, or frequency of one or more behaviors associated with agitation associated with Alzheimer's disease.

The term “therapeutically effective amounts of deuterated [d6]-dextromethorphan (d6-DM) hydrobromide and quinidine sulfate” refers to the amount of d6-DM and the amount of quinidine sulfate that are sufficient to treat agitation associated with Alzheimer's disease when administered in combination. As an example, the amount of d6-DM and the amount of quinidine sulfate may be sufficient to reduce the CMAI aggressive behavior score in the patient. As an example, the amount of d6-DM and the amount of quinidine sulfate may be sufficient to reduce the CMAI physically nonaggressive behavior score in the patient. As an example, the amount of d6-DM and the amount of quinidine sulfate may be sufficient to reduce the CMAI verbally agitated behavior score in the patient. As used herein, the term “combination” applied to d6-DM and quinidine sulfate means a single pharmaceutical composition (formulation) comprising both d6-DM and quinidine sulfate or two separate pharmaceutical compositions (formulations), each comprising d6-DM or quinidine sulfate, to be administered in combination. In some embodiments the deuterated [d6]-dextromethorphan hydrobromide is a deuterated [d6]-dextromethorphan hydrobromide hydrate, such as [d6]-dextromethorphan hydrobromide monohydrate.

Administered “in combination” or “co-administration,” as used herein, refers to administration of d6-DM and quinidine sulfate concomitantly in one composition, or concomitantly in different compositions, or sequentially in either order. For sequential administration to be considered administration “in combination” or “co-administration,” the d6-DM and the quinidine sulfate are administered separated by a time interval that permits the resultant beneficial effect for treating agitation associated with Alzheimer's disease in a patient.

The term “patient” or “subject” means a human. In some embodiments, the patient is a human that has been diagnosed as having Alzheimer's disease.

Unless otherwise specified, the doses described herein refer to the hydrobromide and sulfate salt forms of deuterated [d6]-dextromethorphan and quinidine, respectively. Based on such information, those skilled in the art can calculate corresponding dosages for the free-base forms of each active ingredient. A person of skill in the art can calculate the molecular weight for the salt of deuterated [d6]-dextromethorphan and the molecular weight for free base of deuterated [d6]-dextromethorphan and use the ratio to calculate appropriate dosages for the free base as well as for a salt. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating agitation associated with Alzheimer's disease in a patient having Alzheimer's disease, comprising administering to the patient therapeutically effective amounts of d6-DM and quinidine sulfate.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the Cohen-Mansfield agitation inventory (CMAI) total score in the patient prior to said administration.

In some embodiments, provided herein is a composition comprising deuterated [d6]-dextromethorphan hydrobromide (d6-DM) and quinidine sulfate, for use in the treatment of agitation associated with Alzheimer's disease in a subject, in administering to the subject therapeutically effective amounts of deuterated [d6]-dextromethorphan hydrobromide (d6-DM) and quinidine sulfate, wherein the subject is a patient that has been diagnosed as having Alzheimer's disease, wherein the method comprises determining the Cohen-Mansfield agitation inventory (CMAI) total score in the patient prior to said administration. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, provided herein is a composition comprising deuterated [d6]-dextromethorphan hydrobromide (d6-DM) and quinidine sulfate, for use in the treatment of agitation associated with Alzheimer's disease in a subject, wherein the subject is a patient that has been diagnosed as having Alzheimer's disease, wherein the method comprises determining the Cohen-Mansfield agitation inventory (CMAI) total score in the patient prior to said administration. Additionally, the present disclosure also provides use of the composition comprising deuterated [d6]-dextromethorphan hydrobromide (d6-DM) and quinidine sulfate, in the manufacture of a medicament, wherein the composition is to be administered in the treatment of agitation associated with Alzheimer's disease in a subject, wherein the subject is a patient that has been diagnosed as having Alzheimer's disease, wherein the method comprises determining the Cohen-Mansfield agitation inventory (CMAI) total score in the patient prior to said administration. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 32, such as 33 to 150, such as 45 to 120, such as 55 to 90.

This disclosure provides, in some embodiments, methods of treating aggressive agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating aggressive agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating aggressive agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating aggressive agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating aggressive agitation associated with Alzheimer's disease in a patient having Alzheimer's disease, comprising administering to the patient therapeutically effective amounts of d6-DM and quinidine sulfate.

This disclosure provides, in some embodiments, methods of treating physically nonaggressive agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating physically nonaggressive agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating physically nonaggressive agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating physically nonaggressive agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating physically nonaggressive agitation associated with Alzheimer's disease in a patient having Alzheimer's disease, comprising administering to the patient therapeutically effective amounts of d6-DM and quinidine sulfate.

This disclosure provides, in some embodiments, methods of treating verbal agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating verbal agitation associated with Alzheimer's disease in a subject, comprising administering to the subject therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating verbal agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating verbal agitation associated with Alzheimer's disease in a subject, comprising administering to the subject a therapeutically effective amount of a composition comprising deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate.

In some embodiments, the present disclosure provides a method of treating verbal agitation associated with Alzheimer's disease in a patient having Alzheimer's disease, comprising administering to the patient therapeutically effective amounts of d6-DM and quinidine sulfate.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI aggressive behavior score in the patient prior to said administration.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating aggressive agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI aggressive behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI aggressive behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40.

In some more particular embodiments, the patient has been assessed as having a CMAI aggressive behavior score of greater than or equal to 15.

In some more particular embodiments, the patient has been assessed as having a CMAI aggressive behavior score of greater than or equal to 16.

In some more particular embodiments, the patient has been assessed as having a CMAI aggressive behavior score of greater than or equal to 17.

In some more particular embodiments, the patient has been assessed as having a CMAI aggressive behavior score of greater than or equal to 18.

In some more particular embodiments, the patient has been assessed as having a CMAI aggressive behavior score of greater than or equal to 19.

In some more particular embodiments, the patient has been assessed as having a CMAI aggressive behavior score of greater than or equal to 20.

In some more particular embodiments, the patient has been assessed as having a CMAI aggressive behavior score of greater than or equal to 21.

In some more particular embodiments, the patient has been assessed as having a CMAI aggressive behavior score of greater than or equal to 22.

In some more particular embodiments, the patient has been assessed as having a CMAI aggressive behavior score of greater than or equal to 23.

In some more particular embodiments, the patient has been assessed as having a CMAI aggressive behavior score of greater than or equal to 24.

In some more particular embodiments, the patient has been assessed as having a CMAI aggressive behavior score of greater than or equal to 25.

In some more particular embodiments, the patient has been assessed as having a CMAI aggressive behavior score of greater than or equal to 26.

In some more particular embodiments, the patient has been assessed as having a CMAI aggressive behavior score of greater than or equal to 27.

In some more particular embodiments, the patient has been assessed as having a CMAI aggressive behavior score of greater than or equal to 28.

In some more particular embodiments, the patient has been assessed as having a CMAI aggressive behavior score of greater than or equal to 29.

In some more particular embodiments, the patient has been assessed as having a CMAI aggressive behavior score of greater than or equal to 30.

In some more particular embodiments, the patient has been assessed as having a CMAI aggressive behavior score of greater than or equal to 31.

In some more particular embodiments, the patient has been assessed as having a CMAI aggressive behavior score of greater than or equal to 32.

In some more particular embodiments, the patient has been assessed as having a CMAI aggressive behavior score of greater than or equal to 33.

In some more particular embodiments, the patient has been assessed as having a CMAI aggressive behavior score of greater than or equal to 34.

In some more particular embodiments, the patient has been assessed as having a CMAI aggressive behavior score of greater than or equal to 35.

In some more particular embodiments, the patient has been assessed as having a CMAI aggressive behavior score of greater than or equal to 36.

In some more particular embodiments, the patient has been assessed as having a CMAI aggressive behavior score of greater than or equal to 37.

In some more particular embodiments, the patient has been assessed as having a CMAI aggressive behavior score of greater than or equal to 38.

In some more particular embodiments, the patient has been assessed as having a CMAI aggressive behavior score of greater than or equal to 39.

In some more particular embodiments, the patient has been assessed as having a CMAI aggressive behavior score of greater than or equal to 40.

In some more particular embodiments, the method comprises determining the score in the patient prior to the administering step for at least one of the following CMAI aggressive behavior items (i.e., “aggressive behaviors”) (assessed according to the method described elsewhere herein for assessing the CMAI total score according to the CMAI Manual, except that the score based on frequency of occurrence only needs to be determined for the at least one of the CMAI aggressive behavior items according to the methods of these embodiments):
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; or
12) cursing or verbal aggression.

The following aggressive behavior items:
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; and
12) cursing or verbal aggression
   are also referred to as “F1” behaviors.

In some more particular embodiments, the patient has been assessed to have a score of 2 or greater for at least one of CMAI aggressive behavior items 1) to 12) hereinabove.

In some more particular embodiments, the patient has been assessed to have a score of 2 to 7, such as 2 to 5, for at least one of CMAI aggressive behavior items 1) to 12) hereinabove.

In some embodiments of a method disclosed herein, the method comprises determining the CMAI total score in the patient following the administering step.

In some embodiments, the difference between the CMAI total score in the patient prior to the administering step and the CMAI total score in the patient following the administering step is from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40

In some embodiments, the CMAI total score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI total score in the patient prior to the administering step.

In some embodiments of a method disclosed herein, the method comprises determining the CMAI aggressive behavior score in the patient following the administering step.

In some embodiments, the difference between the CMAI aggressive behavior score in the patient prior to the administering step and the CMAI aggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI aggressive behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI aggressive behavior score in the patient prior to the administering step.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90 prior to the administering step, and the method comprises determining the CMAI total score in the patient following the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI aggressive behavior score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI aggressive behavior score in the patient prior to said administration, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40 prior to the administering step, and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI aggressive behavior score in the patient following the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI aggressive behavior score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI aggressive behavior score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI aggressive behavior score in the patient prior to the administering step and the CMAI aggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI aggressive behavior score in the patient prior to said administration, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40 prior to the administering step and the method comprises determining the CMAI aggressive behavior score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI aggressive behavior score in the patient prior to the administering step and the CMAI aggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments provided herein is a method of treating agitation associated with Alzheimer's disease in a patient that has been diagnosed as having Alzheimer's disease,

wherein the patient is 65 years old or older,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 70 years old or older,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is from 80 years old to 85 years old,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) total score in the patient is less than 50;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) total score in the patient is less than 65;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has been treated or is being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has been treated or is being treated with an SSRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has been treated or is being treated with an SNRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 17 to 24,
wherein the method comprises:
a) determining the Cohen-Mansfield agitation inventory (CMAI) total score in the patient;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is a male,
wherein the method comprises:
a) determining the Cohen-Mansfield agitation inventory (CMAI) total score in the patient;
and
b) administering to the patient effective amounts of d6-DM and quinidine sulfate,

In some embodiments provided herein is a method of treating agitation associated with Alzheimer's disease in a patient that has been diagnosed as having Alzheimer's disease,

wherein the patient is a male,
wherein the method comprises:
a) determining the Cohen-Mansfield agitation inventory (CMAI) total score in the patient;
and
b) administering to the patient effective amounts of d6-DM and quinidine sulfate, wherein following the administering step, the CMAI total score is less than the CMAI total score prior to the administering step in the patient, wherein the decrease in the CMAI total score is at least three points greater than a decrease in the CMAI total score in a female patient following administering of the same amounts of d6-DM and quinidine sulfate.

In some embodiments provided herein is a method of treating agitation associated with Alzheimer's disease in a patient that has been diagnosed as having Alzheimer's disease,

wherein the patient is greater or equal to 65 years old,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 75 years old or less,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 80 years old or older,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with an antipsychotic,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has not been treated and is not being treated with an antipsychotic following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) total score in the patient;
and
b) administering to the patient effective amounts of d6-DM and quinidine sulfate, wherein following the administration of the amounts, the CMAI total score is less than the CMAI total score prior to the administering step in the patient, wherein the decrease in the CMAI total score is greater, such as at least three points greater, than a decrease in the CMAI total score in a patient who has not been treated and is not being treated with memantime following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with a benzodiazepine,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) total score in the patient;
and
b) administering to the patient effective amounts of d6-DM and quinidine sulfate.
wherein following the administration of the amounts, the CMAI total score is less than the CMAI total score prior to the administering step in the patient, wherein the decrease in the CMAI total score is greater, such as at least three points greater, than a decrease in the CMAI total score in a patient who has not been treated and is not being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 13 to 19,
wherein the method comprises:
a) determining the Cohen-Mansfield agitation inventory (CMAI) total score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 65 years old or older,
wherein the method comprises:
a) determining the Cohen-Mansfield agitation inventory (CMAI) aggressive behavior score in the patient:
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is 70 years old or older,
wherein the method comprises:
a) determining the Cohen-Mansfield agitation inventory (CMAI) aggressive behavior score in the patient:
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient is from 80 years old to 85 years old,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is less than 50;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is less than 65;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with an SSRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with an SNRI following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 17 to 24,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has been treated or is being treated with an antipsychotic following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has not been treated and is not being treated with memantine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.
wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a patient who has not been treated and is not being treated with a benzodiazepine following administering of the same amounts of d6-DM and quinidine sulfate.

In some embodiments provided herein is a method of treating agitation associated with Alzheimer's disease in a patient that has been diagnosed as having Alzheimer's disease,

wherein the patient has an MMSE score from 13 to 19,
wherein the method comprises:
a) determining the Cohen-Mansfield agitation inventory (CMAI) aggressive behavior score in the patient;
and
b) administering to the patient effective amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.
   wherein following the administration of the amounts, the CMAI aggressive behavior score is less than the CMAI aggressive behavior score prior to the administering step in the patient by a first amount.

In some more particular embodiments the first amount is equal to at least three points in the CMAI aggressive behavior score.

In some more particular embodiments the first amount is greater, such as at least three points greater, than a decrease in the CMAI aggressive behavior score in a second patient following administering of the same amounts of d6-DM and quinidine sulfate to the second patient, wherein the method comprises determining that the Cohen-Mansfield agitation inventory (CMAI) aggressive behavior score in the second patient is less than 15 prior to the administering of the same amounts.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.
   wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a second patient following administering of the same amounts of d6-DM and quinidine sulfate to the second patient, wherein the method comprises determining that the Cohen-Mansfield agitation inventory (CMAI) aggressive behavior score in the second patient is less than 15 prior to the administering of the same amounts.

In some embodiments provided herein is a method of treating agitation associated with Alzheimer's disease in a patient that has been diagnosed as having Alzheimer's disease,

wherein the method comprises:
a) determining that the Cohen-Mansfield agitation inventory (CMAI) aggressive behavior score in the patient is greater than or equal to 15;
   and
b) administering to the patient effective amounts of d6-DM and quinidine sulfate.
   wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a second patient following administering of the same amounts of d6-DM and quinidine sulfate to the second patient, wherein the method comprises determining that the Cohen-Mansfield agitation inventory (CMAI) aggressive behavior score in the second patient is less than 15 prior to the administering of the same amounts.

In some more particular embodiments, the amount of d6-DM is 42.63 mg.

In some more particular embodiments, the amount of d6-DM is 28 mg.

In some more particular embodiments, the amount of d6-DM is 18 mg.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with an SSRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with an SNRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease, wherein the patient wherein the patient has been treated or is being treated with an antipsychotic,

wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has been treated or is being treated with an antipsychotic following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has not been treated and is not being treated with memantine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.
wherein following the administration of the amounts, the CMAI physically nonaggressive behavior score is less than the CMAI physically nonaggressive behavior score prior to the administering step in the patient by a second amount.

In some more particular embodiments the second amount is equal to at least three points in the CMAI physically nonaggressive behavior score.

In some more particular embodiments the second amount is greater, such as at least three points greater, than a decrease in the CMAI physically nonaggressive behavior score in a patient who has not been treated and is not being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SSRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with an SSRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has not been treated and is not being treated with an SNRI,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with an SNRI following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with an antipsychotic,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has been treated or is being treated with an antipsychotic following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient has been treated or is being treated with memantine,
wherein the method comprises:
a) determining the Cohen-Mansfield agitation inventory (CMAI) aggressive behavior score in the patient;
and
b) administering to the patient effective amounts of d6-DM and quinidine sulfate, wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has not been treated and is not being treated with memantine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some embodiments provided herein is a method of treating agitation associated with Alzheimer's disease in a patient that has been diagnosed as having Alzheimer's disease,

wherein the patient wherein the patient has been treated or is being treated with a benzodiazepine,
wherein the method comprises:
a) determining the Cohen-Mansfield agitation inventory (CMAI) aggressive behavior score in the patient;
and
b) administering to the patient effective amounts of d6-DM and quinidine sulfate.
wherein following the administration of the amounts, the CMAI verbally agitated behavior score is less than the CMAI verbally agitated behavior score prior to the administering step in the patient by a third amount.

In some more particular embodiments the third amount is equal to at least three points in the CMAI verbally agitated behavior score.

In some more particular embodiments the third amount is greater, such as at least three points greater, than a decrease in the CMAI verbally agitated behavior score in a patient who has not been treated and is not being treated with a benzodiazepine following administering of the same amounts of d6-DM and quinidine sulfate.

In some embodiments, the Cohen-Mansfield agitation inventory (CMAI) aggressive behavior score in the patient is determined in step a) to be greater than or equal to 15 prior to the administering step.

In some more particular embodiments, the amount of d6-DM is 42.63 mg.

In some more particular embodiments, the amount of d6-DM is 28 mg.

In some more particular embodiments, the amount of d6-DM is 18 mg.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI physically nonaggressive behavior score that is the same as the CMAI physically nonaggressive behavior score prior to the administering step or that differs from the CMAI physically nonaggressive behavior score prior to the administering step by no more than 7, 6, 5, 4, 3, 2, or 1.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI physically nonaggressive behavior score that is the same or at least 1 less, 2 less, 3 less, 4 less, 5 less, 6 less, or 7 less than the CMAI physically nonaggressive behavior score prior to the administering step.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI verbally agitated behavior score that is the same as the CMAI verbally agitated behavior score prior to the administering step or that differs from the CMAI verbally agitated behavior score prior to the administering step by no more than 7, 6, 5, 4, 3, 2, or 1.

In some more particular embodiments, the method comprises determining that the patient following the administering step has a CMAI aggressive behavior score that is at least 3 less, such as 3 to 6 less, than the CMAI aggressive behavior score prior to the administering step and optionally a CMAI verbally agitated behavior score that is the same or at least 1 less, 2 less, 3 less, 4 less, 5 less, 6 less, or 7 less than the CMAI verbally agitated behavior score prior to the administering step.

In some embodiments, the CMAI aggressive behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI aggressive behavior score in the patient prior to the administering step.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI physically nonaggressive behavior score in the patient prior to said administration.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMA1 total score in the patient prior to said administration.

In some embodiments, the present disclosure provides a method of treating physically nonaggressive agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI physically nonaggressive behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI physically nonaggressive behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30.

In some more particular embodiments, the patient has been assessed as having a CMAI physically nonaggressive behavior score of greater than or equal to 10.

In some more particular embodiments, the patient has been assessed as having a CMAI physically nonaggressive behavior score of greater than or equal to 11.

In some more particular embodiments, the patient has been assessed as having a CMAI physically nonaggressive behavior score of greater than or equal to 12.

In some more particular embodiments, the patient has been assessed as having a CMAI physically nonaggressive behavior score of greater than or equal to 13.

In some more particular embodiments, the patient has been assessed as having a CMAI physically nonaggressive behavior score of greater than or equal to 14.

In some more particular embodiments, the patient has been assessed as having a CMAI physically nonaggressive behavior score of greater than or equal to 15.

In some more particular embodiments, the patient has been assessed as having a CMAI physically nonaggressive behavior score of greater than or equal to 16.

In some more particular embodiments, the patient has been assessed as having a CMAI physically nonaggressive behavior score of greater than or equal to 17.

In some more particular embodiments, the patient has been assessed as having a CMAI physically nonaggressive behavior score of greater than or equal to 18.

In some more particular embodiments, the patient has been assessed as having a CMAI physically nonaggressive behavior score of greater than or equal to 19.

In some more particular embodiments, the patient has been assessed as having a CMAI physically nonaggressive behavior score of greater than or equal to 20.

In some more particular embodiments, the patient has been assessed as having a CMAI physically nonaggressive behavior score of greater than or equal to 21.

In some more particular embodiments, the patient has been assessed as having a CMAI physically nonaggressive behavior score of greater than or equal to 22.

In some more particular embodiments, the patient has been assessed as having a CMAI physically nonaggressive behavior score of greater than or equal to 23.

In some more particular embodiments, the patient has been assessed as having a CMAI physically nonaggressive behavior score of greater than or equal to 24.

In some more particular embodiments, the patient has been assessed as having a CMAI physically nonaggressive behavior score of greater than or equal to 25.

In some more particular embodiments, the patient has been assessed as having a CMAI physically nonaggressive behavior score of greater than or equal to 26.

In some more particular embodiments, the patient has been assessed as having a CMAI physically nonaggressive behavior score of greater than or equal to 27.

In some more particular embodiments, the patient has been assessed as having a CMAI physically nonaggressive behavior score of greater than or equal to 28.

In some more particular embodiments, the patient has been assessed as having a CMAI physically nonaggressive behavior score of greater than or equal to 29.

In some more particular embodiments, the patient has been assessed as having a CMAI physically nonaggressive behavior score of greater than or equal to 30.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has
   a. at least one physically nonaggressive behavior occurring once or twice a day;
   b. at least two physically nonaggressive behaviors occurring at least three times per week;
   c. at least three physically nonaggressive behaviors occurring, at least one time per week; and/or
   d. at least four physically nonaggressive behaviors occurring less than once per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI aggressive behavior score in the patient prior to the administering step;
2) administering to the patient therapeutically effective amounts of d6-DM and quinidine sulfate; and
3) determining the CMAI aggressive behavior score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI physically nonaggressive behavior score in the patient prior to the administering step;
2) administering to the patient therapeutically effective amounts of d6-DM and quinidine sulfate; and
3) determining the CMAI physically nonaggressive behavior score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI physically nonaggressive behavior score in the patient prior to the administering step;
2) determining that the patient prior to the administering step has
   a. at least one physically nonaggressive behavior occurring once or twice a day;
   b. at least two physically nonaggressive behaviors occurring at least three times per week;
   c. at least three physically nonaggressive behaviors occurring, at least one time per week; and/or
   d. at least four physically nonaggressive behaviors occurring less than once per week;
3) administering to the patient therapeutically effective amounts of d6-DM and quinidine sulfate; and
4) determining the CMAI physically nonaggressive behavior score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least one physically nonaggressive behavior occurring once or twice a day;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least two physically nonaggressive behaviors occurring at least three times per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least three physically nonaggressive behaviors occurring at least one time per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least four physically nonaggressive behaviors occurring less than once per week;
3) administering to the patient therapeutically effective amounts of d6-DM and quinidine sulfate; and
4) determining the CMAI total score in the patient following the administering step.

In some embodiments of a method disclosed herein, the method comprises determining the CMAI physically nonaggressive behavior score in the patient following the administering step.

In some embodiments, the difference between the CMAI physically nonaggressive behavior score in the patient prior to the administering step and the CMAI physically nonaggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI physically nonaggressive behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI physically nonaggressive behavior score in the patient prior to the administering step.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the CMAI verbally agitated behavior score in the patient prior to said administration.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating verbal agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI verbally agitated behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI verbally agitated behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25.

In some more particular embodiments, the patient has been assessed as having a CMAI verbally agitated behavior score of greater than or equal to 8.

In some more particular embodiments, the patient has been assessed as having a CMAI verbally agitated behavior score of greater than or equal to 9.

In some more particular embodiments, the patient has been assessed as having a CMAI verbally agitated behavior score of greater than or equal to 10.

In some more particular embodiments, the patient has been assessed as having a CMAI verbally agitated behavior score of greater than or equal to 11.

In some more particular embodiments, the patient has been assessed as having a CMAI verbally agitated behavior score of greater than or equal to 12.

In some more particular embodiments, the patient has been assessed as having a CMAI verbally agitated behavior score of greater than or equal to 13.

In some more particular embodiments, the patient has been assessed as having a CMAI verbally agitated behavior score of greater than or equal to 14.

In some more particular embodiments, the patient has been assessed as having a CMAI verbally agitated behavior score of greater than or equal to 15.

In some more particular embodiments, the patient has been assessed as having a CMAI verbally agitated behavior score of greater than or equal to 16.

In some more particular embodiments, the patient has been assessed as having a CMAI verbally agitated behavior score of greater than or equal to 17.

In some more particular embodiments, the patient has been assessed as having a CMAI verbally agitated behavior score of greater than or equal to 18.

In some more particular embodiments, the patient has been assessed as having a CMAI verbally agitated behavior score of greater than or equal to 19.

In some more particular embodiments, the patient has been assessed as having a CMAI verbally agitated behavior score of greater than or equal to 20.

In some more particular embodiments, the patient has been assessed as having a CMAI verbally agitated behavior score of greater than or equal to 21.

In some more particular embodiments, the patient has been assessed as having a CMAI verbally agitated behavior score of greater than or equal to 22.

In some more particular embodiments, the patient has been assessed as having a CMAI verbally agitated behavior score of greater than or equal to 23.

In some more particular embodiments, the patient has been assessed as having a CMAI verbally agitated behavior score of greater than or equal to 24.

In some more particular embodiments, the patient has been assessed as having a CMAI verbally agitated behavior score of greater than or equal to 25.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has
   a. at least one verbally agitated behavior occurring once or twice a day;
   b. at least two verbally agitated behaviors occurring at least three times per week;
   c. at least three verbally agitated behaviors occurring, at least one time per week; and/or
   d. at least four verbally agitated behaviors occurring less than once per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least one verbally agitated behavior occurring once or twice a day;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least two verbally agitated behaviors occurring at least three times per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least three verbally agitated behaviors occurring at least one time per week;
3) administering to the patient therapeutically effective amounts of d6-DM and quinidine sulfate; and
4) determining the CMAI total score in the patient following the administering step.

In some embodiments of a method disclosed herein, the present disclosure provides a method of treating agitation associated with Alzheimer's disease in a patient that has been diagnosed as having Alzheimer's disease, comprising
1) determining the CMAI total score in the patient prior to the administering step;
2) determining that the patient prior to the administering step has at least four verbally agitated behaviors occurring less than once per week;
3) administering to the patient therapeutically effective amounts of d6-DM and quinidine sulfate; and
4) determining the CMAI total score in the patient following the administering step.

In some embodiments of a method disclosed herein, the method comprises determining the CMAI verbally agitated behavior score in the patient following the administering step.

In some embodiments, the difference between the CMAI verbally agitated behavior score in the patient prior to the administering step and the CMAI verbally agitated behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI verbally agitated behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI verbally agitated behavior score in the patient prior to the administering step.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining in the patient prior to the administering step the CMAI total score and the score for at least one of the following CMAI aggressive behaviors:
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; or
12) cursing or verbal aggression.
   wherein the method comprises determining the score in the patient following the administering step for at least one of CMAI aggressive behavior items 1) to 12).

In some more particular embodiments, the patient has been assessed to have a score of 2 or greater for at least one of CMAI aggressive behavior items 1) to 12) prior to the administering step.

In some more particular embodiments, the difference between the score in the patient prior to the administering step for at least one of CMAI aggressive behavior items 1) to 12) and the score in the patient following the administering step for the at least one of CMAI aggressive behavior items 1) to 12) is at least 1.

In some more particular embodiments, the difference between the score in the patient prior to the administering step for at least one of CMAI aggressive behavior items 1) to 12) hereinabove and the score in the patient following the administering step for the at least one of CMAI aggressive behavior items 1) to 12) is at least 1, such as 1 to 6, such as 1, 2, 3, 4, 5, or 6.

In some more particular embodiments, the method comprises determining the score in the patient prior to the administering step for at least one of the following CMAI physically nonaggressive behavior items (“physically nonaggressive behaviors”) (assessed according to the method described elsewhere herein for assessing the CMAI total score according to the CMAI Manual, except that the score based on frequency of occurrence only needs to be determined for the at least one of the CMAI physically nonaggressive behavior items according to the methods of these embodiments):
1) pacing and/or aimless wandering
2) trying to get to a different place;
3) general restlessness;
4) inappropriate dressing or disrobing;
5) handling things inappropriately; or
6) performing repetitious mannerisms.

The physically nonaggressive behavior items above are also referred to as “F2” behaviors.

In some more particular embodiments, the patient has been assessed to have a score of 2 or greater for at least one of CMAI physically nonaggressive behavior items 1) to 6) hereinabove.

In some more particular embodiments, the patient has been assessed to have a score of 2 to 7, such as 2 to 5, for at least one of CMAI physically nonaggressive behavior items 1) to 6) hereinabove.

In some embodiments of a method disclosed herein, the method comprises determining the CMAI total score in the patient following the administering step.

In some embodiments, the difference between the CMAI total score in the patient prior to the administering step and the CMAI total score in the patient following the administering step is from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40

In some embodiments, the CMAI total score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI total score in the patient prior to the administering step.

In some embodiments of a method disclosed herein, the method comprises determining the CMAI physically nonaggressive behavior score in the patient following the administering step.

In some embodiments, the difference between the CMAI physically nonaggressive behavior score in the patient prior to the administering step and the CMAI physically nonaggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI physically nonaggressive behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI physically nonaggressive behavior score in the patient prior to the administering step.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90 prior to the administering step, and the method comprises determining the CMAI total score in the patient following the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI physically nonaggressive behavior score in the patient prior to said administration, wherein the patient has been assessed as having a Cohen-Mansfield agitation inventory (CMAI) total score of greater than or equal to 33, such as 33 to 150, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI physically nonaggressive behavior score in the patient prior to said administration, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30 prior to the administering step, and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI physically nonaggressive behavior score in the patient following the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI physically nonaggressive behavior score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI physically nonaggressive behavior score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI physically nonaggressive behavior score in the patient prior to the administering step and the CMAI physically nonaggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI physically nonaggressive behavior score in the patient prior to said administration, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30 prior to the administering step and the method comprises determining the CMAI physically nonaggressive behavior score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI physically nonaggressive behavior score in the patient prior to the administering step and the CMAI physically nonaggressive behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining in the patient prior to the administering step the CMAI total score and the score for at least one of the following CMAI physically nonaggressive behaviors:
1) pacing and/or aimless wandering
2) trying to get to a different place;
3) general restlessness;
4) inappropriate dressing or disrobing;
5) handling things inappropriately;
or
6) performing repetitious mannerisms.
   wherein the method comprises determining the score in the patient following the administering step for at least one of CMAI physically nonaggressive behavior items 1) to 6).

In some more particular embodiments, the patient has been assessed to have a score of 2 or greater for at least one of CMAI physically nonaggressive behavior items 1) to 6) prior to the administering step.

In some more particular embodiments, the difference between the score in the patient prior to the administering step for at least one of CMAI physically nonaggressive behavior items 1) to 6) and the score in the patient following the administering step for the at least one of CMAI physically nonaggressive behavior items 1) to 6) is at least 1.

In some more particular embodiments, the method comprises determining the score in the patient following the administering step for at least one of the following CMAI physically nonaggressive behavior items:
1) pacing and/or aimless wandering
2) trying to get to a different place;
3) general restlessness;
4) inappropriate dressing or disrobing;
5) handling things inappropriately;
or
6) performing repetitious mannerisms.

In some more particular embodiments, the difference between the score in the patient prior to the administering step for at least one of CMAI physically nonaggressive behavior items 1) to 6) hereinabove and the score in the patient following the administering step for the at least one of CMAI physically nonaggressive behavior items 1) to 6) is at least 1, such as 1 to 6, such as 1, 2, 3, 4, 5, or 6.

In some more particular embodiments, the method comprises determining the score in the patient prior to the administering step for at least one of the following CMAI verbally agitated behavior items (“verbally agitated behaviors”) (assessed according to the method described elsewhere herein for assessing the CMAI total score according to the CMAI Manual, except that the score based on frequency of occurrence only needs to be determined for the at least one of the CMAI verbally agitated behavior items according to the methods of these embodiments):
1) complaining;
2) constant unwarranted requests for attention and/or help;
3) repetitive sentences or questions; or
4) negativism.

The verbally agitated behavior items above are also referred to as “F3” behaviors.

In some more particular embodiments, the patient has been assessed to have a score of 2 or greater for at least one of CMAI verbally agitated behavior items 1) to 4) hereinabove.

In some more particular embodiments, the patient has been assessed to have a score of 2 to 7, such as 2 to 5, for at least one of CMAI verbally agitated behavior items 1) to 4) hereinabove.

In some embodiments of a method disclosed herein, the method comprises determining the CMAI total score in the patient following the administering step.

In some embodiments, the difference between the CMAI total score in the patient prior to the administering step and the CMAI total score in the patient following the administering step is from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the CMAI total score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% c lower, such as 15% lower, than the CMAI total score in the patient prior to the administering step.

In some embodiments of a method disclosed herein, the method comprises determining the CMAI verbally agitated behavior score in the patient following the administering step.

In some embodiments, the difference between the CMAI verbally agitated behavior score in the patient prior to the administering step and the CMAI verbally agitated behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the CMAI verbally agitated behavior score in the patient following the administering step is at least 1% lower, such as 1% to 70% lower, such as 2% to 65% lower, such as 3% to 60% lower, such as 4% to 55% lower, such as 5% to 50% lower, such as 6% to 45% lower, such as 7% to 40% lower, such as 8% to 35% lower, such as 9% to 30% lower, such as 10% to 25% lower, such as 10% to 20% lower, such as 15% lower, than the CMAI verbally agitated behavior score in the patient prior to the administering step.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90 prior to the administering step, and the method comprises determining the CMAI total score in the patient following the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI verbally agitated behavior score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI verbally agitated behavior score in the patient prior to said administration, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25 prior to the administering step, and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI verbally agitated behavior score in the patient following the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI verbally agitated behavior score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI verbally agitated behavior score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI verbally agitated behavior score in the patient prior to the administering step and the CMAI verbally agitated behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the CMAI verbally agitated behavior score in the patient prior to said administration, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25 prior to the administering step and the method comprises determining the CMAI verbally agitated behavior score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate. In some more particular embodiments, the difference between the CMAI verbally agitated behavior score in the patient prior to the administering step and the CMAI verbally agitated behavior score in the patient following the administering step is at least 1, such as 1 to 20, such as 2 to 20, such as 3 to 15, such as 4 to 15, such as 5 to 15, such as 1, 2, 3, 4, 5, 6, 7, 8, 9, 10, 11, 12, 13, 14 or 15.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining in the patient prior to the administering step the CMAI total score and the score for at least one of the following CMAI verbally agitated behaviors:
1) complaining;
2) constant unwarranted requests for attention and/or help;
3) repetitive sentences or questions;
or
4) negativism.
   wherein the method comprises determining the score in the patient following the administering step for at least one of CMAI verbally agitated behavior items 1) to 4).

In some more particular embodiments, the patient has been assessed to have a score of 2 or greater for at least one of CMAI verbally agitated behavior items 1) to 4) prior to the administering step.

In some more particular embodiments, the difference between the score in the patient prior to the administering step for at least one of CMAI verbally agitated behavior items 1) to 4) and the score in the patient following the administering step for the at least one of CMAI verbally agitated behavior items 1) to 4) is at least 1.

In some more particular embodiments, the method comprises determining the score in the patient following the administering step for at least one of the following CMAI verbally agitated behavior items:
1) complaining,
2) constant unwarranted requests for attention and/or help;
3) repetitive sentences or questions; or
4) negativism.

In some more particular embodiments, the difference between the score in the patient prior to the administering step for at least one of CMAI verbally agitated behavior items 1) to 6) hereinabove and the score in the patient following the administering step for the at least one of CMAI verbally agitated behavior items 1) to 6) is at least 1, such as 1 to 6, such as 1, 2, 3, 4, 5, or 6.

In some embodiments the deuterated [d6]-dextromethorphan hydrobromide (d6-DM) is a deuterated [d6]-dextromethorphan hydrobromide hydrate, such as [d6]-dextromethorphan hydrobromide monohydrate.

In some embodiments of a method disclosed herein, the method comprises determining the NPI-AA score in the patient prior to the administering step.

In some embodiments the NPI-AA score prior to the administering step is equal to or greater than 4.

In some embodiments, the NPI-AA score prior to the administering step is equal to 3, such as 3 to 12, such as 3, 4, 5, 6, 7, 8, 9, or 10.

In some embodiments of a method disclosed herein, the method comprises determining the NPI Aberrant Motor Behavior domain score in the patient prior to the administering step.

In some embodiments the NPI Aberrant Motor Behavior domain score prior to the administering step is greater than or equal to 2, such as 2 to 12, such as 2, 3, 4, 5, 6, 7, 8, 9, or 10.

In some embodiments of a method disclosed herein, the method comprises determining the NPI Irritability/Lability domain score in the patient prior to the administering step.

In some embodiments the NPI Irritability/Lability domain score prior to the administering step is equal to or greater than 2, such as 2 to 12, such as 2, 3, 4, 5, 6, 7, 8, 9, or 10.

In some embodiments of a method disclosed herein, the method comprises determining the NPI total score in the patient prior to the administering step.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the NPI-AA score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI-AA score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI-AA score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI-AA score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10. In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the NPI Aberrant Motor Behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI Aberrant Motor Behavior score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI Aberrant Motor Behavior score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI Aberrant Motor Behavior score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Aberrant Motor Behavior score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Aberrant Motor Behavior domain score greater than or equal to 2, such as 2 to 12, such as 2, 3, 4, 5, 6, 7, 8, 9, or 10.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the NPI Irritability/Lability domain score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI Irritability/Lability domain score in the patient prior to said administration.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI Irritability/Lability domain score in the patient prior to said administration.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI Irritability/Lability domain score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Irritability/Lability domain score of greater than or equal to 4.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI Irritability/Lability domain score greater than or equal to 2, such as 2 to 12, such as 2, 3, 4, 5, 6, 7, 8, 9, or 10.

In some embodiments of a method disclosed herein, the method comprises determining the NPI-AA score in the patient following the administering step.

In some embodiments, the difference between the NPI-AA score in the patient prior to the administering step and the NPI-AA score in the patient following the administering step is at least 1, such as 1 to 9, such as 1, 2, 3, 4, 5, 6, or 7.

In some embodiments of a method disclosed herein, the method comprises determining the NPI Aberrant Motor Behavior domain score in the patient following the administering step.

In some embodiments, the difference between the NPI Aberrant Motor Behavior domain score in the patient prior to the administering step and the NPI Aberrant Motor Behavior domain score in the patient following the administering step is at least 1, such as 1 to 9, such as 1, 2, 3, 4, 5, 6, or 7.

In some embodiments of a method disclosed herein, the method comprises determining the NPI Irritability/Lability domain score in the patient following the administering step.

In some embodiments, the difference between the NPI Irritability/Lability domain score in the patient prior to the administering step and the NPI Irritability/Lability domain score in the patient following the administering step is at least 1, such as 1 to 9, such as 1, 2, 3, 4, 5, 6, or 7.

In some embodiments of a method disclosed herein, the method comprises determining the NPI total score in the patient following the administering step.

In some embodiments, the difference between the NPI total score in the patient prior to the administering step and the NPI total score in the patient following the administering step is at least 1, such as 1 to 9, such as 1, 2, 3, 4, 5, 6, or 7.

In some embodiments, provided herein is a method of treating agitation associated with Alzheimer's disease in a patient that has been diagnosed as having Alzheimer's disease, comprising:

(1) determining the CMAI total score in the patient prior to step (2);
(2) administering to the patient therapeutically effective amounts of d6-DM and quinidine sulfate; and
(3) determining that the difference between the CMAI total score in the patient prior to the administering step and the CMAI total score in the patient following the administering step is from 11 to 16.

In some embodiments, the CMAI total score in the patient following the administering step is from 12 to 15 less, such as from 13 to 15 less, such as 14.3 less, than the CMAI total score in the patient prior to the administering step.

In some embodiments, the subject prior to the administering step has one or more of:
i) a CMAI total score equal to the mean value±the standard deviation (SD) shown in Table 16 for AVP 786-18;
ii) a CMAI aggressive behavior score equal to the mean value±the standard deviation (SD) shown in Table 16 for AVP 786-18;
iii) a CMAI physically nonaggressive behavior score equal to the mean value±the standard deviation (SD) shown in Table 16 for AVP 786-18;
   or
iv) a CMAI verbal agitation score equal to the mean values±the standard deviation (SD) shown in Table 16 for AVP 786-18

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value±the standard deviation (SD) shown in Table 16 for AVP 786-18.

In some embodiments, the subject prior to the administering step has one or more of:
i) a CMAI total score equal to the mean value±the standard deviation (SD) shown in Table 16 for AVP 786-18;
   or
ii) a CMAI aggressive behavior score equal to the mean value±the standard deviation (SD) shown in Table 16 for AVP 786-18.

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value±the standard deviation (SD) shown in Table 16 for AVP 786-18.

In some embodiments, the subject prior to the administering step has one or more of:
i) a CMAI total score equal to the mean value±the standard deviation (SD) shown in Table 16 for AVP 786-18;
   or
ii) a CMAI physically nonaggressive behavior score equal to the mean value±the standard deviation (SD) shown in Table 16 for AVP 786-18.

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value±the standard deviation (SD) shown in Table 16 for AVP 786-18.

In some embodiments, the subject prior to the administering step has one or more of:
i) a CMAI total score equal to the mean value±the standard deviation (SD) shown in Table 16 for AVP 786-18;
   or
ii) a CMAI verbal agitation score equal to the mean values±the standard deviation (SD) shown in Table 16 for AVP 786-18.

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value±the standard deviation (SD) shown in Table 16 for AVP 786-18.

In some embodiments, the subject prior to the administering step has one or more of:
i) a CMAI aggressive behavior score equal to the mean value±the standard deviation (SD) shown in Table 16 for AVP 786-18;
ii) a CMAI physically nonaggressive behavior score equal to the mean value±the standard deviation (SD) shown in Table 16 for AVP 786-18; or
iii) a CMAI verbal agitation score equal to the mean values t the standard deviation (SD) shown in Table 16 for AVP 786-18

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value±the standard deviation (SD) shown in Table 16 for AVP 786-18.

In some embodiments, the subject prior to the administering step has one or more of:
i) a CMAI aggressive behavior score equal to the mean value±the standard deviation (SD) shown in Table 16 for AVP 786-18; or
ii) a CMAI physically nonaggressive behavior score equal to the mean value±the standard deviation (SD) shown in Table 16 for AVP 786-18;

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value±the standard deviation (SD) shown in Table 16 for AVP 786-18.

In some embodiments, the subject prior to the administering step has one or more of:
i) a CMAI aggressive behavior score equal to the mean value±the standard deviation (SD) shown in Table 16 for AVP 786-18;
   or
ii) a CMAI verbal agitation score equal to the mean values t the standard deviation (SD) shown in Table 16 for AVP 786-18.

In some more particular embodiments, the subject prior to the administering step also has one or more of:
i) a NPI total score equal to the mean value±the standard deviation (SD) shown in Table 16 for AVP 786-18;
ii) a NPI-Agitation/Aggression Domain score equal to the mean value±the standard deviation (SD) shown in Table 16 for AVP 786-18; or
iii) a CGIS-Agitation score equal to the mean value t the standard deviation (SD) shown in Table 16 for AVP 786-18.

In some embodiments, the subject prior to the administering step has one or more of:
i) a CMAI physically nonaggressive behavior score equal to the mean value±the standard deviation (SD) shown in Table 16 for AVP 786-18;
   or
ii) a CMAI verbal agitation score equal to the mean values±the standard deviation (SD) shown in Table 16 for AVP 786-18.

In some more particular embodiments, the subject prior to the administering step also has one or more of:
i) a NPI total score equal to the mean value±the standard deviation (SD) shown in Table 16 for AVP 786-18:
ii) a NPI-Agitation/Aggression Domain score equal to the mean value±the standard deviation (SD) shown in Table 16 for AVP 786-18; or
iii) a CGIS-Agitation score equal to the mean value t the standard deviation (SD) shown in Table 16 for AVP 786-18.

In some embodiments, the method comprises determining that the patient has a CMAI total score of 49 to 96, such as 72, prior to the administering step.

In some aspects of the foregoing embodiments, the CMAI total score following the administering step is determined 1 week after the administering step.

In some aspects of the foregoing embodiments, the CMAI total score following the administering step is determined 2 weeks after the administering step.

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI total score determined 1 week after the administering step is 5 to 8 less, such as 6 to 7 less, such as 6.5 less than the CMAI total score prior to the administering step.

In some aspects of the foregoing embodiments, the CMAI total score determined 2 weeks after the administering step is 7 to 11 less, such as 8 to 10 less, such as 9 less, such as 9.1 less than the CMAI total score prior to the administering step.

In some aspects of the foregoing embodiments, the CMAI total score determined 3 weeks after the administering step is 10 to 14 less, such as 11 to 13 less, such as 12 less, such as 11.9 less than the CMAI total score prior to the administering step.

In some aspects of the foregoing embodiments, the CMAI total score determined 6 weeks after the administering step is 11 to 15 less, such as 13 to 15 less, such as 14 less, such as 14.3 less than the CMAI total score prior to the administering step.

In some aspects of the foregoing embodiments, the CMAI total score determined 9 weeks after the administering step is 12 to 16 less, such as 13 to 16 less, such as 14 to 15 less, such as 14.8 less than the CMAI total score prior to the administering step.

In some aspects of the foregoing embodiments, the CMAI total score determined 12 weeks after the administering step is 13 to 17 less, such as 14 to 17 less, such as 15 to 16 less, such as 15.6 less than the CMAI total score prior to the administering step.

A method of treating agitation associated with Alzheimer's disease in a patient that has been diagnosed as having Alzheimer's disease, comprising.
(1) determining the CMAI total score in the patient prior to step (2);
(2) administering to the patient therapeutically effective amounts of d6-DM and quinidine sulfate; and
(3) determining that the difference between the CMAI total score in the patient prior to the administering step and the CMAI total score in the patient following the administering step is from 13 to 20,

In some embodiments, the CMAI total score in the patient following the administering step is 14 to 20 less, such as from 16 to 20 less, such as 18.9 less, than the CMAI total score in the patient prior to the administering step.

In some embodiments, the subject prior to the administering step has one or more of:
i) a CMAI total score equal to the mean value±the standard deviation (SD) shown in Table 64 for AVP 786-42.63;
ii) a CMAI aggressive behavior score equal to the mean value±the standard deviation (SD) shown in Table 64 for AVP 786-42.63,
iii) a CMAI physically nonaggressive behavior score equal to the mean value±the standard deviation (SD) shown in Table 64 for AVP 786-42.63;
or
iv) a CMAI verbal agitation score equal to the mean values±the standard deviation (SD) shown in Table 64 for AVP 786-42.63

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value t the standard deviation (SD) shown in Table 64 for AVP 786-42.63; or
iii) a CGIS-Agitation score equal to the mean value±the standard deviation (SD) shown in Table 64 for AVP 786-42.63.

In some embodiments, the subject prior to the administering step has one or more of:
i) a CMAI total score equal to the mean value±the standard deviation (SD) shown in Table 64 for AVP 786-42.63; or
ii) a CMAI aggressive behavior score equal to the mean value t the standard deviation (SD) shown in Table 64 for AVP 786-42.63.

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value±the standard deviation (SD) shown in Table 64 for AVP 786-42.63; or
iii) a CGIS-Agitation score equal to the mean value±the standard deviation (SD) shown in Table 64 for AVP 786-42.63.

In some embodiments, the subject prior to the administering step has one or more of:
i) a CMAI total score equal to the mean value±the standard deviation (SD) shown in Table 64 for AVP 786-42.63; or
ii) a CMAI physically nonaggressive behavior score equal to the mean value±the standard deviation (SD) shown in Table 64 for AVP 786-42.63.

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value±the standard deviation (SD) shown in Table 64 for AVP 786-42.63; or
iii) a CGIS-Agitation score equal to the mean value±the standard deviation (SD) shown in Table 64 for AVP 786-42.63.

In some embodiments, the subject prior to the administering step has one or more of:
i) a CMAI total score equal to the mean value±the standard deviation (SD) shown in Table 64 for AVP 786-42.63; or
ii) a CMAI verbal agitation score equal to the mean values±the standard deviation (SD) shown in Table 64 for AVP 786-42.63.

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value±the standard deviation (SD) shown in Table 64 for AVP 786-42.63; or
iii) a CGIS-Agitation score equal to the mean value±the standard deviation (SD) shown in Table 64 for AVP 786-42.63.

In some embodiments, the subject prior to the administering step has one or more of:
i) a CMAI aggressive behavior score equal to the mean value±the standard deviation (SD) shown in Table 64 for AVP 786-42.63,
ii) a CMAI physically nonaggressive behavior score equal to the mean value±the standard deviation (SD) shown in Table 64 for AVP 786-42.63;
or
iii) a CMAI verbal agitation score equal to the mean values±the standard deviation (SD) shown in Table 64 for AVP 786-42.63.

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value t the standard deviation (SD) shown in Table 64 for AVP 786-42.63, or
iii) a CGIS-Agitation score equal to the mean value±the standard deviation (SD) shown in Table 64 for AVP 786-42.63.

In some embodiments, the subject prior to the administering step has one or more of:
i) a CMAI aggressive behavior score equal to the mean value±the standard deviation (SD) shown in Table 64 for AVP 786-42.63; or
ii) a CMAI physically nonaggressive behavior score equal to the mean value±the standard deviation (SD) shown in Table 64 for AVP 786-42.63;

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value±the standard deviation (SD) shown in Table 64 for AVP 786-42.63, or
iii) a CGIS-Agitation score equal to the mean value±the standard deviation (SD) shown in Table 64 for AVP 786-42.63.

In some embodiments, the subject prior to the administering step has one or more of:
i) a CMAI aggressive behavior score equal to the mean value±the standard deviation (SD) shown in Table 64 for AVP 786-42.63; or
ii) a CMAI verbal agitation score equal to the mean values±the standard deviation (SD) shown in Table 64 for AVP 786-42.63.

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value t the standard deviation (SD) shown in Table 64 for AVP 786-42.63; or
iii) a CGIS-Agitation score equal to the mean value±the standard deviation (SD) shown in Table 64 for AVP 786-42.63.

In some embodiments, the subject prior to the administering step has one or more of:
i) a CMAI physically nonaggressive behavior score equal to the mean value±the standard deviation (SD) shown in Table 64 for AVP 786-42.63; or
ii) a CMAI verbal agitation score equal to the mean values±the standard deviation (SD) shown in Table 64 for AVP 786-42.63.

In some more particular embodiments, the subject prior to the administering step also has one or more of:
i) a NPI total score equal to the mean value±the standard deviation (SD) shown in Table 64 for AVP 786-42.63;
ii) a NPI-Agitation/Aggression Domain score equal to the mean value t the standard deviation (SD) shown in Table 64 for AVP 786-42.63; or
iii) a CGIS-Agitation score equal to the mean value±the standard deviation (SD) shown in Table 64 for AVP 786-42.63.

In some embodiments, the method comprises determining that the patient has a CMAI total score of 50 to 92, such as 71, prior to the administering step.

In some aspects of the foregoing embodiments, the CMAI total score following the administering step is determined 1 week after the administering step.

In some aspects of the foregoing embodiments, the CMAI total score following the administering step is determined 2 weeks after the administering step.

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI total score determined 1 week after the administering step is 7 to 9 less, such as 8 less, such as 8.1 less than the CMAI total score prior to the administering step.

In some aspects of the foregoing embodiments, the CMAI total score determined 2 weeks after the administering step is 9 to 13 less, such as 10 to 12 less, such as 11 less, such as 11.0 less than the CMAI total score prior to the administering step.

In some aspects of the foregoing embodiments, the CMAI total score determined 3 weeks after the administering step is 10 to 15 less, such as 11 to 14 less, such as 12 to 13 less, such as 12.6 less than the CMAI total score prior to the administering step.

In some aspects of the foregoing embodiments, the CMAI total score determined 6 weeks after the administering step is 12 to 16 less, such as 13 to 15 less, such as 14 less, such as 14.1 less than the CMAI total score prior to the administering step.

In some aspects of the foregoing embodiments, the CMAI total score determined 9 weeks after the administering step is 12 to 20 less, such as 14 to 19 less, such as 15 to 18 less, such as 17 less, such as 17.3 less than the CMAI total score prior to the administering step.

In some aspects of the foregoing embodiments, the CMAI total score determined 12 weeks after the administering step is 13 to 22 less, such as 15 to 21 less, such as 16 to 20 less, such as 17 to 19 less, such as 18.9 less than the CMAI total score prior to the administering step.

In some embodiments, provided herein is a method of treating agitation associated with Alzheimer's disease in a patient that has been diagnosed as having Alzheimer's disease, comprising:
(1) determining the CMAI aggressive behavior score in the patient prior to step (2);
(2) administering to the patient therapeutically effective amounts of d6-DM and quinidine sulfate; and
(3) determining that the difference between the CMAI aggressive behavior score in the patient prior to the administering step and the CMAI aggressive behavior score in the patient following the administering step is from 3 to 6, such as from 4 to 5.

In some embodiments, the CMAI aggressive behavior score in the patient following the administering step is from 3 to 6 less, such as from 4 to 5 less, such as from 4.4 to 4.8 less, than the CMAI aggressive behavior score in the patient prior to the administering step.

In some embodiments, the subject prior to the administering step has one or more of:
i) a CMAI total score equal to the mean value t the standard deviation (SD) shown in Table 16 for AVP 786-18;
ii) a CMAI aggressive behavior score equal to the mean value t the standard deviation (SD) shown in Table 16 for AVP 786-18;
iii) a CMAI physically nonaggressive behavior score equal to the mean value±the standard deviation (SD) shown in Table 16 for AVP 786-18; or
iv) a CMAI verbal agitation score equal to the mean values±the standard deviation (SD) shown in Table 16 for AVP 786-18.

In some more particular embodiments, the method comprises determining that the patient has a CMAI aggressive behavior score of 12 to 31, such as at least 15, prior to the administering step.

In some more particular embodiments, the method comprises determining that the patient has a CMAI total score of 49 to 96, such as 72, prior to the administering step.

In some more particular embodiments, the method comprises determining that the patient has a CMAI aggressive behavior score of 12 to 31, such as at least 15, prior to the administering step and a reduction in CMAI total score of from 11 to 16 following the administering step.

In some more particular embodiments, the method comprises determining that the patient following the administering step has a CMAI aggressive behavior score that differs from the CMAI aggressive behavior score prior to the administering step by 3 to 6 and optionally a CMAI physically nonaggressive behavior score that differs from the CMAI physically nonaggressive behavior score prior to the administering step by no more than 7, 6, 5, 4, 3, 2, or 1.

In some more particular embodiments, the method comprises determining that the patient following the administering step has a CMAI aggressive behavior score that is 3 to 6 less than the CMAI aggressive behavior score prior to the administering step and optionally a CMAI physically nonaggressive behavior score that is at least 1 less, 2 less, 3 less, 4 less, 5 less, 6 less, or 7 less than the CMAI physically nonaggressive behavior score prior to the administering step.

In some aspects of the foregoing embodiments, the CMAI total score following the administering step is determined 1 week after the administering step.

In some aspects of the foregoing embodiments, the CMAI total score following the administering step is determined 2 weeks after the administering step.

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI aggressive behavior score following the administering step is determined 1 week after the administering step.

In some aspects of the foregoing embodiments, the CMAI aggressive behavior score following the administering step is determined 2 weeks after the administering step.

In some aspects of the foregoing embodiments, the CMAI aggressive behavior score following the administering step is determined 3 weeks after the administering step.

In some aspects of the foregoing embodiments, the CMAI aggressive behavior score following the administering step is determined 6 weeks after the administering step.

In some aspects of the foregoing embodiments, the CMAI aggressive behavior score following the administering step is determined 9 weeks after the administering step.

In some aspects of the foregoing embodiments, the CMAI aggressive behavior score following the administering step is determined 12 weeks after the administering step.

In some aspects of the foregoing embodiments, the CMAI physically nonaggressive behavior score following the administering step is determined 1 week after the administering step.

In some aspects of the foregoing embodiments, the CMAI physically nonaggressive behavior score following the administering step is determined 2 weeks after the administering step.

In some aspects of the foregoing embodiments, the CMAI physically nonaggressive behavior score following the administering step is determined 3 weeks after the administering step.

In some aspects of the foregoing embodiments, the CMAI physically nonaggressive behavior score following the administering step is determined 6 weeks after the administering step.

In some aspects of the foregoing embodiments, the CMAI physically nonaggressive behavior score following the administering step is determined 9 weeks after the administering step.

In some aspects of the foregoing embodiments, the CMAI physically nonaggressive behavior score following the administering step is determined 12 weeks after the administering step.

In some aspects of the foregoing embodiments, the CMAI aggressive behavior score determined 1 week after the administering step is 1 to 3 less, such as 2 less, such as 1.59 less than the CMAI aggressive behavior score prior to the administering step.

In some aspects of the foregoing embodiments, the CMAI aggressive behavior score determined 2 weeks after the administering step is 2 to 4 less, such as 3 less, such as 2.8 less than the CMAI aggressive behavior score prior to the administering step.

In some aspects of the foregoing embodiments, the CMAI aggressive behavior score determined 3 weeks after the administering step is 3 to 5 less, such as 4 less, such as 3.8 less than the CMAI aggressive behavior score prior to the administering step.

In some aspects of the foregoing embodiments, the CMAI aggressive behavior score determined 6 weeks after the administering step is 3 to 6 less, such as 4 to 5 less, such as 4 less, such as 4.4 less than the CMAI aggressive behavior score prior to the administering step.

In some aspects of the foregoing embodiments, the CMAI aggressive behavior score determined 9 weeks after the administering step is 3 to 6 less, such as 4 to 5 less, such as 5 less, such as 4.7 less than the CMAI aggressive behavior score prior to the administering step.

In some aspects of the foregoing embodiments, the CMAI aggressive behavior score determined 12 weeks after the administering step is 3 to 6 less, such as 4 to 5 less, such as 5 less, such as 4.8 less than the CMAI aggressive behavior score prior to the administering step.

In some embodiments, provided herein is a method of treating agitation associated with Alzheimer's disease in a patient that has been diagnosed as having Alzheimer's disease, comprising:
(1) determining the CMAI aggressive behavior score in the patient prior to step (2);
(2) administering to the patient therapeutically effective amounts of d6-DM and quinidine sulfate; and
(3) determining that the difference between the CMAI aggressive behavior score in the patient prior to the administering step and the CMAI aggressive behavior score in the patient following the administering step is from 4 to 6.

In some embodiments, the CMAI aggressive behavior score in the patient following the administering step is from 4 to 5 less, or from 5 to 6 less, such as 5.1 less, than the CMAI aggressive behavior score in the patient prior to the administering step.

In some more particular embodiments, the method comprises determining that the patient has a CMAI aggressive behavior score of 11 to 29, such as at least 15, prior to the administering step.

In some more particular embodiments, the method comprises determining that the patient has a CMAI total score of 50 to 92, such as 71, prior to the administering step.

In some more particular embodiments, the method comprises determining that the patient has a CMAI aggressive behavior score of 11 to 29, such as at least 15, prior to the administering step and a reduction in CMAI total score from 13 to 22 following the administering step.

In some more particular embodiments, the method comprises determining that the patient following the administering step has a CMAI aggressive behavior score that differs from the CMAI aggressive behavior score prior to the administering step by 4 to 6 and optionally a CMAI physically nonaggressive behavior score that differs from the CMAI physically nonaggressive behavior score prior to the administering step by no more than 7, 6, 5, 4, 3, 2, or 1.

In some more particular embodiments, the method comprises determining that the patient following the administering step has a CMAI aggressive behavior score that is 4 to 6 less than the CMAI aggressive behavior score prior to the administering step and optionally a CMAI physically nonaggressive behavior score that is at least 1 less, 2 less, 3 less, 4 less, 5 less, 6 less, or 7 less than the CMAI physically nonaggressive behavior score prior to the administering step

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI physically nonaggressive behavior score following the administering step is determined 3 weeks after the administering step.

In some aspects of the foregoing embodiments, the CMAI physically nonaggressive behavior score following the administering step is determined 6 weeks after the administering step.

In some aspects of the foregoing embodiments, the CMAI physically nonaggressive behavior score following the administering step is determined 9 weeks after the administering step.

In some aspects of the foregoing embodiments, the CMAI physically nonaggressive behavior score following the administering step is determined 12 weeks after the administering step.

In some aspects of the foregoing embodiments, the CMAI aggressive behavior score following the administering step is determined 3 weeks after the administering step.

In some aspects of the foregoing embodiments, the CMAI aggressive behavior score following the administering step is determined 6 weeks after the administering step.

In some aspects of the foregoing embodiments, the CMAI aggressive behavior score following the administering step is determined 9 weeks after the administering step.

In some aspects of the foregoing embodiments, the CMAI aggressive behavior score following the administering step is determined 12 weeks after the administering step.

In some aspects of the foregoing embodiments, the CMAI aggressive behavior score determined 1 week after the administering step is 1 to 3 less, such as 2 less, such as 2.3 less than the CMAI aggressive behavior score prior to the administering step.

In some aspects of the foregoing embodiments, the CMAI aggressive behavior score determined 2 weeks after the administering step is 2 to 4 less, such as 3 less, such as 3.1 less than the CMAI aggressive behavior score prior to the administering step.

In some aspects of the foregoing embodiments, the CMAI aggressive behavior score determined 3 weeks after the administering step is 3 to 5 less, such as 4 less, such as 3.9 less than the CMAI aggressive behavior score prior to the administering step.

In some aspects of the foregoing embodiments, the CMAI aggressive behavior score determined 6 weeks after the administering step is 3 to 6 less, such as 4 to 5 less, such as 4 less, such as 4.0 less than the CMAI aggressive behavior score prior to the administering step.

In some aspects of the foregoing embodiments, the CMAI aggressive behavior score determined 9 weeks after the administering step is 3 to 6 less, such as 4 to 5 less, such as 5 less, such as 4.5 less than the CMAI aggressive behavior score prior to the administering step.

In some aspects of the foregoing embodiments, the CMAI aggressive behavior score determined 12 weeks after the administering step is 3 to 6 less, such as 4 to 5 less, such as 5 less, such as 5.1 less than the CMAI aggressive behavior score prior to the administering step.

In some embodiments, provided herein is a method of treating agitation associated with Alzheimer's disease in a patient that has been diagnosed as having Alzheimer's disease, comprising:
(1) determining the CMAI physically nonaggressive behavior score in the patient prior to step (2);
(2) administering to the patient therapeutically effective amounts of d6-DM and quinidine sulfate; and
(3) determining that the difference between the CMAI physically nonaggressive behavior score in the patient prior to the administering step and the CMAI physically nonaggressive behavior score in the patient following the administering step is from 3 to 7.

In some embodiments, the CMAI physically nonaggressive behavior score in the patient following the administering step is from 3 to 6 less, such as from 4 to 6 less, such as from 4.8 to 5.5 less, than the CMAI physically nonaggressive behavior score in the patient prior to the administering step.

In some embodiments, the subject prior to the administering step has one or more of:
i) a CMAI total score equal to the mean value±the standard deviation (SD) shown in Table 16 for AVP 786-18;
ii) a CMAI aggressive behavior score equal to the mean value±the standard deviation (SD) shown in Table 16 for AVP 786-18;
iii) a CMAI physically nonaggressive behavior score equal to the mean value±the standard deviation (SD) shown in Table 16 for AVP 786-18; or
iv) a CMAI verbal agitation score equal to the mean values±the standard deviation (SD) shown in Table 16 for AVP 786-18.

In some more particular embodiments, the method comprises determining that the patient has a CMAI physically nonaggressive behavior score of 12 to 31, such as at least 17, prior to the administering step.

In some more particular embodiments, the method comprises determining that the patient has a CMAI total score of 49 to 96, such as 72, prior to the administering step.

In some more particular embodiments, the method comprises determining that the patient has a CMAI physically nonaggressive behavior score of 12 to 31, such as at least 17, prior to the administering step and a reduction in CMAI total score of from 11 to 16 following the administering step.

In some more particular embodiments, the method comprises determining that the patient following the administering step has a CMAI physically nonaggressive behavior score that differs from the CMAI physically nonaggressive behavior score prior to the administering step by 3 to 7 and optionally a CMAI aggressive behavior score that differs from the CMAI aggressive behavior score prior to the administering step by no more than 7, 6, 5, 4, 3, 2, or 1.

In some more particular embodiments, the method comprises determining that the patient following the administering step has a CMAI physically nonaggressive behavior score that is 3 to 7 less than the CMAI physically nonaggressive behavior score prior to the administering step and optionally a CMAI aggressive behavior score that is at least 1 less, 2 less, 3 less, 4 less, 5 less, 6 less, or 7 less than the CMAI aggressive behavior score prior to the administering step

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI physically nonaggressive behavior score following the administering step is determined 3 weeks after the administering step.

In some aspects of the foregoing embodiments, the CMAI physically nonaggressive behavior score following the administering step is determined 6 weeks after the administering step.

In some aspects of the foregoing embodiments, the CMAI physically nonaggressive behavior score following the administering step is determined 9 weeks after the administering step.

In some aspects of the foregoing embodiments, the CMAI physically nonaggressive behavior score following the administering step is determined 12 weeks after the administering step.

In some aspects of the foregoing embodiments, the CMAI aggressive behavior score following the administering step is determined 3 weeks after the administering step.

In some aspects of the foregoing embodiments, the CMAI aggressive behavior score following the administering step is determined 6 weeks after the administering step.

In some aspects of the foregoing embodiments, the CMAI aggressive behavior score following the administering step is determined 9 weeks after the administering step.

In some aspects of the foregoing embodiments, the CMAI aggressive behavior score following the administering step is determined 12 weeks after the administering step.

In some aspects of the foregoing embodiments, the CMAI physically nonaggressive behavior score determined 1 week after the administering step is 1 to 3 less, such as 2 less, such as 2.3 less than the CMAI physically nonaggressive behavior score prior to the administering step.

In some aspects of the foregoing embodiments, the CMAI physically nonaggressive behavior score determined 2 weeks after the administering step is 2 to 4 less, such as 3 less, such as 3.0 less than the CMAI physically nonaggressive behavior score prior to the administering step.

In some aspects of the foregoing embodiments, the CMAI physically nonaggressive behavior score determined 3 weeks after the administering step is 3 to 5 less, such as 4 less, such as 3.8 less than the CMAI physically nonaggressive behavior score prior to the administering step.

In some aspects of the foregoing embodiments, the CMAI physically nonaggressive behavior score determined 6 weeks after the administering step is 3 to 6 less, such as 4 to 5 less, such as 5 less, such as 4.8 less than the CMAI physically nonaggressive behavior score prior to the administering step.

In some aspects of the foregoing embodiments, the CMAI physically nonaggressive behavior score determined 9 weeks after the administering step is 3 to 7 less, such as 4 to 6 less, such as 5 less, such as 4.9 less than the CMAI physically nonaggressive behavior score prior to the administering step.

In some aspects of the foregoing embodiments, the CMAI physically nonaggressive behavior score determined 12 weeks after the administering step is 3 to 7 less, such as 4 to 6 less, such as 6 less, such as 5.5 less than the CMAI physically nonaggressive behavior score prior to the administering step.

A method of treating agitation associated with Alzheimer's disease in a patient that has been diagnosed as having Alzheimer's disease, comprising:
(1) determining the CMAI physically nonaggressive behavior score in the patient prior to step (2);
(2) administering to the patient therapeutically effective amounts of d6-DM and quinidine sulfate; and
(3) determining that the difference between the CMAI physically nonaggressive behavior score in the patient prior to the administering step and the CMAI physically nonaggressive behavior score in the patient following the administering step is from 5 to 7.

In some embodiments, the CMAI physically nonaggressive behavior score in the patient following the administering step is from 5 to 6 less, such as 5.8 less, than the CMAI physically nonaggressive behavior score in the patient prior to the administering step.

In some more particular embodiments, the method comprises determining that the patient has a CMAI physically nonaggressive behavior score of 12 to 29, such as at least 17, prior to the administering step.

In some more particular embodiments, the method comprises determining that the patient has a CMAI total score of 50 to 92, such as 71, prior to the administering step.

In some more particular embodiments, the method comprises determining that the patient has a CMAI physically nonaggressive behavior score of 12 to 29, such as at least 17, prior to the administering step and a reduction in CMAI total score from 13 to 22 following the administering step.

In some more particular embodiments, the method comprises determining that the patient following the administering step has a CMAI physically nonaggressive behavior score that differs from the CMAI physically nonaggressive behavior score prior to the administering step by 5 to 6 and optionally a CMAI aggressive behavior score that differs from the CMAI aggressive behavior score prior to the administering step by no more than 7, 6, 5, 4, 3, 2, or 1.

In some more particular embodiments, the method comprises determining that the patient following the administering step has a CMAI physically nonaggressive behavior score that is 5 to 6 less than the CMAI physically nonaggressive behavior score prior to the administering step and optionally a CMAI aggressive behavior score that is at least 1 less, 2 less, 3 less, 4 less, 5 less, 6 less, or 7 less than the CMAI aggressive behavior score prior to the administering step

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI physically nonaggressive behavior score following the administering step is determined 3 weeks after the administering step.

In some aspects of the foregoing embodiments, the CMAI physically nonaggressive behavior score following the administering step is determined 6 weeks after the administering step.

In some aspects of the foregoing embodiments, the CMAI physically nonaggressive behavior score following the administering step is determined 9 weeks after the administering step.

In some aspects of the foregoing embodiments, the CMAI physically nonaggressive behavior score following the administering step is determined 12 weeks after the administering step.

In some aspects of the foregoing embodiments, the CMAI aggressive behavior score following the administering step is determined 3 weeks after the administering step.

In some aspects of the foregoing embodiments, the CMAI aggressive behavior score following the administering step is determined 6 weeks after the administering step.

In some aspects of the foregoing embodiments, the CMAI aggressive behavior score following the administering step is determined 9 weeks after the administering step.

In some aspects of the foregoing embodiments, the CMAI aggressive behavior score following the administering step is determined 12 weeks after the administering step.

In some aspects of the foregoing embodiments, the CMAI physically nonaggressive behavior score determined 1 week after the administering step is 1 to 3 less, such as 2 less, such as 2.3 less than the CMAI physically nonaggressive behavior score prior to the administering step.

In some aspects of the foregoing embodiments, the CMAI physically nonaggressive behavior score determined 2 weeks after the administering step is 2 to 4 less, such as 3 less, such as 3.1 less than the CMAI physically nonaggressive behavior score prior to the administering step.

In some aspects of the foregoing embodiments, the CMAI physically nonaggressive behavior score determined 3 weeks after the administering step is 3 to 5 less, such as 4 less, such as 3.7 less than the CMAI physically nonaggressive behavior score prior to the administering step.

In some aspects of the foregoing embodiments, the CMAI physically nonaggressive behavior score determined 6 weeks after the administering step is 3 to 6 less, such as 4 to 5 less, such as 4 less, such as 4.4 less than the CMAI physically nonaggressive behavior score prior to the administering step.

In some aspects of the foregoing embodiments, the CMAI physically nonaggressive behavior score determined 9 weeks after the administering step is 3 to 7 less, such as 4 to 6 less, such as 5.6 less than the CMAI physically nonaggressive behavior score prior to the administering step.

In some aspects of the foregoing embodiments, the CMAI physically nonaggressive behavior score determined 12 weeks after the administering step is 3 to 7 less, such as 4 to 6 less, such as 6 less, such as 5.8 less than the CMAI physically nonaggressive behavior score prior to the administering step.

A method of treating agitation associated with Alzheimer's disease in a patient that has been diagnosed as having Alzheimer's disease, comprising:
(1) determining the CMAI verbally agitated behavior score in the patient prior to step (2);
(2) administering to the patient therapeutically effective amounts of d6-DM and quinidine sulfate; and
(3) determining that the difference between the CMAI verbally agitated behavior score in the patient prior to the administering step and the CMAI verbally agitated behavior score in the patient following the administering step is from 3 to 5.

In some embodiments, the CMAI verbally agitated behavior score in the patient following the administering step is from 4 to 5 less, or from 3 to 4 less, such as 3.0 to 3.4 less, than the CMAI verbally agitated behavior score in the patient prior to the administering step.

In some embodiments, the subject prior to the administering step has one or more of: i) a CMAI total score equal to the mean value±the standard deviation (SD) shown in Table 16 for AVP 786-18;
ii) a CMAI aggressive behavior score equal to the mean value t the standard deviation (SD) shown in Table 16 for AVP 786-18;
iii) a CMAI physically nonaggressive behavior score equal to the mean value±the standard deviation (SD) shown in Table 16 for AVP 786-18; or
iv) a CMAI verbal agitation score equal to the mean values±the standard deviation (SD) shown in Table 16 for AVP 786-18.

In some more particular embodiments, the method comprises determining that the patient has a CMAI verbally agitated behavior score of 10 to 23, such as at least 19, prior to the administering step.

In some more particular embodiments, the method comprises determining that the patient has a CMAI total score of 49 to 96, such as 72, prior to the administering step.

In some more particular embodiments, the method comprises determining that the patient has a CMAI verbally agitated behavior score of 10 to 23, such as at least 19, prior to the administering step and a reduction in CMAI total score of from 11 to 16 following the administering step.

In some more particular embodiments, the method comprises determining that the patient following the administering step has a CMAI verbally agitated behavior score that differs from the CMAI verbally agitated behavior score prior to the administering step by 3 to 4 and optionally a CMAI aggressive behavior score that differs from the CMAI aggressive behavior score prior to the administering step by no more than 7, 6, 5, 4, 3, 2, or 1.

In some more particular embodiments, the method comprises determining that the patient following the administering step has a CMAI verbally agitated behavior score that is 3 to 4 less than the CMAI verbally agitated behavior score prior to the administering step and optionally a CMAI aggressive behavior score that is at least 1 less, 2 less, 3 less, 4 less, 5 less, 6 less, or 7 less than the CMAI aggressive behavior score prior to the administering step

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI verbally agitated behavior score following the administering step is determined 3 weeks after the administering step.

In some aspects of the foregoing embodiments, the CMAI verbally agitated behavior score following the administering step is determined 6 weeks after the administering step.

In some aspects of the foregoing embodiments, the CMAI verbally agitated behavior score following the administering step is determined 9 weeks after the administering step.

In some aspects of the foregoing embodiments, the CMAI verbally agitated behavior score following the administering step is determined 12 weeks after the administering step.

In some aspects of the foregoing embodiments, the CMAI aggressive behavior score following the administering step is determined 3 weeks after the administering step.

In some aspects of the foregoing embodiments, the CMAI aggressive behavior score following the administering step is determined 6 weeks after the administering step.

In some aspects of the foregoing embodiments, the CMAI aggressive behavior score following the administering step is determined 9 weeks after the administering step.

In some aspects of the foregoing embodiments, the CMAI aggressive behavior score following the administering step is determined 12 weeks after the administering step.

In some aspects of the foregoing embodiments, the CMAI verbally agitated behavior score determined 1 week after the administering step is 1 to 2 less, such as 1 less, such as 1.2 less than the CMAI verbally agitated behavior score prior to the administering step.

In some aspects of the foregoing embodiments, the CMAI verbally agitated behavior score determined 2 weeks after the administering step is 1 to 3 less, such as 2 less, such as 2.0 less than the CMAI verbally agitated behavior score prior to the administering step.

In some aspects of the foregoing embodiments, the CMAI verbally agitated behavior score determined 3 weeks after the administering step is 1 to 4 less, such as 2 to 3 less, such as 2.5 less than the CMAI verbally agitated behavior score prior to the administering step.

In some aspects of the foregoing embodiments, the CMAI verbally agitated behavior score determined 6 weeks after the administering step is 2 to 4 less, such as 3 less, such as 3.0 less than the CMAI verbally agitated behavior score prior to the administering step.

In some aspects of the foregoing embodiments, the CMAI verbally agitated behavior score determined 9 weeks after the administering step is 2 to 4 less, such as 3 less, such as 3.2 less than the CMAI verbally agitated behavior score prior to the administering step.

In some aspects of the foregoing embodiments, the CMAI verbally agitated behavior score determined 12 weeks after the administering step is 2 to 4 less, such as 3 less, such as 3.4 less than the CMAI verbally agitated behavior score prior to the administering step.

A method of treating agitation associated with Alzheimer's disease in a patient that has been diagnosed as having Alzheimer's disease, comprising:
(1) determining the CMAI verbally agitated behavior score in the patient prior to step (2);
(2) administering to the patient therapeutically effective amounts of d6-DM and quinidine sulfate; and
(3) determining that the difference between the CMAI verbally agitated behavior score in the patient prior to the administering step and the CMAI verbally agitated behavior score in the patient following the administering step is from 4 to 6.

In some embodiments, the CMAI verbally agitated behavior score in the patient following the administering step is from 4 to 5 less, or from 5 to 6 less, such as 5 to 5.5 such as 5.2 less, than the CMAI verbally agitated behavior score in the patient prior to the administering step.

In some more particular embodiments, the method comprises determining that the patient has a CMAI verbally agitated behavior score of 11 to 23, such as at least 19, prior to the administering step.

In some more particular embodiments, the method comprises determining that the patient has a CMAI total score of 50 to 92, such as 71, prior to the administering step.

In some more particular embodiments, the method comprises determining that the patient has a CMAI verbally agitated behavior score of 11 to 23, such as at least 19, prior to the administering step and a reduction in CMAI total score from 13 to 22 following the administering step.

In some more particular embodiments, the method comprises determining that the patient following the administering step has a CMAI verbally agitated behavior score that differs from the CMAI verbally agitated behavior score prior to the administering step by 4 to 6 and optionally a CMAI aggressive behavior score that differs from the CMAI aggressive behavior score prior to the administering step by no more than 7, 6, 5, 4, 3, 2, or 1.

In some more particular embodiments, the method comprises determining that the patient following the administering step has a CMAI verbally agitated behavior score that is 4 to 6 less than the CMAI verbally agitated behavior score prior to the administering step and optionally a CMAI aggressive behavior score that is at least 1 less, 2 less, 3 less, 4 less, 5 less, 6 less, or 7 less than the CMAI aggressive behavior score prior to the administering step

In some aspects of the foregoing embodiments, the CMAI total score following the administering step is determined 3 weeks after the administering step.

In some aspects of the foregoing embodiments, the CMAI total score following the administering step is determined 6 weeks after the administering step.

In some aspects of the foregoing embodiments, the CMAI total score following the administering step is determined 9 weeks after the administering step.

In some aspects of the foregoing embodiments, the CMAI total score following the administering step is determined 12 weeks after the administering step.

In some aspects of the foregoing embodiments, the CMAI verbally agitated behavior score following the administering step is determined 3 weeks after the administering step.

In some aspects of the foregoing embodiments, the CMAI verbally agitated behavior score following the administering step is determined 6 weeks after the administering step.

In some aspects of the foregoing embodiments, the CMAI verbally agitated behavior score following the administering step is determined 9 weeks after the administering step.

In some aspects of the foregoing embodiments, the CMAI verbally agitated behavior score following the administering step is determined 12 weeks after the administering step.

In some aspects of the foregoing embodiments, the CMAI aggressive behavior score following the administering step is determined 3 weeks after the administering step.

In some aspects of the foregoing embodiments, the CMAI aggressive behavior score following the administering step is determined 6 weeks after the administering step.

In some aspects of the foregoing embodiments, the CMAI aggressive behavior score following the administering step is determined 9 weeks after the administering step.

In some aspects of the foregoing embodiments, the CMAI aggressive behavior score following the administering step is determined 12 weeks after the administering step.

In some aspects of the foregoing embodiments, the CMAI verbally agitated behavior score determined 1 week after the administering step is 1 to 3 less, such as 2 less, such as 1.8 less than the CMAI verbally agitated behavior score prior to the administering step.

In some aspects of the foregoing embodiments, the CMAI verbally agitated behavior score determined 2 weeks after the administering step is 2 to 4 less, such as 3 less, such as 2.7 less than the CMAI verbally agitated behavior score prior to the administering step.

In some aspects of the foregoing embodiments, the CMAI verbally agitated behavior score determined 3 weeks after the administering step is 2 to 4 less, such as 3 less, such as 2.7 less than the CMAI verbally agitated behavior score prior to the administering step.

In some aspects of the foregoing embodiments, the CMAI verbally agitated behavior score determined 6 weeks after the administering step is 2 to 4 less, such as 3 less, such as 3.4 less than the CMAI verbally agitated behavior score prior to the administering step.

In some aspects of the foregoing embodiments, the CMAI verbally agitated behavior score determined 9 weeks after the administering step is 3 to 6 less, such as 4 to 5 less, such as 4.5 less than the CMAI verbally agitated behavior score prior to the administering step.

In some aspects of the foregoing embodiments, the CMAI verbally agitated behavior score determined 12 weeks after the administering step is 3 to 7 less, such as 4 to 6 less, such as 5 less, such as 5.2 less than the CMAI verbally agitated behavior score prior to the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI-AA score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI-AA score in the patient prior to said administration, wherein the patient has been assessed as having a NPI-AA score of greater than or equal to 4 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments of a method disclosed herein, the method comprises determining the NPI total score in the patient prior to the administering step.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI total score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI total score in the patient prior to said administration, wherein the patient has been assessed as having a NPI total score of greater than or equal to 2 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments of a method disclosed herein, the method comprises determining the NPI Aberrant Motor Behavior domain score in the patient prior to the administering step.

In some embodiments the NPI Aberrant Motor Behavior domain score prior to the administering step is equal to or greater than 2.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI Aberrant Motor Behavior domain score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI Aberrant Motor Behavior domain score in the patient prior to said administration, wherein the patient has been assessed as having a NPI Aberrant Motor Behavior domain score of greater than or equal to 2 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments of a method disclosed herein, the method comprises determining the NPI Irritability/Lability domain score in the patient prior to the administering step.

In some embodiments the NPI Irritability/Lability domain score prior to the administering step is equal to or greater than 2.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI Irritability/Lability domain score in the patient prior to said administration, wherein the patient has been assessed as having a Cohen-Mansfield agitation inventory (CMAI) total score of greater than or equal to 33, such as 33 to 203, such as 45 to 120, such as 55 to 90 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, the present disclosure provides a method of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate, wherein the method comprises determining the CMAI total score and the NPI Irritability/Lability domain score in the patient prior to said administration, wherein the patient has been assessed as having a NPI Irritability/Lability domain score of greater than or equal to 2 prior to the administering step and the method comprises determining the CMAI total score in the patient following the administering step. In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the difference between the CMAI total score in the patient prior to the administering step and the CMAI total score in the patient following the administering step is at least 1, such as from 1 to 150, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40.

In some embodiments, provided herein is a method of treating agitation associated with Alzheimer's disease in a patient that has been diagnosed as having Alzheimer's disease, comprising:
determining that the patient is in a CMAI Factor 1 agitated status as defined herein;
administering to the patient therapeutically effective amounts of d6-DM and quinidine sulfate; and
determining that, following the administering step, the patient is in a CMAI Factor 1 not agitated status as defined herein.

In some embodiments, provided herein is a method of treating agitation associated with Alzheimer's disease in a patient that has been diagnosed as having Alzheimer's disease, comprising:
determining that the patient is in a CMAI Factor 2 agitated status as defined herein;
administering to the patient therapeutically effective amounts of d6-DM and quinidine sulfate; and
determining that, following the administering step, the patient is in a CMAI Factor 2 not agitated status as defined herein.

In some embodiments, provided herein is a method of treating agitation associated with Alzheimer's disease in a patient that has been diagnosed as having Alzheimer's disease, comprising:
determining that the patient is in a CMAI Factor 3 agitated status as defined herein;
administering to the patient therapeutically effective amounts of d6-DM and quinidine sulfate; and
determining that, following the administering step, the patient is in a CMAI Factor 3 not agitated status as defined herein.

In some embodiments of a method disclosed herein, the method comprises determining the MMSE score in the patient prior to the administering step.

In some embodiments the MMSE score prior to the administering step is 4 to 30.

In some embodiments the MMSE score prior to the administering step is 8 to 24.

In some embodiments the MMSE score prior to the administering step is from 6 to 26.

In some embodiments, provided herein is a method of treating agitation associated with Alzheimer's disease in a patient that has been diagnosed as having Alzheimer's disease, comprising administering to the patient therapeutically effective amounts of d6-DM and quinidine sulfate,

wherein the patient has been diagnosed as having an MMSE score from 4 to 28 prior to the administering step.

In some embodiments, provided herein is a method of treating agitation associated with Alzheimer's disease in a patient that has been diagnosed as having Alzheimer's disease, comprising:
(1) determining the CMAI total score in the patient prior to step (2);
(2) administering to the patient therapeutically effective amounts of d6-DM and quinidine sulfate; and
(3) determining that the difference between the CMAI total score in the patient prior to the administering step and the CMAI total score in the patient following the administering step is from 11 to 16,
wherein the patient has been diagnosed as having an MMSE score from 4 to 28 prior to the administering step.

In some embodiments, the CMAI total score following the administering step is from 12 to 15 less, such as from 13 to 15 less, such as 14.3 less than the CMAI total score prior to the administering step.

In some embodiments the patient has been diagnosed as having an MMSE score from 6 to 26 prior to the administering step.

In some embodiments the patient has been diagnosed as having an MMSE score from 8 to 24 prior to the administering step.

In some embodiments the patient has been diagnosed as having an MMSE score from 10 to 22 prior to the administering step.

In some embodiments the patient has been diagnosed as having an MMSE score from 10 to 22 prior to the administering step.

In some embodiments of a method disclosed herein, the method comprises determining the CGIS-Agitation score in the patient prior to the administering step.

In some embodiments of a method disclosed herein, the CGIS-Agitation score in the patient prior to the administering step is greater than or equal to 2.

In some embodiments of a method disclosed herein, the CGIS-Agitation score in the patient prior to the administering step is greater than or equal to 3.

In some embodiments of a method disclosed herein, the CGIS-Agitation score in the patient prior to the administering step is greater than or equal to 4.

In some embodiments, the CGIS-Agitation score in the patient following the administering step is at least 15% lower, such as at least 50% lower, than the CGIS-Agitation score in the patient prior to the administering step.

In some embodiments of a method disclosed herein, the method comprises determining the mADCS-CGIC-Agitation score in the patient following the administering step.

In some more particular embodiments, the mADCS-CGIC-Agitation score is ≤2 following the administering step.

In some more particular embodiments, the mADCS-CGIC-Agitation score is ≤3 following the administering step.

In some more particular embodiments, the mADCS-CGIC-Agitation score is ≤4 following the administering step.

In some embodiments of a method disclosed herein, the method comprises determining the PGIC score in the patient prior to the administering step.

In some more particular embodiments, the PGIC score is ≤2 following the administering step.

In some more particular embodiments, the PGIC score is ≤3 following the administering step.

In some more particular embodiments, the PGIC score is ≤4 following the administering step.

In some embodiments of a method disclosed herein, the patient has been treated or is being treated with an atypical antipsychotic other than clozapine prior to the administering step.

In some embodiments of a method disclosed herein, the patient has been treated or is being treated with an antidepressant other than nefazodone, a tricyclic antidepressant, or a monoamine oxidase inhibitors (MAOI) prior to the administering step.

In some embodiments of a method disclosed herein, the patient has been treated or is being treated with memantine prior to the administering step.

In some embodiments of a method disclosed herein, the patient has been treated or is being treated with an acetylcholinesterase inhibitor, such as donepezil, prior to the administering step.

In some embodiments of a method disclosed herein, the patient has been treated or is being treated with memantine, and the patient has not been treated and is not being treated with an acetylcholinesterase inhibitor, such as donepezil, prior to the administering step.

In some embodiments of a method disclosed herein, the patient has been treated or is being treated with an acetylcholinesterase inhibitor, such as donepezil, and the patient has not been treated and is not being treated with memantine, prior to the administering step.

In some embodiments of a method disclosed herein, the patient has been treated or is being treated with memantine, and the patient has not been treated and is not being treated with an acetylcholinesterase inhibitor, such as donepezil, prior to the administering step.

In some embodiments of a method disclosed herein, the patient has been treated or is being treated with an acetylcholinesterase inhibitor, such as donepezil, and the patient has not been treated and is not being treated with memantine, prior to the administering step.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a Cohen-Mansfield agitation inventory (CMAI) total score greater than or equal to 33, such as 33 to 203, such as 45 to 120, such as 55 to 90 prior to the administering step, wherein the patient is not being treated with one or more monoamine oxidase inhibitors (MAOIs).

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a Cohen-Mansfield agitation inventory (CMAI) total score greater than or equal to 33, such as 33 to 203, such as 45 to 120, such as 55 to 90 prior to the administering step, wherein the patient is not being treated with clozapine.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a Cohen-Mansfield agitation inventory (CMAI) total score greater than or equal to 33, such as 33 to 203, such as 45 to 120, such as 55 to 90 prior to the administering step. wherein the patient is not being treated with an agent that:

(a) increases levels of quinidine sulfate;
(b) is metabolized by CYP2D6;
(c) is related to quinidine sulfate;
(d) produces serotonin syndrome when co-administered with d6-DM;
(e) decreases plasma levels of d6-DM and quinidine sulfate;
(f) is clozapine,
(g) is a typical antipsychotic,
(h) is nefazodone;
(i) is a tricyclic antidepressant;
(j) is a monoamine oxidase inhibitors (MAOI);
(k) is a benzodiazepine,
(l) is a typical antipsychotic; or
(m) is selected from the group consisting of atomoxetine, carbamazepine, fosphenytoin, pentobarbital, phenobarbital, phenytoin, and primidone.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient does not have a suicide risk. In some embodiments, suicide risk is determined by one or more of the following:

(a) judgment of the prescribing doctor;
(b) the patient answers yes on the Columbia Suicide Severity Rating Scale (C-SSRS Suicidal Ideation Item 4 (active suicidal ideation with some intent to act, without a specific plan) and the patient's most recent episode meeting this C-SSRS Item 4 occurred within six months;
(c) the patient answers yes on the C-SSRS Suicidal Behavior Item 5 (active suicidal ideation with specific plan and intent) and the patient's most recent episode meeting this C-SSRS Item 5 occurred within six months; or
(d) the patient answers yes on any of the 5 on the C-SSRS Suicidal Behavior Items (active attempt, interrupted attempt, aborted attempt, preparatory acts, or behavior) and the patient's most recent episode meeting any of these C-SSRS Items occurred within two years prior to treatment.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient does not have a cardiovascular history of any one or more of:
(a) history or evidence of complete heart block, ventricular tachycardia, presence of clinically significant premature ventricular contractions (PVCs) as evaluated by a central reader, QTc prolongation, or torsades de pointes;
(b) QTc using the Fridericia's formula (QTcF) greater than 450 msec for males and greater than 470 msec for females based on central review, unless due to ventricular pacing;
(c) family history of congenital QT interval prolongation syndrome; or
(d) history or presence of clinically significant syncope, orthostatic hypotension, or postural tachycardia.

In some embodiments, provided herein is a method of treating agitation associated with Alzheimer's disease in a patient that has been diagnosed as having Alzheimer's disease, comprising administering to the patient therapeutically effective amounts of d6-DM and quinidine sulfate, wherein the patient is not a male patient with a QTcF interval of >450 msec or a female patient with a QTcF interval of >470 msec.

In some embodiments, provided herein is a method of treating agitation associated with Alzheimer's disease in a patient that has been diagnosed as having Alzheimer's disease, comprising administering to the patient therapeutically effective amounts of d6-DM and quinidine sulfate, wherein the patient is not a male patient with a QTcF interval of >450 msec that is not due to ventricular pacing, or a female patient with a QTcF interval of >470 msec that is not due to ventricular pacing.

In some embodiments, provided herein is a method of treating agitation associated with Alzheimer's disease in a patient that has been diagnosed as having Alzheimer's disease, comprising:
determining that the patient is not a male patient with a QTcF interval of >450 msec or a female patient with a QTcF interval of >470 msec; and
administering to the patient therapeutically effective amounts of d6-DM and quinidine sulfate.

In some embodiments, provided herein is a method of treating agitation associated with Alzheimer's disease in a patient that has been diagnosed as having Alzheimer's disease, comprising:
determining that the patient is not a male patient with a QTcF interval of >450 msec that is not due to ventricular pacing, or a female patient with a QTcF interval of >470 msec that is not due to ventricular pacing; and
administering to the patient therapeutically effective amounts of d6-DM and quinidine sulfate.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient does not have Parkinson's disease.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the Cohen-Mansfield agitation inventory (CMAI) total score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a Cohen-Mansfield agitation inventory (CMAI) total score greater than or equal to 33, such as 33 to 203, such as 45 to 120, such as 55 to 90 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the Cohen-Mansfield agitation inventory (CMAI) total score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to greater than or equal to 33, such as 33 to 203, such as 45 to 120, such as 55 to 90 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

This disclosure provides, in some embodiments, methods of treating aggressive agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating aggressive agitation associated with Alzheimer's disease in a subject, comprising administering to the subject therapeutically effective amounts of deuterated [d6]-dextromethorphan hydrobromide (d6-DM) and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating aggressive agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating aggressive agitation associated with Alzheimer's disease in a subject, comprising administering to the subject a therapeutically effective amount of a composition comprising deuterated [d6]-dextromethorphan hydrobromide (d6-DM) and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating aggressive agitation associated with Alzheimer's disease in a patient having Alzheimer's disease, comprising administering to the patient therapeutically effective amounts of d6-DM and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI aggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating aggressive agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI aggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

This disclosure provides, in some embodiments, methods of treating physically nonaggressive agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating physically nonaggressive agitation associated with Alzheimer's disease in a subject, comprising administering to the subject therapeutically effective amounts of deuterated [d6]-dextromethorphan hydrobromide (d6-DM) and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating physically nonaggressive agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating physically nonaggressive agitation associated with Alzheimer's disease in a subject, comprising administering to the subject a therapeutically effective amount of a composition comprising deuterated [d6]-dextromethorphan hydrobromide (d6-DM) and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine:
d) an acetylcholinesterase inhibitor, such as donepezil; or
e) a combination of one or more of the foregoing.

In some embodiments, the present disclosure provides a method of treating physically nonaggressive agitation associated with Alzheimer's disease in a patient having Alzheimer's disease, comprising administering to the patient therapeutically effective amounts of d6-DM and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI physically nonaggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating physically nonaggressive agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration,

wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating physically nonaggressive agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI physically nonaggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

This disclosure provides, in some embodiments, methods of treating verbal agitation associated with Alzheimer's disease in a subject, comprising administering to the subject therapeutically effective amounts of deuterated [d6]-dextromethorphan, or a salt thereof, and quinidine, or a salt thereof. In some embodiments, provided herein are methods of treating verbal agitation associated with Alzheimer's disease in a subject, comprising administering to the subject therapeutically effective amounts of deuterated [d6]-dextromethorphan hydrobromide (d6-DM) and quinidine sulfate, wherein prior to the administering step the patient has been treated or is being treated with.
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing. In some embodiments the deuterated [d6]-dextromethorphan hydrobromide is a deuterated [d6]-dextromethorphan hydrobromide hydrate, such as [d6]-dextromethorphan hydrobromide monohydrate.

This disclosure provides, in some embodiments, methods of treating verbal agitation associated with Alzheimer's disease in a subject, comprising administering to the subject a therapeutically effective amount of a composition comprising deuterated [d6]-dextromethorphan, or a salt thereof, and quinidine, or a salt thereof. In some embodiments, provided herein are methods of treating verbal agitation associated with Alzheimer's disease in a subject, comprising administering to the subject a therapeutically effective amount of a composition comprising deuterated [d6]-dextromethorphan hydrobromide (d6-DM) and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating verbal agitation associated with Alzheimer's disease in a patient having Alzheimer's disease, comprising administering to the patient therapeutically effective amounts of d6-DM and quinidine sulfate, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the CMAI verbally agitated behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating verbal agitation associated with Alzheimer's disease in a patient that has been diagnosed as having Alzheimer's disease, comprising administering to the patient therapeutically effective amounts of d6-DM and quinidine sulfate, wherein the method comprises determining the CMAI total score in the patient prior to said administration, wherein prior to the administering step the patient has been treated or is being treated with.
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating verbal agitation associated with Alzheimer's disease in a patient having Alzheimer's disease, comprising administering to the patient therapeutically effective amounts of d6-DM and quinidine sulfate, wherein the method comprises determining the CMAI total score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CMAI verbally agitated behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI aggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI aggressive behavior score of CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some more particular embodiments, the method comprises determining the score in the patient prior to the administering step for at least one of the following CMAI aggressive behavior items:
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; or
12) cursing or verbal aggression.

In some more particular embodiments, the patient has been assessed to have a score of 2 or greater for at least one of CMAI aggressive behavior items 1) to 12) hereinabove.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI physically nonaggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some more particular embodiments, the method comprises determining the score in the patient prior to the administering step for at least one of the following CMAI physically nonaggressive behavior items (assessed according to the method described elsewhere herein for assessing the CMAI total score according to the CMAI Manual, except that the score based on frequency of occurrence only needs to be determined for the at least one of the CMAI physically nonaggressive behavior items according to the methods of these embodiments):
1) pacing and/or aimless wandering
2) trying to get to a different place;
3) general restlessness;
4) inappropriate dressing or disrobing;
5) handling things inappropriately;
or
6) performing repetitious mannerisms

In some more particular embodiments, the patient has been assessed to have a score of 2 or greater for at least one of CMAI physically nonaggressive behavior items 1) to 6) hereinabove.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CMAI verbally agitated behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI verbally agitated behavior score of greater than or equal to 8, such as 8 to 28, such as 12 to 25, such as 12, 13, 14, 15, 16, 17, 18, 19 20, 21, 22, 23, 24, or 25 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CMAI verbally agitated behavior score greater than or equal to 8, such as 8 to 28, such as 12 to 25, such as 12, 13, 14, 15, 16, 17, 18, 19 20, 21, 22, 23, 24, or 25, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some more particular embodiments, the method comprises determining the score in the patient prior to the administering step for at least one of the following CMAI verbally agitated behavior items (assessed according to the method described elsewhere herein for assessing the CMAI total score according to the CMAI Manual, except that the score based on frequency of occurrence only needs to be determined for the at least one of the CMAI verbally agitated behavior items according to the methods of these embodiments):
1) complaining;
2) constant unwarranted requests for attention and/or help;
3) repetitive sentences or questions; or
4) negativism.

In some more particular embodiments, the patient has been assessed to have a score of 2 or greater for at least one of CMAI verbally agitated behavior items 1) to 4) hereinabove.

In some embodiments of a method disclosed herein, the method comprises determining the CMAI total score in the patient following the administering step.

In some embodiments, the difference between the CMAI total score in the patient prior to the administering step and the CMAI total score in the patient following the administering step is at least 1, such as from 1 to 203, such as from 2 to 130, such as from 3 to 110, such as from 4 to 100, such as from 5 to 90, such as from 6 to 80, such as from 7 to 70, such as from 8 to 60, such as from 9 to 50, such as from 10 to 40

In some embodiments, the present disclosure provides a method of treating aggressive agitation associated with Alzheimer's disease in a patient that has been diagnosed as having Alzheimer's disease, comprising administering to the patient therapeutically effective amounts of d6-DM and quinidine sulfate, wherein the method comprises determining the NPI-AA score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the NPI-AA score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI-AA score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI-AA score in the patient and the CMAI physically nonaggressive behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10 and a CMAI physically nonaggressive behavior score of greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10 and a CMAI physically nonaggressive behavior score greater than or equal to 10, such as 10 to 42, such as 10 to 35, such as 10 to 30, such as 10, 11, 12, 13, 14, 15, 16, 17, 18, 19 20, 21, 22, 23, 24, 25, 26, 27, 28, 29, or 30, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the NPI-AA score in the patient and the CMAI verbally agitated behavior score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10 and a CMAI verbally agitated behavior score of greater than or equal to 8, such as 8 to 28, such as 12 to 25, such as 12, 13, 14, 15, 16, 17, 18, 19 20, 21, 22, 23, 24, or 25 prior to the administering step, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a NPI-AA score of greater than or equal to 3, such as 3 to 12, such as 3, 4, 5, 6, 7, 8, 9, or 10 and a CMAI verbally agitated behavior score of greater than or equal to 8, such as 8 to 28, such as 12 to 25, such as 12, 13, 14, 15, 16, 17, 18, 19 20, 21, 22, 23, 24, or 25, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments of a method disclosed herein, the method comprises determining the MMSE score in the patient prior to the administering step.

In some embodiments the MMSE score prior to the administering step is 4 to 30.

In some embodiments the MMSE score prior to the administering step is 8 to 24.

In some embodiments the MMSE score prior to the administering step is from 6 to 26.

In some embodiments the MMSE score prior to the administering step is equal to or greater than 17.

In some embodiments of a method disclosed herein, the method comprises determining the CGIS-Agitation score in the patient prior to the administering step.

In some embodiments of a method disclosed herein, the CGIS-Agitation score in the patient prior to the administering step is greater than or equal to 2

In some embodiments of a method disclosed herein, the CGIS-Agitation score in the patient prior to the administering step is greater than or equal to 3.

In some embodiments of a method disclosed herein, the CGIS-Agitation score in the patient prior to the administering step is greater than or equal to 4.

In some embodiments of a method disclosed herein, the method comprises determining the mADCS-CGIC-Agitation score in the patient following the administering step.

In some more particular embodiments, the mADCS-CGIC-Agitation score is ≤2 following the administering step.

In some more particular embodiments, the mADCS-CGIC-Agitation score is ≤3 following the administering step.

In some more particular embodiments, the mADCS-CGIC-Agitation score is ≤4 following the administering step.

In some embodiments of a method disclosed herein, the method comprises determining the PGIC score in the patient prior to the administering step.

In some more particular embodiments, the PGIC score is ≤2 following the administering step.

In some more particular embodiments, the PGIC score is ≤3 following the administering step.

In some more particular embodiments, the PGIC score is ≤4 following the administering step.

In some embodiments of a method disclosed herein, the patient has been treated or is being treated with an atypical antipsychotic other than clozapine prior to the administering step.

In some embodiments of a method disclosed herein, the patient has been treated or is being treated with an antidepressant other than nefazodone, a tricyclic antidepressant, or a monoamine oxidase inhibitors (MAOI) prior to the administering step.

In some embodiments of a method disclosed herein, the patient has been treated or is being treated with memantine prior to the administering step.

In some embodiments of a method disclosed herein, the patient has been treated or is being treated with an acetylcholinesterase inhibitor, such as donepezil, prior to the administering step.

In some embodiments of a method disclosed herein, the patient has been treated or is being treated with memantine, and the patient has not been treated and is not being treated with antipsychotics, prior to the administering step.

In some embodiments of a method disclosed herein, the patient has been treated or is being treated with an acetylcholinesterase inhibitor, such as donepezil, and the patient has not been treated and is not being treated with antipsychotics, prior to the administering step.

In some embodiments of a method disclosed herein, the patient has been treated or is being treated with memantine, and the patient has not been treated and is not being treated with an acetylcholinesterase inhibitor, such as donepezil, prior to the administering step.

In some embodiments of a method disclosed herein, the patient has been treated or is being treated with an acetylcholinesterase inhibitor, such as donepezil, and the patient has not been treated and is not being treated with memantine, prior to the administering step.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the method comprises determining the CGIS-Agitation score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the method comprises determining the CGIS-Agitation score in the patient prior to said administration, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CGIS-Agitation score of greater than or equal to 3, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

In some embodiments, the present disclosure provides a method of treating agitation associated with Alzheimer's disease in a patient having Alzheimer's disease, comprising administering to the patient therapeutically effective amounts of d6-DM and quinidine sulfate, wherein the patient has been assessed as having a CGIS-Agitation score of greater than or equal to 3, wherein prior to the administering step the patient has been treated or is being treated with:
a) an atypical antipsychotic other than clozapine;
b) an antidepressant other than nefazodone, a tricyclic antidepressant, or a monoamine oxidase inhibitor (MAOI);
c) memantine;
d) an acetylcholinesterase inhibitor, such as donepezil; or
e) a combination of one or more of the foregoing.

Unless otherwise specified, the doses described herein refer to the hydrobromide and sulfate salt forms of deuterated [d6]-dextromethorphan and quinidine, respectively. Based on such information, those skilled in the art can calculate corresponding dosages for the free-base forms of each active ingredient. A person of skill in the art can calculate the molecular weight for the salt of deuterated [d6]-dextromethorphan and the molecular weight for free base of deuterated [d6]-dextromethorphan and use the ratio to calculate appropriate dosages for the free base as well as for a salt. In some embodiments the deuterated [d6]-dextromethorphan hydrobromide is a deuterated [d6]-dextromethorphan hydrobromide hydrate, such as [d6]-dextromethorphan hydrobromide monohydrate.

The present disclosure provides, in some embodiments, methods of treating agitation associated with Alzheimer's disease in a patient that has been diagnosed as having Alzheimer's disease, comprising administering to the patient therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

The present disclosure provides, in some embodiments, methods of treating agitation associated with Alzheimer's disease in a patient having Alzheimer's disease, comprising administering to the patient therapeutically effective amounts of deuterated [d6]-dextromethorphan hydrobromide (d6-DM) and quinidine sulfate. In some embodiments the deuterated [d6]-dextromethorphan hydrobromide is a deuterated [d6]-dextromethorphan hydrobromide hydrate, such as [d6]-dextromethorphan hydrobromide monohydrate.

An exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the d6-DM is administered in a dose of from 14.4 mg to 22.5 mg twice daily and the quinidine sulfate is administered in a dose of from 3.9 mg to 6.1 mg dose twice daily. An exemplary embodiment of the present disclosure includes a method of treating agitation associated with Alzheimer's disease in a patient, comprising administering to the patient therapeutically effective amounts of d6-DM and quinidine sulfate, wherein the d6-DM is administered in dose of from 34.4 mg to 53.8 mg twice daily twice daily and the quinidine sulfate is administered in a from 3.9 mg to 6.1 mg dose twice daily.

An exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the d6-DM is administered in a dose of from 14.4 mg to 22.5 mg twice daily and the quinidine sulfate is administered in a 4.9 mg dose twice daily. An exemplary embodiment of the present disclosure includes a method of treating agitation associated with Alzheimer's disease in a patient, comprising administering to the patient therapeutically effective amounts of d6-DM and quinidine sulfate, wherein the d6-DM is administered in dose of from 34.4 mg to 53.8 mg twice daily twice daily and the quinidine sulfate is administered in a 4.9 mg dose twice daily.

In some embodiments, the d6-DM is administered in an 18 mg dose twice daily and the quinidine sulfate is administered in a 4.9 mg dose twice daily. In some embodiments, the d6-DM is administered in a 42.63 mg dose twice daily and the quinidine sulfate is administered in a 4.9 mg dose twice daily.

In some embodiments, provided herein is a pharmaceutical composition comprising 18 mg of d6-DM and 4.9 mg of quinidine sulfate. In some embodiments, provided herein is a pharmaceutical composition comprising 42.63 mg of d6-DM and 4.9 mg of quinidine sulfate.

In some embodiments, each capsule contains 42.63 mg of d6-DM and 4.9 mg of quinidine sulfate, and is administered twice daily.

In some embodiments of a method disclosed herein, the administration of one component (e.g., d6-DM) is concomitant with the administration of the other component (e.g., quinidine sulfate).

In some embodiments, the d6-DM is administered in a 14.4, mg, 18 mg, or 22.5 mg dose, e.g., once or twice daily, e.g., twice daily. In some embodiments, the d6-DM is administered in a 14.4 mg dose, e.g., once or twice daily, e.g., twice daily. In some embodiments, the d6-DM is administered in a 18 mg dose, e.g., once or twice daily, e.g., twice daily. In some embodiments, the d6-DM is administered in a 22.5 mg dose, e.g., once or twice daily, e.g., twice daily.

In some embodiments, the d6-DM is administered in a 34.4, mg, 42.63 mg, or 53.8 mg dose, e.g., once or twice daily, e.g., twice daily. In some embodiments, the d6-DM is administered in a 34.4 mg dose, e.g., once or twice daily, e.g., twice daily. In some embodiments, the d6-DM is administered in a 42.63 mg dose, e.g., once or twice daily, e.g., twice daily. In some embodiments, the d6-DM is administered in a 53.8 mg dose, e.g., once or twice daily, e.g., twice daily.

In some embodiments, the quinidine sulfate is administered in a 4.9 mg dose, e.g., once or twice daily, e.g., twice daily.

In some embodiments, the d6-DM and the quinidine sulfate are administered or used in a unit dosage form. In some embodiments, the unit dosage form includes 14.4, mg, 18 mg, or 22.5 mg of d6-DM and 4.9 mg of quinidine sulfate. In some embodiments, the unit dosage form includes 14.4 mg of d6-DM and 4.9 mg of quinidine sulfate. In some embodiments, the unit dosage form includes 18 mg of d6-DM and 4.9 mg of quinidine sulfate. In some embodiments, the unit dosage form includes 22.5 mg of d6-DM and 4.9 mg of quinidine sulfate. In some embodiments, the unit dosage form includes 34.4, mg, 42.63 mg, or 53.8 mg of d6-DM and 4.9 mg of quinidine sulfate. In some embodiments, the unit dosage form includes 34.4, mg of d6-DM and 4.9 mg of quinidine sulfate. In some embodiments, the unit dosage form includes 42.63 mg of d6-DM and 4.9 mg of quinidine sulfate. In some embodiments, the unit dosage form includes 53.8 mg of d6-DM and 4.9 mg of quinidine sulfate. In some embodiments, the unit dosage forms of the d6-DM and the quinidine sulfate are in the form of a tablet or a capsule. In some embodiments, the unit dosage forms of the d6-DM and the quinidine sulfate are in the form of a capsule. In some embodiments, the unit dosage forms of the d6-DM and the quinidine sulfate are in the form of a tablet.

In some embodiments, the d6-DM and the quinidine sulfate are administered or used in a combined dose, or in separate doses. In some embodiments, the separate doses are administered substantially concomitantly.

The present disclosure provides, in some embodiments, a medicament comprising a therapeutically effective amount of deuterated [d6]-dextromethorphan hydrobromide (d6-DM) for use in the treatment of agitation associated with Alzheimer's disease in a patient having Alzheimer's disease, which is used in combination with a therapeutically effective amount of quinidine sulfate (Q) simultaneously, separately, or sequentially. In some embodiments the deuterated [d6]-dextromethorphan hydrobromide is a deuterated [d6]-dextromethorphan hydrobromide hydrate, such as [d6]-dextromethorphan hydrobromide monohydrate.

The present disclosure provides, in some embodiments, a therapeutically effective amount of deuterated [d6]-dextromethorphan hydrobromide (d6-DM) for use in treating agitation associated with Alzheimer's disease in a patient having Alzheimer's disease, characterized in that the deuterated [d6]-dextromethorphan hydrobromide (d6-DM) is administered in combination with a therapeutically effective amount of quinidine sulfate (Q) wherein both medicaments are administered simultaneously, separately, or sequentially. In some embodiments the deuterated [d6]-dextromethorphan hydrobromide is a deuterated [d6]-dextromethorphan hydrobromide hydrate, such as [d6]-dextromethorphan hydrobromide monohydrate.

The present disclosure provides, in some embodiments, a combination of a therapeutically effective amount of deuterated [d6]-dextromethorphan hydrobromide (d6-DM) and a therapeutically effective amount of quinidine sulfate (Q) for use in treating agitation associated with Alzheimer's disease in a patient having Alzheimer's disease, wherein both medicaments are administered simultaneously, separately, or sequentially. In some embodiments the deuterated [d6]-dextromethorphan hydrobromide is a deuterated [d6]-dextromethorphan hydrobromide hydrate, such as [d6]-dextromethorphan hydrobromide monohydrate.

The present disclosure provides, in some embodiments, a pharmaceutical composition comprising a therapeutically effective amount of deuterated [d6]-dextromethorphan hydrobromide (d6-DM) which is used in combination with a therapeutically effective amount of quinidine sulfate (Q) simultaneously, separately, or sequentially, for treating agitation associated with Alzheimer's disease in a patient having Alzheimer's disease. In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, 36 mg d6-DM and 9.8 mg quinidine sulfate per day are provided in two doses, each dose containing 18 mg d6-DM and 4.9 mg quinidine sulfate. In some embodiments, the two doses are administered about 6, about 8, about 10, about 12, about 14, or about 16 hours apart. In some embodiments, the two doses are administered about 12 hours apart (e.g., morning and evening). In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments, 85.26 mg d6-DM and 9.8 mg quinidine sulfate per day are provided in two doses, each dose containing 42.63 mg d6-DM and 4.9 mg quinidine sulfate. In some embodiments, the two doses are administered about 6, about 8, about 10, about 12, about 14, or about 16 hours apart. In some embodiments, the two doses are administered about 12 hours apart (e.g., morning and evening). In some embodiments the deuterated [d6]-dextromethorphan hydrobromide is a deuterated [d6]-dextromethorphan hydrobromide hydrate, such as [d6]-dextromethorphan hydrobromide monohydrate.

In some embodiments of the methods herein, the method comprises
a) administering 36 mg d6-DM and 9.8 mg quinidine sulfate per day in two doses, each dose containing 18 mg d6-DM and 4.9 mg quinidine sulfate, for about 2 weeks; and
b) administering 56 mg d6-DM and 9.8 mg quinidine sulfate per day in two doses, each dose containing 28 mg d6-DM and 4.9 mg quinidine sulfate, for at least 1 week following the 2 week period in a); and
c) optionally administering 36 mg d6-DM and 9.8 mg quinidine sulfate per day in two doses, each dose containing 18 mg d6-DM and 4.9 mg quinidine sulfate, for at least 1 week following the 1 week period in b). In some embodiments the deuterated [d6]-dextromethorphan hydrobromide is a deuterated [d6]-dextromethorphan hydrobromide hydrate, such as [d6]-dextromethorphan hydrobromide monohydrate.

In some more particular embodiments, the at least 1 week in b) is at least two weeks.

In some more particular embodiments, the at least 1 week in b) is at least four weeks.

In some more particular embodiments, the at least 1 week in b) is at least six weeks.

In some more particular embodiments, the at least 1 week in b) is at least eight weeks.

In some more particular embodiments, the at least 1 week in b) is up to about nine weeks.

In some embodiments of the methods herein, the method comprises
a) administering 56 mg d6-DM and 9.8 mg quinidine sulfate per day in two doses, each dose containing 28 mg d6-DM and 4.9 mg quinidine sulfate, for about 2 weeks; and
b) administering 85.26 mg d6-DM and 9.8 mg quinidine sulfate per day in two doses, each dose containing 42.73 mg d6-DM and 4.9 mg quinidine sulfate, for at least 1 week following the 2 week period in a); and
c) optionally administering 56 mg d6-DM and 9.8 mg quinidine sulfate per day in two doses, each dose containing 28 mg d6-DM and 4.9 mg quinidine sulfate, for at least 1 week following the 1 week period in b).

In some more particular embodiments, the at least 1 week in b) is at least two weeks.

In some more particular embodiments, the at least 1 week in b) is at least four weeks.

In some more particular embodiments, the at least 1 week in b) is at least six weeks.

In some more particular embodiments, the at least 1 week in b) is at least eight weeks.

In some more particular embodiments, the at least 1 week in b) is up to about nine weeks.

As will be apparent to those skilled in the art, dosages outside of these disclosed dosages and ranges may be administered in some cases. Further, it is noted that the ordinary skilled clinician or treating physician will know how and when to interrupt, adjust, or terminate therapy in consideration of individual response.

In some embodiments provided herein is a method of treating agitation associated with Alzheimer's disease in a patient that has been diagnosed as having Alzheimer's disease, comprising:
administering to the patient therapeutically effective amounts of d6-DM and quinidine sulfate;
wherein the plasma concentration of d6-Dextromethorphan following the administering step is from about 20 μg/L to about 25 μg/L.

In some embodiments, the plasma concentration of d6-Dextromethorphan following the administering step is from 22.03 μg/L to 23.68 μg/L.

In some embodiments provided herein is a method of treating agitation associated with Alzheimer's disease in a patient that has been diagnosed as having Alzheimer's disease, comprising:
administering to the patient therapeutically effective amounts of d6-DM and quinidine sulfate;
wherein the plasma concentration of d6-Dextromethorphan following the administering step is from about 40 μg/L to about 50 μg/L.

In some embodiments, the plasma concentration of d6-Dextromethorphan following the administering step is from 43.80 μg/L to 49.21 μg/L.

In some embodiments provided herein is a method of treating agitation associated with Alzheimer's disease in a patient that has been diagnosed as having Alzheimer's disease, comprising:
administering to the patient therapeutically effective amounts of d6-DM and quinidine sulfate;
wherein the plasma concentration of d3-3-Methoxymorphinan following the administering step is from about 30 μg/L to about 40 μg/L.

In some embodiments, the plasma concentration of d6-Dextromethorphan following the administering step is from about 35 μg/L to about 40 μg/L, such as from 35.80 to 36.45 μg/L.

In some embodiments provided herein is a method of treating agitation associated with Alzheimer's disease in a patient that has been diagnosed as having Alzheimer's disease, comprising:
determining the CMAI total score in the patient;
administering to the patient therapeutically effective amounts of d6-DM and quinidine sulfate;
wherein the plasma concentration of d6-Dextromethorphan following the administering step is from about 20 μg/L to about 25 μg/L.

In some embodiments, the plasma concentration of d6-Dextromethorphan following the administering step is from 22.03 to 23.68 μg/L.

In some embodiments provided herein is a method of treating agitation associated with Alzheimer's disease in a patient that has been diagnosed as having Alzheimer's disease, comprising:
determining the CMAI total score in the patient; and
administering to the patient therapeutically effective amounts of d6-DM and quinidine sulfate;
wherein the plasma concentration of d6-Dextromethorphan following the administering step is from about 40 μg/L to about 50 μg/L.

In some embodiments, the plasma concentration of d6-Dextromethorphan following the administering step is from 43.80 μg/L to 49.21 μg/L.

In some embodiments provided herein is a method of treating agitation associated with Alzheimer's disease in a patient that has been diagnosed as having Alzheimer's disease, comprising:
determining the CMAI total score in the patient;
administering to the patient therapeutically effective amounts of d6-DM and quinidine sulfate;
wherein the plasma concentration of d3-3-Methoxymorphinan following the administering step is from about 30 μg/L to about 40 μg/L.

In some embodiments, the plasma concentration of d6-Dextromethorphan following the administering step is from about 35 μg/L to about 40 μg/L, such as from 35.80 to 36.45 μg/L.

In some embodiments provided herein is a method of treating agitation associated with Alzheimer's disease in a patient that has been diagnosed as having Alzheimer's disease, comprising:
1) determining the CMAI total score in the patient;
2) administering to the patient therapeutically effective amounts of d6-DM and quinidine sulfate;
wherein the plasma concentration of d6-Dextromethorphan following the administering step is from about 50 μg/L to about 70 μg/L.

In some embodiments, the plasma concentration of d6-Dextromethorphan following the administering step is from 54.82 to 64.41 μg/L.

In some embodiments provided herein is a method of treating agitation associated with Alzheimer's disease in a patient that has been diagnosed as having Alzheimer's disease, comprising:
1) administering to the patient therapeutically effective amounts of d6-DM and quinidine sulfate;
wherein the plasma concentration of d6-Dextromethorphan following the administering step is from about 125 μg/L to about 150 μg/L.

In some embodiments, the plasma concentration of d6-Dextromethorphan following the administering step is from about 130 to about 150 μg/L, such as from 131.07 to 145.49 μg/L.

In some embodiments provided herein is a method of treating agitation associated with Alzheimer's disease in a patient that has been diagnosed as having Alzheimer's disease, comprising:
1) administering to the patient therapeutically effective amounts of d6-DM and quinidine sulfate;
wherein the plasma concentration of d3-3-Methoxymorphinan following the administering step is from about 60 μg/L to about 95 μg/L.

In some embodiments, the plasma concentration of d6-Dextromethorphan following the administering step is from about 65 to about 90 μg/L, such as from 69.81 to 85.73 μg/L.

Oral administration can be employed for providing the patient with an effective dosage of d6-DM in combination with quinidine sulfate for agitation associated with Alzheimer's disease in a patient that has been diagnosed as having Alzheimer's disease. In some embodiments, the formulations can contain a combination of d6-DM and quinidine sulfate with pharmaceutically acceptable carriers or diluents known to those of skill in the art. In some embodiments, the d6-DM and the quinidine sulfate are administered orally. In some embodiments, the d6-DM and the quinidine sulfate are administered orally in a unit dosage form. In some embodiments, the unit dosage forms of the d6-DM and the quinidine sulfate are in the form of a capsule.

In some embodiments, a pharmaceutical composition comprising d6-DM and quinidine sulfate is in the form of a tablet. In some embodiments, a pharmaceutical composition comprising d6-DM and quinidine sulfate is in the form of a capsule.

The methods disclosed herein may also, optionally, include administration of the d6-DM and the quinidine sulfate in conjunction with other therapeutic agents, such as, for example, one or more therapeutic agents useful for the treatment of Alzheimer's disease.

Also provided herein are therapeutic uses of d6-DM and quinidine sulfate. An exemplary embodiment is the use of d6-DM and quinidine sulfate in treating agitation associated with Alzheimer's disease in a patient that has been diagnosed as having Alzheimer's disease. Another exemplary embodiment is the use of d6-DM and quinidine sulfate in a method of manufacturing a medicament for treating agitation associated with Alzheimer's disease in a patient that has been diagnosed as having Alzheimer's disease. Compositions useful for treating agitation associated with Alzheimer's disease are also provided.

In some embodiments of the methods disclosed herein, the patient is not being treated with certain additional therapeutic agents concomitantly with the d6-DM and the quinidine sulfate. In some embodiments, the patient has not taken certain additional therapeutic agent(s) within 2 weeks or 5 half-lives, whichever is longer, prior to the start of treatment with the d6-DM and the quinidine sulfate.

Another exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the patient is not being treated with one or more monoamine oxidase inhibitors (MAOIs). Exemplary MAOIs include but are not limited to carbamazepine, cyproterone, hyperforin, oxcarbazepine, phenobarbital, phenytoin, rifampicin, and St. John's Wort.

Another exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the patient is not being treated with clozapine.

Another exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the patient is not being treated with a typical antipsychotic. Exemplary typical antipsychotics include but are not limited to haloperidol, loxapine, thioridazine, molindone, thiothixene, fluphenazine, mesoridazine, trifluoperazine, perphenazine, and chlorpromazine.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient is not being treated with an agent that:
(a) increases levels of quinidine sulfate;
(b) is metabolized by CYP2D6;
(c) is related to quinidine sulfate;
(d) produces serotonin syndrome when co-administered with d6-DM;
(e) decreases plasma levels of d6-DM and quinidine sulfate;
(f) is clozapine,
(g) is a typical antipsychotic,
(h) is nefazodone;
(i) is a tricyclic antidepressant;
(j) is a monoamine oxidase inhibitors (MAOI);
(k) is a benzodiazepine,
(l) is a typical antipsychotic; or
(m) is selected from the group consisting of atomoxetine, carbamazepine, fosphenytoin, pentobarbital, phenobarbital, phenytoin, and primidone.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a Cohen-Mansfield agitation inventory (CMAI) total score of greater than or equal to 33, such as 33 to 203, such as 45 to 120, such as 55 to 90, prior to the administering step wherein the patient is not being treated with an agent that:
(a) increases levels of quinidine sulfate;
(b) is metabolized by CYP2D6;
(c) is related to quinidine sulfate;
(d) produces serotonin syndrome when co-administered with d6-DM;
(e) decreases plasma levels of d6-DM and quinidine sulfate;
(f) is clozapine,
(g) is a typical antipsychotic,
(h) is nefazodone;
(i) is a tricyclic antidepressant;
(j) is a monoamine oxidase inhibitors (MAOI);
(k) is a benzodiazepine,
(l) is a typical antipsychotic; or
(m) is selected from the group consisting of atomoxetine, carbamazepine, fosphenytoin, pentobarbital, phenobarbital, phenytoin, and primidone.

In some embodiments, the present disclosure provides a method of treating agitation associated with Alzheimer's disease in a patient that has been diagnosed as having Alzheimer's disease, comprising administering to the patient therapeutically effective amounts of d6-DM and quinidine sulfate, wherein the patient has been assessed as having a CMAI aggressive behavior score of greater than or equal to 15, such as 15 to 84, such as 15 to 70, such as 15 to 55, such as 15 to 40, such as 15, 16, 17, 18, 19 20, 21, 22, 23, 24, 25, 26, 27, 28, 29, 30, 31, 32, 33, 34, 35, 36, 37, 38, 39 or 40, wherein the patient is not being treated with an agent that:
(a) increases levels of quinidine sulfate;
(b) is metabolized by CYP2D6;
(c) is related to quinidine sulfate;
(d) produces serotonin syndrome when co-administered with d6-DM;
(e) decreases plasma levels of d6-DM and quinidine sulfate;
(f) is clozapine,
(g) is a typical antipsychotic,
(h) is nefazodone;
(i) is a tricyclic antidepressant;
(j) is a monoamine oxidase inhibitors (MAOI);
(k) is a benzodiazepine,
(l) is a typical antipsychotic; or
(m) is selected from the group consisting of atomoxetine, carbamazepine, fosphenytoin, pentobarbital, phenobarbital, phenytoin, and primidone.

In some embodiments, the patient is not being treated with an agent that increases levels of quinidine sulfate, compared to when the quinidine sulfate is administered without the agent. Exemplary agents that may increase levels of quinidine sulfate include but are not limited to amiodarone, a carbonic anhydrase inhibitor, cimetidine, diltiazem, itraconazole, ketoconazole, a macrolide antibiotic, a protease inhibitor, and voriconazole. Non-limiting examples of macrolide antibiotics include erythromycin, azithromycin, clarithromycin, dirithromycin, and roxithromycin. Non-limiting examples of protease inhibitors include saquinavir, ritonavir, atazanavir, and indinavir.

In some embodiments, the patient is not being treated with an agent that is metabolized by CYP2D6. Exemplary agents that are metabolized by CYP2D6 and may have increased plasma levels if co-administered with quinidine sulfate include but are not limited to dextromethorphan (over-the-counter or prescription), a tricyclic antidepressant (TCA), and atomoxetine. Non-limiting examples of TCAs include imipramine, desipramine, amitriptyline, and nortriptyline.

In some embodiments, the patient is not being treated with an agent that is related to quinidine sulfate. Exemplary agents that are related to quinidine sulfate include but are not limited to quinine and mefloquine.

In some embodiments, the patient is not being treated with an agent that may cause serotonin syndrome when co-administered with d6-DM. Exemplary agents that may cause serotonin syndrome when co-administered with d6-DM include but are not limited to MAOIs. Non-limiting examples of MAOIs include carbamazepine, cyproterone, hyperforin, oxcarbazepine, phenobarbital, phenytoin, rifampicin, and St. John's Wort.

Another exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the patient does not have a suicide risk. In some embodiments, suicide risk is determined by one or more of the following:
(a) judgment of the prescribing doctor;
(b) the patient answers yes on the Columbia Suicide Severity Rating Scale (C-SSRS Suicidal Ideation Item 4 (active suicidal ideation with some intent to act, without a specific plan) and the patient's most recent episode meeting this C-SSRS Item 4 occurred within six months;
(c) the patient answers yes on the C-SSRS Suicidal Behavior Item 5 (active suicidal ideation with specific plan and intent) and the patient's most recent episode meeting this C-SSRS Item 5 occurred within six months; or
(d) the patient answers yes on any of the 5 on the C-SSRS Suicidal Behavior Items (active attempt, interrupted attempt, aborted attempt, preparatory acts, or behavior) and the patient's most recent episode meeting any of these C-SSRS Items occurred within two years prior to treatment.

Another exemplary embodiment of the present disclosure includes a method of treating agitation associated with Alzheimer's disease in a patient that has been diagnosed as having Alzheimer's disease, comprising administering to the patient therapeutically effective amounts of d6-DM and quinidine sulfate, wherein the patient does not have a cardiovascular history of any one or more of.
(a) history or evidence of complete heart block, ventricular tachycardia, presence of clinically significant premature ventricular contractions (PVCs) as evaluated by a central reader, QTc prolongation, or torsades de pointes;
(b) QTc using the Fridericia's formula (QTcF) greater than 450 msec for males and greater than 470 msec for females based on central review, unless due to ventricular pacing;
(c) family history of congenital QT interval prolongation syndrome; or
(d) history or presence of clinically significant syncope, orthostatic hypotension, or postural tachycardia.

In some embodiments of the methods disclosed herein, the patient has been diagnosed as having Alzheimer's disease based on the Diagnostic and Statistical Manual of Mental Disorders (DSM) criteria for Alzheimer's disease. In some embodiments, the DSM criteria are the criteria set forth in the American Psychiatric Association's (2000) Diagnostic and Statistical Manual of Mental Disorders, 4^{th} Edition, Text Revision (DSM-IV-TR), which is incorporated herein by reference for the disclosure of such criteria. In some embodiments, the DSM criteria are the criteria set forth in the American Psychiatric Association's (2013) Diagnostic and Statistical Manual of Mental Disorders, 5^{th} Edition (DSM-V), which is incorporated herein by reference for the disclosure of such criteria.

In some embodiments, the patient's diagnosis of Alzheimer's disease based on the DSM criteria has been confirmed by the Mini International Neuropsychiatric Interview (M.I.N.I.). The M.I.N.I. is a brief structured diagnostic interview for psychiatric disorders, including those in DSM-IV and DSM-5. In some embodiments, the M.I.N.I. used to confirm the diagnosis of Alzheimer's disease is M.I.N.I. Version 6.0, based on the DSM-IV-TR criteria. In some embodiments, the M.I.N.I. used to confirm the diagnosis of agitation associated with Alzheimer's disease is M.I.N.I. Version 7.0.2, based on the DSM-V criteria.

In some embodiments of the methods disclosed herein, the patient has one, more than one, or all of the exemplary inclusion criteria described in any one of Examples 1-4 herein.

In some embodiments of the methods disclosed herein, the patient does not have one or more of the exemplary exclusion criteria described in Examples 1-4 herein.

In some embodiments of the methods disclosed herein, the patient is administered the d6-DM and the quinidine sulfate in conjunction with other therapeutic agents, such as, for example, one or more therapeutic agents known or identified for the treatment of Alzheimer's disease.

In some embodiments of a method disclosed herein, the patient has been treated or is being treated with an antidepressant other than nefazodone, a tricyclic antidepressant, or a monoamine oxidase inhibitors (MAOI) prior to the administering step.

In some embodiments of a method disclosed herein, the patient has been treated or is being treated with memantine prior to the administering step.

In some embodiments of a method disclosed herein, the patient has been treated or is being treated with an acetylcholinesterase inhibitor, such as donepezil, prior to the administering step.

In some embodiments of a method disclosed herein, the patient has been treated or is being treated with an atypical antipsychotic other than clozapine prior to the administering step. In some embodiments, the atypical antipsychotic is administered to the patient within the dose guidance from its U.S. package insert for the treatment of Alzheimer' disease. In some embodiments, the atypical antipsychotic is an oral and long-acting intramuscular injectable. In some embodiments, the atypical antipsychotic is a second-generation atypical antipsychotic drug (SGA). Exemplary SGAs include but are not limited to olanzapine, risperidone, paliperidone, quetiapine, aripiprazole, and lurasidone. In some embodiments, the patient the patient has been treated or is being treated prior to the administering step with a psychotropic medication that is also a CYP2D6 substrate. Examples of such medications include aripiprazole, risperidone, duloxetine, fluoxetine, fluvoxamine, mirtazapine, paroxetine, and venlafaxine. In some embodiments, the patient the patient has been treated or is being treated prior to the administering step with beta blocker medication that is also a CYP2D6 substrate. Examples of such medications include carvedilol, metoprolol, propranolol, and timolol.

In some embodiments, the patient is not being treated with more than one SGA. In some embodiments, the patient is not being treated with more than one SGA with the exception of low dose quetiapine (e.g., up to 50 mg at night) for insomnia.

d6-DM and quinidine sulfate may be formulated as active ingredients in one or more pharmaceutical compositions. Such pharmaceutical compositions may also contain a pharmaceutically acceptable carrier, and optionally, other therapeutic ingredients.

Pharmaceutical compositions can be prepared in forms such as powders, capsules, tablets, suspensions, sachets, cachets, solutions, and elixirs. Carriers such as starches, sugars, microcrystalline cellulose, diluents, granulating agents, lubricants, binders, disintegrating agents, and the like can be used in oral solid preparations. In some embodiments, the compositions are prepared as oral solid preparations (such as powders, capsules, and tablets). In some embodiments, the compositions are prepared as oral liquid preparations. In some embodiments, the oral solid preparations are capsules or tablets. If desired, capsules or tablets can be coated by standard aqueous or nonaqueous techniques.

Pharmaceutical compositions suitable for oral administration can be provided as discrete units such as capsules, cachets, sachets, patches, tablets, and aerosol sprays, each containing predetermined amounts of the active ingredients, as powder or granules, or as a solution or a suspension in an aqueous liquid, a non-aqueous liquid, an oil-in-water emulsion, or a water-in-oil liquid emulsion. Such compositions can be prepared by any of the conventional methods of pharmacy, but the majority of the methods typically include the step of bringing into association the active ingredients with a carrier that constitutes one or more ingredients. In general, the compositions are prepared by uniformly and intimately admixing the active ingredients with liquid carriers, finely divided solid carriers, or both, and then, optionally, shaping the product into the desired presentation.

For example, a tablet can be prepared by compression or molding, optionally, with one or more additional ingredients. Compressed tablets can be prepared by compressing in a suitable machine the active ingredient in a free-flowing form such as powder or granules, optionally mixed with a binder, lubricant, inert diluent, surface active, or dispersing agent. Molded tablets can be made by molding, in a suitable machine, a mixture of the powdered compound moistened with an inert liquid diluent.

In some embodiments, the d6-DM and the quinidine sulfate are administered together in the form of a capsule. In some embodiments, the capsule comprising the d6-DM and the quinidine sulfate is an immediate release capsule. In some embodiments, the capsule is a hard gelatin capsule. In some embodiments, the capsule is size 3.

In some embodiments, each capsule (or other composition comprising d6-DM and quinidine sulfate as active ingredients) also contains inactive ingredients. In some embodiments, the inactive ingredients may include croscarmellose sodium, microcrystalline cellulose, colloidal silicone dioxide, and/or magnesium stearate. In some embodiments, the inactive ingredients consist of or comprise croscarmellose sodium, microcrystalline cellulose, colloidal silicone dioxide, and magnesium stearate.

In some embodiments, any position in d6-DM designated as having D has a minimum deuterium incorporation of at least 80%, at least 85%, at least 87%, at least 90%, at least 95%, at least 97%, at least 99%, or at least 99.5%) at the designated position(s) in the d6-DM. Thus, in some embodiments, a composition comprising d6-DM can include a distribution of isotopologues of the compound, provided at least 80% of the isotopologues include a D at the designated position(s).

In some embodiments, any position in d6-DM designated as having D has a minimum deuterium incorporation of at least 90%, at least 95%, at least 97%, at least 99%, or at least 99.5%) at the designated position(s) in the d6-DM.

In some embodiments, d6-DM is substantially free of other isotopologues of the compound, e.g., less than 10%, less than 5%, less than 2%, less than 1%, or less than 0.5% of other isotopologues are present.

The synthesis of d6-DM can be readily achieved by synthetic chemists of ordinary skill. Relevant procedures and intermediates are disclosed, for instance in Kim et al. (Bioorg Med Chem Lett 2001, 11:1651) and Newman et al. (J Med Chem 1992, 35:4135).

A convenient method for synthesizing d6-DM according to some embodiments substitutes the appropriate deuterated intermediates and reagents in synthesis methods utilized for the preparation of dextromethorphan. These methods are described, for example, in U.S. Pat. No. 7,973,049.

### Quinidine

The present disclosure envisions the use of quinidine sulfate. Quinidine is a potent CYP2D6 inhibitor and has been particularly studied in this use (see, e.g., U.S. Pat. No. 5,206,248). The chemical structure of quinidine sulfate ((C₂₀H₂₄N₂O₂)₂.H₂SO₄.2H₂O) is as follows:

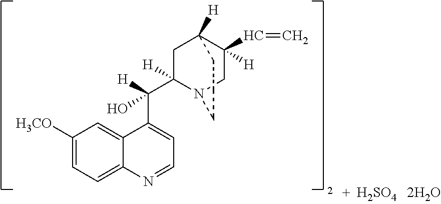

Quinidine administration can convert subjects with extensive metabolizer phenotype to poor metabolizer phenotype (Inaba et al. Br. J. Clin. Pharmacol. 1986; 22:199-200).

### Exemplary Scales

In some embodiments of the methods disclosed herein, one or more scales described herein, or others known in the art, may be used. Exemplary scales include but are not limited to the Cohen-Mansfield agitation inventory (CMAI) scale, the NPI scale, the MMSE scale, the Clinical Global Impression (CGI) Scales (e.g., Clinical Global Impression of Severity (CGI-S), Clinical Global Impression of Change (CGI-C), including the mADCS-CGIS scale and the mADCS-CGIC scale), the EQ-5D-5L scale, the RUD-Lite scale, and the S-STS scale.

The Cohen-Mansfield Agitation Inventory (CMAI) Scale

The CMAI Total Score

The CMAI total score is a score obtained by adding the scores for all agitated behaviors (“behaviors” are also referred to herein as “items”) in the Cohen-Mansfield Agitation Inventory (CMAI). The CMAI is a caregivers' rating questionnaire that identifies 29 agitated behaviors, each rated on a 7-point scale of frequency, where the ratings pertain to the two weeks preceding the administration of the CMAI. See INSTRUCTION MANUAL FOR THE COHEN-MANSFIELD AGITATION INVENTORY (CMAI), by J. Cohen-Mansfield (1991), incorporated by reference herein in its entirety and also referred to herein as the “CMAI Manual”.

The following are the 29 agitated behaviors:
1. Pacing and aimless wandering—constantly walking back and forth, including wandering when done in a wheelchair. Does not include normal purposeful walking.
2. Inappropriate dressing or disrobing—putting on too many clothes, putting on clothing in a strange matter (e.g., putting pants on head), taking off clothing in public or when it is inappropriate (if only genitals are exposed, rated under sexual advances). Does not include a person's ability to dress/undress as in ADL's.
3. Spitting (including while feeding)—spitting onto floor, other people, etc.; does not include uncontrollable salivating, or spitting into tissue, toilet, or onto ground outside.
4. Cursing or verbal aggression—only when using words; swearing, use of obscenity, profanity, unkind speech or criticism, verbal anger, verbal combativeness. Does not include unintelligible noises (rated under screaming or strange noises).
5. Constant unwarranted request for attention or help—verbal or nonverbal unreasonable nagging, pleasing, demoing (indicated also for oriented people).
6. Repetitive sentence or questions—repeating the same sentence or question one right after the other, addressed to a particular person or to no one (complaining, even if oriented and possibly warranted is rated under the complaining section).
7. Hitting (including self)—physical abuse, striking others, pinching others, banging self/furniture.
8. Kicking—striking forcefully with feet at people or objects.
9. Grabbing onto people or things inappropriately—snatching, seizing roughly, taking firmly, or yanking.
10. Pushing—forcefully thrusting, shoving, moving putting pressure against another.
11. Throwing things—hurling objects, violently tossing objects up in air, tipping off surfaces, flinging, dumping food.
12. Making strange noises—including crying, weeping, moaning, weird laughter, grinding teeth, does not include intelligible words.
13. Screaming—shoring, piercing howl, making loud shrills.
14. Biting—chopping, gnashing, gnawing, either other people or self.
15. Scratching—clawing, scarping with fingernails wither other people or self.
16. Trying to get to a different place—inappropriately entering or leaving a place, such as trying to get out of the building, off the property, sneaking out of a room, trying to get into locked areas, trespassing within unit, offices, or other resident's room or closet.
17. Intentional falling—purposefully falling onto floor, include from wheelchair, chair, or bed.
18. Complaining—whining, complaining about self, somatic complaints, personal gripes or complaining about physical environment or other people.
19. Negativism—bad attitude, doesn't like anything, nothing is right, does not include overt verbal anger, such as what can be rated as verbal aggression.
20. Eating or drinking inappropriate substances—putting into mouth and trying to swallow items that are inappropriate.
21. Hurting self or other—burning self or other, cutting self or other, touching self or other with harmful objects, etc.
22. Handling things inappropriately—picking up things that don't belong to them, rummaging through drawers, moving furniture, playing with good, fecal smearing.
23. Hiding things—putting objects out of sight, under or behind something.
24. Hoarding things—putting many or inappropriate objects in purse, pockets, or drawers, keeping too many of an item. (Does not include regular collections such as collecting dolls).
25. Tearing things or destroying property—shredding, ripping, breaking, stomping on something.
26. Performing repetitious mannerisms—stereotypic movement, such as patting, tapping, rocking self, fiddling with something, twiddling with something, rubbing self or object, sucking fingers, taking shoes on and off, picking at self, clothing, or objects, picking imaginary things out of air or off floor, manipulation of nearby objects in a repetitious manner, does not include repetitious words or vocalizations.
27. Making verbal sexual advances—sexual propositions, sexual innuendo, or “dirty” talk.
28. Making physical sexual advances or exposing genitals—touching a person in an inappropriate sexual way, rubbing genital area, inappropriate masturbation (when not alone in own room or bathroom), unwanted fondling or kissing.
29. General restlessness—fidgeting, always moving around in seat, getting up and sitting down inability to sit still.

The rating scale is as follows, based on the frequency of occurrence in the preceding two-week period.
1—Never
2—Less than once a week but still occurring
3—Once or twice a week
4—Several times a week
5—Once or twice a day
6—Several times a day
7—Several times an hour

The score for each CMAI behavior is obtained by rating how often the behavior was manifested by the individual being evaluated during the previous two-week period. The CMAI total score is obtained by adding the ratings for each of the 29 behaviors above. The 29 behaviors may be characterized into Factors or subscales as shown in Table 1A below, which is based on the factor analysis defined in Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998, also referred to herein as Rabinowitz et al. 2005, and incorporated herein in its entirety for all purposes. As Table 1A shows, 22 of the behaviors may be characterized as aggressive behaviors, physically non-aggressive behaviors, and verbally agitated behaviors, each of which is further discussed below. The remaining seven behaviors may be grouped separately (see “Other” in the diagram below):

TABLE 1A

| | Factor 2: Physically | | |
|---|---|---|---|
| Factor 1: Aggressive | Non-Aggressive | Factor 3: Verbally | |
| Behaviors | Behaviors | Agitated Behaviors | Other |
| Hitting | Pacing | Complaining | Making strange noise |
| Kicking | Inappropriate robing | Constant request for | Intentional falling |
| Pushing | or disrobing | attention | Eating or drinking |
| Scratching- | Trying to go to | Negativism | inappropriate |
| Tearing things | different place | Repetitious sentences | substances |
| Cursing or verbal | Handling things | or questions | Hiding |
| aggression | inappropriately | | Hoarding |
| Grabbing | General Restlessness | | Verbal sexual advances |
| Biting | Repetitions | | Physical sexual |
| Spitting | mannerism | | advances or exposing |
| Throwing things | | | |
| Screaming | | | |
| Hurt self or others | | | |

The CMAI Aggressive Behavior Score

As used herein, the CMAI aggressive behavior score is a score obtained by adding the ratings for aggressive behaviors (or “items”) in the Cohen-Mansfield Agitation Inventory (CMAI) as rated per the CMAI Manual based on behavior frequency as described elsewhere herein. The aggressive behaviors are based on the factor analysis in Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998. The aggressive behaviors are the following 12 items:
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; or
12) cursing or verbal aggression.

The following aggressive behavior items:
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; and
12) cursing or verbal aggression
   are also referred to as “CMAI Factor 1”, “Factor 1”, “CMAI F1”, “F1”, or “F1—Aggressive” behaviors.

The CMAI aggressive behavior score is obtained by adding the ratings for each of the 12 CMAI Factor 1 behaviors above. The CMAI aggressive behavior score is also referred to as the CMAI Factor 1 subscale score, CMAI F1—Aggressive Behavior score, or CMAI F1—Aggressive Behavior subscale score.

Based on the CMAI Manual, Factor 1 agitated status is defined as satisfying one of the following conditions:
a. ≥1 of the F1 behaviors occurring several times per week (score 4 or above), or
b. ≥2 of the F1 behaviors occurring once or twice per week (score 3 or above), or
c. ≥3 of the F1 behaviors occurring less than once per week (score 2 or above), or
d. 2 of the F1 behaviors occurring less than once per week (score 2 or above) and 1 of the F1 behaviors occurring once or twice per week (score 3 or above).

The lack of all the preceding conditions for Factor 1 agitated status indicates a Factor 1 not agitated status.

The CMAI Physically Nonaggressive Behavior Score

As used herein, the CMAI physically nonaggressive behavior score is a score obtained by adding the ratings for all physically nonaggressive behaviors (or “items”) in the Cohen-Mansfield Agitation Inventory (CMAJ) as rated per the CMAI Manual based on behavior frequency as described elsewhere herein. The physically nonaggressive behaviors are based on the factor analysis in Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998. The physically nonaggressive behaviors are the following 6 items:
1) pacing and/or aimless wandering
2) trying to get to a different place;
3) general restlessness;
4) inappropriate dressing or disrobing;
5) handling things inappropriately; or
6) performing repetitious mannerisms.

The CMAI physically nonaggressive behavior score is obtained by adding the ratings for each of the 6 behaviors above. The CMAI physically nonaggressive behavior score is also referred to as the CMAI Factor 2 subscale score, CMAI F2—Physically Non-Aggressive Behavior score, or CMAI F2—Physically Non-Aggressive Behavior subscale score. The 6 physically nonaggressive behaviors above are also referred to as “CMAI Factor 2”, “Factor 2”, “CMAI F2”, “F2”, or “F2—Physically Non-Aggressive” behaviors.

Based on the CMAI Manual, Factor 2 agitated status is defined as satisfying one of the following conditions:

a. ≥1 of the F2 behaviors occurring once or twice per day (score 5 or above), or
b. ≥2 of the F2 behaviors occurring several times per week (score 4 or above), or
c. ≥3 of the F2 behaviors occurring once or twice per week (score 3 or above), or
d. ≥4 of the F2 behaviors occurring less than once per week (score 2 or above).

The lack of all the preceding conditions for Factor 2 agitated status indicates a Factor 2 not agitated status.

The CMAI Verbally Agitated Behavior Score

As used herein, the CMAI verbally agitated behavior score is a score obtained by adding the ratings for all verbally agitated behaviors (or “items”) in the Cohen-Mansfield Agitation Inventory (CMAI) as rated per the CMAI Manual based on behavior frequency as described elsewhere herein. The verbally agitated behaviors are based on the factor analysis in Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998. The verbally agitated behaviors are the following 4 items:
1) complaining;
2) constant unwarranted requests for attention and/or help;
3) repetitive sentences or questions; or
4) negativism.

The CMAI verbally agitated behavior score is obtained by adding the ratings for each of the 4 behaviors above. The CMAI verbally agitated behavior score is also referred to as the CMAI Factor 3 subscale score, CMAI F3—Verbally Agitated Behavior score, or CMAI F3—Verbally Agitated Behavior subscale score. The 4 verbally agitated behaviors above are also referred to as “CMAI Factor 3”, “Factor 3”, “CMAI F3”, “F3”, or “F3—Verbally Agitated” behaviors.

Based on the CMAI Manual, Factor 3 agitated status is defined as satisfying one of the following conditions:

a. ≥1 of the F3 behaviors occurring once or twice per day (score 5 or above), or
b. ≥2 of the F3 behaviors occurring several times per week (score 4 or above), or
c. ≥3 of the F3 behaviors occurring once or twice per week (score 3 or above), or
d. ≥4 of the F3 behaviors occurring less than once per week (score 2 or above).

The lack of all the preceding conditions for Factor 3 agitated status indicates a Factor 3 not agitated status.

The CMAI Scale Agitated Status

CMAI Agitated Status is defined as the presence of any one CMAI subscale factor (F1—Aggressive Behavior, F2—Physically Nonaggressive Behavior, or F3—Verbally Agitated Behavior) scored as having agitated status.

### The NPI Scale

The NPI-AA Score

As used herein, the NPI-AA aggressive behavior score is a score obtained from the following questions in the Neuropsychiatric Inventory (NPI). See also NEUROPSYCHIATRIC INVENTORY (NPI): Instructions for Use and Administration, incorporated by reference herein in its entirety, and appended hereto as Appendix B:

Does the patient have periods when he/she refuses to cooperate or won't let people help him or her? Is he/she hard to handle?
If the answer, is NO, proceed to the next screening question.
If the answer, is YES, proceed to subquestions.
   i) Does the patient get upset with those trying to care for him/her or resist activities such as bathing or changing clothes?
   ii) Is the patient stubborn, having to have things his/her way?
   iii) Is the patient uncooperative, resistive to help from others?
   iv) Does the patient have any other behaviors that make him hard to handle?
   v) Does the patient shout or curse angrily?
   vi) Does the patient slam doors, kick furniture, throw things?
   vii) Does the patient attempt to hurt or hit others?
   viii) Does the patient have any other aggressive or agitated behaviors?

If the screening question is confirmed, determine the frequency and severity of the agitation:

Frequency:
1. Occasionally—less than once per week.
2. Often—about once per week.
3. Frequently—several times per week but less than daily.
4. Very frequently—once or more per day.

Severity:
1. Mild—behavior is disruptive but can be managed with redirection or reassurance.
2. Moderate—behaviors are disruptive and difficult to redirect or control
3. Marked—agitation is very disruptive and a major source of difficulty; there may be a threat of personal harm. Medications are often required.

Total:

Total=Frequency×Severity

Distress: How emotionally distressing do you (the caregiver) find this behavior?
0. Not at all
1. Minimally
2. Mildly
3. Moderately
4. Severely
5. Very severely or extremely

The NPI-AA score can be calculated by adding the scores Total score and the Distress score.

The NPI Aberrant Motor Behavior Domain Score

As used herein, the NPI Aberrant Motor Behavior domain score is a score obtained from the following questions in the Neuropsychiatric Inventory (NPI). See also NEUROPSYCHIATRIC INVENTORY (NPI): Instructions for Use and Administration, incorporated by reference herein in its entirety:

Does the patient pace, do things over and over such as opening closets or drawers, or repeatedly pick at things or wind strings or threads?

If the answer, is NO, proceed to the next screening question.

If the answer, is YES, proceed to subquestions.
i) Does the patient pace around the house without apparent purpose?
ii) Does the patient rummage around opening and unpacking drawers or closets?
iii) Does the patient repeatedly put on and take off clothing?
iv) Does the patient have repetitive activities or “habits” that he/she performs over and over?
v) Does the patient engage in repetitive activities such as handling buttons, picking, wrapping string, etc.?
vi) Does the patient fidget excessively, seem unable to sit still, or bounce his/her feet or tap his/her fingers a lot?
vii) Does the patient do any other activities over and over?

If the screening question is confirmed, determine the frequency and severity of the agitation:

Frequency:
1. Occasionally—less than once per week.
2. Often—about once per week.
3. Frequently—several times per week but less than every day.
4. Very frequently—essentially continuously present.

Severity:
1. Mild—abnormal motor activity is notable but produces little interference with daily routines.
2. Moderate—abnormal motor activity is very evident; can be overcome by the caregiver.
3. Marked—abnormal motor activity is very evident, usually fails to respond to any intervention by the caregiver, and is a major source of distress

Total:

Total=Frequency×Severity

Distress: How emotionally distressing do you (the caregiver) find this behavior?
0. Not at all
1. Minimally
2. Mildly
3. Moderately
4. Severely
5. Very severely or extremely

The NPI-AA Aberrant Motor Behavior domain score can be calculated by adding the Total score and the Distress score.

The NPI Irritability/Lability Domain Score

As used herein, the NPI Irritability/Lability domain score is a score obtained from the following questions in the Neuropsychiatric Inventory (NPI). See also NEUROPSYCHIATRIC INVENTORY (NPI): Instructions for Use and Administration, incorporated by reference herein in its entirety:

Does the patient get irritated and easily disturbed? Are his/her moods very changeable?We do not mean frustration over memory loss or inability to perform usual tasks; we are interested to know if the patient has abnormal irritability, impatience, or rapid emotional changes different from his/her usual self.

If the answer, is NO, proceed to the next screening question.

If the answer, is YES, proceed to subquestions.
i) Does the patient have a bad temper, flying “off the handle” easily over little things?
ii) Does the patient rapidly change moods from one to another, being fine one minute and angry the next?
iii) Does the patient have sudden flashes of anger?
iv) Is the patient impatient, having trouble coping with delays or waiting for planned activities?
v) Is the patient cranky and irritable?
vi) Is the patient argumentative and difficult to get along with?
vii) Does the patient show any other signs of irritability?

If the screening question is confirmed, determine the frequency and severity of the agitation:

Frequency:
1. Occasionally—less than once per week.
2. Often—about once per week.
3. Frequently—several times per week but less than every day.
4. Very frequently—essentially continuously present.

Severity:
4. Mild—irritability or lability is notable but usually responds to redirection and reassurance.
5. Moderate—irritability and lability are very evident and difficult to overcome by the caregiver.
6. Marked—irritability and lability are very evident, they usually fail to respond to any intervention by the caregiver, and they are a major source of distress

Total:

Total=Frequency×Severity

Distress: How emotionally distressing do you (the caregiver) find this behavior?
0. Not at all
1. Minimally
2. Mildly
3. Moderately
4. Severely
5. Very severely or extremely

The NPI-AA Irritability/Lability domain score can be calculated by adding the Total score and the Distress score.

### The MMSE Scale

The MMSE is a 30-point questionnaire test that is used to screen for cognitive impairment. It is commonly used in medicine to screen for dementia. The MMSE scale comprises 11 questions or simple tasks concerning orientation, memory, attention, and language to evaluate the patient's cognitive state. The MMSE total score ranges from 0 to 30, with higher scores indicating better cognitive function.

### Clinical Global Impression (CGI) Scales

The CGI was developed to provide a brief, stand-alone assessment of the clinician's view of the patient's global functioning prior to and after initiating a treatment (Busner and Targum, Psychiatry (Edgmont). 2007; 4(7):28-37). The CGI provides an overall clinician-determined summary measure that takes into account all available information, including knowledge of the patient's history, psychosocial circumstances, symptoms, behavior, and the impact of the symptoms on the patient's ability to function. The CGI comprises 2 companion 1-item measures, the CGI-S(Severity) and CGI-C(Change).

### Clinical Global Impression-Severity (CGI-S)

The CGI-S is a 7-point scale that requires the clinician to rate the severity of the patient's illness at the time of assessment, relative to the clinician's past experience with patients who have the same diagnosis (Guy, ECDEU Assessment Manual for Psychopharmacology. 1976:76-338). Considering total clinical experience, a patient is assessed on severity of mental illness at the time of rating 1, normal, not at all ill; 2, borderline mentally ill; 3, mildly ill; 4, moderately ill; 5, markedly ill; 6, severely ill; or 7, among the most extremely ill patients.

### Clinical Global Impression-Change (CGI-C)

The CGI-C is a 7-point scale that requires the clinician to rate the change of the patient's condition at the time of assessment, relative to the clinician's past experience with the patient's condition at admission. Considering total clinical experience, a patient is assessed for change of mental illness as 1, Very much improved; 2, Much improved; 3, Minimally improved; 4, No change; 5, Minimally worse; 6, Much worse; or 7, Very much worse.

In some embodiments, agitation associated with Alzheimer's disease is evaluated using the CGI (e.g., the CGI-S and/or CGI-C). In some embodiments, the CGI (e.g., the CGI-S and/or CGI-C) is used alone. In some embodiments, the CGI (e.g., the CGI-S and/or CGI-C) is used in combination with one or more additional scales (e.g., any one or more of the exemplary scales described herein).

### The CGIS-Agitation Scale

The CGIS-Agitation is a 7-point (1-7) scale (1=normal, not at all ill; 7=among the most extremely ill patients) and assesses severity of agitation in this study. The CGIS-Agitation is assessed at Screening (Day −28 to Day −1), Baseline (Day 1), Visit 2 (Day 8), Visit 3 (Day 15), Visit 4 (Day 29), Visit 5 (Day 43), Visit 6 (Day 57), Visit 7 (Day 71), Visit 8 (Day 85/ET), and the Follow-up visit (30 days postdose). The CGIS-Agitation must be administered by the same rater at each visit.

### Clinical Study Design

In the clinical study in Example 1 below, the benefit of treating agitation associated with Alzheimer's disease by administering d6-DM and quinidine sulfate was assessed.

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1

A Phase 3, multicenter study to assess the efficacy, safety, and tolerability of AVP-786 (deudextromethorphan hydrobromide [d6-DM]/quinidine sulfate [Q]) for the treatment of agitation in patients with dementia of the Alzheimer's type

Synopsis

Investigational Product:

AVP-786 (deudextromethorphan hydrobromide [d6-DM]/quinidine sulfate [Q])

Name of Active Ingredient(s):

deudextromethorphan hydrobromide [d6-DM] and quinidine sulfate [Q]

Title of Study:

A Phase 3, multicenter study to assess the efficacy, safety, and tolerability of AVP-786 (deudextromethorphan hydrobromide [d6-DM]/quinidine sulfate [Q]) for the treatment of agitation in patients with dementia of the Alzheimer's type

Objectives:

The primary objective is to:
Evaluate the efficacy, safety, and tolerability of AVP-786 for the treatment of agitation in patients with dementia of the Alzheimer's type

The secondary objectives are to:
Evaluate the effects of AVP-786 on global assessments of severity and improvement of agitation
Evaluate the effects of AVP-786 on neuropsychiatric symptoms
Evaluate the effects of AVP-786 on measures of quality of life and resource utilization

Study Design: Phase 3, multicenter study

Methodology:

Screening Period (Days −28 to −1): A protocol eligibility form is completed for each patient and reviewed by a Medical Monitor for approval prior to participation in the study.

12-week Treatment Period (Days 1-85)

30-day Follow-up Period. All enrolled patients, whether they complete the study or terminate from the study early for any reason, have a Follow-up visit 30 days after the last dose of study drug for select efficacy and safety assessments.

Assessments and Visits: Patients attend clinic visits at Screening, Baseline (Day 1), Visit 2 (Day 8), Visit 3 (Day 15), Visit 4 (Day 29), Visit 5 (Day 43), Visit 6 (Day 57), Visit 7 (Day 71), Visit 8/Early Termination (ET) (Day 85), and Follow-up (30 days after the last dose).

Study procedures performed at each visit are outlined in the Schedule of Assessments (Table 1).

Diagnosis and Main Criteria for Inclusion: Patients with agitation secondary to Alzheimer's dementia; the diagnosis of probable Alzheimer's disease is based on the ‘2011 Diagnostic Guidelines for Alzheimer's Disease’ issued by the National Institute on Aging (NIA)-Alzheimer's Association (AA) workgroups. Diagnosis of agitation is based on the provisional consensus definition of agitation in patients with cognitive disorders developed by the International Psychogeriatric Association (IPA) Agitation Definition Work Group.

Key Inclusion Criteria: Patients 50 to 90 years of age (inclusive) with clinically significant, moderate-to-severe agitation for at least 2 weeks prior to Screening that interferes with daily routine per the Investigator's judgment, and who require pharmacotherapy for the treatment of agitation per the Investigator's judgment after an evaluation of reversible factors and a course of nonpharmacological interventions. A Neuropsychiatric Inventory Agitation/Aggression (NPI-AA) score of ≥4 and Mini Mental State Examination (MMSE) score of 8 to 24 (inclusive) at Screening and Baseline are required for study participation.

Key Exclusion Criteria: Patients with dementia predominantly of the non-Alzheimer's type (e.g., vascular dementia, frontotemporal dementia, Parkinson's disease, substance-induced dementia) and patients with symptoms of agitation that are not secondary to Alzheimer's dementia (e.g., secondary to pain, other psychiatric disorder, or delirium) are not eligible.

Investigational Product, Dosage and Mode of Administration:

AVP-786 capsule is administered orally BID at a dose of AVP-786-18 (d6-DM 18 mg/Q 4.9 mg) or AVP-786-42.63 (d6-DM 42.63 mg/Q 4.9 mg).

Duration of Treatment: Patients are enrolled in the study for approximately 20 weeks, which includes:
Up to a 28-day Screening Period
12-week Double-blind Treatment Period
30-day Follow-up Period

Criteria for Evaluation:

Efficacy:

Primary Efficacy Measure: Cohen-Mansfield Agitation Inventory (CMAI)

Key Secondary Efficacy Measure: Clinical Global Impression of Severity of Illness for Agitation (CGIS-Agitation)

Other Efficacy Measures: Other efficacy measures include, Clinical Global Impression of Change (CGIC-Agitation), NPI-AA, NPI total, EuroQol 5-Dimension 5-Level (EQ-5D-5L), and Resource Utilization in Dementia-Lite (RUD-Lite).

Pharmacokinetics: Plasma concentrations of d6-DM, its metabolites d3-dextrorphan (d3-DX) and d3-3-methoxymorphinan (d3-3-MM), and Q are measured. Urine concentrations of d6-DM and its metabolite d3-DX are measured.

Safety: Safety and tolerability of AVP-786 is assessed by reported adverse events (AEs), physical and neurological examinations, vital signs, clinical laboratory assessments, resting 12-lead electrocardiograms (ECGs), MMSE, the Epworth Sleepiness Scale (ESS), and the Sheehan Suicidality Tracking Scale (S-STS).

Efficacy Analyses: The primary efficacy endpoint is the change from Baseline to the end of the efficacy period in the CMAI total score.

All efficacy analyses are based on the intent-to-treat analysis set, defined as all patients in the randomized population who take at least 1 dose of study drug (AVP-786), have a Baseline, and at least 1 post-Baseline evaluation for the CMAI total score. Descriptive statistics are provided for all efficacy variables in general. Continuous variables are summarized by tabulations of mean, median, range, and standard deviation (SD). Tabulations of frequency distributions are provided for categorical variables. The primary endpoint is analyzed using mixed-effect model repeat measures (MMRM).

Pharmacokinetic Analyses: Plasma concentrations of d6-DM, its metabolites d3-DX and d3-3-MM, and Q are summarized descriptively. Urine concentrations of d6-DM and its metabolite d3-DX are summarized descriptively.

Safety Analyses: Safety analyses are based on safety population defined as all patients who are randomized and take at least one dose of study drug. It consists of data summaries for biological parameters and AEs. Descriptive statistics are provided for all safety variables in general. Continuous variables are summarized by tabulations of mean, median, range, and SD. Tabulations of frequency distributions are provided for categorical variables. Safety analyses are tabulated by treatment. AEs are coded using the Medical Dictionary for Regulatory Activities (MedDRA). Summary statistics of absolute values and percentage change from baseline for blood pressure (systolic and diastolic), heart rate, respiratory rate, and ECG parameters are provided. Laboratory parameters are summarized via descriptive statistics and via shifts in results with respect to normal ranges between Baseline and end of treatment as increased, decreased, or no change. The S-STS, MMSE, and ESS are summarized via descriptive statistics.

TABLE 1
Schedule of Assessments and Visits

| | 12-WEEK DOUBLE-BLIND TREATMENT PERIOD | | | | | | | | |
|---|---|---|---|---|---|---|---|---|---|
| Visit: | | | | | | | | | |
| | | Visit | Visit | Visit | Visit | Visit | Visit | Visit 8 | Follow-up |
| Screening^{a} | Baseline | 2 | 3 | 4 | 5 | 6 | 7 | (or ET) | Visit ^{b} |
| Study Day: | | | | | | | | | |
| | | | | | | | | | 30 (+7) days |
| Day −28 | | Day 8 | Day 15 | Day 29 | Day 43 | Day 57 | Day 71 | Day 85 | Post Last |
| to −1 | Day 1 | (±3 days) | (±3 days) | (±3 days) | (±3 days) | (±3 days) | (±3 days) | (±3 days) | Dose |

End of Study Week:

| | | | | | | | |
|---|---|---|---|---|---|---|---|
| Procedure | Week 1 | Week 2 | Week 4 | Week 6 | Week 8 | Week 10 | Week 12 |

ELIGIBILITY and HISTORY

| | | |
|---|---|---|
| Signed informed consent | X | |
| forms | | |
| Inclusion and exclusion | X | X |
| criteria | | |
| Medical, psychiatric, and | X | |
| neurological history | | |
| Risk assessment for falls | X | |
| (worksheet and TUG) | | |
| Hachinski Ischemic Scale | X | |
| (Rosen Modification) | | |
| Protocol eligibility form^{c} | X | |

EFFICACY

| | | | | | | | | | | |
|---|---|---|---|---|---|---|---|---|---|---|
| CMAI | X | X | X | X | X | X | X | X | X | X |
| CGIS-Agitation | X | X | X | X | X | X | X | X | X | X |
| CGIC-Agitation | | | X | X | X | X | X | X | X | |
| NPI^{d} | X^{d} | X | X | X^{d} | X^{d} | X | X^{d} | X | X | |
| EQ-5D-5L | | X | | | | | | | X | |
| RUD-Lite | | X | | | | | | | X | |

SAFETY

| | | | | | | | | | | |
|---|---|---|---|---|---|---|---|---|---|---|
| Vitals signs | X | X | X | X | X | X | X | X | X | |
| Weight and height^{e} | | X^{e} | | | | | | | X^{e} | |
| Physical and neurological | X | | | | | | | | X | |
| examination | | | | | | | | | | |
| ECG | X^{f} | X^{g} | X^{g} | | | X^{h} | | | X^{h} | |
| Chemistry, hematology, | Xⁱ | | | | | X | | | Xⁱ | |
| urinalysis | | | | | | | | | | |
| Urine pregnancy test^{j} | X | X | | | | | | | X | |
| Adverse events | X | X | X | X | X | X | X | X | X | X |
| Prior and concomitant | X | X | X | X | X | X | X | X | X | X |
| medications, non-drug | | | | | | | | | | |
| therapies, | | | | | | | | | | |
| nonpharmacological | | | | | | | | | | |
| interventions for agitation | | | | | | | | | | |
| MMSE | X | X | | | | | | | X | |
| ESS^{k} | | X | | | | | | | X | |
| S-STS | X | X | | | | | | | X | |

OTHER PROCEDURES

| | | | | | | | | |
|---|---|---|---|---|---|---|---|---|
| PK blood sample^{l} | | X | | | X | | X | |
| PK urine sample^{m} | | X | | | | | | |
| CYP2D6 blood sample | | X | | | | | | |
| Amyloid β blood sample | | X | | | | | | |
| Administer morning dose of | X | X | | | X | | | |
| study drug in clinic | | | | | | | | |
| Dispense blister card and | X | X | X | X | X | X | X | |
| diary cards | | | | | | | | |
| Review blister card and | | X | X | X | X | X | X | X |
| diary cards | | | | | | | | |

AE = adverse event;
CGIC-Agitation = Clinical Global Impression of Change for Agitation;
CGIS-Agitation = Clinical Global Impression of Severity of Illness for Agitation;
CMAI = Cohen-Mansfield Agitation Inventory;
ECG = electrocardiogram;
EQ-5D-5L = EuroQol 5-Dimension 5-Level;
ESS = Epworth Sleepiness Scale;
ET = early termination;
MMSE = Mini Mental State Examination;
NPI = Neuropsychiatric Inventory;
PK = Pharmacokinetics;
RUD-Lite = Resource Utilization in Dementia-Lite;
S-STS = Sheehan Suicidality Tracking Scale;
TUG = Timed Up and Go
^{a}The Screening period may be extended after discussion with and approval by a Medical Monitor.
^{b} All enrolled patients have an in-clinic Follow-up visit 30 (+7) days after last dose of study drug for selected safety and efficacy assessments.
^{c}For each patient, a protocol eligibility form is completed by the site and reviewed by a Medical Monitor for approval prior to participation in the study.
^{d}Only the Agitation/Aggression domain of the NPI is performed at Screening, and at Visit 3, Visit 4, and Visit 6 (i.e., Days 15, 29, and 57).
^{e}Height and weight are measured at Baseline (Day 1); only weight is measured at Visit 8 (Day 85/ET).
^{f}At Screening, 3 ECGs are performed (e.g., one after the other).
^{g}ECG is performed predose and 1 to 1.5 hours postdose at Baseline (Day 1) and Visit 2 (Day 8).
^{h}ECG is performed at any time at Visit 5 (Day 43) and Visit 8 (Day 85/ET).
ⁱThyroid function tests (TSH, and reflex T3 and T4 if TSH is abnormal) are performed at Screening. Glycosylated hemoglobin (HbA1c) test is performed at Screening and Visit 8 (Day 85/ET).
^{j}Urine pregnancy test is performed for women of child-bearing potential only.
^{k}ESS is rated only by patients who have a MMSE score of ≥10 at Baseline.
^{l}At Visit 2 (Day 8), the PK blood sample is collected 1 to 4 hours postdose. At Visit 5 (Day 43), the PK blood sample is collected predose. At Visit 7 (Day 71), the PK blood sample is collected at any time. The time of the last 2 doses of study drug prior to collection of the PK blood sample is recorded in the clinical database.
^{m}At Visit 2 (Day 8), the PK urine sample is collected 1 to 4 hours postdose.

### List of Abbreviations and Definitions of Terms

The following abbreviations and specialist terms are used in this Example 1.

TABLE 2
Abbreviations and Specialist Terms

| Abbreviation | Definition |
|---|---|
| ADWG | Agitation Definition Working Group |
| ANCOVA | analysis of covariance |
| AVP-786-18 | AVP-786 containing d6-DM 18 mg/ |
| | Q 4.9 mg |
| AVP-786-42.63 | AVP-786 containing d6-DM 42.63 mg/ |
| | Q 4.9 mg |
| BP | blood pressure |
| CFR | US Code of Federal Regulations |
| CGIC-Agitation | Clinical Global Impression of Change |
| CGIS | Clinical Global Impression of Severity |
| CGIS-Agitation | Clinical Global Impression of Severity |
| | of Illness scale for Agitation |
| CMAI | Cohen-Mansfield Agitation Inventory |
| CMH | Cochran-Mantel-Haenszel |
| CNS | central nervous system |
| CYP | cytochrome P450 |
| d6-DM | deudextromethorphan hydrobromide |
| | (or deudextromethorphan) |
| DEMQOL | Dementia Quality of Life |
| DMP | data management plan |
| DSMB | Data Safety Monitoring Board |
| DSM-V-TR | Diagnostic and Statistical Manual of |
| | Mental Disorders, 5th Edition, Text |
| | Revision |
| eCRF | electronic case report form |
| ECG | electrocardiogram |
| EDC | electronic data capture |
| EP | European Pharmacopoeia |
| EQ-5D-5L | EuroQol 5-Dimension 5-Level |
| ESS | Epworth Sleepiness Scale |
| FDA | Food and Drug Administration |
| GCP | Good Clinical Practice |
| Abbreviation | Definition |
| GMP | Good Manufacturing Practice |
| HbA1c | glycosylated hemoglobin |
| HR | heart rate |
| IAP | interim analysis plan |
| ICF | informed consent form |
| ICH | International Council for Harmonisation |
| IEC/EC | Independent Ethics Committee |
| ITT | intent-to-treat |
| IRB | Institutional Review Board |
| IWRS | interactive web-response system |
| LOCF | last observation carried forward |
| mADCS-CGIC-Agitation | Modified Alzheimer’s Disease |
| | Cooperative Study-Clinical Global |
| | Impression of Change scale for |
| | Agitation |
| MAOI | monoamine oxidase inhibitor |
| MedDRA | Medical Dictionary for Regulatory |
| | Activities |
| mITT | modified intent-to-treat |
| MMRM | mixed-effect model repeated measures |
| MMSE | Mini Mental State Examination |
| MNAR | missing data being missing not at random |
| NIA-AA | National Institute on Aging-Alzheimer’s |
| | Association |
| NPI | Neuropsychiatric Inventory |
| NPI-AA | Neuropsychiatric Inventory Agitation/ |
| | Aggression |
| NPI-NH | Neuropsychiatric Inventory-Nursing |
| | Home |
| OC | observed case |
| OTC | over-the-counter |
| PK | pharmacokinetic |
| PVC | premature ventricular contractions |
| Q | quinidine sulfate (or quinidine) |
| QOL | quality of life |
| QTcF | QTc by Fridericia's formula |
| RUD-Lite | Resource Utilization in Dementia-Lite |
| SAE | serious adverse event |
| SAP | statistical analysis plan |
| SOC | system organ class |
| SNRI | serotonin-norepinephrine reuptake |
| | inhibitor |
| SSRI | selective serotonin reuptake inhibitors |
| S-STS | Sheehan Suicidality Tracking Scale |
| TEAE | treatment-emergent adverse event |
| TSH | thyroid-stimulating hormone |
| TUG | Timed Up and Go |
| UN | unstructured variance covariance |
| | structure |
| US | United States |
| USP | United States Pharmacopoeia |

### 1. Investigational Plan

### 1.1. Overall Study Design

This is a Phase 3, multicenter study with a 12-week treatment duration. The study consists of a 4-week Screening period, a 12-week double-blind treatment period, and a 30-day Follow-up period.

Screening Period (Day −28 to Day −1)

Patient eligibility is determined during the Screening visit, which occurs within 4 weeks of the Baseline visit. A protocol eligibility form is completed for each patient and reviewed by a Medical Monitor for approval prior to participation in the study.

Treatment Period (12 Weeks)

Study drug is administered twice daily (BID; morning and evening) starting from the Baseline visit (Day 1) through Visit 8 (Day 85).

Follow-Up Period

All enrolled patients, whether they complete the study or terminate from the study early for any reason, have a Follow-up visit 30 days after the last dose of study drug for select efficacy and safety assessments.

Assessments and Visits:

Patients attend clinic visits at Screening (Day −28 to −1), Baseline (Day 1), Visit 2 (Day 8), Visit 3 (Day 15), Visit 4 (Day 29), Visit 5 (Day 43), Visit 6 (Day 57), Visit 7 (Day 71), Visit 8 (Day 85; or Early Termination [ET] Visit), and 30 days after the last dose of study drug (Follow-up visit).

Study assessments and procedures are performed at each visit as outlined in the Schedule of Assessments and Visits ((Table 1). The primary efficacy measure is the Cohen-Mansfield Agitation Inventory (CMAI). Secondary efficacy measures include Clinical Global Impression of Severity of Illness for Agitation (CGIS-Agitation), Clinical Global Impression of Change for Agitation (CGIC-Agitation), Neuropsychiatric Inventory Agitation/Aggression (NPI-AA), NPI total, EuroQol 5-Dimension 5-Level (EQ-5D-5L), and Resource Utilization in Dementia-Lite (RUD-Lite).

Pharmacokinetic (PK) measurements of plasma concentrations of d6-DM, its metabolites d3-DX and d3-3-MM, and Q are measured from blood samples collected at Visit 2 (Day 8), Visit 5 (Day 43), and Visit 7 (Day 71). PK measurements of urine concentrations of d6-DM and its metabolite d3-DX are measured from a urine sample collected at Visit 2 (Day 8).

The safety and tolerability of AVP-786 are assessed by reported AEs, physical and neurological examination, vital signs, clinical laboratory measures, resting 12-lead electrocardiograms (ECG), and the following safety scales: Mini-Mental State Examination (MMSE), Epworth Sleepiness Scale (ESS), and Sheehan Suicidality Tracking Scale (S-STS).

### 1.2. Study Assessments and Procedures

A tabular summary of the schedule of study assessments and procedures by visit is provided in the Schedule of Assessments and Visits (Synopsis Table 1). A more detailed description of the assessments at each visit is provided elsewhere in this Example under ‘Schedule of Evaluations and Procedures’.

### 2. Selection and Withdrawal of Patients

Patients enrolled in this study must have a diagnosis of probable Alzheimer's disease and must present with clinically significant, moderate-to-severe agitation secondary to Alzheimer's disease. The diagnosis of probable Alzheimer's disease is based on the ‘2011 Diagnostic Guidelines for Alzheimer's Disease’ issued by the National Institute on Aging (NIA)-Alzheimer's Association (AA) workgroups. Neither Alzheimer's disease nor agitation should be explainable by delirium, substance use and/or major psychiatric disorders.

The provisional consensus definition of agitation in patients with cognitive disorders developed by the Agitation Definition Work Group (ADWG) from the International Psychogeriatric Association (IPA) is used to select study patients. This proposed definition is limited to patients with cognitive impairment and requires: (a) evidence of emotional distress; (b) 1 of 3 observable types of behaviors: excessive motor activity, verbal aggression, or physical aggression; (c) that the behavior causes excess disability; and (d) that the behaviors cannot be solely attributable to a suboptimal care environment or other disorder such as a psychiatric illness, a medical illness, or effects of a substance.

Eligible patients must have clinically significant, moderate-to-severe agitation for at least 2 weeks prior to Screening that interferes with daily routine per the Investigator's judgment, and who require pharmacotherapy for the treatment of agitation per the Investigator's judgment after an evaluation of reversible factors and a course of nonpharmacological interventions.

An NPI-AA score of ≥4 and MMSE score of 8 to 24 (inclusive) at Screening and Baseline are required for study participation.

Eligible patients are to have otherwise acceptable and stable general health as required by the study protocol and documented by medical history, physical and neurological examination, ECG, and clinical laboratory examinations.

Eligible patients must have a caregiver who is able and willing to comply with all required study procedures, ensuring that the patient attends all study visits and takes the study drug as instructed, including adherence to not administering any prohibited medications during the course of the study. Caregivers are also instructed to record the daily number of capsules taken and the time of administration in the patient Diary Card. In addition, caregivers are responsible for reporting any changes in patient's status, including adverse events and standard of care setting (e.g., becoming a resident in an assisted living facility), as well as providing their impression and assessment regarding the investigational treatment to the study team at the Investigator's site. A CMAI caregiver diary is provided to be used by the caregiver to support reporting of behaviors during the CMAI interview process. In order to qualify as a reliable informant (i.e., caregiver) capable of assessing changes in the patient's condition during this study, the individual must spend a minimum of 2 hours with the patient per day for 4 days per week. In addition, this individual should remain as the patient's caregiver throughout the study.

The complete list of inclusion and exclusion criteria for this study are provided in the following sections.

### 2.1. Patient Inclusion Criteria

1. Males and females 50 to 90 years of age (inclusive) at the time of informed consent.
2. Diagnosis of probable Alzheimer's disease according to the 2011 NIA-AA working groups criteria. Either outpatients or residents of an assisted living facility, a skilled nursing home, a dementia unit, or any other type of facility providing long-term care.
3. MMSE score between 8 and 24 (inclusive) at Screening and Baseline.
4. Patient has clinically significant, moderate-to-severe agitation for at least 2 weeks prior to Screening that interferes with daily routine per the Investigator's judgment.
5. Patients who require pharmacotherapy for the treatment of agitation per the Investigator's judgment, after:
   An evaluation of reversible factors (e.g., pain, infection, or polypharmacy), and
   A course of nonpharmacological interventions (e.g., redirecting behavior, group activities, music therapy).
6. Diagnosis of agitation must meet the International Psychogeriatric Association (IPA) provisional definition of agitation.
7. NPI-AA total score (frequency×severity) must be ≥4 at Screening and Baseline.
8. Patient has stable cardiac, pulmonary, hepatic, and renal function per the Investigator's judgment.
9. No clinically significant findings on the Screening ECGs based on central review and on the Baseline predose ECG based on the machine read and Investigator's evaluation.
10. Women who are of childbearing potential and are sexually active must use an effective method of birth control for at least 1 month prior to the Baseline, during participation in the study, and for at least 30 days after the last dose of study drug. The following requirements must be met:
   Women who are of childbearing potential must use 2 of the following precautions in order to minimize the risk of failure of 1 method of birth control: vasectomy, tubal ligation, vaginal diaphragm, intrauterine device, birth control pills, birth control depot injection, birth control implant, or condom with spermicide or sponge with spermicide. Periodic abstinence (e.g., calendar, ovulation, symptothermal, post-ovulation methods), declaration of abstinence for the duration of exposure to study drug, or withdrawal are not acceptable methods of contraception.
   Women who are sterile (i.e., had an oophorectomy and/or hysterectomy), postmenopausal (defined as 12 consecutive months with no menses without an alternative medical cause), or practice true abstinence (when this method is in line with the preferred and usual lifestyle of the patient) are exempt from this requirement.
   Women who are lactating, pregnant, or plan to become pregnant are not eligible for participation in the study.
11. For restricted and prohibited concomitant medications, patients willing and able to meet all protocol requirements for duration of stability or washout prior to study entry and during the study (see Table 3 Restricted and Prohibited Concomitant Medications and Appendix 1 Prohibited Concomitant Medications).

### 2.2. Patient Exclusion Criteria

1. Caregiver is unwilling or unable, in the opinion of the Investigator, to comply with study instructions.
2. Patient has dementia predominantly of non-Alzheimer's type (e.g., vascular dementia, frontotemporal dementia, Parkinson's disease, substance-induced dementia).
3. Patients with symptoms of agitation that are not secondary to Alzheimer's dementia (e.g., secondary to pain, other psychiatric disorder, or delirium).
4. Patients who have been diagnosed with an Axis 1 disorder (Diagnostic and Statistical Manual of Mental Disorders, 5th Edition, Text Revision [DSM-5] criteria) including, but not limited to.
   Schizophrenia, schizoaffective disorder, or other psychotic disorders not related to dementia
   Bipolar I or II disorder, bipolar disorder not otherwise specified
   Current Major Depressive Episode: Patients with a history of major depressive disorder, that is currently not symptomatic, are eligible. Patients currently on a stable dose(s) of allowed antidepressant medication(s) for at least 3 months prior to the Screening visit are eligible.
5. Patients with myasthenia gravis (contraindication for quinidine).
6. Patients with any personal history of complete heart block, QTc prolongation, or torsades de pointes.
   Screening and Baseline predose QT interval corrected for heart rate using the Fridericia's formula (QTcF) of >450 msec for males and >470 msec for females unless due to ventricular pacing (See Section 6.1.5).
Screening ECGs are based on central review. Baseline predose ECG is based on the machine read and Investigator's evaluation; if the QTcF result from the machine read is exclusionary, do not administer study drug and please contact a Medical Monitor.
   Presence of premature ventricular contractions (PVCs) as evaluated by a central reader and deemed clinically significant by the Investigator.
7. Patients with any family history of congenital QT interval prolongation syndrome.
8. Patients with known hypersensitivity to DM, Q, opiate drugs (codeine, etc.), or any other ingredient of the study drug.
9. Patients who have ever received DM co-administered with Q or d6-DM co-administered with Q.
10. Patients who would be likely to require a prohibited concomitant medication during the study (see Table 3, Restricted and Prohibited Concomitant Medications and Appendix 1 Prohibited Concomitant Medications).
11. Patients with co-existent clinically significant or unstable systemic diseases that could confound the interpretation of the safety results of the study (e.g., malignancy [except skin basal-cell carcinoma], poorly controlled diabetes, poorly controlled hypertension, unstable pulmonary, renal or hepatic disease, unstable ischemic cardiac disease, dilated cardiomyopathy, or unstable valvular heart disease). Certain other nonmetastatic cancer may be allowed. Each case is to be evaluated individually with a Medical Monitor.
12. Patients who are currently participating in or who have participated in other interventional (drug or device) clinical study, or found to be a “Virtually Certain” match in Clinical Trial Subject Database (CTSdatabase) with a patient who has participated in another interventional drug or device study within 30 days of Baseline.
13. Patients with history of postural syncope or any history of unexplained syncope (evaluated on a case-by-case basis) within 12 months of Baseline.
14. Patients with a history of substance and/or alcohol abuse within 12 months of Baseline.
15. Patients determined to have a high imminent risk of falls during the study based on a clinical evaluation by the Investigator.
16. Patients with evidence of serious risk of suicide at Screening and Baseline based on the Sheehan Suicidality Tracking Scale (S-STS), i.e., a score of 3 or 4 on any one question 2 through 6 or 11, or a score of 2 or higher on any one questions 1a, 7 through 10, or 12, or who in the opinion of the Investigator present a serious risk of suicide.
17. Patients who, in the opinion of the Investigator, Medical Monitor, or sponsor, should not participate in the study.

### 2.3. Patient Withdrawal Criteria

Patients and caregivers are advised verbally and in the written ICF that they have the right to withdraw from the study at any time without prejudice or loss of benefits to which they are otherwise entitled. The Investigator or sponsor may discontinue a patient from the study in the event of an intercurrent illness, adverse event, other reasons concerning the health or well-being of the patient, or in the case of lack of cooperation, noncompliance, protocol violation, or other administrative reasons. If a patient does not return for a scheduled visit, every effort should be made to contact the patient. Regardless of the circumstance, every effort should be made to document patient outcome, if possible. The Investigator should inquire about the reason for withdrawal, request the caregiver return all unused study drug, and follow-up with the patient regarding any unresolved adverse events.

In addition, patients who present at any time after the Baseline visit (Day 1) with a persistent QTc interval (QTcF)>500 msec (unless due to ventricular pacing) or a persistent QTcF interval change from the predose Baseline ECG of >60 msec, that is confirmed by the central ECG reader, are withdrawn from the study after consultation with a Medical Monitor. The QTcF values are assessed for clinical significance and recorded.

Patients who terminate early from the study have an ET visit to complete the Visit 8 (Day 85/ET) assessments and a Follow-up visit 30 days after the last dose of study drug for select efficacy and safety assessments. If a patient terminates early from the study, the investigator and site personnel should make every effort to have the patient and caregiver return to the clinic for the ET visit and the Follow-up visit.

### 3. Treatment of Patients

### 3.1. Description of Study Drug

Study drug is provided as opaque hard gelatin capsules with a purple cap and white body. Each capsule contains one of the following:
AVP-786-18 capsules: 18 mg of d6-DM and 4.9 mg of Q (USP; EP)
AVP-786-42.63 capsules. 42.63 mg of d6-DM and 4.9 mg of Q (USP; EP)

Drug supplies are provided to the site in double-blind, individual, prelabeled blister cards.

### 3.2. Concomitant Medications and Nondrug Therapies

At each visit, caregivers are queried as to whether or not the patient has taken any concomitant medications and, if so, the Investigator records the medications taken and the reasons for their use.

AVP-786 contains quinidine which is a P-glycoprotein inhibitor. Concomitant administration of quinidine with digoxin, a P-glycoprotein substrate, results in serum digoxin levels that may be as much as doubled. Plasma digoxin concentrations should be closely monitored in patients taking digoxin concomitantly and dose reduced, as necessary.

In cases of prodrugs whose actions are mediated by the CYP2D6-produced metabolites (for example, codeine and hydrocodone, whose analgesic and antitussive effects appear to be mediated by morphine and hydromorphone, respectively), it may not be possible to achieve the desired clinical benefits in the presence of AVP-786 due to quinidine-mediated inhibition of CYP2D6. Alternative treatment should be considered.

For patients who terminate early, the conditions of use for allowed concomitant medications and nondrug therapies or prohibited medications do not apply between the ET visit and the Follow-up visit. Any use of medications during this period are at the discretion of the Investigator. Patients should allow at least 14 days after stopping study drug before starting a monoamine oxidase inhibitor (MAOI).

### 3.2.1. Restricted and Prohibited Concomitant Medications

Psychotropic concomitant medications that are either allowed with certain restrictions or prohibited are listed in Table 3. A detailed list of prohibited concomitant medications that may result in significant drug-drug interactions is provided in Appendix 1.

TABLE 3
Restricted and Prohibited Concomitant Medications

| | | | During Double-Blind |
|---|---|---|---|
| Medication | | Prior to Study Entry | Treatment Period |
| 1. | Medications to treat | Allowed provided that the dose lias | Should remain on the same dose |
| | Alzheimer’s disease (e.g., | been stable for at least 3 months prior | throughout the duration of the |
| | cholinesterase inhibitors, | to the Screening visit, AND there is no | study, except when medically |
| | memantine, or other | change in dose or discontinuation | indicated due to a change in the |
| | cognitive enhancers approved | within the same time period | underlying medical condition |
| | in the respective country) | | |
| 2. | Atypical Antipsychotics | Allowed provided that the dose has | Should remain on the same dose |
| | except clozapine | been stable for at least 1 month prior to | throughout the duration of the |
| | | the Screening visit, AND there is no | study |
| | | change in dose or discontinuation | |
| | | within the same time period | |
| | Clozapine | Not allowed within 1 month prior to the | Prohibited |
| | | Screening visit | |
| | Typical Antipsychotics | Not allowed within 1 month prior to the | Prohibited |
| | | Screening visit | |
| 3. | Antidepressants | Allowed provided that the dose has | Should remain on the same dose |
| | elect antidepressants listed | been stable for at least 3 months prior | throughout the duration of the |
| | below are restricted or | to the Screening visit and is within the | study |
| | prohibited | range specified in the prescribing | |
| | | information for that medication, AND | |
| | | there is no change in dose or | |
| | | discontinuation within the same time | |
| | | period | |
| | Paroxetine | Allowed with a maximum dose of | Should remain on the same dose |
| | | 10 mg/day | throughout the duration of the |
| | | | study |
| | Trazodone | Allowed with a maximum dose of | Should remain on the same dose |
| | | 50 mg/day | throughout the duration of the |
| | | | study |
| | Nefazodone | Not allowed within 3 months prior to | Prohibited |
| | | the Screening visit | |
| | Tricyclic Antidepressants | Not allowed witinn 3 months prior to | Prohibited |
| | | the Screening visit | |
| | Monoamine Oxidase | Not allowed within 3 months prior to | Prohibited |
| | Inhibitors (MAOI) | the Screening visit | Patients should allow at least |
| | | | 14 days after stopping study |
| | | | drug before starting an MAOI |
| 4. | Benzodiazepines | Not allowed within 1 month prior to the | Prohibited |
| | | Screening visit. | |
| 5. | Hypnotic Sleep Agents | Allowed if the dose of the sleep agent | Should remain on the same dose |
| | (e.g., zolpidem, zaleplon, | for insomnia was taken on a regular | and at the same frequency |
| | zopiclone, eszopicione, or | basis for at least 1 month prior to the | throughout the duration of the |
| | other sleep agent approved in | Screening visit, AND there is no | study |
| | the respective country) | change in dose or frequency, or | |
| | | discontinuation in the same time period | |
| 6. | Other allowed psychotropic | Allowed provided that the dose has | Should remain on the same dose |
| | medications that may impact | been stable for at least 1 month prior to | throughout the duration of the |
| | agitation (e.g., mood | the Screening visit, AND there is no | study |
| | stabilizers, antiepileptics) | change in dose or discontinuation | |
| | select medications listed | within the same time period | |
| | below are prohibited | | |
| | Atomoxetine, carbamazepine, | Not allowed within 1 month prior to the | Prohibited |
| | fosphenytoin, pentobarbital, | Screening visit | |
| | phenobarbital, phenytoin, | | |
| | primidone | | |
| 7 . | Other prohibited concomitant | Not allowed within 2 weeks or | Prohibited |
| | medications (see Appendix 1) | 5 half-lives (whichever is longer) prior | |
| | | to the Baseline visit | |

### 4. Study Drug Materials and Management

### 4.1. Study Drug Composition

Each capsule of study drug contains one of the following:
AVP-786-18/4.9: 18 mg of d6-DM and 4.9 mg of Q (USP; EP)
AVP-786-42.63/4.9: 42.63 mg of d6-DM and 4.9 mg of Q (USP; EP)

### 5. Assessment of Efficacy

Whenever possible, each patient and caregiver should have the rating scales administered by the same raters throughout the study, for consistency of ratings. The following scales MUST be administered by the same rater at each visit: CMAI, CGIS-Agitation, CGIC-Agitation, and NPI.

### 5.1. Cohen-Mansfield Agitation Inventory (CMAI)

The CMAI is used as the primary efficacy measure in this study. The CMAI (long-form version) is used to assess the frequency of manifestations of agitated behaviors in elderly persons. It consists of 29 agitated behaviors that are further categorized into distinct agitation syndromes, also known as CMAI factors of agitation. These distinct agitation syndromes include: aggressive behavior, physically nonaggressive behavior, and verbally agitated behavior. Scores for the 3 dimensions Factor 1, Factor 2, and Factor 3 are derived based on the factor structure described by Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998 and in more detail elsewhere herein. Each of the 29 items is rated on a 7-point scale of frequency (1=never, 2=less than once a week but still occurring, 3=once or twice a week, 4=several times a week, 5=once or twice a day, 6=several times a day, 7=several times an hour). The ratings are based on the 2 weeks preceding assessment of the CMAI.

The CMAI is assessed at Screening (Day −28 to Day −1), Baseline (Day 1), Visit 2 (Day 8), Visit 3 (Day 15), Visit 4 (Day 29), Visit 5 (Day 43), Visit 6 (Day 57), Visit 7 (Day 71), Visit 8 (Day 85/ET), and the Follow-up visit (30 days postdose). The CMAI must be administered by the same rater at each visit.

### 5.2. Clinical Global Impression of Severity of Illness-Agitation (CGIS-Agitation)

The CGIS is an observer-rated scale that measures illness severity and is one of the most widely used brief assessment tools in psychiatry research.

The Early Clinical Drug Evaluation Unit version of the CGIS is the most widely used format of this validated tool, and asks that the clinician rate the patient relative to their past experience with other patients with the same diagnosis, with or without collateral information. The CGIS has proved to be a robust measure of efficacy in many clinical drug trials and is easy and quick to administer, provided that the clinician knows the patient well.

Reliability and validity of CGI have been tested in multiple studies, including patients with dementia, schizophrenia and affective disorders. Overall, CGI showed high correlation (r: ˜90%) with other assessment instruments and it has also shown positive significant relationships and concurrent validity with other clinician's rating. In addition, the scale has good sensitivity to change over time.

The CGIS-Agitation is a 7-point (1-7) scale (1=normal, not at all ill; 7=among the most extremely ill patients) and assesses severity of agitation in this study. The CGIS-Agitation is assessed at Screening (Day −28 to Day −1), Baseline (Day 1), Visit 2 (Day 8), Visit 3 (Day 15), Visit 4 (Day 29), Visit 5 (Day 43), Visit 6 (Day 57), Visit 7 (Day 71), Visit 8 (Day 85/ET), and the Follow-up visit (30 days postdose). The CGIS-Agitation must be administered by the same rater at each visit.

### 5.3. Clinical Global Impression of Change-Agitation (CGIC-Agitation)

The CGIC-Agitation is a 7-point (1-7) scale (1=very much improved; 7=very much worse) that assesses the change in the severity of agitation in this study. The CGIC-Agitation evaluation is conducted at Visit 2 (Day 8), Visit 3 (Day 15), Visit 4 (Day 29), Visit 5 (Day 43), Visit 6 (Day 57), Visit 7 (Day 71), and Visit 8 (Day 85/ET). The CGIC-Agitation must be administered by the same rater at each visit.

### 5.4. Neuropsychiatric Inventory (NPI)

The NPI is a validated clinical instrument for evaluating psychopathology in a variety of disease settings, including dementia. The NPI is a retrospective caregiver-informant interview covering 12 neuropsychiatric symptom domains: delusions, hallucinations, agitation/aggression, depression/dysphoria, anxiety, elation/euphoria, apathy/indifference, disinhibition, irritability/lability, aberrant motor behavior, sleep and nighttime behavioral disorders, and appetite/eating disorders. Neuropsychiatric manifestations within a domain are collectively rated by the caregiver in terms of both frequency (1 to 4) and severity (1 to 3), yielding a composite symptom domain score (frequency×severity). Caregiver distress is rated for each positive neuropsychiatric symptom domain on a scale anchored by scores of 0 (not distressing at all) to 5 (extremely distressing).

The NPI is administered to the patient's caregiver at Baseline (Day 1), Visit 2 (Day 8), Visit 5 (Day 43), Visit 7 (Day 71), and Visit 8 (Day 85/ET). Only the Agitation/Aggression domain of the NPI (NPI-AA) is administered to the patient's caregiver at Screening (Day −28 to Day −1), Visit 3 (Day 15), Visit 4 (Day 29), and Visit 6 (Day 57). The recall period is 2 weeks for all the visits. The NPI must be administered by the same rater at each visit. The NPI nursing-home version (NPI-NH) is used for patients from in-patient or assisted living facilities.

### 5.5. EuroQol 5-Dimension 5-Level (EQ-5D-5L)

The EQ-5D-5L is a generic questionnaire measuring health-related quality of life and consists of a descriptive system and the EuroQol Visual Analogue Scale (EQ VAS). The descriptive system comprises 5 dimensions (mobility, self-care, usual activities, pain/discomfort, and anxiety/depression) and each dimension has 5 levels: no problems, slight problems, moderate problems, severe problems, and extreme problems. The EQ VAS records the respondent's self-rated health on a vertical, visual analogue scale where the endpoints are labeled ‘Best imaginable health state’ and ‘Worst imaginable health state’. This information can be used as a quantitative measure of health outcome as judged by the individual respondents. There are 2 versions of the EQ-5D-5L, a version rated by the patient and a version (EQ-5D-5L-proxy) rated by caregiver. The patient version is rated only by patients with an MMSE score of ≥10 at the Baseline visit.

The EQ-5D-5L-proxy (and EQ-5D-5L for patients with MMSE≥10) is assessed at Baseline (Day 1) and Visit 8 (Day 85/ET).

### 5.6. Resource Utilization in Dementia (RUD) Lite

The RUD is used to calculate healthcare costs associated with dementia. It evaluates dementia patients' utilization of formal and informal healthcare resources, including hospitalizations and doctor visits, living assistance, and time spent by nonprofessional caregivers. Within the context of clinical trials, the RUD is often used to determine the cost effectiveness of new pharmaceutical treatments.

The RUD is administered as a semi-structured interview with the patient's primary caregiver, and contains 2 sections; one focusing on caregiver impact (loss of work and leisure time incurred by caregiver) and the other focusing on the patient's use of healthcare resources. The total healthcare costs associated with the patient's dementia can be estimated by multiplying the number of units used (e.g., hours of caregiver time, visits to doctors, nights in accommodation) by the corresponding unit price vector.

The RUD-Lite (RUD 5.0) is a shorter version of the RUD developed to reduce the interview burden on caregivers. Questions related to caregiver resource use (e.g., work status, respite or hospital care, social services, day care, or drug use), which in general is low for caregivers, have been removed from the RUD-Lite.

The RUD-Lite is assessed at Baseline (Day 1) and Visit 8 (Day 85/ET).

### 6. Assessment of Safety

### 6.1. Safety Parameters

### 6.1.1. Adverse and Serious Adverse Events

### 6.1.1.1. Definition of Adverse Events

An adverse event (AE) is any untoward medical occurrence or unintended change (e.g., physical, psychological, or behavioral), including inter-current illness, whether considered related to study drug or not. An AE can therefore be any unfavorable and unintended sign (including any clinically significant abnormal laboratory finding, for example), symptom, or disease temporally associated with the use of a study drug, whether or not considered related to the study drug. Changes associated with normal growth and development that do not vary in frequency or magnitude from that ordinarily anticipated clinically are not AEs (e.g., onset of menstruation occurring at a physiologically appropriate time).

Clinical AEs should be described by diagnosis and not by symptoms when possible (e.g., cold or seasonal allergies, instead of runny nose).

An overdose is a deliberate or inadvertent administration of a treatment at a dose higher than specified in the protocol and higher than known therapeutic doses. It must be reported irrespective of outcome, even if toxic effects were not observed.

AEs are graded on a 3-point severity scale and reported in detail as indicated on the eCRF:

Mild; easily tolerated, causing minimal discomfort and not interfering with normal everyday activities
Moderate: sufficiently discomforting to interfere with normal everyday activities
Severe: incapacitating and/or preventing normal everyday activities

The relationship of each AE to study drug should be determined by the investigator using the following explanations:
Not related: the event is clearly related to other factors such as the patient's clinical state, therapeutic interventions, or concomitant medications administered to the patient
Unlikely related: the event is most likely produced by other factors such as the patient's clinical state, therapeutic interventions, or concomitant medications administered to the patient; and does not follow a known response pattern to the study medication
Possibly related: the event follows a reasonable temporal sequence from the time of study drug administration; and/or follows a known response pattern to the study drug; but could have been produced by other factors such as the patient's clinical state, therapeutic interventions, or concomitant medications administered to the patient
Related: the event follows a reasonable temporal sequence from the time of study drug administration; and follows a known response pattern to the study drug; and cannot be reasonably explained by other factors such as the patient's clinical state, therapeutic interventions, or concomitant medications administered to the patient

### 6.1.1.2. Serious Adverse Event

A serious adverse event (SAE) is any AE occurring at any dose that results in any of the following outcomes:
1. Death
2. Life-threatening experience (one that places the patient, in the view of the initial reporter, at immediate risk of death from the AE as it occurred, i.e., it does not include an AE that, had it occurred in a more severe form, might have caused death)
3. Persistent or significant disability/incapacity (disability is a substantial disruption of a person's ability to conduct normal life functions)
4. In-patient hospitalization or prolongation of hospitalization
5. Congenital anomaly/birth defect

Important medical events that may not result in death, or be life-threatening, or require hospitalization may be considered an SAE when, based upon appropriate medical judgment, they may jeopardize the patient or require medical or surgical intervention to prevent one of the outcomes listed in the definition.

The terms “cancer” and “overdose” are not necessarily considered SAEs, but if a patient experiences cancer or overdose, they are still reportable as AEs.

Pregnancy is not considered to be an AE or an SAE, but all reported pregnancies occurring during the study are reported on the Pregnancy and Breastfeeding Exposure Form (PBEF). The site should follow-up each trimester with the patient/partner until the final outcome is known (i.e., normal delivery, abnormal delivery, spontaneous/voluntary/therapeutic abortion). Should a complication occur that meets the requirements for an AE or SAE, it must be reported within 24 hours of awareness. Patients who are pregnant or likely to become pregnant are excluded from this study. In the event a patient becomes pregnant during the study, study drug must be discontinued, a pregnancy report form must be completed to capture potential drug exposure during pregnancy, and the pregnancy must be reported within 24 hours of awareness.

A pregnancy report form must also be completed in the event that the partner of child-bearing potential of a male patient in the study becomes pregnant within 30 days after his last dose of study drug or study completion, whichever is greater.

The term ‘severe’ is a measure of intensity; thus, a severe AE is not necessarily serious. For example, nausea of several hours duration may be rated as severe but may not be clinically serious.

### 6.1.2. Physical and Neurological Examinations

Physical and neurological examinations are performed at Screening (Day −28 to Day −1) and Visit 8 (Day 85/ET). The physical examination includes assessments of head, eyes, ears, nose, throat, lymph nodes, skin, extremities, respiratory, gastrointestinal, musculoskeletal, cardiovascular, and nervous systems. The neurological examination includes assessments of mental status, cranial nerves, motor system, reflexes, coordination, gait and station, and sensory system. The physical and neurological examinations should be performed by the same person each time, whenever possible.

Physical and neurological examination abnormalities determined by the investigator to be clinically significant at Screening should be recorded as medical history.

Any clinically significant changes in physical and neurological examination findings from the screening examination should be recorded as AEs.

### 6.1.3. Vital Signs

Orthostatic blood pressure (BP) and heart rate (HR) measurements are performed at all clinic visits, except the Follow-up visit. Supine BP and HR are measured after a patient has rested for at least 5 minutes in the supine position. Each measurement is taken twice in the same position and recorded. After the measurement of supine BP and HR, the patient stands still for up to 3 minutes and a single measurement of standing BP and HR is recorded within 1 to 3 minutes of standing.

Respiratory rate (breaths/minute) and body temperature (° F./° C.) are assessed at all clinic visits.

### 6.1.4. Weight and Height

Height and weight are measured at Baseline (Day 1); only weight is measured at Visit 8 (Day 85/ET).

### 6.1.5. Electrocardiogram (ECG)

A resting 12-lead ECG is performed at Screening, Baseline, Visit 2 (Day 8), Visit 5 (Day 43), and Visit 8 (Day 85/ET). At Screening, 3 ECGs are performed (e.g., one after the other). At Baseline (Day 1) and Visit 2 (Day 8), 2 ECGs are performed; one predose prior to study drug dosing and one postdose 1 to 1.5 hours after study drug dosing. An ECG is performed at any time at Visit 5 (Day 43) and Visit 8 (Day 85/ET).

ECG equipment is provided by the central reader. ECG data is recorded at the study center and includes general findings, heart rate (beats/minute), QRS complex, PR and QTc intervals (milliseconds). Results are provided by the central reader to the investigators within 24 hours. ECG abnormalities present at Screening are recorded as medical history. Any changes from the ECG status at Screening visit that are deemed to be clinically significant by the investigator should be captured as AEs. Any clinically significant abnormal ECG should be discussed with a Medical Monitor and, if necessary, be repeated within a 1-week period.

For eligibility to enroll in the study, the QTcF assessment of the 3 ECGs conducted at Screening is based on the central review. A patient is excluded if 2 of the 3 Screening ECGs have a QTcF>450 msec in males and >470 msec in females, unless due to ventricular pacing. If only 1 Screening ECG has a QTcF>450 msec in males and >470 msec in females, which is not reproduced in either of the other 2 Screening ECGs, then the patient may be eligible for the study. The assessment of ECGs conducted at Baseline is based on the machine read and investigator evaluation of the read. If the Baseline predose ECG QTcF result from the machine read is exclusionary, the patient should not be dosed and a Medical Monitor should be consulted.

### 6.1.6. Clinical Laboratory Assessments

Unless otherwise specified, the following clinical laboratory assessments are to be performed at Screening (Day −28 to Day −1), Visit 5 (Day 43), and Visit 8 (Day 85/ET):
Blood chemistry (calcium, magnesium, phosphorus, glucose, sodium, potassium, chloride, carbon dioxide, blood urea nitrogen, serum creatinine, uric acid, albumin, total bilirubin, alkaline phosphatase, lactate dehydrogenase, aspartate aminotransferase/serum glutamic oxaloacetic transaminase, alanine aminotransferase/serum glutamic pyruvic transaminase, creatine kinase, gamma-glutamyl transferase, triglycerides, total protein, and total cholesterol)
Hematology (red blood cell count, hemoglobin, hematocrit, white blood cell [WBC] count, neutrophils, bands, lymphocytes, monocytes, eosinophils, basophils, platelet count, and morphology)
Urinalysis (pH, specific gravity, protein, glucose, ketones, blood, leucocyte esterase, nitrates, and microscopic appearance)
Thyroid function tests (TSH, and reflex T3 and T4 if TSH is abnormal) at Screening visit only
Glycosylated hemoglobin (HbA1c) test at the Screening visit and Visit 8 (Day 85) only
CYP2D6 genotyping at Visit 2 (Day 8) only
Amyloid p biomarker at Visit 2 (Day 8) only
   Any patients with clinically significant abnormal laboratory test results may be required by a Medical Monitor to have a repeat test 1 week later or earlier, if medically indicated. Clinically significant laboratory abnormalities may be a basis for exclusion from study entry.

### 6.1.7. Mini Mental State Examination (MMSE)

The MMSE is a brief 30-point questionnaire test that is used to screen for cognitive impairment. It is commonly used in medicine to screen for dementia. It is also used to estimate the severity of cognitive impairment at a specific time and to follow the course of cognitive changes in an individual over time, thus making it an effective way to document an individual's response to treatment. The MMSE scale comprises 11 questions or simple tasks concerning orientation, memory, attention, and language to evaluate the patient's cognitive state. The MMSE total score ranges from 0 to 30, with higher scores indicating better cognitive function. It requires only 5 to 10 minutes for a trained rater to administer it.

The MMSE is assessed at Screening (Day −28 to Day −1), Baseline (Day 1), and Visit 8 (Day 85).

### 6.1.8. Epworth Sleepiness Scale (ESS)

The ESS is an 8-item questionnaire that is used to measure sleepiness by rating the probability of falling asleep on 8 different situations that most people engage in during the day. The questions are rated on a 4-point scale (0 to 3) where 0=would never doze, 1=slight chance of dozing, 2=moderate chance of dozing, and 3=high chance of dozing. A total score of 0 to 9 is considered to be normal.

The ESS is assessed at Baseline (Day 1) and Visit 8 (Day 85/ET) for patients with an MMSE score of ≥10 at the Baseline visit.

### 6.1.9. Sheehan Suicidality Tracking Scale (S-STS)

The S-STS is a prospective scale that assesses treatment-emergent suicidal thoughts and behaviors. Each item of the S-STS is scored on a 5-point Likert scale (0=not at all; 1=a little; 2=moderate; 3=very; and 4=extremely). The S-STS can be analyzed as individual item scores, suicidal ideation subscale score, suicidal behavior subscale score, or total score. For the Screening visit, the timeframe for the items on the scale is ‘in the past 6 months’ and for all other visits it is ‘since last visit’.

The S-STS is assessed at Screening (Day −28 to Day −1), Baseline (Day 1), and Visit 8 (Day 85/ET). Any change in the S-STS score indicating the presence of suicidality should be evaluated by the investigator and reported to a Medical Monitor.

### 6.1.10. Assessment of Risk of Falls for Eligibility

### 6.1.10.1. Timed Up and go (TUG) Test

The TUG test measures the time (in seconds) taken for an individual to stand up from a standard armchair, walk 3 meters, turn, walk back to the chair and sit down. It is a commonly used scale for measuring functional mobility and risk of falls. The TUG test is performed only at Screening (Day −28 to Day −1) to assess the risk of falls for the purpose of eligibility for the study.

### 6.1.11. Hachinski Ischemic Scale (Rosen Modification)

The Rosen-modified Hachinski Ischemic Scale assesses whether a patient's dementia is likely due to vascular causes by the response to 8 questions: abrupt onset, stepwise deterioration, somatic complaints, emotional incontinence, history of hypertension, history of stroke, focal neurologic signs, and focal neurologic symptoms. The total score ranges from 0 to 12, with higher scores indicating a greater risk of vascular dementia. The Rosen-modified Hachinski Ischemic Scale is completed at the Screening visit to assess the risk of vascular dementia and eligibility for the study by the same physician who performs the neurological examination.

### 7. Schedule of Evaluations and Procedures

A schedule of evaluations and procedures by visit is provided in Table 1.

### 7.1. Description of Study Procedures

### 7.1.1. Screening Visit (Days −28 to −1)

The following procedures are performed at Screening (within 28 days prior to Day 1). The screening period may be extended after discussion with and approval by a Medical Monitor. In the event that a patient is rescreened for enrollment, new informed consent and/or assent documents must be signed, new patient number assigned, and all screening procedures repeated.
1. The Investigator provides the patients, authorized representatives, and/or their caregivers with informed consent and/or assent documents and explains the rationale for the study, providing ample time for participants, authorized representatives, and/or caregivers to ask questions.
2. Review inclusion/exclusion criteria (protocol eligibility form completed by site).
3. Medical, psychiatric, and neurological history are reviewed and recorded, including patient demographics, any prior and concomitant medications (including over-the-counter [OTC], vitamins, and supplements), nondrug therapies, and any nonpharmacological interventions for the treatment of agitation.
4. Risk assessment for falls are performed (worksheet and TUG test).
5. Vital signs are measured and recorded.
6. Physical and neurological examination is performed.
7. Three resting 12-lead ECGs are performed.
8. A blood and urine specimen is collected for safety laboratory assessments (including thyroid function tests and Hb A1c).
9. A urine pregnancy test is performed for women of childbearing potential only.
10. The caregiver is queried regarding AEs.
11. The following assessments are completed (the CMAI and NPI-AA should be administered before the CGIS-Agitation):
   CMAI
   NPI-AA domain; a score of ≥4 is required for study entry
   CGIS-Agitation
   MMSE; a score between 8 and 24 (inclusive) is required for study entry
   S-STS
12. Register the Screening visit in IWRS.

Following screening procedures for assessment of inclusion and exclusion criteria, the site will complete a protocol eligibility form to be reviewed by a Medical Monitor for approval prior to participation in the study. Patients deemed eligible by the Investigator and a Medical Monitor will proceed to the Baseline visit of the study.

Patients who have ECG or laboratory test results outside of the reference normal range that the investigator considers to be clinically significant and may put the patient at a higher risk for study participation, will not be enrolled.

### 7.1.2. Baseline Visit (Day 1)

The Baseline visit (Day 1) should occur in the morning. The following procedures are performed.

Before Dosing:
1. Review inclusion/exclusion criteria.
2. Vital signs are measured and recorded.
3. Weight and height are measured and recorded.
4. A resting 12-lead ECG is performed (predose).
5. A urine pregnancy test is performed for patients of childbearing potential only.
6. The caregiver is queried regarding AEs, concomitant medications (including OTC, vitamins, and supplements), nondrug therapies, and any nonpharmacological interventions for the treatment of agitation.
7. The following assessments are completed (the CMAI and NPI should be administered before the CGIS-Agitation):
   CMAI
   NPI
   CGIS-Agitation
   EQ-5D-5L
   RUD-Lite
   MMSE
   ESS (only for patients with an MMSE score of ≥10 at Baseline)
   S-STS

Patients will proceed with the Baseline visit once it is determined that they satisfy all of the inclusion and none of the exclusion criteria (on the basis of the Screening and Baseline assessments described above) and are assigned with a blister card kit number via IWRS.

After Dosing:
1. A resting 12-lead ECG is performed (1 to 1.5 hours postdose).
2. The caregiver is queried regarding AEs.
3. Patient Diary Card and a blister card (1-week blister card) are dispensed.

### 7.1.3. Visit 2 (Day 8±3-Day Window)

Visit 2 (Day 8) should occur in the morning prior to the morning dose of study drug. The following procedures are performed.

Before Dosing:
1. Vital signs are measured and recorded.
2. The caregiver is queried regarding AEs, concomitant medications (including OTC, vitamins, and supplements), nondrug therapies, and any nonpharmacological interventions for the treatment of agitation.
3. A resting 12-lead ECG is performed (predose).
4. The following assessments are completed (the CMAI and NPI should be administered before the CGIS-Agitation and CGIC-Agitation):
   CMAI
   NPI
   CGIS-Agitation
   CGIC-Agitation
5. Register the study visit in IWRS and a blister card kit number are assigned to patient (1-week blister card).

After Dosing:
1. A resting 12-lead ECG is performed (1 to 1.5 hours postdose).
2. A blood and urine sample is collected for PK assessments (1 to 4 hours postdose).
3. A blood sample is collected for CYP2D6 genotyping.
4. A blood sample is collected for amyloid p biomarker.
5. Unused study drug is accounted for compliance, and a new blister card is dispensed (1-week blister card).
6. Patient's Diary Card is reviewed for compliance and returned to the patient.

### 7.1.4. Visit 3 (Day 15±3-Day Window)

Visit 3 (Day 15) should occur in the morning. The morning dose of study drug can be administered at home any time prior to the visit; the time of dosing should be noted by the patient/caregiver.

The following procedures are performed at Visit 3.
1. Vital signs are measured and recorded.
2. The caregiver is queried regarding AEs, concomitant medications (including OTC, vitamins, and supplements), nondrug therapies, and any nonpharmacological interventions for the treatment of agitation.
3. The following assessments are completed (the CMAI and NPI-AA should be administered before the CGIS-Agitation and CGIC-Agitation):
   CMAI
   NPI-AA
   CGIS-Agitation
   CGIC-Agitation
4. Register study visit in IWRS and a blister card kit number are assigned to patient (2-week blister card).
5. Unused study drug is accounted for compliance, and a new blister card is dispensed (2-week blister card).
6. Patient's Diary Card is reviewed for compliance and returned to the patient.

### 7.1.5. Visit 4 (Day 29±3-Day Window)

Visit 4 (Day 29) should occur in the morning. The morning dose of study drug can be administered at home any time prior to the clinic visit; the time of dosing should be noted by the patient/caregiver.

The following procedures are performed at Visit 4:
1. Vital signs are measured and recorded.
2. The caregiver is queried regarding AEs, concomitant medications (including OTC, vitamins, and supplements), nondrug therapies, and any nonpharmacological interventions for the treatment of agitation.
3. The following assessments are completed (the CMAI and NPI-AA should be administered before the CGIS-Agitation and CGIC-Agitation):
   CMAI
   NPI-AA
   CGIS-Agitation
   CGIC-Agitation
4. Register study visit in IWRS and a blister card kit number are assigned to patient (2-week blister card).
5. Unused study drug is accounted for compliance, and a new blister card is dispensed (2-week blister card).
6. Patient's Diary Card is reviewed for compliance and returned to the patient.

### 7.1.6. Visit 5 (Day 43±3-Day Window)

Visit 5 (Day 43) should occur in the morning prior to the morning dose of study drug. The following procedures are performed at Visit 5.

Before Dosing:
1. A blood sample is collected for safety laboratory assessments and PK assessments.

The following procedures may be performed at any time:
1. Vital signs are measured and recorded.
2. The caregiver is queried regarding AEs, concomitant medications (including OTC, vitamins, and supplements), nondrug therapies, and any nonpharmacological interventions for the treatment of agitation.
3. A resting 12-lead ECG is performed (at any time during this visit).
4. The following assessments are completed (the CMAI and NPI should be administered before the CGIS-Agitation and CGIC-Agitation):
   CMAI
   NPI
   CGIS-Agitation
   CGIC-Agitation
5. Register study visit in IWRS and a blister card kit number are assigned to patient (2-week blister card).
6. Unused study drug is accounted for compliance, and a new blister card is dispensed (2-week blister card).
7. Patient's Diary Card is reviewed for compliance and returned to the patient.

### 7.1.7. Visit 6 (Day 57±3-Day Window)

Visit 6 (Day 57) should occur in the morning. The morning dose of study drug can be administered at home any time prior to the clinic visit; the time of dosing should be noted by the patient/caregiver.

The following procedures are performed at Visit 6:
1. Vital signs are measured and recorded.
2. The caregiver is queried regarding AEs, concomitant medications (including OTC, vitamins, and supplements), nondrug therapies, and any nonpharmacological interventions for the treatment of agitation.
3. The following assessments are completed (the CMAI and NPI-AA should be administered before the CGIS-Agitation and CGIC-Agitation):
   CMAI
   NPI-AA
   CGIS-Agitation
   CGIC-Agitation
4. Register study visit in IWRS and a blister card kit number are assigned to patient (2-week blister card).
5. Unused study drug is accounted for compliance, and a new blister card is dispensed (2-week blister card).
6. Patient's Diary Card is reviewed for compliance and returned to the patient.

### 7.1.8. Visit 7 (Day 71±3-Day Window)

Visit 7 (Day 71) should occur in the morning. The morning dose of study drug can be administered at home any time before the clinical visit; the time of dosing should be noted by the patient/caregiver.

The following procedures are performed at Visit 7:
1. Vital signs are measured and recorded.
2. The caregiver is queried regarding AEs, concomitant medications (including OTC, vitamins, and supplements), nondrug therapies, and any nonpharmacological interventions for the treatment of agitation.
3. A blood sample is collected for PK assessments (at any time during this visit).
4. The following assessments are completed (the CMAI and NPI should be administered before the CGIS-Agitation and CGIC-Agitation):
   CMAI
   NPI
   CGIS-Agitation
   CGIC-Agitation
5. Register study visit in IWRS and a blister card kit number are assigned to patient (2-week blister card).
6. Unused study drug is accounted for compliance, and a new blister card is dispensed (2-week blister card).
7. Patient's Diary Card is reviewed for compliance and returned to the patient.

### 7.1.9. Visit 8 (Day 85±3-Day Window)/Early Termination

Visit 8 (Day 85/ET) should occur in the morning. The morning dose of study drug can be administered at home any time prior to the clinic visit; the time of dosing should be noted by the patient/caregiver.

Patients who withdraw prior to study completion are required to complete study procedures as listed for Visit 8/ET within 48 hours of the last dose of study drug.

The following procedures are performed at Visit 8 (or ET):
1. Vital signs are measured and recorded.
2. Weight is measured and recorded.
3. Physical and neurological examination is performed.
4. The caregiver is queried regarding AEs, concomitant medications (including OTC, vitamins, and supplements), nondrug therapies, and any nonpharmacological interventions for the treatment of agitation.
5. A resting 12-lead ECG is performed (any time during this visit).
6. A blood and urine specimen is collected for safety laboratory assessments.
7. A urine pregnancy test is performed for women of childbearing potential only.
8. The following assessments are completed (the CMAI and NPI should be administered before the CGIS-Agitation and CGIC-Agitation):
   CMAI
   NPI
   CGIS-Agitation
   CGIC-Agitation
   EQ-5D-5L
   RUD-Lite
   MMSE
   ESS (only for patients with an MMSE score of ≥10 at Baseline)
   S-STS
9. Register study visit in IWRS.
10. Any unused study drug is returned and accounted for compliance.
11. Patient's Diary Card is reviewed for compliance.
12. Any previously reported and not yet resolved AE and any newly reported AE at the time of this visit are followed-up for up to 30 days after the last dose of study drug.

Caregivers and patients are instructed to return to the clinic for a Follow-up visit 30 days after the last dose of study drug.

### 7.1.10. Follow-Up Visit (30 Days Post Last Dose; +7-Day Window)

The Follow-up visit should occur 30 days (+7-day window) after the last dose of study drug.

The following procedures are performed:
1. The caregiver is queried regarding AEs, concomitant medications (including OTC, vitamins, and supplements), nondrug therapies, and any nonpharmacological interventions for the treatment of agitation.
2. The following assessments are completed:
   CMAI (completed first)
   CGIS-Agitation

### 8. Statistics

### 8.1. Analysis Populations

The following analysis population are defined for this study:
Enrolled: all patients enrolled in this study
Randomized: all patients who are randomized into this study
Safety: all patients who are randomized in this study and take at least one dose of study drug
Efficacy: all patients in the randomized population who take at least 1 dose of study drug, have a baseline CMAI total score, and at least 1 postbaseline evaluation for the CMAI total score.

In general, baseline of an efficacy endpoint is defined as the last observation of the endpoint before the patient is randomized.

The core dataset for all efficacy analyses is based on the ITT population, which is defined in the efficacy analysis set above. As is described below, in order to handle missing data and restrictions imposed by different types of analyses (e.g., change from baseline analysis), datasets derived from the ITT population are used for the efficacy analyses.

### 8.2. Efficacy Analysis

### 8.2.1. Primary Efficacy Endpoint Analysis

The primary efficacy endpoint is the change from baseline to the end of the efficacy period in the CMAI total score. The change from baseline to the end of the efficacy period in the CMAI total score is analyzed using a MMRM analysis with an unstructured variance covariance structure (UN). The model will include fixed class effect terms for treatment, study site, baseline concomitant antipsychotic use (yes/no), visit week, and an interaction term of treatment by visit week, and will include the interaction term of baseline values of the CMAI total score and NPI-AA score by visit week as a covariate. All scheduled visits after baseline during the double-blind treatment period are included in the model.

### 8.2.2. Key Secondary Analysis

The key secondary efficacy variable is the change from baseline to end of efficacy period in the CGI-S score, as related to agitation. It is analyzed by the same statistical methodology specified for the analysis of the primary efficacy variable.

### 8.2.3. Other Efficacy Endpoint Analysis

Other efficacy variables include the following:
Change from baseline to each study visit in efficacy period in CMAI Total score
Change from baseline to each study visit in efficacy period in CGIS-Agitation score
CGIC-Agitation score at each study visit in the efficacy period
Change from baseline to each study visit in efficacy period in NPI-AA score
Change from baseline to each study visit in efficacy period in NPI total score
CMAI Response Rate at each study visit in efficacy period, where response is defined as ≥30% reduction in CMAI Total Score from baseline
CMAI Response Rate at each study visit in efficacy period, where response is defined as ≥50% reduction in CMAI Total Score from baseline
Change from baseline to each study visit in efficacy period in the EQ-5D-5L total score
Change from baseline to each study visit in efficacy period in the RUD-Lite

Change from baseline is evaluated using the same MMRM model described in the primary analysis.

Change from baseline for the endpoints with one postbaseline assessment is evaluated using analysis of covariance (ANCOVA) with baseline value, study site and baseline concomitant antipsychotic use (yes/no) as a covariate and treatment as main factor.

The response variables are evaluated by the Cochran-Mantel-Haenszel (CMH) General Association Test controlling, in last-observation-carried-forward (LOCF) analyses, for study site and baseline concomitant antipsychotic use (yes/no). The OC analysis will not control for stratification factors.

The CGIC-Agitation score is evaluated by the Cochran-Mantel-Haenszel row mean score differ test (van Eltern), controlling for study site and baseline concomitant antipsychotic use (yes/no) in the LOCF analysis. The OC analysis will not control for stratification factors.

### 8.3. Safety Analysis

### 8.3.1. Electrocardiogram Data

Mean change from baseline is summarized by treatment group and by visit. Incidence of clinically relevant changes is calculated for ECG parameters and summarized by treatment group and by visit.

The analysis of QT and corrected QT interval (QTc) data from 3 consecutive complexes (representing 3 consecutive heart beats) is measured to determine average values. The following QT corrections are used:
QTcF is the length of the QT interval corrected for heart rate by the Fridericia's formula: QTcF=QT/(RR)0.33

Results are summarized by visit.

### Appendix 1. Prohibited Concomitant Medications

Patients who are currently taking or have taken any of the following types of medications within 2 weeks or 5 half-lives, whichever is longer, prior to the Baseline visit are not eligible for participation in the study. For psychotropic concomitant medication (in italics), please refer to Table 3 for information about the duration of washout prior to the Screening visit.

The list of prohibited concomitant medications includes, but is not limited, to the following:
A. Certain medications that may increase Q levels (exclusion does not include topical medications unless applied under occlusive dressing or other technique that is intended to increase systemic absorption):
   amiodarone
   antifungals (e.g., fluconazole, itraconazole, ketoconazole, posaconazole, voriconazole)
   carbonic anhydrase inhibitors (strong inhibitors are prohibited; topiramate is allowed)
   cimetidine
   delavirdine
   diltiazem
   macrolide antibiotics (e.g., clarithromycin, dirithromycin, erythromycin, roxithromycin, telithromycin)
   mexiletine
   nefazodone
   protease inhibitors (e.g., amprenavir, atazanavir, fosamprenavir, indinavir, ritonavir, saquinavir)
   ranolazine
   verapamil
B. Certain medications that may have increased plasma levels if co-administered with Q:
   atomoxetine
   tricyclic antidepressants (e.g., amitriptyline, amoxapine, clomipramine, desipramine, doxepin, imipramine, nortriptyline, protriptyline)
C. Medications that are related to Q:
   mefloquine
   quinidine
   quinine
D. Monoamine oxidase inhibitors (may increase the risk of serotonin syndrome)
E. Strong CYP3A4 inducers that may decrease DM or Q plasma levels:
   apalutamide
   carbamazepine
   dexamethasone
   enzalutamide
   fosphenytoin
   lumacaftor
   mitotane
   pentobarbital
   phenobarbital
   phenytoin
   primidone
   rifampicin
   rifamycin
   rifaximin
   St. John's wort
F. Certain medications that may be prescribed for the treatment of agitation or other indications:
   phenothiazines (e.g., chlorpromazine, fluphenazine, levomepromazine, methotrimeprazine, mesoridazine, phenocyazine, perphenazine, prochlorperazine, promazine, thioridazine, thiothixene, trifluoperazine, triflupromazine)
   typical antipsychotics (e.g., droperidol, haloperidol, loxapine, molindone, pimozide, zuclopenthixol)
G. Medications containing dextromethorphan (over-the-counter [OTC] and prescription)

### Example 2

A Phase 3, multicenter, randomized, double-blind, placebo-controlled, parallel-design study to assess the efficacy, safety, and tolerability of AVP-786 (deudextromethorphan hydrobromide [d6-DM]/quinidine sulfate [Q]) for the treatment of agitation in patients with dementia of the Alzheimer's type

### List of Abbreviations Used in Example 2

| Abbreviation | Definition |
|---|---|
| AA | Alzheimer’s Association |
| AE | Adverse event |
| AD | Alzheimer’s Disease |
| ADCS | Alzheimer’s Disease Cooperative Study |
| ADWG | Agitation Definition Work Group |
| ALT/SGPT | Alanine aminotransferase/serum glutamic-pyruvic |
| | transaminase |
| AST/SGOT | Aspartate aminotransferase/serum glutamic- |
| | oxaloacetic transaminase |
| BID | Twice daily |
| BP | Blood pressure |
| BUN | Blood urea nitrogen |
| CD-ROM | Compact disc read-only-memory |
| CFR | Code of Federal Regulations |
| Cit AD | Citalopram study for Agitation in Alzheimer’s |
| | disease |
| CGIC | Clinical Global Impression of Change |
| CGIS-Agitation | Clinical Global Impression of Severity of Illness |
| | scale for Agitation |
| CK | Creatine kinase |
| CMAI | Cohen-Mansfield Agitation Inventory |
| CNS | Central nervous system |
| CRO | Contract research organization |
| CSDD | Cornell Scale for Depression in Dementia |
| CYP | Cytochrome P450 |
| d6-DM | Deuterated (d6)-dextromethorphan hydrobromide |
| | (or free base form) |
| DM | Dextromethorphan hydrobromide (or free base form) |
| DMP | Data management plan |
| DSMB | Data and Safety Monitoring Board |
| DSM-IV-TR | Diagnostic and Statistical Manual of Mental |
| | Disorders |
| EC | Ethics Committee |
| ECDEU | Early Clinical Drug Evaluation Unit |
| ECG | Electrocardiogram |
| eCRF | Electronic case report form |
| EDC | Electronic data capture |
| EP | European Pharmacopeia |
| EQ-5D-5L | EuroQol 5-Dimension 5-Level |
| EQ VAS | EuroQol Visual Analogue Scale |
| ESS | Epworth Sleepiness Scale |
| EU | European Union |
| FDA | US Food and Drag Administration |
| GCP | Good Clinical Practice |
| GGT | Gamma-glutamyl transferase |
| GMP | Good Manufacturing Practice |
| HbA1c | Glycosylated hemoglobin |
| HR | Heart rate |
| ICF | Informed consent form |
| ICH | International Council for Harmonisation |
| IP | Investigational product |
| IPA | International Psychogeriatric Association |
| IRB | Institutional Review Board |
| IIT | Intent-to-Treat |
| IWRS | Interactive Web Response System |
| LDH | Lactate dehydrogenase |
| LOCF | Last observation carried forward |
| MAOI | Monoamine oxidase inhibitor |
| MedDRA | Medical Dictionary for Regulatory Activities |
| mITT | Modified Intent-to-Treat |
| MM | Medical Monitor |
| MMRM | Mixed effects model repeated measures |
| MMSE | Mini-Mental State Examination |
| MS | Multiple Sclerosis |
| NF | National Formulary |
| NIA | National Institute on Aging |
| NPI | Neuropsychiatric Inventory |
| NPI-NH | Neuropsychiatric Inventory-Nursing Home version |
| OTC | Over-the-counter |
| PBEF | Pregnancy and Breastfeeding Exposure Form |
| PGIC | Patient Global Impression of Change |
| PK | Pharmacokinetics |
| PR | The P-R interval from an ECG tracing |
| Q | Quinidine sulfate (or free base form) |
| QRS | The Q-R-S complex from an ECG tracing |
| QT | QT interval from an ECG tracing |
| QTe | QT interval corrected for heart rate |
| QTcF | QT interval corrected for heart rate using the |
| | Fridericia's formula |
| RBC | Red blood cell |
| SAE | Serious adverse event |
| SAP | Statistical analysis plan |
| SNRI | Serotonin-norepinephrine reuptake inhibitor |
| SOC | System organ class |
| SSRI | Selective serotonin reuptake inhibitor |
| S-STS | Sheehan Suicidality Tracking Scale |
| T3 | Triiodothyronine |
| T4 | Thyroxine |
| TEAE | Treatment-emergent adverse event |
| TSH | Thyroid-stimulating hormone |
| TUG | Timed Up and Go |
| UK | United Kingdom |
| US | United States |
| USP | United States Pharmacopoeia |
| WBC | White blood cell |

Title: A Phase 3, muiticenter, randomized, double-blind, placebo-controlled, parallel-design study to assess the efficacy, safety, and tolerability of AVP-786 (deudextromethorphan hydrobromide [db-DM]/quinidine sulfate [Q]) for the treatment of agitation in patients with dementia of the Alzheimer's type.

Study Objectives

The primary objective is to:
Evaluate the efficacy, safety, and tolerability of AVP-786 compared to placebo, for the treatment of agitation in patients with dementia of the Alzheimer's type.

The secondary objectives are to:
Evaluate the effects of AVP-786 compared to placebo on neuropsychiatric symptoms
Evaluate the effects of AVP-786 compared to placebo on global assessments of severity and improvement of agitation
Evaluate the effects of AVP-786 compared to placebo on quality of life.

Study Population

Condition/Disease: Patients with agitation secondary to dementia of the Alzheimer's type. The diagnosis of probable Alzheimer's disease will be based on the ‘2011 Diagnostic Guidelines for Alzheimer's Disease’ issued by the National Institute on Aging (NIA)-Alzheimer's Association (AA) workgroups Diagnosis of agitation will be based on the provisional consensus definition of agitation in patients with cognitive disorders developed by the International Psychogeriatric Association (IPA) Agitation Definition Work Group.

Key Inclusion Criteria: Patients with clinically significant, moderate/severe agitation at the time of screening and for at least 2 weeks prior to randomization, that interferes with daily routine and for which a prescription medication is indicated in the opinion of the investigator. A Clinical Global Impression of Severity of Illness scale for Agitation (CGIS-Agitation) score of ≥4 (moderately ill) at screening and baseline is required for study participation.

Key Exclusion Criteria: Patients with dementia predominantly of the non-Alzheimer's type (e.g., vascular dementia, frontotemporal dementia, Parkinson's disease, substance-induced dementia) and patients with symptoms of agitation that are not secondary to Alzheimer's disease (e.g., secondary to pain, other psychiatric disorder or delirium) are not eligible.

A complete list of inclusion/exclusion criteria is presented elsewhere in this Example.

Study Design

Structure: This is a Phase 3, multicenter, randomized, double-blind, placebo-controlled, parallel-design study.

Duration: Patients will be enrolled in the study for approximately 16 weeks; with up to 4-week screening period and 12-week treatment period.

End of Trial: The end of trial is defined as the “Last Patient Last Visit”; which is the date on which the last patient has his or her last visit or assessment (either for therapeutic or follow-up purposes including a follow-up phone call).

Study Treatment: The investigational product is AVP-786 (deudextromethorphan hydrobromide [d6-DM]/quinidine sulfate [Q]). Three doses of AVP-786 will be used in the study, d6-DM 18 mg/Q 4.9 mg, d6-DM 28 mg/Q 4.9 mg, and d6-DM 42.63 mg/Q 4.9 mg, hereafter referred to as AVP-786-18/4.9, AVP-786-28/4.9, and AVP-786-42.63/4.9, respectively.

Control: Placebo capsules of identical appearance to study medication will be used as control.

Randomization/Stratification: Eligible patients will be randomized into the study to either AVP-786-28/4.9, AVP-786-42.63/4.9 or placebo group. The randomization will be stratified by the concomitant use of antipsychotic medications (yes vs. no) and study site.

Dose Regimen: Eligible patients will be randomly assigned at the Baseline visit to receive AVP-786 or matching placebo capsules. Study medication will be administered orally twice daily (BID, 1 capsule in the morning and 1 capsule in the evening approximately 12 hours apart) throughout the study.
Patients randomized to the AVP-786-28/4.9 group will start with AVP-786-18/4.9 once a day in the morning and placebo in the evening for the first 7 days of the study. From Day 8, patients will receive AVP-786-18/4.9 BID for 14 days. From Day 22, patients will receive AVP-786-28/4.9 BID for the remaining 9 weeks of the study. If deemed necessary by the investigator, a one-time downward dose adjustment to AVP-786-18/4.9 will be allowed after Visit 3 up to and including Visit 4 (i.e., Day 23 to Day 43), and the patient will remain on the lower dose of study medication for the remaining study duration. Patients requiring a dose-adjustment between study visits will need to have an unscheduled visit to perform safety assessments.
Patients randomized to the AVP-786-42.63/4.9 group will start with AVP-786-28/4.9 once a day in the morning and placebo in the evening for the first 7 days of the study. From Day 8, patients will receive AVP-786-28/4.9 BID for 14 days. From Day 22, patients will receive AVP-786-42.63/4.9 BID for the remaining 9 weeks of the study. If deemed necessary by the investigator, a one-time downward dose adjustment to AVP-786-28/4.9 will be allowed after Visit 3 up to and including Visit 4 (i.e., Day 23 to Day 43), and the patient will remain on the lower dose of study medication for the remaining study duration. Patients requiring a dose-adjustment between study visits will need to have an unscheduled visit to perform safety assessments.
Patients randomized to receive placebo will be dosed with placebo BID for the 12-week treatment period.

Assessments and Visits

Patients will attend clinic visits at Screening, Baseline (Day 1), and on Visit 2 (Day 8), Visit 2.1 (Day 15), Visit 3 (Day 22), Visit 4 (Day 43), Visit 5 (Day 64), and Visit 6 (Day 85). Patients who terminate early will receive daily phone calls for 5 consecutive days following the early termination (ET) visit to query on their overall well-being and will be asked to return for an in-clinic Follow-up visit 30 days after last dose of study medication for selected safety and efficacy assessments. Safety follow-up phone calls will be made on Day 29 and Day 71. Study procedures will be performed at each visit as outlined in the Schedule of Evaluations and Visits (Table 4).

Response Measures

Efficacy

Primary Efficacy Measure: Primary efficacy will be assessed using the Cohen-Mansfield Agitation Inventory (CMAI).

Secondary Efficacy Measure: Clinical Global Impression of Severity of Illness for Agitation (CGIS-Agitation)

Other Efficacy Measures: Agitation/Aggression domain of the NPI, NPI-Agitation/Aggression domain Caregiver Distress score, NPI—Aberrant Motor Behavior domain, NPI—Irritability/Lability domain, Patient Global Impression of Change (PGIC-rated by caregiver), total NPI, and EuroQol 5-Dimension 5-Level (EQ-5D-5L).

For consistency of rating, assessments should be performed by the same rater throughout the study. The following scales MUST be administered by the same rater at each visit. CMAI, NPI, and CGIS-Agitation.

Pharmacokinetics

Plasma concentrations of d6-DM, its metabolites, and Q will be measured.

Safety and Tolerability

Safety and tolerability of AVP-786 will be assessed by reported adverse events (AEs), physical and neurological examinations, vital signs, clinical laboratory assessments, resting 12-lead electrocardiograms (ECGs), Sheehan Suicidality Tracking Scale (S-STS), Mini Mental State Examination (MMSE), and the Epworth Sleepiness Scale (ESS).

Pregnancy tests will be conducted for females of childbearing potential.

General Statistical Methods and Types of Analyses

Analysis Populations

Three analysis populations will be used; modified intent-to-treat (mITT), intent-to-treat (ITT), and safety. The mITT population includes all patients randomized in the study who had at least one post-baseline efficacy assessment, and will be used for all analyses of efficacy. Patients in the mITT population will be included in the treatment group to which they were randomized regardless of treatment received. The ITT population includes all randomized patients in the study, and will be used for exploratory efficacy analyses. The safety population includes all patients who received study treatment, and will be used for all analyses of safety. Patients will be included in the treatment group based on the actual treatment received.

Efficacy Analyses

The primary efficacy endpoint is the change from Baseline to Week 12 (Day 85) in the CMAI total score. The primary treatment comparisons (AVP-786-42.63/4.9 vs. placebo and AVP-786-28/4.9 vs. placebo) will be performed by using a linear mixed effects model repeated measures (MMRM) with fixed effects for treatment, visit, treatment-by-visit interaction, baseline-by-visit interaction, and baseline covariates which include baseline value and other factors as appropriate. An unstructured covariance model will be used. In addition, the primary endpoint will also be analyzed with missing data imputed by other statistical methods, such as multiple imputation. Details will be pre-specified in the statistical analysis plan (SAP).

Secondary and other efficacy endpoints include change from Baseline to Week 12 (Day 85) for the following efficacy measures: CGIS-Agitation, NPI-Agitation/Aggression domain score and Caregiver Distress score, NPI—Aberrant Motor Behavior domain score, NPI—Irritability/Lability domain score, PGIC, total NPI, and EQ-5D-5L.

TABLE 4
Schedule of Evaluations and Visits

| | Visit: | | | | | | | | | | |
|---|---|---|---|---|---|---|---|---|---|---|---|
| | | | Visit | Visit | Visit | Phone | Visit | Visit | Phone | Visit 6^{1,4}/ | Follow-up |
| | Screening¹ | Baseline | 2¹ | 2.1¹ | 3^{1,17} | Call^{1,2} | 4^{1,17} | 5¹ | Call^{1,2} | ET³ | Visit³ |
| | Study Day: | | | | | | | | | | |
| | Day −28 | | | | | | | | | | 30-days |
| | to −1 | Day 1 | Day 8 | Day 15 | Day 22 | Day 29 | Day 43 | Day 64 | Day 71 | Day 85 | Post-dose |
| | End of Study Week: | | | | | | | | | | |
| | Week −4 | | | | | | | | | | |
| Procedure | to −1 | | Week 1 | Week 2 | Week 3 | Week 4 | Week 6 | Week 9 | Week 10 | Week 12 | |
| Sign informed consent | X | | | | | | | | | | |
| forms | | | | | | | | | | | |
| Medical history | X | | | | | | | | | | |
| Review of eligibility | X⁵ | X | | | | | | | | | |
| Randomization | | X | | | | | | | | | |
| Physical and neurological | X | | | | | | | | | X | |
| examination | | | | | | | | | | | |
| Vital signs, height, and | X | X⁶ | X | X | X | | X | X | | X⁶ | |
| weight | | | | | | | | | | | |
| CGIS-Agitation | X | X | | | | | X | | | X | |
| Risk assessment for falls | X | | | | | | | | | | |
| (worksheet and TUG test) | | | | | | | | | | | |
| ECG⁸ | X⁷ | X | | X | X | | X | X | | X | |
| AEs | X | X | X | X | X | X | X | X | X | X | X |
| Prior and concomitant: | X | X | X | X | X | X | X | X | X | X | X |
| medications, nondrug | | | | | | | | | | | |
| therapies, and | | | | | | | | | | | |
| nonpharmacological | | | | | | | | | | | |
| interventions for agitation | | | | | | | | | | | |
| MMSE | X | X | | | | | | | | X | |
| CMAI | X | X | X | X | X | | X | X | | X | X |
| NPI | X⁹ | X | X⁹ | X⁹ | X | | X | X | | X | |
| CSDD | X | | | | | | | | | | |
| EQ-5D-5L¹⁰ | | X | | | | | X | | | X | |
| PGIC¹¹ | | | | | | | X | | | X | |
| ESS¹² | | X | | | | | X | | | X | |
| S-STS | X | X | X | X | X | | X | X | | X | X |
| Administer morning dose of | | X | | | | | | | | | |
| study medication in clinic¹³ | | | | | | | | | | | |
| Chemistry, hematology, and | X¹⁴ | | | | X | | X | X | | X¹⁴ | |
| urinalysis | | | | | | | | | | | |
| Urine pregnancy test¹⁵ | X | X | | | | | X | | | X | |
| PK blood sample¹⁶ | | X | | | | | X | X | | X | |
| CYP2D6 blood sample | | X | | | | | | | | | |
| Dispense study drug and | | X | | | X | | X | X | | | |
| diary card^{17, 18} | | | | | | | | | | | |
| Review and return unused | | | X | X | X | | X | X | | X | |
| study medication and | | | | | | | | | | | |
| diary card | | | | | | | | | | | |

AE = adverse event;
CGIS-Agitation = Clinical Global Impression of Severity of Illness scale for Agitation;
CMAI = Cohen-Mansfield Agitation Inventory;
CSDD = The Cornell Scale for Depression in Dementia;
ECG = electrocardiogram;
EQ-5D-5L = EuroQol 5-Dimension 5-Level;
ESS = Epworth Sleepiness Scale;
ET = early termination;
MMSE = Mini-Mental State Examination;
NPI = Neuropsychiatric Inventory;
PGIC = Patient Global Impression of Change rated by the caregiver; Pharmacokinetics;
S-STS = Sheehan Suicidality Tracking Scale;
TUG = Timed Up and Go
Note:
Whenever possible, each patient and caregiver should have the rating scales administered by the same raters throughout the study, for consistency of ratings. The following scales MUST be administered by the same rater at each visit: CMAI, NPI, and CGIS-Agitation.
¹Study visits have a ±3-day window except Screening (Day −28 to Day −1), Visit 2 (Day 8), and phone calls. Screening (Day −28 to Day −1), Visit 2 (Day 8), and phone calls (excludes follow-up phone calls for ET patients) have a +3-day window. The screening period may be extended after discussion with and approval by the Medical Monitor (MM).
²Phone call should be made to patient/caregiver to collect adverse events and query on concomitant medication usage.
³Early termination visit for patients who withdraw prior so study completion. Patients who terminate early from the study will receive daily phone calls for 5 consecutive days following the ET visit to query on their overall well-being and an in-clinic Follow-up visit 30 days after last dose of study medication for selected safety and efficacy assessments.
⁴Patients will receive a safety follow-up phone call 30 days after the last dose of study medication. In some regions patients may be eligible to enter a long-term extension study; for these patients, the safety follow-up phone call 30 days after last dose of study medication will not occur.
⁵For patients deemed eligible by the investigator, a protocol eligibility form will be completed and submitted to the MM.
⁶Height should be measured only at the Baseline Visit (Day 1). Weight should be measured only at the Baseline Visit (Day 1) and Visit 6 (Day 85).
⁷ECG should be performed in triplicate at the Screening Visit (Day −28 to Day −1).
⁸ECG to be performed pre-dose and at least 1 hour post-dose at Baseline Visit (Day 1). ECGs should be collected at any time during the other visits.
⁹Only the Agitation/Aggression domain of the NPI should be performed at the Screening Visit (Day −28 to Day −1), Visit 2 (Day 8), and Visit 2.1 (Day 15).
¹⁰The proxy version is to be rated by the caregiver. The non-proxy version is to be rated only by patients with an MMSE score of ≥10 at baseline.
¹¹PGIC is to be rated by the caregiver.
¹²ESS is to be rated only by patients with an MMSE score of ≥10 at the Baseline Visit (Day 1).
¹³The morning dose of study medication should be administered in the clinic at the Baseline Visit (Day 1).
¹⁴Thyroid function tests (TSH, and reflex T3 and T4 if TSH is abnormal) should be performed at the Screening Visit (Day −28 to Day −1). Glycosylated hemoglobin (HbA1c) test should be performed at the Screening Visit (Day −28 to Day −1) and Visit 6 (Day 85).
¹⁵Urine pregnancy test to be performed for patients of childbearing potential only.
¹⁶The PK blood sample should be collected 1 to 4 hours post-dose at the Baseline Visit (Day 1). PK samples should be collected at any time during Visit 4 (Day 43), Visit 5 (Day 64), and Visit 6 (Day 85); patients/caregivers should record time of last 2 doses prior to the clinic visit.
¹⁷Patients/caregivers should record the time of last 2 doses prior to the clinic visit. The blister card and diary card should be returned to the patient/caregiver after reviewing for compliance.
¹⁸ A one-time downward dose adjustment is allowed after Visit 3 (Day 22) up to and including Visit 4 (Day 43). Patient will need to return to the clinic for an unscheduled visit for safety assessments.

### 1. Study Design

This is a Phase 3, multicenter, randomized, double-blind, placebo-controlled, parallel-design study of 12-week treatment duration.

There will be 8 scheduled clinic visits including a screening visit, and 2 safety follow-up phone calls in this study. Patients will attend clinic visits at Screening, Baseline (Day 1), and on Visit 2 (Day 8), Visit 2.1 (Day 15), Visit 3 (Day 22), Visit 4 (Day 43), Visit 5 (Day 64), and Visit 6 (Day 85). Safety follow-up phone calls will be made on Day 29 (Week 4) and 71 (Week 10). Study procedures will be performed at each visit as outlined in the Schedule of Evaluations and Visits (Table 4).

Eligible patients will be randomly assigned at the Baseline visit to receive AVP-786 or matching placebo. Study medication will be administered orally twice daily from Baseline (Day 1) through Visit 6 (Day 85). Patients (or caregivers) will self-administer study medication on all study days except on applicable clinic-visit days when patients will be administered their morning dose of study medication at the clinic in the presence of site personnel, regardless of the time of day. Screening will occur within 4 weeks prior to randomization.

Following screening procedures for assessment of inclusion and exclusion criteria, eligible patients will be randomized into the study to either the AVP-786-28/4.9, AVP-786-42.63/4.9, or placebo group. The randomization will be stratified by the concomitant use of antipsychotic medications (yes vs. no) and study site. Study medication (active or placebo) will be administered orally BID (1 capsule in the morning and 1 capsule in the evening approximately 12 hours apart) throughout the treatment period.

Patients randomized to the AVP-786-28/4.9 group will start with AVP-786-18/4.9 once a day in the morning and placebo in the evening for the first 7 days of the study. From Visit 2 (Day 8), patients will receive AVP-786-18/4.9 BID for 14 days. From Visit 3 (Day 22), patients will receive AVP-786-28/4.9 BID for the remaining 9 weeks of the study. If deemed necessary by the investigator, a one-time downward dose adjustment to AVP-786-18/4.9 will be allowed after Visit 3 up to and including Visit 4 (i.e., Day 23 to Day 43), and the patient will remain on the lower dose of study medication for the remaining study duration. Patients requiring a dose-adjustment between study visits will need to have an unscheduled visit to perform safety assessments.

Patients randomized to the AVP-786-42.63/4.9 group will start with AVP-786-28/4.9 once a day in the morning and placebo in the evening for the first 7 days of the study. From Visit 2 (Day 8), patients will receive AVP-786-28/4.9 BID for 14 days. From Visit 3 (Day 22), patients will receive AVP-786-42.63/4.9 BID for the remaining 9 weeks of the study. If deemed necessary by the investigator, a one-time downward dose adjustment to AVP-786-28/4.9 will be allowed after Visit 3 up to and including Visit 4 (i.e., Day 23 to Day 43), and the patient will remain on the lower dose of study medication for the remaining study duration. Patients requiring a dose-adjustment between study visits will need to have an unscheduled visit to perform safety assessments.

Patients randomized to receive placebo will be dosed with placebo BID for the 12-week treatment period.

### 2. Study Population

Patients enrolled in this study must have a diagnosis of probable AD and must present with clinically meaningful, moderate/severe agitation secondary to AD.

The diagnosis of probable AD will be based on the ‘2011 Diagnostic Guidelines for Alzheimer's Disease’ issued by the National Institute on Aging (NIA)-Alzheimer's Association (AA) workgroups. These new criteria were developed based on the review of the NINCDS-ADRDA criteria. Neither AD nor agitation should be explainable by delirium, substance use and/or major psychiatric disorders.

The provisional consensus definition of agitation in patients with cognitive disorders developed by the Agitation Definition Work Group (ADWG) from the International Psychogeriatric Association (IPA) will be used for selecting study patients. This proposed definition is limited to patients with cognitive impairment and requires: (a) evidence of emotional distress; (b) one of 3 observable types of behaviors-excessive motor activity, verbal aggression, or physical aggression; (c) that the behavior causes excess disability; and (d) that the behaviors cannot be solely attributable to a suboptimal care environment or other disorder such as a psychiatric illness, a medical illness, or effects of a substance.

Eligible patients must have agitation (persistent or frequently recurrent) at the time of study screening and for at least 2 weeks prior to randomization and the agitation symptoms must be severe enough such that they interfere with daily routine and cause distress to the patient and caregiver for which a prescription medication is deemed indicated, in the opinion of the treating physician.

Agitation will further be assessed using the CGIS-Agitation scale (0-7). A score of ≥4 (moderately ill) at screening and baseline are required for study participation.

Eligible patients are to have otherwise acceptable and stable general health as required by the study protocol, and documented by medical history, physical examination, electrocardiogram (ECG), and clinical laboratory examinations.

Eligible patients must have a caregiver who is able and willing to comply with all required study procedures, ensuring that the patient attends all study visits and takes the study medication as instructed. Caregivers will also be instructed to keep a study diary, to report any changes in patient's status, including adverse events, standard of care setting (e.g., becoming a resident in an assisted living facility), and to provide their impression and assessment regarding the investigational treatment. In order to qualify as a reliable informant (i.e., caregiver) capable of assessing changes in the patient's condition during this study, the individual must spend a minimum of 2 hours per day for 4 days per week with the study patient.

### 2.1. Inclusion Criteria

1. Males and females 50 to 90 years of age inclusive, at the time of informed consent.
2. Diagnosis of probable AD according to the 2011 NIA-AA working groups criteria. Either out-patients or residents of an assisted-living facility or a skilled nursing home.
3. The patient has clinically significant, moderate/severe agitation at the time of screening and for at least 2 weeks prior to Baseline, that interferes with daily routine and for which a prescription medication is indicated, in the opinion of the investigator.
4. The diagnosis of agitation must meet the IPA provisional definition of agitation.
5. CGIS-Agitation score is ≥4 (moderately ill) at Screening and Baseline.
6. MMSE score between 6 and 26 (inclusive) at Screening and Baseline.
7. The patient has stable cardiac, pulmonary, hepatic, and renal function.
8. The patient has stable cardiac, pulmonary, hepatic, and renal function.
9. The patient has an ECG (obtained within the past month prior to Baseline and evaluated by a central ECG reader) with no clinically significant findings.
10. Patients of childbearing potential who are sexually active must use an effective method of birth control for at least 1 month prior to Baseline, during participation in the study, and for at least 30 days after the last dose of study drug. The following should be taken into consideration:
   Patients of childbearing potential must use 2 of the following precautions in order to minimize the risk of failure of 1 method of birth control: vasectomy, tubal ligation vaginal diaphragm, intrauterine device, birth control pills, birth control depot injection, birth control implant, or condom with spermicide or sponge with spermicide. Periodic abstinence (e.g., calendar, ovulation, symptothermal, post-ovulation methods), declaration of abstinence for the duration of exposure to study drug, or withdrawal are not acceptable methods of contraception.
   Patients who are sterile (i.e., had an oophorectomy and/or hysterectomy), postmenopausal (defined as 12 consecutive months with no menses without an alternative medical cause) or practice true abstinence (when this method is in line with the preferred and usual lifestyle of the patient) are exempt from this requirement.
   Patients who are lactating, pregnant or plan to become pregnant are excluded.
11. Use of medication for the treatment of AD (e.g., donepezil, rivastigmine, galantamine, memantine) is allowed provided the dose has been stable for at least 3 months prior to Baseline.
12. Concomitant use of antidepressants such as selective serotonin reuptake inhibitors (SSRIs; e.g., fluoxetine, sertraline, citalopram), serotonin-norepinephrine reuptake inhibitors (SNRIs; e.g., venlafaxine, desvenlafaxine, duloxetine) are allowed, provided the dose has been stable for at least 3 months prior to the Screening Visit and is within the Package Insert guidance for that medication. Paroxetine, a CYP2D6 substrate, is allowed provided the dose does not exceed 10 mg/day.
13. Concomitant use of hypnotics at bedtime (e.g., eszopiclone, zolpidem, zaleplon, trazodone [up to 50 mg/day]) for the nighttime treatment of insomnia is allowed, provided the dose has been stable for at least 1 month prior to Baseline and remains stable throughout the study.
14. Patients currently taking allowed medications for the treatment of agitation secondary to AD (e.g., atypical antipsychotics, buspirone) are eligible provided they have been on a stable dose for at least 2 weeks prior to Screening and at least 1 month stability prior to Baseline. Patients currently on a stable dose(s) of allowed antidepressant medication(s) for at least 3 months prior to the Screening Visit are eligible.
15. Patient must not show current and significant symptoms of a depressive disorder and must have a score <10 in The Cornell Scale for Depression in Dementia (CSDD) at Screening.
16. Patient must have no history or current clinical symptoms of schizophrenia, schizoaffective disorder, or bipolar disorder, as defined in the Diagnostic and Statistical Manual of Mental Disorders, 4^{th} Edition, Text Revision (DSM-IV-TR).

### 2.2. Exclusion Criteria

1. Caregiver is unwilling or unable, in the opinion of the investigator, to comply with study instructions.
2. Patient has dementia predominantly of non-Alzheimer's type (e.g., vascular dementia, frontotemporal dementia, Parkinson's disease, substance-induced dementia).
3. Patients with symptoms of agitation that are not secondary to AD (e.g., secondary to pain, other psychiatric disorder, or delirium).
4. Patients with myasthenia gravis (contraindication for quinidine).
5. Patients with any personal history of complete heart block, QTc prolongation, or torsades de pointes.
   Screening and Baseline QT interval corrected for heart rate using the Fridericia's formula (QTcF) of >450 msec for males and >470 msec for females unless due to ventricular pacing (Screening ECG will be based on central review. Baseline pre-dose ECG will be based on the machine read and investigator's evaluation. If the Baseline pre-dose ECG QTcF result from the machine read is exclusionary, do not dose the patient and please contact the Medical Monitor)
   Presence of premature ventricular contractions (PVCs) as evaluated by a central reader and deemed clinically significant by the investigator
6. Patients with any family history of congenital QT interval prolongation syndrome.
7. Patients with known hypersensitivity to DM, Q, opiate drugs (codeine, etc.), or any other ingredient of the study medication.
8. Exclusion Criterion was removed in Protocol Amendment 4.
9. Patients who have ever received DM co-administered with Q.
10. Patients who have been taking disallowed concomitant medications within 2 weeks or 5 half-lives, whichever is longer, prior to Baseline.
11. Patients with co-existent clinically significant or unstable systemic diseases that could confound the interpretation of the safety results of the study (e.g., malignancy [except skin basal-cell carcinoma], poorly controlled diabetes, poorly controlled hypertension, unstable pulmonary, renal or hepatic disease, unstable ischemic cardiac disease, dilated cardiomyopathy, or unstable valvular heart disease). Certain other non-metastatic cancer may be allowed. Each case to be evaluated individually with the Medical Monitor (MM).
12. Patients who are currently participating in, or who have participated in other interventional (drug or device) clinical study, or for patients in the United States that are found to be a “Virtually Certain” match in Clinical Trial Subject Database (CTS database) with a patient who has participated in another interventional drug or device study within 30 days of Baseline.
13. Patients with history of postural syncope, or any history of unexplained syncope (evaluated on a case by case basis) within 12 months of Baseline.
14. Patients with a history of substance and/or alcohol abuse within the past 1 year.
15. Patients determined to have a high imminent risk of falls during the study based on a clinical evaluation by the investigator.
16. Patients with evidence of serious risk of suicide at Screening or Baseline based on the Sheehan Suicidality Tracking Scale (S-STS), i.e., a score of 3 or 4 on any one question 2 through 6 or 11 or a score of 2 or higher on any one questions 1a, 7 through 10, or 12, or who, in the opinion of the investigator, present a serious risk of suicide.
17. Patients who, in the opinion of the Investigator, Medical Monitor, or sponsor, should not participate in the study.

### 2.3. Patient Withdrawal from the Study

Patients and caregivers will be advised verbally and in the written ICF that they have the right to withdraw from the study at any time without prejudice or loss of benefits to which they are otherwise entitled. The investigator or sponsor may discontinue a patient from the study in the event of an intercurrent illness, adverse event, other reasons concerning the health or well-being of the patient, or in the case of lack of cooperation, non-compliance, protocol violation, or other administrative reasons. If a patient does not return for a scheduled visit, every effort should be made to contact the patient. Regardless of the circumstance, every effort should be made to document patient outcome, if possible. The investigator should inquire about the reason for withdrawal, request the caregiver return all unused investigational product (IP), and follow-up with the patient regarding any unresolved adverse events.

In addition, patients who present with a persistent QTc interval (QTcF)>500 msec (unless due to ventricular pacing) or a persistent QTcF interval change from the pre-dose Baseline ECG of >60 msec, that is confirmed by the central ECG reader, at any time after randomization, will be withdrawn from the study after consultation with the Medical Monitor. The QTcF values will be assessed for clinical significance and recorded.

Patients who terminate early will be asked to return to the clinic to complete the Visit 6 (Day 85) assessments and an in-clinic Follow-up visit, 30 days after last dose of study medication for selected safety and efficacy assessments. In addition, daily phone calls for 5 consecutive days following ET visit will be made for these patients to assess their overall well-being.

If the patient withdraws from the study, and consent is withdrawn by the caregiver and/or patient's representative for disclosure of future information, no further evaluations should be performed, and no additional data should be collected. The sponsor may retain and continue to use any data collected before such withdrawal of consent. Patients who withdraw from the study will not be replaced.

### 3. Study Treatments

### 3.1. Treatments Administered

### 3.1.1. Description of Study Medications

Clinical study medication will be provided as hard, printed, opaque, blue, gelatin capsules (size 3). Each capsule of the study medication contains 1 of the following:
42.63 mg of d6-DM and 4.9 mg of Q (USP, EP): AVP-786-42.63/4.9
28 mg of d6-DM and 4.9 mg of Q (USP, EP): AVP-786-28/4.9
18 mg of d6-DM and 4.9 mg of Q (USP, EP): AVP-786-18/4.9
AVP-786 placebo

Drug supplies will be provided to the site in double-blind, individual, pre-labeled blister cards.

### 3.1.2. Composition of AVP-786

The qualitative compositions of the 3 doses of the IP and the placebo are listed in Table 5.

TABLE 5
Composition of Investigational Product

| | AVP-786- | AVP-786- | AVP-786- | AVP-786 |
|---|---|---|---|---|
| Ingredient (amounts in mg) | 42.63/4.9 | 28/4.9 | 18/4.9 | Placebo |
| Deudextromethorphan hydrobromide (*) | 42.63 (33) | 28.00 (21.67) | 18.00 (13.93) | 0 |
| Quinidine sulfate USP, EP (*) | 4.9 (4.26) | 4.9 (4.26) | 4.9 (4.26) | 0 |

EP = European Pharmacopoeia;
USP = United States Pharmacopoeia;
NF = National Formulary
(*) Free base equivalent indicated in parenthesis

### 3.2. Methods of Assigning Patients to Treatment Groups

### 3.2.1. Randomization

Eligible patients will be randomized to AVP-786-28/4.9, AVP-786-42.63/4.9, or placebo, respectively, at Baseline (Day 1). The randomization will be stratified by the concomitant use of antipsychotic medications (yes vs. no) and study site.

### 3.3. Concomitant Medications and Nondrug Therapies

Patients may not take any of the disallowed medications listed in Appendix 2 during the study or 2 weeks or 5 half-lives, whichever is longer, prior to the start of dosing on Day 1. At each visit, caregivers will be queried as to whether or not the patient has taken any concomitant medications and, if so, the investigator will record the medications taken and the reasons for their use.

AVP-786 contains quinidine which is a P-glycoprotein inhibitor. Concomitant administration of quinidine with digoxin, a P-glycoprotein substrate, results in serum digoxin levels that may be as much as doubled. Plasma digoxin concentrations should be closely monitored in patients taking digoxin concomitantly and dose reduced, as necessary.

In cases of prodrugs whose actions are mediated by the CYP2D6-produced metabolites (for example, codeine and hydrocodone, whose analgesic and antitussive effects appear to be mediated by morphine and hydromorphone, respectively), it may not be possible to achieve the desired clinical benefits in the presence of AVP-786 due to quinidine-mediated inhibition of CYP2D6. Alternative treatment should be considered.

### 3.3.1. Allowed Concomitant Medications

Drugs for the treatment of AD (e.g., donepezil, rivastigmine, galantamine, memantine) are allowed when administered at stable dose for at least 3 months prior to Baseline; the dose of these drugs should remain unchanged throughout the study. If dose adjustment is necessary, the new dose and the reason for the change should be recorded.

The use of drugs for the treatment of agitation secondary to AD (e.g., atypical antipsychotics, buspirone) is allowed, provided the patient has been on a stable dose for at least 2 weeks prior to screening and at least 1 month prior to Baseline and throughout the study. Patients on a stable dose(s) of allowed antidepressant medication(s) for at least 3 months prior to the Screening Visit are eligible.

Concomitant use of antidepressants such as SSRIs (e.g., fluoxetine, sertraline, citalopram), SNRIs (e.g., venlafaxine, desvenlafaxine, duloxetine) are allowed, provided the dose has been stable for at least 3 months prior to the Screening Visit and is within the Package Insert guidance for that medication. Paroxetine, a CYP2D6 substrate, is allowed provided the dose does not exceed 10 mg/day. SSRIs, SNRIs, and paroxetine must remain stable throughout the study unless a dose reduction is deemed necessary for management of an adverse event.

Patients taking SSRIs or SNRIs concomitantly should be monitored for serotonin syndrome which includes altered mental status, hypertension, restlessness, myoclonus, hyperthermia, hyperreflexia, diaphoresis, shivering, and tremor.

Concomitant use of hypnotics at bedtime (e.g., eszopiclone, zolpidem, zaleplon, trazodone [up to 50 mg/day]) for the nighttime treatment of insomnia is allowed, provided the dose has been stable for at least 1 month prior to Baseline and remains stable throughout the study.

### 3.3.2. Rescue Medication for the Symptoms of Agitation

No rescue medications are allowed.

### 3.3.3. Prohibited Concomitant Medications

A list of examples of prohibited medications is provided in Appendix 2.

Monoamine oxidase inhibitors (MAOI) are prohibited throughout the study. Patients should allow at least 14 days after stopping study medication before starting an MAOI.

### 3.3.4. Nondrug Therapies

Information on any prior and concomitant nondrug therapies will be recorded.

### 3.3.5. Nonpharmacological Interventions for the Treatment of Agitation

Information on any nonpharmacological interventions for the treatment of agitation that were used prior to enrollment or used concomitantly during the study will be recorded.

### 4. Study Assessments and Procedures

Whenever possible, each patient and caregiver should have the rating scales administered by the same raters throughout the study, for consistency of ratings. The following scales MUST be administered by the same rater at each visit: CMAI, NPI, and CGIS-Agitation.

### 4.1. Screening

### 4.1.1. Cornell Scale for Depression in Dementia (CSDD)

The CSDD was specifically developed to assess signs and symptoms of major depression in patients with dementia. Because some of these patients may give unreliable reports, the CSDD uses a comprehensive interviewing approach that derives information from the patient and the caregiver. Information is elicited through two semi-structured interviews; an interview with a caregiver and an interview with the patient. The interviews focus on depressive symptoms and signs occurring during the week preceding the assessment. The CSDD takes approximately 20 minutes to administer.

Each item is rated for severity on a scale of 0-2 (0=absent, 1=mild or intermittent, 2=severe). The item scores are added. Scores above 10 indicate a probable major depression, scores above 18 indicate a definite major depression, and scores below 6 as a rule are associated with absence of significant depressive symptoms.

The CSDD will be assessed at Screening (Day −28 to Day −1) only. Patients with a score of <10 will be included in the study.

### 4.1.2. Timed Up and go (TUG) Test

The TUG test measures the time (in seconds) taken for an individual to stand up from a standard armchair, walk 3 meters, turn, walk back to the chair and sit down. It is a commonly used scale for measuring functional mobility and risk of falls.

The TUG test will be performed at Screening (Day −28 to Day −1) only.

### 4.2. Efficacy

### 4.2.1. Cohen-Mansfield Agitation Inventory (CMAI)

The CMAI will be used as the primary efficacy measure in this study. The CMAI is used to assess the frequency of manifestations of agitated behaviors in elderly persons. It consists of 29 agitated behaviors that are further categorized into distinct agitation syndromes, also known as CMAI factors of agitation. These distinct agitation syndromes include: aggressive behavior, physically non-aggressive behavior, and verbally agitated behavior. Scores for the 3 dimensions Factor 1, Factor 2, and Factor 3 will be derived based on the factor structure described by Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998 and elsewhere herein. Each of the 29 items is rated on a 7-point scale of frequency (1=never, 2=less than once a week but still occurring, 3=once or twice a week, 4=several times a week, 5=once or twice a day, 6=several times a day, 7=several times an hour). The ratings are based on the 2 weeks preceding assessment of CMAI.

The CMAI (long-form version) will be assessed at Screening (Day −28 to Day −1), Baseline (Day 1), Visit 2 (Day 8), Visit 2.1 (Day 15), Visit 3 (Day 22), Visit 4 (Day 43), Visit 5 (Day 64), Visit 6 (Day 85), and Follow-up visit (for ET patients). The CMAI must be administered by the same rater at each visit.

### 4.2.2. Neuropsychiatric Inventory (NPI)

The NPI is a validated clinical instrument for evaluating psychopathology in a variety of disease settings, including dementia. The NPI is a retrospective caregiver-informant interview covering 12 neuropsychiatric symptom domains: delusions, hallucinations, agitation/aggression, depression/dysphoria, anxiety, elation/euphoria, apathy/indifference, disinhibition, irritability/lability, aberrant motor behavior, sleep and nighttime behavioral disorders, and appetite/eating disorders. Neuropsychiatric manifestations within a domain are collectively rated by the caregiver in terms of both frequency (1 to 4) and severity (1 to 3), yielding a composite symptom domain score (frequency×severity). Caregiver distress is rated for each positive neuropsychiatric symptom domain on a scale anchored by scores of 0 (not distressing at all) to 5 (extremely distressing).

The NPI will be administered to the patient's caregiver at Baseline (Day 1), Visit 3 (Day 22), Visit 4 (Day 43), Visit 5 (Day 64), and Visit 6 (Day 85). The Agitation/Aggression domain of the NPI will be administered to the patient's caregiver at Screening (Day −28 to Day −1), Visit 2 (Day 8), and Visit 2.1 (Day 15). The Agitation/Aggression domain in the NPI will be assessed as part of the total NPI as described above and the composite score obtained for this category will be recorded separately at Baseline (Day 1), Visit 3 (Day 22), Visit 4 (Day 43), Visit 5 (Day 64), and Visit 6 (Day 85). The recall period will be 2 weeks for all the visits. The NPI must be administered by the same rater at each visit. The NPI nursing-home version (NPI-NH) will be used for patients from in-patient or assisted living facilities.

### 4.2.3. Clinical Global Impression of Severity of Illness-Agitation (CGIS-Agitation)

The CGIS is an observer-rated scale that measures illness severity and is one of the most widely used brief assessment tools in psychiatry research.

The Early Clinical Drug Evaluation Unit (ECDEU) version of the CGIS is the most widely used format of this validated tool, and asks that the clinician rate the patient relative to their past experience with other patients with the same diagnosis, with or without collateral information. The CGIS has proved to be a robust measure of efficacy in many clinical drug trials and is easy and quick to administer, provided that the clinician knows the patient well.

Reliability and validity of CGI have been tested in multiple studies, including patients with dementia, schizophrenia and affective disorders. Overall, CGI showed high correlation (r: ˜90%) with other assessment instruments and it has also shown positive significant relationships and concurrent validity with other clinician's rating. In addition, the scale has good sensitivity to change over time.

The CGIS is a 7-point (1-7) scale (1=normal, not at all ill; 7=among the most extremely ill patients) and assesses severity of agitation in this study. The CGIS-Agitation will be assessed at Screening (Day −28 to Day −1), Baseline (Day 1), Visit 4 (Day 43), and Visit 6 (Day 85). The CGIS-Agitation must be administered by the same rater at each visit.

### 4.2.4. Patient Global Impression of Change (PGIC)

The PGIC is a 7-point (1-7) scale used to assess treatment response, and it is rated as: very much improved, much improved, minimally improved, no change, minimally worse, much worse, or very much worse.

The PGIC will be assessed and rated by the patient's caregiver at Visit 4 (Day 43) and Visit 6 (Day 85), and will focus on the patient's agitation.

### 4.2.5. EuroQol 5-Dimension 5-Level (EQ-5D-5L)

The EQ-5D-5L is a generic questionnaire measuring health-related quality of life and consists of a descriptive system and the EuroQol Visual Analogue Scale (EQ VAS). The descriptive system comprises 5 dimensions (mobility, self-care, usual activities, pain/discomfort, and anxiety/depression) and each dimension has 5 levels: no problems, slight problems, moderate problems, severe problems, and extreme problems. The EQ VAS records the respondent's self-rated health on a vertical, visual analogue scale where the endpoints are labeled ‘Best imaginable health state’ and ‘Worst imaginable health state’. This information can be used as a quantitative measure of health outcome as judged by the individual respondents. There are 2 versions of the EQ-5D-5L, a version rated by the patient and a version (EQ-5D-5L-proxy) rated by caregiver. The patient version will be rated only by patients with an MMSE score of ≥10 at the Baseline visit.

The EQ-5D-5L-proxy (and EQ-5D-5L for patients with MMSE≥10) will be assessed at Baseline (Day 1), Visit 4 (Day 43), and Visit 6 (Day 85).

### 4.3. Pharmacokinetics (PK)

Patients will have a blood sample collected between 1 to 4 hours after dosing at Baseline (Day 1), and at any time during Visit 4 (Day 43), Visit 5 (Day 64), and Visit 6 (Day 85) for the analysis of plasma levels of d6-DM, d6-DM metabolites and Q. Blood collection should usually occur after ECG and efficacy assessments.

Patients/caregivers should ensure that they record the time of last 2 doses prior to the clinic visit. Then the time when the patient was administered the last 2 doses of study medication prior to the clinic visit and the time of the blood draw will be recorded on the eCRF. Plasma samples will be separated by centrifugation and then frozen at −20° C. until assayed at the analytical unit.

### 4.4. Safety

### 4.4.1. Adverse Events

### 4.4.1.1. Definitions

An AE is any untoward medical occurrence or unintended change (physical, psychological, or behavioral) from the time ICF is signed, including inter-current illness, whether considered related to treatment or not. An AE can therefore be any unfavorable and unintended sign (including any clinically significant abnormal laboratory finding, for example), symptom, or disease temporally associated with the use of a medicinal product, whether or not considered related to the medicinal product. Changes associated with normal growth and development that do not vary in frequency or magnitude from that ordinarily anticipated clinically are not AEs (e.g., onset of menstruation occurring at a physiologically appropriate time).

Clinical AEs should be described by diagnosis and not by symptoms when possible (e.g., cold or seasonal allergies, instead of runny nose).

An overdose is a deliberate or inadvertent administration of a treatment at a dose higher than specified in the protocol and higher than known therapeutic doses. It must be reported irrespective of outcome even if toxic effects were not observed.

AEs will be graded on a 3-point scale and reported in detail as indicated on the eCRF:
Mild: easily tolerated, causing minimal discomfort and not interfering with normal everyday activities
Moderate: sufficiently discomforting to interfere with normal everyday activities
Severe: incapacitating and/or preventing normal everyday activities

The relationship of each AE to study medication should be determined by the investigator using the following explanations:
Not related: the event is clearly related to other factors such as the patient's clinical state, therapeutic interventions, or concomitant medications administered to the patient
Unlikely related: the event is most likely produced by other factors such as the patient's clinical state, therapeutic interventions, or concomitant medications administered to the patient; and does not follow a known response pattern to the study medication
Possibly related: the event follows a reasonable temporal sequence from the time of drug administration; and/or follows a known response pattern to the study medication; but could have been produced by other factors such as the patient's clinical state, therapeutic interventions, or concomitant medications administered to the patient
Related: the event follows a reasonable temporal sequence from the time of drug administration; and follows a known response pattern to the study medication; and cannot be reasonably explained by other factors such as the patient's clinical state, therapeutic interventions, or concomitant medications administered to the patient

### 4.4.1.2. Serious Adverse Events

A Serious Adverse Event (SAE) is any AE occurring at any dose that results in any of the following outcomes:
1. Death
2. Life-threatening experience (one that places the patient, in the view of the initial reporter, at immediate risk of death from the AE as it occurred, i.e., it does not include an AE that, had it occurred in a more severe form, might have caused death)
3. Persistent or significant disability/incapacity (disability is a substantial disruption of a person's ability to conduct normal life functions)
4. In-patient hospitalization or prolongation of hospitalization
5. Congenital anomaly/birth defect

Important medical events that may not result in death, or be life-threatening, or require hospitalization may be considered an SAE when, based upon appropriate medical judgment, they may jeopardize the patient or require medical or surgical intervention to prevent one of the outcomes listed in the definition.

The terms “cancer” and “overdose” are not necessarily considered SAEs, but if a patient experiences cancer or overdose, they are still reportable as AEs.

Pregnancy is not considered to be an AE or an SAE, but all pregnancies occurring during the study will be reported on the Pregnancy and Breastfeeding Exposure Form (PBEF). The site should follow-up each trimester with the patient/partner until the final outcome is known (i.e., normal delivery, abnormal delivery, spontaneous/voluntary/therapeutic abortion). Should a complication occur that meets the requirements for an AE or SAE, it must be reported within 24 hours of awareness. Patients who are pregnant or likely to become pregnant are excluded from this study. In the event a patient becomes pregnant during the study, study medication must be discontinued, a pregnancy report form must be completed to capture potential drug exposure during pregnancy, and the pregnancy must be reported within 24 hours of awareness.

A pregnancy report form must also be completed in the event that the partner of childbearing potential of a male patient in the study becomes pregnant within 30 days after his last dose of study medication or study completion, whichever is greater.

The term ‘severe’ is a measure of intensity; thus a severe AE is not necessarily serious. For example, nausea of several hours duration may be rated as severe, but may not be clinically serious.

### 4.4.2. Physical and Neurological Examinations

Physical and neurological examinations will be performed at Screening (Day −28 to Day −1) and Visit 6 (Day 85). The physical examination will include assessments of head, eyes, ears, nose, throat, lymph nodes, skin, extremities, respiratory, gastrointestinal, musculoskeletal, cardiovascular, and nervous systems. The neurological examination will include assessments of mental status, cranial nerves, motor system, reflexes, coordination, gait and station, and sensory system. The physical and neurological examinations should be performed by the same person each time, whenever possible.

Physical and neurological examination abnormalities determined by the investigator to be clinically significant at Screening should be recorded as medical history.

Any clinically significant changes in physical and neurological examination findings from the screening examination should be recorded as AEs.

### 4.4.3. Electrocardiograms

A resting 12-lead ECG will be performed at all clinic visits except on Day 8 (Visit 2). At Screening (Day −28 to Day −1), ECGs will be performed in triplicate. At Baseline (Day 1), two ECGs will be performed; one prior to study medication dosing and one at least 1 hour after dosing. ECG equipment will be provided by the central reader. ECG data will be recorded at the study center and will include general findings, heart rate (beats/minute) QRS complex and PR and QTc intervals (milliseconds). Results will be provided by the central reader to the investigators within 24 hours. ECG abnormalities present at Screening will be recorded as medical history. Any changes from the ECG status at Screening Visit that are deemed to be clinically significant by the investigator should be captured as AEs. Any clinically significant abnormal ECG should be discussed with the study MM and, if necessary be repeated within a 1-week period.

For eligibility to enroll in the study, the QTcF assessment of ECGs conducted at Screening will be based on the central review. The assessment of ECGs conducted at Baseline will be based on the machine read and investigator evaluation of the read. If the Baseline pre-dose ECG QTcF result from the machine read is exclusionary, the patient should not be dosed and the MM should be consulted.

### 4.4.4. Sheehan Suicidality Tracking Scale (S-STS)

The S-STS is a prospective scale that assesses treatment-emergent suicidal thoughts and behaviors. Each item of the S-STS is scored on a 5-point Likert scale (0=not at all; 1=a little; 2=moderate; 3=very; and 4=extremely). The Sheehan-STS can be analyzed as individual item scores, suicidal ideation subscale score, suicidal behavior subscale score, or total score. For the screening visit, the timeframe for the items on the scale will be ‘in the past 6 months’ and for all other visits it will be ‘since last visit’.

The S-STS will be assessed at Screening (Day −28 to Day −1), Baseline (Day 1), Visit 2 (Day 8), Visit 2.1 (Day 15), Visit 3 (Day 22), Visit 4 (Day 43), Visit 5 (Day 64), Visit 6 (Day 85), and Follow-up visit (for ET patients). Any change in the S-STS score indicating the presence of suicidality should be evaluated by the investigator and reported to the MM.

### 4.4.5. Mini Mental State Examination (MMSE)

The MMSE is a brief 30-point questionnaire test that is used to screen for cognitive impairment. It is commonly used in medicine to screen for dementia. It is also used to estimate the severity of cognitive impairment at a specific time and to follow the course of cognitive changes in an individual over time, thus making it an effective way to document an individual's response to treatment. The MMSE scale comprises 11 questions or simple tasks concerning orientation, memory, attention, and language to evaluate the patient's cognitive state. It requires only 5 to 10 minutes for a trained rater to administer it.

The MMSE will be assessed at Screening (Day −28 to Day −1), Baseline (Day 1), and Visit 6 (Day 85).

### 4.4.6. Epworth Sleepiness Scale (ESS)

The ESS is an 8-item questionnaire that is used to measure sleepiness by rating the probability of falling asleep on 8 different situations that most people engage in during the day. The questions are rated on a 4 point scale (0 to 3) where 0=would never doze, 1=slight chance of dozing, 2=moderate chance of dozing, and 3=high chance of dozing. A total score of 0 to 9 is considered to be normal.

The ESS will be assessed at Baseline (Day 1), Visit 4 (Day 43), and Visit 6 (Day 85) for patients with an MMSE score of ≥10 at the Baseline visit.

### 4.4.6.1. Screening Visit (Days −28 to −1, +3-Day Window)

The following procedures will be performed at Screening (within 28 days prior to Day 1). The screening period may be extended after discussion with and approval by the MM. In the event that a patient is rescreened for enrollment, new informed consent and/or assent documents must be signed, new patient number assigned and all screening procedures repeated.
1. The investigator will provide the patients, authorized representatives and/or their caregivers with informed consent and/or assent documents and will explain the rationale for the study, providing ample time for participants, authorized representatives, and/or caregivers to ask questions.
2. Medical history, including patient demographics, any prior and concomitant medications (including OTC, vitamins, and supplements), nondrug therapies, and any nonpharmacological interventions for the treatment of agitation will be reviewed and recorded.
3. Review inclusion/exclusion criteria (protocol eligibility form).
4. Vital signs will be measured and recorded.
5. Physical and neurological examination will be performed.
6. Risk assessment for falls will be performed (worksheet and TUG test).
7. A resting 12-lead ECG will be performed in triplicate.
8. A blood and urine specimen will be collected for safety laboratory assessments.
9. A urine pregnancy test will be performed for females of childbearing potential only.
10. The following assessments will be completed.
   MMSE; a score between 6 and 26 (inclusive) is required for study entry
   CMAI
   NPI-Agitation/Aggression domain
   CGIS-Agitation; a score of ≥4 is required for study entry
   S-STS
   CSDD; a score of <10 is required for study entry
11. Register the screening visit in IWRS

Following screening procedures for assessment of inclusion and exclusion criteria, the site will complete a Protocol Eligibility Form (PEF) and submit to the MM for review and approval. Patients deemed eligible by the PI and the MM will be randomized into the study should they continue to qualify at the Baseline (Day 1) visit. Patients who have ECG or laboratory test results outside of the reference normal range that the investigator considers to be clinically significant, and may put the patient at a higher risk for study participation, will not be enrolled.

### 4.4.6.2. Baseline Visit (Day 1)

The Baseline visit (Day 1) should occur in the morning. The following procedures will be performed.

### Before Dosing:

1. Inclusion/exclusion criteria will be reviewed.
2. Caregivers will be queried regarding AEs, concomitant medication use (including OTC, vitamins, and supplements), nondrug therapies, and concomitant nonpharmacological interventions for the treatment of agitation.
3. Vital signs, height, and weight will be measured and recorded (height and weight can be measured during the visit on the same day after dosing).
4. A resting pre-dose 12-lead ECG will be performed.
5. A urine pregnancy test will be performed for females of childbearing potential only.
6. The following assessments will be completed:
   MMSE
   CMAI
   NPI
   CGIS-Agitation
   EQ-5D-5L-proxy (and EQ-5D-5L for patients with an MMSE score of ≥10 at baseline)
   S-STS
   ESS (only for patients with an MMSE score of ≥10 at baseline)

Patients will be randomized once it is determined that they satisfy all of the inclusion and none of the exclusion criteria (on the basis of the screening and baseline assessments described above) and will be assigned with a study medication kit number via IWRS.

### Study Medication Dosing:

The first dose of study medication will be administered from the AM strip of blister card at the clinic regardless of the time of day.

### After Dosing:

1. A resting post-dose 12-lead ECG will be performed at least 1 hour after taking the morning dose of study medication.
2. A blood specimen will be collected within 1 to 4 hours after the first dose of study medication for PK analysis and for CYP2D6 genotyping.
3. The caregiver will be queried regarding AEs.
4. Patient Diary Card and sufficient study medication for a 3-week treatment period will be dispensed.

### 4.4.6.3. Visit 2 (Day 8+3-Day Window)

Visit 2 (Day 8) dose of study medication can be administered at home; the time of dosing should be noted by the patient/caregiver.

The following procedures will be performed.
1. The caregiver will be queried regarding AEs, concomitant medication use (including OTC, vitamins, and supplements), nondrug therapies, and concomitant nonpharmacological interventions for the treatment of agitation.
2. Vital signs will be measured and recorded.
3. The following assessments will be completed:
   CMAI
   NPI-Agitation/Aggression domain
   S-STS
4. Register study visit in IWRS
5. Unused study medication will be accounted for compliance and the blister card returned to the patient.
6. Patient's Diary Card will be reviewed for compliance and returned to the patient.

### 4.4.6.4. Visit 2.1 (Day 15±3-Day Window)

Visit 2.1 (Day 15) dose of study medication can be administered at home; the time of dosing should be noted by the patient/caregiver.

The following procedures will be performed.
1. A resting 12-lead ECG will be performed.
2. The caregiver will be queried regarding AEs, concomitant medication use (including OTC, vitamins, and supplements), nondrug therapies, and concomitant nonpharmacological interventions for the treatment of agitation.
3. Vital signs will be measured and recorded.
4. The following assessments will be completed:
   CMAI
   NPI-Agitation/Aggression domain
   S-STS
5. Register study visit in IWRS
6. Unused study medication will be accounted for compliance and the blister card returned to the patient.
7. Patient's Diary Card will be reviewed for compliance and returned to the patient.

### 4.4.6.5. Visit 3 (Day 22±3-Day Window)

Visit 3 (Day 22) dose of study medication can be administered at home; the time of dosing for the 2 doses prior to the study visit should be noted by the patient/caregiver.

The following procedures will be performed:
1. The caregiver will be queried regarding AEs, concomitant medication use (including OTC, vitamins, and supplements), nondrug therapies, and concomitant nonpharmacological interventions for the treatment of agitation.
2. Vital signs will be measured and recorded
3. Returned, unused study medication will be accounted for compliance.
4. Patient's Diary Card will be collected and reviewed for compliance.
5. The following assessments will be completed:
   CMAI
   NPI
   S-STS
6. Register study visit in IWRS
7. A resting 12-lead ECG will be performed.
8. A blood and urine specimen will be collected for safety laboratory assessments.
9. Diary Card and sufficient study medication for a 3-week treatment period will be dispensed.

### 4.4.6.6. Visit 4 (Day 43±3-Day Window)

Visit 4 (Day 43) should occur in the morning. The morning dose of study medication can be administered at home; the time of dosing for the 2 doses prior to the study visit should be noted by the patient/caregiver.

The following procedures will be performed.
1. Caregivers will be queried regarding AEs, concomitant medication use (including OTC, vitamins, and supplements), nondrug therapies, and concomitant nonpharmacological interventions for the treatment of agitation.
2. Vital signs will be measured and recorded.
3. Patient's Diary Card will be collected and reviewed for compliance.
4. A urine sample will be collected for urinalysis.
5. A urine pregnancy test will be performed for females of childbearing potential only.
6. Returned, unused study medication will be accounted for compliance.
7. The following assessments will be completed:
   CMAI
   NPI
   CGIS-Agitation
8. Register study visit in IWRS
9. A resting 12-lead ECG will be performed
10. A blood specimen will be collected for PK analysis and for safety laboratory assessments. The time of sample collection to be noted.
11. The following assessments will be completed:
   EQ-5D-5L-proxy (and EQ-5D-5L for patients with an MMSE score of ≥10 at baseline)
   S-STS
   PGIC
   ESS (only for patients with an MMSE score of ≥10 at baseline)
12. Patient Diary Card and sufficient study medication for a 3-week treatment period will be dispensed.

### 4.4.6.7. Visit 5 (Day 64±3-Day Window)

Visit 5 (Day 64) should occur in the morning. The morning dose of study medication can be administered at home; the time of dosing for the 2 doses prior to the study visit should be noted by the patient/caregiver.

The following procedures will be performed.
1. The caregiver will be queried regarding AEs, concomitant medication use (including OTC, vitamins, and supplements), nondrug therapies, and concomitant nonpharmacological interventions for the treatment of agitation.
2. Vital signs will be measured and recorded.
3. Returned, unused study medication will be accounted for compliance.
4. Patient's Diary Card will be collected and reviewed for compliance.
5. A resting 12-lead ECG will be performed
6. A blood specimen will be collected for PK analysis and for safety laboratory assessments.
7. A urine sample will be collected for urinalysis.
8. The following assessments will be completed.
   CMAI
   NPI
   S-STS
9. Register study visit in IWRS
10. Patient Diary Card and sufficient study medication for a 3-week treatment period will be dispensed.

### 4.4.6.8. Visit 6 (Day 85±3-Day Window)/Early Termination

Visit 6 (Day 85) should occur in the morning. The morning dose of study medication can be administered at home; the time of dosing for the 2 doses prior to the study visit should be noted by the patient/caregiver.

Patients who withdraw prior to study completion are required to complete study procedures as listed in Visit 6 within 48 hours of the last dose of study medication. PK samples do not need to be collected for patients who terminate early.
1. A urine sample will be collected for urinalysis.
2. Urinary pregnancy test will be performed in patients of childbearing potential.
3. The caregiver will be queried regarding AEs, concomitant medication use (including OTC, vitamins, and supplements), nondrug therapies, and concomitant nonpharmacological interventions for the treatment of agitation.
4. Returned, unused study medication will be accounted for compliance.
5. Patient's Diary Card will be collected and reviewed.
6. Vital signs and weight will be measured and recorded.
7. Physical and neurological examination will be performed.
8. The following assessments will be completed:
   MMSE
   CMAI
   NPI
   CGIS-Agitation
   EQ-5D-5L-proxy (and EQ-5D-5L for patients with an MMSE score of 10 at baseline)
   S-STS
   PGIC
   ESS (only for patients with an MMSE score of ≥10 at baseline)
9. A resting 12-lead ECG will be performed.
10. A blood specimen will be collected for PK analysis and for safety laboratory assessments. Time of sample collection to be noted.
11. Register study visit in IWRS
12. Any previously reported and not yet resolved AE and any newly reported AE at the time of this visit, will be followed-up for up to 30 days after the last dose of study medication.

### 5. Statistical Methods

### 5.1. Analysis Populations

Three analysis populations will be used; modified intent-to-treat (mITT), intent-to-treat (ITT), and safety.
1. mITT: The mITT population includes all patients randomized in the study who had at least one post-baseline efficacy assessment. The mITT population will be used for all analyses of efficacy. Patients will be included in the treatment group to which they were randomized regardless of treatment received.
2. ITT: The ITT population includes all randomized patients in the study. The ITT population will be used for exploratory efficacy analyses.
3. Safety: The safety population includes all patients who received study treatment. The safety population will be used for all analyses of safety. Patients will be included in the treatment group based on the actual treatment received.

### 5.2. Efficacy Analysis

### 5.2.1. Study Endpoints

Primary efficacy endpoint:

The primary efficacy endpoint is the change from Baseline to Week 12 (Day 85) in the CMAI total score.

Secondary efficacy endpoint:

The secondary efficacy endpoint is the change from Baseline to Week 12 (Day 85) in the CGIS-Agitation.

The other efficacy endpoints are the change from Baseline to Week 12 (Day 85) in the following measures:
NPI-Agitation/Aggression domain score and Caregiver Distress score
NPI—Aberrant Motor Behavior domain
NPI—Irritability/Lability domain
Total NPI
PGIC
EQ-5D-5L

### 5.2.2. Primary Efficacy Analysis

The primary efficacy endpoint is the change from Baseline to Day 85 (Week 12) in the CMAI total score.

For the primary efficacy analysis, the null hypothesis is that there is no treatment effect between AVP-786-42.63/4.9 and placebo during the study and it will be tested against the alternative that there is a treatment effect. Similar hypotheses apply to the comparison of the AVP-786-28/4.9 vs. placebo. The treatment effect will be analyzed by using a linear mixed effects model repeated measures (MMRM) with fixed effects for treatment, visit, treatment-by-visit interaction, baseline-by-visit interaction, and baseline covariates which include baseline value and other factors as appropriate. An unstructured covariance model will be used.

In addition, the primary endpoint will also be analyzed with missing data imputed by other statistical methods, such as multiple imputation.

### 5.2.3. Secondary and Other Efficacy Analyses

The secondary and other efficacy endpoints include change from Baseline to Week 12 (Day 85) for the following efficacy measures: CGIS-Agitation, NPI-Agitation/Aggression domain score and Caregiver Distress score, NPI—Aberrant Motor Behavior domain score, NPI—Irritability/Lability domain score, PGIC, EQ-5D-5L, and total NPI.

Treatment comparison tests using similar MMRM method as the primary efficacy analysis will be performed when appropriate.

### 5.3. Pharmacokinetic Analyses

Plasma concentrations of d6-DM, Q and metabolites will be measured, and results will be summarized descriptively overall and by cytochrome P450 isoenzyme 2D6 (CYP2D6) metabolizer group.

### 5.4. CYP2D6 Genotype

Genotype information will be used to classify patients as poor metabolizers, intermediate metabolizers, extensive metabolizers, or ultra-rapid metabolizers of d6-DM.

### 5.5. Safety Analysis

Safety will be assessed by the following measurements: AEs, physical and neurological examination, vital signs, urine pregnancy test, clinical laboratory assessments, resting 12-lead ECG, S-STS, MMSE, and ESS.

Safety analyses will consist of data summaries for biological parameters and AEs. Safety analyses will be tabulated by treatment.

### Appendix 2. Prohibited Concomitant Medications

Patients who are currently taking, or have taken any of the following types of drugs, within 2 weeks or 5 half-lives, whichever is longer, prior to the initiation of the study medication administration, are to be excluded.
A. Certain drugs that may increase Q levels (exclusion does not include topical medications unless applied under occlusive dressing or other technique that is intended to increase systemic absorption):
   amiodarone
   antifungals (e.g., fluconazole, itraconazole, ketoconazole, posaconazole, voriconazole)
   carbonic anhydrase inhibitors (strong inhibitors are prohibited: topiramate is allowed)
   cimetidine
   delavirdine
   diltiazem
   itraconazole
   ketoconazole
   macrolide antibiotics (e.g., erythromycin, azithromycin, clarithromycin, telithromycin, dirithromycin, roxithromycin)
   mexiletine
   nefazodone
   protease inhibitors (e.g., amprenavir, atazanavir, fosamprenavir, indinavir, ritonavir, saquinavir)
   ranolazine
   verapamil
B. Certain drugs that may have increased plasma levels if co-administered with Q:
   atomoxetine
   dextromethorphan (over-the-counter [OTC] and prescription)
   tricyclic antidepressants (TCA; e.g., amitriptyline, amoxapine, clomipramine, desipramine, doxepin, imipramine, nortriptyline, protriptyline)
C. Drugs that are related to Q:
   mefloquine
   quinidine
   quinine
D. Monoamine oxidase inhibitors (MAOIs) (may increase the risk of serotonin syndrome) Patients should allow at least 14 days after stopping study medication before starting an MAOI.
E. CYP3A4 inducers that may decrease DM or Q plasma levels:
   apalutamide
   carbamazepine
   dexamethasone
   enzalutamide
   fosphenytoin
   lumacaftor
   mitotane
   pentobarbital
   phenobarbital
   phenytoin
   primidone
   rifampicin
   rifamycin
   rifaximin
   St. John's wort
F. Certain drugs that may be prescribed for the treatment of agitation or other indications:
   Benzodiazepines (e.g., lorazepam)
   phenothiazines (e.g., chlorpromazine, fluphenazine, levomepromazine, methotrimeprazine, mesoridazine, pencyanzine, perphenazine, prochlorperazine, promazine, thioridazine, thiothixene, trifluoperazine, triflupromazine)
   typical antipsychotics (e.g., droperidol, haloperidol, loxapine, molindone, pimozide, zuclopenthixol)
   clozapine

### Medications Containing Dextromethorphan (Over-the-Counter [OTC] and Prescription)

### Example 3

A Phase 3, multicenter, randomized, double-blind, placebo-controlled study to assess the efficacy, safety, and tolerability of AVP-786 (deuterated [d6]-dextromethorphan hydrobromide [d6-dm]/quinidine sulfate [Q]) for the treatment of agitation in patients with dementia of the Alzheimer's type

### 1. List of Abbreviations and Definitions of Terms

The following abbreviations and specialized terms are used in this Example 3.

TABLE 6
Abbreviations and Specialized Terms

| Abbreviation or | |
|---|---|
| Specialized Term | Explanation |
| ADAS-cog | Alzheimer's Disease Assessment |
| | Scale-cognitive subscale |
| ADCS-CGIC | Alzheimer’s Disease Cooperative |
| | Study-Clinical Global Impression of |
| | Change |
| ADCS-CGIC-Overall | Alzheimer’s Disease Cooperative |
| | Study-Clinical Global Impression of |
| | Change for Overall Clinical Status |
| AE | adverse event |
| ALT | alanine aminotransferase |
| ANCOVA | analysis of covariance |
| AR1 | autoregressive of order 1 |
| AST | aspartate aminotransferase |
| AUC | area under the concentration-time curve |
| Avanir | Avanir Pharmaceuticals, Inc (the Sponsor) |
| AVP-786 | deudextromethorphan hydrobromide |
| | [d6-DM]/quinidine sulfate [Q] |
| AVP-786-18 | AVP-786 containing d6-DM 18 mg/ |
| | Q 4.9 mg |
| AVP-786-28 | AVP-786 containing d6-DM 28 mg/ |
| | Q 4.9 mg |
| BUN | blood urea nitrogen |
| CFR | US Code of Federal Regulations |
| CGIC | Clinical Global Impression of Change |
| CGIS | Clinical Global Impression of Severity |
| CGIS-Agitation | Clinical Global Impression of Severity of |
| | Illness scale for Agitation |
| CK | creatine kinase |
| CMAI | Cohen-Mansfield Agitation Inventory |
| Cₘₐₓ | maximum plasma concentration |
| CNS | central nervous system |
| CS | compound symmetry |
| CSDD | Cornell Scale for Depression in Dementia |
| CYP | cytochrome P450 |
| d3-3-MM | d3-3-methoxymorphinan |
| d3-DX | deuterated dextrorphan |
| d6-DM | deudextromethorphan (hydrobromide) |
| DEMQOL | Dementia Quality of Life |
| DM | dextromethorphan hydrobromide |
| DSMB | Data Safety Monitoring Board |
| ECG | electrocardiogram |
| ECDEU | Early Clinical Drug Evaluation Unit |
| eCRF | electronic case report form |
| EP | European Pharmacopoeia |
| EQ-5D-5L | EuroQol 5-Dimension 5-Level |
| ESS | Epworth Sleepiness Scale |
| FDA | Food and Drue Administration |
| FWE | family-wise error |
| GCP | Good Clinical Practice |
| GGT | gamma-glutamyl transferase |
| GMHR | General Medical Health Rating |
| HbA1c | glycosylated hemoglobin |
| ICF | informed consent form |
| ICH | International Council for Harmonisation |
| IP | investigational product |
| IRB | Institutional Review Board |
| ITT | intent-to-treat |
| IWRS | interactive web-response system |
| LAR | Legal Authorized Representative |
| LDH | lactate dehydrogenase |
| LOCF | last observation carried forward |
| mADCS-CGIC | Modified Alzheimer’s Disease |
| | Cooperative Study-Clinical |
| | Global Impression of Change |
| mADCS-CGIC- | Modified Alzheimer’s Disease |
| Agitation | Cooperative Study-Clinical |
| | Global Impression of Change scale for |
| | Agitation |
| MAOI | monoamine oxidase inhibitors |
| MedDRA | Medical Dictionary for Regulatory |
| | Activities |
| mITT | modified intent-to-treat |
| MMRM | mixed model repeated measures |
| MMSE | Mini Mental State Examination |
| MNAR | missing not at random |
| NIA-AA | National Institute on Aging-Alzheimer’s |
| | Association |
| NPI | Neuropsychiatric Inventory |
| NPI-NH | Neuropsychiatric Inventory nursing home |
| | version |
| OLS | ordinary least squares |
| PD | pharmacodynamic |
| PGIC | Patient Global Impression of Change |
| PK | pharmacokinetic |
| PMM | Pattern Mixture Models |
| PT | preferred term |
| PVC | premature ventricular contractions |
| Q | quinidine sulfate |
| QOL | quality of life |
| QTcF | QTc by Fridericia's formula |
| RBC | red blood cell |
| RUD | Resource Utilization in Dementia |
| SAE | serious adverse event |
| SAP | statistical analysis plan |
| SD | standard deviation |
| SNRI | serotonin-norepinephrine reuptake |
| | inhibitor |
| SOC | system organ class |
| SPCD | sequential parallel comparison design |
| SSRI | selective serotonin reuptake inhibitors |
| S-STS | Sheehan Suicidality Tracking Scale |
| SUR | seemingly unrelated regression |
| T3 | triiodothyronine |
| T4 | thyroxine |
| TEAE | treatment-emergent adverse event |
| TSH | thyroid-stimulating hormone |
| TUG | Timed Up and Go |
| USP | United States Pharmacopoeia |
| WBC | white blood cell |
| WOCF | worst observation carried forward |
| ZBI | Zarit Burden Interview |

### 2. Introduction

### 2.1. AVP-786

AVP-786 is a combination product of deudextromethorphan hydrobromide (d6-DM), a central nervous system (CNS) active agent, and quinidine sulfate (Q), used as an inhibitor of d6-DM metabolism via the cytochrome P450 (CYP) liver isoenzyme 2D6 (CYP2D6). The demonstrated receptor pharmacology of d6-DM may underlie the potential clinical benefit for agitation in patients with dementia of the Alzheimer's type. d6-DM binds to receptors responsible for modulation of glutamate and monoamines, and also binds to the sigma-1 receptor; these interactions may be key to CNS therapeutics.

### 3. Investigational Plan

### 3.1. Overall Study Design and Plan: Description

This was a Phase 3, multicenter, randomized, double-blind, placebo-controlled, sequential parallel comparison design (SPCD) study with a 12-week treatment duration. The treatment period was divided equally into two 6-week stages (Stage 1 and Stage 2), with Screening from Day −28 to −1, Stage 1 from Day 1 to 42, and Stage 2 from Day 43 to 85.

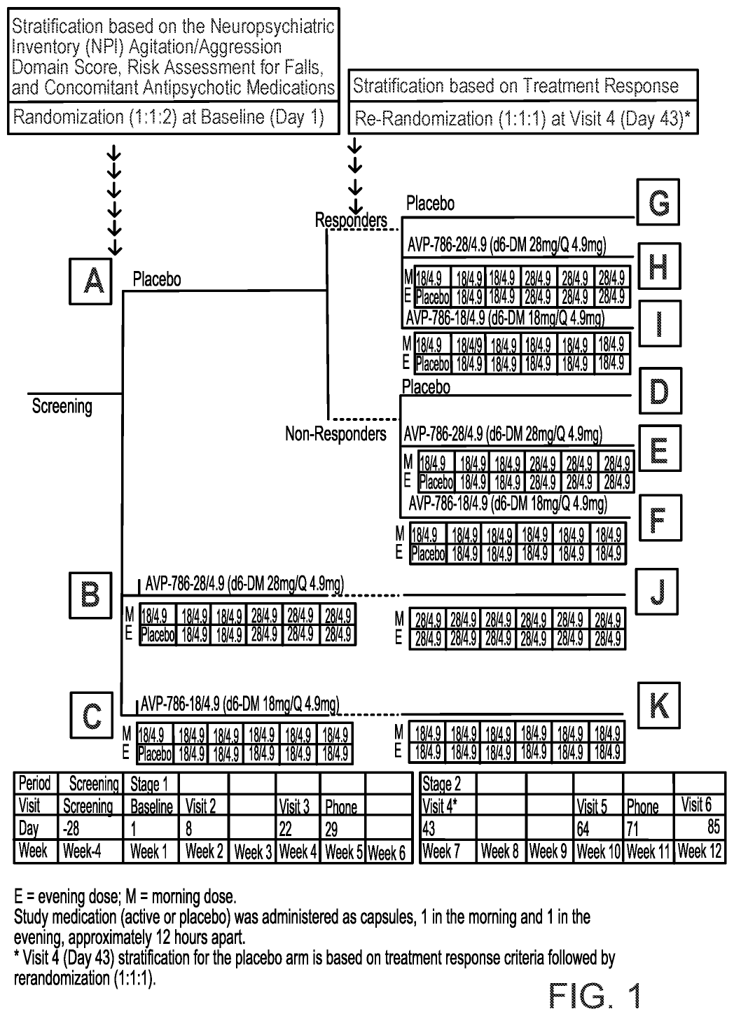

The SPCD is illustrated in FIG. 1. For Stage 1, patients were to be randomized 1:1:2 to AVP-786 containing d6-DM 18 mg/Q 4.9 mg (AVP-786-18), AVP-786 containing d6-DM 28 mg/Q 4.9 mg (AVP-786-28), or placebo for 6 weeks, with randomization stratified by the Neuropsychiatric Inventory (NPI) Agitation/Aggression Domain score (≤6 vs >6), risk assessment for falls (normal/mild vs moderate/severe), and concomitant use of antipsychotic medications (yes vs no). For Stage 2, patients randomized to active treatment in Stage 1 (AVP-786-18 or AVP-786-28) were to continue receiving the same treatment; those randomized to placebo in Stage 1 were to be rerandomized, 1:1:1 to AVP-786-18, AVP-786-28, or placebo. Randomization in Stage 2 was stratified by placebo response in Stage 1 (yes vs no), and all patients were to be treated for an additional 6 weeks. In both stages, study medication (active or placebo) was to be administered orally twice daily (I capsule in the morning and 1 capsule in the evening approximately 12 hours apart) throughout the treatment period. Patients beginning active treatment in both stages were titrated to their randomized dose.

There were 8 scheduled clinic visits planned, including a Screening Visit. Patients were to attend clinic visits at Screening, Baseline (Day 1), and on Days 8 (Visit 2/Week 1), 15 (Visit 2.1/Week 2), 22 (Visit 3/Week 3), 43 (Visit 4/Week 6), 64 (Visit 5/Week 9), and 85 (Visit 6/Week 12). Two safety phone calls were scheduled on Days 29 (Week 4) and 71 (Week 10). For patients who did not roll over into the extension study, a safety phone call was scheduled 30 days after last dose of study medication.

The SPCD was to be kept blinded from patients and study personnel at the investigative centers, and the rerandomization and criteria for Stage 2 were also blinded. Centers were provided with a masked protocol describing the study as a parallel-group study with a 12-week treatment duration (with the exception of rerandomization, all procedures were to be performed as described in the blinded protocol).

The SPCD, which was developed to address the large magnitude of placebo response often noted in psychopharmacological studies, allows for efficacy analyses for each stage as well for the combined stages as a weighted SPCD analysis. For Stage 1, the primary analysis was to include all patients in the modified intent-to-treat (mITT) population, defined as all randomized patients with at least 1 postbaseline efficacy assessment, and each active treatment group (Groups B and C in FIG. 1) was compared to placebo (Group A) using a hierarchical testing approach to preserve an alpha of 0.05. For Stage 2, the primary analysis was to include only those mITT patients who had been randomized to placebo in Stage 1 and had not demonstrated a placebo response (Placebo Nonresponders), and each active treatment group (Groups E and F in FIG. 1) was to be compared to placebo in this subgroup (Group D) using a hierarchical testing approach to preserve an alpha of 0.05. For the hierarchical testing, the higher dose (Group B in Stage 1 and Group E in Stage 2) was tested first. The null hypothesis was that there was no difference in the change in the primary efficacy endpoint (Cohen-Mansfield Agitation Inventory [CMAI] Total score) between AVP-786 and placebo in Stage 1 and Stage 2 for each dose, and it was tested against the alternative hypothesis that there was a treatment effect in at least 1 of the 2 stages.

A third comparison was performed comparing the results for patients who received active treatment for both stages of the study (12 weeks; Groups B/J and C/K in FIG. 1). versus those who received placebo for both stages (i.e., patients who were randomized to AVP-786-18 or AVP-786-28 during Stages 1 and 2 versus patients who were randomized to placebo during Stages 1 and 2; Groups A, D, and G).

### 3.2. Discussion of Study Design, Including the Choice of Control Groups

The randomized, placebo-controlled, double-blind, SPCD was developed to reduce sources of bias. Potentially high responses observed among placebo-treated patients can constitute a significant challenge for drug development in studies of behavioral and psychiatric disorders. The SPCD is essentially comprised of 2 randomized trials (stages) run one after another; Stage 1 includes all patients randomized and Stage 2 rerandomizes those who were Nonresponders to placebo during Stage 1 to active drug or placebo. The expectation is that signal detection will be enhanced by including data from Placebo Nonresponders in the primary analysis, which is comprised of pooled data from Stage 1 and Stage 2. The SPCD was created to increase the power of a study to identify a clinically significant effect in situations where there may be a large placebo effect, particularly in psychopharmacological studies. The design, its utility, and statistical considerations have been described previously.

### 3.3. Selection of Study Population

### 3.3.1. Inclusion Criteria

For inclusion into the trial, patients were required to fulfill all of the following criteria.
1. Males and females 50 to 90 years of age, inclusive, at the time of informed consent.
2. Diagnosis of probable Alzheimer's disease according to the 2011 National Institute on Aging-Alzheimer's Association (NIA-AA) working groups criteria. Either outpatients or residents of an assisted-living facility or a skilled nursing home.
3. The patient had clinically significant, moderate/severe agitation, at the time of Screening and for at least 2 weeks prior to randomization, that interfered with daily routine and for which a prescription medication was indicated, in the opinion of the Investigator.
4. The diagnosis of agitation had to meet the International Psychogeriatric Association provisional definition of agitation.
5. Clinical Global Impression Severity (CGIS)-Agitation score ≥4 (moderately ill) at Screening and Baseline.
6. Mini Mental State Examination (MMSE) score between 6 and 26 (inclusive) at Screening and Baseline.
7. The patient had stable cardiac, pulmonary, hepatic, and renal function.
8. The patient had an ECG (obtained within the past month prior to randomization and evaluated by a central ECG reader) with no clinically significant findings.
9. If female of childbearing potential, had to have been practicing a medically acceptable method of birth control for at least 1 month prior to randomization and continue with the same method during the entire study duration (oral contraceptive tablets, hormonal implant device, hormone patch, intrauterine device, diaphragm and contraceptive cream or foam, condom with spermicide, or abstinence) or to have been surgically sterile or postmenopausal.
10. Use of medication for the treatment of Alzheimer's disease (e.g., donepezil, rivastigmine, galantamine, memantine) was allowed provided the dose had been stable for at least 3 months prior to randomization.
11. Concomitant use of antidepressants such as selective serotonin reuptake inhibitors (SSRIs; e.g., fluoxetine, sertraline, citalopram), serotonin-norepinephrine reuptake inhibitors (SNRIs; e.g., venlafaxine, desvenlafaxine, duloxetine) was allowed, provided the dose had been stable for at least 1 month prior to randomization and was within the Package Insert guidance for that medication. Paroxetine, a CYP2D6 substrate, was allowed provided the dose did not exceed 10 mg/day.
12. Concomitant use of hypnotics at bedtime (e.g., eszopiclone, zolpidem, zaleplon, trazodone [up to 50 mg/day]) for the nighttime treatment of insomnia was allowed, provided the dose had been stable for at least 1 month prior to randomization and remained stable throughout the study. In addition, concomitant use of short acting benzodiazepines (e.g., midazolam, oxazepam, low dose alprazolam [up to 0.5 mg/day]) for behavioral disturbances was allowed.
13. Patients concurrently taking allowed medications for the treatment of agitation secondary to Alzheimer's disease (e.g., atypical antipsychotics, antidepressants, buspirone) were eligible provided they had been on a stable dose for at least 2 weeks prior to Screening and at least 1 month prior to randomization.
14. Patient had to not show current and significant symptoms of a depressive disorder and had to have a score <10 in the Cornell Scale for Depression in Dementia (CSDD) at Screening.
15. Patient had to have no history or current clinical symptoms of schizophrenia, schizoaffective disorder, or bipolar disorder, as defined in the Diagnostic and Statistical Manual of Mental Disorders, 4th Edition, Text Revision.
16. Caregiver had to be willing and able to comply with study procedures, including not administering any prohibited medications during the course of the study.
17. Patient/caregiver had to be willing to sign and receive a copy of patient/caregiver ICF after the nature and risks of study participation had been fully explained. Patients who were not capable of signing the ICF but were able to provide assent, or the patient's authorized representative agreed to participation (for patients unable to provide assent) were allowed.

### 3.3.2. Exclusion Criteria

Any of the following was regarded as a criterion for exclusion from the trial.
1. Caregiver was unwilling or unable, in the opinion of the Investigator, to comply with study instructions.
2. Patient had dementia predominantly of non-Alzheimer's type (e.g., vascular dementia, frontotemporal dementia, Parkinson's disease, substance-induced dementia).
3. Patients with symptoms of agitation that were not secondary to Alzheimer's disease (e.g., secondary to pain, other psychiatric disorder, or delirium).
4. Patients with myasthenia gravis (contraindication for Q).
5. Patients with any personal history of complete heart block, QTc prolongation, or torsades de pointes.
   Screening and Baseline QTc by Fridericia's formula (QTcF) of >450 msec for males and >470 msec for females based on central review unless due to ventricular pacing Presence of premature ventricular contractions (PVCs) as evaluated by a central reader and deemed clinically significant by the Investigator
6. Patients with any family history of congenital QT interval prolongation syndrome.
7. Patients with known hypersensitivity to DM, Q, opiate drugs (codeine, etc.), or any other ingredient of the study medication.
8. Patients with history of allergy to benzodiazepines (e.g., lorazepam).
9. Patients who had ever received DM co-administered with Q.
10. Patients who had been taking disallowed concomitant medications within 2 weeks or 5 half-lives, whichever was longer, prior to Baseline.
11. Patients with co-existent clinically significant or unstable systemic diseases that could confound the interpretation of the safety results of the study (e.g., malignancy [except skin basal-cell carcinoma or untreated prostate cancer], poorly controlled diabetes, poorly controlled hypertension, unstable pulmonary, renal or hepatic disease, unstable ischemic cardiac disease, dilated cardiomyopathy, or unstable valvular heart disease). Certain other non-metastatic cancer could have been allowed. Each case was to be evaluated individually with the Medical Monitor.
12. Patients who were currently participating in, or who had participated in other interventional (drug or device) clinical study within 30 days of Baseline.
13. Patients with history of postural syncope or any history of unexplained syncope (evaluated on a case by case basis) within 12 months of Baseline.
14. Patients with a history of substance and/or alcohol abuse within the past 1 year.
15. Patients determined to have a high imminent risk of falls during the study based on a clinical evaluation by the Investigator.
16. Patients with evidence of serious risk of suicide at Screening and Baseline based on the Sheehan Suicidality Tracking Scale (S-STS), i.e., a score of 3 or 4 on any 1 question 2 through 6 or 11 or a score of 2 or higher on any 1 questions 1a, 7 through 10, or 12, or who, in the opinion of the Investigator, present a serious risk of suicide.

### 3.3.3. Removal of Patients from Therapy or Assessment

Patients and caregivers were to be advised verbally and in the written ICF that they had the right to withdraw from the study at any time without prejudice or loss of benefits to which they were otherwise entitled. The Investigator or Sponsor could discontinue a patient from the study in the event of an intercurrent illness, adverse event (AE), other reasons concerning the health or well-being of the patient, or in the case of lack of cooperation, noncompliance, protocol violation, or other administrative reasons. If a patient did not return for a scheduled visit, every effort was to be made to contact the patient. Regardless of the circumstance, every effort was to be made to document patient outcome, if possible. The Investigator was to inquire about the reason for withdrawal, request the caregiver return all unused investigational product (IP), and follow-up with the patient regarding any unresolved AEs.

In addition, patients who presented a QTcF>500 msec (unless due to ventricular pacing) or a QTcF interval change from the predose Baseline ECG of >60 msec at any time after randomization were withdrawn from the study. The QTcF values were assessed for clinical significance and recorded.

Patients who withdrew prior to study completion were to be asked to return to the clinic to complete the Visit 6 (end of study) assessments.

If the patient withdrew from the study, and consent was withdrawn by the caregiver and/or patient's representative for disclosure of future information, no further evaluations were to be performed, and no additional data were to be collected. The Sponsor could retain and continue to use any data that had been collected before such withdrawal of consent. Patients who withdrew from the study were not planned to be replaced.

### 3.4. Treatments

### 3.4.1. Treatments Administered

Clinical study medication was provided as hard, printed, opaque, blue, gelatin capsules (size 3) for oral administration. Each capsule of the study medication contained 1 of the following:
AVP-786-28, 28 mg of d6-DM and 4.9 mg of Q (USP, EP)
AVP-786-18, 18 mg of d6-DM and 4.9 mg of Q (USP, EP)
AVP-786 matching placebo, with the same excipients as the study medication

### 3.4.2. Identity of Investigational Product(s)

The qualitative and quantitative compositions of the 2 doses of AVP-786 and placebo are listed in Table 7.

TABLE 7
Composition of Investigational Product

| Ingredient (amounts in mg) | AVP-786-28 | AVP-786-18 | Placebo |
|---|---|---|---|
| d6-Dextromethorphan | 28.0 | 18.0 | 0 |
| hydrobromide | | | |
| Quinidine sulfate USP, | 4.9 | 4.9 | 0 |
| EP | | | |

EP = European Pharmacopoeia;
USP = United States Pharmacopoeia;
NF = National Formulary

### 3.4.3. Method of Assigning Patients to Treatment Groups

Eligible patients were randomized on Day 1 (Baseline) to receive AVP-786-18 capsules, AVP-786-28 capsules, or matching placebo capsules during Stage 1 in a double-blind manner. The randomization was stratified by NPI—Agitation/Aggression Domain score (≤6 vs >6), risk assessment for falls (normal/mild vs moderate/severe), and concomitant use of antipsychotic medications (yes vs no). Blocked randomization was used to ensure treatment balance in each stratum.

At the end of Stage 1, patients previously randomized to placebo were to be rerandomized to receive AVP-786-18 capsules, AVP-786-28 capsules, or matching placebo capsules during Stage 2 in a double-blind manner as noted above.

### 3.4.4. Selection of Doses in the Study

The doses of AVP-786 planned for this study were d6-DM 18 mg/Q 4.9 mg and d6-DM 28 mg/Q 4.9 mg, referred to as AVP-786-18 and AVP-786-28, respectively.

The 12-week duration of the double-blind treatment in this SPCD (6 weeks+6 weeks) is similar to several recently completed studies that employed this design. Given the 3-week titration period, the 6-week period duration is used to ensure exposure for at least 3 weeks to the target AVP-786 dose in each study arm believed to be sufficient for observing a treatment response based on data from prior studies. For patients assigned to the same treatment throughout the 12 weeks of Stage 1 and Stage 2, the treatment duration also allows assessment of duration of response.

### 3.4.5. Selection and Timing of Dose for Each Patient

In both Stages 1 and 2, study medication (active or placebo) was to be administered orally twice daily (1 capsule in the morning and 1 capsule in the evening approximately 12 hours apart) throughout the treatment period (without regards to food). Patients beginning active treatment in both stages were to be titrated to their randomized dose as follows:
Patients randomized to receive AVP-786-28 were to start with AVP-786-18 once a day in the morning and placebo in the evening for the first 7 days of the study. From Day 8, patients were to receive AVP-786-18 twice daily for 14 days. From Day 22, patients were to receive AVP-786-28 twice daily for the remaining 9 weeks of the study.
Patients randomized to receive AVP-786-18 were to start with AVP-786-18 once a day in the morning and placebo in the evening for the first 7 days of the study. From Day 8, patients were to receive AVP-786-18 twice daily for the remaining 11 weeks of the study.

### 3.4.6. Prior and Concomitant Therapy

Patients were not allowed to take any of the prohibited medications listed in Appendix 1 of the protocol during the study or 2 weeks or 5 half-lives, whichever was longer, before the start of dosing on Day 1. At each visit, caregivers were to be queried as to whether or not the patient had taken any concomitant medications and, if so, the Investigator was to record the medications taken and the reasons for their use. Caregivers were instructed to record concomitant use of rescue medication (lorazepam) in the diary. Concomitant use of P-glycoprotein substrates or of prodrugs whose actions are mediated by the CYP2D6-produced metabolites was to be avoided or, if necessary, carefully monitored.

### 3.4.6.1. Allowed Concomitant Medications

Drugs for the treatment of Alzheimer's disease (e.g., donepezil, rivastigmine, galantamine, memantine) were allowed when administered at stable dose for at least 3 months prior to randomization; the dose of these drugs was to remain unchanged throughout the study. If dose adjustment was necessary, the new dose and the reason for the change were to be recorded.

The use of drugs for the treatment of agitation secondary to Alzheimer's disease (e.g., atypical antipsychotics, antidepressants, buspirone) was allowed, provided the patient had been on a stable dose for at least 2 weeks before Screening and at least 1 month before randomization and throughout the study.

Concomitant use of antidepressants such as SSRIs (e.g., fluoxetine, sertraline, citalopram) and SNRIs (e.g., venlafaxine, desvenlafaxine, duloxetine) was allowed, provided the dose had been stable for at least 1 month prior to randomization and was within the Package Insert guidance for that medication. Paroxetine, a CYP2D6 substrate, was allowed provided the dose did not exceed 10 mg/day. SSRIs, SNRIs, and paroxetine had to remain stable throughout the study unless a dose reduction was deemed necessary for management of an AE.

Patients taking SSRIs or SNRIs concomitantly were monitored for serotonin syndrome which includes altered mental status, hypertension, restlessness, myoclonus, hyperthermia, hyperreflexia, diaphoresis, shivering, and tremor.

Concomitant use of hypnotics at bedtime (e.g., eszopiclone, zolpidem, zaleplon, trazodone [up to 50 mg/day]) for the nighttime treatment of insomnia was allowed, provided the dose had been stable for at least 1 month prior to randomization and remained stable throughout the study.

In addition, concomitant use of short acting benzodiazepines (e.g., midazolam, oxazepam, low dose alprazolam [up to 0.5 mg/day]) for behavioral disturbances was allowed.

All other benzodiazepines were prohibited, except for lorazepam use for short-term treatment of agitation. Patients on lorazepam prior to study entry were to be on the same treatment regimen as allowed in the study (up to 1.5 mg/day and not to exceed 3 days in a 7-day period).

### 3.4.6.2. Rescue Medication for the Symptoms of Agitation

Patients could receive oral lorazepam as rescue medication for the short-term treatment of symptoms of agitation if deemed necessary by the Investigator. Lorazepam was to be administered in a dose up to 1.5 mg/day and not to exceed 3 days in a 7-day period. Caregivers were required to record concomitant use of lorazepam in the diary and were reminded of the potential increase in the risk of falling by benzodiazepines.

### 3.4.6.3. Prohibited Concomitant Medications

A list of examples of prohibited medications was provided in Appendix 1 of the protocol. These included ketoconazole, itraconazole, voriconazole, carbonic anhydrase inhibitors, amiodarone, cimetidine, diltiazem, verapamil, protease inhibitors (e.g., saquinavir, ritonavir, atazanavir, indinavir), macrolide antibiotics (e.g., erythromycin, azithromycin, clarithromycin, dirithromycin, roxithromycin), tricyclic antidepressants (e.g., imipramine, desipramine, amitriptyline, nortriptyline), quinidine, dextromethorphan (over-the-counter and prescription), quinine, mefloquine, St. John's wort, hyperforin, rifampicin, phenytoin, carbamazepine, oxcarbazepine, phenobarbital, cyproterone, thioridazine, trifluoperazine, chlorpromazine, promazine, perphenazine, methotrimeprazine, and fluphenazine.

Monoamine oxidase inhibitors (MAOI) were prohibited throughout the study. Patients were required to allow at least 14 days after stopping study medication before starting an MAOI.

### 3.5. Efficacy and Safety Variables

### 3.5.1. Efficacy and Safety Measurements Assessed and Flow Chart

A schedule of study events is presented in Table 9. For additional details, please refer to the protocol.

TABLE 9
Schedule of Assessments

| | Visit: | | | | | | | | | |
|---|---|---|---|---|---|---|---|---|---|---|
| | | | Visit | Visit | Visit | Phone | Visit | Visit | Phone | Visit 6^{a}/ |
| | Screening^{a} | Baseline | 2^{a} | 2.1^{a} | 3^{a} | Call^{a,b} | 4^{a} | 5^{a} | Call^{a,b} | ET^{c,d} |
| | Study Day: | | | | | | | | | |
| | Day −28 | | | | | | | | | |
| | to −1 | Day 1 | Day 8 | Day 15 | Day 22 | Day 29 | Day 43 | Day 64 | Day 71 | Day 85 |
| | End of Study Week: | | | | | | | | | |
| | Week −4 | | | | | | | | | |
| Procedure | to −1 | | Week 1 | Week 2 | Week 3 | Week 4 | Week 6 | Week 9 | Week 10 | Week 12 |
| Sign informed consent | X | | | | | | | | | |
| forms | | | | | | | | | | |
| Medical history | X | | | | | | | | | |
| Review of eligibility ^{e} | X | X | | | | | | | | |
| Randomization | | X | | | | | X | | | |
| Physical and neurological | X | | | | | | X | | | X |
| examination | | | | | | | | | | |
| Vital signs and weight | X | X ^{f} | X | X | X | | X | X | | X ^{f} |
| ADCS-CGIC-Overall | | X ^{g} | | | | | X | | | X |
| CGIS-Agitation | X | X | | | | | X | | | X |
| mADCS-CGIC-Agitation | | X ^{h} | | | | | X | | | X |
| Risk assessment for falls | X | | | | | X ⁱ | | | X ^{I} | |
| (worksheet and TUG test) | | | | | | | | | | |
| ECG | X ^{j} | X ^{k} | | X | X | | X ^{k} | X | | X |
| Adverse events | | X | X | X | X | X | X | X | X | X |
| Prior and concomitant: | X | X | X | X | X | X | X | X | X | X |
| medications, nondrug | | | | | | | | | | |
| therapies, and | | | | | | | | | | |
| nonpharmacological | | | | | | | | | | |
| interventions for agitation | | | | | | | | | | |
| MMSE | X | X | | | | | X | | | X |
| GMHR | X | | | | | | | | | X |
| CMAI | X | X | X | X | X | | X | X | | X |
| NPI | X ^{l} | X | X ^{l} | X ^{l} | X | | X | X | | X |
| CSDD | X | | | | | | X | | | X |
| ZBI | | X | | | | | X | | | X |
| DEMQOL ^{m} | | X | | | | | X | | | X |
| ADAS-cog ⁿ | | X | | | | | X | | | X |
| PGIC ^{o} | | | | | | | X | | | X |
| RUD | | X | | | | | X | | | X |
| ESS | | X | | | | | X | | | X |
| S-STS | X | X | X | X | X | | X | X | | X |
| Administer AM dose of | | X | X ^{p} | X ^{p} | X | | X | X | | X |
| study medication in clinic | | | | | | | | | | |
| Chemistry, hematology, and | X ^{q} | | | | X | | X | X | | X ^{q} |
| urinalysis | | | | | | | | | | |
| Urine pregnancy test ^{r} | X | X | | | | | X | | | X |
| PK blood sample | | | | | | | X | | | X |
| CYP2D6 blood sample | | X | | | | | | | | |
| Dispense study drug & | | X | | | X | | X | X | | |
| diary card | | | | | | | | | | |
| Review/retum unused study | | | X ^{p} | X ^{p} | X | | X | X | | X |
| med & diary card | | | | | | | | | | |

Note:
Whenever possible, each patient and caregiver were to have the rating scales administered by the same raters throughout the study, for consistency of ratings. The following scales HAD TO be administered by the same rater at each visit: CMAI, NPI, mADCS-CGIC-Agitation, and CGIS-Agitation.
^{a}Study visits had a ±3-day window except Screening, Visit 2, and phone calls. Screening, Visit 2, and phone calls had a +3-day window. The Screening period could be extended after discussion with and approval by the Medical Monitor.
^{b}Phone call was to be made to patient/caregiver to collect AEs and query on concomitant medication use.
^{c}ET visit for patients who withdrew prior to study completion.
^{d}Patients who terminated early from the study or who did not roll over to the extension study (Study 15-AVP-786-303) received a safety phone call 30 days after the last dose of study medication.
^{e} For each patient, a protocol eligibility form was completed.
^{f} Weight was to be measured only at the Baseline Visit and Visit 6.
^{g} The ADCS-CGIC-Overall Baseline evaluation worksheet was to be completed to record Baseline information for assessing change at Visits 4 and 6.
^{h} The mADCS-CGIC-Agitation Baseline evaluation worksheet was to be completed to record Baseline information for assessing change at Visits 4 and 6.
ⁱ Only the TUG test was to be performed for risk assessment of falls at Visits 4 and 6.
^{j} ECG was to be performed in triplicate at the Screening Visit.
^{k} ECG was to be performed predose and postdose.
^{l} Only the Agitation/Aggression Domain of the NPI was to be performed at the Screening Visit, Visit 2, and Visit 2.1.
^{m} The proxy version was to be rated by the caregiver. The nonproxy version was to be rated only by patients with an MMSE score of ≥10 at Baseline.
ⁿ ADAS-cog was to be rated only by patients with an MMSE score of ≥10 at Baseline.
^{o} PGIC was to be rated by the caregiver.
^{p} The morning dose of study medication could be administered at home if the visit was to occur within 2 hours of dosing; the time of dosing was to be noted by the patient/caregiver. The blister card and diary card were to be returned to the patient/caregiver after reviewing for compliance.
^{q} Thyroid function tests (TSH, and reflex T3 and T4 if TSH was abnormal) were to be performed at the Screening Visit. Glycosylated hemoglobin (HbA1c) test was to be performed at the Screening Visit and Visit 6.
^{r} Urine pregnancy test was to be performed for females of childbearing potential only.

### 3.5.1.1. Efficacy Endpoints

The efficacy endpoints included validated scales and questionnaires to assess changes in behaviors associated with agitation, depression, cognitive dysfunction, quality of life (QOL), and caregiver stress. A statistical gatekeeping procedure was applied to the primary (CMAI Total score) and key secondary efficacy endpoints (Modified Alzheimer's Disease Cooperative Study-Clinical Global Impression of Change [mADCS-CGIC]-Agitation score) to control the overall type 1 error at a 2-sided α=0.05 significance level

### 3.5.1.1.1. Primary Efficacy Assessments

The primary efficacy endpoint was the change from Baseline to Week 6 (Stage 1), from Week 6 to Week 12 (Stage 2), and from Baseline to Week 12 (12-week Parallel Group) in the composite CMAI scores (CMAI Total score). The CMAI was used to assess the frequency of manifestations of agitated behaviors in elderly persons. It consists of 29 agitated behaviors that are further categorized into distinct agitation syndromes, also known as CMAI factors of agitation. These distinct agitation syndromes include: F1-Aggressive Behavior, F2—Physically Nonaggressive Behavior, and F3—Verbally Agitated Behavior, and are secondary efficacy endpoints. Scores for the 3 dimensions Factor 1, Factor 2, and Factor 3 were derived based on the factor structure described by Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998 and described elsewhere herein.

Each of the 29 items is rated on a 7-point scale of frequency (1=never, 2=less than once a week but still occurring, 3=once or twice a week, 4=several times a week, 5=once or twice a day, 6=several times a day, 7=several times an hour). The ratings are based on the 2 weeks preceding assessment of CMAI; a decrease in CMAI scores indicates improvement in the frequency of agitated behaviors. The CMAI Total score is calculated as the sum of ratings for all 29 items and ranges from 29 to 203.

The CMAI was assessed at Screening, Day 1 (Baseline), and at Weeks 1, 2, 3, and 6 during Stage 1 and at Weeks 9 and 12 during Stage 2 (Table 9); the Stage 2 Baseline was the last CMAI assessment prior to Stage 2 rerandomization.

### 3.5.1.1.2. Secondary Efficacy Assessments

The key secondary efficacy endpoint was the Modified Alzheimer's Disease Cooperative Study-Clinical Global Impression of Change-Agitation (mADCS-CGIC-Agitation), which was assessed at Day 1 (Baseline), Week 6, and Week 12:
mADCS-CGIC-Agitation: The mADCS-CGIC-Agitation is a modification of the standard Alzheimer's Disease Cooperative Study-Clinical Global Impression of Change (ADCS-CGIC) instrument to better assess aspects relevant to studying agitation in Alzheimer's disease. It contains questions related to agitation and an assessment of the Clinician's Impression of Change focused specifically on agitation. It was originally designed for the Citalopram Study for Agitation in Alzheimer's Disease (CitAD) and utilizes a semi-structured interview of both patient and caregiver to determine a Baseline level of severity for agitation. Subsequent evaluations assess for change from Baseline and utilize the semi-structured agitation interview of both patient and caregiver.

Additional secondary efficacy endpoints, assessed at the timepoints indicated in Table 9, included the following:
NPI—Agitation/Aggression Domain score and Caregiver Distress score: This is a validated clinical instrument for evaluating psychopathology in a variety of disease settings, including dementia. The NPI is a retrospective caregiver-informant interview covering 12 neuropsychiatric symptom domains: delusions, hallucinations, agitation/aggression, depression/dysphoria, anxiety, elation/euphoria, apathy/indifference, disinhibition, irritability/lability, aberrant motor behavior, sleep and nighttime behavioral disorders, and appetite/eating disorders. The scripted NPI interview includes a compound Screening question for each symptom domain, followed by a list of interrogatives about domain-specific behaviors that is administered when a positive response to a Screening question is elicited. Neuropsychiatric manifestations within a domain are collectively rated by the caregiver in terms of both frequency (1 to 4) and severity (1 to 3), yielding a composite symptom domain score (frequency×severity). Frequency and severity rating scales have defined anchor points to enhance the reliability of caregiver responses. Caregiver Distress is rated for each positive neuropsychiatric symptom domain on a scale anchored by scores of 0 (not distressing at all) to 5 (extremely distressing). The NPI domains are generally evaluated for behaviors within the preceding 4 weeks but can be modified according to the needs of the study; in this study, the recall period was 2 weeks for all the visits. The NPI nursing home version (NPI-NH) was used for patients from in-patient or assisted-living facilities. The questions in the NPI-NH were rephrased for professional caregivers who might not know the patients prior to the onset of illness; however, the overall instrument domains and scoring was identical to the NPI except for the Caregiver Distress section, which was replaced with occupational disruptiveness in the NPI-NH version. The Agitation/Aggression Domain score in the NPI was assessed as part of the NPI Total score.
NPI—Aberrant Motor Behavior Domain (see above).
CGIS-Agitation score (CGIS-Agitation): This is an observer-rated scale that measures illness severity and is one of the most widely used brief assessment tools in psychiatry research. The Early Clinical Drug Evaluation Unit (ECDEU) version of the CGIS is the most widely used format of this validated tool, and it asks that the clinician rate the patient relative to their past experience with other patients with the same diagnosis, with or without collateral information. The CGIS has proved to be a robust measure of efficacy in many clinical drug trials, and it is easy and quick to administer, provided that the clinician knows the patient well. Reliability and validity of CGI have been tested in multiple studies, including patients with dementia, schizophrenia, and affective disorders. Overall, CGI showed high correlation (r: ˜90%) with other assessment instruments and it has also shown positive significant relationships and concurrent validity with other clinician's rating. In addition, the scale has good sensitivity to change over time. The CGIS score is a 7-point (1-7) scale (1=normal, not at all ill; 7=among the most extremely ill patients) and assesses severity of agitation in this study.
Alzheimer's Disease Cooperative Study-Clinical Global Impression of Change for Overall Clinical Status (ADCS-CGIC-Overall): This scale is to provide a means to reliably assess change from a Baseline level of global function within the timeframe of a clinical trial. Unlike a targeted symptom scale, the ADCS-CGIC-Overall takes into account a patient's overall function in the cognitive, behavioral, and functional activity domains. Relying on information gathered through a semi-structured interview of the patient and caregiver, the ADCS-CGIC-Overall focuses on clinician's observations of change in the patient's cognitive, functional, and behavioral performance since the beginning of a trial. Once the Baseline level of severity is established, the change score at the follow-up visits is based on information gathered from the patient and caregiver interviews. The ADCS-CGIC-Overall is rated as: marked improvement, moderate improvement, minimal improvement, no change, minimal worsening, moderate worsening, or marked worsening.
Zarit Burden Interview (ZBI): This is a 22-item scale used to assess the impact of patient's disabilities on the caregiver's life. It is designed to reflect the burden experienced by caregivers of dementia patients and can either be completed by the caregiver or administered as an interview. It is the most commonly used scale for measuring burden in caregivers' of patients with dementia and also other illnesses. The ZBI has been shown to have high internal-reliability with an estimated Cronbach's alpha at 0.88 and 0.91, and test-retest reliability at 0.71. Validity has been estimated by correlating the total score with a single global rating of burden (r=0.71). For each item of the scale, the caregiver has to indicate how often they felt that way (never, rarely, sometimes, quite frequently, or nearly always). The score ranges from 0 to 88 and is determined by adding the numbered responses of the individual items. Higher scores indicate greater Caregiver Distress.
NPI—Irritability/Lability Domain (see above).
NPI Total score (see above).
Patient Global Impression of Change (PGIC): This is a 7 point (1-7) scale used to assess treatment response, and it is rated as: very much improved, much improved, minimally improved, no change, minimally worse, much worse, or very much worse.
Dementia Quality of Life (DEMQOL): This is a scale used to evaluate health-related QOL in patients with dementia and their caregivers. There are 2 versions of the DEMQOL, a 28-item version (rated by patient) and a 31-item version (DEMQOL-proxy, rated by caregiver). Both the 28-item and 31 item version are recommended to be used for evaluating patients (and their caregivers) with mild to moderate dementia (MMSE≥10). For patients with severe dementia, only the DEMQOL-proxy (administered to caregiver) is used.
Alzheimer's Disease Assessment Scale-cognitive subscale (ADAS-cog): The ADAS was designed to evaluate the cognitive and non-cognitive behavioral dysfunction characteristics of patients with Alzheimer's disease. The cognitive subscale (ADAS-cog) consists of 11 subsets related to memory, praxis, and language. The ADAS-cog takes about 30 to 45 minutes to complete. The ADAS-cog was assessed for patients with an MMSE score of ≥10 at the Baseline Visit.
CSDD: This scale was specifically developed to assess signs and symptoms of major depression in patients with dementia. Because some of these patients may give unreliable reports, the CSDD uses a comprehensive interviewing approach that derives information from the patient and the caregiver. Information is elicited through 2 semi-structured interviews, an interview with a caregiver and an interview with the patient. The interviews focus on depressive symptoms and signs occurring during the week preceding the assessment. Each item is rated for severity on a scale of 0-2 (0=absent, 1=mild or intermittent, 2=severe). The item scores are added. Scores above 10 indicate a probable major depression, scores above 18 indicate a definite major depression and scores below 6 as a rule are associated with absence of significant depressive symptoms.
Resource Utilization in Dementia (RUD): The RUD is used to calculate healthcare costs associated with dementia. It evaluates dementia patients' utilization of formal and informal healthcare resources, including hospitalizations and doctor visits, living assistance, and time spent by nonprofessional caregivers. Within the context of clinical trials, the RUD is often used to determine the cost effectiveness of new pharmaceutical treatments. The RUD is administered as a semi-structured interview with the patient's primary caregiver and contains 2 sections; one focusing on caregiver impact (loss of work and leisure time incurred by caregiver) and the other focusing on the patient's use of healthcare resources. The total healthcare costs associated with the patient's dementia can be estimated by multiplying the number of units used (e.g., hours of caregiver time, visits to doctors, nights in accommodation) by the corresponding unit price vector.
General Medical Health Rating (GMHR): This is a global clinical rating for medical health, designed to quantify in a single number (1 to 4) the severity of general comorbidity in a patient with dementia. A rating of 1=poor, 2=fair, 3=good and 4=excellent to very good.

### 3.5.1.2. Safety Endpoints

The safety endpoints evaluated were treatment-emergent adverse events (TEAEs), clinical laboratory results, vital signs (including blood pressure), ECGs, S-STS, MMSE, Timed Up and Go (TUG) Test, and the Epworth Sleepiness Scale (ESS).

### 3.5.1.2.1. Safety Assessments

### 3.5.1.2.1.1. Adverse Events

Caregivers were queried regarding TEAEs at each clinic visit after the Screening Visit (Table 9) and at the safety phone calls at Days 29 and 71. All reported TEAEs were assessed and recorded. Any AE newly reported after receiving the last dose of study medication was followed up until 30 days.

The severity of each AE was graded on a 3-point scale (mild, moderate, or severe) and reported in detail as indicated on the electronic case report form (eCRF). The relationship of each AE to study medication was determined by the Investigator as not related, unlikely related, possibly related, or related.

### 3.5.1.2.1.2. Physical and Neurological Examination

Physical and neurological examinations were performed at Screening (Day −28 to Day −1), Day 43 (Visit 4), and Day 85 (Visit 6). The physical examination included assessments of head, eyes, ears, nose, throat, lymph nodes, skin, extremities, respiratory, gastrointestinal, musculoskeletal, cardiovascular, and nervous systems. The neurological examination included assessments of mental status, cranial nerves, motor system, reflexes, coordination, gait and station, and sensory system. The physical and neurological examinations were performed by the same person each time, whenever possible.

Physical and neurological examination abnormalities determined by the Investigator to be clinically significant at Screening were recorded as medical history. Any clinically significant changes in physical and neurological examination findings from the Screening examination were recorded as AEs.

### 3.5.1.2.1.3. Clinical Laboratory Tests

The following clinical laboratory assessments were performed at the timepoints indicated in Table 9:
Blood chemistry (calcium, magnesium, phosphorus, glucose, sodium, potassium, chloride, carbon dioxide, blood urea nitrogen [BUN], serum creatinine, uric acid, albumin, total bilirubin, alkaline phosphatase, lactate dehydrogenase [LDH], aspartate aminotransferase [AST], alanine aminotransferase [ALT], creatine kinase [CK], gamma-glutamyl transferase [GGT], triglycerides, total protein, and total cholesterol)
Hematology (red blood cell [RBC] count, hemoglobin, hematocrit, white blood cell [WBC] count, neutrophils, bands, lymphocytes, monocytes, eosinophils, basophils, platelet count, and morphology)
Urinalysis (pH, specific gravity, protein, glucose, ketones, blood, leucocyte esterase, nitrates, and microscopic appearance)
Thyroid function tests (thyroid-stimulating hormone [TSH], and reflex triiodothyronine [T3] and thyroxine [T4] if TSH is abnormal) at Screening Visit only
Glycosylated hemoglobin (HbA1c) test at the Screening Visit and Visit 6 only

Urine pregnancy tests were performed for females of childbearing potential at the timepoints indicated in Table 9.

All female patients of childbearing potential were instructed to use appropriate birth control methods for up to 4 weeks following the last dose of study medication.

Any clinically significant laboratory test result could have required a repeat if requested by the Medical Monitor.

### 3.5.1.2.1.4. Electrocardiograms

A resting 12-lead ECG was performed at the timepoints indicated in Table 9. At Screening, ECG was performed in triplicate. At Baseline (Day 1) and Day 43 (Visit 4), 2 ECGs were performed; one prior to study medication dosing and one 2 to 3 hours after dosing. ECG equipment was provided by the central reader. ECG data were recorded at the study center and included general findings, heart rate (beats/minute), QRS complex, and PR and QTc intervals (milliseconds). Results were provided by the central reader to the Investigators within 24 hours.

### 3.5.1.2.1.5. Sheehan Suicidality Tracking Scale

The S-STS is a prospective scale that assesses treatment-emergent suicidal thoughts and behaviors and was assessed at the timepoints indicated in Table 9. Any change in the S-STS score indicating the presence of suicidality was evaluated by the Investigator and reported to the Medical Monitor.

### 3.5.1.2.1.6. Mini Mental State Examination

The MMSE is a brief 30-point questionnaire test that is used to screen for cognitive impairment and was assessed at the timepoints indicated in Table 9.

### 3.5.1.2.1.7. Timed Up and Go Test

The TUG test measures the time (in seconds) taken for an individual to stand up from a standard armchair, walk 3 meters, turn, walk back to the chair and sit down; the test was assessed at the timepoints indicated in Table 9.

### 3.5.1.2.1.8. Epworth Sleepiness Scale

The ESS is an 8-item questionnaire that is used to measure sleepiness by rating the probability of falling asleep on 8 different situations that most people engage in during the day; the test was assessed at the timepoints indicated in Table 9.

### 3.5.1.3. Pharmacokinetic Assessments

At Day 43 (Visit 4) and Day 85 (Visit 6), patients had a blood sample collected between 0 and 3 hours after the morning dose of study medication for analysis of plasma levels of d6-DM, d6-DM metabolites, and Q. The time when the patient was administered the dose of study medication and the time of the blood draw were recorded. Plasma samples were separated by centrifugation and frozen at −20° C. until assayed at the analytical unit.

### 3.5.2. Primary Efficacy Variable(s)

The primary efficacy endpoints and assessments are described elsewhere herein.

### 3.5.3. Drug Concentration Measurements

Pharmacokinetic assessments performed in this study are described elsewhere herein.

### 3.6. Data Quality Assurance

### 3.6.1. Laboratory Data

Each individual site laboratory was required to collect hematology, blood chemistry, and urinalysis samples at Screening (Day −28 to Day −1), and Visits 3 to 6 (Day 22, Day 43, Day 64, and Day 85) for safety analysis. Instructions for specimen evaluation and transport to a central laboratory were to be provided at the time of study initiation. Blood samples were also required to be taken for CYP2D6 genotyping at Baseline (Day 1) and for PK analysis on Visits 4 and 6 (Day 43 and Day 85).

### 3.7. Statistical Methods Planned in the Protocol and Determination of Sample Size

### 3.7.1.1. Analysis Populations

There were 4 analysis populations: mITT, intent-to-treat (ITT), Safety, and the 12-week Parallel Group, which are defined below.

3.7.1.1.1. mITT Population

The mITT population was used for all efficacy and health outcome analyses. Due to the study design, the patients included in the mITT population were determined separately for Stage 1 and Stage 2, although the Stage 2 group was a subset of the Stage 1 group. Patients were included in the treatment group to which they were randomized regardless of treatment received. The mITT population is defined below for each stage:
Stage 1: All patients who were randomized in Stage 1 and had at least 1 postbaseline efficacy assessment.
Stage 2: All patients who were rerandomized in Stage 2 and had at least 1 efficacy assessment in Stage 2 (after Week 6).

### 3.7.1.1.2. ITT Population

The ITT population was used for sensitivity analyses. Patients were included in the treatment group to which they were randomized regardless of treatment received. The ITT population was defined below:
Stage 1: All patients who were randomized in Stage 1
Stage 2: All patients who were rerandomized in Stage 2

### 3.7.1.1.3. Safety Population

The Safety Population includes all patients who received at least 1 dose of study medication. The Safety Population was used for all analyses of safety data. Patients were included in the treatment group based on the actual treatment received.

3.7.1.1.4. 12-week Parallel Group Population

The 12-week Parallel Group Population is the cohort of patients who were randomized to the same treatment in both stages (Stages 1 and 2). Since all Stage 1 placebo patients, including those who dropped out in Stage 1, were rerandomized and assigned a treatment group in Stage 2, this population is similar to a group in a 12-week, randomized, parallel-group design with a total planned sample size of 254 (⅔ of the original sample size 380) and treatment ratio of 3:3:2 (active:active:placebo).

This population is intended to be used to evaluate efficacy and safety over 12 weeks of treatment comparing AVP-786-28, AVP-786-18, and placebo in a parallel-group design setting. It includes patients from Treatment Segments A, D, and G (FIG. 1) in placebo; B and J (FIG. 1) in AVP-786-28; and C and K (FIG. 1) in AVP-786-18.
12-week Parallel Group Population: patients in the 12-week Parallel Group Population who have at least 1 postbaseline efficacy assessment.
Safety 12-week Parallel Group Population: patients in the 12-week Parallel Group Population who received at least 1 dose of study medication.

### 3.7.1.2. Efficacy

### 3.7.1.2.1. Primary Efficacy Endpoint Analysis Methods

### 3.7.1.2.1.1. Primary Analysis

For the primary efficacy analysis, the treatment effect was estimated using a likelihood-based mixed model repeated measures (MMRM) on observed data in each stage separately. The treatment effect estimates were combined in a weighted test statistic with a weight of 0.6 for Stage 1 and a weight of 0.4 for Stage 2. The Stage 1 model included terms for treatment, visit, treatment-by-visit interaction, Baseline CMAI Total score, Baseline-by-visit interaction, Baseline NPI—Agitation/Aggression (≤6 vs >6), risk assessment for falls (normal/mild vs moderate/severe), and concomitant use of antipsychotic medications (yes vs no). The Stage 2 model included terms for treatment, visit, treatment-by-visit interaction, and Stage 2 Baseline. An unstructured covariance matrix was planned for both models. If there were convergence issues, then the following covariance structures other than the unstructured were to be used in the order of 1) autoregressive of order 1, 2) compound symmetry (CS) and the covariance structure converging to the best fit would be used as the primary analysis. Under the missing at random assumption, MMRM provides an unbiased estimate of treatment effect for the treatment period.

Model estimates (treatment difference and its 95% confidence interval [CI]) are reported for each stage.

For rerandomization (as the stratification variable) and analysis in Stage 2, Placebo Responders and Nonresponders in Stage 1 were defined as follows:
Placebo Responders were patients randomized to placebo in Stage 1 whose Clinical Global Impression of Severity of Illness scale for Agitation (CGIS-Agitation) score was ≤3 at Visit 4 (Day 43) and NPI—Agitation/Aggression Domain score has decreased by ≥25% from Baseline.
Placebo Nonresponders were patients randomized to placebo in Stage 1 who do not meet the criteria for Responder as defined above.

### 3.7.1.3. Safety

Descriptive statistics and by-patient listings are presented for safety assessments, including TEAEs, clinical laboratory assessments, ECGs, vital signs, physical and neurological examinations, S-STS, MMSE, TUG test, and ESS. All safety analyses will be completed on the Safety Population.

In general, categorical safety analyses (e.g., TEAEs) are displayed using the following treatment groups:
1. Placebo: patients who received placebo for the entire duration of the study (Treatment Segments D and G from the SPCD schematic (FIG. 1), including data from Treatment Segment A during Stage 1. Note that patients randomized to placebo/AVP-786 but dropped out in Stage 1 are not included in this population. Instead, their data are summarized under the corresponding placebo/AVP-786 treatment group.
2. AVP-786-28: patients who received AVP-786-28 for the entire duration of the study (B and J in FIG. 1.
3. AVP-786-18: patients who received AVP-786-18 for the entire duration of the study (C and K in FIG. 1.
4. Placebo/AVP-786-28: patients who received placebo during Stage 1 and AVP-786-28 during Stage 2 (E and H in FIG. 1), including those who received placebo and dropped out in Stage 1. This group is further divided into data that occurred on placebo (Stage 1) and data that occurred on AVP-786-28 (Stage 2).
5. Placebo/AVP-786-18: patients who received placebo during Stage 1 and AVP-786-18 during Stage 2 (F and I in FIG. 1) including those who received placebo and dropped out in Stage 1. This group is further divided into data that occurred on placebo (Stage 1) and data that occurred on AVP-786-18 (Stage 2).
6. All Placebo: patients who received placebo at any time during the study, including all patients in Segment A during Stage 1 and all patients in Segments D and G during Stage 2 (FIG. 1). For patients who received both placebo and active treatment, only data from the placebo treatment period are included in the All Placebo group.
7. All AVP-786-28: patients who received AVP-786-28 at any time during the study, including all patients in Segment B during Stage 1 and all patients in Segments J, E, and H during Stage 2 (FIG. 1). For patients who received both placebo and AVP-786-28 treatment, only data from the AVP-786-28 treatment period are included in the All AVP-786-28 group.
8. All AVP-786-18: patients who received AVP-786-18 at any time during the study, including all patients in Segment C during Stage 1 and all patients in Segments K, F, and I during Stage 2 (FIG. 1). For patients who received both placebo and AVP-786-18 treatment, only data from the AVP-786-18 treatment period are included in the All AVP-786-18 group.

The placebo, AVP-786-18, and AVP-786-28 groups summarize the safety information for the 12-week Parallel Group Safety Population, which received 12 weeks of treatment exposure. It is what would be summarized if the study had been a 12-week parallel-group design.

The All Placebo, All AVP-786-18, and All AVP-786-28 groups summarize the safety information for their corresponding treatment group under 6 weeks or 12 weeks of treatment exposure in either Stage 1, Stage 2, or both.

For quantitative summaries (e.g., ECGs, laboratory tests), the placebo and AVP-786 groups were not included.

### 4. Study Patients

### 4.1. Disposition of Patients

Overall Patient Disposition (All Patients)

Of the 387 patients randomized to treatment, most patients completed the study (89.9%). A total of 39 patients (10.1%) discontinued from the study early. The most common reasons for early discontinuation overall were TEAEs (3.9%), withdrawal by subject (2.1%), and study subject withdrawal by parent or guardian (1.6%).

Patient Disposition in Stage 1 (mITT)

Of the 387 patients randomized to treatment in Stage 1, 382 patients had at least 1 postbaseline efficacy assessment and were included in the mITT population (Table 11), comprising 191, 94, and 97 patients in the placebo, AVP-786-18, and AVP-786-28 groups, respectively. Most patients completed Stage 1 (364 [95.3%]). A total of 18 (4.7%) patients discontinued treatment before completing Stage 1, comprising 9 (4.7%), 7 (7.4%), and 2 (2.1%) patients in the placebo, AVP-786-18, and AVP-786-28 groups, respectively. The most common reason for discontinuation from Stage 1 was due to TEAEs (2.6% overall). Patients treated with AVP-786-18 had a higher rate of discontinuation due to TEAEs (5.3%) compared with placebo (2.1%) and AVP-786-28 (1.0%).

Patient Disposition in Stage 2 (mITT)

For the placebo group, a total of 182 patients (95.3%) completed Stage 1 and were rerandomized into Stage 2. Of these, 177 placebo patients were included in the Stage 2 mITT population; 58, 59, and 60 patients were rerandomized into the placebo/placebo, placebo/AVP-786-18, and placebo/AVP-786-28 groups, respectively (Table 11). A total of 7 patients discontinued before completing Stage 2, comprising 2 (3.4%), 0, and 5 (8.3%) patients in the placebo/placebo, placebo/AVP-786-18, and placebo/AVP-786-28 groups, respectively.

Of the 177 placebo patients included in the Stage 2 mITT population, there were 125 Placebo Nonresponders; 40, 41, and 44 patients were rerandomized into the placebo/placebo, placebo/AVP-786-18, and placebo/AVP-786-28 groups, respectively (Table 11). A total of 6 patients discontinued before completing Stage 2, comprising 2 (5.0%), 0, and 4 (9.1%) patients in the placebo/placebo, placebo/AVP-786-18, and placebo/AVP-786-28 groups, respectively.

Of the 177 placebo patients included in the Stage 2 mITT population, there were 52 Placebo Responders; 18, 18, and 16 patients were rerandomized into the placebo/placebo, placebo/AVP-786-18, and placebo/AVP-786-28 groups, respectively (Table 11). One patient discontinued before completing Stage 2 (6.3% in the placebo/AVP-786-28 group).

Patient Disposition for 12-Week Parallel Group (mITT)

A total of 253 patients received the same treatment for the entire duration of the study (12-week Parallel Group), comprising 62, 94, and 97 patients in the placebo, AVP-786-18, and AVP-786-28 groups, respectively. A total of 19 (7.5%) patients discontinued before completing the study, comprising 6 (9.7%), 8 (8.5%), and 5 (5.2%) patients in the placebo, AVP-786-18, and AVP-786-28 groups, respectively. Discontinuation due to TEAEs was the most common reason for discontinuation overall (3.6%); patients treated with AVP-786-18 had a higher rate of discontinuation due to TEAEs (6.4%) compared with placebo (3.2%) and AVP-786-28 (1.0%).

TABLE 10
Overall Patient Disposition (All Patients)

| | | | | | | | Placebo/ | | Placebo/ | | All | | All | | All | |
|---|---|---|---|---|---|---|---|---|---|---|---|---|---|---|---|---|
| | Placebo | | AVP-786-18 | | AVP-786-28 | | AVP-786-18 | | AVP-786-28 | | AVP-786-18 | | AVP-786-28 | | Patients | |
| | (N = 63) | | (N = 96) | | (N = 97) | | (N = 65) | | (N = 66) | | (N = 161) | | (N = 163) | | (N = 695) | |
| | n (%) | | n (%) | | n (%) | | n (%) | | n (%) | | n (%) | | n (%) | | n (%) | |
| Patients Screened | | | | | | | | | | | | | | | 695 | |
| Screen Failures | | | | | | | | | | | | | | | 308 | (44.3) |
| Adverse Event | | | | | | | | | | | | | | | 3 | (0.4) |
| Inclusion/Exclusion | | | | | | | | | | | | | | | 237 | (34.1) |
| Criterion Met | | | | | | | | | | | | | | | | |
| Lost to Follow-up | | | | | | | | | | | | | | | 6 | (0.9) |
| Withdrew Consent | | | | | | | | | | | | | | | 37 | (5.3) |
| Other | | | | | | | | | | | | | | | 25 | (3.6) |
| Patients Randomized | 63 | | 96 | | 97 | | 65 | | 66 | | 161 | | 163 | | 387 | |
| Randomized Patients who | 0 | | 1 | (1.0) | 0 | | 0 | | 0 | | 1 | (0.6) | 0 | | 1 | (0.3) |
| did not Receive Study | | | | | | | | | | | | | | | | |
| Medications | | | | | | | | | | | | | | | | |
| Completed Study | 56 | (88.9) | 86 | (89.6) | 92 | (94.8) | 59 | (90.8) | 55 | (83.3) | 145 | (90.1) | 147 | (90.2) | 348 | (89.9) |
| Patients who Discontinued | 7 | (11.1) | 10 | (10.4) | 5 | (5.2) | 6 | (9.2) | 11 | (16.7) | 16 | (9.9) | 16 | (9.8) | 39 | (10.1) |
| from Study | | | | | | | | | | | | | | | | |
| Adverse Event | 2 | (3.2) | 6 | (6.3) | 1 | (1.0) | 1 | (1.5) | 5 | (7.6) | 7 | (4.3) | 6 | (3.7) | 15 | (3.9) |

| | | | | | | | | | | | | | |
|---|---|---|---|---|---|---|---|---|---|---|---|---|---|
| Death | 1 | (1.6) | 1 | (1.0) | 0 | 2 | (3.1) | 0 | 3 | (1.9) | 0 | 4 | (1.0) |

| Lack of Efficacy | 1 | (1.6) | 0 | 0 | 0 | | 0 | 0 | | 0 | 1 | (0.3) |
|---|---|---|---|---|---|---|---|---|---|---|---|---|
| Lost to Follow-up | 1 | (1.6) | 0 | 0 | 1 | (1.5) | 0 | 1 | (0.6) | 0 | 2 | (0.5) |

| Protocol Deviation | 1 | (1.6) | 0 | 0 | | 0 | 0 | | 0 | 0 | | 1 | (0.3) |
|---|---|---|---|---|---|---|---|---|---|---|---|---|---|
| Study Subject Withdrawal | 1 | (1.6) | 0 | 1 | (1.0) | 0 | 4 | (6.1) | 0 | 5 | (3.1) | 6 | (1.6) |
| by Parent or Guardian | | | | | | | | | | | | | |

| | | | | | | | | | | | | | | | |
|---|---|---|---|---|---|---|---|---|---|---|---|---|---|---|---|
| Withdrawal by Subject | 0 | 2 | (2.1) | 3 | (3.1) | 2 | (3.1) | 1 | (1.5) | 4 | (2.5) | 4 | (2.5) | 8 | (2.1) |

| | | | | | | | | | | | | | |
|---|---|---|---|---|---|---|---|---|---|---|---|---|---|
| Other | 0 | 1 | (1.0) | 0 | 0 | 1 | (1.5) | 1 | (0.6) | 1 | (0.6) | 2 | (0.5) |

Note:
Denominators for screen failures and reasons for screen failures are the number of patients screened. Denominators for all other categories are the number of patients randomized in each group.
No subjects discontinued because of noncompliance with study drug, physician decision, pregnancy, study terminated by Sponsor, or trial site terminated by Sponsor.

TABLE 11
Patient Disposition by Stage (mITT Population)

| | Placebo | AVP-786-18 | AVP-786-28 | All Patients |
|---|---|---|---|---|
| Patient Status/Disposition | n (%) | n (%) | n (%) | n (%) |
| Stage 1 | | | | |
| Stage 1 mITT Population | N = 191 | N = 94 | N = 97 | N = 382 |
| Completed Stage 1 | 182. (95.3) | 87 (92.6) | 95 (97.9) | 364 (95.3) |
| Discontinued from Study in Stage 1 | 9 (4.7) | 7 (7.4) | 2 (2.1) | 18 (4.7) |
| Reason for discontinuation ^{a} | | | | |
| Adverse Event | 4 (2.1) | 5 (5.3) | 1 (1.0) | 10 (2.6) |
| Lost to Follow-up | 1 (0.5) | 0 | 0 | 1 (0.3) |
| Study Subject Withdrawal by Parent or Guardian | 2 (1.0) | 0 | 0 | 2 (0.5) |
| Withdrawal by Subject | 1 (0.5) | 2 (2.1) | 1 (1.0) | 4 (1.0) |
| Other | 1 (0.5) | 0 | 0 | 1 (0.3) |

| | Placebo | AVP-786-18 | AVP-786-28 |
|---|---|---|---|
| Patient Status/Disposition | n (%) | n (%) | n (%) |
| Stage 2 | | | |
| Placebo Nonresponders | N = 40 | N = 4l | N = 44 |
| Completed Stage 2 | 38 (95.0) | 41 (100) | 40 (90.9) |
| Discontinued from Study in Stage 2 | 2 (5.0) | 0 | 4 (9.1) |
| Reason for discontinuation ^{b} | | | |
| Adverse Event | 0 | 0 | 3 (6.8) |
| Lack of Efficacy | 1 (2.5) | 0 | 0 |
| Prolocol Deviation | 1 (2.5) | 0 | 0 |
| Study Subject Withdrawal by Parent or Guardian | 0 | 0 | 1 (2.3) |

| | Placebo | AVP-786-18 | A VP-786-28 | All Patients |
|---|---|---|---|---|
| Patient Status/Disposition | n (%) | n (%) | n (%) | rt (%) |
| Placebo Responders | N =18 | N = 18 | N = 16 | |
| Completed Stage 2 | 18 (100) | 18 (100) | 15 (93.8) | |
| Discontinued from Study in Stage 2 | 0 | 0 | 1 (6.3) | |
| Reason for discontinuation ^{b} | | | | |
| Study Subject Withdrawal by Parent or Guardian | 0 | 0 | 1 (6.3) | |

| | Placebo | AVP-786-18 | AVP-786-28 |
|---|---|---|---|
| Patient Status/Disposition | n (%) | n (%) | n (%) |
| Placebo Responders/Nonresponders Combined | N = 58 | N = 59 | N = 60 |
| Completed Stage 2 | 56 (96.6) | 59 (100) | 55 (91.7) |
| Discontinued from Study in Stage 2 | 2 (3.4) | 0 | 5 (8.3) |
| Reason for discontinuation ^{b} | | | |
| Adverse Event | 0 | 0 | 3 (5.0) |
| Lack of Efficacy | 1 (1.7) | 0 | 0 |
| Protocol Deviation | 1 (1.7) | 0 | 0 |
| Study Subject Withdrawal by Parent or Guardian | 0 | 0 | 2 (3.3) |
| Other | 0 | 0 | 0 |

mITT = modified intent-to-treat
^{a} No patients discontinued Stage 1 because of death, lack of efficacy, noncompliance with study drug, physician decision, pregnancy, protocol deviation, study terminated by Sponsor, or trial site terminated by Sponsor.
b No patients discontinued Stage 2 because of death, lost to follow-up, noncompliance with study drug, physician decision, pregnancy, study terminated by Sponsor, trial site terminated by Sponsor, withdrawal by subject, or other.

### 5. Efficacy Evaluation

### 5.1. Data Sets Analyzed

Analysis sets are summarized in Table 14. For Stage 1, all 387 randomized patients were included in the ITT population, and 382 randomized patients were included in the mITT population. A total of 5 patients were excluded from the mITT population, all due to the lack of a postbaseline efficacy assessment (3 and 2 patients randomized to placebo and AVP-786-18, respectively). No patients randomized to AVP-786-28 were excluded from the mITT population.

For the 12-week Parallel Group (patients who received the same treatment for the entire duration of the study), 253 randomized patients were included in the mITT population. A total of 255 randomized patients were included in the Safety Population.

TABLE 14
Summary of Analysis Populations and Stage 2 Subsets (All Randomized Patients)

| | Stage 1 | | | | Stage 2 | | | |
|---|---|---|---|---|---|---|---|---|
| | | | | All | | | | All |
| Analysis Population/Subset, n | Placebo | AVP-786-18 | AVP-786-28 | Patients | Placebo | AVP-786-18 | AVP-786-28 | Patients |
| Randomized | 194 | 96 | 97 | 387 | | | | |
| Study Populations | | | | | | | | |
| Intent-to-Treat (ITT) | 194 | 96 | 97 | 387 | | | | |
| Modified Intent-to-Treat (mITT) | 191 | 94 | 97 | 382 | | | | |
| Safety | 194 | 95 | 97 | 386 | | | | |
| Stage 2 mITT Subsets | | | | | | | | |
| Placebo Nonresponders | | | | | 40 | 41 | 44 | 125 |
| Placebo Responders | | | | | 18 | 18 | 16 | 52 |
| Placebo Nonresponders + Responders | | | | | 58 | 59 | 60 | 177 |

Both Stages

| | | AVP-786 | AVP-786 | All |
|---|---|---|---|---|
| | Placebo | 18 mg | 28 mg | Patients |
| mITT 12-week Parallel Group | 62 | 94 | 97 | 253 |
| Safety 12-week Parallel Group | 63 | 95 | 97 | 255 |

Note:
Under 12-week Parallel Group, placebo and AVP-786 represent placebo and AVP-786/AVP-786 for their corresponding Stage 1/Stage 2 treatments, respectively. Safety 12-week Parallel Group Population is based on patients in the 12-week Parallel Group Population who received at least one dose of study medication.

For the discussion of efficacy results, groups are named as follows:
Stage 1
   Placebo: All patients randomized to placebo for Stage 1 and included in the mITT population (A in FIG. 1; N=191)
   AVP-786-18: All patients randomized to AVP-786-18 for Stage 1 and included in the mITT population (C in FIG. 1; N=94)
   AVP-786-28: All patients randomized to AVP-786-28 for Stage 1 and included in the mITT population (B in FIG. 1; N=97)
Stage 2 Placebo Nonresponders
   Placebo/Placebo: Placebo Nonresponders randomized to placebo for Stage 2 and included in the mITT population (D FIG. 1; N=40)
   Placebo/AVP-786-18: Placebo Nonresponders randomized to AVP-786-18 for Stage 2 and included in the mITT population (F in FIG. 1; N=41)
   Placebo/AVP-786-28: Placebo Nonresponders randomized to AVP-786-28 for Stage 2 and included in the mITT population (E in FIG. 1; N=44)
12-week Parallel Group
   Placebo: All patients randomized to placebo for the entire duration of the study and included in the mITT population (A/D/G in FIG. 1; N=62)
   AVP-786-18: All patients randomized to AVP-786-18 for the entire duration of the study and included in the mITT population (C/K in FIG. 1; N=94)
   AVP-786-28: All patients randomized to AVP-786-28 for the entire duration of the study and included in the mITT population (B/J in FIG. 1; N=97)

### 5.2. Demographic and Other Baseline Characteristics

Demographic and Baseline characteristics are summarized for the Stage 1 mITT population in Table 15. In general, the groups were balanced with regard to sex (55.8% were female overall), race (91.9% were white, and 6.3% were black), ethnicity (34.8% were Hispanic or Latino), and age (median 76 years overall). A higher proportion of patients in the AVP-786-18 group (16.0%) were <65 years of age than in the placebo (7.3%) or AVP-786-28 (9.3%) groups.

Mean scores on the Baseline efficacy assessments for mITT patients in Stage 1 and Placebo Nonresponders in Stage 2 are presented in Table 16 and Table 17, respectively.

Mean (standard deviation [SD]) CMAI Total scores at Baseline (Stage 1) were similar between treatment groups (Table 16). The mean (SD) CMAI Total score for all patients was 73.0 (22.75). The Baseline means for each of the subscores (F1—Aggressive Behavior, F2—Physically Nonaggressive Behavior, and F3—Verbally Agitated Behavior) were also similar in all 3 groups. Baseline means were also similar across groups for the NPI Total scores, NPI—Agitation/Aggression Domain score, and CGIS-Agitation score.

Mean (SD) CMAI Total scores at the Stage 2 Baseline for Placebo Nonresponders were also similar between treatment groups (66.7 [21.54]; Table 17: Baseline means for the subscores (F1—Aggressive Behavior, F2—Physically Nonaggressive Behavior, and F3—Verbally Agitated Behavior) were also similar in all 3 groups, as were the Baseline mean NPI Total scores, NPI—Agitation/Aggression Domain score, and CGIS-Agitation score.

In the 12-week Parallel Group, Baseline efficacy measures were also similar across groups.

At Baseline, 82.1% of patients were taking at least 1 medication to treat Alzheimer's disease and 43.7% of patient were taking at least 1 medication to treat agitation. There did not appear to be any important differences between treatment groups in the types of medications used at Baseline to treat Alzheimer's disease or agitation.

TABLE 15
Demographics and Baseline Characteristics (Stage 1 mITT Population)

| | Placebo | AVP-786-18 | AVP-786-28 | All Patients |
|---|---|---|---|---|
| Characteristics | (N = 191) | (N = 94) | (N = 97) | (N = 382) |
| Sex n (%) | | | | |
| n | 191 | 94 | 97 | 382 |
| Female | 112 (58.6) | 47 (50.0) | 54 (55.7) | 213 (55.8) |
| Male | 79 (41.4) | 47 (50.0) | 43 (44.3) | 169 (44.2) |
| Race n (%) | | | | |
| n | 191 | 94 | 97 | 382 |
| White | 175 (91.6) | 87 (92.6) | 89 (91.8) | 351 (91.9) |
| Black or African American | 12 (6.3) | 6 (6.4) | 6 (6.2) | 24 (6.3) |
| Asian | 3 (1.6) | 1 (1.1) | 1 (1.0) | 5 (1.3) |
| American Indian or Alaska Native | 0 | 0 | 1 (1.0) | 1 (0.3) |
| Native Hawaiian or Other Pacific Islander | 0 | 0 | 0 | 0 |
| Other | 1 (0.5) | 0 | 0 | 1 (0.3) |
| Ethnicity n (%) | | | | |
| n | 191 | 94 | 97 | 382 |
| Hispanic or Latino | 69 (36.1) | 33 (35.1) | 31 (32.0) | 133 (34.8) |
| Not Hispanic or Latino | 122 (63.9) | 61 (64.9) | 66 (68.0) | 249 (65.2) |
| Age (years) | | | | |
| n | 191 | 94 | 97 | 382 |
| Mean (SD) | 76.6 (7.92) | 73.8 (8.39) | 74.6 (7.69) | 75.4 (8.06) |
| Median | 78.0 | 74.0 | 75.0 | 76.0 |
| Min, Max | 52, 90 | 56, 88 | 51, 89 | 51, 90 |

max = maximum;
min = minimum;
mITT = modified intent-to-treat;
SD = standard deviation
Note:
Denominators are the number of patients who had that parameter assessed.

TABLE 16
Stage 1 Baseline Efficacy Assessments (Stage 1 mITT Population)

| Assessment | Placebo | A VP-786-18 | AVP-786-28 | All Patients |
|---|---|---|---|---|
| Statistics | (N = 191) | (N = 94) | (N = 97) | (N = 382) |
| CMAI-Total score | | | | |
| n | 188 | 92 | 97 | 377 |
| Mean (SD) | 73.9 (22.23) | 72.3 (23.08) | 1.7 (23.57) | 73.0 (22.75) |
| Median | 69.5 | 71.0 | 66.0 | 68.0 |
| Min, Max | 38, 179 | 36, 130 | 37, 125 | 36, 179 |
| CMAI-F1-Aggressive Behavior | | | | |
| n | 188 | 92 | 97 | 377 |
| Mean (SD) | 2.1.0 (8.76) | 21.2 (8.86) | 20.7 (8.70) | 21.0 (8.75) |
| Median | 18.0 | 18.0 | 19.0 | 18.0 |
| Min, Max | 12, 71 | 12, 47 | 12, 52 | 12, 71 |
| CMAI-F2-Physically Nonaggressive Behavior | | | | |
| n | 188 | 92 | 97 | 377 |
| Mean (SD) | 20.7 (8.15) | 20.7 (8.13) | 20.1 (8.73) | 20.5 (8.28) |
| Median | 20.0 | 19.5 | 20.0 | 20.0 |
| Min, Max | 6, 41 | 6, 40 | 6, 41 | 6, 41 |
| CMAI-F3-Verbally Agitated Behavior | | | | |
| n | 188 | 92 | 97 | 377 |
| Mean (SD) | 17.6 (5.91) | 16.7 (6.11) | 17.1 (5.42) | 17.2 (5.83) |
| Median | 18.0 | 17.5 | 17.0 | 17.0 |
| Min, Max | 6, 28 | 4, 28 | 6, 28 | 4, 28 |
| NPI-Total score | | | | |
| n | 191 | 94 | 97 | 382 |
| Mean (SD) | 40.2 (17.95) | 39.4 (18.93) | 40.1 (19.98) | 40.0 (18.68) |
| Median | 39.0 | 38.0 | 38.0 | 39.0 |
| Min, Max | 5, 93 | 2, 111 | 4, 97 | 2, 111 |
| NPI-Agitation/Aggression | | | | |
| n | 191 | 94 | 97 | 382 |
| Mean (SD) | 7.1 (2.39) | 6.5 (1.94) | 7.2 (2.42) | 7.0 (2.30) |
| Median | 6.0 | 6.0 | 6.0 | 6.0 |
| Min, Max | 2, 12 | 1, 12 | 2, 12 | 1, 12 |
| CGIS-Agitation score | | | | |
| n | 191 | 94 | 97 | 382 |
| Mean (SD) | 4.5 (0.64) | 4.3 (0.49) | 4.4 (0.70) | 4.4 (0.62) |
| Median | 4.0 | 4.0 | 4.0 | 4.0 |
| Min, Max | 4, 6 | 4, 6 | 4, 6 | 4, 6 |

max = maximum;
min = minimum;
mITT = modified intent-to-treat:
SD = standard deviation

TABLE 17
Stage 2 Baseline Efficacy Assessments for Placebo Nonresponders
(Stage 2 mITT Population)

| | | Placebo/ | Placebo/ | Placebo |
|---|---|---|---|---|
| Assessment | Placebo | AVP-786-18 | AVP-786-28 | Nonresponders |
| Statistics | (N = 40) | (N = 41) | (N = 44) | (N = 125) |
| CMAI-Total score | | | | |
| n | 40 | 40 | 44 | 124 |
| Mean (SD) | 66.0 (24.71) | 68.9 (20.79) | 65.3 (19.36) | 66.7 (21.54) |
| Median | 62.0 | 61.5 | 58.0 | 60.5 |
| Min, Max | 35, 173 | 41, 123 | 42, 129 | 35, 173 |
| CMAI-F1-Aggressive Behavior | | | | |
| n | 40 | 40 | 44 | 124 |
| Mean (SD) | 19.5 (10.28) | 18.9 (5.92) | 17.9 (6.37) | 18.8 (7.70) |
| Median | 16.0 | 18.0 | 16.0 | 17.0 |
| Min, Max | 12, 70 | 12, 38 | 12, 45 | 12, 70 |
| CMAI-F2-Physically Nonaggressive Behavior | | | | |
| n | 40 | 40 | 44 | 124 |
| Mean (SD) | 18.8 (7.79) | 18.3 (8.94) | 18.6 (8.23) | 18.6 (8.27) |
| Median | 18.0 | 17.0 | 16.0 | 17.0 |
| Min, Max | 6, 39 | 6, 39 | 7, 35 | 6, 39 |
| CMAI-F3-Verbally Agitated Behavior | | | | |
| n | 40 | 40 | 44 | 124 |
| Mean (SD) | 15.2 (5.75) | 17.7 (5.29) | 15.7 (6.23) | 16.2 (5.84) |
| Median | 15.5 | 18.0 | 16.0 | 16.0 |
| Min. Max | 4, 27 | 8, 27 | 4, 27 | 4, 27 |
| NPI-Total score | | | | |
| n | 40 | 41 | 44 | 125 |
| Mean (SD) | 39.2 (22.47) | 34.7 (14.16) | 34.6 (16.93) | 36.1 (18.10) |
| Median | 35.0 | 32.0 | 32.0 | 33.0 |
| Min, Max | 9, 128 | 6, 76 | 1, 73 | 1, 128 |
| NPI-Agitation/Aggression Domain score | | | | |
| n | 40 | 41 | 44 | 125 |
| Mean (SD) | 6.9 (2.84) | 6.3 (2.71) | 6.3 (2.81) | 6.5 (2.78) |
| Median | 6.0 | 6.0 | 6.0 | 6.0 |
| Min, Max | 2, 12 | 1, 12 | 0, 12 | 0, 12 |
| CGIS-Agitation score | | | | |
| n | 40 | 41 | 44 | 125 |
| Mean (SD) | 4.4 (0.87) | 4.2 (0.77) | 4.1 (0.77) | 4.2 (0.81) |
| Median | 4.0 | 4.0 | 4.0 | 4.0 |
| Min, Max | 2, 6 | 2, 6 | 3, 6 | 2, 6 |

max = maximum; min = minimum; mITT = modified intent-to-treat; SD = standard deviation

### 5.3. Efficacy Results and Tabulations of Individual Patient Data

### 5.3.1. Analysis of Efficacy

The following sections present the results of the analyses of the primary (CMAI Total score) and secondary efficacy endpoints. The impact of the gatekeeping procedure on the analyses and interpretation of the primary and key secondary efficacy endpoints are briefly addressed below.

### Statistical Gatekeeping Procedure

As described elsewhere herein, a statistical gatekeeping procedure was used to control the family-wise type 1 error rate (FWE) for both the primary efficacy endpoint (CMAI Total score) and the key secondary efficacy endpoint (mADCS-CGIC-Agitation score). As part of this required gatekeeping procedure, there were 4 fixed sequential treatment comparisons based on efficacy endpoint and AVP-786 dose (AVP-786-18 or AVP-786-28 vs placebo) that were to be performed in the following step-wise manner:
1. CMAI Total score—AVP-786-28 vs placebo
2. mADCS-CGIC-Agitation score—AVP-786-28 vs placebo
3. CMAI Total score—AVP-786-18 vs placebo
4. mADCS-CGIC-Agitation score—AVP-786-18 vs placebo

For example, if the first treatment comparison (CMAI Total score—AVP-786-28 vs placebo) in the sequence did not achieve statistical significance (p<0.05), all the subsequent comparisons in the hierarchy were considered as not significant, regardless of their nominal p-values.

### Results of Gatekeeping Procedure

Based on the results of the gatekeeping procedure, the first comparison in the sequence (CMAI Total score—AVP-786-28 vs placebo) did not achieve statistical significance (p=0.208; Table 21); nor did the second comparison, AVP-786-28 vs placebo for mADCS-CGIC-Agitation score (p=0.097). Although neither dose of AVP-786 showed a significant difference from placebo in CMAI Total score or mADCS-CGIC-Agitation score based on the FWE α=0.05 level, these comparisons were significant for the AVP-786-18 dose at the nominal α=0.05 level (p=0.008 and p=0.012, respectively).

Other secondary efficacy endpoints and subgroup analyses are not impacted by the gatekeeping procedure. Comparisons are performed and reported at the pre-specified nominal 2-sided α=0.05 significance level.

### 5.3.1.1. Primary Efficacy Endpoint

The primary efficacy endpoint is the change from Baseline to Week 6 (Stage 1) and from Week 6 to Week 12 (Stage 2) in the CMAI Total score using the SPCD analysis. The analyses around this primary efficacy endpoint include the following, and are described in the subsequent sections:
SPCD (mITT): combined Stage 1 and Stage 2 (primary analysis), Stage 1, and Stage 2 (Placebo Nonresponders only)
12-week Parallel Group (mITT): change from Baseline to Week 12 (supporting analysis, includes all patients who received the same treatment for the entire duration of the study)
Sensitivity Analyses: SUR, MNAR, OLS/ANCOVA, and SPCD (ITT)

### 5.3.1.1.1. SPCD: Stage 1 and Stage 2 (Placebo Nonresponders)

The primary efficacy endpoint was the SPCD analysis of the change from Baseline in the CMAI Total score for AVP-786-18 and AVP-786-28 versus placebo (Table 22a, Table 21).

In the SPCD analysis, patients treated with AVP-786-18 showed greater improvement in the mean CMAI Total score compared with placebo (significant at the nominal level, p=0.008) (Table 21). For patients treated with AVP-786-28 compared with placebo, the p-value was not significant (p=0.208).

In Stage 1, which mimicked a parallel-group design, patients treated with AVP-786-18 showed greater improvement in the mean CMAI Total score compared with placebo (treatment difference [CI]: −4.0 [−7.4 to −0.6]), which was significant at the nominal level (p=0.021). The treatment difference (CI) for patients treated with AVP-786-28 compared with placebo was −0.6 (−3.9 to 2.7) with a p=0.731.

In Stage 2, which mimicked a parallel-group design with a placebo run-in (Stage 1), patients treated with AVP-786-18 and AVP-786-28 showed greater improvement in the mean CMAI Total score compared with placebo (treatment difference [CI]: (−3.5 [−8.4 to 1.4] and −3.6 [−8.4 to 1.3], respectively), which did not reach significance at the nominal level (p=0.157 and p=0.150, respectively).

TABLE 21
CMAI Total Score: Change from Baseline SPCD MMRM (Observed Data)-mITT Population

| Stage | | | | | | |
|---|---|---|---|---|---|---|
| Parameter/Results | Placebo | | AVP-786-18 | | AVP-786-28 | |
| Stage 1 | | | | | | |
| Baseline N, Mean (SD) | 188, 73.9 | (22.23) | 92, 72.3 | (23.08) | 97, 71.7 | (23.57) |
| Week 6 Change from Baseline: LS Mean (SB) [1] | −10.3 | (1.21) | −14.3 | (1.61) | −10.9 | (1.55) |
| Treat Diff vs. Placebo: p-value (Dif, 95% CI) [1] | | | 0.021 | (−4.0, −7.4 to −0.6) | 0.731 | (−0.6, −3.9 to 2.7) |
| Stage 2 (Stage 1 Placebo Nonresponders only) | | | | | | |
| Stage 2 Baseline: N, Mean (SD) [2] | 40, 66.0 | (24.71) | 40, 68.9 | (20.79) | 44, 65.3 | (19.36) |
| Week 12 Change from Baseline: LS Mean (SE) [1] | −1.7 | (1.76) | −5.2 | (1.75) | −5.2 | (1.71) |
| Treat Diff vs. Placebo: p-value (Dif, 95% CI) [1] | | | 0.157 | (−3.5, −8.4 to 1.4) | 0.150 | (−3.6, −8.4 to 1.3) |
| SPCD Week 6 & 12 MMRM weighted z-statistic, | | | −2.65, p = 0.008 | | −1.26, p = 0.208 | |
| p-value [3] | | | | | | |

Note:
CMAI Total score ranges from 29 to 203 with higher scores indicating worsening condition.
[1] MMRMs include fixed effects of treatment, visit, treatment-by-visit. Baseline, Baseline-by-visit, and in the Stage 1 model, Baseline NPI AA (≤6 vs >6), risk assessment for falls (normal/mild vs moderate/severe). Baseline concomitant use of antipsychotic medications (yes vs no). Unstructured variance-covariance was used. Stage 1 and 2 Baseline values were used in their respective models.
[2] Stage 2 Baseline is the last nonmissing assessment prior to Stage 2 rerandomization (rerandomization visit).
[3] SPCD Week 6 & 12 MMRM weighted z-statistic was used with weight = 0.6 for Slage 1 and 0.4 for Stage 2.

### 5.3.1.1.2. 12-Week Parallel Group

The change from Baseline in the mean CMAI Total score is presented for the 12-week Parallel Group in Table 22 and in Table 22b. Patients treated with AVP-786-18 showed greater improvement in the mean CMAI Total score compared with placebo (treatment difference [CI]: −4.9 [−9.6 to −0.2]), which was significant at the nominal level (p=0.042). The treatment difference (CI) for patients treated with AVP-786-28 compared with placebo was −1.4 (−6.0 to 3.2) with a p=0.555.

TABLE 22
CMAL: Change from Baseline Parallel Group MMRM Analysis-Observed Data
(mITT 12-week Parallel Group Population)

| Parameter/Results | Placebo [1] | | AVP-786-18 [1] | | AVP-786-28 [1] | |
|---|---|---|---|---|---|---|
| Baseline: N, Mean(SD) | 62, 70.9 | (21.98) | 92, 72.3 | (23.08) | 97, 71.7 | (23.57) |
| Week 12 Change from Baseline: LS Mean (SE) [2] | −10.7 | (2.02) | −15.6 | (1.77) | −12.1 | (1.72) |
| Treat Diff vs. Placebo: p-value (Dif, 95% CI) [2] | | | 0.042 | (−4.9, −9.6 to −0.2) | 0.555 | (−1.4, −6.0 to 3.2) |

Note:
CMAI Total score ranges from 29 to 203 with higher scores indicating worsening condition.
[1] Patients who are randomized to the same treatment group in both stages.
[2] MMRM with fixed effects of treatment, visit, treatment-by-visit. Baseline, Baseline-by-visit, Baseline NPI AA (≤6 vs. >6), risk assessment for falls (notmal/mild vs moderate/sevete). Baseline concomitant use of antipsychotic medications (yes vs no). Unstructured variance-covariance was used.

The change from Baseline in the mean CMAI Total score at various time points is shown in the Tables 22a and 22b below:

TABLE 22a
CMAI: Change from Baseline SPCD MMRM-Observed Data (mITT Population)
CMAI: Total Score

| Stage | Parameter/Results | Placebo | | AVP-786 18 mg | | AVP-786 28 mg | |
|---|---|---|---|---|---|---|---|
| Stage 1 | Baseline: N, Mean (SD) | 188, 73.9 | (22.23) | 92, 72.3 | (23.08) | 97, 71.7 | (23.57) |
| | Week 1 Change from Baseline: N, Mean (SD) | 182, −5.8 | (9.78) | 88, −6.3 | (12.60) | 94, −6.4 | (9.62) |
| | Change from Baseline: LS Mean (SE) [1] | −5.6 | (1.03) | −6.5 | (1.30) | −6.5 | (1.27) |
| | Treat Diff vs. Placebo: p-value (Dif, 95% CI) [1] | 141, −7.4 | (11.55) | 0.499 | (−0.9, −3.4 to 1.7) | 0.458 | (−0.9, −3.4 to 1.5) |
| | Week 2 Change from Baseline: N, Mean (SD) | −7.0 | (1.09) | 70, −9.2 | (14.69) | 70, −6.8 | (10.56) |
| | Change from Baseline: LS Mean (SE) [1] | 184, −8.9 | (13.84) | −9.1 | (1.40) | −6.7 | (1.38) |
| | Treat Diff vs. Placebo: p-value (Dif, 95% CI) [1] | −8.7 | (1.17) | 0.139 | (−2.1, −5.0 to 0.7) | 0.838 | (0.3, −2.5 to 3.1) |
| | Week 3 Change from Baseline: N, Mean (SD) | 180, −10.7 | (15.29) | 87, −11.8 | (16.09) | 95, −9.0 | (12.90) |
| | Change from Baseline: LS Mean (SE) [1] | −10.3 | (1.21) | −11.9 | (1.54) | −9.4 | (1.50) |
| | Treat Diff vs. Placebo: p-value (Dif, 95% CI) [1] | | | 0.047 | (−3.2, −6.5 to −0.0) | 0.633 | (−0.8, −3.9 to 2.4) |
| | Week 6 Change from Baseline: N, Mean (SD) | | | 85, −13.9 | (16.70) | 94, −10.3 | (13.54) |
| | Change from Baseline: LS Mean (SE) [1] | | | −14.3 | (1.61) | −10.9 | (1.55) |
| | Treat Diff vs. Placebo: p-value (Dif, 95% CI) [1] | | | 0.021 | (−4.0, −7.4 to −0.6) | 0.731 | (−0.6, −3.9 to 2.7) |
| Stage 2 | Stage 2 Baseline: N, Mean (SD) [2] | 40, 66.0 | (24.71) | 40, 68.9 | (20.79) | 44, 65.3 | (19.36) |
| (Stage 1 | Week 9 Change from Baseline: N, Mean (SD) | 40, −1.0 | (9.40) | 40, −5.7 | (9.40) | 44, −2.8 | (10.13) |
| Placebo | Change from Baseline: LS Mean (SE) [1] | −1.1 | (1.42) | −5.3 | (1.43) | −3.0 | (1.36) |
| Non- | Treat Diff vs. Placebo: p-value (Dif, 95% CI) [1] | 39, −1.6 | (11.08) | 0.039 | (−4.2, −8.2 to −0.2) | 0.322 | (−2.0, −5.8 to 1.9) |
| responders | Week 12 Change from Baseline: N, Mean (SD) | −1.7 | (1.76) | 40, −5.6 | (11.78) | 41, −4.9 | (12.13) |
| only) | Change from Baseline: LS Mean (SE) [1 ] | | | −5.2 | (1.75) | -5.2 | (1.71) |
| | Treat Diff vs. Placebo: p-value (Dif, 95% CI) [1] | | | 0.157 | (−3.5, −8.4 to 1.4) | 0.150 | (−3.6, −8.4 to 1.3) |
| | SPCD Week 6 & 12 MMRM weighted | | | −2.65, p = 0.008 | | −1.26, p = 0.208 | |
| | z-statistic, p-value [3] | | | | | | |

Note:
CMAI Total Score ranges from 29 to 203 with higher scores indicating worsening condition.
[1] MMRMs include fixed effects of treatment, visit, treatment-by-visit, baseline, baseline-by-visit, and in the Stage 1 model, baseline NPI AA (≤6 vs. >6), risk assessment for fails (normal/mild vs. moderate/severe), baseline concomitant use of antipsychotic medications (yes vs. no). Unstructured variance-covariance was used. Stage 1 and 2 baseline values were used in their respective models.
[2] Stage 2 Baseline is the last non-missing assessment prior to Stage 2 re-randomization (re-randomization visit).
[3] SPCD Week 6 & 12 MMRM weighted z-statistic was used with weight = 0.6 for Stage 1 and 0.4 for Stage 2.

TABLE 22b
CMAL: Change from Baseline Parallel Group MMRM Analysis-Observed Data (mITT 12-Week Parallel Group Population)
CMAI: Total Score

| Parameter/Results | Placebo [1] | | AVP-786 18 mg [1] | | AVP-786 28 mg [1] | |
|---|---|---|---|---|---|---|
| Baseline: N, Mean (SD) | 62, 70.9 | (21.98) | 92, 72.3 | (23.08) | 97, 71.7 | (23.57) |
| Week 1 Change from Baseline: N, Mean (SD) | 60, −6.1 | (9.57) | 88, −6.3 | (12.60) | 94, −6.4 | (9.62) |
| Change from Baseline: LS Mean (SE) [1] | −6.0 | (1.56) | −6.3 | (1.42) | −6.4 | (1.39) |
| Treat Diff vs. Placebo: p-value (Dif, 95% CI) [2] | | | 0.852 | (−0.3, −3.7 to 3.1) | 0.828 | (−0.4, −3.7 to 3.0) |
| Week 2 Change from Baseline: N, Mean (SD) | 48, −6.4 | (11.89) | 70, −9.2 | (14.69) | 70, −6.8 | (10.56) |
| Change from Baseline: LS Mean (SE) [1] | −6.5 | (1.69) | −8.8 | (1.51) | −6.5 | (1.49) |
| Treat Diff vs. Placebo: p-value (Dif, 95% CI) [2] | | | 0.222 | (−2.3. −6.1 to 1.4) | 0.988 | (0.0, −3.7 to 3.7) |
| Week 3 Change from Baseline: N, Mean (SD) | 61, −7.5 | (15.44) | 87, −11.8 | (16.09) | 95, −9.0 | (12.90) |
| Change from Baseline: LS Mean (SE) [1] | −7.6 | (1.87) | −11.5 | (1.66) | −9.0 | (1.61) |
| Treat Diff vs. Placebo: p-value (Dif, 95% CI) [2] | | | 0.072 | (−3.9, −8.2 to 0.3) | 0.503 | (−1.4, −5.6 to 2.8) |
| Weeks Change from Baseline: N, Mean (SD) | 60, −9.0 | (15.95) | 85, -13.9 | (16.70) | 94, −10.3 | (13.54) |
| Change from Baseline: LS Mean (SE) [1] | −9.1 | (1.93) | −13.8 | (1.71) | −10.5 | (1.65) |
| Treat Diff vs. Placebo: p-value (Dif, 95% CI) [2] | | | 0.038 | (−4.7, −9.2 to −0.3) | 0.520 | (−1.4, −5.8 to 2.9) |
| Week 9 Change from Baseline: N, Mean (SD) | 57, −10.0 | (15.88) | 84, −14.6 | (18.07) | 91, −11.0 | (14.96) |
| Change from Baseline: LS Mean (SE) [1] | −10.3 | (2.04) | −14.8 | (1.79) | −11.2 | (1.73) |
| Treat Diff vs. Placebo: p-value (Dif, 95% CI) [2] | | | 0.064 | (−4.5, −9.2 to 0.3) | 0.711 | (−0.9, −5.6 to 3.8) |
| Week 12 Change from Baseline: N, Mean (SD) | 57, −10.8 | (16.75) | 85, −15.4 | (17.72) | 92, −12.0 | (13.97) |
| Change from Baseline: LS Mean (SE) [1] | −10.7 | (2.02) | −15.6 | (1.77) | −12.1 | (1.72) |
| Treat Diff vs. Placebo: p-value (Dif, 95% CI) [2] | | | 0.042 | (−4.9, −9.6 to −0.2) | 0.555 | (−1.4, −6.0 to 3.2) |

Note:
CMAI Total Score ranges from 29 to 203 with higher scores indicating worsening condition.
[1] Patients who are randomized to the same treatment group in both stages.
[2] MMRM with fixed effects of treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no). Unstructured variance-covariance was used.

### 5.3.1.1.3. Sensitivity Analyses

Sensitivity analyses on the primary efficacy endpoint using different statistical analyses methods (SUR method and SPCD OLS ANCOVA [LOCF and WOCF+LOCF] and SPCD MMRM with MNAR inference) corroborated the findings of the primary analysis; significant differences between treatment groups AVP-786-18 and placebo, in favor of AVP-786-18 were observed with the SUR method (p=0.006), SPCD OLS ANCOVA-LOCF (p=0.007), SPCD OLS ANCOVA-WOCF+LOCF (p=0.007), and MMRM SPCD using ITT population (p=0.008). A summary of the results is provided in Table 23.

TABLE 23
Summary of Primary and Sensitivity Analysis of the CMAI Total Score

| | Stage 1 | | | | | | Stage 2 | | | | | |
|---|---|---|---|---|---|---|---|---|---|---|---|---|
| | Placebo | | AVP-786-18 | | AVP-786-28 | | Placebo | | AVP-786-18 | | AVP-786-28 | |
| Primary Analysis | | | | | | | | | | | | |
| N, Mean (SD) [1] | 180, −10.7 | (15.29) | 85, −13.9 | (16.70) | 94, −10.3 | (13.54) | 39, −1.6 | (11.08) | 40, −5.6 | (11.78) | 41, −4.9 | (12.13) |
| Dif, 95% CI [2] | | | 4.0 (−7.4 to −0.6) | | −0.6 (−3.9 to 2.7) | | | | −3.5 (−8.4 to 1.4) | | −3.6 (−8.4 to 1.3) | |
| LS mean change from | −10.3 | (1.21) | −14.3 | (1.61) | −10.9 | (1.55) | −1.7 | (1.76) | −5.2 | (1.75) | −5.2 | (1.71) |
| Baseline (SE) [2] | | | | | | | | | | | | |
| p-value | | | 0.021 | | 0.731 | | | | 0.157 | | 0.150 | |
| SPCD p-value [3] | | | | | | | | | 0.008 | | 0.208 | |
| Secondary Analyses | | | | | | | | | | | | |
| SPCD ANCOVA - | | | | | | | | | | | | |
| LOCF | | | | | | | | | | | | |
| N, Mean (SD) [1] | 188, −10.2 | (15.20) | 92, −13.7 | (16.42) | 97, −10.4 | (13.49) | 40, −1.5 | (10.97) | 40, −5.6 | (11.78) | 44, −4.9 | (11.71) |
| Dif, 95% CI [4] | | | −4.0 (−7.3 to −0.7) | | −0.9 (−4.1 to 2.4) | | | | −3.6 (−8.4 to 1.3) | | −3.5 (−8.2 to 1.3) | |
| LS mean change from | −9.5 | (1.38) | −13.5 | (1.73) | −10.4 | (1.70) | −1.6 | (1.73) | −5.2 | (1.73) | −5.1 | (1.65) |
| Baseline (SE) [2] | | | | | | | | | | | | |
| p-value | | | 0.019 | | 0.607 | | | | 0.146 | | 0.151 | |
| SPCD p-value [3] | | | | | | | | | 0.007 | | 0.169 | |
| SPCD ANCOVA - | | | | | | | | | | | | |
| WOCF + LOCF | | | | | | | | | | | | |
| N, Mean (SD) [1] | 188, −10.2 | (15.20) | 92, −13.7 | (16.42) | 97, −10.4 | (13.49) | 40, −1.5 | (10.97) | 40, −5.6 | (11.78) | 44, −4.9 | (11.71) |
| Dif, 95% CI [5] | | | −4.0 (−7.3 to −0.7) | | −0.9 (−4.1 to 2.4) | | | | −3.6 (−8.4 to 1.3) | | −3.5 (−8.2 to 1.3) | |
| LS mean change from | −9.5 | (1.38) | −13.5 | (1.73) | −10.4 | (1.70) | −1.6 | (1.73) | −5.2 | (1.73) | −5.1 | (1.65) |
| Baseline (SE) [2] | | | | | | | | | | | | |
| p-value | | | 0.019 | | 0.607 | | | | 0.146 | | 0.151 | |
| SPCD p-value [3] | | | | | | | | | 0.007 | | 0.169 | |
| SPCD SUR - LOCF | | | | | | | | | | | | |
| N, Mean (SD) [1] | 188, −10.2 | (15.20) | 92, −13.7 | (16.42) | 97, −10.4 | (13.49) | 40, −1.5 | (10.97) | 40, −5.6 | (11.78) | 44, −4.9 | (11.71) |
| Dif, 95% CI | | | −4.1 (−7.5 to −0.7) | | −0.8 (−4.0 to 2.4) | | | | −3.5 (−8.2 to 1.2) | | −3.4 (−8.0 to 1.2) | |
| p-value | | | 0.019 | | 0.620 | | | | 0.146 | | 0.148 | |
| SPCD p-value [3] | | | | | | | | | 0.006 | | 0.172 | |
| SPCD MMRM - | | | | | | | | | | | | |
| Observed Data (ITT) | | | | | | | | | | | | |
| N, Mean (SD) [1] | 180, −10.7 | (15.29) | 85, −13.9 | (16.70) | 94, −10.3 | (13.54) | 39, −1.6 | (11.08) | 40, −5.6 | (11.78) | 41, −4.9 | (12.13) |
| Dif, 95% CI | | | −4.0 (−7.4 to −0.6) | | −0.6 (−3.9 to 2.7) | | | | −3.5 (−8.4 to 1.4) | | −3.6 (−8.4 to 1.3) | |
| LS mean change from | −10.3 | (1.21) | −14.3 | (1.61) | −10.9 | (1.55) | −1.7 | (1.76) | −5.2 | (1.75) | −5.2 | (1.71) |
| Baseline (SE) [2] | | | | | | | | | | | | |

| p-value | 0.021 | 0.731 | 0.157 | 0.150 |
|---|---|---|---|---|
| SPCD p-value [6] | | | 0.008 | 0.208 |

[1] Stage 2 Baseline is the last nonmissing assessment prior to Stage 2 rerandomization (rerandomization visit).
[2] MMRMs include fixed effects of treatment, visit, treatment-by-visit, Baseline, Baseline-by-visit, and in the Stage 1 model, Baseline NPI AA (≤6 vs >6), risk assessment for falls (normal/mild vs moderate/severe), Baseline concomitant use of antipsychotic medications (yes vs no). Unstructured variance-covariance was used. Stage 1 and 2 Baseline values were used in their respective models.
[3] Weighted z-statistic for each analysis was used with weight = 0.6 for Stage 1 and 0.4 for Stage 2.
[4] Treatment effects were estimated at each stage by ANCOVA with fixed effects for treatment, Baseline, and in the Stage 1 model, Baseline NPI AA (≤6 vs >6), risk assessments for falls (normal/mild vs moderate/severe), Baseline concomitant use of antipsychotic medications (yes vs no). Stage 1 and 2 models use their respective Baseline values. Missing values were imputed by LOCF within each stage.
[5] Treatment effects were estimated at each stage by ANCOVA with fixed effects for treatment, Baseline, and in the Stage 1 model, Baseline NPI AA (≤6 vs >6), risk assessments for falls (normal/mild vs moderate/severe), Baseline concomitant use of antipsychotic medications (yes vs no). Stage 1 and 2 models use their respective Baseline values. Missing values were imputed by WOCF + LOCF within each stage.
[6] MMRMs include fixed effects of treatment, visit, treatment-by-visit, Baseline, Baseline-by-visit, and in the Stage 1 model, Baseline NPI AA (≤6 vs >6), risk assessment for falls (normal/mild vs moderate/severe), Baseline concomitant use of antipsychotic medications (yes vs no). Unstructured variance-covariance was used. Stage 1 and 2 Baseline values were used in their respective models.

### 5.3.1.2. CMAI Subscales

The CMAI subscales F1—Aggressive Behavior, F2—Physically Nonaggressive Behavior, and F3—Verbally Agitated Behavior are summarized in Table 24 for the SPCD analysis and in Table 25 for the 12-week Parallel Group, and the subscales are discussed individually below.

TABLE 24
CMAI Subscale Scores: Change from Baseline SPCD MMRM
(Observed Data)-mITT Population

| Parameter/Results | Placebo | | AVP-786-18 | | AVP-786-28 | |
|---|---|---|---|---|---|---|
| CMA: F1-Aggressive Behavior | | | | | | |
| Stage 1 | | | | | | |
| Baseline: N, Mean (SD) | 188, 21.0 | (8.76) | 92, 21.2 | (8.86) | 97, 20.7 | (8.70) |
| Week 6 Change from Baseline: LS Mean (SE) [1] | −3.2 | (0.45) | −4.4 | (0.59) | −3.4 | (0.57) |
| Treat Diff vs. Placebo: p-value (Dif. 95% CI) [1] | | | 0.047 | (−1.2, −2.4 to −0.0) | 0.731 | (−0.2, −1.4 to 1.0) |
| Stage 2 (Stage 1 Nonresponders only) | | | | | | |
| Stage 2 Baseline: N, Mean (SD) [2] | 40, 19.5 | (10.28) | 40, 18.9 | (5.92) | 44, 17.9 | (6.37) |
| Week 12 Change from Baseline: LS Mean (SE) [1] | −0.2 | (0.78) | −1.8 | (0.77) | −1.4 | (0.75) |
| Treat Diff vs. Placebo: p-value (Dif, 95% CI) [1] | | | 0.158 | (−1.5, −3.7 to 0.6) | 0.280 | (−1.2, −3.3 to 1.0) |
| SPCD Week 6 & 12 MMRM weighted z-statistic, | | | −2.37, p = 0.018 | | −1.05, p = 0.292 | |
| p-value (3} | | | | | | |
| CMAI: F2-Physically Nonaggressive Behavior | | | | | | |
| Stage 1 | | | | | | |
| Baseline: N, Mean (SD) | 188, 20.7 | (8.15) | 92, 20.7 | (8.13) | 97, 20.1 | (8.73) |
| Week 6 Change from Baseline: LS Mean (SE) [1] | −3.1 | (0.44) | −4.8 | (0.59) | −3.5 | (0.57) |
| Treat Diff vs. Placebo: p-value (Dif, 95% CI) [1] | | | 0.011 | (−1.6, −2.9 to −0.4) | 0.511 | (−0.4, −1.6 to 0.8) |
| Stage 2 (Stage 1 Nonresponders only) | | | | | | |
| Stage 2 Baseline: N, Mean (SD) [2] | 40, 18.8 | (7.79) | 40, 18.3 | (8.94) | 44, 18.6 | (8.23) |
| Week 12 Change from Baseline: LS Mean (SE) [1] | −0.1 | (0.61) | −0.7 | (0.60) | −1.9 | (0.59) |
| Treat Diff vs. Placebo: p-value (Dif, 95% CI) [1] | | | 0.475 | (−0.6, −2.3 to 1.1) | 0.043 | (−1.7, −3.4 to −0.1) |
| SPCD Week 6 & 12 MMRM weighted z-statistic, | | | −2.38, p = 0.017 | | −1.87, p = 0.062 | |
| p-value [3] | | | | | | |
| CMAI: F3-Verbally Agitated Behavior | | | | | | |
| Stage 1 | | | | | | |
| Baseline: N, Mean (SD) | 188, 17.6 | (5.91) | 92, 16.7 | (6.11) | 97, 17.1 | (5.42) |
| Week 6 Change from Baseline: LS Mean (SE) [1] | −2.2 | (0.38) | −3.0 | (0.50) | −2.5 | (0.48) |
| Treat Diff vs. Placebo: p-value (Dif. 95% CI) [1] | | | 0.145 | (−0.8, −1.8 to 0.3) | 0.571 | (−0.3, −1.3 to 0.7) |
| Stage 2 (Stage 1 Placebo Nonresponders only) | | | | | | |
| Stage 2 Baseline: N, Mean (SD) [2] | 40, 15.2 | (5.75) | 40, 17.7 | (5.29) | 44, 15.7 | (6.23) |
| Week 12 Change from Baseline: LS Mean (SE) [1] | −0.9 | (0.59) | −2.1 | (0.59) | −1.3 | (0.58) |
| Treat Diff vs. Placebo: p-value (Dif. 95% CI) [1] | | | 0.165 | (−1.2, −2.9 to 0.5) | 0.657 | (−0.4, −2.0 to 1.3) |
| SPCD Week 6 & 12 MMRM weighted z-statistic, | | | −2.02, p = 0.044 | | −0.71, p = 0.476 | |
| p-value [3] | | | | | | |

Note:
Factor 1, Aggressive Behavior ranges from 12 to 84. Factor 2, F2-Physically Nonaggressive Behavior ranges 6 to 42. Factor 3, F3-Verbally Agitated Behavior ranges 4 to 28. For all factors, higher scores indicating worsening condition.
[1] MMRMs include fixed effects of treatment, visit, treatment-by-visit. Baseline, Baseline-by-visit, and in the Stage 1 model, Baseline NPI AA (≤6 vs. >6), risk assessment for falls (normal/mild vs. moderate/severe). Baseline concomitant use of antipsychotic medications (yes vs. no). Unstructured variance-covariance was used. Stage 1 and 2 Baseline values were used in their respective models.
[2] Stage 2 Baseline is the last: nonmissing assessment prior to Stage 2 terandomization (rerandomization visit).
[3] SPCD Week 6 & 12 MMRM weighted z-statistic was used with weight = 0.6 for Stage 1 and 0.4 for Stage 2.

The changes from Baseline in the mean CMAI Aggressive Behavior scores, CMAI Nonaggressive Behavior scores, and CMAI Verbal Agitation scores at various time points are shown in the Tables 24a-24f below:

TABLE 24a
CMAI: Change from Baseline SPCD MMRM-Observed Data (mITT Population)
CMAI: Aggressive Behavior

| Stage | Parameter/Results | Placebo | | AVP-786 18 mg | | AVP-786 28 mg | |
|---|---|---|---|---|---|---|---|
| Stage 1 | Baseline: N, Mean (SD) | 188, 21.0 | (8.76) | 92, 21.2 | (8.86) | 97, 20.7 | (8.70) |
| | Week 1 Change from Baseline: N, Mean (SD) | 181, −1.9 | (4.28) | 88, −2.0 | (4.86) | 94, −1.7 | (4.48) |
| | Change from Baseline: LS Mean (SE) [1] | −1.8 | (0.40) | −1.9 | (0.51) | −1.7 | (0.50) |
| | Treat Diff vs. Placebo: p-value (Dif, 95% CI) [1] | 141, −2.6 | (5.16) | 0.772 | (−0.1, −1.2 to 0.9) | 0.844 | (0.1, −0.9 to 1.1) |
| | Week 2 Change from Baseline: N, Mean (SD) | −2.3 | (0.43) | 70, −3.0 | (6.84) | 70, −2.3 | (4.60) |
| | Change from Baseline: LS Mean (SE) [1] | 184, −2.8 | (5.72) | −2.8 | (0.56) | −2.1 | (0.55) |
| | Treat Diff vs. Placebo: p-value (Dif, 95% CI) [1] | −2.6 | (0.43) | 0.333 | (−0.6, −1.7 to 0.6) | 0.723 | (0.2, −0.9 to 1.3) |
| | Week 3 Change from Baseline: N, Mean (SD) | 180, −3.3 | (6.56) | 87, −4.0 | (6.87) | 95, −2.6 | (5.04) |
| | Change from Baseline: LS Mean (SE) [1] | −3.2 | (0.45) | −3.8 | (0.56) | −2.7 | (0.55) |
| | Treat Diff vs. Placebo: p-value (Dif, 95% CI) [1] | | | 0.036 | (−1.2, −2.4 to −0.1) | 0.914 | (−0.1, −1.2 to 1.1) |
| | Week 6 Change from Baseline: N, Mean (SD) | | | 85, −4.3 | (7.05) | 94, −3.3 | (5.01) |
| | Change from Baseline: LS Mean (SE) [1] | | | −4.4 | (0.59) | −3.4 | (0.57) |
| | Treat Diff vs. Placebo: p-value (Dif, 95% CI) [1] | | | 0.047 | (−1.2, −2.4 to −0.0) | 0.731 | (−0.2, −1.4 to 1.0) |
| Stage 2 | Stage 2 Baseline: N, Mean (SD) [2] | 40, 19.5 | (10.28) | 40, 18.9 | (5.92) | 44, 17.9 | (6.37) |
| (Stage 1 | Week 9 Change from Baseline: N, Mean (SD) | 40, −0.3 | (4.44) | 40, −2.2 | (3.40) | 44, −0.2 | (3.83) |
| Placebo | Change from Baseline: LS Mean (SE) [1] | −0.1 | (0.57) | −2.2 | (0.57) | −0.4 | (0.54) |
| Non- | Treat Diff vs. Placebo: p-value (Dif, 95% CI) [1] | 39, −0.5 | (5.46) | 0.013 | (−2.0, −3.6 to −0.4) | 0.783 | (−0.2, −1.8 to 1.3) |
| responders | Week 12 Change from Baseline: N, Mean (SD) | −0.2 | (0.78) | 40, −1.8 | (5.23) | 41, −1.3 | (4.81) |
| only) | Change from Baseline: LS Mean (SE) [1] | | | −1.8 | (0.77) | −1.4 | (0.75) |
| | Treat Diff vs. Placebo: p-value (Dif, 95% CI) [1] | | | 0.158 | (−1.5, −3.7 to 0.6) | 0.280 | (−1.2, −3.3 to 1.0) |
| | SPCD Week 6 & 12 MMRM weighted | | | −2.37, p = 0.018 | | −1.05, p = 0.292 | |
| | z-statistic, p-value [3] | | | | | | |

Note:
Factor 1, Aggressive Behavior ranges from 12 to 84 with higher scores indicating worsening condition.
[1] MMRMs include fixed effects of treatment, visit, treatment-by-visit, baseline, baseline-by-visit, and in the Stage 1 model, baseline NPI AA (≤6 vs. >6), risk assessment for falls (normal/mild vs. moderate/severe), baseline concomitant use of antipsychotic medications (yes vs. no). Unstructured variance-covariance was used. Stage 1 and 2 baseline values were used in their respective models.
[2] Stage 2 Baseline is the last non-missing assessment prior to Stage 2 re-randomization (re-randomization visit).
[3] SPCD Week 6 & 12 MMRM weighted z-statistic was used with weight = 0.6 for Stage 1 and 0.4 for Stage 2.

TABLE 24b
CMAI: Change from Baseline Parallel Group MMRM Analysis-Observed Data (mITT 12-Week Parallel Group Population)
CMAI: Aggressive Behavior

| Parameter/Results | Placebo [1] | | AVP-786 18 mg [1] | | AVP-786 28 mg [1] | |
|---|---|---|---|---|---|---|
| Baseline: N, Mean (SD) | 62, 21.0 | (10.14) | 92, 21.2 | (8.86) | 97, 20.7 | (8.70) |
| Week 1 Change from Baseline: N, Mean (SD) | 60, −2.3 | (3.88) | 88, −2.0 | (4.86) | 94, −1.7 | (4.48) |
| Change from Baseline: LS Mean (SE) [1] | −2.1 | (0.62) | −2.0 | (0.57) | −1.7 | (0.55) |
| Treat Diff vs. Placebo: p-value (Dif, 95% CI) [2] | | | 0.824 | (0.2, −1.2 to 1.5) | 0.549 | (0.4, −0.9 to 1.7) |
| Week 2 Change from Baseline: N, Mean (SD) | 48, −2.0 | (5.04) | 70, −3.0 | (6.84) | 70, −2.3 | (4.60) |
| Change from Baseline: LS Mean (SE) [1] | −1.9 | (0.69) | −2.9 | (0.62) | −2.1 | (0.61) |
| Treat Diff vs. Placebo: p-value (Dif, 95% CI) [2] | | | 0.190 | (−1.0, −2.6 to 0.5) | 0.752 | (−0.2, −1.8 to 1.3) |
| Week 3 Change from Baseline: N, Mean (SD) | 61, −2.6 | (6.14) | 87, −4.0 | (6.87) | 95, −2.6 | (5.04) |
| Change from Baseline: LS Mean (SE) [1] | −2.4 | (0.70) | −3.9 | (0.63) | −2.7 | (0.61) |
| Treat Diff vs. Placebo: p-value (Dif, 95% CI) [2] | | | 0.058 | (−1.5, −3.1 to 0.0) | 0.663 | (−0.3, −1.9 to 1.2) |
| Week 6 Change from Baseline: N, Mean (SD) | 60, −2.9 | (7.23) | 85, −4.3 | (7.05) | 94, −3.3 | (5.01) |
| Change from Baseline: LS Mean (SE) [1] | −2.7 | (0.72) | −4.4 | (0.64) | −3.4 | (0.62) |
| Treat Diff vs. Placebo: p-value (Dif, 95% CI) [2] | | | 0.040 | (−1.7, −3.3 to −0.1) | 0.406 | (−0.7, −2.3 to 0.9) |
| Week 9 Change from Baseline: N, Mean (SD) | 57, −3.2 | (6.35) | 84, −4.5 | (7.63) | 91, −3.2 | (5.60) |
| Change from Baseline: LS Mean (SE) [1] | −3.3 | (0.74) | −4.7 | (0.66) | −3.5 | (0.64) |
| Treat Diff vs. Placebo: p-value (Dif, 95% CI) [2] | | | 0.098 | (−1.4, −3.1 to 0.3) | 0.787 | (−0.2, −1.9 to 1.4) |
| Week 12 Change from Baseline: N, Mean (SD) | 57, −3.5 | (7.58) | 85, −4.6 | (7.25) | 92, −3.5 | (6.05) |
| Change from Baseline: LS Mean (SE) [1] | −3.3 | (0.79) | −4.8 | (0.70) | −3.8 | (0.68) |
| Treat Diff vs. Placebo: p-value (Dif, 95% CI) [2] | | | 0.117 | (−1.5, −3.3 to 0.4) | 0.646 | (−0.4, −2.2 to 1.4) |

Note:
Factor 1, Aggressive Behavior ranges from 12 to 84 with higher scores indicating worsening condition.
[1] Patients who are randomized to the same treatment group in both stages.
[2] MMRM with fixed effects of treatment, visit, treatment-by-visit, baseline, baseline-by-visit, baseline NPI AA (≤6 vs. >6), risk assessment for falls (normai/mild vs. moderate/severe), baseline concomitant use of antipsychotic medications (yes vs. no). Unstructured variance-covariance was used.

TABLE 24c
CMAI: Change from Baseline SPCD MMRM-Observed Data (mITT Population)
CMAI: Physically Non-Aggressive Behavior

| Stage | Parameter/Results | Placebo | | AVP-786 18 mg | | AVP-786 28 mg | |
|---|---|---|---|---|---|---|---|
| Stage 1 | Baseline: N, Mean (SD) | 188, 20.7 | (8.15) | 92, 20.7 | (8.13) | 97, 20.1 | (8.73) |
| | Week 1 Change from Baseline: N, Mean (SD) | 182, −1.7 | (3.74) | 88, −2.2 | (4.21) | 94, −1.9 | (3.30) |
| | Change from Baseline: LS Mean (SE) [1] | −1.8 | (0.37) | −2.3 | (0.47) | −2.0 | (0.46) |
| | Treat Diff vs. Placebo: p-value (Dif, 95% CI) [1] | 141, −2.0 | (4.18) | 0.278 | (−0.5, −1.4 to 0.4) | 0.600 | (−0.2, −1.1 to 0.7) |
| | Week 2 Change from Baseline: N, Mean (SD) | −2.1 | (0.39) | 70, −2.8 | (4.26) | 70, −1.9 | (3.54) |
| | Change from Baseline: LS Mean (SE) [1] | 184, −2.2 | (5.13) | −3.0 | (0.50) | −2.0 | (0.49) |
| | Treat Diff vs. Placebo: p-value (Dif, 95% CI) [1] | −2.4 | (0.43) | 0.091 | (−0 9, −1.9 to 0.1) | 0.848 | (0.1, −0.9 to 1.1) |
| | Week 3 Change from Baseline: N, Mean (SD) | 180, −3.0 | (5.41) | 87, −3.8 | (5.14) | 95, −2.9 | (5.00) |
| | Change from Baseline: LS Mean (SE) [1] | −3.1 | (0.44) | −3.8 | (0.58) | −3.2 | (0.56) |
| | Treat Diff vs. Placebo: p-value (Dif, 95% CI) [1] | | | 0.022 | (−1.4, −2.6 to −0.2) | 0.174 | (−0.8, −2.0 to 0.4) |
| | Week 6 Change from Baseline: N, Mean (SD) | | | 85, −4.7 | (5.29) | 94, −3.2 | (5.04) |
| | Change from Baseline: LS Mean (SE) [1] | | | −4.8 | (0.59) | −3.5 | (0.57) |
| | Treat Diff vs. Placebo: p-value (Dif, 95% CI) [1] | | | 0.011 | (−1.6, −2.9 to −0.4) | 0.511 | (−0.4, −1.6 to 0.8) |
| Stage 2 | Stage 2 Baseline: N, Mean (SD) [2] | 40, 18.8 | (7.79) | 40, 18.3 | (8.94) | 44, 18.6 | (8.23) |
| (Stage 1 | Week 9 Change from Baseline: N, Mean (SD) | 40, −0.5 | (3.81) | 40, −1.3 | (4.05) | 44, −1.3 | (3.95) |
| Placebo | Change from Baseline: LS Mean (SE) [1] | −0.5 | (0.59) | −1.3 | (0.59) | −1.2 | (0.56) |
| Non- | Treat Diff vs. Placebo: p-value (Dif, 95% CI) [1] | 39, −0.2 | (3.52) | 0.307 | (−0.9. −2.5 to 0.8) | 0.354 | (−0.8, −2.4 to 0.9) |
| responders | Week 12 Change from Baseline: N, Mean (SD) | −0.1 | (0.61) | 40, −0.7 | (4.03) | 41, −1.8 | (4.41) |
| only) | Change from Baseline: LS Mean (SE) [1] | | | −0.7 | (0.60) | −1.9 | (0.59) |
| | Treat Diff vs. Placebo: p-value (Dif, 95% CI) [1] | | | 0.475 | (−0.6, −2.3 to 1.1) | 0.043 | (−1.7, −3.4 to −0.1) |
| | SPCD Week 6 & 12 MMRM weighted | | | −2.38, p = 0.017 | | -1.87, p = 0.062 | |
| | z-statistic, p-value [3] | | | | | | |

Note:
Factor 2, Physically Non-aggressive Behavior ranges 6 to 42 with higher scores indicating worsening condition.
[1] MMRMs include fixed effects of treatment, visit, treatment-by-visit, baseline, baseline-by-visit, and in the Stage 1 model, baseline NPI AA (≤6 vs. >6), risk assessment for falls (normal/mild vs. moderate/severe), baseline concomitant use of antipsychotic medications (yes vs. no). Unstructured variance-covariance was used. Stage 1 and 2 baseline values were used in their respective models.
[2] Stage 2 Baseline is the last non-missing assessment prior to Stage 2 re-randomization (re-randomization visit).
[3] SPCD Week 6 & 12 MMRM weighted z-statistic was used with weight = 0.6 for Stage 1 and 0.4 for Stage 2.

TABLE 24d
CMAI: Change from Baseline Parallel Group MMRM Analysis-Observed Data (mITT 12-Week Parallel Group Population)
CMAI: Physically Non-Aggressive Behavior

| Parameter/Results | Placebo [1] | | AVP-786 18 mg [1] | | AVP-786 28 mg [1] | |
|---|---|---|---|---|---|---|
| Baseline: N, Mean (SD) | 62, 19.6 | (7.02) | 92, 20.7 | (8.13) | 97, 20.1 | (8.73) |
| Week 1 Change from Baseline: N, Mean (SD) | 60, −1.8 | (4.08) | 88, −2.2 | (4.21) | 94, −1.9 | (3.30) |
| Change from Baseline: LS Mean (SE) [1] | −2.0 | (0.56) | −2.4 | (0.51) | −2.2 | (0.50) |
| Treat Diff vs. Placebo: p-value (Dif, 95% CI) [2] | | | 0.512 | (−0.4, −1.6 to 0.8) | 0.827 | (−0.1, −1.3 to 1.1) |
| Week 2 Change from Baseline: N, Mean (SD) | 48, −2.3 | (4.32) | 70, −2.8 | (4.26) | 70, −1.9 | (3.54) |
| Change from Baseline: LS Mean (SE) [1] | −2.6 | (0.58) | −3.1 | (0.53) | −2.2 | (0.52) |
| Treat Diff vs. Placebo: p-value (Dif, 95% CI) [2] | | | 0.406 | (−0.5, −1.8 to 0.7) | 0.522 | (0.4, −0.9 to 1.7) |
| Week 3 Change from Baseline: N, Mean (SD) | 61, −1.8 | (5.80) | 87, −3.8 | (5.14) | 95, −2.9 | (5.00) |
| Change from Baseline: LS Mean (SE) [1] | −2.3 | (0.69) | −3.9 | (0.61) | −3.3 | (0.59) |
| Treat Diff vs. Placebo: p-value (Dif, 95% CI) [2] | | | 0.050 | (−1.6, −3.2 to 0.0) | 0.198 | (−1.0, −2.6 to 0.5) |
| Week 6 Change from Baseline: N, Mean (SD) | 60, −2.4 | (5.37) | 85, −4.7 | (5.29) | 94, −3.2 | (5.04) |
| Change from Baseline: LS Mean (SE) [1] | −2.8 | (0.69) | −4.8 | (0.61) | −3.7 | (0.59) |
| Treat Diff vs. Placebo: p-value (Dif, 95% CI) [2] | | | 0.015 | (−2.0, −3.6 to −0.4) | 0.281 | (−0.9, −2.4 to 0.7) |
| Week 9 Change from Baseline: N, Mean (SD) | 57, −2.9 | (6.06) | 84, −4.7 | (5.19) | 91, −3.5 | (5.81) |
| Change from Baseline: LS Mean (SE) [1] | −3.3 | (0.73) | −4.9 | (0.64) | −3.8 | (0.62) |
| Treat Diff vs. Placebo: p-value (Dif, 95% CI) [2] | | | 0.056 | (−1.7, −3.4 to 0.0) | 0.528 | (−0.5. −2.2 to 1.1) |
| Week 12 Change from Baseline: N, Mean (SD) | 57, −2.6 | (5.75) | 85, −5.3 | (5.58) | 92, −3.9 | (5.49) |
| Change from Baseline: LS Mean (SE) [1] | −3.0 | (0.73) | −5.5 | (0.63) | −4.2 | (0.62) |
| Treat Diff vs. Placebo: p-value (Dif, 95% CI) [2] | | | 0.003 | (−2.5, −4.2 to −0.8) | 0.147 | (−1.2, −2.9 to 0.4) |

Note:
Factor 2, Physically Non-aggressive Behavior ranges 6 to 42 with higher scores Indicating worsening condition.
[1] Patients who are randomized to the same treatment group in both stages.
[2] MMRM with fixed effects of treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no). Unstructured variance-covariance was used.

TABLE 24e
CMAI: Change from Baseline SPCD MMRM - Observed Data (mITT Population)
CMAI: Verbally Agitated Behavior

| Stage | Parameter/Results | Placebo | | AVP-786 18 mg | | AVP-786 28 mg | |
|---|---|---|---|---|---|---|---|
| Stage 1 | Baseline: N, Mean (SD) | 188, 17.6 | (5.91) | 92, 16.7 | (6.11) | 97, 17.1 | (5.42) |
| | Week 1 Change from Baseline: N, Mean (SD) | 182, −1.0 | (3.57) | 88, −1.1 | (3.76) | 94, −1.7 | (3.39) |
| | Change from Baseline: LS Mean (SE) [1] | −1.0 | (0.34) | −1.2 | (0.43) | −1.7 | (0.42) |
| | Treat Diff vs. Placebo: p-value (Dif, 95% CI) [1] | 141, −1.4 | (3.54) | 0.684 | (−0.2, −1.0 to 0.7) | 0.112 | (−0.7, −1.5 to 0.2) |
| | Week 2 Change from Baseline: N, Mean (SD) | −1.3 | (0.35) | 70, −2.1 | (3.99) | 70, −1.6 | (3.19) |
| | Change from Baseline: LS Mean (SE) [1] | 184, −2.2 | (4.55) | −2.0 | (0.44) | −1.6 | (0.44) |
| | Treat Diff vs. Placebo: p-value (Dif, 95% CI) [1] | −2.1 | (0.38) | 0.124 | (−0.7, −1.6 to 0.2) | 0.483 | (−0.3, −1.2 to 0.6) |
| | Week 3 Change from Baseline: N, Mean (SD) | 180, −2.4 | (4.34) | 87, −2.3 | (4.60) | 95, −2.0 | (4.22) |
| | Change from Baseline: LS Mean (SE) [1] | −2.2 | (0.38) | −2.5 | (0.50) | −2.0 | (0.48) |
| | Treat Diff vs. Placebo: p-value (Dif, 95% CI) [1] | | | 0.476 | (−0.4, −1.4 to 0.7) | 0.772 | (0.2, −0.9 to 1.2) |
| | Week 6 Change from Baseline: N, Mean (SD) | | | 85, −2.7 | (4.66) | 94, −2.4 | (4.60) |
| | Change from Baseline: LS Mean (SE) [1] | | | −3.0 | (0.50) | −2.5 | (0.48) |
| | Treat Diff vs. Placebo: p-value (Dif, 95% CI) [1] | | | 0.145 | (−0.8, −1.8 to 0.3) | 0.571 | (−0.3, −1.3 to 0.7) |
| Stage 2 | Stage 2 Baseline: N, Mean (SD) [2] | 40, 15.2 | (5.75) | 40, 17.7 | (5.29) | 44, 15.7 | (6.23) |
| (Stage 1 | Week 9 Change from Baseline: N, Mean (SD) | 40, −0.1 | (3.11) | 40, −1.4 | (3.73) | 44, −0.4 | (3.01) |
| Placebo | Change from Baseline: LS Mean (SE) [1] | −0.3 | (0.51) | −1.2 | (0.51) | −0.5 | (0.48) |
| Non- | Treat Diff vs. Placebo: p-value (Dif, 95% CI) [1] | 39, −0.8 | (3.92) | 0.212 | (−0.9, −2.3 to 0.5) | 0.759 | (−0.2, −1.6 to 1.2) |
| responders | Week 12 Change from Baseline: N, Mean (SD) | −0.9 | (0.59) | 40, −2.4 | (3.69) | 41, −1.3 | (3.91) |
| only) | Change from Baseline: LS Mean (SE) [1] | | | −2.1 | (0.59) | −1.3 | (0.58) |
| | Treat Diff vs. Placebo: p-value (Dif, 95% CI) [1] | | | 0.165 | (−1.2, −2.9 to 0.5) | 0.657 | (−0.4, −2.0 to 1.3) |
| | SPCD Week 6 & 12 MMRM weighted | | | −2.02, p = 0.044 | | −0.71, p = 0.476 | |
| | z-statistic, p-value [3] | | | | | | |

Note:
Factor 3, Verbally Agitated Behavior ranges 4 to 28 with higher scores indicating worsening condition.
[1] MMRMs include fixed effects of treatment, visit, treatment-by-visit, baseline, baseline-by-visit, and in the Stage 1 model, baseline NPI AA (≤6 vs. >6), risk assessment for falls (normal/mild vs. moderate/severe), baseline concomitant use of antipsychotic medications (yes vs. no). Unstructured variance-covariance was used. Stage 1 and 2 baseline values were used in their respective models.
[2] Stage 2 Baseline is the last non-missing assessment prior to Stage 2 re-randomization (re-randomization visit).
[3] SPCD Week 6 & 12 MMRM weighted z-statistic was used with weight = 0.6 for Stage 1 and 0.4 for Stage 2.

TABLE 24f
CMAI: Change from Baseline Parallel Group MMRM Analysis-Observed Data (mITT 12-Week Parallel Group Population)
CMAI: Verbally Agitated Behavior

| Parameter/Results | Placebo [1] | | AVP-786 18 mg [1] | | AVP-786 28 mg [1] | |
|---|---|---|---|---|---|---|
| Baseline: N, Mean (SD) | 62, 17.0 | (5.79) | 92, 16.7 | (6.11) | 97, 17.1 | (5.42) |
| Week 1 Change from Baseline: N, Mean (SD) | 60, −1.2 | (3.24) | 88, −1.1 | (3.76) | 94, −1.7 | (3.39) |
| Change from Baseline: LS Mean (SE) [1] | −1.1 | (0.50) | −1.1 | (0.45) | −1.6 | (0.44) |
| Treat Diff vs. Placebo: p-value (Dif, 95% CI) [2] | | | 0.967 | (−0.0, −1.1 to 1.1) | 0.352 | (−0.5, −1.6 to 0.6) |
| Week 2 Change from Baseline: N, Mean (SD) | 48, −0.9 | (3.29) | 70, −2.1 | (3.99) | 70, −1.6 | (3.19) |
| Change from Baseline: LS Mean (SE) [1] | −1.0 | (0.51) | −2.0 | (0.45) | −1.6 | (0.45) |
| Treat Diff vs. Placebo: p-value (Dif, 95% CI) [2] | | | 0.089 | (−1.0. −2.1 to 0.1) | 0.321 | (−0.6, −1.7 to 0.5) |
| Week 3 Change from Baseline: N, Mean (SD) | 61, −2.3 | (5.17) | 87, −2.3 | (4.60) | 95, −2.0 | (4.22) |
| Change from Baseline: LS Mean (SE) [1] | −2.3 | (0.60) | −2.4 | (0.53) | −1.9 | (0.51) |
| Treat Diff vs. Placebo: p-value (Dif, 95% CI) [2] | | | 0.834 | (−0.1, −1.5 to 1.2) | 0.560 | (0.4, −1.0 to 1.8) |
| Week 6 Change from Baseline: N, Mean (SD) | 60, −2.3 | (4.78) | 85, −2.7 | (4.66) | 94, −2.4 | (4.60) |
| Change from Baseline: LS Mean (SE) [1] | −2.2 | (0.60) | −2.9 | (0.53) | −2.4 | (0.51) |
| Treat Diff vs. Placebo: p-value (Dif, 95% CI) [2] | | | 0.350 | (−0.7. −2.1 to 0.7) | 0.780 | (−0.2, −1.6 to 1.2) |
| Week 9 Change from Baseline: N, Mean (SD) | 57, −2.4 | (5.20) | 84, −3.1 | (5.16) | 91, −2.7 | (4.89) |
| Change from Baseline: LS Mean (SE) [1] | −2.5 | (0.65) | −3.2 | (0.57) | −2.7 | (0.55) |
| Treat Diff vs. Placebo: p-value (Dif, 95% CI) [2] | | | 0.357 | (−0.7, −2.2 to 0.8) | 0.818 | (−0.2, −1.7 to 1.3) |
| Week 12 Change from Baseline: N, Mean (SD) | 57, −3.1 | (5.26) | 85, −3.2 | (4.76) | 92, −2.8 | (4.64) |
| Change from Baseline: LS Mean (SE) [1] | −2.9 | (0.64) | −3.4 | (0.56) | −2.8 | (0.54) |
| Treat Diff vs. Placebo: p-value (Dif, 95% CI) [2] | | | 0.549 | (−0.5, −2.0 to 1.0) | 0.915 | (0.1, −1.4 to 1.6) |

Note:
Factor 3, Verbally Agitated Behavior ranges 4 to 28 with higher scores indicating worsening condition.
[1] Patients who are randomized to the same treatment group in both stages.
[2] MMRM with fixed effects of treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no). Unstructured variance-covariance was used.

TABLE 25
CMAI Subscale Scores: Change from Baseline Parallel Group MMRM
Analysis-Observed Data (mITT 12-week Parallel Group Population)

| Parameter/Results | Placebo [1] | | AVP-786-18 [1] | | AVP-786-28 [1] | |
|---|---|---|---|---|---|---|
| CMAI: F1-Aggressive Behavior | | | | | | |
| Baseline: N, Mean (SD) | 62, 21.0 | (10.14) | 92, 21.2 | (8.86) | 97, 20.7 | (8.70) |
| Week 12 Change from Baseline: LS Mean (SE) [2] | −3.3 | (0.79) | −4.8 | (0.70) | −3.8 | (0.68) |
| Treat Diff vs. Placebo: p-value (Dif, 95% CI) [2] | | | 0.1 17 | (−1.5, −3.3 to 0.4) | 0.646 | (−0.4, −2.2 to 1.4) |
| CMAI: F2-Physically Nonaggressive Behavior | | | | | | |
| Baseline: N, Mean (SD) | 62, 19.6 | (7.02) | 92, 20.7 | (8.13) | 97, 20.1 | (8.73) |
| Week 12 Change from Baseline: LS Mean (SE) [2] | −3.0 | (0.73) | −5.5 | (0.63) | −4.2 | (0.62) |
| Treat Diff vs. Placebo: p-value (Dif, 95% CI) [2] | | | 0.003 | (−2.5, −4.2 to −0.8) | 0.147 | (−1.2, −2.9 to 0.4) |
| CMAI: F3-Verbally Agitated Behavior | | | | | | |
| Baseline: N, Mean (SD) | 62, 17.0 | (5.79) | 92, 16.7 | (6.11) | 97, 17.1 | (5.42) |
| Week 12 Change from Baseline: LS Mean (SE) [2] | −2.9 | (0.64) | −3.4 | (0.56) | −2.8 | (0.54) |
| Treat Diff vs. Placebo: p-value (Dif, 95% CI) [2] | | | 0.549 | (−0.5, −2.0 to 1.0) | 0.915 | (0.1, −1.4 to 1.6) |

Note:
Factor 1, Aggressive Behavior ranges from 12 to 84. Factor 2, F2-Physically Nonaggressive Behavior ranges 6 to 42. Factor 3, F3-Vetbally Agitated Behavior ranges 4 to 28. For all factors, higher scoies indicate worsening condition.
[1] Patients who are randomized to the same treatment group in both stages.
[2] MMRM with fixed effects of treatment, visit, treatment-by-visit. Baseline, Baseline-by-visit, Baseline NPI AA (≤6 vs. >6), risk assessment for falls (normal/mild vs. moderate/severe). Baseline concomitant use of antipsychotic medications (yes vs. no). Unstructured variance-covariance was used.

### CMAI F1—Aggressive Behavior

In the SPCD analysis, patients treated with AVP-786-18 showed greater improvement in the mean CMAI F1—Aggressive Behavior score compared with placebo (significant at the nominal level, p=0.018; Table 24, above, Table 24a, above). For patients treated with AVP-786-28 compared with placebo, the p-value was not significant at the nominal level (p=0.292).

In Stage 1, patients treated with AVP-786-18 showed greater change in the mean CMAI F1—Aggressive Behavior score compared with placebo (treatment difference [CI]: −1.2 [−2.4 to −0.0]), which was significant at the nominal level (p=0.047). The treatment difference (CI) for patients treated with AVP-786-28 compared with placebo was −0.2 (−1.4 to 1.0) with a p=0.731 (Table 24, above).

In Stage 2, patients treated with AVP-786-18 and AVP-786-28 showed greater change in the mean CMAI F1—Aggressive Behavior score compared with placebo (treatment difference [CI]: (−1.5 [−3.7 to 0.6] and −1.2 [−3.3 to 1.0], respectively; Table 24, above), which did not reach significance at the nominal level (p=0.158 and p=0.280, respectively).

The change from Baseline in the mean CMAI F1—Aggressive Behavior score is presented for the 12-week Parallel Group in Table 24b, above, and in Table 25. The treatment differences (CI) for patients treated with AVP-786-18 and AVP-786-28 versus placebo were −1.5 (−3.3 to 0.4) and −0.4 (−2.2 to 1.4), respectively, with p=0.117 and p=0.646.

### CMAI F2—Physically Nonaggressive Behavior

In the SPCD analysis, patients treated with AVP-786-18 showed greater change in the mean CMAI F2—Physically Nonaggressive Behavior score compared with placebo (significant at the nominal level, p=0.017; Table 24, above, Table 24c, above). For patients treated with AVP-786-28 compared with placebo, the p-value was not significant at the nominal level (p=0.062).

In Stage 1, patients treated with AVP-786-18 showed greater change in the mean CMAI F2—Physically Nonaggressive Behavior score compared with placebo (treatment difference [CI]: −1.6 [−2.9 to −0.4]), which was significant at the nominal level (p=0.011). The treatment difference (CI) for patients treated with AVP-786-28 compared with placebo was −0.4 (−1.6 to 0.8), with a p=0.511.

In Stage 2, patients treated with AVP-786-18 and AVP-786-28 showed greater change in the mean CMAI F2—Physically Nonaggressive Behavior score compared with placebo (treatment difference [CI]: (−0.6 [−2.3 to 1.1] and −1.7 [−3.4 to −0.1], respectively); the difference did not reach significance at the nominal level for AVP-786-18 (p=0.475) but was significant for AVP-786-28 (p=0.043).

The change from Baseline in the mean CMAI F2—Physically Nonaggressive Behavior score is presented for the 12-week Parallel Group in Table 24d, above, and in Table 25. Patients treated with AVP-786-18 showed greater improvement in the mean CMAI F2—Physically Nonaggressive Behavior score compared with placebo (treatment difference [CI]: −2.5 [−4.2 to −0.8]), which was significant at the nominal level (p=0.003). The treatment difference (CI) for patients treated with AVP-786-28 compared with placebo was −1.2 (−2.9 to 0.4), with a p=0.147 (Table 25).

### CMAI F3—Verbally Agitated Behavior

In the SPCD analysis, patients treated with AVP-786-18 showed greater change in the mean CMAI F3—Verbally Agitated Behavior score compared with placebo (significant at the nominal level, p=0.044; Table 24e, above, and Table 24). For patients treated with AVP-786-28 compared with placebo, the p-value was not significant at the nominal level (p=0.476).

In Stage 1, patients treated with AVP-786-18 showed greater change in the mean CMAI F3—Verbally Agitated Behavior score compared with placebo (treatment difference [CI]: −0.8 [−1.8 to 0.3]), which did not reach significance at the nominal level (p=0.145). The treatment difference (CI) for patients treated with AVP-786-28 compared with placebo was −0.3 (−1.3 to 0.7) with p=0.571 (Table 24).

In Stage 2, patients treated with AVP-786-18 showed greater change in the mean CMAI F3—Verbally Agitated Behavior score compared with placebo (treatment difference [CI]: −1.2 [−2.9 to 0.5]), which did not reach significance at the nominal level (p=0.165; Table 24). The treatment difference (CI) for patients treated with AVP-786-28 compared with placebo was −0.4 (−2.0 to 1.3) with p=0.657.

The change from Baseline in the mean CMAI F3—Verbally Agitated Behavior score is presented for the 12-week Parallel Group in Table 24f, above, and in Table 25. The treatment differences (CI) for patients treated with AVP-786-18 and AVP-786-28 versus placebo were −0.5 (−2.0 to 1.0) and 0.1 (−1.4 to 1.6), respectively, with p=0.549 and p=0.915.

CMAI Total Score—Change from Baseline in Patient Sub-Groups

Tables 26A-26VV below show changes from baseline in CMAI Total score in the patient subgroups identified in each Table:

TABLE 26A
CMAI Total Score: Change from Baseline SPCD MMRM, Observed Data
By Gender
mITT Population and CMAI Factor 1 Agitated at Baseline
Male

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 63, 72.9 | (19.44) | 31, 76.1 | (24.60) | 32, 71.9 | (22.86) |
| Week 1 Change from Baseline: N, Mean (SD) | 61, −6.7 | (8.81) | 29, −9.5 | (15.02) | 31, −10.4 | (11.66) |
| Week 1 Change from Baseline: LSMean (SE) | −5.3 | (2.01) | −8.3 | (2.47) | −9.5 | (2.32) |
| TRT Diff vs. PBO: p-value (Diff, 95% CI) | | | 0.224 (−2.98, −7.80 to 1.85) | | 0.084 (−4.14, −8.83 to 0.56) | |
| Week 2 Change from Baseline: N, Mean (SD) | 49, −7.9 | (10.55) | 20, −14.8 | (18.49) | 23, −9.9 | (12.26) |
| Week 2 Change from Baseline: LSMean (SE) | −6.1 | (2.08) | −12.5 | (2.63) | −8.5 | (2.46) |
| TRT Diff vs. PBO: p-value (Diff, 95% CI) | | | 0.018 (−6.35, −11.59 to −1.11) | | 0.343 (−2.44, −7.50 to 2.63) | |
| Week 3 Change from Baseline: N, Mean (SD) | 63, −10.3 | (13.44) | 29, −17.9 | (18.82) | 32, −9.9 | (13.94) |
| Week 3 Change from Baseline: LSMean (SE) | −9.3 | (2.27) | −15.4 | (2.92) | −8.4 | (2.75) |
| TRT Diff vs. PBO: p-value (Diff, 95% CI) | | | 0.052 (−6.05, −12.15 to 0.05) | | 0.760 (0.91, −4.99 to 6.31) | |
| Week 6 Change from Baseline: N, Mean (SD) | 61, −11.5 | (14.58) | 27, −18.6 | (19.42) | 31, −12.0 | (14.72) |
| Week 6 Change from Baseline: LSMean (SE) | −10.6 | (2.25) | −16.3 | (2.90) | −11.9 | (2.72) |
| TRT Diff vs. PBO: p-value (Diff, 95% CI) | | | 0.066 (−5.66, −11.70 to 0.38) | | 0.671 (−1.25, −7.06 to 4.56) | |
| Stage 2 (Stage 1 Placebo Non-Responder Only) | | | | | | |
| Baseline: N, Mean (SD) | 11, 63.3 | (18.64) | 13, 63.5 | (20.99) | 16, 60.5 | (14.54) |
| Week 9 Change from Baseline: N, Mean (SD) | 11, −0.3 | (6.80) | 13, −4.3 | (11.97) | 16, −2.4 | (9.43) |
| Week 9 Change from Baseline: LSMean (SE) | 0.7 | (2.80) | −4.3 | (2.55) | −3.0 | (2.31) |
| TRT Diff vs. PBO: p-value (Diff, 95% CI) | | | 0.196 (−4.99, −12.66 to 2.68) | | 0.312 (−3.67, −11.08 to 3.74) | |
| Week 12 Change from Baseline: N, Mean (SD) | 11, −3.9 | (11.03) | 13, 0.0 | (15.73) | 16, −4.3 | (11.99) |
| Week 12 Change from Baseline: LSMean (SE) | −2.5 | (3.71) | −0.0 | (3.38) | −5.1 | (3.07) |

| | | |
|---|---|---|
| TRT Diff vs. PBO: p-value (Diff, 95% CI) | 0.625 (2.47, −7.71 to 12.65) | 0.589 (−2.64, −12.48 to 7.19) |
| OLS Z-statistic, p-value | −0.886, p = 0.375 | −0.690, p = 0.490 |

TABLE 26B
CMAI Total Score: Change from Baseline SPCD MMRM, Observed Data
By Gender
mITT Population and CMAI Factor 1 Agitated at Baseline
Female

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 80, 83.0 | (24.03) | 33, 83.8 | (19.90) | 34, 86.5 | (23.65) |
| Week 1 Change from Baseline: N, Mean (SD) | 78, −7.4 | (10.97) | 33, −7.2 | (12.80) | 33, −6.2 | (8.63) |
| Week 1 Change from Baseline: LSMean (SE) | −8.9 | (2.01) | −8.8 | (2.45) | −7.2 | (2.45) |
| TRT Diff vs. PBO: p-value (Diff, 95% CI) | | | 0.951 (0.14, −4.26 to 4.54) | | 0.443 (1.70, −2.68 to 6.08) | |
| Week 2 Change from Baseline: N, Mean (SD) | 66, −9.5 | (12.36) | 29, −9.7 | (14.76) | 27, −7.7 | (10.15) |
| Week 2 Change from Baseline: LSMean (SE) | −11.8 | (2.10) | −11.3 | (2.64) | −8.6 | (2.65) |
| TRT Diff vs. PBO: p-value (Diff, 95% CI) | | | 0.851 (0.48, −4.52 to 5.47) | | 0.227 (3.05, −1.93 to 8.03) | |
| Week 3 Change from Baseline: N, Mean (SD) | 77, −12.5 | (14.62) | 33, −12.9 | (16.14) | 34, −12.7 | (12.32) |
| Week 3 Change from Baseline: LSMean (SE) | −13.9 | (2.22) | −14.6 | (2.86) | −13.6 | (2.96) |
| TRT Diff vs. PBO: p-value (Diff, 95% CI) | | | 0.814 (−0.68, −6.33 to 4.98) | | 0.921 (0.32, −5.28 to 5.92) | |
| Week 6 Change from Baseline: N, Mean (SD) | 76, −15.5 | (15.30) | 32, −16.1 | (16.00) | 33, −14.3 | (12.72) |
| Week 6 Change from Baseline: LSMean (SE) | −16.7 | (2.27) | −13.0 | (2.97) | −15.3 | (2.94) |
| TRT Diff vs. PBO: p-value (Diff, 95% CI) | | | 0.869 (−1.28, −7.18 to 4.63) | | 0.615 (1.49, −4.36 to 7.33) | |
| Stage 2 (Stage 1 Placebo Non-Responder Only) | | | | | | |
| Baseline: N, Mean (SD) | 19, 70.9 | (30.84) | 17, 75.3 | (22.20) | 19, 72.4 | (22.78) |
| Week 9 Change from Baseline: N, Mean (SD) | 18, 0.6 | (7.83) | 17, −7.3 | (8.74) | 19, −3.6 | (12.02) |
| Week 9 Change from Baseline: LSMean (SE) | 0.4 | (2.20) | −7.0 | (2.26) | −3.7 | (2.14) |
| TRT Diff vs. PBO: p-value (Diff, 95% CI) | | | 0.024 (−7.36, −13.70 to −1.01) | | 0.192 (−4.06, −10.21 to 2.10) | |
| Week 12 Change from Baseline: N, Mean (SD) | 18, 0.5 | (9.97) | 17, −9.7 | (10.07) | 18, −8.6 | (13.61) |
| Week 12 Change from Baseline: LSMean (SE) | 0.3 | (2.63) | −9.5 | (2.71) | −8.6 | (2.60) |

| | | |
|---|---|---|
| TRT Diff vs. PBO: p-value (Diff, 95% CI) | 0.013 (−9.80, −17.40 to −2.19) | 0.067 (−6.92, −14.36 to 0.51) |
| OLS Z-statistic, p-value | −1.997, p = 0.046 | −0.912, p = 0.417 |

TABLE 26C
CMAI: Change from Baseline Parallel Group MMRM Analysis - Observed Data
By Gender
mITT 12-Week Parallel Group Population and CMAI Factor 1 Agitated at Baseline
Male

| Parameter/Result | Placebo | | AVP-786 18 mg | | AVP-786 28 mg | |
|---|---|---|---|---|---|---|
| Baseline: N, Mean (SD) | 19, 69.4 | (14.99) | 31, 76.1 | (24.60) | 32, 71.9 | (21.86) |
| Week 1 Change from Baseline: N, Mean (SD) | 17, −9.8 | (7.41) | 29, −9.5 | (15.02) | 31, −10.4 | (11.66) |
| Week 1 Change from Baseline: LSMean (SE) | −6.1 | (3.17) | −7.2 | (2.69) | −8.2 | (2.59) |
| TRT Diff vs. PBO: p-value (Diff, 95% CI) | | | 0.759 (−1.10, −8.15 to 5.95) | | 0.532 (−2.17, −9.07 to 4.92) | |
| Week 2 Change from Baseline: N, Mean (SD) | 16, −7.0 | (10.06) | 28, −14.3 | (18.49) | 23, −9.9 | (12.26) |
| Week 2 Change from Baseline: LSMean (SE) | −5.2 | (3.29) | −11.8 | (2.89) | −7.3 | (2.69) |
| TRT Diff vs. PBO: p-value (Diff, 95% CI) | | | 0.095 (−6.62, −14.13 to 0.94) | | 0.532 (−2.04, −9.39 to 5.32) | |
| Week 3 Change from Baseline: N, Mean (SD) | 19, −8.3 | (15.30) | 28, −17.9 | (18.82) | 32, −8.9 | (13.94) |
| Week 3 Change from Baseline: LSMean (SE) | −7.2 | (3.70) | −14.0 | (3.14) | −7.1 | (2.94) |
| TRT Diff vs. PBO: p-value (Diff, 95% CI) | | | 0.125 (−6.73, −15.38 to 1.91) | | 0.971 (0.15, −3.26 to 8.57) | |
| Week 6 Change from Baseline: N, Mean (SD) | 18, −8.2 | (15.08) | 27, −18.6 | (19.42) | 31, −12.0 | (14.72) |
| Week 6 Change from Baseline: LSMean (SE) | −7.2 | (3.64) | −15.1 | (3.09) | −10.4 | (2.89) |
| TRT Diff vs. PBO: p-value (Diff, 95% CI) | | | 0.068 (−7.87, −16.35 to 0.60) | | 0.437 (−9.23, −11.49 to 5.02) | |
| Week 9 Change from Baseline: N, Mean (SD) | 17, −8.9 | (16.96) | 27, −15.6 | (33.20) | 29, −11.6 | (15.65) |
| Week 9 Change from Baseline: LSMean (SE) | −8.3 | (4.23) | −13.2 | (3.52) | −10.4 | (3.31) |
| TRT Diff vs. PBO: p-value (Diff, 95% CI) | | | 0.342 (−4.94, −14.91 to 5.23) | | 0.673 (−2.09, −11.93 to 7.74) | |
| Week 12 Change from Baseline: N, Mean (SD) | 17, −9.4 | (12.09) | 27, −17.6 | (19.57) | 31, −9.3 | (12.38) |
| Week 12 Change from Baseline: LSMean (SE) | −8.6 | (3.45) | −15.0 | (2.92) | −8.2 | (2.72) |

| | | |
|---|---|---|
| TRT Diff vs. PBO: p-value (Diff, 95% CI) | 0.108 (−6.43, −14.40 to 1.45) | 0.925 (0.96, −7.32 to 8.05) |

TABLE 26D
CMAI: Change from Baseline Parallel Group MMRM Analysis - Observed Data
By Gender
mITT 12-Week Parallel Group Population and CMAI Factor 1 Agitated at Baseline
Female

| Parameter/Result | Placebo | | AVP-786 18 mg | | AVP-786 28 mg | |
|---|---|---|---|---|---|---|
| Baseline: N, Mean (SD) | 27, 80.6 | (26.00) | 33, 83.8 | (19.90) | 34, 86.5 | (23.65) |
| Week 1 Change from Baseline: N, Mean (SD) | 27, −7.5 | (10.78) | 33, −7.2 | (12.80) | 33, −6.2 | (8.63) |
| Week 1 Change from Baseline: LSMean (SE) | −11.5 | (2.77) | −10.9 | (2.67) | −9.5 | (2.69) |
| TRT Diff vs. PBO: p-value (Diff, 95% CI) | | | 0.839 (0.57, −4.93 to 6.12) | | 0.493 (1.96, −3.56 to 7.49) | |
| Week 2 Change from Baseline: N, Mean (SD) | 22, −8.5 | (14.19) | 29, −9.7 | (14.76) | 27, −7.7 | (10.15) |
| Week 2 Change from Baseline: LSMean (SE) | −13.8 | (3.00) | −13.4 | (2.85) | −10.8 | (2.37) |
| TRT Diff vs. PBO: p-value (Diff, 95% CI) | | | 0.947 (0.31, −6.14 to 6.57) | | 0.384 (2.79, −3.54 to 9.12) | |
| Week 3 Change from Baseline: N, Mean (SD) | 26, −12.0 | (16.98) | 33, −12.9 | (16.14) | 34, −12.7 | (13.92) |
| Week 3 Change from Baseline: LSMean (SE) | −15.4 | (3.39) | −16.7 | (3.19) | −15.8 | (3.19) |
| TRT Diff vs. PBO: p-value (Diff, 95% CI) | | | 0.795 (−1.30, −8.90 to 6.31) | | 0.923 (−0.37, −7.93 to 7.19) | |
| Week 6 Change from Baseline: N, Mean (SD) | 26, −15.1 | (15.71) | 32, −16.1 | (16.00) | 33, −14.3 | (12.72) |
| Week 6 Change from Baseline: LSMean (SE) | −18.5 | (3.35) | −20.1 | (3.16) | −17.7 | (3.15) |
| TRT Diff vs. PBO: p-value (Diff, 95% CI) | | | 0.674 (−1.59, −9.09 to 5.91) | | 0.824 (0.84, −6.61 to 8.28) | |
| Week 9 Change from Baseline: N, Mean (SD) | 25, −14.2 | (16.73) | 31, −20.9 | (14.96) | 33, −14.2 | (15.23) |
| Week 9 Change from Baseline: LSMean (SE) | −18.2 | (3.44) | −24.8 | (3.23) | −17.6 | (3.21) |
| TRT Diff vs. PBO: p-value (Diff, 95% CI) | | | 0.091 (−6.70, −14.43 to 1.09) | | 0.888 (0.55, −7.17 to 8.26) | |
| Week 12 Change from Baseline: N, Mean (SD) | 26, −15.0 | (20.61) | 32, −20.4 | (18.00) | 33, −18.0 | (15.69) |
| Week 12 Change from Baseline: LSMean (SE) | −18.6 | (3.90) | −24.4 | (3.63) | −21.3 | (3.61) |

| | | |
|---|---|---|
| TRT Diff vs. PBO: p-value (Diff, 95% CI) | 0.209 (−5.86, −15.08 to 3.33) | 0.550 (−2.76, −11.89 to 6.38) |

TABLE 26E
CMAI Total Score: Change from Baseline SPCD MMRM, Observed Data
By Age Group (<75, >=75)
mITT Population and CMAI Factor 1 Agitated at Baseline
Age <75 Years

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 50, 80.3 | (24.53) | 32, 81.9 | (20.05) | 33, 70.6 | (21.92) |
| Week 1 Change from Baseline: N, Mean (SD) | 47, −5.3 | (8.37) | 32, −6.7 | (13.76) | 32, −8.4 | (11.86) |
| Week 1 Change from Baseline: LSMean (SE) | −1.4 | (2.35) | −3.2 | (2.49) | −6.0 | (2.62) |
| TRT Diff vs. PBO: p-value (Diff, 95% CI) | | | 0.471 (−1.75, −6.53 to 3.04) | | 0.062 (−4.61, −9.46 to 0.24) | |
| Week 2 Change from Baseline: N, Mean (SD) | 45, −5.5 | (9.46) | 24, −11.6 | (18.58) | 25, −6.9 | (13.08) |
| Week 2 Change from Baseline: LSMean (SE) | −1.7 | (2.49) | −8.4 | (2.77) | −5.4 | (2.87) |
| TRT Diff vs. PBO: p-value (Diff, 95% CI) | | | 0.019 (−6.78, −13.40 to −1.17) | | 0.189 (−3.78, −9.49 to 1.87) | |
| Week 3 Change from Baseline: N, Mean (SD) | 43, −3.9 | (12.51) | 32, −14.6 | (19.01) | 33, −10.2 | (13.57) |
| Week 3 Change from Baseline: LSMean (SE) | −4.8 | (2.71) | −10.8 | (3.09) | −8.6 | (3.12) |
| TRT Diff vs. PBO: p-value (Diff, 95% CI) | | | 0.070 (−5.96, −12.42 to 0.51) | | 0.254 (−3.76, −10.27 to 2.74) | |
| Week 6 Change from Baseline: N, Mean (SD) | 47, −12.1 | (13.36) | 31, −16.9 | (18.65) | 32, −12.7 | (14.43) |
| Week 6 Change from Baseline: LSMean (SE) | −7.9 | (2.71) | −13.2 | (3.02) | −11.5 | (3.12) |
| TRT Diff vs. PBO: p-value (Diff, 95% CI) | | | 0.105 (−5.32, −11.77 to 1.13) | | 0.273 (−3.61, −10.10 to 2.89) | |
| Stage 2 (Stage 1 Placebo Non-Responder Only) | | | | | | |
| Baseline: N, Mean (SD) | 13, 75.9 | (34.45) | 11, 67.6 | (21.16) | 9, 62.8 | (21.00) |
| Week 9 Change from Baseline: N, Mean (SD) | 13, −0.5 | (5.77) | 11, −5.7 | (6.90) | 9, −3.1 | (7.56) |
| Week 9 Change from Baseline: LSMean (SE) | 0.1 | (1.76) | −5.9 | (1.89) | −3.7 | (2.11) |
| TRT Diff vs. PBO: p-value (Diff, 95% CI) | | | 0.029 (−5.96, −11.26 to −0.66) | | 0.181 (−3.80, −9.47 to 1.87) | |
| Week 12 Change from Baseline: N, Mean (SD) | 13, −1.3 | (12.48) | 11, −4.4 | (11.41) | 9, −8.8 | (11.04) |
| Week 12 Change from Baseline: LSMean (SE) | −1.0 | (3.35) | −4.5 | (3.60) | −9.1 | (4.01) |

| | | |
|---|---|---|
| TRT Diff vs. PBO: p-value (Diff, 95% CI) | 0.483 (−3.46, −13.55 to 6.63) | 0.195 (−8.12, −18.92 to 2.68) |
| OLS Z-statistic, p-value | −1.649, p = 0.099 | −1.876, p = 0.061 |

TABLE 26F
CMAI Total Score: Change from Baseline SPCD MMRM, Observed Data
By Age Group (<75, >=75)
mITT Population and CMAI Factor 1 Agitated at Baseline
Age >=75 Years

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 93, 77.6 | (21.60) | 32, 78.3 | (24.84) | 33, 88.2 | (22.52) |
| Week 1 Change from Baseline: N, Mean (SD) | 92, −8.0 | (10.74) | 30, −9.9 | (13.91) | 32, −8.0 | (8.77) |
| Week 1 Change from Baseline: LSMean (SE) | −10.5 | (1.75) | −12.9 | (2.38) | −9.8 | (2.23) |
| TRT Diff vs. PBO: p-value (Diff, 95% CI) | | | 0.309 (−2.30, −6.74 to 2.15) | | 0.468 (1.63, −2.90 to 6.06) | |
| Week 2 Change from Baseline: N, Mean (SD) | 69, −11.0 | (12.41) | 25, −12.0 | (14.40) | 25, −10.5 | (8.61) |
| Week 2 Change from Baseline: LSMean (SE) | −13.7 | (1.79) | −14.3 | (2.44) | −9.2 | (2.93) |
| TRT Diff vs. PBO: p-value (Diff, 95% CI) | | | 0.792 (−0.65, −5.31 to 4.00) | | 0.063 (4.45, −0.24 to 9.13) | |
| Week 3 Change from Baseline: N, Mean (SD) | 92, −12.9 | (14.74) | 29, −15.9 | (15.86) | 33, −11.5 | (12.93) |
| Week 3 Change from Baseline: LSMean (SE) | −15.8 | (1.91) | −18.1 | (2.74) | −11.1 | (2.57) |
| TRT Diff vs. PBO: p-value (Diff, 95% CI) | | | 0.405 (−2.28, −7.68 to 3.12) | | 0.084 (4.69, −0.64 to 10.02) | |
| Week 6 Change from Baseline: N, Mean (SD) | 90, −14.6 | (15.98) | 28, −17.5 | (16.54) | 32, −13.6 | (13.07) |
| Week 6 Change from Baseline: LSMean (SE) | −17.4 | (1.98) | −20.3 | (2.91) | −13.2 | (2.72) |
| TRT Diff vs. PBO: p-value (Diff, 95% CI) | | | 0.322 (−2.93, −8.76 to 2.90) | | 0.149 (4.19, −1.52 to 9.90) | |
| Stage 2 (Stage 1 Placebo Non-Responder Only) | | | | | | |
| Baseline: N, Mean (SD) | 16, 65.1 | (17.51) | 19, 71.7 | (23.09) | 26, 69.4 | (20.01) |
| Week 9 Change from Baseline: N, Mean (SD) | 16, 0.9 | (8.54) | −8.2 | (11.86) | 26, −3.0 | (11.82) |
| Week 9 Change from Baseline: LSMean (SE) | 0.3 | (2.61) | −5.5 | (2.40) | −3.0 | (2.04) |
| TRT Diff vs. PBO: p-value (Diff, 95% CI) | | | 0.110 (−5.78, −12.91 to 1.34) | | 0.322 (−3.31, −9.95 to 3.39) | |
| Week 12 Change from Baseline: N, Mean (SD) | 16, −1.0 | (8.81) | 19, −6.2 | (14.84) | 25, −4.3 | (13.19) |
| Week 12 Change from Baseline: LSMean (SE) | −1.9 | (2.90) | −5.2 | (2.66) | −4.4 | (2.30) |

| | | |
|---|---|---|
| TRT Diff vs. PBO: p-value (Diff, 95% CI) | 0.399 (−3.35, −11.27 to 4.56) | 0.493 (−2.55, −9.95 to 4.85) |
| OLS Z-statistic, p-value | −1.306, p = 0.192 | 0.656, p = 0.512 |

TABLE 26G
CMAI: Change from Baseline Parallel Group MMRM Analysis - Observed Data
By Age Group (<75, >=75)
mITT 12-Week Parallel Group Population and CMAI Factor 1 Agitated at Baseline
Age <75 Years

| Parameter/Result | Placebo | | AVP-786 18 mg | | AVP-786 28 mg | |
|---|---|---|---|---|---|---|
| Baseline: N, Mean (SD) | 18, 80.1 | (28.49) | 32, 91.8 | (20.05) | 33, 70.6 | (21.92) |
| Week 1 Change from Baseline: N, Mean (SD) | 17, −7.2 | (10.75) | 32, −6.7 | (13.76) | 32, −8.4 | (11.86) |
| Week 1 Change from Baseline: LSMean (SE) | −0.6 | (3.69) | −1.7 | (2.86) | −5.2 | (3.00) |
| TRT Diff vs. PBO: p-value (Diff, 95% CI) | | | 0.738 (−1.16, −8.06 to 5.73) | | 0.186 (−4.63, −11.54 to 2.28) | |
| Week 2 Change from Baseline: N, Mean (SD) | 17, −2.9 | (11.10) | 24, −11.6 | (18.56) | 25, −6.9 | (13.08) |
| Week 2 Change from Baseline: LSMean (SE) | 2.8 | (3.96) | −7.2 | (3.13) | −4.6 | (3.23) |
| TRT Diff vs. PBO: p-value (Diff, 95% CI) | | | 0.013 (−10.03, −17.90 to −2.17 | | 0.064 (−7.42, −15.30 to 0.45) | |
| Week 3 Change from Baseline: N, Mean (SD) | 17, −6.2 | (15.25) | 32, −14.6 | (19.01) | 33, −10.2 | (13.57) |
| Week 3 Change from Baseline: LSMean (SE) | 0.6 | (4.31) | −9.2 | (3.31) | −7.9 | (3.42) |
| TRT Diff vs. PBO: p-value (Diff, 95% CI) | | | 0.030 (−9.78, −18.59 to −0.96 | | 0.063 (−8.37, −17.21 to 0.47) | |
| Week 6 Change from Baseline: N, Mean (SD) | 17, −8.1 | (15.62) | 31, −16.9 | (18.65) | 32, −12.7 | (14.43) |
| Week 6 Change from Baseline: LSMean (SE) | −1.4 | (4.39) | −11.9 | (3.37) | −10.4 | (3.47) |
| TRT Diff vs. PBO: p-value (Diff, 95% CI) | | | 0.023 (−10.55, −19.62 to −1.48 | | 0.052 (−8.99, −18.08 to 0.10) | |
| Week 9 Change from Baseline: N, Mean (SD) | 16, −7.5 | (16.73) | 31, −18.7 | (21.95) | 30, −12.5 | (16.53) |
| Week 9 Change from Baseline: LSMean (SE) | −1.1 | (4.85) | −13.4 | (8.66) | −10.2 | (3.77) |
| TRT Diff vs. PBO: p-value (Diff, 95% CI) | | | 0.020 (−12.30, −22.63 to −1.98 | | 0.085 (−9.11, −19.51 to 1.29) | |
| Week 12 Change from Baseline: N, Mean (SD) | 16, −8.4 | (17.44) | 31, −19.1 | (18.70) | 32, −13.8 | (14.41) |
| Week 12 Change from Baseline: LSMean (SE) | −2.5 | (4.50) | −13.9 | (3.39) | −11.6 | (3.50) |

| | | |
|---|---|---|
| TRT Diff vs. PBO: p-value (Diff, 95% CI) | 0.017 (−11.42, −20.73 to −2.12 | 0.057 (−9.06, −18.42 to 0.29) |

TABLE 26H
CMAI: Change from Baseline Parallel Group MMRM Analysis - Observed Data
By Age Group (<75, >=75)
mITT 12-Week Parallel Group Population and CMAI Factor 1 Agitated at Baseline
Age >=75 Years

| Parameter/Result | Placebo | | AVP-786 18 mg | | AVP-786 28 mg | |
|---|---|---|---|---|---|---|
| Baseline: N, Mean (SD) | 28, 73.4 | (17.97) | 32, 79.3 | (24.34) | 33, 88.2 | (32.52) |
| Week 1 Change from Baseline: N, Mean (SD) | 27, −8.6 | (8.97) | −9.9 | (13.91) | 32, −8.0 | (8.77) |
| Week 1 Change from Baseline: LSMean (SE) | −10.7 | (2.42) | −12.9 | (2.49) | −9.6 | (2.34) |
| TRT Diff vs. PBO: p-value (Diff, 95% CI) | | | 0.435 (−2.25, −7.95 to 3.45) | | 0.706 (1.09, −4.65 to 6.84) | |
| Week 2 Change from Baseline: N, Mean (SD) | 21, −11.9 | (12.33) | 25, −12.0 | (14.40) | 25, −10.5 | (8.61) |
| Week 2 Change from Baseline: LSMean (SE) | −15.1 | (2.50) | −14.6 | (2.54) | −9.8 | (2.42) |
| TRT Diff vs. PBO: p-value (Diff, 95% CI) | | | 0.967 (0.50, −5.43 to 6.44) | | 0.081 (5.34, −0.67 to 11.36 | |
| Week 3 Change from Baseline: N, Mean (SD) | 28, −13.0 | (16.52) | 29, −15.9 | (15.36) | 33, −11.5 | (12.98) |
| Week 3 Change from Baseline: LSMean (SE) | −16.8 | (2.94) | −18.2 | (2.96) | −11.8 | (2.79) |
| TRT Diff vs. PBO: p-value (Diff, 95% CI) | | | 0.719 (−1.32, −9.61 to 5.96) | | 0.156 (5.28, −2.06 to 12.62 | |
| Week 6 Change from Baseline: N, Mean (SD) | 27, −14.9 | (15.39) | 28, −17.5 | (16.54) | 32, −13.6 | (13.07) |
| Week 6 Change from Baseline: LSMean (SE) | −18.5 | (2.93) | −20.4 | (2.96) | −13.9 | (2.78) |
| TRT Diff vs. PBO: p-value (Diff, 95% CI) | | | 0.615 (−1.95, −9.14 to 5.44) | | 0.202 (4.72, −2.58 to 13.02 | |
| Week 9 Change from Baseline: N, Mean (SD) | 26, −14.8 | (16.58) | 27, −18.2 | (16.00) | 32, −13.4 | (14.41) |
| Week 9 Change from Baseline: LSMean (SE) | −19.1 | (2.96) | −21.8 | (3.00) | −13.4 | (2.30) |
| TRT Diff vs. PBO: p-value (Diff, 95% CI) | | | 0.465 (−2.73, −10.13 to 4.67) | | 0.130 (5.69, −1.70 to 13.08 | |
| Week 12 Change from Baseline: N, Mean (SD) | 27, −15.4 | (17.80) | 28, −19.1 | (18.83) | 32, −13.7 | (15.29) |
| Week 12 Change from Baseline: LSMean (SE) | −19.5 | (3.29) | −22.9 | (3.29) | −13.7 | (3.10) |

| | | |
|---|---|---|
| TRT Diff vs. PBO: p-value (Diff, 95% CI) | 0.496 (−3.28, −11.63 to 5.06) | 0.162 (5.93, −2.43 to 14.30 |

TABLE 26I
CMAI Total Score: Change from Baseline SPCD MMRM, Observed Data
By Age Group (<80, >=80)
mITT Population and CMAI Factor 1 Agitated at Baseline
Age <80 Years

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 86, 79.6 | (23.20) | 49, 82.0 | (22.11) | 49, 75.4 | (22.73) |
| Week 1 Change from Baseline: N, Mean (SD) | 83, −6.0 | (9.40) | 49, −8.6 | (14.63) | 48, −9.3 | (10.79) |
| Week 1 Change from Baseline: LSMean (SE) | −5.0 | (1.98) | −7.5 | (2.14) | −9.1 | (2.20) |
| TRT Diff vs. PBO: p-value (Diff, 95% CI) | | | 0.209 (−2.50, −6.40 to 1.40) | | 0.042 (−4.08, −8.00 to −0.15) | |
| Week 2 Change from Baseline: N, Mean (SD) | 70, −7.5 | (11.54) | 37, −14.3 | (18.06) | 36, −9.5 | (11.96) |
| Week 2 Change from Baseline: LSMean (SE) | −6.2 | (2.05) | −12.4 | (2.30) | −9.2 | (2.95) |
| TRT Diff vs. PBO: p-value (Diff, 95% CI) | | | 0.006 (−6.19, −10.53 to −1.90) | | 0.187 (−2.95, −7.36 to 1.45) | |
| Week 3 Change from Baseline: N, Mean (SD) | 84, −10.2 | (12.99) | 49, −16.3 | (18.77) | 49, −11.7 | (13.46) |
| Week 3 Change from Baseline: LSMean (SE) | −9.2 | (2.15) | −15.3 | (2.44) | −12.1 | (2.48) |
| TRT Diff vs. PBO: p-value (Diff, 95% CI) | | | 0.013 (−6.19, −11.04 to −1.34) | | 0.298 (−2.90, −7.74 to 1.94) | |
| Week 6 Change from Baseline: N, Mean (SD) | 83, −12.9 | (14.75) | 47, −19.0 | (18.74) | 47, −14.0 | (14.21) |
| Week 6 Change from Baseline: LSMean (SE) | −11.8 | (2.19) | −17.6 | (2.50) | −14.7 | (2.55) |
| TRT Diff vs. PBO: p-value (Diff, 95% CI) | | | 0.023 (−5.93, −10.86 to −0.80) | | 0.257 (−2.90, −7.93 to 2.13) | |
| Stage 2 (Stage 1 Placebo Non-Responder Only) | | | | | | |
| Baseline: N, Mean (SD) | 20, 72.3 | (23.90) | 21, 71.4 | (21.81) | 20, 61.6 | (16.51) |
| Week 9 Change from Baseline: N, Mean (SD) | 20, 0.6 | (7.01) | 21, −5.2 | (10.27) | 20, −4.1 | (8.04) |
| Week 9 Change from Baseline: LSMean (SE) | 0.8 | (1.92) | −5.1 | (1.96) | −4.5 | (1.94) |
| TRT Diff vs. PBO: p-value (Diff, 95% CI) | | | 0.031 (−5.97, −11.21 to −0.54) | | 0.058 (−5.32, −10.83 to 0.18) | |
| Week 12 Change from Baseline: N, Mean (SD) | 20, −0.6 | (11.70) | 21, −2.8 | (14.09) | 20, −6.4 | (10.98) |
| Week 12 Change from Baseline: LSMean (SE) | −0.2 | (2.77) | −2.6 | (2.69) | −7.0 | (2.79) |

| | | |
|---|---|---|
| TRT Diff vs. PBO: p-value (Diff, 95% CI) | 0.539 (−2.38, −10.07 to 5.32) | 0.091 (−6.83, −14.77 to 1.12) |
| OLS Z-statistic, p-value | −2.052, p = 0.040 | −2.028, p = 0.043 |

TABLE 26J
CMAI Total Score: Change from Baseline SPCD MMRM, Observed Data
By Age Group (<80, >=80)
mITT Population and CMAI Factor 1 Agitated at Baseline
Age >=80 Years

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 57, 77.0 | (21.83) | 15, 73.7 | (23.20) | 17, 90.8 | (23.68) |
| Week 1 Change from Baseline: N, Mean (SD) | 56, −8.7 | (10.83) | 13, −6.8 | (10.53) | 16, −4.8 | (8.25) |
| Week 1 Change from Baseline: LSMean (SE) | −10.0 | (1.95) | −9.3 | (3.22) | −4.4 | (2.84) |
| TRT Diff vs. PBO: p-value (Diff, 95% CI) | | | 0.334 (0.67, −5.64 to 6.97) | | 0.068 (5.63, −0.42 to 11.67) | |
| Week 2 Change from Baseline: N, Mean (SD) | 44, −10.9 | (11.52) | 12, −4.2 | (4.61) | 14, −6.6 | (8.56) |
| Week 2 Change from Baseline: LSMean (SE) | −19.0 | (1.96) | −7.2 | (3.10) | −5.5 | (2.78) |
| TRT Diff vs. PBO: p-value (Diff, 95% CI) | | | 0.061 (5.80, −0.27 to 11.38) | | 0.013 (7.52, 1.61 to 13.43) | |
| Week 3 Change from Baseline: N, Mean (SD) | 56, −13.5 | (15.51) | 13, −9.5 | (9.91) | 17, −8.5 | (12.37) |
| Week 3 Change from Baseline: LSMean (SE) | −15.2 | (2.24) | −11.2 | (3.85) | −6.6 | (3.44) |
| TRT Diff vs. PBO: p-value (Diff, 95% CI) | | | 0.318 (3.97, −3.89 to 11.83) | | 0.026 (8.56, 1.05 to 16.08) | |
| Week 6 Change from Baseline: N, Mean (SD) | 54, −15.0 | (15.56) | 12, −10.2 | (9.15) | 17, −10.8 | (12.12) |
| Week 6 Change from Baseline: LSMean (SE) | −16.4 | (2.26) | −13.2 | (4.04) | −8.9 | (3.45) |
| TRT Diff vs. PBO: p-value (Diff, 95% CI) | | | 0.445 (3.19, −5.08 to 11.45) | | 0.052 (7.49, −0.08 to 15.07) | |
| Stage 2 (Stage 1 Placebo Non-Responder Only) | | | | | | |
| Baseline: N, Mean (SD) | 9, 63.7 | (20.32) | 9, 67.3 | (23.89) | 15, 74.1 | (22.72) |
| Week 9 Change from Baseline: N, Mean (SD) | −0.3 | (8.46) | 9, −7.8 | (10.38) | 15, −1.7 | (13.82) |
| Week 9 Change from Baseline: LSMean (SE) | −2.2 | (2.93) | −8.4 | (2.90) | 0.1 | (2.28) |
| TRT Diff vs. PBO: p-value (Diff, 95% CI) | | | 0.146 (−6.14, −14.55 to 2.27) | | 0.539 (2.93, −5.33 to 9.93) | |
| Week 12 Change from Baseline: N, Mean (SD) | 9, −2.4 | (7.18) | 9, −11.8 | (10.06) | 14, −4.1 | (15.15) |
| Week 12 Change from Baseline: LSMean (SE) | −4.4 | (2.99) | −12.4 | (2.97) | −2.6 | (2.37) |

| | | |
|---|---|---|
| TRT Diff vs. PBO: p-value (Diff, 95% CI) | 0.067 (−8.01, −16.81 to 0.59) | 0.647 (1.78, −6.09 to 9.65) |
| OLS Z-statistic, p-value | −0.429, p = 0.668 | 1.892, p = 0.058 |

TABLE 26K
CMAI: Change from Baseline Parallel Group MMRM Analysis - Observed Data
By Age Group (<80, >=80)
mITT 12-Week Parallel Group Population and CMAI Factor 1 Agitated at Baseline
Age <80 Years

| Parameter/Result | Placebo | | AVP-786 18 mg | | AVP-786 28 mg | |
|---|---|---|---|---|---|---|
| Baseline: N, Mean (SD) | 27, 77.5 | (24.66) | 49, 32.0 | (22.11) | 49, 75.4 | (23.73) |
| Week 1 Change from Baseline: N, Mean (SD) | 26, −5.8 | (9.30) | 49, −8.6 | (14.63) | 48, −9.3 | (10.79) |
| Week 1 Change from Baseline: LSMean (SE) | −4.3 | (3.13) | −7.1 | (2.48) | −9.9 | (2.55) |
| TRT Diff vs. PBO: p-value (Diff, 95% CI) | | | 0.323 (−2.84, −8.50 to 2.83) | | 0.106 (−4.64, −10.29 to 1.00) | |
| Week 2 Change from Baseline: N, Mean (SD) | 23, −3.5 | (9.85) | 37, −14.9 | (18.06) | 36, −9.5 | (11.96) |
| Week 2 Change from Baseline: LSMean (SE) | −2.1 | (3.26) | −12.1 | (2.60) | −9.9 | (2.66) |
| TRT Diff vs. PBO: p-value (Diff, 95% CI) | | | 0.002 (−10.07, −16.22 to −3.93 | | 0.028 (−6.87, −12.99 to −0.75 | |
| Week 3 Change from Baseline: N, Mean (SD) | 26, −5.6 | (12.39) | 48, −16.9 | (18.77) | 49, −11.7 | (13.46) |
| Week 3 Change from Baseline: LSMean (SE) | −4.3 | (8.47) | −14.8 | (2.72) | −11.8 | (2.77) |
| TRT Diff vs. PBO: p-value (Diff, 95% CI) | | | 0.003 (−10.49, −17.25 to −3.72 | | 0.029 (−7.31, −14.23 to −0.80 | |
| Week 6 Change from Baseline: N, Mean (SD) | 26, −8.2 | (14.16) | 47, −19.0 | (18.74) | 47, −14.0 | (14.21) |
| Week 6 Change from Baseline: LSMean (SE) | −7.0 | (3.57) | −17.3 | (2.80) | −14.3 | (2.85) |
| TRT Diff vs. PBO: p-value (Diff, 95% CI) | | | 0.005 (−10.35, −17.44 to −3.25 | | 0.041 (−7.35, −14.40 to −0.30 | |
| Week 9 Change from Baseline: N, Mean (SD) | 25, −7.4 | (14.85) | 47, −20.9 | (20.26) | 46, −14.6 | (15.75) |
| Week 9 Change from Baseline: LSMean (SE) | −6.5 | (3.75) | −19.0 | (2.91) | −14.7 | (2.98) |
| TRT Diff vs. PBO: p-value (Diff, 95% CI) | | | 0.001 (−12.49, −20.10 to −4.88 | | 0.033 (−9.24, −15.90 to −0.67 | |
| Week 12 Change from Baseline: N, Mean (SD) | 25, −8.2 | (15.49) | 47, −21.9 | (19.59) | 48, −15.0 | (14.92) |
| Week 12 Change from Baseline: LSMean (SE) | −7.4 | (3.72) | −19.9 | (2.68) | −15.3 | (2.92) |

| | | |
|---|---|---|
| TRT Diff vs. PBO: p-value (Diff, 95% CI) | 0.001 (−12.51, −20.02 to −5.00 | 0.039 (−7.85, −15.30 to −0.41 |

TABLE 26L
CMAI: Change from Baseline Parallel Group MMRM Analysis - Observed Data
By Age Group (<80, >=80)
mITT 12-Week Parallel Group Population and CMAI Factor 1 Agitated at Baseline
Age >=80 Years

| Parameter/Result | Placebo | | AVP-786 18 mg | | AVP-786 28 mg | |
|---|---|---|---|---|---|---|
| Baseline: N, Mean (SD) | 19, 73.8 | (19.79) | 15, 73.7 | (13.20) | 17, 90.8 | (13.68) |
| Week 1 Change from Baseline: N, Mean (SD) | 18, −11.2 | (9.24) | 13, −6.8 | (10.53) | 16, −4.8 | (8.25) |
| Week 1 Change from Baseline: LSMean (SE) | −11.0 | (2.51) | −7.9 | (3.13) | −4.6 | (2.76) |
| TRT Diff vs. PBO: p-value (Diff, 95% CI) | | | 0.389 (3.11, −4.08 to 10.30 | | 0.074 (6.47, −0.64 to 13.50 | |
| Week 2 Change from Baseline: N, Mean (SD) | 15, −14.5 | (13.43) | 12, −4.2 | (4.61) | 14, −6.6 | (8.56) |
| Week 2 Change from Baseline: LSMean (SE) | −16.2 | (2.53) | −5.7 | (3.06) | −4.8 | (2.74) |
| TRT Diff vs. PBO: p-value (Diff, 95% CI) | | | 0.005 (10.46, 3.39 to 17.53 | | 0.002 (11.42, 4.43 to 18.41 | |
| Week 3 Change from Baseline: N, Mean (SD) | 19, −17.1 | (18.20) | 13, −9.5 | (9.91) | 17, −8.5 | (12.37) |
| Week 3 Change from Baseline: LSMean (SE) | −18.3 | (3.37) | −9.6 | (4.05) | −6.2 | (3.58) |
| TRT Diff vs. PBO: p-value (Diff, 95% CI) | | | 0.085 (8.63, −1.23 to 18.49 | | 0.017 (12.06, 2.27 to 21.85 | |
| Week 6 Change from Baseline: N, Mean (SD) | 18, −18.1 | (16.25) | 12, −10.2 | (9.15) | 17, −10.8 | (12.12) |
| Week 6 Change from Baseline: LSMean (SE) | −18.8 | (3.14) | −12.0 | (3.85) | −9.4 | (3.37) |
| TRT Diff vs. PBO: p-value (Diff, 95% CI) | | | 0.144 (6.83, −2.41 to 16.08 | | 0.024 (10.41, 1.43 to 19.39 | |
| Week 9 Change from Baseline: N, Mean (SD) | 17, −18.9 | (17.61) | 11, −8.0 | (8.66) | 16, −8.2 | (13.49) |
| Week 9 Change from Baseline: LSMean (SE) | −20.1 | (3.30) | −12.7 | (4.10) | −5.8 | (3.55) |
| TRT Diff vs. PBO: p-value (Diff, 95% CI) | | | 0.132 (7.48, −2.34 to 17.29 | | 0.004 (14.37, 4.89 to 23.85 | |
| Week 12 Change from Baseline: N, Mean (SD) | 18, −19.3 | (19.14) | 12, −8.3 | (8.03) | 16, −10.1 | (14.00) |
| Week 12 Change from Baseline: LSMean (SE) | −20.4 | (3.61) | −12.7 | (4.43) | −8.2 | (3.90) |

| | | |
|---|---|---|
| TRT Diff vs. PBO: p-value (Diff, 95% CI) | 0.156 (7.71, −3.05 to 18.46 | 0.024 (12.19, 1.70 to 12.68 |

TABLE 26M
CMAI Total Score: Change from Baseline SPCD MMRM, Observed Data
By Baseline Antipsychotics Use
mITT Population and CMAI Factor 1 Agitated at Baseline
Baseline Antipsychotic: Yes

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 55, 82.7 | (25.01) | 23, 86.3 | (20.28) | 25, 94.7 | (23.31) |
| Week 1 Change from Baseline: N, Mean (SD) | 54, −7.6 | (11.03) | 22, −10.5 | (18.25) | 23, −7.5 | (11.34) |
| Week 1 Change from Baseline: LSMean (SE) | −10.2 | (2.64) | −12.5 | (3.36) | −9.2 | (3.19) |
| TRT Diff vs. PBO: p-value (Diff, 95% CI) | | | 0.455 (−2.29, −8.34 to 3.77) | | 0.749 (0.99, −4.91 to 6.39) | |
| Week 2 Change from Baseline: N, Mean (SD) | 41, −9.9 | (14.02) | 20, −14.8 | (21.93) | 19, −8.1 | (11.01) |
| Week 2 Change from Baseline: LSMean (SE) | −12.8 | (2.81) | −17.4 | (3.62) | −11.0 | (3.46) |
| TRT Diff vs. PBO: p-value (Diff, 95% CI) | | | 0.189 (−4.59, −11.47 to 2.30) | | 0.605 (1.76, −4.98 to 3.50) | |
| Week 3 Change from Baseline: N, Mean (SD) | 54, −12.8 | (15.89) | 23, −22.2 | (22.80) | 25, −8.3 | (12.16) |
| Week 3 Change from Baseline: LSMean (SE) | −15.5 | (2.94) | −22.7 | (9.88) | −10.3 | (3.82) |
| TRT Diff vs. PBO: p-value (Diff, 95% CI) | | | 0.064 (−7.18, −14.79 to 0.43) | | 0.166 (5.19, −2.19 to 12.57) | |
| Week 6 Change from Baseline: N, Mean (SD) | 53, −13.1 | (16.59) | 22, −23.0 | (23.21) | 25, −12.5 | (14.94) |
| Week 6 Change from Baseline: LSMean (SE) | −15.8 | (3.02) | −25.0 | (4.04) | −14.5 | (3.82) |
| TRT Diff vs. PBO: p-value (Diff, 95% CI) | | | 0.025 (−9.27, −17.34 to −1.21) | | 0.750 (1.25, −6.52 to 9.02) | |
| Stage 2 (Stage 1 Placebo Non-Responder Only) | | | | | | |
| Baseline: N, Mean (SD) | 12, 78.2 | (34.21) | 10, 80.5 | (22.91) | 15, 67.1 | (21.15) |
| Week 9 Change from Baseline: N, Mean (SD) | 12, 0.2 | (8.77) | 10, −7.6 | (12.60) | 15, −5.1 | (13.24) |
| Week 9 Change from Baseline: LSMean (SE) | 1.0 | (3.08) | −6.3 | (3.39) | −6.5 | (2.79) |
| TRT Diff vs. PBO: p-value (Diff, 95% CI) | | | 0.119 (−7.29, −16.55 to 1.97) | | 0.082 (−7.50, −16.01 to 1.01) | |
| Week 12 Change from Baseline: N, Mean (SD) | 12, 1.1 | (11.15) | 10, −9.3 | (9.64) | 15, −6.7 | (15.09) |
| Week 12 Change from Baseline: LSMean (SE) | 1.6 | (3.53) | −3.4 | (3.86) | −7.7 | (3.19) |

| | | |
|---|---|---|
| TRT Diff vs. PBO: p-value (Diff, 95% CI) | 0.063 (−10.05, −20.66 to 0.57) | 0.060 (−9.35, −19.10 to 0.41) |
| OLS Z-statistic, p-value | −2.986, p = 0.003 | −0.986, p = 0.324 |

TABLE 26N
CMAI Total Score: Change from Baseline SPCD MMRM, Observed Data
By Baseline Antipsychotics Use
mITT Population and CMAI Factor 1 Agitated at Baseline
Baseline Antipsychotic: No

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 88, 75.9 | (19.92) | 41, 76.6 | (23.11) | 41, 76.2 | (23.77) |
| Week 1 Change from Baseline: N, Mean (SD) | 85, −6.8 | (8.43) | 40, −7.0 | (10.72) | 41, −8.6 | (9.55) |
| Week 1 Change from Baseline: LSMean (SE) | −5.9 | (1.59) | −6.4 | (1.88) | −7.9 | (1.86) |
| TRT Diff vs. PBO: p-value (Diff, 95% CI) | | | 0.780 (−0.52, −4.18 to 3.14) | | 0.262 (−2.09, −5.72 to 1.57) | |
| Week 2 Change from Baseline: N, Mean (SD) | 73, −8.2 | (10.05) | 29, −9.7 | (11.11) | 31, −9.0 | (11.34) |
| Week 2 Change from Baseline: LSMean (SE) | −7.5 | (1.62) | −9.0 | (2.00) | −7.6 | (1.97) |
| TRT Diff vs. PBO: p-value (Diff, 95% CI) | | | 0.458 (−1.50, −5.47 to 2.48) | | 0.980 (−0.10, −4.03 to 3.33) | |
| Week 3 Change from Baseline: N, Mean (SD) | 86, −10.7 | (12.87) | 38, −11.6 | (12.19) | 41, −12.5 | (13.65) |
| Week 3 Change from Baseline: LSMean (SE) | −9.9 | (1.76) | −10.6 | (2.22) | −11.9 | (2.18) |
| TRT Diff vs. PBO: p-value (Diff, 95% CI) | | | 0.776 (−0.67, −5.28 to 3.95) | | 0.330 (−2.08, −6.58 to 2.52) | |
| Week 6 Change from Baseline: N, Mean (SD) | 84, −14.1 | (14.10) | 37, −13.8 | (12.16) | 39, −13.6 | (12.96) |
| Week 6 Change from Baseline: LSMean (SE) | −13.2 | (1.77) | −13.0 | (2.25) | −13.2 | (2.21) |
| TRT Diff vs. PBO: p-value (Diff, 95% CI) | | | 0.924 (0.23, −4.47 to 4.92) | | 0.984 (−0.05, −4.67 to 4.57) | |
| Stage 2 (Stage 1 Placebo Non-Responder Only) | | | | | | |
| Baseline: N, Mean (SD) | 17, 64.1 | (18.42) | 20, 65.1 | (20.36) | 20, 66.9 | (19.85) |
| Week 9 Change from Baseline: N, Mean (SD) | 17, 0.4 | (8.44) | 20, −5.2 | (9.01) | 20, −1.6 | (8.55) |
| Week 9 Change from Baseline: LSMean (SE) | 0.3 | (1.95) | −5.2 | (1.80) | −1.4 | (1.80) |
| TRT Diff vs. PBO: p-value (Diff, 95% CI) | | | 0.044 (−5.47, −10.80 to −0.14) | | 0.535 (−1.66, −7.00 to 3.67) | |
| Week 12 Change from Baseline: N, Mean (SD) | 17, −2.7 | (9.89) | 20, −3.6 | (14.93) | 19, −4.5 | (10.77) |
| Week 12 Change from Baseline: LSMean (SE) | −2.9 | (2.84) | −3.6 | (2.62) | −4.5 | (2.68) |

| | | |
|---|---|---|
| TRT Diff vs. PBO: p-value (Diff, 95% CI) | 0.854 (−0.72, −8.47 to 7.04) | 0.685 (−1.60, −9.43 to 6.24) |
| OLS Z-statistic, p-value | −0.072, p = 0.943 | −0.313, p = 0.751 |

TABLE 26O
Appendix 621.4B
CMAI: Change from Baseline Parallel Group MMRM Analysis - Observed Data
By Baseline Antipsychotics Use
mITT 12-Week Parallel Group Population and CMAI Factor 1 Agitated at Baseline
Baseline Antipsychotic: Yes

| Parameter/Result | Placebo | | AVP-786 18 mg | | AVP-786 28 mg | |
|---|---|---|---|---|---|---|
| Baseline: N, Mean (SD) | 18, 85.4 | (30.28) | 23, 86.3 | (20.28) | 25, 84.7 | (23.31) |
| Week 1 Change from Baseline: N, Mean (SD) | 18, −8.6 | (9.48) | 22, −10.5 | (18.25) | 23, −7.5 | (11.84) |
| Week 1 Change from Baseline: LSMean (SE) | −9.5 | (3.49) | −11.6 | (3.75) | −8.5 | (3.56) |
| TRT Diff vs. PBO: p-value (Diff, 95% CI) | | | 0.626 (−2.10, −10.66 to 6.47) | | 0.806 (1.03, −7.32 to 9.37) | |
| Week 2 Change from Baseline: N, Mean (SD) | 14, −11.5 | (14.81) | 20, −14.8 | (21.93) | 19, −9.1 | (11.01) |
| Week 2 Change from Baseline: LSMean (SE) | −11.4 | (3.86) | −16.8 | (3.97) | −10.3 | (3.80) |
| TRT Diff vs. PBO: p-value (Diff, 95% CI) | | | 0.264 (−5.38, −14.92 to 4.16) | | 0.804 (1.16, −8.19 to 10.51 | |
| Week 3 Change from Baseline: N, Mean (SD) | 18, −13.4 | (17.08) | 23, −21.2 | (23.80) | 25, −8.3 | (12.16) |
| Week 3 Change from Baseline: LSMean (SE) | −14.4 | (4.24) | −22.0 | (4.29) | −9.6 | (4.08) |
| TRT Diff vs. PBO: p-value (Diff, 95% CI) | | | 0.163 (−7.53, −18.21 to 3.14) | | 0.360 (4.80, −5.62 to 15.22 | |
| Week 6 Change from Baseline: N, Mean (SD) | 18, −14.1 | (15.29) | 22, −23.0 | (23.21) | 25, −12.5 | (14.94) |
| Week 6 Change from Baseline: LSMean (SE) | −15.0 | (4.30) | −24.3 | (4.36) | −13.8 | (4.13) |
| TRT Diff vs. PBO: p-value (Diff, 95% CI) | | | 0.093 (−9.29, −20.17 to 1.60) | | 0.821 (1.21, −9.40 to 11.91 | |
| Week 9 Change from Baseline: N, Mean (SD) | 17, −14.5 | (17.91) | 21, −23.0 | (26.61) | 24, −10.9 | (16.18) |
| Week 9 Change from Baseline: LSMean (SE) | −15.5 | (4.81) | −23.7 | (4.77) | −13.2 | (4.51) |
| TRT Diff vs. PBO: p-value (Diff, 95% CI) | | | 0.190 (−8.16, −20.49 to 4.16) | | 0.706 (2.28, −9.73 to 14.29 | |
| Week 12 Change from Baseline: N, Mean (SD) | 17, −14.3 | (21.60) | 22, −25.4 | (23.70) | 24, −11.8 | (17.84) |
| Week 12 Change from Baseline: LSMean (SE) | −15.3 | (4.96) | −28.6 | (4.86) | −13.7 | (4.62) |

| | | |
|---|---|---|
| TRT Diff vs. PBO: p-value (Diff, 95% CI) | 0.080 (−11.33, −24.04 to 1.39) | 0.802 (1.57, −10.86 to 13.99 |

TABLE 26P
CMAI: Change from Baseline Parallel Group MMRM Analysis - Observed Data
By Baseline Antipsychotics Use
mITT 12-Week Parallel Group Population and CMAI Factor 1 Agitated at Baseline
Baseline Antipsychotic: No

| Parameter/Result | Placebo | | AVP-786 18 mg | | AVP-786 28 mg | |
|---|---|---|---|---|---|---|
| Baseline: N, Mean (SD) | 28, 69.9 | (13.30) | 41, 76.6 | (23.11) | 41, 76.2 | (23.77) |
| Week 1 Change from Baseline: N, Mean (SD) | 26, −7.7 | (9.75) | 40, −7.0 | (10.72) | 41, −8.6 | (9.55) |
| Week 1 Change from Baseline: LSMean (SE) | −7.5 | (2.58) | −7.0 | (2.10) | −8.6 | (2.08) |
| TRT Diff vs. PBO: p-value (Diff, 95% CI) | | | 0.334 (0.53, −4.51 to 5.58) | | 0.683 (−1.04, −6.07 to 3.99) | |
| Week 2 Change from Baseline: N, Mean (SD) | 24, −5.7 | (10.67) | 29, −9.7 | (11.11) | 31, −9.0 | (11.34) |
| Week 2 Change from Baseline: LSMean (SE) | −7.6 | (2.67) | −9.6 | (2.25) | −8.1 | (2.21) |
| TRT Diff vs. PBO: p-value (Diff, 95% CI) | | | 0.464 (−2.03, −7.49 to 3.44) | | 0.853 (−0.51, −5.93 to 4.91) | |
| Week 3 Change from Baseline: N, Mean (SD) | 27, −8.4 | (15.62) | 33, −11.6 | (12.19) | 41, −12.5 | (13.65) |
| Week 3 Change from Baseline: LSMean (SE) | −9.2 | (2.95) | −10.8 | (2.44) | −12.3 | (2.40) |
| TRT Diff vs. PBO: p-value (Diff, 95% CI) | | | 0.625 (−1.55, −7. 84 to 4.74) | | 0.932 (−3.07, −9.31 to 3.17) | |
| Week 6 Change from Baseline: N, Mean (SD) | 26, −11.0 | (16.08) | 37, −13.8 | (12.16) | 39, −13.6 | (12.96) |
| Week 6 Change from Baseline: LSMean (SE) | −11.8 | (2.98) | −13.2 | (3.46) | −13.8 | (2.41) |
| TRT Diff vs. PBO: p-value (Diff, 95% CI) | | | 0.657 (−1.44, −7.83 to 4.95) | | 0.524 (−2.04, −8.36 to 4.29) | |
| Week 9 Change from Baseline: N, Mean (SD) | 25, −10.4 | (16.20) | 37, −15.9 | (13.20) | 39, −14.3 | (14.89) |
| Week 9 Change from Baseline: LSMean (SE) | −11.9 | (3.03) | −16.0 | (2.53) | −13.9 | (2.49) |
| TRT Diff vs. PBO: p-value (Diff, 95% CI) | | | 0.209 (−4.08, −10.73 to 2.60) | | 0.558 (−1.96, −8.57 to 4.88) | |
| Week 12 Change from Baseline: N, Mean (SD) | 26, −11.8 | (15.18) | 37, −15.4 | (13.89) | 40, −15.0 | (12.60) |
| Week 12 Change from Baseline: LSMean (SE) | −13.1 | (2.97) | −15.4 | (2.44) | −15.0 | (2.38) |

| | | |
|---|---|---|
| TRT Diff vs. PBO: p-value (Diff, 95% CI) | 0.493 (−2.25, −8.58 to 4.09) | 0.550 (−1.89, −8.13 to 4.35) |

TABLE 26Q
CMAI Total Score: Change from Baseline SPCD MMRM, Observed Data
By Anti-Dementia Med Use
mITT Population and CMAI Factor 1 Agitated at Baseline
On Treatment Anti-Dementia Med Use: Yes

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 117, 79.3 | (29.45) | 56, 81.0 | (23.69) | 54, 79.8 | (24.77) |
| Week 1 Change from Baseline: N, Mean (SD) | 113, −6.7 | (8.85) | 54, −8.4 | (14.25) | 52, −7.9 | (10.33) |
| Week 1 Change from Baseline: LSMean (SE) | −6.5 | (1.53) | −8.4 | (1.80) | −7.7 | (1.84) |
| TRT Diff vs. PBO: p-value (Diff, 95% CI) | | | 0.283 (−1.86, −5.27 to 1.54) | | 0.507 (−1.16, −4.60 to 2.28) | |
| Week 2 Change from Baseline: N, Mean (SD) | 96, −9.3 | (11.76) | 43, −12.8 | (17.11) | 40, −8.0 | (11.07) |
| Week 2 Change from Baseline: LSMean (SE) | −3.8 | (1.62) | −11.9 | (1.98) | −8.1 | (2.02) |
| TRT Diff vs. PBO: p-value (Diff, 95% CI) | | | 0.120 (−3.09, −7.00 to 0.81) | | 0.720 (0.72, −3.23 to 4.67) | |
| Week 3 Change from Baseline: N, Mean (SD) | 114, −11.3 | (14.06) | 54, −16.1 | (17.89) | 54, −10.9 | (13.16) |
| Week 3 Change from Baseline: LSMean (SE) | −11.3 | (1.73) | −15.7 | (2.16) | −10.9 | (2.19) |
| TRT Diff vs. PBO: p-value (Diff, 95% CI) | | | 0.050 (−4.43, −8.85 to 0.00) | | 0.869 (0.37, −4.07 to 4.81) | |
| Week 6 Change from Baseline: N, Mean (SD) | 111, −14.0 | (14.76) | 53, −18.5 | (17.82) | 52, −12.8 | (13.27) |
| Week 6 Change from Baseline: LSMean (SE) | −13.8 | (1.74) | −18.3 | (2.17) | −12.9 | (2.20) |
| TRT Diff vs. PBO: p-value (Diff, 95% CI) | | | 0.051 (−4.45, −8.92 to 0.03) | | 0.688 (0.92, −3.58 to 5.41) | |
| Stage 2 (Stage 1 Placebo Non-Responder Only) | | | | | | |
| Baseline: N, Mean (SD) | 22, 72.7 | (28.58) | 24, 69.0 | (20.92) | 29, 86.6 | (21.80) |
| Week 9 Change from Baseline: N, Mean (SD) | 22, 0.0 | (3.02) | 24, −7.5 | (10.07) | 29, −3.3 | (11.31) |
| Week 9 Change from Baseline: LSMean (SE) | 0.7 | (1.97) | −7.5 | (1.88) | −4.2 | (1.71) |
| TRT Diff vs. PBO: p-value (Diff, 95% CI) | | | 0.004 (−8.15, −13.58 to −2.72) | | 0.070 (−4.83, −10.05 to 0.40) | |
| Week 12 Change from Baseline: N, Mean (SD) | 22, −1.1 | (10.46) | 24, −5.5 | (14.67) | 28, −6.8 | (13.14) |
| Week 12 Change from Baseline: LSMean (SE) | −0.5 | (2.65) | −5.5 | (2.52) | −7.2 | (2.33) |

| | | |
|---|---|---|
| TRT Diff vs. PBO: p-value (Diff, 95% CI) | 0.173 (−5.04, −12.33 to 2.26) | 0.062 (−6.72, −13.78 to 0.34) |
| OLS Z-statistic, p-value | −2.343, p = 0.019 | −1.086, p = 0.277 |

TABLE 26R
CMAI Total Score: Change from Baseline SPCD MMRM, Observed Data
By Anti-Dementia Med Use
mITT Population and CMAI Factor 1 Agitated at Baseline
On Treatment Anti-Dementia Med Use: No

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 26, 75.2 | (18.47) | 8, 73.5 | (8.62) | 12, 77.6 | (19.49) |
| Week 1 Change from Baseline: N, Mean (SD) | 26, −8.7 | (14.27) | 8, −6.9 | (11.05) | 12, −9.5 | (10.78) |
| Week 1 Change from Baseline: LSMean (SE) | −8.9 | (3.84) | −8.5 | (5.78) | −9.6 | (4.32) |
| TRT Diff vs. PBO: p-value (Diff, 95% CI) | | | 0.941 (0.40, −10.32 to 11.12) | | 0.392 (−0.62, −9.74 to 8.51) | |
| Week 2 Change from Baseline: N, Mean (SD) | 18, −6.4 | (10.74) | 6, −4.3 | (6.77) | 10, −11.4 | (11.41) |
| Week 2 Change from Baseline: LSMean (SE) | −8.8 | (3.78) | −9.0 | (5.83) | −10.0 | (4.16) |
| TRT Diff vs. PBO: p-value (Diff, 95% CI) | | | 0.962 (−0.24, −10.61 to 10.13) | | 0.773 (−1.26, −10.09 to 7.57) | |
| Week 3 Change from Baseline: N, Mean (SD) | 26, −12.2 | (14.52) | 7, −8.1 | (12.38) | 12, −10.7 | (13.77) |
| Week 3 Change from Baseline: LSMean (SE) | −12.4 | (3.99) | −8.5 | (6.17) | −10.8 | (4.60) |
| TRT Diff vs. PBO: p-value (Diff, 95% CI) | | | 0.510 (3.87, −7.89 to 15.63) | | 0.740 (1.64, −8.25 to 11.52) | |
| Week 6 Change from Baseline: N, Mean (SD) | 26, −12.3 | (16.51) | 8, −5.7 | (9.33) | 12, −14.8 | (15.77) |
| Week 6 Change from Baseline: LSMean (SE) | −12.5 | (4.17) | −8.3 | (6.74) | −14.8 | (4.94) |
| TRT Diff vs. PBO: p-value (Diff, 95% CI) | | | 0.521 (4.23, −8.96 to 17.43) | | 0.677 (−2.25, −13.10 to 8.61) | |
| Stage 2 (Stage 1 Placebo Non-Responder Only) | | | | | | |
| Baseline: N, Mean (SD) | 7, 61.3 | (17.55) | 6, 75.2 | (28.07) | 6, 68.8 | (11.55) |
| Week 9 Change from Baseline: N, Mean (SD) | 7, 1.1 | (5.05) | 6, 0.0 | (9.06) | 6, 0.3 | (7.58) |
| Week 9 Change from Baseline: LSMean (SE) | 1.0 | (2.91) | 0.2 | (3.14) | 0.4 | (3.07) |
| TRT Diff vs. PBO: p-value (Diff, 95% CI) | | | 0.852 (−0.83, −10.16 to 8.50) | | 0.882 (−0.64, −9.68 to 8.40) | |
| Week 12 Change from Baseline: N, Mean (SD) | 7, −1.1 | (11.07) | 6, −5.3 | (8.21) | 6, 0.7 | (8.69) |
| Week 12 Change from Baseline: LSMean (SE) | −2.0 | (3.67) | −4.4 | (3.95) | 0.8 | (3.87) |

| | | |
|---|---|---|
| TRT Diff vs. PBO: p-value (Diff, 95% CI) | 0.668 (−2.41, −14.17 to 9.34) | 0.611 (2.78, −8.61 to 14.16) |
| OLS Z-statistic, p-value | 0.350, p = 0.727 | −0.062, p = 0.951 |

TABLE 26S
CMAI: Change from Baseline Parallel Group MMRM Analysis - Observed Data
By Anti-Dementia Med Use
mITT 12-Week Parallel Group Population and CMAI Factor 1 Agitated at Baseline
On Treatment Anti-Dementia Med Use: Yes

| Parameter/Result | Placebo | | AVP-786 18 mg | | AVP-786 28 mg | |
|---|---|---|---|---|---|---|
| Baseline: N, Mean (SD) | 36, 77.4 | (24.87) | 56, 81.0 | (23.69) | 54, 79.9 | (24.77) |
| Week 1 Change from Baseline: N, Mean (SD) | 34, −6.7 | (7.88) | 54, −8.4 | (14.25) | 52, −7.9 | (10.39) |
| Week 1 Change from Baseline: LSMean (SE) | −5.9 | (2.33) | −7.9 | (2.03) | −7.3 | (2.07) |
| TRT Diff vs. PBO: p-value (Diff, 95% CI) | | | 0.420 (−1.99, −6.36 to 2.88) | | 0.581 (−1.37, −6.26 to 3.53) | |
| Week 2 Change from Baseline: N, Mean (SD) | 31, −8.6 | (12.20) | 43, −12.8 | (17.11) | 40, −8.0 | (11.07) |
| Week 2 Change from Baseline: LSMean (SE) | −9.5 | (2.52) | −11.6 | (2.20) | −7.8 | (2.24) |
| TRT Diff vs. PBO: p-value (Diff, 95% CI) | | | 0.265 (−3.09, −8.57 to 2.38) | | 0.784 (0.77, −4.74 to 6.27) | |
| Week 3 Change from Baseline: N, Mean (SD) | 35, −10.2 | (16.80) | 54, −16.1 | (17.89) | 54, −10.9 | (13.16) |
| Week 3 Change from Baseline: LSMean (SE) | −9.9 | (2.80) | −15.1 | (2.39) | −10.5 | (2.43) |
| TRT Diff vs. PBO: p-value (Diff, 95% CI) | | | 0.096 (−5.32, −11.60 to 0.96) | | 0.838 (−0.65, −6.94 to 5.64) | |
| Week 6 Change from Baseline: N, Mean (SD) | 34, −11.9 | (15.47) | 53, −18.5 | (17.82) | 52, −12.8 | (13.27) |
| Week 6 Change from Baseline: LSMean (SE) | −11.5 | (2.77) | −17.8 | (2.36) | −12.6 | (2.40) |
| TRT Diff vs. PBO: p-value (Diff, 95% CI) | | | 0.045 (−6.32, −12.49 to −0.14 | | 0.715 (−1.14, −7.33 to 5.04) | |
| Week 9 Change from Baseline: N, Mean (SD) | 32, −12.1 | (17.75) | 52, −18.8 | (19.72) | 50, −12.4 | (15.78) |
| Week 9 Change from Baseline: LSMean (SE) | −12.2 | (3.04) | −18.8 | (2.54) | −12.3 | (2.59) |
| TRT Diff vs. PBO: p-value (Diff, 95% CI) | | | 0.092 (−6.19, −13.04 to 0.78) | | 0.979 (−0.09, −7.02 to 6.84) | |
| Week 12 Change from Baseline: N, Mean (SD) | 33, −12.7 | (17.80) | 53, −20.2 | (19.03) | 52, −13.2 | (14.58) |
| Week 12 Change from Baseline: LSMean (SE) | −12.5 | (2.96) | −19.3 | (2.49) | −13.2 | (2.53) |

| | | |
|---|---|---|
| TRT Diff vs. PBO: p-value (Diff, 95% CI) | 0.034 (−7.28, −14.00 to −0.56 | 0.824 (−0.76, −7.49 to 5.97) |

TABLE 26T
CMAI: Change from Baseline Parallel Group MMRM Analysis - Observed Data
By Anti-Dementia Med Use
mITT 12-Week Parallel Group Population and CMAI Factor 1 Agitated at Baseline
On Treatment Anti-Dementia Med Use: No

| Parameter/Result | Placebo | | AVP-786 18 mg | | AVP-786 28 mg | |
|---|---|---|---|---|---|---|
| Baseline: N, Mean (SD) | 10, 70.9 | (10.68) | 8, 73.5 | (8.62) | 12, 77.6 | (19.49) |
| Week 1 Change from Baseline: N, Mean (SD) | 10, −12.4 | (13.42) | 8, −8.9 | (11.05) | 12, −9.5 | (10.78) |
| Week 1 Change from Baseline: LSMean (SE) | −13.9 | (4.97) | −7.7 | (6.11) | −9.4 | (4.55) |
| TRT Diff vs. PBO: p-value (Diff, 95% CI) | | | 0.328 (6.20, −6.61 to 19.01 | | 0.415 (4.48, −8.87 to 15.62 | |
| Week 2 Change from Baseline: N, Mean (SD) | 7, −4.6 | (14.22) | 6, −4.3 | (6.77) | 10, −11.4 | (11.41) |
| Week 2 Change from Baseline: LSMean (SE) | −7.8 | (5.23) | −7.5 | (6.41) | −9.7 | (4.75) |
| TRT Diff vs. PBO: p-value (Diff, 95% CI) | | | 0.964 (0.30, −13.36 to 13.96 | | 0.756 (−1.82, −13.86 to 10.21 | |
| Week 3 Change from Baseline: N, Mean (SD) | 10, −11.2 | (14.82) | 7, −8.1 | (12.38) | 12, −10.7 | (13.77) |
| Week 3 Change from Baseline: LSMean (SE) | −12.7 | (5.52) | −7.9 | (6.83) | −10.5 | (5.06) |
| TRT Diff vs. PBO: p-value (Diff, 95% CI) | | | 0.515 (4.79, −10.10 to 19.67 | | 0.732 (2.19, −10.84 to 15.22 | |
| Week 6 Change from Baseline: N, Mean (SD) | 10, −13.5 | (17.08) | 6, −5.7 | (9.33) | 12, −14.8 | (15.77) |
| Week 6 Change from Baseline: LSMean (SE) | −14.9 | (5.95) | −7.4 | (7.45) | −14.7 | (5.45) |
| TRT Diff vs. PBO: p-value (Diff, 95% CI) | | | 0.367 (7.48, −9.26 to 24.22 | | 0.980 (0.18, −14.32 to 14.67 | |
| Week 9 Change from Baseline: N, Mean (SD) | 10, −11.9 | (14.26) | 6, −15.3 | (15.60) | 12, −15.3 | (13.79) |
| Week 9 Change from Baseline: LSMean (SE) | −13.4 | (5.69) | −17.7 | (7.09) | −15.2 | (5.15) |
| TRT Diff vs. PBO: p-value (Diff, 95% CI) | | | 0.577 (−4.28, −19.83 to 11.27 | | 0.789 (−1.76, −15.16 to 11.64 | |
| Week 12 Change from Baseline: N, Mean (SD) | 10, −13.2 | (19 | 6, −9.8 | (11.96) | 12, −16.2 | (15.32) |
| Week 12 Change from Baseline: LSMean (SE) | | | −11.5 | (8.03) | −16.0 | (5.77) |

| | | |
|---|---|---|
| TRT Diff vs. PBO: p-value (Diff, 95% CI) | 0.719 (3.24, −15.08 to 21.57 | 0.872 (−1.23, −16.86 to 14.40 |

TABLE 26U
CMAI Total Score: Change from Baseline SPCD MMRM, Observed Data
By Anticholinesterases Med Use
mITT Population and CMAI Factor 1 Agitated at Baseline
On Treatment Anticholinesterases Use: Yes

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 95, 78.5 | (23.58) | 41, 78.0 | (23.77) | 38, 74.6 | (24.86) |
| Week 1 Change from Baseline: N, Mean (SD) | 91, −6.9 | (9.13) | 39, −6.8 | (13.71) | 37, −9.9 | (11.15) |
| Week 1 Change from Baseline: LSMean (SE) | −5.6 | (1.64) | −6.1 | (2.08) | −9.4 | (2.07) |
| TRT Diff vs. PBO: p-value (Diff, 95% CI) | | | 0.775 (−0.57, −4.51 to 3.37) | | 0.060 (−3.87, −7.90 to 0.17) | |
| Week 2 Change from Baseline: N, Mean (SD) | 75, −9.7 | (12.07) | 30, −11.0 | (16.69) | 26, −3.9 | (12.15) |
| Week 2 Change from Baseline: LSMean (SE) | −7.9 | (1.74) | −9.1 | (2.31) | −8.7 | (2.35) |
| TRT Diff vs. PBO: p-value (Diff, 95% CI) | | | 0.597 (−1.22, −5.79 to 3.34) | | 0.731 (−0.82, −5.54 to 3.89) | |
| Week 3 Change from Baseline: N, Mean (SD) | 92, −11.2 | (14.10) | 39, −12.8 | (17.06) | 38, −11.3 | (12.75) |
| Week 3 Change from Baseline: LSMean (SE) | −9.9 | (1.85) | −11.8 | (2.48) | −11.3 | (2.48) |
| TRT Diff vs. PBO: p-value (Diff, 95% CI) | | | 0.463 (−1.88, −1.94 to 3.17) | | 0.594 (−1.39, −6.54 to 3.76) | |
| Week 6 Change from Baseline: N, Mean (SD) | 89, −14.2 | (15.19) | 38, −16.4 | (17.14) | 37, −14.5 | (12.92) |
| Week 6 Change from Baseline: LSMean (SE) | −12.7 | (1.87) | −15.8 | (2.52) | −14.7 | (2.52) |
| TRT Diff vs. PBO: p-value (Diff, 95% CI) | | | 0.250 (−3.02, −8.19 to 2.15) | | 0.466 (−1.94, −7.19 to 3.30) | |
| Stage 2 (Stage 1 Placebo Non-Responder Only) | | | | | | |
| Baseline: N, Mean (SD) | 21, 72.7 | (29.29) | 16, 67.6 | (18.20) | 24, 66.3 | (21.88) |
| Week 9 Change from Baseline: N, Mean (SD) | 21, −0.6 | (7.74) | 16, −9.1 | (11.42) | 24, −3.3 | (12.04) |
| Week 9 Change from Baseline: LSMean (SE) | 0.2 | (2.12) | −9.3 | (2.42) | −3.7 | (1.98) |
| TRT Diff vs. PBO: p-value (Diff, 95% CI) | | | 0.005 (−9.45, −15.89 to −3.00) | | 0.189 (−3.36, −9.68 to 1.96) | |
| Week 12 Change from Baseline: N, Mean (SD) | 21, −2.0 | (9.98) | 16, −8.3 | (15.51) | 23, −4.7 | (13.64) |
| Week 12 Change from Baseline: LSMean (SE) | −1.3 | (2.76) | −6.5 | (3.14) | −5.1 | (2.61) |

| | | |
|---|---|---|
| TRT Diff vs. PBO: p-value (Diff, 95% CI) | 0.225 (−5.14, −13.52 to 3.25) | 0.326 (−3.78, −11.42 to 3.86) |
| OLS Z-statistic, p-value | −1.634, p = 0.092 | −1.214, p = 0.225 |

TABLE 26V
CMAI Total Score: Change from Baseline SPCD MMRM, Observed Data
By Anticholinesterases Med Use
mITT Population and CMAI Factor 1 Agitated at Baseline
On Treatment Anticholinesterases Use: No

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 48, 78.6 | (20.84) | 23, 83.7 | (19.90) | 28, 85.9 | (20.93) |
| Week 1 Change from Baseline: N, Mean (SD) | 48, 7.7 | (11.68) | 23, −10.7 | (13.96) | 27, −5.8 | (8.77) |
| Week 1 Change from Baseline: LSMean (SE) | −10.9 | (2.75) | −13.0 | (3.11) | −7.7 | (2.97) |
| TRT Diff vs. PBO: p-value (Diff, 95% CI) | | | 0.462 (−2.13, −7.35 to 3.60) | | 0.240 (3.23, −2.19 to 8.86) | |
| Week 2 Change from Baseline: N, Mean (SD) | 39, −7.0 | (10.57) | 19, −13.0 | (16.29) | 24, −3.5 | (10.11) |
| Week 2 Change from Baseline: LSMean (SE) | −11.9 | (2.80) | −16.8 | (3.19) | −9.5 | (3.02) |
| TRT Diff vs. PBO: p-value (Diff, 95% CI) | | | 0.115 (−4.31, −10.82 to 1.19) | | 0.389 (2.47, −3.20 to 3.14) | |
| Week 3 Change from Baseline: N, Mean (SD) | 48, −12.0 | (14.22) | 22, −19.5 | (17.72) | 28, −10.3 | (13.92) |
| Week 3 Change from Baseline: LSMean (SE) | −15.8 | (2.99) | −20.7 | (3.56) | −11.6 | (3.33) |
| TRT Diff vs. PBO: p-value (Diff, 95% CI) | | | 0.166 (−4.97, −12.04 to 2.09) | | 0.218 (4.16, −2.47 to 10.30) | |
| Week 6 Change from Baseline: N, Mean (SD) | 48, −12.8 | (14.93) | 21, −18.7 | (18.56) | 27, −11.3 | (14.79) |
| Week 6 Change from Baseline: LSMean (SE) | −16.6 | (3.05) | −20.3 | (3.69) | −12.9 | (3.44) |
| TRT Diff vs. PBO: p-value (Diff, 95% CI) | | | 0.326 (−3.70, −11.14 to 3.74) | | 0.304 (3.63, −3.34 to 10.59) | |
| Stage 2 (Stage 1 Placebo Non-Responder Only) | | | | | | |
| Baseline: N, Mean (SD) | 8, 62.8 | (16.68) | 14, 73.2 | (16.28) | 11, 63.4 | (12.96) |
| Week 9 Change from Baseline: N, Mean (SD) | 8, 2.5 | (6.05) | 14, −2.5 | (7.50) | 11, −2.6 | (7.33) |
| Week 9 Change from Baseline: LSMean (SE) | 2.1 | (2.62) | −2.2 | (1.97) | −2.7 | (2.21) |
| TRT Diff vs. PBO: p-value (Diff, 95% CI) | | | 0.200 (−4.35, −11.13 to 2.43) | | 0.173 (−4.78, −11.78 to 2.21) | |
| Week 12 Change from Baseline: N, Mean (SD) | 8, 1.0 | (11.90) | 14, −4.6 | (11.29) | 11, −7.0 | (10.88) |
| Week 12 Change from Baseline: LSMean (SE) | −0.1 | (3.91) | −3.8 | (2.94) | −7.1 | (3.29) |

| | | |
|---|---|---|
| TRT Diff vs. PBO: p-value (Diff, 95% CI) | 0.456 (−3.73, −13.84 to 6.38) | 0.180 (−7.00, −17.44 to 3.43) |
| OLS Z-statistic, p-value | −1.239, p = 0.215 | −0.214, p = 0.831 |

TABLE 26W
CMAI: Change from Baseline Parallel Group MMRM Analysis - Observed Data
By Anticholinesterases Med Use
mITT 12-Week Parallel Group Population and CMAI Factor 1 Agitated at Baseline
On Treatment Anticholinesterases Use: Yes

| Parameter/Result | Placebo | | AVP-786 18 mg | | AVP-786 28 mg | |
|---|---|---|---|---|---|---|
| Baseline: N, Mean (SD) | 33, 77.3 | (25.19) | 41, 78.0 | (23.77) | 38, 74.6 | (24.86) |
| Week 1 Change from Baseline: N, Mean (SD) | 31, −6.8 | (8.15) | 39, −6.8 | (13.71) | 37, −9.9 | (11.15) |
| Week 1 Change from Baseline: LSMean (SE) | −3.8 | (2.38) | −4.5 | (2.28) | −8.2 | (2.25) |
| TRT Diff vs. PBO: p-value (Diff, 95% CI) | | | 0.801 (−0.67, −5.96 to 4.61) | | 0.103 (−4.43, −9.78 to 0.92) | |
| Week 2 Change from Baseline: N, Mean (SD) | 28, −9.5 | (12.12) | 30, −11.0 | (16.69) | 26, −8.9 | (12.15) |
| Week 2 Change from Baseline: LSMean (SE) | −7.1 | (2.58) | −7.7 | (2.49) | −7.4 | (2.49) |
| TRT Diff vs. PBO: p-value (Diff, 95% CI) | | | 0.839 (−0.61, −6.53 to 5.35) | | 0.901 (−0.39, −6.46 to 5.69) | |
| Week 3 Change from Baseline: N, Mean (SD) | 32, −10.8 | (16.63) | 39, −12.8 | (17.06) | 38, −11.3 | (12.75) |
| Week 3 Change from Baseline: LSMean (SE) | −7.7 | (2.85) | −9.9 | (2.70) | −9.9 | (2.69) |
| TRT Diff vs. PBO: p-value (Diff, 95% CI) | | | 0.518 (−2.22, −8.99 to 4.55) | | 0.522 (−2.21, −9.05 to 4.62) | |
| Week 6 Change from Baseline: N, Mean (SD) | 31, −12.0 | (15.63) | 38, −16.4 | (17.14) | 37, −14.5 | (12.82) |
| Week 6 Change from Baseline: LSMean (SE) | −9.0 | (2.81) | −13.9 | (2.65) | −13.3 | (2.63) |
| TRT Diff vs. PBO: p-value (Diff, 95% CI) | | | 0.140 (−4.97, −11.59 to 1.66) | | 0.205 (−4.30, −10.98 to 2.38) | |
| Week 9 Change from Baseline: N, Mean (SD) | 30, −12.8 | (17.23) | 38, −15.3 | (19.24) | 34, −13.1 | (16.30) |
| Week 9 Change from Baseline: LSMean (SE) | −10.0 | (3.10) | −13.4 | (2.89) | −12.3 | (2.92) |
| TRT Diff vs. PBO: p-value (Diff, 95% CI) | | | 0.370 (−3.39, −10.87 to 4.08) | | 0.549 (−2.30, −9.91 to 5.30) | |
| Week 12 Change from Baseline: N, Mean (SD) | 31, −13.5 | (16.70) | 38, −16.1 | (17.84) | 36, −12.9 | (12.88) |
| Week 12 Change from Baseline: LSMean (SE) | −10.6 | (2.89) | −14.0 | (2.72) | −12.2 | (2.72) |

| | | |
|---|---|---|
| TRT Diff vs. PBO: p-value (Diff, 95% CI) | 0.321 (−3.46, −10.35 to 3.42) | 0.644 (−1.63, −8.59 to 5.34) |

TABLE 26X
CMAI: Change from Baseline Parallel Group MMRM Analysis - Observed Data
By Anticholinesterases Med Use
mITT 12-Week Parallel Group Population and CMAI Factor 1 Agitated at Baseline
On Treatment Anticholinesterases Use: No

| Parameter/Result | Placebo | | AVP-786 18 mg | | AVP-786 28 mg | |
|---|---|---|---|---|---|---|
| Baseline: N, Mean (SD) | 13, 72.5 | (14.42) | 23, 83.7 | (19.90) | 28, 85.9 | (20.93) |
| Week 1 Change from Baseline: N, Mean (SD) | 13, −10.9 | (12.14) | 23, −10.7 | (13.96) | 27, −5.8 | (8.77) |
| Week 1 Change from Baseline: LSMean (SE) | −14.9 | (4.00) | −13.5 | (3.46) | −8.6 | (3.30) |
| TRT Diff vs. PBO: p-value (Diff, 95% CI) | | | 0.749 (1.34, −7.04 to 9.73) | | 0.127 (6.27, −1.83 to 14.36 | |
| Week 2 Change from Baseline: N, Mean (SD) | 10, −3.2 | (12.93) | 19, −13.0 | (16.29) | 24, −8.5 | (10.11) |
| Week 2 Change from Baseline: LSMean (SE) | −9.9 | (4.22) | −17.0 | (3.53) | −10.3 | (3.39) |
| TRT Diff vs. PBO: p-value (Diff, 95% CI) | | | 0.118 (−7.12, −16.11 to 1.36) | | 0.986 (−0.35, −9.03 to 8.33) | |
| Week 3 Change from Baseline: N, Mean (SD) | 13, −9.7 | (15.78) | 22, −19.5 | (17.72) | 28, −10.3 | (18.92) |
| Week 3 Change from Baseline: LSMean (SE) | −16.0 | (4.76) | −21.3 | (3.96) | −12.4 | (3.71) |
| TRT Diff vs. PBO: p-value (Diff, 95% CI) | | | 0.316 (−5.34, −15.91 to 5.23) | | 0.488 (3.56, −6.84 to 13.77 | |
| Week 6 Change from Baseline: N, Mean (SD) | 13, −12.8 | (16.32) | 21, −18.7 | (18.56) | 27, −11.3 | (14.79) |
| Week 6 Change from Baseline: LSMean (SE) | −19.0 | (4.91) | −20.9 | (4.09) | −13.8 | (3.81) |
| TRT Diff vs. PBO: p-value (Diff, 95% CI) | | | 0.735 (−2.88, −12.94 to 9.18) | | 0.336 (5.17, −5.49 to 15.82 | |
| Week 9 Change from Baseline: N, Mean (SD) | 12, −10.2 | (16.33) | 20, −24.4 | (18.27) | 28, −12.8 | (14.40) |
| Week 9 Change from Baseline: LSMean (SE) | −16.4 | (4.85) | −26.2 | (4.04) | −15.1 | (3.74) |
| TRT Diff vs. PBO: p-value (Diff, 95% CI) | | | 0.079 (−9.73, −20.61 to 1.15) | | 0.794 (1.37, −9.07 to 11.82 | |
| Week 12 Change from Baseline: N, Mean (SD) | 12, −11.2 | (21.04) | 21, −24.6 | (19.20) | 28, −14.3 | (17.07) |
| Week 12 Change from Baseline: LSMean (SE) | −13.5 | (5.42) | −26.3 | (4.40) | −16.8 | (4.05) |

| | | |
|---|---|---|
| TRT Diff vs. PBO: p-value (Diff, 95% CI) | 0.210 (−7.85, −30.26 to 4.56) | 0.779 (1.68, −10.16 to 13.63 |

TABLE 26Y
CMAI Total Score: Change from Baseline SPCD MMRM, Observed Data
By Memantine Use
mITT Population and CMAI Factor 1 Agitated at Baseline
On Treatment Memantine Use: Yes

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 71, 79.5 | (24.17) | 31, 82.6 | (22.57) | 34, 82.5 | (24.48) |
| Week 1 Change from Baseline: N, Mean (SD) | 70, −6.4 | (8.93) | 30, −12.8 | (17.46) | 32, −5.6 | (8.10) |
| Week 1 Change from Baseline: LSMean (SE) | −5.6 | (2.21) | −11.6 | (2.50) | −4.4 | (2.52) |
| TRT Diff vs. PBO: p-value (Diff, 95% CI) | | | 0.012 (−6.01, −10.68 to −1.35) | | 0.599 (1.21, −3.34 to 5.77) | |
| Week 2 Change from Baseline: N, Mean (SD) | 59, −7.8 | (10.44) | 28, −15.0 | (19.53) | 25, −6.3 | (9.93) |
| Week 2 Change from Baseline: LSMean (SE) | −6.7 | (2.23) | −14.1 | (2.65) | −4.8 | (2.70) |
| TRT Diff vs. PBO: p-value (Diff, 95% CI) | | | 0.005 (−7.36, −12.47 to −2.24) | | 0.463 (1.89, −3.18 to 6.96) | |
| Week 3 Change from Baseline: N, Mean (SD) | 68, −9.9 | (11.77) | 31, −19.4 | (19.45) | 34, −8.3 | (12.27) |
| Week 3 Change from Baseline: LSMean (SE) | −9.1 | (2.36) | −18.2 | (2.79) | −7.1 | (2.76) |
| TRT Diff vs. PBO: p-value (Diff, 95% CI) | | | 0.001 (−9.08, −14.62 to −3.55) | | 0.461 (2.01, −3.36 to 7.33) | |
| Week 6 Change from Baseline: N, Mean (SD) | 67, −12.8 | (13.15) | 31, −19.8 | (19.98) | 33, −10.2 | (12.60) |
| Week 6 Change from Baseline: LSMean (SE) | −12.0 | (2.43) | −18.6 | (2.90) | −9.3 | (2.89) |
| TRT Diff vs. PBO: p-value (Diff, 95% CI) | | | 0.026 (−6.64, −12.49 to −0.79) | | 0.353 (2.69, −3.02 to 8.40) | |
| Stage 2 (Stage 1 Placebo Non-Responder Only) | | | | | | |
| Baseline: N, Mean (SD) | 12, 74.8 | (35.78) | 17, 70.5 | (20.40) | 17, 65.1 | (23.92) |
| Week 9 Change from Baseline: N, Mean (SD) | 12, 1.0 | (8.38) | 17, −7.5 | (11.00) | 17, −3.1 | (13.26) |
| Week 9 Change from Baseline: LSMean (SE) | 2.0 | (2.98) | −7.2 | (2.49) | −3.9 | (2.51) |
| TRT Diff vs. PBO: p-value (Diff, 95% CI) | | | 0.021 (−9.29, −17.13 to −1.45) | | 0.139 (−5.91, −13.82 to 2.00) | |
| Week 12 Change from Baseline: N, Mean (SD) | 12, 3.3 | (7.56) | 17, −7.7 | (12.18) | 16, −7.0 | (14.46) |
| Week 12 Change from Baseline: LSMean (SE) | 4.1 | (5.30) | −7.5 | (2.76) | −7.8 | (2.82) |

| | | |
|---|---|---|
| TRT Diff vs. PBO: p-value (Diff, 95% CI) | 0.010 (−11.65, −20.32 to −2.98) | 0.009 (−11.90, −20.72 to −3.07) |
| OLS Z-statistic, p-value | −3.501, p = 0.001 | −1.277, p = 0.201 |

TABLE 26Z
CMAI Total Score: Change from Baseline SPCD MMRM, Observed Data
By Memantine Use
mITT Population and CMAI Factor 1 Agitated at Baseline
On Treatment Memantine Use: No

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 72, 77.6 | (21.11) | 33, 77.6 | (22.44) | 32, 76.1 | (22.94) |
| Week 1 Change from Baseline: N, Mean (SD) | 69, −7.9 | (11.10) | 32, −4.0 | (7.21) | 32, −10.9 | (11.73) |
| Week 1 Change from Baseline: LSMean (SE) | −7.7 | (1.80) | −4.6 | (2.32) | −11.3 | (2.23) |
| TRT Diff vs. PBO: p-value (Diff, 95% CI) | | | 0.169 (3.09, −1.33 to 7.50) | | 0.111 (−3.60, −8.05 to 0.85) | |
| Week 2 Change from Baseline: N, Mean (SD) | 55, −9.9 | (12.75) | 21, −7.5 | (9.84) | 25, −10.6 | (12.07) |
| Week 2 Change from Baseline: LSMean (SE) | −10.3 | (1.90) | −8.7 | (2.54) | −11.6 | (2.40) |
| TRT Diff vs. PBO: p-value (Diff, 95% CI) | | | 0.521 (1.63, −3.37 to 6.63) | | 0.599 (−1.32, −6.26 to 3.63) | |
| Week 3 Change from Baseline: N, Mean (SD) | 72, −13.0 | (15.92) | 30, −10.9 | (14.16) | 32, −13.7 | (13.70) |
| Week 3 Change from Baseline: LSMean (SE) | −13.2 | (2.09) | −10.3 | (2.84) | −14.5 | (2.75) |
| TRT Diff vs. PBO: p-value (Diff, 95% CI) | | | 0.417 (2.41, −3.45 to 8.27) | | 0.671 (−1.26, −7.11 to 4.60) | |
| Week 6 Change from Baseline: N, Mean (SD) | 70, −14.8 | (16.73) | 28, −14.3 | (14.15) | 31, −16.3 | (14.24) |
| Week 6 Change from Baseline: LSMean (SE) | −14.8 | (2.11) | −15.3 | (2.90) | −17.1 | (2.79) |
| TRT Diff vs. PBO: p-value (Diff, 95% CI) | | | 0.803 (−0.76, −6.77 to 5.25) | | 0.403 (−2.50, −8.45 to 3.45) | |
| Stage 2 (Stage 1 Placebo Non-Responder Only) | | | | | | |
| Baseline: N, Mean (SD) | 17, 66.5 | (17.91) | 13, 69.8 | (25.04) | 18, 68.7 | (16.24) |
| Week 9 Change from Baseline: N, Mean (SD) | 17, −0.2 | (6.74) | 13, −4.1 | (9.08) | 18, −3.1 | (8.18) |
| Week 9 Change from Baseline: LSMean (SE) | −0.4 | (1.92) | −4.0 | (2.19) | −3.0 | (1.86) |
| TRT Diff vs. PBO: p-value (Diff, 95% CI) | | | 0.226 (−3.58, −9.46 to 2.30) | | 0.329 (−2.64, −8.03 to 2.75) | |
| Week 12 Change from Baseline: N, Mean (SD) | 17, −4.2 | (11.21) | 13, −2.6 | (15.06) | 18, −4.1 | (11.13) |
| Week 12 Change from Baseline: LSMean (SE) | −4.6 | (2.39) | −2.3 | (3.30) | −4.0 | (2.80) |

| | | |
|---|---|---|
| TRT Diff vs. PBO: p-value (Diff, 95% CI) | 0.612 (2.24, −6.61 to 11.10) | 0.892 (0.55, −7.57 to 8.67) |
| OLS Z-statistic, p-value | 0.174, p = 0.861 | −0.529, p = 0.597 |

TABLE 26AA
CMAI: Change from Baseline Parallel Group MMRM Analysis - Observed Data
By Memantine Use
mITT 12-Week Parallel Group Population and CMAI Factor 1 Agitated at Baseline
On Treatment Memantine Use: Yes

| Parameter/Result | Placebo | | AVP-786 18 mg | | AVP-786 28 mg | |
|---|---|---|---|---|---|---|
| Baseline: N, Mean (SD) | 17, 79.2 | (30.29) | 31, 82.6 | (22.57) | 34, 82.5 | (24.43) |
| Week 1 Change from Baseline: N, Mean (SD) | 17, −4.8 | (7.57) | 30, −12.8 | (17.46) | 32, −5.6 | (8.10) |
| Week 1 Change from Baseline: LSMean (SE) | −5.1 | (3.99) | −12.3 | (3.10) | −5.4 | (3.18) |
| TRT Diff vs. PBO: p-value (Diff, 95% CI) | | | 0.053 (−7.22, −14.53 to 0.09) | | 0.928 (−0.33, −7.50 to 6.85) | |
| Week 2 Change from Baseline: N, Mean (SD) | 15, −3.2 | (7.59) | 28, −15.0 | (19.53) | 25, −6.8 | (9.95) |
| Week 2 Change from Baseline: LSMean (SE) | −3.6 | (4.17) | −15.2 | (3.21) | −5.8 | (3.32) |
| TRT Diff vs. PBO: p-value (Diff, 95% CI) | | | 0.005 (−11.51, −19.37 to −3.66 | | 0.573 (−2.21, −9.99 to 5.56) | |
| Week 3 Change from Baseline: N, Mean (SD) | 16, −4.7 | (8.87) | 31, −19.4 | (19.45) | 34, −8.3 | (12.27) |
| Week 3 Change from Baseline: LSMean (SE) | −4.4 | (4.32) | −19.1 | (3.31) | −8.1 | (3.36) |
| TRT Diff vs. PBO: p-value (Diff, 95% CI) | | | <0.001 (−14.69, −23.03 to −6.3 | | 0.369 (−5.71, −11.89 to 4.47) | |
| Week 6 Change from Baseline: N, Mean (SD) | 16, −8.2 | (12.12) | 31, −19.0 | (19.98) | 33, −10.2 | (12.50) |
| Week 6 Change from Baseline: LSMean (SE) | −8.0 | (4.50) | −19.5 | (3.42) | −10.3 | (3.46) |
| TRT Diff vs. PBO: p-value (Diff, 95% CI) | | | 0.012 (−11.51, −20.38 to −2.64 | | 0.602 (−2.29, −11.00 to 6.42) | |
| Week 9 Change from Baseline: N, Mean (SD) | 15, −7.2 | (13.81) | 29, −23.0 | (20.54) | 32, −9.5 | (15.00) |
| Week 9 Change from Baseline: LSMean (SE) | −7.8 | (4.63) | −22.7 | (3.50) | −9.9 | (3.53) |
| TRT Diff vs. PBO: p-value (Diff, 95% CI) | | | 0.002 (−14.39, −24.13 to −5.64 | | 0.636 (−2.16, −11.23 to 6.90) | |
| Week 12 Change from Baseline: N, Mean (SD) | 15, −5.1 | (14.46) | 30, −21.8 | (21.13) | 33, −11.7 | (16.40) |
| Week 12 Change from Baseline: LSMean (SE) | −5.8 | (4.92) | −21.7 | (3.69) | −11.9 | (3.70) |

| | | |
|---|---|---|
| TRT Diff vs. PBO: p-value (Diff, 95% CI) | 0.002 (−15.89, −25.98 to −5.80 | 0.223 (−6.12, −16.03 to 3.79) |

TABLE 26BB
CMAI: Change from Baseline Parallel Group MMPM Analysis - Observed Data By Memantine Use
mITT 12-Week Parallel Group Population and CMAI Factor 1 Agitated at Baseline
On Treatment Memantine Use: No

| Parameter/Result | Placebo | AVP-786 18 mg | AVP-786 28 mg |
|---|---|---|---|
| Baseline: N, Mean (SD) | 29, 74.1 (16.95) | 33, 77. [symbol] (22.44) | 32, 76.1 (22. [symbol] 4) |
| Week 1 Change from Baseline: N, Mean (SD) | 27, −10.1 (10.20) | 32, −4.0 (7.21) | 33, −10.3 (11.73) |
| Week 1 Change from Baseline: LSMean (SE) | −8. [symbol] (2.21) | −3.1 (2.24) | −10.0 (2.16) |
| TRT Diff vs. PBO: p-value (Diff, 95% CI) | | 0.057 (5.15, −0.15 to 10.44 | 0.505 (−1.79, −7.09 to 3.52) |
| Week 2 Change from Baseline: N, Mean (SD) | 23, -10.9 (14.18) | 21, −7.5 (9.84) | 25, −10.6 (12.07) |
| Week 2 Change from Baseline: LSMean (SE) | −10.5 (2.41) | − [symbol] .9 (2.50) | −10.3 (2.36) |
| TRT Diff vs. PBO: p-value (Diff, 95% CI) | | 0.237 (3.60, −2.41 to 9.61) | 0.924 (0.2 [symbol] , −5.64 to 6.21) |
| Week 3 Change from Baseline: N, Mean (SD) | 29, −13.6 (13.49) | 30, −10.9 (14.16) | 32, −13.7 (13.70) |
| Week 3 Change from Baseline: LSMean (SE) | −13. [symbol] (3.87) | −8.9 ( [symbol] .87) | −13.0 (2.7 [symbol] ) |
| TRT Diff vs. PBO: p-value (Diff, 95% CI) | | 0.250 (4.27, −3.06 to 11.60 | 0.954 (0.21, −7.08 to 7.51) |
| Week 6 Change from Baseline: N, Mean (SD) | 23, −14.6 (17.12) | 28, −14.3 (14.15) | 31, −16.3 (14.24) |
| week 6 Change from Baseline: LSMean (SE) | −13.9 (2. [symbol] 5) | −13.6 (2. [symbol] 7) | −15.7 ( [symbol] .76) |
| TRT Diff vs. PBO: p-value (Diff, 95% CI) | | 0.924 (0.35, −6.94 to 7.64) | 0.627 (−1.77, −9.00 to 5.45) |
| Week 9 Change from Baseline: N, Mean (SD) | 27, −14.7 (27.96) | 29, −13.9 (17.01) | 30, −16.6 (15.11) |
| Week 9 Change from Baseline: LSMean (SE) | −14.4 (3.10) | −13.2 (3.09) | −15.9 (2.99) |
| TRT Diff vs. PBO: p-value (Diff, 95% CI) | | 0.778 (1.14, −6.89 to 9.17) | 0.710 (−1.50, −9.46 to 6.47) |
| Week 12 Change from Baseline: N, Mean (SD) | 28, −16.9 (1 [symbol] .26) | 29, −16.4 (15.52) | 31, −16.0 (12.62) |
| Week 12 Change from Baseline: LSMean (SE) | −16.3 (2.8 [symbol] ) | -15.1 (2.92) | −15.3 (2.78) |
| TRT Diff vs. PBO: p-value (Diff, 95% CI) | | 0.741 (1.24, −6.21 to [symbol] .69) | 0.792 (0.97, −6.35 to 6.30) |

[symbol]

indicates data missing or illegible when filed

TABLE 26CC
CMAI Total Score: Change from Baseline SPCD MMPM, Observed Data By Benzodiazepines Use
mITT Population and CMAI Factor 1 Agitated at Baseline
On Treatment Benzodiazepines Use: Yes

| Stage/ | | | |
|---|---|---|---|
| Parameter/Result | Placebo | AVP-786 18 mg | AVP-786 28 mg |
| Stage 1 | | | |
| Baseline: N, Mean (SD) | 40, 85. [symbol] (28.46) | 18, 91.9 (1 [symbol] . [symbol] 2) | 14, [symbol] .4 (22.08) |
| Week 1 Change from Baseline: N, Mean (SD) | 40, −8.1 (11.49) | 18, −9.7 13.71 | 14, −0.6 6.05 |
| Week 1 Change from Baseline: LSMean (SE) | −12.9 (3.11) | −12. [symbol] (3.54) | −4.3 (3. [symbol] ) |
| TRT Diff vs. PBO: p-value (Diff, 95% CI) | | 0.900, −0.99 −6.49 to 5.71 | 0.035, 7.12 0.50 to 13.73 |
| Week 2 Change from Baseline: N, Mean (SD) | 30, −9.7 (14.34) | 16, −15.9 (19.01) | 10, −2.3 (8.82) |
| Week 2 Change from Baseline: LSMean (SE) | −15.1 (3.40) | −17.8 (4.03) | −7.1 (4.49) |
| TRT Diff vs. PBO: p-value (Diff, 95% CI) | | 0.489 (−3.69, −10.41 to 5.04) | 0.065 (8.04, −0.53 to 16.60) |
| Week 3 Change from Baseline: N, Mean (SD) | 39, −13.4 (17.41) | 18, −21.6 (18.46) | 14, −6.4 (10.57) |
| Week 3 Change from Baseline: LSMean (SE) | −17.5 ( [symbol] .57) | −23.7 (4.38) | −11.0 (4.81) |
| TRT Diff vs. PBO: p-value (Diff, 95% CI) | | 0.160 (−6.22, −14.95 to 2.51) | 0.173 (6.54, −2.94 to 16.01) |
| Week 6 Change from Baseline: N, Mean (SD) | 38, −16.4 (18.00) | 18, −23.5 (19.90) | 13, −6.6 (1 [symbol] .67) |
| week 6 Change from Baseline: LSMean (SE) | −20.3 (3.62) | −25.4 (4.46) | −11.8 (4.96) |
| TRT Diff vs. PBO: p-value (Diff, 95% CI) | | 0.262 (−5.09, −14.08 to 3.89) | 0.089 (8.52, −1.34 to 18.39) |
| Stage 2 (Stage 1 Placebo Non-Responder Only) | | | |
| Baseline: N, Mean (SD) | 8, 74.8 (42.93) | 8, 79.2 (28.71) | 11, 61.5 (13.79) |
| Week 9 Change from Baseline: N, Mean (SD) | 8, 5.0 (3.91) | 8, −3.4 (7.83) | 11, −3.0 (10.15) |
| Week 9 Change from Baseline: LSMean (SE) | 5.4 (3.06) | −2.5 (2.91) | −4.1 (2.66) |
| TRT Diff vs. PBO: p-value (Diff, 95% CI) | | 0.071 (−7.93, −16.60 to 0.73) | 0.028 (−9.54, −17.97 to −1.12) |
| Week 12 Change from Baseline: N, Mean (SD) | 8, 9.0 (3.34) | 9, −3.7 (18.11) | 11, −3.5 (10.95) |
| Week 12 Change from Baseline: LSMean (SE) | 9.4 (3.84) | −2.8 (3.67) | −4.5 (3.34) |
| TRT Diff vs. PBO: p-value (Diff, 95% CI) | | 0.030 (−12.31, −23.10 to −1.32) | 0.012 (−13.94, −24.53 to −3.34) |
| OLS Z-statistic, p-value | | −2.316, p = 0.021 | −0.128, p = 0.898 |

[symbol]

indicates data missing or illegible when filed

TABLE 26DD
CMAI Total Score: Change from Baseline SPCD MMPM, Observed Data By Benzodiazepines Use
mITT Population and CMAI Factor 1 Agitated at Baseline
On Treatment Benzodiazepines Use: No

| Stage/ | | | |
|---|---|---|---|
| Parameter/Result | Placebo | AVP-786 18 mg | AVP-786 28 mg |
| Stage 1 | | | |
| Baseline: N, Mean (SD) | 103, 75.8 (19.38) | 46, 75.4 (22.23) | 52, 78.3 (24.31) |
| Week 1 Change from Baseline: N, Mean (SD) | 99, −6.7 (9.44) | 44, −7.6 (13.97) | 50, −10.3 (10.35) |
| Week 1 Change from Baseline: LSMean (SE) | −5.8 (1.62) | −7.3 (1.99) | −9.3 (1.89) |
| TRT Diff vs. PBO: p-value (Diff, 95% CI) | | 0.446 (−1.47, −5.26 to 2.32) | 0.062 (−3.47, −7.11 to 0.17) |
| Week 2 Change from Baseline: N, Mean (SD) | 84, −8.5 (10.54) | 33, −9.8 (14. [symbol] 7) | 40, −10.3 (11.14) |
| Week 2 Change from Baseline: LSMean (SE) | −7.4 (1.64) | −9.7 (2.06) | − [symbol] .0 (1.95) |
| TRT Diff vs. PBO: p-value (Diff, 95% CI) | | 0.267 (−2.26, −6.26 to 1.74) | 0.413 (−1.58, −5.39 to 2.32) |
| Week 3 Change from Baseline: N, Mean (SD) | 101, −10.8 (12.61) | 46, −12.5 (16.03) | 52, −12.1 (13.63) |
| Week 3 Change from Baseline: LSMean (SE) | −10.0 (1.76) | −11.8 (2.25) | −11.0 (2.11) |
| TRT Diff vs. PBO: p-value (Diff, 95% CI) | | 0.451 (−1.74, −6.28 to 2.80) | 0.648 (−1.00, −5.32 to 3.32) |
| Week 6 Change from Baseline: N, Mean (SD) | 99, −127−.7 (13.73) | 41, −14.5 (1 [symbol] .87) | 51, −14.8 (13.28) |
| week 6 Change from Baseline: LSMean (SE) | −11.9 (1.77) | −14.4 (2.31) | −13.8 (2.14) |
| TRT Diff vs. PBO: p-value (Diff, 95% CI) | | 0.285 (−2.34, −7.23 to 2.14) | 0.372 (−2.00, −6.41 to 2.41) |
| Stage 2 (Stage 1 Placebo Non-Responder Only) | | | |
| Baseline: N, Mean (SD) | 21, 68.1 (28.07) | 21, 66.4 (18.13) | 24, 69.5 (22.24) |
| Week 9 Change from Baseline: N, Mean (SD) | 21, −1.5 (5.95) | 21, −4.2 (11.04) | 24, −3.1 (11.47) |
| Week 9 Change from Baseline: LSMean (SE) | −1.5 (1.37) | −7.4 (1.97) | −2.7 (1.84) |
| TRT Diff vs. PBO: p-value (Diff, 95% CI) | | 0.037 (−3.92, −11.43 to −9.36) | 0.636 (−1.28, −6.68 to 4.10) |
| Week 12 Change from Baseline: N, Mean (SD) | 21, −5.0 (8.40) | 21, −6.3 (13.91) | 2 [symbol] , −6.4 (13.58) |
| Week 12 Change from Baseline: LSMean (SE) | −4.9 (2.48) | −8.8 (2.48) | − [symbol] .2 (2.35) |
| TRT Diff vs. PBO: p-value (Diff, 95% CI) | | 0.629 (−1.70, −8.72 to 5.31) | 0.726 (−1.20, −3.03 to 5.63) |
| OLS Z-statistic, p-value | | −1.104, p = 0.270 | −0.877, p = 0.380 |

[symbol]

indicates data missing or illegible when filed

TABLE 26EE
CMAI: Change from Baseline Parallel Group MMPM Analysis - Observed Data By Benzodiazepines Use
mITT 12-Week Parallel Group Population and CMAI Factor 1 Agitated at Baseline
On Treatment Benzodiazepines Use: Yes

| Parameter/Result | Placebo | AVP-786 18 mg | AVP-786 28 mg |
|---|---|---|---|
| Baseline: N, Mean (SD) | 12, 86.5 (36.93) | 18, 91.9 (18.81) | 14, 33.4 (20.08) |
| Week 1 Change from Baseline: N, Mean (SD) | 12, − [symbol] .9 (7.68) | 18, −9.7 (13.71) | 14, −0.6 (6.05) |
| Week 1 Change from Baseline: LSMean (SE) | −13.0 (3.96) | −15.6 (3.74) | −7.9 (4.02) |
| TRT Diff vs. PBO: p-value (Diff, 95% CI) | | 0.485 (−2.60, −10.08 to 4.87) | 0.201 (5.07, −2.83 to 12.97 |
| Week 2 Change from Baseline: N, Mean (SD) | 11, −8.4 (16.07) | 16, −15.9 (19.0 [symbol] ) | 10, −2. [symbol] (8.82) |
| Week 2 Change from Baseline: LSMean (SE) | −15.6 (4.80) | −20.7 (4.37) | −9.7 (4.85) |
| TRT Diff vs. PBO: p-value (Diff, 95% CI) | | 0.318 (−5.17, −15.53 to 4.19) | 0.293 ( 5.84, −5.25 to 16.93 |
| Week 3 Change from Baseline: N, Mean (SD) | 12, −10.0 (21.84) | 18, −21.6 (19.46) | 14, − [symbol] .4 (10.57) |
| Week 3 Change from Baseline: LSMean (SE) | −16.3 (5.42) | −26.9 (4.82) | −14.3 (5.29) |
| TRT Diff vs. PBO: p-value (Diff, 95% CI) | | 0.087 (−10.62, −22.85 to 1.62) | 0.765 (1.92, −10.98 to 14. [symbol] 2 |
| Week 6 Change from Baseline: N, Mean (SD) | 12, −15.3 (18.36) | 18, −23.5 (19.90) | 13, −6.6 (13.67) |
| week 6 Change from Baseline: LSMean (SE) | −21.5 (5.44) | −28.8 (4.83) | −15.9 (5.34) |
| TRT Diff vs. PBO: p-value (Diff, 95% CI) | | 0.233 (−7.36, −1 [symbol] .67 to 4.94) | 0.390 (5.60, −7.42 to 13.62 |
| Week 9 Change from Baseline: N, Mean (SD) | 12, −12.1 (21.76) | 1 [symbol] , −25.5 (18.95) | 14, −9.5 (17.20) |
| Week 9 Change from Baseline: LSMean (SE) | −19.5 (5.43) | −30.0 (4.87) | −17.3 (5.30) |
| TRT Diff vs. PBO: p-value (Diff, 95% CI) | | 0.066 (−11.54, −23.88 to 0.80) | 0.918 ( 0.67, −12.26 to 13.5 [symbol] |
| Week 12 Change from Baseline: N, Mean (SD) | 12, −10.6 (26.31) | 17, −27.8 (22.67) | 14, −13.6 (20.06) |
| Week 12 Change from Baseline: LSMean (SE) | −17.0 (6.52) | −33.3 (8.71) | −22.0 (6.28) |
| TRT Diff vs. PBO: p-value (Diff, 95% CI) | | 0.040 (−16.37, −31.92 to −0.81 | 0.538 (−5.01, −21.34 to 11. [symbol] 1 |

[symbol]

indicates data missing or illegible when filed

TABLE 26FF
CMAI: Change from Baseline Parallel Group MMPM Analysis - Observed Data By Benzodiazepines Use
mITT 12-Week Parallel Group Population and CMAI Factor 1 Agitated at Baseline
On Treatment Benzodiazepines Use: No

| Parameter/Result | Placebo | AVP-786 18 mg | AVP-786 28 mg |
|---|---|---|---|
| Baseline: N, Mean (SD) | 34, 72.3 (13.72) | 46, 75.4 (22.23) | 52, 78.3 (24.31) |
| Week 1 Change from Baseline: N, Mean (SD) | 32, −3.4 (10.23) | 44, −7.6 (13.97) | 50, −10.3 (10.35) |
| Week 1 Change from Baseline: LSMean (SE) | −6.6 (2.3 [symbol] ) | −6.4 (2.17) | − [symbol] .4 (2.07) |
| TRT Diff vs. PBO: p-value (Diff, 95% CI) | | 0.940 (0.20, −5.05 to 5.45) | 0.502 (−1.75, −6.89 to 3.39) |
| Week 2 Change from Baseline: N, Mean (SD) | 27, −7.6 (21.06) | 33, −9.3 (14.37) | 40, −10.3 (12.14) |
| Week 2 Change from Baseline: LSMean (SE) | −6.9 (2.40) | −8.9 (2.21) | −8.1 (2.09) |
| TRT Diff vs. PBO: p-value (Diff, 95% CI) | | 0.475 (−1.93, −7.26 to 3.40) | 0.970 (−1.12, −6.31 to 4.07) |
| Week 3 Change from Baseline: N, Mean (SD) | 33, −10.6 (14.06) | 43, −12.5 (16.03) | 52, −12.1 (13.63) |
| Week 3 Change from Baseline: LSMean (SE) | −9.4 (2.67) | −10.7 (2.42) | −9. [symbol] (2.28) |
| TRT Diff vs. PBO: p-value (Diff, 95% CI) | | 0.974 (−1.31, −7.4 [symbol] to 4.82) | 0.860 (−0.53, − [symbol] .49 to 5.43) |
| Week 6 Change from Baseline: N, Mean (SD) | 32, −11.2 (14.68) | 41, −14.5 (15.87) | 51, −14.8 (13.28) |
| week 6 Change from Baseline: LSMean (SE) | −9.9 (2.69) | −13.3 (2.44) | −12.8 (2.29) |
| TRT Diff vs. PBO: p-value (Diff, 95% CI) | | 0.279 (−3.4 [symbol] , −9. [symbol] 4 to 2.77) | 0.387 (−2.9 [symbol] , − [symbol] .96 to 3.09) |
| Week 9 Change from Baseline: N, Mean (SD) | 30, −12.0 (14.84) | 42, −15.8 (18. [symbol] ) | 48, −14.0 (14.81) |
| Week 9 Change from Baseline: LSMean (SE) | −11.4 (2.95) | −14.3 (2.63) | −12.0 (2.48) |
| TRT Diff vs. PBO: p-value (Diff, 95% CI) | | 0.319 (−3.51, −10.45 to 3.43) | 0.857 (−0.61, −7.37 to 6.14) |
| Week 12 Change from Baseline: N, Mean (SD) | 31, −13.7 (13.65) | 42, −15.6 (13.6 [symbol] ) | 50, −13.8 (13.13) |
| Week 12 Change from Baseline: LSMean (SE) | −12.7 (2.64) | −14.4 (2.39) | −12.1 (2.23) |
| TRT Diff vs. PBO: p-value (Diff, 95% CI) | | 0.530 (−1.70, −7.75 to 4.35) | 0. [symbol] 34 (0.62, −5.03 to 6.47) |

[symbol]

indicates data missing or illegible when filed

TABLE 26GG
CMAI Total Score: Change from Baseline SPCD MMPM, Observed Data By Antidepressants Use
mITT Population and CMAI Factor 1 Agitated at Baseline
On Treatment Antidepressant Use: Yes

| Stage/ | | | |
|---|---|---|---|
| Parameter/Result | Placebo | AVP-786 18 mg | AVP-786 28 mg |
| Stage 1 | | | |
| Baseline: N, Mean (SD) | 96, 80.3 (23.44) | 46, [symbol] 3.7 (23.25) | 46, 79.7 (25.40) |
| Week 1 Change from Baseline: N, Mean (SD) | 9 [symbol] , −7.9 (10.39) | 45, −8.3 (14.13) | 44, −6.9 (9.08) |
| Week 1 Change from Baseline: LSMean (SE) | −6.7 (41.68) | −7.6 (2.05) | −6.6 (1.9 [symbol] ) |
| TRT Diff vs. PBO: p-value (Diff, 95% CI) | | 0.648 (−0.90, −4.79 to 2.99) | 0. [symbol] (0.10, −3.80 to 4.00) |
| Week 2 Change from Baseline: N, Mean (SD) | 79, −9.0 (11.99) | 38, −12.5 (16.23) | 33, −8.2 (8.96) |
| Week 2 Change from Baseline: LSMean (SE) | −9.2 (1.76) | −12.2 (2.20) | −7.8 (2.16) |
| TRT Diff vs. PBO: p-value (Diff, 95% CI) | | 0.166 (−3.05, −7.38 to 1.28) | 0.573 (1.25, −3.13 to 5.63) |
| Week 3 Change from Baseline: N, Mean (SD) | 94, −12.5 (14.1 [symbol] ) | 44, −16.1 (17.66) | 46, −9.9 (11.99) |
| Week 3 Change from Baseline: LSMean (SE) | −12.1 (1.88) | −14.8 (2.40) | −9.9 (2.33) |
| TRT Diff vs. PBO: p-value (Diff, 95% CI) | | 0.381 (−2.69, −7.58 to 2.22) | 0.377 (2.16, −2.69 to 7.06) |
| Week 6 Change from Baseline: N, Mean (SD) | 93, −14.6 (15.14) | 43, −18.7 (18.00) | 45, −11.6 (11.93) |
| week 6 Change from Baseline: LSMean (SE) | −14.2 (1.99) | −17.5 (2.44) | −11.9 (2.36) |
| TRT Diff vs. PBO: p-value (Diff, 95% CI) | | 0.297 (−3.29,, −8.30 to 1.72) | 0.366 (2.28, −2.68 to 7.24) |
| Stage 2 (Stage 1 Placebo Non-Responder Only) | | | |
| Baseline: N, Mean (SD) | 21, 73.1 (29.75) | 19, 69.7 (19.80) | 25, [symbol] .2 (22.52) |
| Week 9 Change from Baseline: N, Mean (SD) | 21, −0.9 (7.47) | 19, −4.3 (6.27) | 25, −2.8 (10.97) |
| Week 9 Change from Baseline: LSMean (SE) | −0.4 (1.67) | −4.3 (1.75) | −3.1 (1.53) |
| TRT Diff vs. PBO: p-value (Diff, 95% CI) | | 0.107 (−3.96, −9.90 to 0.98) | 0.230 (−2.75, −7.28 to 1.78) |
| Week 12 Change from Baseline: N, Mean (SD) | 21, −2.4 (20.27) | 19, −0.3 (13.31) | 24, −4.9 (12.35) |
| Week 12 Change from Baseline: LSMean (SE) | −1.9 (2.30) | −0.4 (2.62) | −5.3 (2.33) |
| TRT Diff vs. PBO: p-value (Diff, 95% CI) | | 0.674 (1.54, −5.71 to 8.79) | 0.328 (−3.39, −10.23 to 3.48) |
| OLS Z-statistic, p-value | | −0.646, p = 0.51 [symbol] | 0.008, p = 0.993 |

[symbol]

indicates data missing or illegible when filed

TABLE 26HH
CMAI Total Score: Change from Baseline SPCD MMPM, Observed Data By Antidepressants Use
mITT Population and CMAI Factor 1 Agitated at Baseline
On Treatment Antidepressant Use: No

| Stage/ | | | |
|---|---|---|---|
| Parameter/Result | Placebo | AVP-786 18 mg | AVP-786 28 mg |
| Stage 1 | | | |
| Baseline: N, Mean (SD) | 47, 74.9 (20.61) | 18, 70.7 (17.59) | 20, 7 [symbol] .8 (20.15) |
| Week 1 Change from Baseline: N, Mean (SD) | 46, −8.7 (9.64) | 17, −8.2 (13.38) | 20, −11.0 (12.52) |
| Week 1 Change from Baseline: LSMean (SE) | −7.5 (2.87) | −10.0 (3.33) | −10.6 (3.45) |
| TRT Diff vs. PBO: p-value (Diff, 95% CI) | | 0.414 (−2.49, −8.55 to 3.56) | 0.287 (−3.06, − [symbol] .76 to 2.63) |
| Week 2 Change from Baseline: N, Mean (SD) | 35, −8.3 (10.34) | 11, −9.5 (17.53) | 17, −9.5 (14.70) |
| Week 2 Change from Baseline: LSMean (SE) | −8.4 (2.97) | −9.4 (3.61) | −8.7 (3.61) |
| TRT Diff vs. PBO: p-value (Diff, 95% CI) | | 0.784 (−0.95, −7.82 to 5.92) | 0.925 (−0.30, −6.59 to 5.99) |
| Week 3 Change from Baseline: N, Mean (SD) | 46, −9.5 (13.9 [symbol] ) | 17, −12.8 (17.19) | 20, −13.1 (15.66) |
| Week 3 Change from Baseline: LSMean (SE) | −10.3 (3.09) | −14.9 (3.80) | −12.1 (3.87) |
| TRT Diff vs. PBO: p-value (Diff, 95% CI) | | 0.223 (−4.8 [symbol] , −12.08 to 2.86) | 0. [symbol] 22 (−1.76, −8.33 to 5.31) |
| Week 6 Change from Baseline: N, Mean (SD) | 44, −11.8 (14.38) | 16, −13. [symbol] (16.0 [symbol] ) | 19, −16. [symbol] (16.39) |
| week 6 Change from Baseline: LSMean (SE) | −12.5 (3.18) | −16.3 (4.03) | −15.3 (4.04) |
| TRT Diff vs. PBO: p-value (Diff, 95% CI) | | 0.348 (−3.82, −11.68 to 4.24) | 0.393 (−3.29, −10.91 to 4.33) |
| Stage 2 (Stage 1 Placebo Non-Responder Only) | | | |
| Baseline: N, Mean (SD) | [symbol] , 62.6 (13.24) | 11, 71.0 (26.69) | 10, 63.3 (12.70) |
| Week 9 Change from Baseline: N, Mean (SD) | 8, 3.3 (6.52) | 11, −9.0 (14.65) | 10, −3.7 (11. [symbol] 2) |
| Week 9 Change from Baseline: LSMean (SE) | 3.0 (4.29) | −3.7 (3.68) | −3.8 (3.91) |
| TRT Diff vs. PBO: p-value (Diff, 95% CI) | | 0.052 (−11.66, −23.41 to 0.09) | 0.245 (−8.81, −18.57 to 4.95) |
| Week 12 Change from Baseline: N, Mean (SD) | 8, 2.1 (10.74) | 11, −14.5 ( [symbol] .39) | 10, − [symbol] .8 (14.08) |
| Week 12 Change from Baseline: LSMean (SE) | 1.5 (3.98) | −13.8 ( [symbol] .41) | −7.1 (3.54) |
| TRT Diff vs. PBO: p-value (Diff, 95% CI) | | 0.008 (−15.31, −26.21 to −4.41) | 0.117 (−8.62, −1 [symbol] .53 to 2.30) |
| OLS Z-statistic, p-value | | −2.611, p = 0.009 | −1.735, p = 0.083 |

[symbol]

indicates data missing or illegible when filed

TABLE 26II
CMAI: Change from Baseline Parallel Group MMPM Analysis - Observed Data By Antidepressants Use
mITT 12-Week Parallel Group Population and CMAI Factor 1 Agitated at Baseline
On Treatment Antidepressant Use: Yes

| Parameter/Result | Placebo | AVP-786 18 mg | AVP-786 28 mg |
|---|---|---|---|
| Baseline: N, Mean (SD) | 34, 79.0 (24.33) | 46, 83.7 (23.25) | 46, 79.7 (25.40) |
| Week 1 Change from Baseline: N, Mean (SD) | 32, −3.2 (9.66) | 45, −8.5 (14.12) | 44, −6.9 (9.08) |
| Week 1 Change from Baseline: LSMean (SE) | −7.0 (2.33) | −7.2 (2.21) | −6.3 (2.12) |
| TRT Diff vs. PBO: p-value (Diff, 95% CI) | | 0.919 (−0.27, −5.42 to 4.89) | 0.793 (0.67, −4.46 to 5.80) |
| Week 2 Change from Baseline: N, Mean (SD) | 27, −9.1 (12.31) | 38 −12.5 (16.23) | 33, −8.2 (8.96) |
| Week 2 Change from Baseline: LSMean (SE) | −9.7 (2.43) | −11.9 (2.33) | −7.6 (2.27) |
| TRT Diff vs. PBO: p-value (Diff, 95% CI) | | 0.433 (−2.22, −7.82 to 3.37) | 0.472 (2.04, −3.56 to 7.65) |
| Week 3 Change from Baseline: N, Mean (SD) | 33, −11.7 (14.44) | 44, −16.1 (17.66) | 46, −9.96 (12.99) |
| Week 3 Change from Baseline: LSMean (SE) | −10.8 (2.76) | −14.3 (2.56) | −9.5 (2.47) |
| TRT Diff vs. PBO: p-value (Diff, 95% CI) | | 0.283 (−3.51, −93.95 to 2.94) | 0.682 (1.30, −5.09 to 7.89) |
| Week 6 Change from Baseline: N, Mean (SD) | 32, −13.0 (15.23) | 43, −16.7 (19.00) | 45, −11.6 (11.93) |
| week 6 Change from Baseline: LSMean (SE) | −12.2 (2.77) | −17.0 (2.57) | −11.4 (2.47) |
| TRT Diff vs. PBO: p-value (Diff, 95% CI) | | 0.140 (−4.86, −11.35 to 1.82) | 0.822 (0.73, −5.69 to 7.15) |
| Week 9 Change from Baseline: N, Mean (SD) | 30, −13.4 (16.24) | 42, −19.1 (20.04) | 4,3 −10.7 (14.12) |
| Week 9 Change from Baseline: LSMean (SE) | −12.9 (3.04) | −17.7 (2.77) | −11.2 (2.69) |
| TRT Diff vs. PBO: p-value (Diff, 95% CI) | | 0.195 (−4.76, −12.00 to 2.47) | 0.633 (1.74, −5.45 to 3.92) |
| Week 12 Change from Baseline: N, Mean (SD) | 31, −14.7 (18.42) | 43, −19.9 (18.89) | 44, −12.0 (14.69) |
| Week 12 Change from Baseline: LSMean (SE) | −14.0 (3.01) | −18.6 (2.74) | −11.3 (2.65) |
| TRT Diff vs. PBO: p-value (Diff, 95% CI) | | 0.207 (−4.53, −11.73 to 2.54) | 0.633 (1.71, −5.38 to 8.81) |

TABLE 26JJ
CMAI: Change from Baseline Parallel Group MMPM Analysis - Observed Data By Antidepressants Use
mITT 12-Week Parallel Group Population and CMAI Factor 1 Agitated at Baseline
On Treatment Antidepressant Use: No

| Parameter/Result | Placebo | AVP-786 18 mg | AVP-786 28 mg |
|---|---|---|---|
| Baseline: N, Mean (SD) | 12, 67.4 (14.39) | 18, 70.7 (17.59) | 20, 78.8 (20.25) |
| Week 1 Change from Baseline: N, Mean (SD) | 12, −7.7 (9.63) | 17, −8.2 (13.38) | 20, −11.0 (12.52) |
| Week 1 Change from Baseline: LSMean (SE) | −11.5 (5.28) | −11.3 (4.16) | −11.5 ( [symbol] .52) |
| TRT Diff vs. PBO: p-value (Diff, 95% CI) | | 0.965 (0.20, −3.92 to 9.32) | 0.996 (−0.02, −9.09 to 9.05) |
| Week 2 Change from Baseline: N, Mean (SD) | 12, −4.6 (12.92) | 12, −9.5 (17.53) | 27, −9.5 (14.70) |
| Week 2 Change from Baseline: LSMean (SE) | −8.5 (5.54) | −10.9 (4.51) | −9.3 (4.74) |
| TRT Diff vs. PBO: p-value (Diff, 95% CI) | | 0.6 [symbol] 3 (−2.34, −12.76 to 8.07) | 0.882 (−0.7 [symbol] , −10.97 to 9.36) |
| Week 3 Change from Baseline: N, Mean (SD) | 12, −7.0 (20.63) | 17, −12.8 (17.19) | 20, −13.1 (15.66) |
| Week 3 Change from Baseline: LSMean (SE) | −12.7 (5.09) | −16.2 (4.64) | −12.1 (4. [symbol] ) |
| TRT Diff vs. PBO: p-value (Diff, 95% CI) | | 0.541 (−3.48, −14.85 to 7.90) | 0.924 (0.54, −10.75 to 11.82) |
| Week 6 Change from Baseline: N, Mean (SD) | 12, −10.3 (17.25) | 16, −13.3 (16.08) | 19 −16.8 (16.89) |
| week 6 Change from Baseline: LSMean (SE) | −15.0 ( [symbol] .05) | −17.3 (4.83) | −17.1 (5.09) |
| TRT Diff vs. PBO: p-value (Diff, 95% CI) | | 0.697 (−2.33, −14.35 to 9.68) | 0.722 (−2.13, −14.02 to 9.79) |
| Week 9 Change from Baseline: N, Mean (SD) | 12, −8.5 (18.45) | 16, −16.8 (18.48) | 19, −18.0 (17.29) |
| Week 9 Change from Baseline: LSMean (SE) | −14.3 (5.95) | −21.3 (4.73) | −16.0 (5.02) |
| TRT Diff vs. PBO: p-value (Diff, 95% CI) | | 0.229 (−7.05, −13.72 to 4.61) | 0.762 (−1.75, −13.33 to 9.83) |
| Week 12 Change from Baseline: N, Mean (SD) | 12, −7.9 (20.87) | 16, −16.9 (18.30) | 20, −17.7 (14.41) |
| Week 12 Change from Baseline: LSMean (SE) | −13.0 (6.16) | −20.7 (4.96) | −17.3 (5.14) |
| TRT Diff vs. PBO: p-value (Diff, 95% CI) | | 0.213 (−7.72, −20.16 to 4.72) | 0.482 (−4.27, −16.49 to 7.95) |

[symbol]

indicates data missing or illegible when filed

TABLE 26KK
CMAI Total Score: Change from Baseline SPCD MMPM, Observed Data By CMAI Factor 1 Agitation Status
mITT Population and CMAI Factor 1 Agitated at Baseline
Agressive Behavior (Factor 1): Agitated

| Stage/ | | | |
|---|---|---|---|
| Parameter/Result | Placebo | AVP-786 18 mg | AVP-786 28 mg |
| Stage 1 | | | |
| Baseline: N, Mean (SD) | 143, 78.5 (22.62) | 84, 80.1 (22.46) | 66, 79.4 (23.78) |
| Week 1 Change from Baseline: N, Mean (SD) | 139, −7.1 (10.05) | 62, −8.2 (13.81) | 69, −8.2 (10.35) |
| Week 1 Change from Baseline: LSMean (SE) | −7.1 (1.41) | −8.3 (1.72) | −8.1 (1.68) |
| TRT Diff vs. PBO: p-value (Diff, 95% CI) | | 0.460 (−1.21, −4.45 to 2.02) | 0.529 (−1.03, −4.23 to 2.17) |
| Week 2 Change from Baseline: N, Mean (SD) | 114, −8.8 (21.60) | 19, −11.8 (16.39) | 50, −8.7 (11.11) |
| Week 2 Change from Baseline: LSMean (SE) | −9.1 (1.48) | −11.6 (1.85) | −8.4 (1.80) |
| TRT Diff vs. PBO: p-value (Diff, 95% CI) | | 0.161 (−3.96, −6.18 to 1.03) | 0.738 (0.60, −2.95 to 4.16) |
| Week 3 Change from Baseline: N, Mean (SD) | 140, −11.5 (14.10) | 81, −15.2 (17.45) | 68, −10.9 (13.17) |
| Week 3 Change from Baseline: LSMean (SE) | −11.6 (1.37) | −14.7 (2.02) | −10.9 (1.96) |
| TRT Diff vs. PBO: p-value (Diff, 95% CI) | | 0.134 (−3.11, −7.18 to 0.98) | 0.728 (0.71, −3.29 to 4.70) |
| Week 6 Change from Baseline: N, Mean (SD) | 137, −13.7 (15.06) | 59, −17.2 (17.53) | 64, −13.2 (13.66) |
| week 6 Change from Baseline: LSMean (SE) | −23.7 (1.59) | −17.1 (2.06) | −13.3 (2.00) |
| TRT Diff vs. PBO: p-value (Diff, 95% CI) | | 0.114 (−3.38, −7.58 to 0.32) | 0.843 (0.41, −3.70 to 4.53) |
| Stage 2 (Stage 1 Placebo Non-Responder Only) | | | |
| Baseline: N, Mean (SD) | 29, 69.9 (26.51) | 30, 70.2 (22.12) | 35, 67.0 (20.11) |
| Week 9 Change from Baseline: N, Mean (SD) | 29, 0.3 (7.34) | 30, −6.0 (10.19) | 35, −3.1 (10.79) |
| Week 9 Change from Baseline: LSMean (SE) | 0.4 (1.69) | −5.8 (1.66) | −3.3 (1.54) |
| TRT Diff vs. PBO: p-value (Diff, 95% CI) | | 0.010 (−6.24, −10.94 to −1.53) | 0.103 (−3.77, −8.31 to 0.79) |
| Week 12 Change from Baseline: N, Mean (SD) | 29, −1.1 (10.41) | 30, −5.5 (13.50) | 34, −5.5 (12.69) |
| Week 12 Change from Baseline: LSMean (SE) | −1.0 (2.19) | −5.3 (2.16) | 5.8 (2.02) |
| TRT Diff vs. PBO: p-value (Diff, 95% CI) | | 0.163 (−4.32, −10.43 to 1.79) | 0.106 (−4.87, −10.80 to 1.06) |
| OLS Z-statistic, p-value | | −2.116, p = 0.034 | −0.982, p = 0.326 |

TABLE 26LL
CMAI: Change from Baseline Parallel Group MMPM Analysis - Observed Data By CMAI Factor 1 Agitation Status
mITT 12-Week Parallel Group Population and CMAI Factor 1 Agitated at Baseline
Agressive Behavior (Factor 1): Agitated

| Parameter/Result | Placebo | AVP-786 18 mg | AVP-786 28 mg |
|---|---|---|---|
| Baseline: N, Mean (SD) | 46, 76.0 (22.81) | 64, 80.1 (22.46) | 66, 79.4 (23.78) |
| Week 1 Change from Baseline: N, Mean (SD) | 44, −8.0 (9.54) | 62, −8.2 (13.81) | 64, −8.2 (10.35) |
| Week 1 Change from Baseline: LSMean (SE) | −7.7 (2.07) | −8.1 (1.89) | −7.9 (1.84) |
| TRT Diff vs. PBO: p-value (Diff, 95% CI) | | 0.883 (−0.33, −4.73 to 4.07) | 0.925 (−0.21, −4.58 to 4.16) |
| Week 2 Change from Baseline: N, Mean (SD) | 38, −7.8 (12.48) | 49, −11.8 (16.39) | 50, −8.7 (11.11) |
| Week 2 Change from Baseline: LSMean (SE) | −8.7 (2.21) | −11.5 (2.02) | −8.2 (1.97) |
| TRT Diff vs. PBO: p-value (Diff, 95% CI) | | 0.267 (−2.75, −7.61 to 2.12) | 0.843 (0.49, −4.34 to 5.31) |
| Week 3 Change from Baseline: N, Mean (SD) | 45, −10.4 (16.22) | 61, −15.2 (17.45) | 66, −10.9 (13.17) |
| Week 3 Change from Baseline: LSMean (SE) | −10.6 (2.45) | −14.4 (2.20) | −10.7 (2.14) |
| TRT Diff vs. PBO: p-value (Diff, 95% CI) | | 0.189 (−3.72, −9.30 to 1.84) | 0.994 (−0.02, −5.53 to 5.49) |
| Week 6 Change from Baseline: N, Mean (SD) | 44, −12.3 (15.65) | 59, −17.2 (17.53) | 64, −13.2 (13.66) |
| week 6 Change from Baseline: LSMean (SE) | −12.4 (2.46) | −16.8 (2.21) | −13.1 (2.14) |
| TRT Diff vs. PBO: p-value (Diff, 95% CI) | | 0.127 (−4.36, −9.97 to 1.26) | 0.792 (−0.74, −6.28 to 4.80) |
| Week 9 Change from Baseline: N, Mean (SD) | 42, −12.0 (16.82) | 58, −18.5 (19.24) | 62, −13.0 (15.35) |
| Week 9 Change from Baseline: LSMean (SE) | −12.6 (2.62) | −18.2 (2.34) | −12.9 (2.26) |
| TRT Diff vs. PBO: p-value (Diff, 95% CI) | | 0.069 (−5.62, −11.70 to 0.45) | 0.915 (−0.32, −6.52 to 5.67) |
| Week 12 Change from Baseline: N, Mean (SD) | 43, −12.8 (17.78) | 59, −19.1 (18.62) | 64, −13.8 (14.74) |
| Week 12 Change from Baseline: LSMean (SE) | −13.2 (2.63) | −18.9 (2.34) | −13.9 (2.26) |
| TRT Diff vs. PBO: p-value (Diff, 95% CI) | | 0.067 (−5.71, −11.81 to 0.40) | 0.814 (−0.72, −6.73 to 5.29) |

TABLE 26MM
CMAI Total Score: Change from Baseline SPCD MMPM, Observed Data By Baseline MMSE (6-12, 13-16, 20-26)
mITT Population and CMAI Factor 1 Agitated at Baseline
6 <= Baseline MMSE <= 12

| Stage/ | | | |
|---|---|---|---|
| Parameter/Result | Placebo | AVP-786 18 mg | AVP-786 28 mg |
| Stage 1 | | | |
| Baseline: N, Mean (SD) | 52, 85.4 (27.66) | 21, 85.8 (24.30) | 22, 87.2 (22.64) |
| Week 1 Change from Baseline: N, Mean (SD) | 51, −6.4 (11.19) | 21, −14.1 (16.20) | 21, −8.5 (10.52) |
| Week 1 Change from Baseline: LSMean (SE) | −2.1 (3.76) | −10.9 (4.10) | −3.7 (4.12) |
| TRT Diff vs. PBO: p-value (Diff, 95% CI) | | 0.00 [symbol] (−0.74, −14.90 to −2.50) | 0.623 (−1.53, −7.67 to 4.62) |
| Week 2 Change from Baseline: N, Mean (SD) | 38, −7.3 (12.47) | 13, −16.5 (20.62) | 13, −9.5 (11.09) |
| Week 2 Change from Baseline: LSMean (SE) | −4.0 (3.84) | −13.4 (4.35) | −4.3 (4.35) |
| TRT Diff vs. PBO: p-value (Diff, 95% CI) | | 0.009 (−9.43, −16.46 to −2.40) | 0.7 [symbol] (−0.95, −7.92 to [symbol] .02) |
| Week 3 Change from Baseline: N, Mean (SD) | 49, −8.5 (15.66) | 29, −20.1 (18.61) | 22, −10.5 (14.20) |
| Week 3 Change from Baseline: LSMean (SE) | −4.4 (3.97) | −16.1 (4.60) | −5.7 (4.54) |
| TRT Diff vs. PBO: p-value (Diff, 95% CI) | | 0.004 (−11.68, −19.54 to −3.81) | 0.722 (−1.37, −9.01 to 6.27) |
| Week 6 Change from Baseline: N, Mean (SD) | 48, −11.3 (16.36) | 19, −22.0 (20.28) | 21, −11.2 (12.37) |
| week 6 Change from Baseline: LSMean (SE) | −7.0 (4.04) | −18.2 (4.78) | −8.7 (4.6 [symbol] ) |
| TRT Diff vs. PBO: p-value (Diff, 95% CI) | | 0.020 (−11.13, −29.54 to −2.73) | 0.940 (0.31, −7.79 to 3.41) |
| Stage 2 (Stage 1 Placebo Non-Responder Only) | | | |
| Baseline: N, Mean (SD) | 10, 76.4 (37.81) | 15, 81.1 (24.46) | 13, 72.5 (25.99) |
| Week 9 Change from Baseline: N, Mean (SD) | 10, 0.2 (8.11) | 15, −6.9 (11.70) | 13, −2.4 (15.44) |
| Week 9 Change from Baseline: LSMean (SE) | 0.2 ( [symbol] .77) | −6.3 (3.20) | −2.9 (3.32) |
| TRT Diff vs. PBO: p-value (Diff, 95% CI) | | 0.197 (− [symbol] .43, −16.35 to [symbol] .49) | 0.540 (−3.11, −13.38 to 7.10) |
| Week 12 Change from Baseline: N, Mean (SD) | 10, −0.1 (11.71) | 15, −5.5 (15.07) | 12, −2.8 (17.06) |
| Week 12 Change from Baseline: LSMean (SE) | −0.1 (4.48) | −4.7 (3.67) | −3.9 (4.07) |
| TRT Diff vs. PBO: p-value (Diff, 95% CI) | | 0.435 (−4.58, −16.36 to 7.20) | 0.543 (−3.72, −16.02 to 3.5 [symbol] ) |
| OLS Z-statistic, p-value | | −2.477, p = 0.013 | −0.37 [symbol] , p = 0.705 |

[symbol]

indicates data missing or illegible when filed

TABLE 26NN
CMAI Total Score: Change from Baseline SPCD MMPM, Observed Data By Baseline MMSE (6-12, 13-16, 20-26)
mITT Population and CMAI Factor 1 Agitated at Baseline
13 <= Baseline MMSE <= 19

| Stage/ | | | |
|---|---|---|---|
| Parameter/Result | Placebo | AVP-786 18 mg | AVP-786 28 mg |
| Stage 1 | | | |
| Baseline: N, Mean (SD) | 35, 76.3 (20.06) | 18, 81.0 (20.12) | 20, 77.7 (23.72) |
| Week 1 Change from Baseline: N, Mean (SD) | 34, −9.1 (10.26) | 18, −9.4 (14.02) | 19, −7.8 (7.30) |
| Week 1 Change from Baseline: LSMean (SE) | −12.9 (2.47) | −12.3 (2.90) | −20.7 (2.71) |
| TRT Diff vs. PBO: p-value (Diff, 95% CI) | | 0.813 (0.71, −5.27 to [symbol] .70) | 0.476 (2.12, −3.79 to 8.03) |
| Week 2 Change from Baseline: N, Mean (SD) | 31, −11.0 (13.04) | 15, −16.8 (17.76) | 16, −6.3 (8.58) |
| Week 2 Change from Baseline: LSMean (SE) | −15.0 (2.60) | −17.0 (3.14) | −3.9 (2.92) |
| TRT Diff vs. PBO: p-value (Diff, 95% CI) | | 0.533 (−2.09, −8.72 to 4.55) | 0.066 (4.05, −0.41 to 12.52) |
| Week 3 Change from Baseline: N, Mean (SD) | 35, −13.2 (12.79) | 18, −18.1 (20.11) | 20, −9.7 (10.39) |
| Week 3 Change from Baseline: LSMean (SE) | −17.5 (2.6 [symbol] ) | −20.1 (3.25) | −12.8 (3.02) |
| TRT Diff vs. PBO: p-value (Diff, 95% CI) | | 0.453 (−2.64, −9.63 to 4.34) | 0.176 (4.65, −2.19 to 11.43) |
| Week 6 Change from Baseline: N, Mean (SD) | 34, −15.2 (17.87) | 18, −19.9 (17.49) | 20, −11.0 (10. [symbol] ) |
| week 6 Change from Baseline: LSMean (SE) | −19.3 (2.89) | −21.9 (3.55) | −14.2 (3.31) |
| TRT Diff vs. PBO: p-value (Diff, 95% CI) | | 0.417 (−2.56, −10.39 to 5.28) | 0.178 (5.18, −2.41 to 12.78) |
| Stage 2 (Stage 1 Placebo Non-Responder Only) | | | |
| Baseline: N, Mean (SD) | 7, 66.6 (2.39) | 6, 63.3 (15.28) | 8, 64.3 (16.51) |
| Week 9 Change from Baseline: N, Mean (SD) | 7, 2.4 (5.97) | [symbol] , −8.8 (10.42) | 8, −2.0 (9.97) |
| Week 9 Change from Baseline: LSMean (SE) | 2.7 (3.35) | −9.0 (3.81) | −2.1 (3.13) |
| TRT Diff vs. PBO: p-value (Diff, 95% CI) | | 0.029 (−11.73, −22.12 to −1.32) | 0.314 (−4.76, −14.42 to 4.91) |
| Week 12 Change from Baseline: N, Mean (SD) | 7, 1.1 (3.09) | 6, −9.8 (11.74) | 8, −6.1 (9.75) |
| Week 12 Change from Baseline: LSMean (SE) | 1.3 (3.77) | −10.0 (4.07) | −6.2 (3.52) |
| TRT Diff vs. PBO: p-value (Diff, 95% CI) | | 0.058 (−11.29, −23.02 to 0.43) | 0.165 (−7.50, −18.39 to 3.40) |
| OLS Z-statistic, p-value | | −1. [symbol] 63, p = 0.063 | 0.036, p = 0.971 |

[symbol]

indicates data missing or illegible when filed

TABLE 26OO
CMAI Total Score: Change from Baseline SPCD MMPM, Observed Data By Baseline MMSE (6-12, 13-16, 20-26)
mITT Population and CMAI Factor 1 Agitated at Baseline
20 <= Baseline MMSE <= 26

| Stage/ | | | |
|---|---|---|---|
| Parameter/Result | Placebo | AVP-786 18 mg | AVP-786 28 mg |
| Stage 1 | | | |
| Baseline: N, Mean (SD) | 56, 73.6 (17.05) | 25, 74.6 (22.02) | 24, 73.8 (23.91) |
| Week 1 Change from Baseline: N, Mean (SD) | 54, −6.5 (8.79) | 23, −1.9 (7.91) | 24, −8.2 (22.21) |
| Week 1 Change from Baseline: LSMean (SE) | −4.5 (1.82) | −0.3 (2.56) | −3.9 (2.47) |
| TRT Diff vs. PBO: p-value (Diff, 95% CI) | | 0.076 (4.13, −0.45 to 8.82) | 0.565 (−1.36, −6.04 to 3.32) |
| Week 2 Change from Baseline: N, Mean (SD) | 45, −8.5 (9. [symbol] 3) | 21, −5.3 (9.62) | 21, −10.0 (12.90) |
| Week 2 Change from Baseline: LSMean (SE) | − [symbol] . [symbol] (1.91) | −3.2 (3.52) | −7.4 (2.64) |
| TRT Diff vs. PBO: p-value (Diff, 95% CI) | | 0.164 (3.61, −1.50 to 8.72) | 0.8 [symbol] 6 (−0.55, −5.75 to 4.66) |
| Week 3 Change from Baseline: N, Mean (SD) | 86, −13.0 (13.23) | 24, −9.2 (12.70) | 24, −12.3 (14.60) |
| Week 3 Change from Baseline: LSMean (SE) | −11.3 (2.14) | −6.8 (2.92) | −10.1 (3.03) |
| TRT Diff vs. PBO: p-value (Diff, 95% CI) | | 0.149 (4.52, −1.65 to 10.63) | 0.706 (1.19, −5.07 to 7.46) |
| Week 6 Change from Baseline: N, Mean (SD) | 55, −14.9 (11.61) | 23, −11.36 (13.96) | 23, −26.9 (16.58) |
| week 6 Change from Baseline: LSMean (SE) | −13.1 (2.02) | −9.8 (2.74) | −14.8 (2.85) |
| TRT Diff vs. PBO: p-value (Diff, 95% CI) | | 0.256 (3.27, −2.41 to 8.94) | 0.556 (−1.72, −7.43 to 4.04) |
| Stage 2 (Stage 1 Placebo Non-Responder Only) | | | |
| Baseline: N, Mean (SD) | 12, 66.5 (16.62) | 9, 56.6 (10.55) | 14, 63.4 (25.47) |
| Week 9 Change from Baseline: N, Mean (SD) | 12, −0.9 (7.72) | 9, −2.7 (7.00) | 14, −4.3 (5.24) |
| Week 9 Change from Baseline: LSMean (SE) | 0.1 (1.60) | −4.3 (1.87) | −4.1 (1.47) |
| TRT Diff vs. PBO: p-value (Diff, 95% CI) | | 0.095 (−4.32, −9.43 to 0.79) | 0.064 (−4.16, − [symbol] .5 [symbol] to 0.26) |
| Week 12 Change from Baseline: N, Mean (SD) | 12, −3.3 (10.97) | 9, −2.6 (12.40) | 14, −7.4 (10.05) |
| Week 12 Change from Baseline: LSMean (SE) | −2.4 (3.05) | −4.1 (3.56) | −4.2 (2.79) |
| TRT Diff vs. PBO: p-value (Diff, 95% CI) | | 0.711 (−1.79, −11.50 to 7.94) | 0.252 (−4.81, −13.23 to 3.60) |
| OLS Z-statistic, p-value | | 0.486, p = 0.627 | −1.232, p = 0.218 |

[symbol]

indicates data missing or illegible when filed

TABLE 26PP
CMAI: Change from Baseline Parallel Group MMPM Analysis - Observed Data By Baseline MMSE (6-12, 13-19, 20-26)
mITT 12-Week Parallel Group Population and CMAI Factor 1 Agitated at Baseline
6 <= Baseline MMSE <= 12

| Parameter/Result | Placebo | AVP-786 18 mg | AVP-786 28 mg |
|---|---|---|---|
| Baseline: N, Mean (SD) | 15, [symbol] 1.7 (32.64) | 21, 85.8 (24.30) | 22, 87.2 (22.64) |
| Week 1 Change from Baseline: N, Mean (SD) | 14, −5.9 (8.61) | 21, −14.1 (18.20) | 21, −8.5 (10.52) |
| Week 1 Change from Baseline: LSMean (SE) | 2.7 (5.72) | −7.8 (5.03) | −0.3 (5.20) |
| TRT Diff vs. PBO: p-value (Diff, 95% CI) | | 0.020 (−10.24, −18.81 to −1.68 | 0.498 (−2.94, −11.58 to 5.70) |
| Week 2 Change from Baseline: N, Mean (SD) | 15, −6.2 (14.67) | 13, −16.5 (20.62) | 13, −9.5 (11.09) |
| Week 2 Change from Baseline: LSMean (SE) | 0.6 (5.96) | −11.5 (5.35) | −1.5 (5.51) |
| TRT Diff vs. PBO: p-value (Diff, 95% CI) | | 0.018 (−12.09, − [symbol] 2.06 to −2.1 [symbol] | 0.679 (−2.07, −12.07 to 7.92) |
| Week 3 Change from Baseline: N, Mean (SD) | 14, −6.9 (19.53) | 19, −10.1 (18.61) | 22, −10.5 (14.20) |
| Week 3 Change from Baseline: LSMean (SE) | 1.1 (6.36) | −12.5 (5.59) | −3.2 (5.68) |
| TRT Diff vs. PBO: p-value (Diff, 95% CI) | | 0.019 (−13.63, −24.96 to −2.30 | 0.557 (−3.30, −14.52 to 7.91) |
| Week 6 Change from Baseline: N, Mean (SD) | 14, −10.6 (19.22) | 13, −22.0 (20.33) | 21, −11.2 (12.37) |
| week 6 Change from Baseline: LSMean (SE) | −2.2 (6.50) | −14.8 (5.72) | −3.4 (5.79) |
| TRT Diff vs. PBO: p-value (Diff, 95% CI) | | 0.039 (−12.59, −12.51 to −0.68 | 0.848 (−1.16, −12.89 to 10. [symbol] 3 |
| Week 9 Change from Baseline: N, Mean (SD) | 14, −10.8 (21.23) | 18, −19.0 (23.96) | 20, −11.2 (15.05) |
| Week 9 Change from Baseline: LSMean (SE) | −2.9 (7.13) | −13.3 (6.24) | −3.6 (6.26) |
| TRT Diff vs. PBO: p-value (Diff, 95% CI) | | 0.149 (−10.37, −24. [symbol] 8 to 3.84) | 0.922 (−0.69, −14.70 to 13.32 |
| Week 12 Change from Baseline: N, Mean (SD) | 14, −10.9 (22.35) | 18, −17.2 (20.63) | 21, −10.4 (13.76) |
| Week 12 Change from Baseline: LSMean (SE) | −2.6 (6.80) | −11.1 (5.96) | −2.8 (6.01) |
| TRT Diff vs. PBO: p-value (Diff, 95% CI) | | 0.198 (−8.47, −21.53 to 4.53) | 0.978 (−0.18, −13.04 to 12.66 |

[symbol]

indicates data missing or illegible when filed

TABLE 26QQ
CMAI: Change from Baseline Parallel Group MMRM Analysis - Observed Data
By Baseline MMSE (6-12, 13-19, 20-26)
mITT 12-Week Parallel Group Population and CMAI Factor 1 Agitated at Baseline
13 <= Baseline MMSE <= 19

| Parameter/Result | Placebo | | AVP-786 18 mg | | AVP-786 28 mg | |
|---|---|---|---|---|---|---|
| Baseline: N, Mean (SD) | 12, 74.3 | (18.24) | 18, 81.0 | (20.12) | 20, 77.7 | (23.72) |
| Week 1 Change from Baseline: N, Mean (SD) | 11, −8.5 | (10.82) | 18, −9.4 | (14.01) | 19, −7.8 | (7.80) |
| Week 1 Change from Baseline: LSMean (SE) | −12.3 | (3.50) | −13.4 | (3.13) | −11.9 | (2.90) |
| TRT Diff vs. PBO: p-value (Diff, 95% CI) | | | 0.791 (−1.11, −9.46 to 7.24) | | 0.935 (0.33, −7.85 to 9.51) | |
| Week 2 Change from Baseline: N, Mean (SD) | 10, −8.2 | (9.95) | 15, −16.8 | (17.76) | 16, −6.3 | (3.58) |
| Week 2 Change from Baseline: LSMean (SE) | −13.0 | (3.62) | −18.5 | (3.27) | −10.0 | (3.02) |
| TRT Diff vs. PBO: p-value (Diff, 95% CI) | | | 0.202 (−5.58, −14.26 to 3.10) | | 0.489 (2.93, −5.53 to 11.39 | |
| Week 3 Change from Baseline: N, Mean (SD) | 12, −10.8 | (10.84) | 18, −18.1 | (10.11) | 20, −9.7 | (10.39) |
| Week 3 Change from Baseline: LSMean (SE) | −15.5 | (3.95) | −21.4 | (3.51) | −14.0 | (3.24) |
| TRT Diff vs. PBO: p-value (Diff, 95% CI) | | | 0.228 (−5.85, −15.49 to 3.79) | | 0.733 (1.60, −7.78 to 10.97 | |
| Week 6 Change from Baseline: N, Mean (SD) | 11, −11.0 | (17.06) | 18, −19.9 | (17.49) | 20, −11.0 | (10.66) |
| Week 6 Change from Baseline: LSMean (SE) | −15.1 | (4.10) | −23.3 | (3.55) | −15.2 | (3.28) |
| TRT Diff vs. PBO: p-value (Diff, 95% CI) | | | 0.105 (−8.20, −18.16 to 1.79) | | 0.983 (−0.10, −9.82 to 9.62) | |
| Week 9 Change from Baseline: N, Mean (SD) | 11, −9.0 | (14.96) | 18, −23.3 | (17.02) | 20, −12.5 | (10.73) |
| Week 9 Change from Baseline: LSMean (SE) | −13.0 | (4.03) | −26.8 | (3.52) | −16.8 | (3.25) |
| TRT Diff vs. PBO: p-value (Diff, 95% CI) | | | 0.007 (−13.72, −23.55 to −3.90 | | 0.435 (−3.73, −13.29 to 5.83) | |
| Week 12 Change from Baseline: N, Mean (SD) | 11, −9.0 | (16.83) | 18, −22.6 | (20.54) | 20, −14.7 | (15.64) |
| Week 12 Change from Baseline: LSMean (SE) | −13.5 | (4.85) | −25.7 | (4.11) | −19.1 | (3.82) |

| | | |
|---|---|---|
| TRT Diff vs. PBO: p-value (Diff, 95% CI) | 0.047 (−12.20, −24.22 to −0.18 | 0.342 (−5.58, −17.29 to 6.13) |

TABLE 26RR
CMAI: Change from Baseline Parallel Group MMRM Analysis - Observed Data
By Baseline MMSE (6-12, 13-19, 20-26)
mITT 12-Week Parallel Group Population and CMAI Factor 1 Agitated at Baseline
20 <= Baseline MMSE <= 26

| Parameter/Result | Placebo | | AVP-786 18 mg | | AVP-786 28 mg | |
|---|---|---|---|---|---|---|
| Baseline: N, Mean (SD) | 19, 72.5 | (14.33) | 25, 74.6 | (22.02) | 24, 73.8 | (23.91) |
| Week 1 Change from Baseline: N, Mean (SD) | 19, −9.3 | (9.68) | 23, −1.9 | (7.91) | 24, −8.2 | (12.21) |
| Week 1 Change from Baseline: LSMean (SE) | −7.6 | (2.86) | −0.4 | (2.70) | −6.1 | (2.83) |
| TRT Diff vs. PBO: p-value (Diff, 95% CI) | | | 0.028 (7.18, 0.81 to 13.56 | | 0.639 (1.52, −4.95 to 8.00) | |
| Week 2 Change from Baseline: N, Mean (SD) | 13, −9.5 | (12.20) | 21, −5.3 | (9.62) | 21, −10.0 | (12.90) |
| Week 2 Change from Baseline: LSMean (SE) | −8.1 | (3.23) | −3.0 | (2.91) | −7.5 | (3.05) |
| TRT Diff vs. PBO: p-value (Diff, 95% CI) | | | 0.171 (5.09, −2.27 to 12.45 | | 0.862 (0.65, −6.83 to 8.14) | |
| Week 3 Change from Baseline: N, Mean (SD) | 19, 12.8 | (16.73) | 24, −9.2 | (12.70) | 24, −12.3 | (14.60) |
| Week 3 Change from Baseline: LSMean (SE) | −11.4 | (3.61) | −8.7 | (3.30) | −10.2 | (3.44) |
| TRT Diff vs. PBO: p-value (Diff, 95% CI) | | | 0.285 (4.65, −3.96 to 13.26 | | 0.791 (1.16, −7.57 to 9.90) | |
| Week 6 Change from Baseline: N, Mean (SD) | 19, −14.2 | (12.24) | 23, −11.3 | (13.93) | 23, −16.9 | (18.58) |
| Week 6 Change from Baseline: LSMean (SE) | −12.9 | (3.40) | −9.7 | (3.15) | −15.0 | (3.28) |
| TRT Diff vs. PBO: p-value (Diff, 95% CI) | | | 0.431 (3.18, −4.84 to 11.20 | | 0.611 (−2.08, −10.22 to 6.06) | |
| Week 9 Change from Baseline: N, Mean (SD) | 17, −15.0 | (14.20) | 22, −14.0 | (13.57) | 22, −15.0 | (19.17) |
| Week 9 Change from Baseline: LSMean (SE) | −14.4 | (3.53) | −12.3 | (3.24) | −12.9 | (3.39) |
| TRT Diff vs. PBO: p-value (Diff, 95% CI) | | | 0.617 (2.11, −6.28 to 10.51 | | 0.722 (1.52, −6.99 to 10.04 | |
| Week 12 Change from Baseline: N, Mean (SD) | 18, −16.7 | (14.27) | 23, −17.9 | (15.65) | 23, −16.0 | (14.88) |
| Week 12 Change from Baseline: LSMean (SE) | −15.4 | (3.44) | −15.9 | (3.18) | −14.9 | (3.31) |

| | | |
|---|---|---|
| TRT Diff vs. PBO: p-value (Diff, 95% CI) | 0.915 (−0.44, −9.59 to 7.72) | 0.994 (0.55, −7.70 to 8.81) |

TABLE 26SS
CMAI Total Score: Change from Baseline SPCD MMRM, Observed Data
By Age Group (<65, >=65)
mITT Population and CMAI Factor 1 Agitated at Baseline
Age <65 Years

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 10, 71.8 | (13.32) | 7, 79.0 | (19.13) | 5, 69.9 | (10.87) |
| Week 1 Change from Baseline: N, Mean (SD) | 9, −5.1 | (7.24) | 7, −9.3 | (7.34) | 5, −10.2 | (8.76) |
| Week 1 Change from Baseline: LSMean (SE) | −3.6 | (2.72) | −12.4 | (3.65) | −13.4 | (4.09) |
| TRT Diff vs. PBO: p-value (Diff, 95% CI) | | | 0.078 (−8.79, −18.70 to 1.11) | | 0.056 (−9.07, −20.03 to 0.39) | |
| Week 2 Change from Baseline: N, Mean (SD) | 9, −2.9 | (10.15) | 5, −16.8 | (5.40) | 3, −10.0 | (10.15) |
| Week 2 Change from Baseline: LSMean (SE) | −3.7 | (2.46) | −21.8 | (3.59) | −13.4 | (4.15) |
| TRT Diff vs. PBO: p-value (Diff, 95% CI) | | | −0.001 (−18.10, −27.59 to −8.62 | | 0.056 (−9.68, −19.68 to 0.26) | |
| Week 3 Change from Baseline: N, Mean (SD) | 9, −7.9 | (12.82) | 7, −17.6 | (7.44) | 5, −13.8 | (7.98) |
| Week 3 Change from Baseline: LSMean (SE) | −6.4 | (3.29) | −21.2 | (4.16) | −16.6 | (4.70) |
| TRT Diff vs. PBO: p-value (Diff, 95% CI) | | | 0.015 (−14.86, −26.40 to −3.31) | | 0.087 (−10.26, −22.19 to 1.67) | |
| Week 6 Change from Baseline: N, Mean (SD) | 9, −2.9 | (17.03) | 7, −13.1 | (2.85) | 4, −15.5 | (10.35) |
| Week 6 Change from Baseline: LSMean (SE) | −1.2 | (4.06) | −17.0 | (4.97) | −18.7 | (6.05) |
| TRT Diff vs. PBO: p-value (Diff, 95% CI) | | | 0.028 (−15.82, −29.67 to −1.97) | | 0.026 (−17.48, −32.61 to −2.35) | |
| Stage 2 (Stage 1 Placebo Non-Responder Only) | | | | | | |
| Baseline: N, Mean (SD) | 2, 101.0 | (2.83) | 1, 58.0 | ( ) | 3, 64.7 | (19.55) |
| Week 9 Change from Baseline: N, Mean (SD) | 2, −1.0 | (2.83) | 1, −11.0 | ( ) | 3, −3.0 | (10.32) |
| Week 9 Change from Baseline: LSMean (SE) | 12.4 | (4.03) | −20.4 | (4.14) | −9.3 | (2.45) |
| TRT Diff vs. PBO: p-value (Diff, 95% CI) | | | 0.041 (−32.82, −62.49 to −3.14) | | 0.062 (−21.28, −45.26 to 2.70) | |
| Week 12 Change from Baseline: N, Mean (SD) | 2, −17.5 | (13.44) | 1, −19.0 | ( ) | 3, −8.3 | (12.50) |
| Week 12 Change from Baseline: LSMean (SE) | −4.5 | (13.89) | −28.1 | (14.26) | −14.0 | (8.43) |

| | | |
|---|---|---|
| TRT Diff vs. PBO: p-value (Diff, 95% CI) | 0.426 (−23.56, −125.3 to 78.65) | 0.671 (−9.47, −92.05 to 73.11) |
| OLS Z-statistic, p-value | −1.839, p = 0.066 | −1.621, p = 0.105 |

TABLE 26TT
CMAI Total Score: Change from Baseline SPCD MMRM, Observed Data
By Age Group (<65, >=65)
mITT Population and CMAI Factor 1 Agitated at Baseline
Age >=65 Years

| Stage/ | | | | | | |
|---|---|---|---|---|---|---|
| Parameter/Result | Placebo | | AVP-786 18 mg | | AVP-786 28 mg | |
| Stage 1 | | | | | | |
| Baseline: N, Mean (SD) | 133, 79.0 | (23.12) | 57, 80.2 | (22.99) | 61, 80.2 | (24.42) |
| Week 1 Change from Baseline: N, Mean (SD) | 130, −7.2 | (10.22) | 55, −8.1 | (14.47) | 59, −8.0 | (10.52) |
| Week 1 Change from Baseline: LSMean (SE) | −7.3 | (1.46) | −8.3 | (1.80) | −7.9 | (1.75) |
| TRT Diff vs. PBO: p-value (Diff, 95% CI) | | | 0.607 (−0.91, −4.38 to 2.56) | | 0.743 (−0.56, −3.95 to 2.92) | |
| Week 2 Change from Baseline: N, Mean (SD) | 105, −9.3 | (11.62) | 44, −11.2 | (17.15) | 47, −8.6 | (11.26) |
| Week 2 Change from Baseline: LSMean (SE) | −9.5 | (1.52) | −10.9 | (1.93) | −8.2 | (1.86) |
| TRT Diff vs. PBO: p-value (Diff, 95% CI) | | | 0.493 (−1.33, −5.14 to 2.48) | | 0.493 (1.30, −2.42 to 5.01) | |
| Week 3 Change from Baseline: N, Mean (SD) | 131, −11.7 | (14.19) | 54, −14.9 | (18.38) | 61, −10.6 | (13.52) |
| Week 3 Change from Baseline: LSMean (SE) | −12.1 | (1.62) | −14.5 | (2.11) | −10.6 | (2.03) |
| TRT Diff vs. PBO: p-value (Diff, 95% CI) | | | 0.264 (−2.46, −6.79 to 1.86) | | 0.502 (1.43, −2.76 to 5.62) | |
| Week 6 Change from Baseline: N, Mean (SD) | 128, −14.5 | (14.69) | 52, −17.8 | (18.60) | 60, −13.0 | (13.89) |
| Week 6 Change from Baseline: LSMean (SE) | −14.6 | (1.63) | −17.8 | (2.14) | −13.1 | (2.04) |
| TRT Diff vs. PBO: p-value (Diff, 95% CI) | | | 0.157 (−3.17, −7.57 to 1.23) | | 0.475 (1.54, −2.70 to 5.77) | |
| Stage 2 (Stage 1 Placebo Non-Responder Only) | | | | | | |
| Baseline: N, Mean (SD) | 27, 67.6 | (26.02) | 29, 70.6 | (22.39) | 32, 67.2 | (20.45) |
| Week 9 Change from Baseline: N, Mean (SD) | 27, 0.4 | (7.59) | 29, −5.3 | (10.32) | 32, −3.1 | (10.95) |
| Week 9 Change from Baseline: LSMean (SE) | 0.3 | (1.79) | −5.5 | (1.73) | −3.2 | (1.64) |
| TRT Diff vs. PBO: p-value (Diff, 95% CI) | | | 0.023 (−5.76, −10.70 to −0.82) | | 0.153 (−3.50, −5.32 to 1.32) | |
| Week 12 Change from Baseline: N, Mean (SD) | 27, 0.1 | (9.96) | 29, −5.0 | (13.49) | 31, −5.2 | (12.88) |
| Week 12 Change from Baseline: LSMean (SE) | −0.0 | (2.26) | −4.7 | (2.18) | −5.4 | (2.10) |

| | | |
|---|---|---|
| TRT Diff vs. PBO: p-value (Diff, 95% CI) | 0.139 (−4.69, −10.94 to 1.56) | 0.084 (−5.38, −11.52 to 0.75) |
| OLS Z-statistic, p-value | −1.057, p = 0.040 | −0.690, p = 0.490 |

TABLE 26UU
CMAI: Change from Baseline Parallel Group MMRM Analysis - Observed Data
By Age Group (<65, >=65)
mITT 12-Week Parallel Group Population and CMAI Factor 1 Agitated at Baseline
Age <65 Years

| Parameter/Result | Placebo | | AVP-786 18 mg | | AVP-786 28 mg | |
|---|---|---|---|---|---|---|
| Baseline: N, Mean (SD) | 3, 79.7 | (9.07) | 7, 79.0 | (19.13) | 5, 69.8 | (10.37) |
| Week 1 Change from Baseline: N, Mean (SD) | 2, 2.0 | (15.58) | 7, −9.3 | (7.34) | 5, −10.2 | (8.76) |
| Week 1 Change from Baseline: LSMean (SE) | ( ) | | ( ) | | ( ) | |
| Week 2 Change from Baseline: N, Mean (SD) | 3, 7.3 | (9.87) | 5, −16.8 | (5.40) | 3, −10.0 | (10.15) |
| Week 2 Change from Baseline: LSMean (SE) | ( ) | | ( ) | | ( ) | |
| Week 3 Change from Baseline: N, Mean (SD) | 2, 9.5 | (20.51) | 7, −17.6 | (7.44) | 5, −13.8 | (7.98) |
| Week 3 Change from Baseline: LSMean (SE) | ( ) | | ( ) | | ( ) | |
| Week 6 Change from Baseline: N, Mean (SD) | 2, 22.0 | (9.90) | 7, −13.1 | (2.85) | 4, −15.5 | (10.85) |
| Week 6 Change from Baseline: LSMean (SE) | ( ) | | ( ) | | ( ) | |
| Week 9 Change from Baseline: N, Mean (SD) | 2, 21.0 | (12.73) | 7, −11.1 | (25.00) | 5, −15.4 | (15.11) |
| Week 9 Change from Baseline: LSMean (SE) | ( ) | | ( ) | | ( ) | |
| Week 12 Change from Baseline: N, Mean (SD) | 2, 4.5 | (3.54) | 7, −10.4 | (13.18) | 5, −21.6 | (16.92) |

| | | | |
|---|---|---|---|
| Week 12 Change from Baseline: LSMean (SE) | ( ) | ( ) | ( ) |

TABLE 26VV
CMAI: Change from Baseline Parallel Group MMRM Analysis - Observed Data
By Age Group (<65, >=65)
mITT 12-Week Parallel Group Population and CMAI Factor 1 Agitated at Baseline
Age >=65 Years

| Parameter/Result | Placebo | | AVP-786 18 mg | | AVP-786 28 mg | |
|---|---|---|---|---|---|---|
| Baseline: N, Mean (SD) | 43, 75.7 | (23.30) | 57, 80.2 | (22.99) | 61, 80.2 | (24.42) |
| Week 1 Change from Baseline: N, Mean (SD) | 42, −8.5 | (9.19) | 55, −8.1 | (14.47) | 59, −8.0 | (10.52) |
| Week 1 Change from Baseline: LSMean (SE) | −8.7 | (2.12) | −7.9 | (1.95) | −7.6 | (1.89) |
| TRT Diff vs. PBO: p-value (Diff, 95% CI) | | | 0.734 (0.79, −3.82 to 5.41) | | 0.637 (1.09, −3.47 to 5.64) | |
| Week 2 Change from Baseline: N, Mean (SD) | 35, −9.1 | (11.91) | 44, −11.2 | (17.15) | 47, −8.6 | (11.26) |
| Week 2 Change from Baseline: LSMean (SE) | −9.8 | (2.27) | −10.4 | (2.08) | −7.8 | (2.01) |
| TRT Diff vs. PBO: p-value (Diff, 95% CI) | | | 0.316 (−0.60, −5.69 to 4.49) | | 0.423 (2.04, −2.98 to 7.05) | |
| Week 3 Change from Baseline: N, Mean (SD) | 43, −11.4 | (15.68) | 54, −14.9 | (18.38) | 61, −10.6 | (13.52) |
| Week 3 Change from Baseline: LSMean (SE) | −12.1 | (2.52) | −13.9 | (2.29) | −10.1 | (2.20) |
| TRT Diff vs. PBO: p-value (Diff, 95% CI) | | | 0.547 (−1.78, −7.62 to 4.05) | | 0.492 (2.00, −3.73 to 7.72) | |
| Week 6 Change from Baseline: N, Mean (SD) | 42, −13.9 | (13.95) | 52, −17.3 | (13.60) | 60, −13.0 | (13.89) |
| Week 6 Change from Baseline: LSMean (SE) | −14.6 | (2.49) | −17.2 | (2.23) | −12.6 | (2.18) |
| TRT Diff vs. PBO: p-value (Diff, 95% CI) | | | 0.362 (−2.67, −8.45 to 3.10) | | 0.502 (1.92, −3.72 to 7.57) | |
| Week 9 Change from Baseline: N, Mean (SD) | 40, −13.7 | (15.31) | 51, −19.5 | (18.40) | 57, −12.8 | (15.49) |
| Week 9 Change from Baseline: LSMean (SE) | −14.8 | (2.51) | −19.2 | (2.28) | −12.5 | (2.19) |
| TRT Diff vs. PBO: p-value (Diff, 95% CI) | | | 0.135 (−4.42, −10.22 to 1.39) | | 0.426 (2.30, −3.39 to 7.98) | |
| Week 12 Change from Baseline: N, Mean (SD) | 41, −13.7 | (17.78) | 52, −20.3 | (19.03) | 59, −13.1 | (14.51) |
| Week 12 Change from Baseline: LSMean (SE) | −14.4 | (2.66) | −20.1 | (2.42) | −13.0 | (2.31) |

| | | |
|---|---|---|
| TRT Diff vs. PBO: p-value (Diff, 95% CI) | 0.078 (−5.64, −11.91 to 0.63) | 0.654 (1.39, −4.73 to 7.52) |

### 5.3.1.3. Additional CMAI Analyses

The CMAI Total score was also analyzed for the percentage of patients meeting response criteria, with response criteria defined as a 30% or 50% improvement in the CMAI Total score compared to Baseline. There were no significant between-group differences in response rate using either response criteria in the overall SPCD analysis, in either stage individually, or in the 12-week Parallel Group analysis.

Agitated Status was defined as the presence of any 1 CMAI factor (F1—Aggressive Behavior, F2—Physically Nonaggressive Behavior, or F3-Verbally Agitated Behavior) scored as “agitated.” At the end of Stage 1, a significantly lower percentage of patients treated with AVP-786-18 met criteria for agitated status compared with placebo (80.4% and 92.6%, respectively; p=0.0029). A similar percentage of patients treated with AVP-786-28 and placebo met criteria for agitated status (90.7% and 92.6%, respectively; p=0.2416). At the end of Stage 2, a lower percentage of patients treated with AVP-786-28 met criteria for agitated status compared with placebo (86.4% and 95.0%, respectively), which did not reach significance (p=0.6158). A similar percentage of patients treated with AVP-786-18 and placebo met criteria for agitated status (95.1% and 95.0%, respectively; p=1.000). The overall SPCD 1 degree of freedom p-values could not be calculated because of sparse cell count.

Agitated Status for the 12-week Parallel Group was calculated. At Week 12, a lower percentage of patients treated with AVP-786-18 and AVP-786-28 met criteria for agitated status compared with placebo (87.2%, 91.3%, and 94.7% respectively). The p-values could not be calculated because the general estimating equation model did not converge.

### 5.3.1.4. Secondary Efficacy Endpoints

5.3.1.4.1. mADCS-CGIC-Agitation Score

### 5.3.1.4.1.1. Stage 1 and Stage 2 (Placebo Nonresponders)

The key secondary efficacy endpoint was the SPCD analysis of the mADCS-CGIC-Agitation score for AVP-786-18 and AVP-786-28 versus placebo (Table 26).

In the SPCD analysis, patients treated with AVP-786-18 showed greater improvement in the mean mADCS-CGIC-Agitation score compared with placebo (significant at the nominal level, p=0.012). For patients treated with AVP-786-28 compared with placebo, the p-value was not significant at the nominal level (p=0.097).

In Stage 1, the treatment differences (CI) in the mean mADCS-CGIC-Agitation score for patients treated with AVP-786-18 and AVP-786-28 compared with placebo were not significantly different (p=0.331 and p=0.400, respectively).

In Stage 2, patients treated with AVP-786-18 showed greater improvement in the mean mADCS-CGIC-Agitation score compared with placebo (treatment difference [CI]: −0.6 [−1.1 to −0.1), which was significant at the nominal level (p=0.014). The treatment difference (CI) for patients treated with AVP-786-28 compared with placebo was −0.4 (−0.9 to 0.1), with a p=0.145.

TABLE 26
mADCS-CGIC-Agitation Score: SPCD ANCOVA-LOCF Data (mITT Population)

| Stage | | | AVP-786 | | AVP-786 | |
|---|---|---|---|---|---|---|
| Parameter/Results | Placebo | | 18 mg | | 28 mg | |
| Stage 1 | | | | | | |
| N | 184 | | 90 | | 96 | |
| 1 = Marked Improvement, n (%) | 6 | (3.3) | 9 | (10.0) | 5 | (5.2) |
| 2 = Moderate improvement, n (%) | 30 | (16.3) | 12 | (13.3) | 18 | (18.8) |
| 3 = Minimal Improvement, n (%) | 71 | (38.6) | 36 | (40.0) | 34 | (35.4) |
| 4 = No change, n (%) | 57 | (31.0) | 25 | (27.8) | 28 | (29.2) |
| 5 = Minimal Worsening, n (%) | 12 | (6.5) | 4 | (4.4) | 8 | (8.3) |
| 6 = Moderate Worsening, n (%) | 4 | (2.2) | 2 | (2.2) | 3 | (3.1) |
| 7 = Marked Worsening, n (%) | 4 | (2.2) | 2 | (2.2) | 0 | |
| Week 6 Score: Mean (SD) | 3.4 | (1.13) | 3.2 | (1.24) | 3.3 | (1.12) |
| Week 6 Score: LS Mean (SE) [1] | 3.4 | (0.12) | 3.2 | (0.15) | 3.2 | (0.15) |
| ANCOVA LS Mean Diff vs. Placebo: p-value | | | 0.331 | (−0.1, −0.4 to 0.1) | 0.400 | (−0.1, −0.4 to 0.2) |
| (Dif. 95% CI) [1] | | | | | | |
| Stage 2 (Placebo Nonresponders only) | 40 | | 40 | | 42 | |
| 1 = Marked Improvement, n (%) | 0 | | 1 | (2.5) | 2 | (4.8) |
| 2 = Moderate Improvement, n (%) | 3 | (7.5) | 6 | (15.0) | 7 | (16.7) |
| 3 = Minimal Improvement, n (%) | 12 | (30.0) | 17 | (42.5) | 13 | (31.0) |
| 4 = No change, n (%) | 14 | (35.0) | 13 | (32.5) | 12 | (28.6) |
| 5 = Minimal Worsening, n (%) | 8 | (20.0) | 3 | (7.5) | 4 | (9.5) |
| 6 = Moderate Worsening, n (%) | 3 | (7.5) | 0 | | 3 | (71) |
| 7 = Marked Worsening, n (%) | 0 | | 0 | | 1 | (2.4) |
| Week 12 Score Relative to Week 6: Mean (SD) | 3.9 | (1.06) | 3.3 | (0.91) | 3.5 | (1.35) |
| Week 12 Score Relative to Week 6: LS Mean (SE) [1] | 3.9 | (0.18) | 3.3 | (0.18) | 3.5 | (0.17) |
| ANCOVA LS Mean Diff vs. Placebo: p-value | | | 0.014 | (−0.6. −1.1 to −0.1) | 0.145 | (−0.4, −0.9 to 0.1) |
| (Dif. 95% CI) [1] | | | | | | |

| | | |
|---|---|---|
| SPCD OLS weighted z-statistic, p-value [2] | −2.51, p = 0.012 | −1.66, p = 0.097 |

Note:
mADCS-CGIC-Agitation scores range from 1 to 7 with lower scores indicating improvement.
[1] Treatment effects were estimated at each stage by ANCOVA with fixed effects for treatment, Baseline CMAI Total score, and in the Stage 1 model, Baseline NPI AA (≤6 vs >6), risk assessments for falls (normal/mild vs moderate/severe), Baseline concomitant use of antipsychotic medications (yes vs no). Stage 1 and 2 Baseline values were used in their respective models. Missing values were imputed by LOCF within each stage.
[2] OLS weighted z-statistic was used with weight = 0.6 for Stage 1 and 0.4 for Stage 2.

Response on the mADCS-CGIC-Agitation score was defined as marked or moderate improvement and was calculated. In the SPCD analysis, the p-values were not significant at the nominal level for either dose of AVP-786 (p=0.3679 and p=0.0797 for AVP-786-18 compared with placebo and AVP-786-28 compared with placebo, respectively). In Stage 1, a similar percentage of patients treated with placebo, AVP-786-18, and AVP-786-28 had marked or moderate improvement on the mADCS-CGIC-Agitation score (19.6%, 23.3%, and 24.0%, respectively), with p-values of 0.4705 and 0.3922 for AVP-786-18 compared with placebo and AVP-786-28 compared with placebo, respectively. In Stage 2, a higher percentage of patients treated with AVP-786-18 and AVP-786-28 had marked or moderate improvement on the mADCS-CGIC-Agitation score compared with placebo (17.5%, 21.4%, and 7.5%, respectively), which did not reach significance (p=0.3109 and p=0.1172, respectively).

### 5.3.1.4.1.2. 12-Week Parallel Group

The change from Baseline in the mean mADCS-CGIC-Agitation score at Week 12 is presented for the 12-week Parallel Group in Table 27. The treatment differences (CI) for patients treated with AVP-786-18 and AVP-786-28 compared with placebo were −0.3 (−0.7 to 0.1) and −0.3 (−0.7 to 0.1), respectively (p=0.172 and p=0.191, respectively).

Response on the mADCS-CGIC-Agitation score was calculated. A higher percentage of patients treated with AVP-786-18 and AVP-786-28 had marked or moderate improvement on the mADCS-CGIC-Agitation score compared with placebo (41.4%, 38.0%, and 32.8%, respectively); however, the difference did not reach significance (p=0.3660 and p=0.5090, respectively).

TABLE 27
mADCS-CGIC-Agitation Score: Parallel Group MMRM Analysis-Observed Data
(mITT 12-week Parallel Group Population)

| Parameter/Results | Placebo [1] | | AVP-786-18 [1] | | AVP-786-28 [1] | |
|---|---|---|---|---|---|---|
| Week 12 Score N | 58 | | 87 | | 92 | |
| 1 = Marked Improvement, n (%) | 7 | (12.1) | 11 | (12.6) | 9 | (9.8) |
| 2 = Moderate Improvement, n (%) | 12 | (20.7) | 25 | (28.7) | 26 | (28.3) |
| 3 = Minimal Improvement, n (%) | 17 | (29.3) | 22 | (25.3) | 31 | (33.7) |
| 4 = No change, n (%) | 10 | (17.2) | 21 | (24.1) | 15 | (16.3) |
| 5 = Minimal Worsening, n (%) | 7 | (12.1) | 8 | (9.2) | 10 | (10.9) |
| 6 = Moderate Worsening, n (%) | 5 | (8.6) | 0 | | 1 | (1.1) |
| 7 = Marked Worsening, n (%) | 0 | | 0 | | 0 | |
| Week 12 Score: Mean (SD) | 3.2 | (1.45) | 2.9 | (1.19) | 2.9 | (1.17) |
| Week 12 Change from Baseline: LS Mean (SE) [2] | 3.2 | (0.19) | 2.9 | (0.17) | 2.9 | (0.16) |
| Treat Diff vs. Placebo: p-value (Dif, 95% CI) [2] | | | 0.172 | (−0.3, −0.7 to 0.1) | 0.191 | (−0.3, −0.7 to 0.1) |

Note:
mADCS-CGIC-Agitation scores range from 1 to 7 with lower scores indicating improvement. Scores are relative to the original Baseline.
[1] Patients who are randomized to same treatment group in both stages.
[2] MMRM with fixed effects of treatment, visit, treatment-by -visit, Baseline CMAI Total score, Baseline CMAI Total score-by-visit. Baseline NPI AA (≤6 vs. >6), risk assessment for falls (normal/mild vs. moderate/severe), Baseline concomitant use of antipsychotic medications (yes vs. no). Unstructured variance-covariance was used.

### 5.3.1.4.2. NPI Domain and Total Scores

The NPI domains pre-identified as endpoints, Agitation/Aggression (domain score and Caregiver Distress score), Aberrant Motor Behavior (domain score), and Irritability/Lability (domain score) and the NPI Total score, are summarized in Table 28 for the SPCD analysis and in Table 29 for the 12-week Parallel Group, and these results are discussed individually below with further data shown in Tables 29a and 29b. Other NPI endpoints are addressed below.

TABLE 28
NPI Domain and Total Scores: Change from Baseline SPCD MMRM-
Observed Data (mITT Population)

| Stage | | | | | | |
|---|---|---|---|---|---|---|
| Parameter/Results | Placebo | | AVP-786-18 | | AVP-786-28 | |
| Agitation/Aggression | | | | | | |
| (Domain Score) | | | | | | |
| Stage 1 | | | | | | |
| Baseline: N, Mean (SD) | 191, 7.1 | (2.39) | 94, 6.5 | (1.94) | 97, 7.2 | (2.42) |
| Week 6 Change from Baseline: | −1.6 | (0.27) | −2.1 | (0.36) | −1.9 | (0.35) |
| LS Mean (SE) [1] | | | | | | |
| Treat Diff vs. Placebo: | | | 0.182 | (−0.5, −1.3 to 0.2) | 0.390 | (−0.3, −1.1 to 0.4) |
| p-value (Dif, 95% CI) [1] | | | | | | |
| Stage 2 | | | | | | |
| (Placebo Nonresponders only) | | | | | | |
| Stage 2 Baseline: | 40, 6.9 | (2.84) | 41, 6.3 | (2.71) | 44, 6.3 | (2.81) |
| N, Mean (SD) [2] | | | | | | |
| Week 12 Change from Baseline: | −1.4 | (0.45) | −0.9 | (0.43) | −1.0 | (0.43) |
| LS Mean (SE) [1] | | | | | | |
| Treat Diff vs. Placebo: | | | 0.462 | (0.5,−0.8 to 1.7) | 0.528 | (0.4, −0.8 to 1.6) |
| p-valuc (Dif, 95% CI) [1] | | | | | | |
| SPCD Week 6 & 12 MMRM | | | −0.39, | p = 0.695 | −0.12, | p = 0.904 |
| weighted z-statistic, p-value [3] | | | | | | |
| Agitation/Aggression | | | | | | |
| (Caregiver Distress Score) | | | | | | |
| Stage 1 | | | | | | |
| Baseline: N, Mean (SD) | 191, 3.2 | (1.04) | 94, 3.3 | (1.01) | 97, 3.1 | (1.04) |
| Week 6 Change from Baseline: | −0.5 | (0.11) | −0.8 | (0.15) | −0.7 | (0.15) |
| LS Mean(SE) [1] | | | | | | |
| Treat Diff vs. Placebo: | | | 0.070 | (−0.3, −0.6 to 0.0) | 0.224 | (−0.2, −0.5 to 0.1) |
| p-value (Dif, 95% CI) [1] | | | | | | |
| Stage 2 | | | | | | |
| (Placebo Nonresponders only) | | | | | | |
| Stage 2 Baseline: | 40, 3.2 | (1.03) | 41, 2.9 | (1.14) | 44, 3.3 | (1.14) |
| N, Mean (SD) [2] | | | | | | |
| Week 12 Change from Baseline: | −0.4 | (0.17) | −0.2 | (0.17) | −0.5 | (0.17) |
| LS Mean (SE) [1] | | | | | | |
| Treat Diff vs. Placebo: | | | 0.545 | (0.1. −0.3 to 0.6) | 0.686 | (−0.1 −0.6 to 0.4) |
| p-value (Dif, 95% CI) [1] | | | | | | |
| SPCD Week 6 & 12 MMRM | | | −0.87, | p = 0.386 | −1.15, | p = 0.250 |
| weighted z-statistic, p-value [3] | | | | | | |
| Aberrant Motor Behavior | | | | | | |
| (Domain Score) | | | | | | |
| Stage 1 | | | | | | |
| Baseline: N, Mean (SD) | 191, 5.1 | (3.94) | 94, 4.9 | (3.77) | 97, 5.1 | (4.07) |
| Week 6 Change from Baseline: | −1.0 | (0.31) | −1.6 | (0.40) | −0.6 | (0.39) |
| LS Mean (SE) [1] | | | | | | |
| Treat Diff vs. Placebo: | | | 0.146 | (−0.6, −1.4 to 0.2) | 0.399 | (0.3, −0.4 to 1.1) |
| p-value (Dif, 95% CI) [1] | | | | | | |
| Stage 2 | | | | | | |
| (Placebo Nonresponders only) | | | | | | |
| Stage 2 Baseline: | 40, 5.2 | (3.79) | 41, 4.0 | (3.87) | 44, 4.3 | (4.32) |
| N, Mean (SD) [2] | | | | | | |
| Week 12 Change from Baseline: | −0.2 | (0.47) | 0.5 | (0.45) | 1.0 | (0.45) |
| LS Mean (SE) [1] | | | | | | |
| Treat Diff vs. Placebo: | | | 0.283 | (0.7, −0.6 to 2.0) | 0.065 | (1.2, −0.1 to 2.5) |
| p-value (Dif, 95% CI) [1] | | | | | | |
| SPCD Week 6 & 12 MMRM | | | −0.22, | p = 0.829 | 1.94, | p = 0.052 |
| weighted z-statistic, p-value [3] | | | | | | |
| Irritability/Lability | | | | | | |
| (Domain Score) | | | | | | |
| Stage 1 | | | | | | |
| Baseline: N, Mean (SD) | 191, 5.1 | (3.33) | 94, 5.1 | (3.62) | 97, 5.2 | (3.39) |
| Week 6 Change from Baseline: | −1.3 | (0.30) | −2.1 | (0.40) | −1.6 | (0.39) |
| LS Mean (SE) [1] | | | | | | |
| Treat Diff vs. Placebo: | | | 0.061 | (−0.8, −1.6 to 0.0) | 0.400 | (−0.3, −1.1 to 0.5) |
| p-value (Dif, 95% CI) [1] | | | | | | |
| Stage 2 | | | | | | |
| (Placebo Nonresponders only) | | | | | | |
| Stage 2 Baseline: | 40, 4.5 | (3.99) | 41, 5.3 | (3.54) | 44, 5.2 | (3.64) |
| N, Mean (SD) [2] | | | | | | |
| Week 12 Change from Baseline: | −0.0 | (0.48) | −1.1 | (0.47) | −0.8 | (0.47) |
| LS Mean (SE) [1] | | | | | | |
| Treat Diff vs. Placebo: | | | 0.115 | (−1.1, −2.4 to 0.3) | 0.264 | (−0.8, −2.1 to 0.6) |
| p-value (Dif, 95% CI) [1] | | | | | | |
| SPCD Week 6 & 12 MMRM | | | −2.44, | p = 0.015 | −1.40, | p = 0.163 |
| weighted z-statistic, p-value [3] | | | | | | |
| NPI Total Score | | | | | | |
| Stage 1 | | | | | | |
| Baseline: N, Mean (SD) | 191, 40.2 | (17.95) | 94, 39.4 | (18.93) | 97, 40.1 | (19.98) |
| Week 6 Change from Baseline: | −9.9 | (1.46) | −13.8 | (1.90) | −11.4 | (1.85) |
| LS Mean (SE) [1] | | | | | | |
| Treat Diff vs. Placebo: | | | 0.050 | (−3.9, −7.8 to −0.0) | 0.412 | (−1.6, −5.4 to 2.2) |
| p-value (Dif, 95% CI) [1] | | | | | | |
| Stage 2 | | | | | | |
| (Placebo Nonresponders only) | | | | | | |
| Stage 2 Baseline: | 40, 39.2 | (22.47) | 41, 34.7 | (14.16) | 44, 34.6 | (16.93) |
| N, Mean (SD) [2] | | | | | | |
| Week 12 Change from Baseline: | −3.6 | (2.46) | −4.6 | (2.39) | −0.3 | (2.38) |
| LS Mean (SE) [1] | | | | | | |
| Treat Diff vs. Placebo: | | | 0.775 | (−1.0, −7.8 to 5.8) | 0.333 | (3.3, −3.5 to 10.1) |
| p-value (Dif, 95% CI) [1] | | | | | | |
| SPCD Week 6 & 12 MMRM | | | −1.50, | p = 0.133 | 0.22, | p = 0.829 |
| weighted z-statistic, p-value [3] | | | | | | |

CI = confidence interval;
Diff or Dif = difference;
LS = least squares;
mTT = modified intent-to-treat;
MMRM = mixed model repeated measures;
NPI AA = Neuropsychiatric Inventory Agitation/Aggtession Domain score;
SD = standard deviation;
SE = standard error;
SPCD = sequential parallel comparison design
Note:
Agitation/Aggression Domain-Caregiver Distress score ranges from 0 to 5.
Aberrant Motor Behavior Domain score ranges from 0 to 12.
Irritability/Lability Domain score ranges from 0 to 12.
NPI Total score ranges from 0 to 144.
For all scores, higher scores indicate greater worsening condition.
[1] MMRMs include fixed effects of treatment, visit, treatment-by-visit. Baseline, Baseline-by-visit, and in the Stage 1 model, Baseline NPI AA (≤6 vs >6), risk assessment for falls (normal/mild vs moderate/severe). Baseline concomitant use of antipsychotic medications (yes vs no). Unstructured variance-covariance was used. Stage 1 and 2 Baseline values were used in their respective models.
[2] Stage 2 Baseline is the last nonmissing assessment prior to Stage 2 rerandomization (rerandomization visit).
[3] SPCD Week 6 & 12 MMRM weighted z-statistic was used with weight = 0.6 for Slage 1 and 0.4 for Stage 2.

TABLE 29
NPI Domain and Total Scores: Change from Baseline 12-week Parallel
Group MMRM Analysis-Observed Data (mITT Population)

| Parameter/Results | Placebo [1] | | AVP-786-18 [1] | | AVP-786-28 [1] | |
|---|---|---|---|---|---|---|
| Agitation/Aggression | | | | | | |
| (Domain Score) | | | | | | |
| Baseline: N, Mean (SD) | 62, 7.2 | (2.52) | 94, 6.5 | (1.94) | 97, 7.2 | (2.42) |
| Week 12 Change from Baseline: | −2.2 | (0.42) | −2.7 | (0.36) | −2.8 | (0.36) |
| LS Mean (SE) [2] | | | | | | |
| Treat Diff vs. Placebo: p-valuc | | | 0.298 | (−0.5, −1.5 to 0.5) | 0.214 | (−0.6, −1.6 to 0.4) |
| (Dif, 95% CI) [2] | | | | | | |
| Agitation/Aggression | | | | | | |
| (Caregiver Distress Score) | | | | | | |
| Baseline: N, Mean (SD) | 62, 3.2 | (1.14) | 94, 3.3 | (1.01) | 97, 3.1 | (1.04) |
| Week 12 Change from Baseline: | −0.6 | (0.18) | −0.8 | (0.16) | −0.9 | (0.15) |
| LS Mean (SE) [2] | | | | | | |
| Treat Diff vs. Placebo: | | | 0.227 | (−0.3, −0.7 to 0.2) | 0.090 | (−0.4, −0.8 to 0.1) |
| p-value (Dif, 95% CI) [2] | | | | | | |
| Aberrant Motor Behavior | | | | | | |
| (Domain Score) | | | | | | |
| Baseline: N, Mean (SD) | 62, 4.8 | (3.74) | 94, 4.9 | (3.77) | 97, 5.1 | (4.07) |
| Week 12 Change from Baseline: | −0.8 | (0.46) | −1.3 | (0.41) | −0.7 | (0.40) |
| LS Mean (SE) [2] | | | | | | |
| Treat Diff vs. Placebo: | | | 0.307 | (−0.5, −1.6 to 0.5) | 0.951 | (0.0, −1.0 to 1.0) |
| p-value (Dif 95% CI) [2] | | | | | | |
| Irritability/Lability | | | | | | |
| (Domain Score) | | | | | | |
| Baseline: N, Mean (SD) | 62, 4.8 | (3.66) | 94, 5.1 | (3.62) | 97, 5.2 | (3.39) |
| Week 12 Change from Baseline: | −1.4 | (0.45) | −2.1 | (0.40) | −1.8 | (0.39) |
| LS Mean (SE) [2] | | | | | | |
| Treat Diff vs. Placebo: | | | 0.151 | (−0.7, −1.8 to 0.3) | 0.405 | (−0.4, −1.4 to 0.6) |
| p-value (Dif, 95% CI) [2] | | | | | | |
| NPI Total Score | | | | | | |
| Baseline: N, Meats (SD) | 62, 38.7 | (17.74) | 94, 39.4 | (18.93) | 97, 40.1 | (19.98) |
| Week 12 Change from Baseline: | −8.7 | (2.29) | −15.2 | (2.03) | −13.9 | (1.99) |
| LS Mean (SE) [2] | | | | | | |
| Treat Diff vs. Placebo: | | | 0.013 | (−6.6, −11.7 to −1.4) | 0.046 | (−5.2, −10.3 to −0.1) |
| p-value (Dif 95% CI) [2] | | | | | | |

Note:
Agitation/Aggression Domain-Caregiver Distress score ranges fro tn 0 to 5.
Aberrant Motor Behavior Domain score ranges from 0 to 12.
Irritability/Lability Domain score ranges from 0 to 12.
NPI Total score ranges from 0 to 144.
For all scores, higher scores indicate greater worsening condition.
[1] Patients who are randomized to the same treatment group in both stages.
[2] MMRM with fixed effects of treatment, visit, treatment-by-visit. Baseline, Baseline-by-visit, Baseline NPI A A (≤6 vs. >6), risk assessment for falls (normal/mild vs. moderate/severe). Baseline concomitant use of antipsychotic medications (yes vs. no). Unstructured variance-covariance was used.

### NPI—Agitation/Aggression Domain Score and Caregiver Distress Score

The overall SPCD p-value for the NP1-Agitation/Aggression Domain score for AVP-786-18 versus placebo was 0.695, and for AVP-786-28 versus placebo it was 0.904 (see Table 29a below).

The overall SPCD p-value for NPI—Agitation/Aggression Caregiver Distress score for AVP-786-18 versus placebo was 0.386, and for AVP-786-28 versus placebo it was 0.250 (Table 28).

For the 12-week Parallel Group, the treatment differences (CI) for the NPI-Agitation/Aggression Domain score for patients treated with AVP-786-18 and AVP-786-28 versus placebo were −0.5 (−1.5 to 0.5) and −0.6 (−1.6 to 0.4), respectively, with p=0.298 and p=0.214 (see Table 29b below). For the NPI—Agitation/Aggression Caregiver Distress score, there were no significant between-group differences at Week 12 (Table 29).

The rate of NPI—Agitation/Aggression responders was summarized with responders defined as patients with a 30% improvement from Baseline and separately with responders defined as patients with a 50% improvement from Baseline. There were no significant between-group differences in response rate using either response criteria in the overall SPCD analysis, in either stage individually, or in the 12-week Parallel Group analysis.

The changes from Baseline in the NPI Agitation/Aggression Domain scores at various time points are shown in the Tables 29a and 29b below:

TABLE 29a
NPI: Change from Baseline SPCD MMRM-Observed Data (mITT Population)
NPI: Agitation/Aggression Domain Score

| Stage | Parameter/Results | Placebo | | AVP-786 18 mg | | AVP-786 28 mg | |
|---|---|---|---|---|---|---|---|
| Stage 1 | Baseline: N, Mean (SD) | 191, 7.1 | (2.39) | 94, 6.5 | (1.94) | 97, 7.2 | (2.42) |
| | Week 1 Change from Baseline: | 185, −0.9 | (2.56) | 90, −1.1 | (2.27) | 94, −1.0 | (2.48) |
| | N, Mean (SD) | | | | | | |
| | Change from Baseline: | −0.7 | (0.23) | −1.1 | (0.29) | −0.8 | (0.29) |
| | LS Mean (SE) [1] | | | | | | |
| | Treat Diff vs. Placebo: | | | 0.182 | (−0.4, −1.0 to 0.2) | 0.830 | (−0.1, −0.6 to 0.5) |
| | p-value (Dif, 95% CI) [1] | | | | | | |
| | Week 2 Change from Baseline: | 141, −1.0 | (2.85) | 70, −1.6 | (2.63) | 70, −1.5 | (2.43) |
| | N, Mean (SD) | | | | | | |
| | Change from Baseline: | −0.7 | (0.25) | −1.7 | (0.32) | −1.3 | (0.32) |
| | LS Mean (SE) [1] | | | | | | |
| | Treat Diff vs. Placebo: | | | 0.002 | (−1.0, −1.7 to −0.4) | 0.059 | (−0.6, −1.3 to 0.0) |
| | p-value (Dif, 95% CI) [1] | | | | | | |
| | Week 3 Change from Baseline: | 187, −1.3 | (2.97) | 89, −1.8 | (2.95) | 95, −1.7 | (2.88) |
| | N, Mean (SD) | | | | | | |
| | Change from Baseline: | −1.1 | (0.25) | −2.0 | (0.33) | −1.5 | (0.32) |
| | LS Mean (SE) [1] | | | | | | |
| | Treat Diff vs. Placebo: | | | 0.017 | (−0.8, −1.5 to −0.2) | 0.299 | (−0.4, −1.0 to 0.3) |
| | p-value (Dif, 95% CI) [1] | | | | | | |
| | Week 6 Change from Baseline: | 183, −1.8 | (3.58) | 87, −2.0 | (3.03) | 94, −2.1 | (2.94) |
| | N, Mean (SD) | | | | | | |
| | Change from Baseline: | −1.6 | (0.27) | −2.1 | (0.36) | −1.9 | (0.35) |
| | LS Mean (SE) [1] | | | | | | |
| | Treat Diff vs. Placebo: | | | 0.182 | (−0.5, −1.3 to 0.2) | 0.390 | (−0.3, −1.1 to 0.4) |
| | p-value (Dif, 95% CI) [1] | | | | | | |
| Stage 2 | Stage 2 Baseline: | 40, 6.9 | (2.84) | 41, 6.3 | (2.71) | 44, 6.3 | (2.81) |
| (Stage 1 | N, Mean (SD) [2] | | | | | | |
| Placebo | Week 9 Change from Baseline: | 40, −1.5 | (2.91) | 41, −1.6 | (3.08) | 44, −0.6 | (2.90) |
| Non- | N, Mean (SD) | | | | | | |
| responders | Change from Baseline: | −1.3 | (0.42) | −1.6 | (0.41) | −0.7 | (0.40) |
| only) | LS Mean (SE) [1] | | | | | | |
| | Treat Diff vs. Placebo: | | | 0.570 | (−0.3, −1.5 to 0.8) | 0.275 | (0.6, −0.5 to 1.8) |
| | p-value (Dif, 95% CI) [1] | | | | | | |
| | Week 12 Change from Baseline: | 39, −1.7 | (3.00) | 41, −0.8 | (3.15) | 41, −0.9 | (3.35) |
| | N, Mean (SD) | | | | | | |
| | Change from Baseline: | −1.4 | (0.45) | −0.9 | (0.43) | −1.0 | (0.43) |
| | LS Mean (SE) [1] | | | | | | |
| | Treat Diff vs. Placebo: | | | 0.462 | (0.5, −0.8 to 1.7) | 0.528 | (0.4, −0.8 to 1.6) |
| | p-value (Dif, 95% CI) [1] | | | | | | |
| | SPCD Week 6 & 12 MMRM | | | −0.39, | p = 0.695 | −0.12, | p = 0.904 |
| | weighted z-statistic, p-value [3] | | | | | | |

Note:
Agitation/Aggression Domain Score ranges from 0 to 12 with higher scores indicating worsening condition.
[1] MMRMs include fixed effects of treatment, visit, treatment-by-visit, baseline, baseline-by-visit, and in the Stage 1 model, baseline NPI AA (≤6 vs. >6), risk assessment for falls (normal/mild vs. moderate/severe), baseline concomitant use of antipsychotic medications (yes vs. no). Unstructured variance-covariance was used. Stage 1 and 2 baseline values were used in their respective models.
[2] Stage 2 Baseline is the last non-missing assessment prior to Stage 2 re-randomization (re-randomization visit).
[3] SPCD Week 6 & 12 MMRM weighted z-statistic was used with weight = 0.6 for Stage 1 and 0.4 for Stage 2.

TABLE 29b
NPI: Change from Baseline Parallel Group MMRM Analysis-Observed Data
(mITT 12-Week Parallel Group Population)
NPI: Agitation/Aggression Domain Score

| Parameter/Results | Placebo [1] | | AVP-786 18 mg [1] | | AVP-786 28 mg [1] | |
|---|---|---|---|---|---|---|
| Baseline: N, Mean (SD) | 62, 7.2 | (2.52) | 94, 6.5 | (1.94) | 97, 7.2 | (2.42) |
| Week 1 Change from Baseline: | 60, −1.0 | (2.24) | 90, −1.1 | (2.27) | 94, −1.0 | (2.48) |
| N, Mean (SD) | | | | | | |
| Change from Baseline: | −0.7 | (0.34) | −1.0 | (0.30) | −0.8 | (0.30) |
| LS Mean (SE) [1] | | | | | | |
| Treat Diff vs. Placebo: | | | 0.381 | (−0.3, −1.1 to 0.4) | 0.788 | (−0.1, −0.8 to 0.6) |
| p-value (Dif, 95% CI) [2] | | | | | | |
| Week 2 Change from Baseline: | 48, −1.0 | (2.87) | 70, −1.6 | (2.63) | 70, −1.5 | (2.43) |
| N, Mean (SD) | | | | | | |
| Change from Baseline: | −0.8 | (0.38) | −1.6 | (0.33) | −1.3 | (0.33) |
| LS Mean (SE) [1] | | | | | | |
| Treat Diff vs. Placebo: | | | 0.070 | (−0.8, −1.7 to 0.1) | 0.314 | (−0.4, −1.3 to 0.4) |
| p-value (Dif, 95% CI) [2] | | | | | | |
| Week 3 Change from Baseline: | 61, −1.4 | (3.01) | 89, −1.8 | (2.95) | 95, −1.7 | (2.88) |
| N, Mean (SD) | | | | | | |
| Change from Baseline: | −1.1 | (0.39) | −1.9 | (0.34) | −1.5 | (0.34) |
| LS Mean (SE) [1] | | | | | | |
| Treat Diff vs. Placebo: | | | 0.086 | (−0.8, −1.7 to 0.1) | 0.447 | (−0.3, −1.2 to 0.5) |
| p-value (Dif, 95% CI) [2] | | | | | | |
| Week 6 Change from Baseline: | 60, −1.8 | (3.30) | 87, −2.0 | (3.03) | 94, −2.1 | (2.94) |
| N, Mean (SD) | | | | | | |
| Change from Baseline: | −1.5 | (0.42) | −2.0 | (0.37) | −2.0 | (0.36) |
| LS Mean (SE) [1] | | | | | | |
| Treat Diff vs. Placebo: | | | 0.301 | (−0.5, −1.5 to 0.5) | 0.330 | (−0.5, −1.4 to 0.5) |
| p-value (Dif, 95% CI) [2] | | | | | | |
| Week 9 Change from Baseline: | 57, −2.6 | (2.71) | 86, −2.6 | (3.07) | 92, −2.6 | (3.05) |
| N, Mean (SD) | | | | | | |
| Change from Baseline: | −2.3 | (0.40) | −2.7 | (0.35) | −2.4 | (0.34) |
| LS Mean (SE) [1] | | | | | | |
| Treat Diff vs. Placebo: | | | 0.459 | (−0.3, −1.3 to 0.6) | 0.833 | (−0.1, −1.0 to 0.8) |
| p-value (Dif, 95% CI) [2] | | | | | | |
| Week 12 Change from Baseline: | 57, −2.5 | (3.01) | 87, −2.7 | (3.16) | 91, −3.0 | (3.19) |
| N, Mean (SD) | | | | | | |
| Change from Baseline: | −2.2 | (0.42) | −2.7 | (0.36) | −2.8 | (0.36) |
| LS Mean (SE) [1] | | | | | | |
| Treat Diff vs. Placebo: | | | 0.298 | (−0.5. −1.5 to 0.5) | 0.214 | (−0.6, −1.6 to 0.4) |
| p-value (Dif, 95% CI) [2] | | | | | | |

Note:
Agitation/Aggression Domain Score ranges from 0 to 12 with higher scores indicating worsening condition.
[1] Patients who are randomized to the same treatment group in both stages.
[2] MMRM with fixed effects of treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no). Unstructured variance-covariance was used.

### NPI—Aberrant Motor Behavior Domain Score

The overall SPCD p-value for NPI—Aberrant Motor Behavior Domain score for AVP-786-18 versus placebo was 0.829, and for AVP-786-28 versus placebo it was 0.052 (Table 28).

For the 12-week Parallel Group, the treatment differences (CI) for patients treated with AVP-786-18 and AVP-786-28 versus placebo were −0.5 (−1.6 to 0.5) and 0.0 (−1.0 to 1.0), respectively, with p=0.307 and p=0.951 (Table 29).

### NPI—Irritability/Lability Domain Score

Patients treated with AVP-786-18 had a significantly (p=0.015) greater improvement in NPI—Irritability/Lability Domain score compared with the placebo group based on the overall SPCD analysis (Table 28). The overall SPCD p-value for AVP-786-28 versus placebo was not significant (p=0.163).

For the 12-week Parallel Group, the treatment differences (CI) for patients treated with AVP-786-18 and AVP-786-28 versus placebo were −0.7 (−1.8 to 0.3) and −0.4 (−1.4 to 0.6), respectively, with p=0.151 and p=0.405 (Table 29).

### NPI Total Score

The overall SPCD p-value for NPI Total score for AVP-786-18 versus placebo was 0.133, and for AVP-786-28 versus placebo it was 0.829 (Table 28).

For the 12-week Parallel Group, patients treated with AVP-786-18 and AVP-786-28 had significantly (p=0.013 and p=0.046, respectively) greater improvement in the NPI Total score compared with the placebo group (treatment differences [Cl] were −6.6 [−11.7 to −1.4] and −5.2 [−10.3 to −0.1] for AVP-786-18 versus placebo and AVP-786-28 versus placebo, respectively; Table 29).

### 5.3.1.4.3. NPI: Other Domains

All NPI domain scores are summarized in the tables elsewhere herein. Significant treatment differences in favor of AVP-786 versus placebo include:
1. NPI-Delusions Domain score, AVP-786-18, 12-week Parallel Group: p=0.038
2. NPI-Anxiety Domain score, AVP-786-18, 12-week Parallel Group: p=0.019
3. NPI-Anxiety Caregiver Distress score, AVP-786-18, SPCD and 12-week Parallel Group: p=0.006 and p=0.042, respectively
4. NPI—Apathy/Indifference Domain score, AVP-786-28, 12-week Parallel Group: p=0.031
5. NPI—Irritability/Lability Caregiver Distress score, AVP-786-18, SPCD: p=0.018
6. NPI—Aberrant Motor Behavior Caregiver Distress score, AVP-786-28, SPCD: p=0.015
7. NPI 4A Domain score, AVP-786-18, 12-week Parallel Group: p=0.042

### 5.3.1.4.4. CGIS—Agitation Score

The mean changes from Baseline in CGIS-Agitation score (Table 30) was significant in the overall SPCD analysis for AVP-786-18 versus placebo (p=0.049), but not for AVP-786-28 versus placebo (p=0.075).

TABLE 30
CGIS-Agitation Score: Change from Baseline SPCD ANCOVA-LOCF Data
(mITT Population)

| Stage | | | | | | |
|---|---|---|---|---|---|---|
| Parameter/Results | Placebo | | AVP-786-18 | | AVP-786-28 | |
| Stage 1 | | | | | | |
| Baseline: N, Mean (SD) | 191, 4.5 | (0.64) | 94, 4.3 | (0.49) | 97, 4.4 | (0.70) |
| Week 6: N, Mean (SD) | 191, 3.9 | (0.94) | 94, 3.6 | (0.90) | 97, 3.7 | (1.04) |
| Week 6 Change from Baseline: SES | | | −0.143 | | −0.150 | |
| % Change from Baseline: Mean | −12.2 | | −14.8 | | −14.7 | |
| Week 6 Change from Baseline: | −0.6 | (0.09) | −0.7 | (0.11) | −0.7 | (0.11) |
| LS Mean (SE) [1] | | | | | | |
| ANCOVA LS Mean Diff vs. Placebo: | | | 0.118 | (−0.2, −0.4 to 0.0) | 0.191 | (−0.1, −0.4 to 0.1) |
| p-value (Dif, 95% CI) [1] | | | | | | |
| Stage 2 | | | | | | |
| (Placebo Nonresponders only) | | | | | | |
| Baseline: N, Mean (SD) [2] | 40, 4.4 | (0.87) | 41, 4.2 | (0.77) | 44, 4.1 | (0.77) |
| Week 12: N, Mean (SD) | 40, 4.2 | (1.02) | 41, 3.8 | (0.78) | 44, 3.8 | (1.06) |
| Week 12 Grange from Baseline: SES | | | −0.220 | | −0.139 | |
| % Change from Baseline: Mean | −5.1 | | −7.5 | | −6.9 | |
| Week 12 Change from Baseline: | −0.2 | (0.12) | −0.4 | (0.12) | −0.4 | (0.12) |
| LS Mean (SE) [1] | | | | | | |
| ANCOVA LS Mean Diff vs. Placebo: | | | 0.225 | (−0.2, −0.5 to 0.1) | 0.227 | (−0.2, −0.5 to 0.1) |
| p-value (Dif. 95% CI) [1] | | | | | | |
| SPCD OLS weighted z-statistic, | | | −1.97, | p = 0.049 | −1.78, | p = 0.075 |
| p-value [3] | | | | | | |

Note:
The CGIS-Agitation scale ranges from 1 to 7 with higher scores indicating worsening condition.
[1] Treatment effects were estimated at each stage by ANCOVA with fixed effects for treatment. Baseline, and in the Stage 1 model, Baseline NPI AA (≤ 6 vs >6), risk assessments for falls (normal/mild vs moderate/severe). Baseline concomitant use of antipsychotic medications (yes vs no). Stage 1 and 2 models use their respective Baseline values. Missing values were imputed by LOCF within each stage.
[2] Stage 2 Baseline is the last nonmissing assessment prior to Stage 2 rerandomization (rerandomization visit).
[3] OLS weighted z-statistic was used with weight = 0.6 for Stage 1 and 0.4 for Stage 2.

For the 12-week Parallel Group, the mean differences at Week 12 were not significantly greater than placebo for either AVP-786 treatment group.

### 5.3.1.4.5. ADCS-CGIC-Overall Score

The overall SPCD p-values for the ADCS-CGIC-Overall score were 0.063 for AVP-786-18 versus placebo and 0.115 for AVP-786-28 versus placebo (Table 31).

TABLE 31
ADCS-CGIC-Overall Score: SPCD ANCOVA-LOCF Data
(mITT Population)

| Stage | | | | | | |
|---|---|---|---|---|---|---|
| Parameter/Results | Placebo | | AVP-786-18 | | AVP-786-28 | |
| Stage 1 | | | | | | |
| N | 183 | | 90 | | 96 | |
| 1 = Marked Improvement, n (%) | 1 | (0.5) | 1 | (1.1) | 4 | (4.2) |
| 2 = Moderate improvement, n (%) | 20 | (10.9) | 16 | (17.8) | 11 | (11.5) |
| 3 = Minimal Improvement, n (%) | 64 | (35.0) | 31 | (34.4) | 30 | (31.3) |
| 4 = No change, it (%) | 69 | (37.7) | 29 | (32.2) | 34 | (35.4) |
| 5 = Minimal Worsening, n (%) | 21 | (11.5) | 8 | (8.9) | 14 | (14.6) |
| 6 = Moderate Worsening, n (%) | 7 | (3.8) | 4 | (4.4) | 3 | (3.1) |
| 7 = Marked Worsening, n (%) | 1 | (0.5) | 1 | (1.1) | 0 | |
| Week 6 Score: Mean (SD) | 3.6 | (1.01) | 3.5 | (1.12) | 3.5 | (1.10) |
| Week 6 Score: LS Mean (SE) [1] | 3.7 | (0.11) | 3.5 | (0.14) | 3.6 | (0.13) |
| ANCOVA LS Mean Diff vs. Placebo: | | | 0.364 | (−0.1, −0.4 to 0.1) | 0.427 | (−0.1, −0.4 to 0.2) |
| p-value (Dif, 95% CI) [1] | | | | | | |
| Stage 2 | | | | | | |
| (Placebo Nonresponders only) | | | | | | |
| N | 40 | | 40 | | 42 | |
| 1 = Marked Improvement, n (%) | 0 | | 0 | | 1 | (2.4) |
| 2 = Moderate Improvement, n (%) | 1 | (2.5) | 3 | (7.5) | 6 | (14.3) |
| 3 = Minimal Improvement, n (%) | 9 | (22.5) | 18 | (45.0) | 12 | (28.6) |
| 4 = No change, n (%) | 18 | (45.0) | 13 | (32.5) | 14 | (33.3) |
| 5 = Minimal Worsening, n (%) | 11 | (27.5) | 3 | (7.5) | 5 | (11.9) |
| 6 = Moderate Worsening, n (%) | 1 | (2.5) | 3 | (7.5) | 3 | (7.1) |
| 7 = Marked Worsening, n (%) | 0 | | 0 | | 1 | (2.4) |
| Week 12 Score Relative to Week 6: | 4.1 | (0.85) | 3.6 | (1.00) | 3.7 | (1.28) |
| Mean (SD) | | | | | | |
| Week 12 Score Relative to Week 6: | 4.0 | (0.17) | 3.6 | (0.17) | 3.7 | (0.16) |
| LS Mean (SE) [1] | | | | | | |
| ANCOVA LS Mean Diff vs. Placebo: | | | 0.098 | (−0.4, −0.9 to 0.1) | 0.168 | (−0.3, −0.8 to 0.1) |
| p-value (Dif, 95% CI) [1] | | | | | | |
| SPCD OLS weighted z-statistic, | | | −1.86, | p = 0.063 | −1.57, | p = 0.115 |
| p-value [2] | | | | | | |

Note:
ADCS-CGIC-Overall scores range from 1 to 7 with lower scores indicating improvement.
[1] Treatment effects were estimated at each stage by ANCOVA with fixed effects for treatment, Baseline CMAI Total score, and in the Stage 1 model, Baseline NPIAA (≤6 vs >6), risk assessments for falls (normal/mild vs moderate/severe), Baseline concomitant use of antipsychotic medications (yes vs no). Stage 1 and 2 Baseline values were used in their respective models. Missing values were imputed by LOCF within each stage.
[2] OLS weighted z-statistic was used with weight = 0.6 for Stage 1 and 0.4 for Stage 2.

For the 12-week Parallel Group, the between-group differences at Week 12 were not significant for either AVP-786 treatment group compared to placebo.

### 5.3.1.4.6. PGIC Score

The overall SPCD p-value for the PGIC score was significant for AVP-786-18 versus placebo (p=0.003), but not for AVP-786-28 vs placebo (p=0.073), as shown in Table 32.

TABLE 32
PGIC Score: SPCD ANCOVA-LOCF Data (mITT Population)

| Stage | | | | | | |
|---|---|---|---|---|---|---|
| Parameter/Resalts | Placebo | | AVP-786-18 | | AVP-786-28 | |
| Stage 1 | | | | | | |
| N | 185 | | 90 | | 96 | |
| 1 = Very much improved, n (%) | 4 | (2.2) | 8 | (8.9) | 6 | (6.3) |
| 2 = Much improved, n (%) | 34 | (18.4) | 18 | (20.0) | 18 | (18.8) |
| 3 = Minimally improved, n (%) | 67 | (36.2) | 37 | (41.1) | 35 | (36.5) |
| 4 = No change, n (%) | 54 | (29.2) | 20 | (22.2) | 26 | (27.1) |
| 5 = Minimally worse, a (%) | 17 | (9.2) | 4 | (4.4) | 10 | (10.4) |
| 6 = Much worse, n (%) | 8 | (4.3) | 2 | (2.2) | 1 | (1.0) |
| 7 = Very much worse, rt (%) | 1 | (0.5) | 1 | (1.1) | 0 | |
| Week 6 Score: LS Mean (SE) [1] | 3.4 | (0.12) | 3.0 | (0.15) | 3.1 | (0.14) |
| ANCOVA LS Mean Diff vs. Placebo: | | | 0.014 | (−0.4, −0.6 to −0.1) | 0.111 | (−0.2, −0.5 to 0.1) |
| p-value (Dif, 95% CI) [1] | | | | | | |
| Stage 2 | | | | | | |
| (Placebo Nonresponders only) | | | | | | |
| N | 40 | | 41 | | 44 | |
| 1 = Very much improved, n (%) | 1 | (2.5) | 3 | (7.3) | 2 | (4.5) |
| 2 = Much improved, n (%) | 6 | (15.0) | 8 | (19.5) | 12 | (27.3) |
| 3 = Minimally improved, n (%) | 14 | (35.0) | 17 | (41.5) | 14 | (31.8) |
| 4 = No change, n (%) | 9 | (22.5) | 9 | (22.0) | 8 | (18.2) |
| 5 = Minimally worse, n (%) | 6 | (15.0) | 4 | (9.8) | 4 | (9.1) |
| 6 = Much worse, n (%) | 4 | (10.0) | 0 | | 3 | (6.8) |
| 7 = Very much worse, n (%) | 0 | | 0 | | 1 | (2.3) |
| Week 12 Score Relative to Baseline: | 3.6 | (0.20) | 3.1 | (0.20) | 3.3 | (0.19) |
| LS Mean (SE) [1] | | | | | | |
| ANCOVA LS Mean Diff vs. Placebo: | | | 0.062 | (−0.5, −1.1 to 0.0) | 0.305 | (−0.3, −0.8 to 0.3) |
| p-value (Dif, 95% CI) [1] | | | | | | |
| SPCD OLS weighted z-statistic, | | | −3.01, | p = 0.003 | −1.79, | p = 0.073 |
| p-value [2] | | | | | | |

Note:
PGIC scores range from 1 to 7 with lower scores indicating improvement.
[1] Treatment effects were estimated at each stage by ANCOVA with fixed effects for treatment. Baseline CMAI Total score, and in the Stage 1 model, Baseline NPI AA (≤6 vs >6), risk assessments for falls (normal/mild vs moderate/severe). Baseline concomitant use of antipsychotic medications (yes vs no). Stage 1 and 2 Baseline values were used in their respective models. Missing values were imputed by LOCF within each stage.
[2] OLS weighted z-statistic was used with weight = 0.6 for Stage 1 and 0.4 for Stage 2.

For the 12-week Parallel Group, the between-group differences at Week 12 were not significant for either AVP-786 treatment group compared to placebo. A PGIC response summary (with patients reporting much improved or very much improved counted as responders) is presented for the 12 week Parallel Group.

### 5.3.1.4.7. Zarit Burden Interview

The overall SPCD p-values for the ZBI were 0.502 for AVP-786-18 versus placebo and 0.564 for AVP-786-28 versus placebo (Table 33).

TABLE 33
ZBI Total Score: Change from Baseline SPCD ANCOVA-LOCF Data
(mITT Population)

| Stage | | | | | | |
|---|---|---|---|---|---|---|
| Parameter/Results | Placebo | | AVP-786-18 | | AVP-786-28 | |
| Stage 1 | | | | | | |
| Baseline: N, Mean (SD) | 190, 30.9 | (16.19) | 94, 30.0 | (16.96) | 97, 33.0 | (16.68) |
| Week 6: N, Mean (SD) | 191, 29.5 | (17.02) | 94, 28.0 | (16.74) | 97, 30.2 | (15.42) |
| Week 6 Change from Baseline: SES | | | −0.070 | | −0.149 | |
| % Change from Baseline: Mean | −3.2 | | −3.9 | | −2.4 | |
| Week 6 Change from Baseline: | −1.7 | (0.86) | −2.3 | (1.08) | −2.7 | (1.07) |
| LS Mean (SE) [1] | | | | | | |
| ANCOVA LS Mean Diff vs. Placebo: | | | 0.530 | (−0.7, −2.7 to 1.4) | 0.326 | (−1.0, −3.1 to 1.0) |
| p-value (Dif, 95% CI) [1] | | | | | | |
| Stage 2 | | | | | | |
| (Placebo Nonresponders only) | | | | | | |
| Baseline: N, Mean (SD) [2] | 40, 31.5 | (15.24) | 41, 32.0 | (16.57) | 44, 28.6 | (16.76) |
| Week 12: N, Mean (SD) | 40, 30.4 | (14.17) | 41, 33.3 | (16.97) | 44, 28.1 | (16.73) |
| Week 12 Grange from Baseline: SES | | | 0.301 | | 0.079 | |
| % Change from Baseline: Mean | −0.4 | | 16.6 | | 2.2 | |
| Week 12. Change from Baseline: | −1.0 | (1.20) | 1.5 | (1.18) | −0.8 | (1.14) |
| LS Mean (SE) [1] | | | | | | |
| ANCOVA LS Mean Diff vs. Placebo: | | | 0.135 | (2.5, −0.8 to 5.9) | 0.895 | (0.2, −3.1 to 3.5) |
| p-value (Dif, 95% CI) [1] | | | | | | |
| SPCD OLS weighted z-statistic, | | | 0.67, | p = 0.502 | −0.58, | p = 0.564 |
| p-value [3] | | | | | | |

Note:
ZBI Total score ranges from 0 to 88 with higher scores indicating greater caregiver burden.
[1] Treatment effects were estimated at each stage by ANCOVA with fixed effects for treatment, Baseline, and in the Stage 1 model, Baseline NPI AA (≤ 6 vs >6), risk assessments for falls (normal/mild vs moderate/severe). Baseline concomitant use of antipsychotic medications (yes vs no). Stage 1 and 2 models use their respective Baseline values. Missing values were imputed by LOCF within each stage.
[2] Stage 2 Baseline is the last nonmissing assessment prior to Stage 2 rerandomization (rerandomization visit).
[3] OLS weighted z-statistic was used with weight = 0.6 for Stage 1 and 0.4 for Stage 2.

For the 12-week Parallel Group, the mean differences from placebo at Week 12 were not significant for either AVP-786 treatment group

### 5.3.1.4.8. DEMQOL Total Score

The overall SPCD p-values for the DEMQOL Total score were 0.456 for AVP-786-18 vs placebo and 0.678 for AVP-786-28 versus placebo (Table 34).

TABLE 34
DEMQOL Total Score: Change from Baseline SPCD ANCOVA-LOCF
Data (mITT Population)

| Stage | | | | | | |
|---|---|---|---|---|---|---|
| Parameter/Results | Placebo | | AVP-786-18 | | AVP-786-28 | |
| Stage 1 | | | | | | |
| Baseline: N, Meats (SD) | 133, 85.7 | (10.63) | 69, 82.9 | (10.22) | 72, 84.0 | (11.98) |
| Week 6: N,Mean(SD) | 133, 88.0 | (10.46) | 69, 86.4 | (10.54) | 72, 88.4 | (10.22) |
| Week 6 Change from Baseline: SES | | | 0.150 | | 0.235 | |
| % Change from Baseline: Mean | 3.2 | | 4.9 | | 6.6 | |
| Week 6 Change from Baseline: | 2.3 | (0.98) | 2.5 | (1.22) | 3.8 | (1.21) |
| LS Mean (SE) [1] | | | | | | |
| ANCOVA LS Mean Diff vs. Placebo: | | | 0.909 | (0.1, −2.2 to 2.5) | 0.226 | (1.4, −0.9 to 3.8) |
| p-value (Dif, 95% CI) [1] | | | | | | |
| Stage 2 | | | | | | |
| (Stage 1 Placebo Nonresponders only) | | | | | | |
| Baseline: N, Mean (SD) [2] | 24, 86.8 | (9.96) | 30, 90.0 | (9.51) | 32, 87.8 | (10.72) |
| Week 12: N, Mean (SD) | 25, 89.5 | (9.72) | 30, 93.1 | (9.40) | 32, 88.9 | (11.96) |
| Week 12 Change from Baseline: SES | | | 0.080 | | −0.131 | |
| % Change from Baseline: Mean | 3.1 | | 4.2 | | 2.1 | |
| Week 12 Change from Baseline: | 1.8 | (1.86) | 3.9 | (1.67) | 0.9 | (1.61) |
| LS Mean (SE) [1] | | | | | | |
| ANCOVA LS Mean Diff vs. Placebo: | | | 0.405 | (2.1, −2.9 to 7.1) | 0.714 | (−0.9, −5.8 to 4.0) |
| p-value (Dif, 95% CI) [1] | | | | | | |
| SPCD OLS weighted z-statistic, | | | 0.75, | p = 0.456 | 0.42, | p = 0.678 |
| p-value [3] | | | | | | |

Note:
The DEMQOL Total score ranges from 28 to 112 with higher scores indicating greater QOL.
[1] Treatment effects were estimated at each stage by ANCOVA with fixed effects for treatment, Baseline, and in the Stage 1 model, Baseline NPI AA (≤6 vs >6), risk assessments for fails (normal/mild vs moderate/severe). Baseline concomitant use of antipsychotic medications (yes vs no). Stage 1 and 2 models use their respective Baseline values. Missing values were imputed by LOCF within each stage.
[2] Stage 2 Baseline is the last nonmissing assessment prior to Stage 2 rerandomization (rerandomization visit).
[3] OLS weighted z-statistic was used with weight = 0.6 for Stage 1 and 0.4 for Stage 2.

For the DEMQOL-Proxy Total score (completed by caregivers), the overall SPCD p-value was significant for both AVP-786-18 versus placebo (p=0.002) and AVP-786-28 versus placebo (p=0.019) (see Table 35).

TABLE 35
DEMQOL-proxy: Change from Baseline SPCD ANCOVA-LOCF Data
(mITT Population)

| Stage Parameter/Results | Placebo | | AVP-786-18 | | AVP-786-28 | |
|---|---|---|---|---|---|---|
| Stage 1 | | | | | | |
| Baseline: N, Meats (SD) | 191, 95.4 | (12.93) | 94, 93.8 | (13.99) | 97, 93.9 | (13.07) |
| Week 6: N,Mean(SD) | 191, 98.3 | (13.17) | 94, 98.4 | (11.21) | 97, 98.4 | (13.40) |
| 'Week 6 Change from Baseline: SES | | | 0.167 | | 0.163 | |
| % Change from Baseline: Mean | 3.6 | | 6.2 | | 5.5 | |
| Week 6 Change from Baseline: | 2.7 | (0.98) | 3.9 | (1.22) | 3.9 | (1.20) |
| LS Mean (SE) [1] | | | | | | |
| ANCOVA LS Mean Diff vs. Placebo: | | | 0.314 | (1.2, −1.1 to 3.5) | 0.320 | (1.2, −1.1 to 3.5) |
| p-value (Dif, 95% CI) [1] | | | | | | |
| Stage 2 | 40, 97.2 | (13.58) | 41, 98.5 | (13.60) | 44, 99.0 | (12.46) |
| (Placebo Nonresponders only) | | | | | | |
| Week 12: N, Mean (SD) | 40, 94.2 | (17.33) | 41, 102.4 | (14.38) | 44, 100.6 | (13.35) |
| Week 12 Change from Baseline: SES | | | 0.635 | | 0.470 | |
| % Change from Baseline: Mean | −3.2 | | 4.6 | | 2.0 | |
| Week 12 Change from Baseline: | −3.1 | (1.60) | 4.0 | (1.58) | 1.7 | (1.53) |
| LS Mean (SE) [1] | | | | | | |
| ANCOVA LS Mean Diff vs. Placebo: | | | 0.002 | (7.1, 2.6 to 11.5) | 0.030 | (4.9, 0.5 to 9.2) |
| p-value (Dif, 95% CI) [1] | | | | | | |
| SPCD OLS weighted z-statistic, | | | 3.10, | p = 0.002 | 2.34, | p = 0.019 |
| p-value [3] | | | | | | |

Note:
The DEMQOL-proxy scale ranges from 31 to 124 with higher scores indicating greater QOL.
[1] Treatment effects were estimated at each stage by ANCOVA with fixed effects for treatment, Baseline, and in the Stage 1 model. Baseline NPI AA (≤6 vs >6), risk assessments for falls (normal/mild vs moderate/severe). Baseline concomitant use of antipsychotic medications (yes vs no). Stage 1 and 2 models use their respective Baseline values. Missing values were imputed by LOCF within each stage.
[2] Stage 2 Baseline is the last nonmissing assessment prior to Stage 2 rerandomization (rerandomization visit).
[3] OLS weighted z-statistic was used with weight = 0.6 for Stage 1 and 0.4 for Stage 2.

For the 12-week Parallel Group, for both the DEMQOL Total score and the DEMQOL-Proxy Total score, the mean differences from placebo at Week 12 were not significant for either AVP 786 treatment group.

### 5.3.1.4.9. CSDD Score

The overall SPCD p-values for the CSDD score were 0.213 for AVP-786-18 versus placebo and 0.985 for AVP-786-28 versus placebo.

TABLE 36
CSDD Score: Change from Baseline SPCD ANCOVA-LOCF Data
(mITT Population)

| Stage | | | | | | |
|---|---|---|---|---|---|---|
| Parameter/Results | Placebo | | AVP-786-18 | | AVP-786-28 | |
| Stage 1 | | | | | | |
| Baseline: N, Mean (SD) | 191, 6.2 | (2.24) | 94, 6.3 | (2.11) | 97, 6.4 | (2.19) |
| Week 6: N, Mean (SD) | 191, 5.9 | (3.07) | 94, 5.5 | (3.16) | 97, 5.9 | (3.16) |
| Week 6 Change from Baseline: SES | | | −0.142 | | −0.057 | |
| % Change from Baseline: Mean | 4.9 | | −5.0 | | −0.0 | |
| Week 6 Change from Baseline: | −0.4 | (0.30) | −0.8 | (0.37) | −0.5 | (0.37) |
| LS Mean (SE) [1] | | | | | | |
| ANCOVA LS Mean Diff vs. Placebo: | | | 0.278 | (−0.4, −1.1 to 0.3) | 0.817 | (−0.1, −0.8 to 0.6) |
| p-value (Dif, 95% CI) [1] | | | | | | |
| Stage 2 | | | | | | |
| (Stage 1 Placebo Nonresponders only) | | | | | | |
| Baseline: N Mean (SD) [2] | 40, 6.6 | (3.89) | 41, 5.9 | (2.78) | 44. 6.8 | (2.84) |
| Week 12: N, Mean (SD) | 40, 6.2 | (2.99) | 41, 5.4 | (2.82) | 44, 6.5 | (3.57) |
| Week 12 Change from Baseline: SES | | | −0.056 | | 0.011 | |
| % Change from Baseline: Mean | 7.2 | | −1.5 | | 0.4 | |
| Week 12 Change from Baseline: | −0.3 | (0.39) | −0.7 | (0.38) | −0.2 | (0.37) |
| LS Mean (SE) [1] | | | | | | |
| ANCOVA LS Mean Diff vs. Placebo: | | | 0.500 | (−0.4, −1.4 to 0.7) | 0.795 | (0.1, −0.9 to 1.2) |
| p-value (Dif, 95% CI) [1] | | | | | | |
| SPCD OLS weighted z-statistic, | | | −1.25, | p = 0.213 | 0.02, | p = 0.985 |
| p-value [3] | | | | | | |

Note:
The CSDD score ranges from 0 to 38 with higher scores indicating evidence of depression.
[1] Treatment effects were estimated at each stage by ANCOVA with fixed effects for treatment, Baseline, and in the Stage 1 model, Baseline NPI AA (≤6 vs >6), risk assessments for falls (normal/mild vs moderate/severe). Baseline concomitant use of antipsychotic medications (yes vs no). Stage 1 and 2 models use their respective Baseline values. Missing values were imputed by LOCF within each stage.
[2] Stage 2 Baseline is the last nonmissing assessment prior to Stage 2 rerandomization (rerandomization visit).
[3] OLS weighted z-statistic was used with weight = 0.6 for Stage 1 and 0.4 for Stage 2.

For the 12-week Parallel Group, the mean differences from placebo at Week 12 were not significant for either AVP-786 treatment group.

### 5.3.1.4.10. GMHR

The GMHR was only performed at Baseline and Week 12. For the 12-week Parallel Group, the proportions of patients with ratings of “excellent to very good” and “good” combined were 82.3%, 78.7%, and 83.5% for placebo, AVP-786-18 and AVP-786-28, respectively.

### 5.3.1.4.11. ADAS-Cog

The overall SPCD p-values for the ADAS-cog were 0.471 for AVP-786-18 versus placebo and 0.618 for AVP-786-28 versus placebo (Table 37).

TABLE 37
ADAS-cog: Change from Baseline SPCD ANCOVA - LOCF Data
(mITT Population)

| Stage | | | | | | |
|---|---|---|---|---|---|---|
| Parameter/Results | Placebo | | AVP-786-18 | | AVP-786-28 | |
| Stage 1 | | | | | | |
| Baseline: N, Meats (SD) | 131, 25.8 | (10.10) | 68, 26.2 | (8.99) | 71, 25.3 | (8.88) |
| Week 6: N, Mean (SD) | 132, 25.6 | (10.47) | 68, 24.1 | (8.91) | 71, 24.7 | (9.76) |
| Week 6 Change from Baseline: SES | | | −0.358 | | −0.014 | |
| % Change from Baseline: Mean | −0.6 | | −6.5 | | −1.2 | |
| Week 6 Change from Baseline: | −0.6 | (0.54) | −2.2 | (0.67) | −0.7 | (0.67) |
| LS Mean (SE) [1] | | | | | | |
| ANCOVA LS Mean Diff vs. Placebo: | | | 0.018 | (−1.6, −2.9 to −0.3) | 0.956 | (−0.0, −1.3 to 1.3) |
| p-value (Dif, 95% CI) [1] | | | | | | |
| Stage 2 | | | | | | |
| (Stage 1 Placebo Nonresponders only) | | | | | | |
| Baseline: N, Mean (SD) [2] | 25, 26.9 | (9.56) | 27, 27.0 | (9.27) | 32, 25.3 | (10.91) |
| Week 12: N. Mean (SD) | 25, 26.1 | (11.19) | 29, 27.5 | (10.18) | 32, 25.4 | (11.99) |
| Week 12 Change from Baseline: SES | | | 0.220 | | 0.170 | |
| % Change from Baseline: Mean | −2.2 | | 0.8 | | 2.2 | |
| Week 12 Change from Baseline: | −0.8 | (1.04) | 0.3 | (1.00) | 0.1 | (0.92) |
| LS Mean (SE) [1] | | | | | | |
| ANCOVA LS Mean Diff vs. Placebo: | | | 0.451 | (1.1, −1.8 to 4.0) | 0.519 | (0.9, −1.9 to 3.7) |
| p-value (Dif, 95% CI) [1] | | | | | | |
| SPCD OLS weighted z-statistic, | | | −0.72, | p = 0.471 | 0.50, | p = 0.618 |
| p-value [3] | | | | | | |

Note:
ADAS-cog scores range from 0 to 70 with higher scores indicating greater cognitive impairment.
[1] Treatment effects were estimated at each stage by ANCOVA with fixed effects for treatment, Baseline, and in the Stage 1 model, Baseline NPI AA (≤6 vs >6), risk assessments for fails (normal/mild vs moderate/severe). Baseline concomitant use of antipsychotic medications (yes vs no). Stage 1 and 2 models use their respective Baseline values. Missing values were imputed by LOCF within each stage.
[2] Stage 2 Baseline is the last nonmissing assessment prior to Stage 2 retandomization (rerandomization visit).
[3] OLS weighted z-statistic was used with weight = 0.6 for Stage 1 and 0.4 for Stage 2.

For the 12-week Parallel group, the mean differences from placebo at Week 12 were not significant for either AVP-786 treatment group. There did not appear to be any worsening of cognition for the AVP-786 treatment groups compared to placebo.

### 5.3.1.4.12. RUD

In general, there were few differences between treatment groups in RUD at Week 6 or Week 12. The amount of time per day and the number of days the caregiver spent assisting the patient were similar in all treatment groups. Among caregivers who worked for pay, the proportion who reported that their responsibilities as caregiver affected their work at Week 12 was lowest in the AVP-786-18 group (24.2%) and highest in the placebo group (44.0%), with the AVP-786-28 group being intermediate (35.3%), but the numbers of caregivers reporting in each group were low. The proportion of caregivers who visited health care professionals was also lowest in the AVP-786-18 group (26.7%) and highest in the placebo group (43.1%), with the AVP-786-28 group being intermediate (37.0%).

### 5.3.2. Statistical/Analytical Issues

Statistical and analytical issues are addressed briefly below.

### 5.3.2.1. Use of an “Efficacy Subset” of Patients

The primary analysis was performed on the mITT population, defined as all randomized patients with at least 1 postbaseline efficacy assessment.

Overall, few clinically meaningful differences were observed in CMAI Total score by subgroup. In the overall SPCD analysis, the patterns of results were similar to the primary results (i.e., notably greater improvement from Baseline to Week 12 for the AVP-786-18 group with the AVP-786-28 group being comparable to the placebo group) for patients with and without Baseline psychotropic concomitant medications that are major CYP2D6 substrates, but the group differences were smaller among patients without Baseline psychotropic concomitant medications that are major CYP2D6 substrates. The differences were not significant in either group. The same pattern was observed in the 12-week Parallel Group.

This same pattern was also apparent for patients with and without Baseline beta blocker concomitant medications for the overall SPCD analysis. In the 12-week Parallel Group, the same pattern emerged among patients with Baseline beta blocker concomitant medications, but there were too few patients without Baseline beta blocker concomitant medications to make a meaningful comparison (N=4, 9, and 6 for placebo, AVP-786-18, and AVP-786-28, respectively).

Among patients with CMAI factor 1 agitated status at Baseline (n=143, 64, and 66 in the placebo, AVP-786-18, and AVP-786-28 groups, respectively), the improvement from Baseline was significantly greater for AVP-786-18 compared with placebo (p=0.006 in the overall SPCD analysis) but not for AVP-786-28 (p=0.168). Similarly, in the 12-week Parallel Group (n=46, 64, and 66), at Week 12 the improvement was greater for AVP-786-18 compared with placebo (p=0.042) but not for not for AVP-786-28 (p=0.555).

It was also determined that AVP 786-18 provided a significant treatment difference, as determined by a reduction in the CMAI total score, in patients that had an CMAI aggressive behavior score of greater than 15 prior to administration of therapeutically effective amounts of d6-DM and quinidine sulfate.

Among patients not using antipsychotic medications at Baseline (n=122, 60, and 65 in the placebo, AVP-786-18, and AVP-786-28 groups, respectively), the improvements from Baseline in CMAI Total score were similar to those in the population as a whole. Baseline was not significantly different than placebo in either the AVP-786-18 (p=0.464 in the overall SPCD analysis) or AVP-786-28 (p=0.576) groups. Similarly, in the 12-week Parallel Group (n=37, 55, and 62), at Week 12 the improvement was not significantly different than placebo in either the AVP-786-18 (p=0.431 in the overall SPCD analysis) or AVP-786-28 (p=0.421) groups.

### 5.3.3. Efficacy Conclusions

Primary Efficacy Endpoint (CMAI Total Score):
SPCD Analysis: For the overall SPCD analysis, patients treated with AVP-786-18 showed greater improvement in the mean CMAI Total score compared with placebo (significant at the nominal level, p=0.008). No significant improvement in the mean CMAI Total score was observed for patients treated with AVP-786-28 compared with placebo (p=0.208).
   In Stage 1, patients treated with AVP-786-18 showed greater improvement in the mean CMAI Total score compared with placebo (significant at the nominal level, p=0.021). No significant improvement in the mean CMAI Total score was observed for patients treated with AVP-786-28 compared with placebo (p=0.731).
   In Stage 2, patients treated with AVP-786-18 and AVP-786-28 showed greater improvement in the mean CMAI Total score compared with placebo; however, the treatment difference did not reach significance at the nominal level (p=0.157 and p=0.150, respectively).
12-week Parallel Group: Patients treated with AVP-786-18 showed greater improvement in the mean CMAI Total score compared with placebo (significant at the nominal level, p=0.042). No significant improvement was observed in the mean CMAI Total score for patients treated with AVP-786-28 compared with placebo (p=0.555).
Sensitivity Analyses: Results from the sensitivity analyses supported the primary analysis, with AVP-786-18 being significantly better than placebo by the SUR method, SPCD ANCOVA−LOCF, SPCD ANCOVA−WOCF+LOCF, and MMRM SPCD using ITT population.

Key Secondary Efficacy Endpoint (mADCS-CGIC-Agitation Score):
For the SPCD analysis, patients treated with AVP-786-18 showed greater improvement in the mean mADCS-CGIC-Agitation score compared with placebo (significant at the nominal level, p=0.012). No significant improvement was observed in mean mADCS-CGIC-Agitation score for patients treated with AVP-786-28 compared with placebo (p=0.097).

All Secondary Efficacy Endpoints:

Results of the overall SPCD comparison of each AVP-786 treatment group versus placebo are summarized in Table 38 for all secondary efficacy endpoints. The primary efficacy endpoint is also included for completeness.
Among the secondary efficacy endpoints, patients treated with AVP-786-18 showed significant improvement compared with placebo (p<0.05) in all the subscales of the CMAI (F1—Aggressive Behavior, F2—Physically Nonaggressive Behavior, and F3—Verbally Agitated Behavior), NPI—Irritability/Lability Domain score, CGIS-Agitation score, PGIC score, and DEMQOL-proxy. Patients treated with AVP-786-28 showed significant improvement compared with placebo on the DEMQOL-proxy (p=0.019).
Similar findings were observed for the 12-week Parallel Group, but the comparisons for patients treated with AVP-786-18 and AVP-786-28 compared with placebo did not generally reach statistical significance. Patients treated with AVP-786-18 showed significant improvement compared with placebo on the CMAI subscale F2—Physically Nonaggressive Behavior (p=0.003) and the NPI Total (p=0.013). Patients treated with AVP-786-28 showed significant improvement compared with placebo on the NPI Total (p=0.046).

TABLE 38
Overall Summary of Results by Efficacy Endpoints (mITT Population)

| | SPCD Analysis | |
|---|---|---|
| Endpoint | AVP-786-18 | AVP-786-28 |
| Primary Efficacy | | |
| CMAI Total score | p = 0.008 | — |
| CMAI F1-Aggressive Behavior | p = 0.018 | — |
| CMAI F2-Physically Nonaggtessive | p = 0.017 | — |
| Behavior | | |
| CMAI F3-Verbally Agitated Behavior | p = 0.044 | — |
| Key Secondary Efficacy | | |
| mADCS-CGIC-Agitation Score | p = 0.012 | — |
| Secondary Efficacy | | |
| NPI-Agitation/Aggression Domain | — | — |
| score | | |
| NPI-Agitation/Aggression Caregiver | — | — |
| Distress | | |
| NPI-Abetrant Motor Behavior | — | — |
| Domain score | | |
| NPI-Irritability/Lability Domain score | p = 0.015 | — |
| NPI Total Score | — | — |
| CGIS-Agitation score | p = 0.049 | — |
| ADCS-CGIC-Overall Score | — | — |
| PG IC Score | p = 0.003 | — |
| Zarit Burden Interview (ZB 1) score | — | — |
| Dementia Quality of Life (DEMQOL) | — | — |
| Total score | | |
| DEMQOL-Proxy Total score | p = 0.002 | p = 0.019 |
| CSDD score | — | — |
| ADAS-cog | — | — |

ADAS-cog = Alzheimer's Disease Assessment Scale-cognitive subscale;
ADCS-CGIC-overall = Alzheimer's Disease Cooperative Study-Clinical Global Impression of Change for Overall Clinical Status;
CGIS = Clinical Global Impression of Severity;
CMAI = Cohen-Mansfield Agitation Inventory;
CSDD = Cornell Scale for Depression in Dementia;
mADCS-CGIC = modified Clinical Global Impression of Change scale for Agitation;
mITT = modified intent-to-treat;
NPI = Neuropsychiatric inventory;
PGIC = Patient Global Impression of Change;
SPCD = sequential parallel comparison design
p values are included only for endpoints that were statistically significant at the nominal level determined from the overall weighted SPCD comparison of placebo vs each dose group independently.

### 5.4. Pharmacokinetic and Pharmacodynamic Results

### 5.4.1. d6-DM, Q, and Metabolite Plasma Concentrations

At Day 43 (Visit 4; Week 6) and Day 85 (Visit 6; Week 12), patients had a blood sample collected between 0 and 3 hours after the morning dose of study medication for analysis of plasma levels of d6-DM, d6-DM metabolites, and Q. The time when the patient was administered the dose of study medication and the time of the blood draw were recorded. Plasma samples were separated by centrifugation and frozen at −20° C. until assayed at the analytical unit.

Plasma concentration data for d6-DM, d3-dextrorphan (d3-DX), d3-3-methoxymorphinan (d3-3-MM), and Q are summarized in Table 39 by metabolite, CYP2D6 metabolizer group, treatment group, and visit. There were no notable differences between Week 6 and Week 12 for any analyte at any dose level for all patients combined or in any metabolizer group. Plasma concentrations as shown by mean and median values were generally higher for d6-DM, d3-3-MM, and d3-DX at the higher d6-DM dose, and Q values were similar at both d6-DM doses.

TABLE 39
Plasma Concentrations of d6-Dextromethorphan, d3-Dextrorphan, d3-3-Methoxymorphinan,
and Quinidine by Metabolizer Group, Treatment Group, and Week

| | | | | | | |
|---|---|---|---|---|---|---|
| Analyte | | Poor | Intermediate | Extensive | Ultra-Rapid | |
| (unit) | | Metabolizer | Metabolizer | Metabolizer | Metabolizer | All |
| Treatment | | (PM) | (IM) | (EM) | (UM) | Patients |
| Visit | Statistic | (N = 19) | (N = 91) | (N = 172) | (N = 16) | (N = 298) |

d6-Dextromethorphan (μg/L)
AVP-786-18 (N = 87)

| Week 6 | n | 4 | | 23 | | 53 | | 3 | | 83 | |
|---|---|---|---|---|---|---|---|---|---|---|---|
| | Mean (SD) | 49.21 | (29.769) | 59.35 | (41.627) | 23.68 | (21.842) | 1.79 | (1.076) | 34.00 | (33.138) |
| | Median | 58.90 | | 56.60 | | 19.50 | | 1.94 | | 29.00 | |
| | Min, Max | 7.5, 71.5 | | 0.0, 147.0 | | 0.0, 107.0 | | 0.6, 2.8 | | 0.0, 147.0 | |
| Week 12 | n | 4 | | 21 | | 53 | | 3 | | 81 | |
| | Mean (SD) | 43.80 | (51.925) | 63.96 | (39.539) | 22.03 | (24.565) | 5.82 | (5.148) | 33.38 | (35.350) |

| | | | | | |
|---|---|---|---|---|---|
| Median | 36.60 | 59.90 | 19.10 | 7.70 | 23.00 |
| Min, Max | 0.0, 102.0 | 0.0, 144.0 | 0.0, 130.0 | 0.0, 9.8 | 0.0, 144.0 |

AVP-786-28 (N = 95)

| Week 6 | n | 9 | | 32 | | 42 | | 6 | | 89 | |
|---|---|---|---|---|---|---|---|---|---|---|---|
| | Mean (SD) | 133.59 | (70.309) | 92.15 | (66.005) | 48.27 | (33.362) | 7.69 | (10.981) | 69.94 | (59.929) |
| | Median | 158.00 | | 80.80 | | 50.05 | | 1.83 | | 61.80 | |
| | Min, Max | 9.6, 237.0 | | 0.0, 243.0 | | 0.0, 124.0 | | 0.0, 25.8 | | 0.0, 243.0 | |
| Week 12 | n | 8 | | 30 | | 41 | | 5 | | 84 | |
| | Mean (SD) | 128.09 | (74.239) | 76.00 | (60.743) | 51.57 | (39.708) | 11.37 | (17.918) | 65.19 | (56.737) |

| | | | | | |
|---|---|---|---|---|---|
| Median | 141.50 | 71.20 | 50.00 | 0.57 | 54.05 |
| Min, Max | 0.0, 251.0 | 0.0, 219.0 | 0.0, 149.0 | 0.0, 41.3 | 0.0, 251.0 |

Placebo/AVP-786-18 (N = 65)

| Week 12 | n | 5 | | 21 | | 30 | | 5 | | 61 | |
|---|---|---|---|---|---|---|---|---|---|---|---|
| | Mean (SD) | 91.98 | (61.033) | 53.39 | (42.988) | 38.33 | (36.845) | 11.01 | (11.906) | 45.67 | (43.088) |

| | | | | | |
|---|---|---|---|---|---|
| Median | 78.30 | 43.70 | 30.95 | 8.58 | 31.80 |
| Min, Max | 3.8, 151.0 | 0.0, 129.0 | 0.0, 149.0 | 0.0, 30.0 | 0.0, 151.0 |

Placebo/AVP-786-28 (N = 66)

| Week 12 | n | 1 | | 15 | | 37 | | 3 | | 56 | |
|---|---|---|---|---|---|---|---|---|---|---|---|
| | Mean (SD) | 257.00 | (—) | 63.68 | (54.252) | 45.79 | (41.209) | 0.63 | (1.085) | 51.93 | (53.216) |

| | | | | | |
|---|---|---|---|---|---|
| Median | 257.00 | 64.00 | 49.20 | 0.00 | 46.30 |
| Min, Max | 257.0, 257.0 | 0.0, 148.0 | 0.0, 135.0 | 0.0, 1.9 | 0.0, 257.0 |

d3-3-Methoxymorphinan (μg/L)
AVP-786-18 (N = 87)

| Week 6 | n | 4 | | 23 | | 53 | | 3 | | 83 | |
|---|---|---|---|---|---|---|---|---|---|---|---|
| | Mean (SD) | 35.80 | (41.508) | 38.04 | (38.185) | 9.43 | (10.288) | 0.21 | (0.357) | 18.30 | (26.616) |
| | Median | 26.15 | | 26.80 | | 6.93 | | 0.00 | | 9.08 | |
| | Min, Max | 1.1, 89.8 | | 0.0, 139.0 | | 0.0, 50.4 | | 0.0, 0.6 | | 0.0, 139.0 | |
| Week 12 | n | 4 | | 21 | | 53 | | 3 | | 81 | |
| | Mean (SD) | 36.45 | (46.849) | 42.51 | (40.120) | 9.45 | (11.148) | 0.87 | (0.821) | 19.04 | (28.258) |

| | | | | | |
|---|---|---|---|---|---|
| Median | 23.85 | 31.50 | 6.37 | 0.99 | 8.91 |
| Min, Max | 0.0, 98.1 | 0.0, 144.0 | 0.0, 52.7 | 0.0, 1.6 | 0.0, 144.0 |

AVP-786-28 (N = 95)

| Week 6 | n | 9 | | 32 | | 42 | | 6 | | 89 | |
|---|---|---|---|---|---|---|---|---|---|---|---|
| | Mean (SD) | 115.61 | (69.054) | 57.57 | (47.336) | 21.74 | (19.590) | 3.11 | (5.651) | 42.86 | (48.416) |
| | Median | 106.00 | | 44.70 | | 19.20 | | 0.00 | | 29.20 | |
| | Min, Max | 1.8, 241.0 | | 0.0, 164.0 | | 0.0, 80.1 | | 0.0, 14.0 | | 0.0, 241.0 | |
| Week 12 | n | 7 | | 30 | | 41 | | 5 | | 83 | |
| | Mean (SD) | 132.19 | (88.596) | 47.46 | (46.267) | 22.55 | (22.247) | 3.75 | (6.410) | 39.67 | (50.527) |

| | | | | | |
|---|---|---|---|---|---|
| Median | 106.00 | 37.00 | 17.80 | 0.00 | 23.20 |
| Min, Max | 0.6, 232.0 | 0.0, 166.0 | 0.0, 95.6 | 0.0, 14.8 | 0.0, 232.0 |

Placebo/AVP-786-18 (N = 65)

| Week 12 | n | 5 | | 21 | | 30 | | 5 | | 61 | |
|---|---|---|---|---|---|---|---|---|---|---|---|
| | Mean (SD) | 82.82 | (92.752) | 38.17 | (36.833) | 17.80 | (21.618) | 2.21 | (2.077) | 28.87 | (40.562) |

| | | | | | |
|---|---|---|---|---|---|
| Median | 41.80 | 29.80 | 9.68 | 1.90 | 10.20 |
| Min, Max | 0.7, 236.0 | 0.0, 101.0 | 0.0, 100.0 | 0.0, 5.6 | 0.0, 236.0 |

Placebo/AVP-786-28 (N = 66)

| Week 12 | n | 1 | | 15 | | 38 | | 3 | | 57 | |
|---|---|---|---|---|---|---|---|---|---|---|---|
| | Mean (SD) | 233.00 | (—) | 41.02 | (44.265) | 21.25 | (21.799) | 0.22 | (0.374) | 29.06 | (40.875) |

| | | | | | |
|---|---|---|---|---|---|
| Median | 233.00 | 26.90 | 16.30 | 0.00 | 14.50 |
| Min, Max | 233.0, 233.0 | 0.0, 141.0 | 0.0, 70.1 | 0.0, 0.6 | 0.0, 233.0 |

d3-Dextrorphan (μg/L)
AVP-786-18 (N = 87)

| Week 6 | n | 4 | | 23 | | 53 | | 2 | | 82 | |
|---|---|---|---|---|---|---|---|---|---|---|---|
| | Mean (SD) | 6.50 | (3.783) | 73.80 | (41.821) | 95.19 | (58.225) | 30.01 | (33.651) | 83.27 | (56.084) |
| | Median | 6.24 | | 79.00 | | 98.30 | | 30.01 | | 88.90 | |
| | Min, Max | 2.6, 10.9 | | 0.0, 147.0 | | 0.0, 226.0 | | 6.2, 53.8 | | 0.0, 226.0 | |
| Week 12 | n | 4 | | 21 | | 52 | | 3 | | 80 | |
| | Mean (SD) | 5.11 | (6.058) | 73.60 | (41.221) | 85.58 | (60.735) | 79.80 | (94.058) | 78.19 | (57.870) |

| | | | | | |
|---|---|---|---|---|---|
| Median | 4.27 | 74.20 | 91.80 | 41.30 | 86.80 |
| Min, Max | 0.0, 11.9 | 0.0, 149.0 | 0.0, 261.0 | 11.1, 187.0 | 0.0, 261.0 |

AVP-786-28 (N = 95)

| Week 6 | n | 9 | | 32 | | 42 | | 6 | | 89 | |
|---|---|---|---|---|---|---|---|---|---|---|---|
| | Mean (SD) | 18.96 | (9.169) | 137.21 | (79.734) | 179.89 | (105.229) | 100.98 | (129.001) | 142.95 | (103.477) |
| | Median | 17.10 | | 133.00 | | 172.00 | | 49.95 | | 136.00 | |
| | Min, Max | 2.7, 34.9 | | 0.0, 408.0 | | 0.0, 421.0 | | 0.0, 326.0 | | 0.0, 421.0 | |
| Week 12 | n | 8 | | 29 | | 41 | | 5 | | 83 | |
| | Mean (SD) | 19.69 | (11.719) | 128.08 | (74.718) | 184.34 | (113.092) | 131.06 | (159.510) | 145.60 | (108.705) |

| | | | | | |
|---|---|---|---|---|---|
| Median | 18.60 | 136.00 | 209.00 | 55.90 | 147.00 |
| Min, Max | 0.0, 37.3 | 0.0, 259.0 | 0.0, 422.0 | 0.0, 388.0 | 0.0, 422.0 |

Placebo/AVP-786-18 (N = 65)

| Week 12 | n | 5 | | 21 | | 30 | | 5 | | 61 | |
|---|---|---|---|---|---|---|---|---|---|---|---|
| | Mean (SD) | 12.51 | (10.475) | 79.55 | (53.075) | 116.85 | (83.000) | 132.22 | (118.907) | 96.72 | (78.870) |

| | | | | | |
|---|---|---|---|---|---|
| Median | 11.80 | 85.90 | 120.50 | 118.00 | 87.20 |
| Min, Max | 2.4, 29.7 | 0.0, 214.0 | 0.0, 331.0 | 0.0, 271.0 | 0.0, 331.0 |

d3-Dextrorphan (continued)
Placebo/AVP-786-28 (N = 66)

| Week 12 | n | 1 | | 15 | | 36 | | 2 | | 54 | |
|---|---|---|---|---|---|---|---|---|---|---|---|
| | Mean (SD) | 29.80 | (—) | 110.80 | (81.077) | 136.96 | (112.696) | 141.33 | (186.217) | 127.87 | (105.378) |

| | | | | | |
|---|---|---|---|---|---|
| Median | 29.80 | 104.00 | 122.50 | 141.33 | 113.00 |
| Min, Max | 29.8, 29.8 | 0.0, 317.0 | 0.0, 436.0 | 9.7, 273.0 | 0.0, 436.0 |

Quinidine (μg/L)
AVP-786-18 (N = 87)

| Week 6 | n | 4 | | 23 | | 53 | | 3 | | 83 | |
|---|---|---|---|---|---|---|---|---|---|---|---|
| | Mean (SD) | 18.77 | (12.196) | 27.90 | (17.239) | 18.10 | (13.546) | 11.39 | (8.397) | 20.61 | (15.017) |
| | Median | 18.70 | | 23.50 | | 16.30 | | 12.30 | | 18.30 | |
| | Min, Max | 4.9, 32.8 | | 0.0, 62.1 | | 0.0, 61.6 | | 2.6, 19.3 | | 0.0, 62.1 | |
| Week 12 | n | 4 | | 20 | | 53 | | 3 | | 80 | |
| | Mean (SD) | 7.23 | (9.036) | 26.54 | (16.182) | 17.74 | (13.519) | 15.50 | (13.579) | 19.33 | (14.625) |

| | | | | | |
|---|---|---|---|---|---|
| Median | 5.10 | 25.05 | 17.20 | 21.20 | 19.10 |
| Min, Max | 0.0, 18.7 | 0.0, 59.2 | 0.0, 52.6 | 0.0, 25.3 | 0.0, 59.2 |

AVP-786-28 (N = 95)

| Week 6 | n | 8 | | 31 | | 42 | | 6 | | 87 | |
|---|---|---|---|---|---|---|---|---|---|---|---|
| | Mean (SD) | 22.30 | (17.677) | 25.24 | (15.367) | 23.34 | (14.765) | 7.23 | (10.130) | 22.81 | (15.393) |
| | Median | 17.45 | | 24.40 | | 19.70 | | 3.73 | | 19.70 | |
| | Min, Max | 2.5, 61.3 | | 0.0, 58.8 | | 0.0, 62.7 | | 0.0, 25.9 | | 0.0, 62.7 | |
| Week 12 | n | 8 | | 28 | | 41 | | 5 | | 82 | |
| | Mean (SD) | 18.98 | (11.590) | 24.56 | (14.147) | 25.25 | (16.036) | 8.37 | (11.238) | 23.37 | (15.153) |

| | | | | | |
|---|---|---|---|---|---|
| Median | 20.15 | 27.60 | 22.60 | 4.96 | 23.05 |
| Min, Max | 0.0, 36.4 | 0.0, 55.7 | 0.0, 61.3 | 0.0, 27.9 | 0.0, 61.3 |

Placebo/AVP-786-18 (N = 65)

| Week 12 | n | 5 | | 21 | | 30 | | 5 | | 61 | |
|---|---|---|---|---|---|---|---|---|---|---|---|
| | Mean (SD) | 24.26 | (11.175) | 22.11 | (15.241) | 22.81 | (16.277) | 13.49 | (13.157) | 21.92 | (15.236) |

| | | | | | |
|---|---|---|---|---|---|
| Median | 22.10 | 21.90 | 19.40 | 8.60 | 20.20 |
| Min, Max | 15.1, 43.4 | 0.0, 61.4 | 0.0, 61.5 | 0.0, 34.3 | 0.0, 61.5 |

Placebo/AVP-786-28 (N = 66)

| Week 12 | n | 1 | | 15 | | 37 | | 3 | | 56 | |
|---|---|---|---|---|---|---|---|---|---|---|---|
| | Mean (SD) | 14.60 | (—) | 20.90 | (15.115) | 22.37 | (19.095) | 4.07 | (7.044) | 20.85 | (17.783) |

| | | | | | |
|---|---|---|---|---|---|
| Median | 14.60 | 17.90 | 18.60 | 0.00 | 17.70 |
| Min, Max | 14.6, 14.6 | 0.0, 51.0 | 0.0, 66.6 | 0.0, 12.2 | 0.0, 66.6 |

Max = maximum;
Min = minimum;
SD = standard deviation
Note:
N represents the number of patients who were assigned a metabolizer group; patients who were not assigned a metabolizer group are excluded.
Placebo/AVP-786-18 and Placebo/AVP-786-28 represent patients who were randomized to placebo during Stage 1 and rerandomized to AVP-786 during Stage 2.
Week 12 includes Early Termination visits.

### 5.5. Pharmacokinetic Summary and Discussion

There did not appear to be any significant changes in d6-DM, d3-DX, d3-3-MM, and Q plasma concentrations from Week 6 to Week 12. Exposures to d6-DM and metabolites increased with the increase in d6-DM dose in AVP-786.

### 6. Safety Evaluation

The safety of AVP-786 was assessed by TEAEs, clinical laboratory tests, vital signs, ECGs, and validated instruments that assessed cognition (MMSE), somnolence/sedation (ESS), walking ability (TUG), and suicide risk (S-STS). In addition, TEAEs that may be of particular interest to the AVP-786 program are summarized (fall, sinus bradycardia, rash, thrombocytopenia, and serotonin syndrome).

Safety data are summarized using Safety Population Part 1 and Safety Population Part 2. In Safety Population Part 1, safety data are summarized based on the treatment the patient received in each stage of the study and includes Safety Groups 1 to 5 below. Placebo, AVP-786-28, and AVP-786-18 treatment groups summarize the safety information for the 12-week Parallel Group, who received 12 weeks of treatment exposure. In Safety Population Part 2, safety data are summarized based on the treatment the patient received at any time during the study and includes Safety Groups 6 to 8 below, as well as all patients. All Placebo, All AVP-786-28, and All AVP-786-18 treatment groups summarize the safety information for their corresponding treatment group under 6 weeks or 12 weeks of treatment exposure in either Stage 1, Stage 2, or both. The summary of safety data is primarily based on Safety Population Part 2.
1. Placebo: patients who received placebo for the entire duration of the study (Treatment Segments D and G from the SPCD schematic, FIG. 1; N=63) including data from Treatment Segment A during Stage 1. Note that patients randomized to placebo/AVP-786 but dropped out in Stage 1 are not included in this population. Instead, their data are summarized under the corresponding placebo/AVP-786 treatment group.
2. AVP-786-28: patients who received AVP-786-28 for the entire duration of the study (B and J in FIG. 1; N=97).
3. AVP-786-18: patients who received AVP-786-18 for the entire duration of the study (C and K in FIG. 1; N=95).
4. Placebo/AVP-786-28: patients who received placebo during Stage 1 and AVP-786-28 during Stage 2 (E and H in FIG. 1), including those who received placebo and dropped out in Stage 1. This group is further divided into data that occurred on placebo (Stage 1; N=66) and data that occurred on AVP-786-28 (Stage 2; N=62).
5. Placebo/AVP-786-18: patients who received placebo during Stage 1 and AVP-786-18 during Stage 2 (F and I in FIG. 1) including those who received placebo and dropped out in Stage 1. This group is further divided into data that occurred on placebo (Stage 1; N=65) and data that occurred on AVP-786-18 (Stage 2; N=61).
6. All Placebo: patients who received placebo at any time during the study, including all patients in Segment A during Stage 1 and all patients in Segments D and G during Stage 2 (FIG. 1; N=194). For patients who received both placebo and active treatment, only data from the placebo treatment period are included in the All Placebo group.
7. All AVP-786-28: patients who received AVP-786-28 at any time during the study, including all patients in Segment B during Stage 1 and all patients in Segments J, E, and H during Stage 2 (FIG. 1; N=159). For patients who received both placebo and AVP-786-28 treatment, only data from the AVP-786-28 treatment period are included in the All AVP-786-28 group.
8. All AVP-786-18: patients who received AVP-786-18 at any time during the study, including all patients in Segment C during Stage 1 and all patients in Segments K, F, and I during Stage 2 (FIG. 1; N=156). For patients who received both placebo and AVP-786-18 treatment, only data from the AVP-786-18 treatment period are included in the All AVP-786-18 group.

Safety data are also summarized for all patients combined (N=386).

### 6.1. Extent of Exposure

Exposure to study drug is summarized in Table 40 for Safety Groups 1 to 5 (Safety Groups 6 to 8 were not analyzed for exposure).

For the 12-week Parallel Group, mean (SD) duration of exposure was 80.7 (16.79), 79.4 (20.27), and 83.4 (7.89) days for patients who received placebo, AVP-786-18, and AVP-786-28, respectively.

For patients who received placebo in Stage 1 and were rerandomized to AVP-786 in Stage 2, the mean exposures (SD) to placebo in Stage 1 were 40.6 (7.75) and 40.4 (9.23) for the placebo/AVP-786-18 and placebo/AVP-786-28 groups, respectively. Mean exposures (SD) to AVP-786 in Stage 2 were 43.1 (6.86) and 40.7 (10.02) days for the placebo/AVP-786-18 and placebo/AVP-786-28 groups, respectively.

TABLE 40
Duration of Exposure (Safety Population)

| | | | | Placebo/AVP-786-18 | | Placebo/AVP-786-28 | |
|---|---|---|---|---|---|---|---|
| | Placebo | AVP-786-18 | AVP-786-28 | Placebo | AVP-786-18 | Placebo | AVP-786-28 |
| | (N = 63) | (N = 95) | (N = 97) | (N = 65) | (N = 61) | (N = 66) | (N = 62) |
| Duration of Exposure (days) | | | | | | | |
| n | 63 | 95 | 97 | 65 | 61 | 66 | 62 |
| Mean (SD) | 80.7 (16.79) | 79.4 (20.27) | 83.4 (7.89) | 40.6 (7.75) | 43.1 (6.86) | 40.4 (9.23) | 40.7 (10.02) |
| Median | 85.0 | 85.0 | 85.0 | 42.0 | 43.0 | 42.0 | 43.0 |
| Min, Max | 1, 92 | 1, 100 | 36, 99 | 0, 52 | 10, 59 | 0, 58 | 1, 63 |
| Number of Patient-Days | 5604 | 8055 | 8430 | 2808 | 2803 | 2798 | 2649 |
| Number of Patient-Years | 15.34 | 22.05 | 23.08 | 7.69 | 7.67 | 7.66 | 7.25 |

max = maximum;
min = minimum;
SD = standard deviation
Note:
Denominators are the number of patients who had exposure data.
Number of patient-years = summation over all patients' exposure in days divided by 365.25.

### 6.2. Adverse Events

TEAEs were collected at each visit after the Screening Visit and until 30 days after the last dose of study drug. TEAEs were defined as those AEs that began or worsened on or after the first dose date and before the last dose date+30 days.

### 6.2.1. Brief Summary of Adverse Events

Of the 386 patients in the Safety Population Part 2, 211 patients (54.7%) experienced a total of 571 TEAEs (Table 42). Patients in the All AVP-786-18 group had a higher incidence of TEAEs compared with the All Placebo group (51.3% and 43.8%, respectively); however, the incidence was similar between the All AVP-786-28 and All Placebo groups (45.3% and 43.8%, respectively). Overall, 17.6% of TEAEs were considered related to study drug by the Investigator, with a higher incidence in the All AVP-786-18 and All AVP-786-28 groups (17.3% and 14.5%, respectively) compared with the All Placebo group (10.8%).

Overall, the incidence of SAEs (7.8%), discontinuations due to TEAEs (5.2%), and deaths (1.3%) were low. Consistent with patterns noted above, patients in the All AVP-786-18 group had a higher incidence of SAEs and discontinuations due to TEAEs (10.9% and 5.8%, respectively) compared with the All Placebo group (3.1% and 3.1%, respectively); however, the incidences of these events in the All AVP-786-28 group (4.4% and 3.1%, respectively) were similar to those in the All Placebo group. A total of 5 (1.3%) patients died; 3 in the All AVP-786-18 group, 1 in the All AVP-786-28 group, and 1 in the All Placebo group. None of the deaths were considered related to study drug by the Investigator.

For the 12-week Parallel Group, a similar pattern was observed, where TEAEs, drug-related TEAEs, SAEs, and discontinuation due to TEAEs were experienced in the highest percentage of patients in the AVP-786-18 group (Table 41).

TABLE 41
Overview of Treatment-emergent Adverse Events (Safety Population, Part 1)

| | | | | Placebo/AVP-786-18 | | Placebo/AVP-786-28 | |
|---|---|---|---|---|---|---|---|
| | Placebo | AVP-786-18 | AVP-786-28 | Placebo | AVP-786-18 | Placebo | AVP-786-28 |
| Parameter | (N = 63) | (N = 95) | (N = 97) | (N = 65) | (N = 61) | (N = 66) | (N = 62) |
| Total number of TEAEs | 71 | 152 | 120 | 55 | 74 | 53 | 46 |
| Patients with at least one n (%) | | | | | | | |
| TEAE | 32 (50.8) | 55 (57.9) | 47 (48.5) | 28 (43.1) | 25 (41.0) | 25 (37.9) | 25 (40.3) |
| Drag-related TEAE | 6 (9.5) | 21 (22.1) | 15 (15.5) | 9 (13.8) | 6 (9.8) | 6 (9.1) | 8 (12.9) |
| Non-serious TEAE | 32 (50.8) | 53 (55.8) | 47 (48.5) | 28 (43.1) | 25 (41.0) | 24 (36.4) | 24 (38.7) |
| SAE | 2 (3.2) | 9 (9.5) | 4 (4.1) | 1 (1.5) | 8 (13.1) | 3 (4.5) | 3 (4.8) |
| Drug-related SAE | 0 | 0 | 1 (1.0) | 0 | 0 | 0 | 1 (1.6) |
| Patients discontinued treatment n (%) | | | | | | | |
| Due to TEAE | 3 (4.8) | 7 (7.4) | 1 (1.0) | 1 (1.5) | 2 (3.3) | 2 (3.0) | 4 (6.5) |
| Due to drug-related TEAE | 1 (1.6) | 5 (5.3) | 1 (1.0) | 0 | 0 | 1 (1.5) | 3 (4.8) |
| Due to SAE | 1 (1.6) | 4 (4.2) | 0 | 1 (1.5) | 2 (3.3) | 1 (1.5) | 1 (1.6) |
| Due to drug-related SAE | 0 | 0 | 0 | 0 | 0 | 0 | 1 (1.6) |
| Deaths n (%) | | | | | | | |
| Patients who died | 1 (1.6) | 1 (1.1) | 0 | 0 | 2 (3.3) | 0 | 1 (1.6) |
| Patients who died due to TEAE | 1 (1.6) | 1 (1.1) | 0 | 0 | 2 (3.3) | 0 | 1 (1.6) |
| Patients who died due to drug-related TEAE | 0 | 0 | 0 | 0 | 0 | 0 | 0 |

AE = adverse event;
SAE = serious adverse event;
TEAE = treatment-emergent adverse event
Note:
TEAEs are summarized. A TEAE is defined as an AE where: first dose date ≤ AE start date ≤ date of last dose + 30.
A drug-related event is defined as an event assessed as Possibly Related or Related by the Investigator or where relationship records are missing.
For patients who were randomized to placebo then rerandomized to AVP-786, TEAEs will be counted based on the treatment they were on when the AE occurred.

TABLE 42
Overview of Treatment-emergent Adverse Events (Safety Population, Part 2)

| | All Placebo | | All AVP-786-18 | | All AVP-786-28 | | All Patients | |
|---|---|---|---|---|---|---|---|---|
| Parameter | (N = 194) | | (N = 156) | | (N = 159) | | (N = 386) | |
| Total number of TEAEs | 179 | | 226 | | 166 | | 571 | |
| Patients with at least one n (%) | 85 | (43.8) | 80 | (51.3) | 72 | (45.3) | 211 | (54.7) |
| TEAE | | | | | | | | |
| Drug-related TEAE | 21 | (10.8) | 27 | (17.3) | 23 | (14.5) | 68 | (17.6) |
| Non-serious TEAE | 84 | (43.3) | 78 | (50.0) | 71 | (44.7) | 207 | (53.6) |
| SAE | 6 | (3.1) | 17 | (10.9) | 7 | (4.4) | 30 | (7.8) |
| Drug-related SAE | 0 | | 0 | | 2 | (1.3) | 2 | (0.5) |
| Patients discontinued treatment | 6 | (3.1) | 9 | (5.8) | 5 | (3.1) | 20 | (5.2) |
| n (%) Due to TEAE | | | | | | | | |
| Due to drug-rekited TEAE | 2 | (1.0) | 5 | (3.2) | 4 | (2.5) | 11 | (2.8) |
| Due to SAE | 3 | (1.5) | 6 | (3.8) | 1 | (0.6) | 10 | (2.6) |
| Due to drug-related SAE | 0 | | 0 | | 1 | (0.6) | 1 | (0.3) |
| Deaths n (%) | 1 | (0.5) | 3 | (1.9) | 1 | (0.6) | 5 | (1.3) |
| Patients who died | | | | | | | | |
| Patients who died due to TEAE | 1 | (0.5) | 3 | (1.9) | 1 | (0.6) | 5 | (1.3) |

| | | | | |
|---|---|---|---|---|
| Patients who died due to drug- | 0 | 0 | 0 | 0 |
| related TEAE | | | | |

AE = adverse event;
SAE = serious adverse event;
TEAE = treatment-emergent adverse event
Note:
Only TEAEs are summarized.
A TEAE is defined as an AE where: first dose date ≤ AE start date ≤ date of last dose + 30.
A drug-related event is defined as an event assessed as Possibly Related or Related by the Investigator or where relationship records are missing.
For patients who were randomized to placebo then rerandomized to AVP-786, TEAEs will be counted based on the treatment they were on when the TEAE occurred.

### 6.2.2. Analysis of Adverse Events

### 6.2.2.1. Adverse Events of Greatest Incidence

The incidence of TEAEs was highest in the All AVP-786-18 group (43.8%, 51.3%, and 45.3% in the All Placebo, All AVP-786-18, and All AVP-786-28 groups, respectively). The SOCs with the highest incidence of TEAEs (≥10% patients in any treatment group) in the All Placebo, All AVP-786-18, and All AVP-786-28 groups included Nervous System Disorders (7.7%, 16.7%, and 7.5%, respectively), Gastrointestinal Disorders (8.2%, 14.1%, and 8.8%, respectively), Infections and Infestations (12.9%, 12.2%, and 11.3%, respectively), Investigations (1.5%, 10.9%, and 5.0%, respectively), and Injury, Poisoning and Procedural Complications (8.2%, 10.3%, and 10.1%, respectively).

By PT, the most frequently experienced TEAEs (≥5% patients in any treatment group) were fall (6.2%, 9.6%, and 7.5% in the All Placebo, All AVP-786-18, and All AVP-786-28 groups, respectively), urinary tract infection (5.7%, 7.1%, and 3.1%, respectively), headache (1.5%, 6.4%, and 1.3%, respectively) diarrhoea (2.6%, 6.4%, and 4.4%, respectively), and agitation (5.2%, 2.6%, and 5.0%, respectively), which all occurred at the highest incidence in the All AVP-786-18 group, except for agitation. Agitation was experienced in a higher percentage of patients in the All Placebo and All AVP-786-28 groups compared with the All AVP-786-18 group (Table 43). For the 12-week Parallel Group, a similar pattern was observed, where the most frequently experienced TEAEs by PT, except for agitation and urinary tract infection, occurred at the highest incidence in the AVP-786-18 group.

TABLE 43
Summary of Treatment-emergent Adverse Events Occurring in > 2% of Patients in Any
Treatment Group by System Organ Class and Preferred Term (Safety Population, Part 2)

| | All Placebo | | All AVP-786-18 | | All AVP-786-28 | | All Patients | |
|---|---|---|---|---|---|---|---|---|
| System Organ Class (SOC) | (N = 194) | | (N = 156) | | (N = 159) | | (N = 386) | |
| Preferred Term (PT) | n (%) | | n (%) | | n (%) | | n (%) | |
| Patients with at least one TEAE | 85 | (43.8) | 80 | (51.3) | 72 | (45.3) | 211 | (54.7) |
| Cardiac disorders | 6 | (3.1) | 6 | (3.8) | 8 | (5.0) | 20 | (5.2) |
| Sinus bradycardia | 1 | (0.5) | 4 | (2.6) | 2 | (1.3) | 7 | (1.8) |
| Gastrointestinal disorders | 16 | (8.2) | 22 | (14.1) | 14 | (8.8) | 48 | (12.4) |
| Diarrhoea | 5 | (2.6) | 10 | (6.4) | 7 | (4.4) | 22 | (5.7) |
| Nausea | 4 | (2.1) | 4 | (2.6) | 0 | | 7 | (1.8) |
| Constipation | 4 | (2.1) | 1 | (0.6) | 1 | (0.6) | 6 | (1.6) |
| Vomiting | 0 | | 4 | (2.6) | 0 | | 4 | (1.0) |
| Infections and infestations | 25 | (12.9) | 19 | (12.2) | 18 | (11.3) | 60 | (15.5) |
| Urinary tract infection | 11 | (5.7) | 11 | (7.1) | 5 | (3.1) | 27 | (7.0) |
| Upper respiratory tract infection | 3 | (1.5) | 2 | (1.3) | 4 | (2.5) | 9 | (2.3) |
| Injury, poisoning and procedural | 16 | (8.2) | 16 | (10.3) | 16 | (10.1) | 48 | (12.4) |
| complications | | | | | | | | |
| Fall | 12 | (6.2) | 15 | (9.6) | 12 | (7.5) | 39 | (10.1) |
| Contusion | 4 | (2.1) | 5 | (3.2) | 2 | (1.3) | 11 | (2.8) |
| Metabolism and nutrition disorders | 8 | (4.1) | 13 | (8.3) | 3 | (1.9) | 22 | (5.7) |
| Dehydration | 2 | (1.0) | 4 | (2.6) | 0 | | 6 | (1.6) |
| Nervous system disorders | 15 | (7.7) | 26 | (16.7) | 12 | (7.5) | 52 | (13.5) |
| Somnolence | 7 | (3.6) | 6 | (3.8) | 4 | (2.5) | 16 | (4.1) |
| Dizziness | 5 | (2.6) | 5 | (3.2) | 5 | (3.1) | 15 | (3.9) |
| Headache | 3 | (1.5) | 10 | (6.4) | 2 | (1.3) | 15 | (3.9) |
| Psychiatric disorders | 18 | (9.3) | 6 | (3.8) | 14 | (8.8) | 36 | (9.3) |
| Agitation | 10 | (5.2) | 4 | (2.6) | 8 | (5.0) | 21 | (5.4) |
| Depression | 4 | (2.1) | | | | | | |

Note:
Adverse events are coded using MedDRA version 18.1
if a patient has more than one event that codes to the same MedDRA category, the patient is counted only once in the MedDRA category.
For patients who were randomized to placebo then rerandomized to AVP-786, TEAEs will be counted based on the treatment they were on when the AE occurred.

### 6.2.2.2. Common Adverse Events by Time to Onset, Duration, and Recurrence

Common TEAEs were summarized by time to onset, duration, and recurrence. For these analyses, a common TEAE was defined as a TEAE with an incidence that was ≥3% in the All AVP-786 groups AND was ≥2 times the incidence of the All Placebo group. The only TEAEs that met this definition were diarrhoea (2.6%, 6.4%, and 4.4% in the All Placebo, All AVP-786-18, and All AVP-786-28 groups, respectively) and headache (1.5%, 6.4%, and 1.3%, respectively).

The median time to onset of diarrhoea was 18.0, 25.5, and 20.0 days in the All Placebo, All AVP-786-18, and All AVP-786-28 groups, respectively, and the median time to onset of headache was 3.0, 14.0, and 25.0 days, respectively.

The median duration of diarrhoea was 2.0 days (2.5% of study days), 3.0 days (7.6%), and 11.0 days (13.3%) in the All Placebo, All AVP-786-18, and All AVP-786-28 groups, respectively, and the median duration of headache was 2.0 days (2.4% of study days), 1.5 days (1.8%). and 5.5 days (6.48%), respectively.

Only 1 patient experienced a recurrence of a common TEAE; a patient in the All AVP-786-18 group experienced a recurrent headache.

### 6.2.2.3. Relationship of Adverse Events to Study Drug

Patients in the All AVP-786-18 and All AVP-786-28 groups had a higher incidence of drug-related TEAEs (17.3% and 14.5%, respectively) compared with the All Placebo group (10.8%) (Table 44). By SOC, the most frequently experienced drug-related TEAEs (≥3% patients in any treatment group) in the All Placebo, All AVP-786-18, and All AVP-786-28 groups were Investigations (0.5%, 5.1%, and 1.3%, respectively), Nervous System Disorders (2.6%, 3.8%, and 5.0%, respectively), Gastrointestinal Disorders (3.6%, 3.8%, and 2.5%, respectively), and Cardiac Disorders (0.5%, 2.6%, and 3.8%, respectively).

TABLE 44
Summary of Treatment-emergent Drug-related Adverse Events Experienced
by ≥2 Patients by System Organ Class and Preferred Term (Safety
Population, Part 2)

| | All Placebo | All AVP-786-18 | All AVP-786-28 | All Patients |
|---|---|---|---|---|
| System Organ Class (SOC) | (N = 194) | (N = 156) | (N = 159) | (N = 386) |
| Preferred Term (PT) | n (%) | n (%) | n (%) | n (%) |
| Patients with at least one drug-related TEAE | 21 (10.8) | 27 (17.3) | 23 (14.5) | 68 (17.6) |
| Cardiac disorders | 1 (0.5) | 4 (2.6) | 6 (3.8) | 11 (2.8) |
| Sinus bradycardia | 0 | 2 (1.3) | 1 (0.6) | 3 (0.8) |
| Ventricular extrasystoles | 0 | 0 | 2 (1.3) | 2 (0.5) |
| Bundle branch block left | 0 | 1 (0.6) | 1 (0.6) | 2 (0.5) |
| Gastrointestinal disorders | 7 (3.6) | 6 (3.8) | 4 (2.5) | 17 (4.4) |
| Diarrhoea | 3 (1.5) | 4 (2.6) | 3 (1.9) | 10 (2.6) |
| Nausea | 1 (0.5) | 2 (1.3) | 0 | 3 (0.8) |
| Constipation | 3 (1.5) | 0 | 0 | 3 (0.8) |
| General disorders and administration site | 3 (1.5) | 2 (1.3) | 2 (1.3) | 6 (1.6) |
| conditions | | | | |
| Fatigue | 2 (1.0) | 1 (0.6) | 1 (0.6) | 3 (0.8) |
| Injury, poisoning and procedural | 0 | 4 (2.6) | 2 (1.3) | 6 (1.6) |
| complications | | | | |
| Fall | 0 | 3 (1.9) | 2 (1.3) | 5 (1.3) |
| Investigations | 1 (0.5) | 8 (5.1) | 2 (1.3) | 11 (2.8) |
| Electrocardiogram QT prolonged | 0 | 2 (1.3) | 2 (1.3) | 4 (1.0) |
| Weight increased | 1 (0.5) | 2 (1.3) | 0 | 3 (0.8) |
| Metabolism and nutrition disorders | 2 (1.0) | 2 (1.3) | 1 (0.6) | 5 (1.3) |
| Decreased appetite | 1 (0.5) | 1 (0.6) | 1 (0.6) | 3 (0.8) |
| Nervous system disorders | 5 (2.6) | 6 (3.8) | 8 (5.0) | 19 (4.9) |
| Somnolence | 3 (1.5) | 5 (3.2) | 4 (2.5) | 12 (3.1) |
| Dizziness | 1 (0.5) | 1 (0.6) | 2 (1.3) | 4 (1.0) |
| Psychiatric disorders | 5 (2.6) | 0 | 2 (1.3) | 7 (1.8) |
| Agitation | 3 (1.5) | 0 | 0 | 3 (0.8) |
| Depression | 2 (1.0) | 0 | 0 | 2 (0.5) |

Note:
Adverse events are coded using MedDRA version 18.1.
If a patient has more than one event that codes to the same MedDRA category, the patient is counted only once in the MedDRA category.
For patients who were randomized to placebo then rerandomized to AVP-786, TEAEs will be counted based on the treatment they were on when the AE occurred.
Drug-related is defined as an event assessed as Possibly Related or Related by the Investigator or where relationship records are missing.

### 6.2.2.4. Adverse Events by Severity

Overall, the incidence of severe TEAEs was low during the study (5.2%). Patients in the All AVP-786 groups had a higher incidence of severe TEAEs compared with the All Placebo group (1.5%, 7.7%, and 3.1% in the All Placebo, All AVP-786-18, and All AVP-786-28 groups, respectively). Severe TEAEs experienced by ≥2 patients in any treatment group were fall (0%, 1.3%, and 0% in the All Placebo, All AVP-786-18, and All AVP-786-28 groups, respectively) and syncope (0%, 1.3%, and 0%, respectively).

### 6.2.2.5. Analysis of Treatment-Emergent Adverse Events by Baseline Use of Major CYP2D6 Substrate Beta Blocker Concomitant Medications

There were 38, 33, and 24 patients in the All Placebo, All AVP-786-18, and All AVP-786-28 groups, respectively, who were using beta blockers that are classified as major CYP2D6 substrates at the Baseline Visit. In general, the overall TEAE profile for these patients was consistent with that observed for the full safety analysis population. Overall, 42.1%, 51.5%, and 33.3% of patients in the All Placebo, All AVP-786-18, and All AVP-786-28 groups experienced at least one TEAE. By PT, fall, diarrhoea, and headache were the most frequently experienced TEAEs (10.4%, 6.0%, and 6.0%, respectively) in patients who were using beta blockers that are classified as major CYP2D6 substrates at the Baseline Visit (Table 45).

TABLE 45
Summary of Treatment-emergent Adverse Events Experienced by
≥2 Patients with Baseline Use of Beta Blocker Concomitant Medications that
are Major CYP2D6 Substrates by Preferred Term in Any Treatment Group
(Safety Population, Part 2)

| | All Placebo | All AVP-786-18 | All AVP-786-28 | All Patients |
|---|---|---|---|---|
| System Organ Class (SOC) | (N = 38) | (N = 33) | (N = 24) | (N = 67) |
| Preferred Term (PT) | n (%) | n (%) | n (%) | n (%) |
| Patients with at least one TEAE | 16 (42.1) | 17 (51.5) | 8 (33.3) | 36 (53.7) |
| Fall | 3 (7.9) | 2 (6.1) | 2 (8.3) | 7 (10.4) |
| Diarrhoea | 2 (5.3) | 2 (6.1) | 0 | 4 (6.0) |
| Headache | 1 (2.6) | 3 (9.1) | 0 | 4 (6.0) |
| Upper respiratory tract infection | 0 | 2 (6.1) | 1 (4.2) | 3 (4.5) |
| Agitation | 2 (5.3) | 1 (3.0) | 1 (4.2) | 3 (4.5) |
| Ventricular extrasystoles | 0 | 0 | 2 (8.3) | 2 (3.0) |
| Sinus bradycardia | 0 | 2 (6.1) | 0 | 2 (3.0) |
| Vomiting | 0 | 2 (6.1) | 0 | 2 (3.0) |
| Nausea | 2 (5.3) | 0 | 0 | 2 (3.0) |
| Pain | 1 (2.6) | 1 (3.0) | 0 | 2 (3.0) |
| Nasopharyngitis | 1 (2.6) | 0 | 1 (4.2) | 2 (3.0) |
| Urinary tract infection | 1 (2.6) | 0 | 1 (4.2) | 2 (3.0) |
| Contusion | 1 (2.6) | 0 | 1 (4.2) | 2 (3.0) |
| Hyperkalaemia | 1 (2.6) | 1 (3.0) | 0 | 2 (3.0) |
| Dizziness | 1 (2.6) | 1 (3.0) | 0 | 2 (3.0) |
| Somnolence | 1 (2,6) | 1 (3.0) | 0 | 2 (3.0) |

AE = adverse event;
MedDRA = Medical Dictionary for Regulatory Activities;
TEAE = treatment-emergent adverse event
Note:
Adverse events are coded using MedDRA version 18.1.
If a patient has more than one event that codes to the same MedDRA category, the patient is counted only once in the MedDRA category.
For patients who were randomized to placebo then rerandomized to AVP-786, TEAEs will be counted based on the treatment they were on when the AE occurred.

The number of patients who were using beta blockers at the Baseline Visit that are not classified as major CYP2D6 substrates was low (12, 12, and 9 in the All Placebo, All AVP-786-18, and All AVP-786-28 groups, respectively).

### 6.3. Deaths, Other Serious Adverse Events, and Other Significant Adverse Events

### 6.3.1. Analysis and Discussion of Other Serious Adverse Events, and Other Significant Adverse Events

### 6.3.1.1. Other Serious Adverse Events

Overall, 7.8% of patients experienced at least 1 SAE during the study. Patients in the All AVP-786-18 group had a higher incidence of SAEs compared with the All Placebo group (10.9% and 3.1%, respectively); however, the incidence of SAEs was similar between the All AVP-786-28 and All Placebo groups (4.4% and 3.1%, respectively). Few SAEs were experienced by more than 1 patient (Table 47).

Overall, the SOCs with the highest incidence of SAEs were Infections and Infestations (2.6%) and Injury, Poisoning and Procedural Complications (1.8%). SAEs experienced in the SOC Infections and Infestations were experienced with a similar incidence across the treatment groups (1.5%, 2.6%, and 1.9% in the All Placebo, All AVP-786-18, and All AVP-786-28 groups, respectively). SAEs experienced in the SOC Injury, Poisoning and Procedural Complications were experienced at the highest incidence in the All AVP-786-18 group but occurred with similar incidences in the All Placebo and All AVP-786-28 groups (0%, 3.8%, and 0.6% in the All Placebo, All AVP-786-18, and All AVP-786-28 groups, respectively).

By PT, SAEs experienced by ≥2 patients any treatment group were urinary tract infection (1.0%, 1.3%, and 0.6% in the All Placebo, All AVP-786-18, and All AVP-786-28 groups, respectively), alcohol poisoning (0%, 1.3%, and 0%, respectively), cerebrovascular accident (0%, 1.3%, and 0%, respectively), and atrial fibrillation (1.0%, 0%, and 0%, respectively; Table 47).

Two patients experienced SAEs that were considered drug-related by the Investigator; both occurred during treatment with AVP-786-28: bradycardia Table 47: Summary of Treatment-emergent Serious Adverse Events Experienced by

TABLE 47
Summary of Treatment-emergent Serious Adverse Events Experienced by
≥2 Patients in Any Treatment Group by Preferred Term (Safety Population,
Part 2)

| | All Placebo | All AVP-786-18 | All AVP-786-28 | All Patients |
|---|---|---|---|---|
| | (N = 194) | (N = 156) | (N = 159) | (N = 386) |
| Preferred Term (PT) | n (%) | n (%) | n (%) | n (%) |
| Patients with at least one SAE | 6 (3.1) | 17 (10.9) | 7 (4.4) | 30 (7.8) |
| Urinary tract infection | 2 (1.0) | 2 (1.3) | 1 (0.6) | 5 (1.3) |
| Alcohol poisoning | 0 | 2 (1.3) | 0 | 2 (0.5) |
| Cerebrovascular accident | 0 | 2 (1.3) | 0 | 2 (0.5) |
| Atrial fibrillation | 2 (1.0) | 0 | 0 | 2 (0.5) |

AE = adverse event;
MedDRA = Medical Dictionary for Regulatory Activities;
TEAE = treatment-emergent adverse event
Note:
Adverse events are coded using MedDRA version 18.1.
Only TEAEs are included. A TEAE is defined as an AE where: first dose date ≤ AE start date ≤ date of last dose + 30.
If a patient has more than one event that codes to the same MedDRA category, the patient is counted only once in the MedDRA category.
For patients who were randomized to placebo then rerandomized to AVP-786, SAEs will be counted based on the treatment they were on when the AE occurred.

### 6.3.1.2. Other Significant Adverse Events

### 6.3.1.2.1. Adverse Events Leading to Discontinuation of Treatment

Overall, 5.2% of patients discontinued treatment due to TEAEs. TEAEs led to discontinuation of treatment for 3.1%, 5.8%, and 3.1% patients in the All Placebo, All AVP-786-18, and All AVP-786-28 groups, respectively (Table 48). The TEAEs leading to discontinuation of ≥2 patients in any treatment group were fall (0%, 1.3%, and 1.3% in the All Placebo, All AVP-786-18, and All AVP-786-28 groups, respectively), atrial fibrillation (1.0%, 0.6%, and 0%, respectively), electrocardiogram QT prolonged (0%, 0.6%, and 1.3%, respectively), and agitation (1.0%, 0%, and 0.6%, respectively).

Drug-related TEAEs led to discontinuation of treatment for 1.0%, 3.2%, and 2.5% patients in the All Placebo, All AVP-786-18, and All AVP-786-28 groups, respectively. Drug-related TEAE that led to discontinuation of ≥2 patients in any treatment group were fall (0%4, 0.6%, and 1.3% in the All Placebo, All AVP-786-18, and All AVP-786-28 groups, respectively) and electrocardiogram QT prolonged (0%, 0.6%, and 1.3%, respectively).

TABLE 48
Summary of Treatment-emergent Adverse Events Leading to
Discontinuation by Preferred Term (Safety Population, Part 2)

| | All Placebo | All AVP-786-18 | All AVP-786-28 | All Patients |
|---|---|---|---|---|
| | (N = 194) | (N = 156) | (N = 159) | (N = 386) |
| Preferred Term (PT) | n (%) | n (%) | n (%) | n (%) |
| Patients with at least one TEAE | 6 (3.1) | 9 (5.8) | 5 (3.1) | 20 (5.2) |
| leading to discontinuation | | | | |
| Fall | 0 | 2 (1.3) | 2 (1.3) | 4 (1.0) |
| Atrial fibrillation | 2 (1.0) | 1 (0.6) | 0 | 3 (0.8) |
| Electrocardiogram QT prolonged | 0 | 1 (0.6) | 2 (1.3) | 3 (0.8) |
| Agitation | 2 (1.0) | 0 | 1 (0.6) | 3 (0.8) |
| Decreased appetite | 1 (0.5) | 1 (0.6) | 0 | 2 (0.5) |
| Diarrhoea | 0 | 1 (0.6) | 0 | 1 (0.3) |
| Injury associated with device | 1 (0.5) | 0 | 0 | 1 (0.3) |
| Sepsis | 0 | 1 (0.6) | 0 | 1 (0.3) |
| Urinary tract infection | 1 (0.5) | 0 | 0 | 1 (0.3) |
| Ligament sprain | 0 | 0 | 1 (0.6) | 1 (0.3) |
| Alcohol poisoning | 0 | 1 (0.6) | 0 | 1 (0.3) |
| Laceration | 0 | 1 (0.6) | 0 | 1 (0.3) |
| Rib fracture | 0 | 1 (0.6) | 0 | 1 (0.3) |
| Spinal compression fracture | 0 | 1 (0.6) | 0 | 1 (0.3) |
| White blood cell count increased | 0 | 1 (0.6) | 0 | 1 (0.3) |
| Muscular weakness | 1 (0.5) | 0 | 0 | 1 (0.3) |
| Cerebrovascular accident | 0 | 1 (0.6) | 0 | 1 (0.3) |
| Lethargy | 0 | 1 (0.6) | 0 | 1 (0.3) |
| Somnolence | 0 | 1 (0.6) | 0 | 1 (0.3) |
| Delirium | 0 | 1 (0.6) | 0 | 1 (0.3) |
| Depression | 1 (0.5) | 0 | 0 | 1 (0.3) |
| Haemothorax | 0 | 1 (0.6) | 0 | 1 (0.3) |

AE = adverse event;
MedDRA = Medical Dictionary for Regulatory Activities;
TEAE = treatment-emergent adverse event
Note:
Adverse events are coded using MedDRA version 18.1.
If a patient has more than one event that codes to the same MedDRA category, the patient is counted only once in the MedDRA category.
For patients who were randomized to placebo then rerandomized to AVP-786, AEs will be counted based on the treatment they were on when the AE occurred.

### 6.3.1.2.2. Treatment-Emergent Adverse Events of Interest

### Fall

Fall was the most frequently experienced TEAE across all treatment groups, with 10.1% patients overall reporting at least 1 TEAE of fall (Table 49). Patients in the All AVP-786-18 group had the highest incidence of falls (9.6%) compared with patients in the All Placebo (6.2%) and All AVP-786-28 (7.5%) groups.

Overall, few falls were experienced as SAEs (0.5%), considered related to study drug by the Investigator (1.3%), led to discontinuation of treatment (1.0%), or were experienced as severe in intensity (0.5%). These types of fall events were experienced only in patients treated with AVP-786-18 or AVP-786-28.

Two patients treated with AVP-786-18 (AVP-786-18 group) experienced severe TEAEs of fall, one of which resulted in discontinuation from treatment. One patient treated with AVP-786-28 experienced an SAE of fall of moderate severity that was considered possibly related to study drug; study drug was withdrawn because of the fall. One subject experienced an SAE of fall of moderate severity that was considered unrelated to study drug; study drug was interrupted because of the fall.

TABLE 49
Summary of Treatment-emergent Adverse Events Fall (Safety Population,
Part 2)

| | All Placebo | All AVP-786-18 | All AVP-786-28 | All Patients |
|---|---|---|---|---|
| Parameter | (N = 194) | (N = 156) | (N = 159) | (N = 386) |
| Patients with at least one n (%) | | | | |
| Fall | 12 (6.2) | 15 (9.6) | 12 (7.5) | 39 (10.1) |
| Severe fall | 0 | 2 (1.3) | 0 | 2 (0.5) |
| Drug-related fall | 0 | 3 (1.9) | 2 (1.3) | 5 (1.3) |
| Serious fall | 0 | 1 (0.6) | 1 (0.6) | 2 (0.5) |
| Drag-related serious fall | 0 | 0 | 1 (0.6) | 1 (0.3) |
| Patients discontinued treatment | | | | |
| because of a fall n (%) | | | | |
| Due to TEAE | 0 | 2 (1.3) | 2 (1.3) | 4 (1.0) |

AE = adverse event;
MedDRA = Medical Dictionary for Regulatory Activities;
TEAE = treatment-emergent adverse event
Note:
Only TEAEs are included. A TEAE is defined as an AE where: first dose date ≤ AE start date ≤ date of last dose + 30.
A drug-related event is defined as an event assessed as Possibly Related or Related by the Investigator or where relationship records are missing.
For patients who were randomized to placebo then rerandomized to AVP-786, AEs will be counted based on the treatment they were on when the AE occurred.

### Sinus Bradycardia

Sinus bradycardia events includes TEAEs of sinus bradycardia and bradycardia. Eight patients (2.1%) experienced sinus bradycardia events (Table 50); 1 (0.5%), 4 (2.6%), and 3 (1.9/6) patients in the All Placebo, All AVP-786-18, and All AVP-786-28 groups, respectively. Overall, few events were experienced as SAEs (0.3%) or considered drug-related (1.0%); none of these events resulted in discontinuation of study drug.

All sinus bradycardia events were mild (1.3%) or moderate (0.8%) in severity.

One patient experienced an SAE of bradycardia. On Study Day 84 at the Week 12 visit, an electrocardiogram showed extreme bradycardia with a heart rate of 36 bpm beats per minute. An AE of bradycardia, which was of moderate intensity and considered possibly related to study drug, was experienced on Study Day 84. On Study Day 92 (8 days after the last dose of study drug), the event of bradycardia became serious. On the same day, the patient was hospitalized and a permanent pacemaker was inserted due to a 13 second pause of complete heart block. The outcome of the event was recovered/resolved on Study Day 93.

The other drug-related sinus bradycardia events included a mild event that began on Study Day 85 that resolved the same day, a moderate event that was ongoing at the end of study, and a moderate event on Study Day 43 that recovered/resolved the same day.

TABLE 50
Summary of Treatment-emergent Adverse Events-Sinus Bradycardia
(Safety Population, Part 2)

| | All Placebo | All AVP-786-18 | All AVP-786-28 | All Patients |
|---|---|---|---|---|
| Parameter | (N = 194) | (N = 156) | (N = 159) | (N = 386) |
| Patients with at least one n (%) | | | | |
| Sinus bradycardia event | 1 (0.5) | 4 (2.6) | 3 (1.9) | 8 (2.1) |
| Drag-related sinus bradycardia event | 0 | 2 (1.3) | 2 (1.3) | 4 (1.0) |
| Serious sinus bradycardia event | 0 | 0 | 1 (0.6) | 1 (0.3) |
| Drug-related serious sinus bradycardia | 0 | 0 | 1 (0.6) | 1 (0.3) |
| event | | | | |

AE = adverse event;
MedDRA = Medical Dictionary for Regulatory Activities;
TEAE = treatment-emergent adverse event
Note:
Sinus bradycardia events include sinus bradycardia and bradycardia.
Only TEAEs are included. A TEAE is defined as an AE where: first dose date ≤ AE start date ≤ date of last dose + 30.
A drug-related event is defined as an event assessed as Possibly Related or Related by the Investigator or where relationship records are missing.
For patients who were randomized to placebo then rerandomized to AVP-786, AEs will be counted based on the treatment they were on when the AE occurred.

Rash

Rash events include TEAEs of rash and eczema. A total of 4 rash events were experienced in patients treated with AVP-786; 2 patients each in the AVP-786-18 and AVP-786-28 groups (1.3% and 1.3%, respectively). All events of rash were considered mild in severity, none were reported SAEs, and none led to treatment discontinuation.

Two rash events which occurred in patients in the AVP-786-28 group appeared to be due to fungal infections.

The 2 remaining rash events occurred in patients in the AVP-786-18 group. In one case, the patient experienced a mild, drug-related rash that resolved in 4 days; the dose of study drug was not changed due to the TEAE. In the remaining rash event, the patient experienced a mild rash that resolved in 16 days; the rash was unlikely related to study drug, and the dose of study drug was not changed due to the TEAE.

Thrombocytopenia

One patient treated with AVP-786-18 (placebo/AVP-786-18 group) experienced a TEAE of thrombocytopenia. The event was mild, nonserious, and considered not related to study drug; the dose of study drug was not changed due to the TEAE and the event was ongoing at the end of study. The patient's platelet count had decreased from a Baseline value of 163×10⁹/L (normal range 150-450×10⁹/L) to 143×10⁹/L at Week 6 (start date of TEAE). The patient's lowest platelet count during the TEAE was 136×10⁹/L at Week 12 (end of study 15-AVP-786-301). However, the patient's platelet count did not meet the potentially clinically significant (PCS) criterion for low platelets (≤100×10⁹/L).

For hematology laboratory tests results, a total of 3 patients met PCS criterion for low platelets; 1 patient each while treated with placebo (placebo group), AVP-786-18 (placebo/AVP-786-18 group), and AVP-786-28 (AVP-786-28 group). None of the patients who met PCS criterion for low platelets experienced a TEAE of thrombocytopenia. For the 12-week Parallel group, the percentage of patients with shifts in platelets from normal or high to low were 0%, 4.3%, and 1.1% for the placebo, AVP-786-18, and AVP-786-28 groups, respectively.

Serotonin Syndrome

No patients experienced TEAEs of serotonin syndrome

### 6.4. Clinical Laboratory Evaluation

### 6.4.1. Evaluation of Each Laboratory Parameter

In general, there was no evidence of clinically meaningful changes from Baseline in mean values of chemistry or hematology parameters, or in quantitative measures of urinalysis.

### 6.4.1.1. Laboratory Values Over Time

The proportion of patients with shifts in chemistry and hematology values from either low, normal, or high at Baseline to low, normal, or high at the end of treatment are presented by visit for Safety Groups 1 to 3 (12-week Parallel Group): shifts in laboratory values were not summarized for the All treatment groups because of the complexity of multiple baselines.

### 6.4.1.1.1. Chemistry

Overall, a small percentage of patients had shifts (low, normal, or high, relative to the normal range) from Baseline to end of treatment in chemistry parameters.

In the 12-week Parallel Group, the only chemistry parameters for which ≥10% of patients in any treatment group were both normal at Baseline and high at end of treatment were alkaline phosphatase (8.2%, 9.7%, and 12.5% below for placebo, AVP-786-18, and AVP-786-28 groups, respectively), BUN (11.5%, 6.5%, and 7.3%, respectively), and HbA1c (0%, 6.3%, and 12.1%, respectively; Note that most patients did not have more than 1 HbA1c result for analysis [n=22, 32, and 33, respectively]).

### 6.4.1.1.2. Hematology

Overall, a small percentage of patients had shifts (low, normal, or high, relative to the normal range) from Baseline to end of treatment in hematology parameters. In the 12-week Parallel Group, no hematology parameters met the criterion of >10% of patients in any treatment group with clinically relevant shifts from Baseline to end of treatment. In general, the proportions of patients with shifts were similar across the treatment groups.

### 6.4.1.1.3. Urinalysis

There were no clinically meaningful changes in urinalysis measures.

### 6.4.1.2. Individual Potentially Clinically Significant Abnormalities

Overall, a low percentage of patients met PCS criteria for chemistry or hematology parameters. Table 51 summarizes the parameters where PCS criteria was met for >5% patients in any treatment groups.

The proportion of patients with PCS abnormalities in chemistry or hematology parameters was generally similar among the treatment groups. A higher proportion of patients in the All AVP-786-28 group met the PCS criteria compared with the All Placebo group for elevated creatinine (6.3% vs 1.9%, respectively) and low lymphocytes/leukocytes (8.2% vs 1.3%, respectively). Note that at Baseline 8.3% and 9.8% patients in the All AVP-786-28 and All Placebo groups, respectively, had creatinine greater than the upper limit of normal, and at Baseline 13.5% and 11.5%, respectively, had low lymphocytes/leukocytes.

TABLE 51
Potentially Clinically Significant Postbaseline Chemistry and Hematology Laboratory
Abnormalities in >5% of Patients in Any Group (Safety Population, Part 2)

| | All Placebo | | | All AVP-786-18 | | All AVP-786-28 | | All Patients | |
|---|---|---|---|---|---|---|---|---|---|
| Parameter | (N = 194) | | | (N = 156) | | (N = 159) | | (N = 386) | |
| PCS Criterion | N | n (%) | | N | n (%) | N | n (%) | N | n (%) |
| Serum Chemistry | | | | | | | | | |
| BUN | | | | | | | | | |
| ≥10.71 mmol/L | 159 | 13 | (8.2) | 154 | 14 (9.1) | 158 | 15 (9.5) | 376 | 40 (10.6) |
| Creatinine | | | | | | | | | |
| >132.6 μmol/L | 159 | 3 | (1.9) | 154 | 5 (3.2) | 158 | 10 (6.3) | 376 | 16 (4.3) |
| GGT | | | | | | | | | |
| ≥60 U/L | 159 | 5 | (3.1) | 154 | 7 (4.5) | 158 | 10 (6.3) | 376 | 19 (5.1) |
| Glucose | | | | | | | | | |
| ≥11.1 mmol/L | 159 | 14 | (8.8) | 154 | 8 (5.2) | 158 | 10 (6.3) | 376 | 25 (6.6) |
| Triglycerides | | | | | | | | | |
| >3.39 mmol/L | 159 | 15 | (9.4) | 154 | 22 (14.3) | 158 | 14 (8.9) | 376 | 48 (12.8) |
| Hematology | | | | | | | | | |
| Lymphocytes/Leukocytes (%) | | | | | | | | | |
| ≤10% | 159 | 2 | (1.3) | 153 | 2 (1.3) | 158 | 13 (8.2) | 375 | 16 (4.3) |

BUN = blood urea nitrogen;
GGT = gamma-glutamyl transferase;
PCS = potentially clinically significant
Note:
For patients who were randomized to placebo then rerandomized to AVP-786, patients were counted based on the treatment they were on when the PCS criterion was met.

### 6.5. Vital Signs, Physical Examination Findings, and Other Observations Related to Safety

### 6.5.1. Electrocardiograms

In general, there was no evidence of a clinically meaningful mean change in any ECG parameter for any treatment group between or within visits. Mean and median changes for QTcF were within ±2% for each visit for each group and ±6% within visit. The mean (SD) changes from Baseline at Week 12 were −2.1 (14.5), 6.6 (15.3), and 4.4 (15.9) msec for the placebo, AVP-786-18, and AVP-786-28 groups, respectively. The mean changes from predose to postdose on Day 1 were 2.7 (14.1), 4.5 (13.7), and 1.1 (12.6) msec, respectively.

A low number of patients met the PCS criteria of QTcF>500 msec (males and females combined) or increase in QTcF>60 msec compared to Baseline in the All Placebo, All AVP-786-18, and All AVP-786-28 groups (0, 2 [1.3%], and 3 [1.9%], respectively; 1 [0.5%], 2 [1.3%], and 2 [1.3%], respectively; males and females combined) (Table 52).

The highest incidence of TEAEs that were in the SOC Cardiac Disorders or SOC Investigations related to cardiac function were sinus bradycardia (0.5%, 2.6%, and 1.3% in the All Placebo, All AVP-786-18, and All AVP-786-28 groups, respectively; Table 53) and electrocardiogram QT prolonged (0%, 1.9%, and 1.9%, respectively; Table 53).

TABLE 52
Overall Potentially Clinically Significant Postbaseline ECG Abnormalities (Safety Population, Part 2)

| | | All Placebo | | All AVP-786-18 | | All AVP-786-28 | | All Patients | |
|---|---|---|---|---|---|---|---|---|---|
| | | (N = 186) | | (N = 156) | | (N = 159) | | (N = 386) | |
| Parameter | PCS Criterion | N | n (%) | N | n (%) | N | n (%) | N | n (%) |
| QTcF Value (males) | >450 to ≤480 msec | 78 | 5 (6.4) | 72 | 10 (13.9) | 70 | 4 (5.7) | 168 | 18 (10.7) |
| | >480 to ≤500 msec | 78 | 1 (1.3) | 72 | 1 (1.4) | 70 | 1 (1.4) | 168 | 3 (1.8) |
| | >500 msec | 78 | 0 | 72 | 2 (2.8) | 70 | 1 (1.4) | 168 | 3 (1.8) |
| QTcF Value (females) | >470 to ≤485 msec | 109 | 1 (0.9) | 82 | 1 (1.2) | 85 | 3 (3.5) | 209 | 5 (2.4) |
| | >485 to ≤500 msec | 109 | 1 (0.9) | 82 | 0 | 85 | 2 (2.4) | 209 | 3 (1.4) |
| | >500 msec | 109 | 0 | 82 | 0 | 85 | 2 (2.4) | 209 | 2 (1.0) |
| QTcF Change from Baseline (male and females) | >30 msec | 187 | 11 (5.9) | 154 | 25 (16.2) | 154 | 19 (12.3) | 376 | 52 (13.8) |
| | >60 msec | 187 | 1 (0.5) | 154 | 2 (1.3) | 154 | 2 (1.3) | 376 | 5 (1.3) |

ECG = electrocardiogram;
PCS = potentially clinically significant;
QTcF = QTc by Fridericia's formula
Note:
For patients who were randomized to placebo then rerandomized to AVP-786, patients were counted based on the treatment they were on when the PCS criterion was met. For patients who were randomized to placebo then rerandomized to AVP-786, Baseline for the placebo portion is the last assessment prior to first dose and Baseline for the AVP-786 portion is the last assessment prior to rerandomization.

TABLE 53
Electrocardiogram Abnormalities Experienced as Adverse Events (Safety
Population, Part 2)

| | All Placebo | All AVP-786-18 | All AVP-786-28 | All Patients |
|---|---|---|---|---|
| System Organ Class (SOC) | (N = 194) | (N = 156) | (N = 159) | (N = 386) |
| Preferred Term (PT) | n (%) | n (%) | n (%) | n (%) |
| Cardiac disorders | | | | |
| Atrial fibrillation | 2 (1.0) | 1 (0.6) | 0 | 3 (0.8) |
| Sinus bradycardia | 1 (0.5) | 4 (2.6) | 2 (1.3) | 7 (1.8) |
| Ventricular extrasystoles | 0 | 0 | 2 (1.3) | 2 (0.5) |
| Bundle branch block left | 0 | 1 (0.6) | 1 (0.6) | 2 (0.5) |
| Bradycardia | 0 | 0 | 1 (0.6) | 1 (0.3) |
| Sinus arrhythmia | 0 | 0 | 1 (0.6) | 1 (0.3) |
| Atrioventricular block first degree | 1 (0.5) | 0 | 0 | 1 (0.3) |
| Tachycardia | 1 (0.5) | 0 | 0 | 1 (0.3) |
| Investigations | | | | |
| Electrocardiogram QT prolonged | 0 | 3 (1.9) | 3 (1.9) | 6 (1.6) |
| Electrocardiogram ST segment depression | 0 | 2 (1.3) | 0 | 2 (0.5) |
| Electrocardiogram abnormal | 0 | 1 (0.6) | 0 | 1 (0.3) |

MedDRA = Medical Dictionary for Regulatory Activities
Note:
Adverse events are coded using MedDRA version 18.1.
If a patient had more than one event that coded to the same MedDRA category, the patient was counted only once in the MedDRA category.
For patients who were randomized to placebo then rerandomized to AVP-786, adverse events were counted based on the treatment they were on when the event occurred.

### 6.5.2. Vital Signs

In the 12-week Parallel Groups (placebo, AVP-786-18, or AVP-786-28), there were no notable mean or median change from Baseline to any postbaseline visit in the standing or supine position for systolic blood pressure, diastolic blood pressure, heart rate, respiration rate, or temperature.

Orthostatic blood pressure measurements were required with Protocol Amendment 3. In the 12-week Parallel Groups, there were no notable mean or median changes from supine to standing at any postbaseline visit. Heart rate increases upon standing were up to 6% but similar in all treatment groups.

In general, the proportions of patients experiencing PCS vital signs abnormalities were similar in the All AVP-786 and All Placebo treatment groups (Table 54).

The proportion of patients with PCS orthostatic hypotension was high in all groups (16.8%, 19.1%, and 19.4% in the All Placebo, All AVP-786-18, and All AVP-786-28 groups, respectively; Table 55).

Overall, 15 patients (3.9%) had TEAEs of dizziness; by treatment group, the proportions of patients with TEAEs of dizziness were similar (2.6%, 3.2%, and 3.1% in the All Placebo, All AVP-786-18, and All AVP-786-28 groups, respectively). Four patients (1.0%) had TEAEs of syncope, 2 (0.5%) had TEAEs of hypotension, 2 (0.5%) had TEAEs of orthostatic hypotension, and 1 (0.3%) had a TEAE of presyncope; the proportions of patients with these TEAEs were similar across treatment groups.

TABLE 54
Potentially Clinically Significant Postbaseline Vital Sign Abnormalities (Safety Population, Part 2)

| | | All Placebo | | All AVP-786-18 | | All AVP-786-28 | | All Patients | | |
|---|---|---|---|---|---|---|---|---|---|---|
| | | (N = 194) | | (N = 156) | | (N = 159) | | (N = 386) | | |
| Parameter | PCS Criterion | N1 | n (%) | N1 | n (%) | N1 | n (%) | N1 | n (%) | |
| Systolic Blood | ≤90 and ≥20 decrease | 191 | 4 (2.1) | 154 | 3 (1.9) | 159 | 4 (2.5) | 382 | 11 | (2.9) |
| Pressure (SBP) | from Baseline | | | | | | | | | |
| | >180 and ≥20 increase | 191 | 2 (1.0) | 154 | 1 (0.6) | 159 | 0 | 382 | 3 | (0.8) |
| | from Baseline | | | | | | | | | |
| Diastolic Blood | ≤50 and ≥15 decrease | 191 | 5 (2.6) | 154 | 2 (1.3) | 159 | 0 | 382 | 7 | (1.8) |
| Pressure (DBP) | from Baseline | | | | | | | | | |
| | ≥105 and ≥15 increase | 191 | 0 | 154 | 1 (0.6) | 159 | 0 | 382 | 1 | (0.3) |
| | from Baseline | | | | | | | | | |
| Heart Rate (HR) | ≤50 and ≥15 decrease | 191 | 2 (1.0) | 154 | 2 (1.3) | 159 | 3 (1.9) | 382 | 7 | (1.8) |
| | front Baseline | | | | | | | | | |
| | ≥120 and ≥15 increase | 191 | 0 | 154 | 0 | 159 | 0 | 382 | 0 | |
| | front Baseline | | | | | | | | | |
| SBP and HR | SBP ≥10 and HR ≥5 | 191 | 72 (37.7) | 154 | 58 (37.7) | 159 | 73 (45.9) | 382 | 188 | (49.2) |
| | increase from Baseline | | | | | | | | | |
| DBP and HR | DBP ≥5 and HR ≥5 | 191 | 110 (57.6) | 154 | 86 (55.8) | 159 | 86 (54.1) | 382 | 248 | (64.9) |
| | increase from Baseline | | | | | | | | | |

Note:
For patients who were randomized to placebo then rerandomized to AVP-786, patients will be counted based on the treatment they were on when the PCS criterion was met. For patients who were randomized to placebo then rerandomized to AVP-786, Baseline for the placebo portion is the last assessment prior to first dose and Baseline for the AVP-786 portion is the last assessment prior to rerandomization.
Criteria involving multiple parameters are included only if they take place at the same visit.
1The number of patients who had vital signs performed per group.

TABLE 55
Potentially Clinically Significant Orthostatic Hypotension and Postural Tachycardia (Safety Population, Part 2)

| | All Placebo | | All AVP-786-18 | | All AVP-786-28 | | All Patients | |
|---|---|---|---|---|---|---|---|---|
| | (N = 194) | | (N = 156) | | (N = 159) | | (N = 386) | |
| PCS Criterion | N1 | n (%) | N1 | n (%) | N1 | n (%) | N1 | n (%) |
| Orthostatic hypotension - decrease of ≥20 mmHg | 155 | 26 (16.8) | 131 | 25 (19.1) | 134 | 26 (19.4) | 323 | 72 (22.3) |
| in SBP or ≥10 mmHg in DBP change from supine to standing | | | | | | | | |
| Postural tachycardia - increase of ≥30 bpm in HR | 155 | 1 (0.6) | 131 | 3 (2.3) | 134 | 0 | 323 | 4 (1.2) |
| change from supine to standing or standing HR ≥120 bpm | | | | | | | | |

Note:
For patients who were randomized to placebo then rerandomized to AVP-786, patients will be counted based on the treatment they were on when the PCS criterion was met. For patients who were randomized to placebo then rerandomized to AVP-786, Baseline for the placebo portion is the last assessment prior to first dose and Baseline for the AVP-786 portion is the last assessment prior to rerandomization.
Criteria involving multiple parameters will be included only if they take place at the same visit.
1The number of patients who had orthostatic vital signs performed per group.

### 6.5.3. Sheehan Suicidality Tracking Scale

There was no evidence of an increase in suicidal behavior or ideation in any treatment group based on postbaseline assessments of the S-STS.

### 6.5.4. Mini Mental State Examinations

There was no evidence of clinically significant mean or median change in cognition in any treatment group, as measured by the MMSE Total scores. The mean (SD) changes from Baseline to Week 12 in the 12-week Parallel Groups were 0.1 (2.8), 0.5 (3.2), and 0.1 (3.0) for the placebo, AVP-786-18, and AVP-786-28 groups, respectively. There were no TEAEs related to worsening cognition.

### 6.5.5. Timed Up and Go Test

There was no evidence of clinically significant mean or median changes in the TUG test in any group. In all 12-week Parallel Groups, the mean change from Baseline at Week 12 was <1 second.

### 6.5.6. Epworth Sleepiness Scale

There was no evidence of clinically significant mean or median change in sleepiness in any treatment group, as measured by the ESS Total scores. The median (minimum, maximum) percent changes from Baseline to Week 12 in the 12-week Parallel Groups were −1.0 (−8, 12), −1.0 (−11, 10), and 0.0 (−13, 10) for the placebo, AVP-786-18, and AVP-786-28 groups, respectively.

TEAEs for the All Placebo, All AVP-786-18, and All AVP-786-28 groups related to sleepiness were somnolence (3.6%, 3.8%, and 2.5%, respectively), fatigue (1.5%, 0.6%, and 1.3%, respectively), and lethargy (0%4, 0.6%, and 0.6%, respectively).

### 6.6. Safety Conclusions

Patients in the All AVP-786-18 group had a higher incidence of TEAEs compared with the All Placebo group (51.3% vs 43.8%, respectively); however, the incidence of TEAEs was similar between the All AVP-786-28 and All Placebo groups (45.3% and 43.8%, respectively). The most frequently experienced TEAEs (≥5% of patients in any treatment group) that occurred in a higher percentage of patients in the All AVP-786-18 group were:
Fall (6.2%, 9.6%, and 7.5% for the All Placebo, All AVP-786-18, and All AVP-786-28 groups, respectively)
Urinary tract infection (5.7%, 7.1%, and 3.1%, respectively)
Diarrhoea (2.6%, 6.4%, and 4.4%, respectively)
Headache (1.5%, 6.4%, and 1.3%, respectively).

Agitation was experienced in a higher percentage of patients in the All Placebo and All AVP-786-28 groups compared with the All AVP-786-18 group (5.2%, 5.0%, and 2.6%, respectively).

Patients in the All AVP-786-18 and All AVP-786-28 groups had a higher incidence of drug-related TEAEs compared with the All Placebo group (17.3%, 14.5%, and 10.8%, respectively). The most frequently experienced drug-related TEAEs (≥2% of patients in any treatment group) were diarrhoea (1.5%, 2.6%, and 1.9% in the All Placebo, All AVP-786-18, and All AVP-786-28 groups, respectively) and somnolence (1.5%, 3.2%, and 2.5%, respectively).

A higher percentage of patients in the All AVP-786-18 group discontinued study treatment due to TEAEs compared with the All Placebo group (5.8% and 3.1%, respectively); however, the percentage of patients was similar between the All AVP-786-28 and All Placebo groups (3.1% and 3.1%, respectively). The most frequently experienced TEAEs (≥2 patients in any treatment group) leading to discontinuation of study treatment that occurred in a higher number of patients in the All AVP-786 groups were:
Fall (0, 2 [1.3%], and 2 [1.3%] for All Placebo, All AVP-786-18, and All AVP-786-28 groups, respectively)
Electrocardiogram QT prolonged (0, 1 [0.6%], and 2 [1.3%], for All Placebo, All AVP-786-18, and All AVP-786-28 groups, respectively).

Patients in the All AVP-786-18 group had a higher incidence of SAEs compared with the All Placebo group (10.9% and 3.1%, respectively); however, the incidence of SAEs was similar between the All AVP-786-28 and All Placebo groups (4.4% and 3.1%, respectively). A total of 2 serious drug-related SAEs, bradycardia and fall, were experienced in 2 patients in the All AVP-786-28 group.

Five patients (1.3%) died during the study, including 1 (0.5%), 3 (1.9%), and 1 (0.6%) in the All Placebo, All AVP-786-18, and All AVP-786-28 groups, respectively. None of the deaths were considered related to study drug by the Investigator.

The TEAEs of interest for AVP-786 were events of fall, sinus bradycardia, rash, thrombocytopenia, and serotonin syndrome.
Fall was the most frequently experienced TEAE across all treatment groups; 10.1% patients experienced TEAEs of fall (6.2%, 9.6%, and 7.5% for the All Placebo, All AVP-786-18, and All AVP-786-28 groups, respectively). TEAEs of fall were generally mild to moderate in severity. Few falls were experienced as SAEs (0.5%), led to discontinuation of study drug (1.0%), or were considered related to study drug (1.3%).
For the remaining TEAEs of interest, low number of patients experienced these types of events. Eight patients experienced sinus bradycardia events (0.5%, 2.6%, and 1.9% for the All Placebo, All AVP-786-18, and All AVP-786-28 groups, respectively). Four patients experienced rash events (0%, 1.3%, and 1.3%, respectively). One patient treated with AVP-786-18 experienced a TEAE of thrombocytopenia. No patients experienced TEAEs of serotonin syndrome.

No additional safety risks were identified by clinical laboratory tests, ECGs, or vital signs. No increased risk of cognitive decline, somnolence/sedation, or suicidality was observed in patients treated with AVP-786 compared with patients treated with placebo.

### 7. Discussion and Overall Conclusions

This study was a Phase 3, multicenter, randomized, double-blind, placebo-controlled study to assess the efficacy, safety, and tolerability of AVP-786 for the treatment of patients with clinically significant, moderate/severe agitation associated with dementia of the Alzheimer's type.

The efficacy, safety, and tolerability of 2 dose levels of AVP-786 (AVP-786-18 and AVP-786-28) were compared to placebo by using a 2-stage SPCD design that minimized the effect of placebo response on the statistical outcome. The SPCD rerandomization was completed in a double-blinded fashion (i.e., neither Investigators nor patients were aware of the SPCD rerandomization). The randomized, placebo-controlled, double-blind, SPCD is designed to reduce sources of bias in neurobehavioral clinical studies, which have a high expected placebo response. Potentially high responses observed among placebo-treated patients can constitute a significant challenge for drug development in studies of behavioral and psychiatric disorders. The SPCD is essentially comprised of 2 randomized trials (stages) run one after another; Stage 1 includes all patients randomized and Stage 2 rerandomizes those who were Placebo Nonresponders during Stage 1 to active drug or placebo. The expectation is that signal detection will be enhanced by including only data from Placebo Nonresponders in the primary analysis, which is comprised of pooled data from Stage 1 and Stage 2.

For Stage 1, 387 patients were randomized to treatment in a 2:1:1 (placebo:AVP-786-18: AVP-786-28) ratio, and 382 of them were included in the mITT population for Stage 1, with 191, 94, and 97 mITT patients in the placebo, AVP-786-18, and AVP-786-28 groups, respectively. In Stage 2, 125 Placebo Nonresponders were rerandomized to treatment (1:1:1) and qualified for the mITT population. Most patients completed the study.

The groups in the mITT population were well balanced with regard to sex, race, ethnicity, and age. A higher proportion of patients in the AVP-786-18 group (16.0%) were <65 years of age than in the placebo (7.3%) or AVP-786-28 (9.3%) groups.

Although neither dose of AVP-786 showed a statistically significant difference from placebo in the CMAI Total score or mADCS-CGIC-Agitation score based on the FWE α=0.05 level, these comparisons were significant for the AVP-786-18 dose at the nominal α=0.05 level (p=0.008 and p=0.012, respectively). Based on the overall SPCD analyses, patients treated with AVP-786-18 showed significant improvement from Baseline in measures of agitation compared to patients in the placebo group (nominal significance at p<0.05):
CMAI Total score (primary efficacy endpoint)
Three CMAI subscales scores—F1—Aggressive Behavior, F2—Physically Nonaggressive Behavior, and F3—Verbally Agitated Behavior
mADCS-CGIC-Agitation score (the key secondary efficacy endpoint)
NPI—Irritability/Lability Domain score
CGIS-Agitation score
PGIC score
DEMQOL-Proxy Total score

Sensitivity analyses of the CMAI endpoints supported the primary findings. The 12-week Parallel Group analyses consistently showed mean treatment differences similar to those observed in the SPCD analysis, but they were not consistently significant.

### Pharmacokinetics

There did not appear to be any significant changes in d6-DM, d3-DX, d3-3-MM, and Q plasma concentrations from Week 6 to Week 12. Exposures to d6-DM and metabolites increased with the increase in d6-DM dose in AVP-786.

### Safety

Treatment with AVP-786-18 and AVP-786-28 was generally well tolerated during the study. Patients in the All AVP-786-18 group had a higher incidence of TEAEs compared with the All Placebo group; however, the incidence of TEAEs was similar between the All AVP-786-28 and All Placebo groups. The most frequently experienced TEAEs that occurred in a higher percentage of patients in the All AVP-786-18 group were fall, urinary tract infection, diarrhoea, and headache; however, few were considered related to study drug or led to treatment discontinuation

Overall, the incidence of discontinuations due to TEAEs (5.2%) and the incidence SAEs (7.8%) and was low for a 12-week study in an elderly patient population. A higher percentage of patients in the All AVP-786-18 group discontinued study treatment due to TEAEs or experienced SAEs compared with the All Placebo group; however, the percentages of patients were similar between the All AVP-786-28 and All Placebo groups. Fall and electrocardiogram QT prolonged (each experienced for 2 [1.3%] patients) were the only TEAEs that led to discontinuation of more than 1 patient in the AVP-786 treatment groups.

The most frequently experienced SAEs that occurred in a higher number of patients in the All AVP-786-18 group were urinary tract infection, alcohol poisoning, and cerebrovascular accident; however, no SAE was experienced by more than 2 patients in a single group.

Five patients (1.3%) died during the study, including 1, 3, and 1 patient in the All Placebo, All AVP-786-18, and All AVP-786-28 groups, respectively. None of the deaths were considered related to study drug by the Investigator.

The TEAEs of interest for AVP-786 were events of fall, sinus bradycardia, rash, thrombocytopenia, and serotonin syndrome. Fall was the most frequently experienced TEAE across all treatment groups. Thirty-nine (10.1%) patients experienced TEAEs of fall (6.2%, 9.6%, and 7.5% for the All Placebo, All AVP-786-18, and All AVP-786-28 groups, respectively). These TEAEs were generally mild to moderate in severity, and few were experienced as SAEs, led to discontinuation of study drug, or were considered related to study drug. Other TEAEs of interest were uncommon, were rarely severe or serious, and were rarely the cause of discontinuation.

No additional safety risks were identified by clinical laboratory tests, ECGs, or vital signs. No increased risk of cognitive decline, somnolence/sedation, or suicidality was observed in patients treated with AVP-786 compared with patients treated with placebo.

Overall Conclusions:
Treatment with AVP-786-18 demonstrated significant improvement (at the nominal level) in several measures of agitation compared with placebo in patients with Alzheimer's dementia, including the primary efficacy endpoint CMAI Total score (p=0.008) and the key secondary efficacy endpoint mADCS-CGIC-Agitation score (p=0.012). Treatment with AVP-786-28 showed some numeric improvement in several measures of agitation. The totality of the data from this study provides evidence that AVP-786 is beneficial in reducing agitation in patients with Alzheimer's dementia.
AVP-786 was safe and generally well tolerated at both dose levels. The incidences of TEAEs, drug-related TEAEs, SAEs, and discontinuations due to TEAEs were similar in the All Placebo and All AVP-786-28 groups, but higher in the All AVP-786-18 group. No additional safety risks were identified by clinical laboratory tests, ECGs, or vital signs. No increased risk of cognitive decline, somnolence/sedation, or suicidality was observed in patients treated with AVP-786 compared with patients treated with placebo.

### Example 4

A Phase 3, multicenter, randomized, double-blind, placebo-controlled study to assess the efficacy, safety, and tolerability of AVP-786 (deuterated [d6]-dextromethorphan hydrobromide [d6-dm]/quinidine sulfate [Q]) for the treatment of agitation in patients with dementia of the Alzheimer's type.

### List of Abbreviations and Definitions of Terms

The following abbreviations and specialized terms are used in this Example 4.

TABLE 56
Abbreviations and Specialized Terms

| | |
|---|---|
| AA | Alzheimer's Association |
| AD | Alzheimer's disease |
| ADAS-cog | Alzheimer's Disease Assessment Scale-cognitive subscale |
| ADCS-CGIC-Overall | Alzheimer's Disease Cooperative Study-Clinical Global Impression of Change |
| | for Overall Clinical Status |
| AE | adverse event |
| ALT | alanine aminotransferase |
| ANCOVA | analysis of covariance |
| AR1 | first-order autoregressive |
| AST | aspartate aminotransferase |
| AUC | area under the concentration-time curve |
| BP | blood pressure |
| BUN | blood urea nitrogen |
| CFR | US Code of Federal Regulations |
| CGI | Clinical Global Impressions |
| CGIC-Overall | Alzheimer's Disease Cooperative Study-Clinical Global Impression of Change |
| | for Overall Clinical Status |
| CGIS-Agitation | Clinical Global Impression of Severity of Illness scale for Agitation |
| CI | confidence interval |
| CK | creatine kinase |
| CMAI | Cohen-Mansfield Agitation Inventory |
| Cₘₐₓ | maximum plasma concentration |
| CNS | central nervous system |
| CS | compound symmetry |
| CSDD | Cornell Scale for Depression in Dementia |
| CYP | cytochrome P450 |
| d3-3-MM | d3-3-methoxy morphinan |
| d3-DX | d3-dextrorphan |
| d6-DM | deudextromethorphan hydrobromide (or deudextromethorphan) |
| DEMQOL | Dementia Quality of Life |
| DSM-IV-TR | Diagnostic and Statistical Manual of Mental Disorders, 4th Edition, Text |
| | Revision |
| DSMB | Data Safety Monitoring Board |
| ECDEU | Early Clinical Drug Evaluation Unit |
| ECG | electrocardiogram |
| eCRF | electronic case report form |
| EQ-5D-5L | EuroQol 5-Dimension 5-Level |
| ESS | Epworth Sleepiness Scale |
| ET | early termination |
| FDA | Food and Drug Administration (US) |
| FWE | family-wise error |
| GCP | Good Clinical Practice |
| GEE | general estimating equation |
| GGT | gamma-glutamyl transferase |
| GMHR | General Medical Health Rating |
| GMP | Good Manufacturing Practice |
| HbA1c | glycosylated hemoglobin |
| HR | heart rate |
| ICF | informed consent form |
| ICH | International Council for Harmonisation |
| IEC | Independent Ethics Committee |
| IPA | International Psychogeriatric Association |
| IRB | Institutional Review Board |
| ITT | intent-to-treat |
| IWRS | interactive web-response system |
| LAR | legally authorized representative |
| LDH | lactate dehydrogenase |
| LOCF | last observation carried forward |
| LS | least-square |
| mADCS-CGIC-Agitation | modified Alzheimer's Disease Cooperative Study-Clinical Global Impression of |
| | Change scale for Agitation |
| MAOI | monoamine oxidase inhibitor |
| MAR | missing at random |
| MedDRA | Medical Dictionary for Regulatory Activities |
| MI | multiple imputation |
| mITT | modified intent-to-treat |
| MMRM | mixed model repeated measures |
| MMSE | Mini Menial State Examination |
| MNAR | missing not at random |
| NIA | National Institute on Aging |
| NINCDS-ADRDA | National Institute of Neurological and Communicative Disorders and Stroke and |
| | Alzheimer's Disease and Related Disorders Association |
| NMDA | N-methyl-D-aspartate |
| NPI | Neuropsychiatric Inventory |
| NPI-NH | NPI nursing home version |
| PCS | potentially clinically significant |
| PGIC | Patient Global Impression of Change |
| PI | Principal Investigator |
| PK | pharmacokinetic |
| PMM | pattern mixture models |
| PT | preferred term |
| PVC | premature ventricular contraction |
| Q | quinidine sulfate (or quinidine) |
| QOL | quality of life |
| QTc | corrected QT |
| QTcF | QT corrected using Fridericia's method |
| RBC | red blood cell |
| RUD | Resource Utilization in Dementia |
| SAE | serious adverse event |
| SAP | statistical analysis plan |
| SD | standard deviation |
| SES | standard effect size |
| SOC | System Organ Class |
| SNRI | serotonin-norepinephrine reuptake inhibitor |
| SSRI | selective serotonin reuptake inhibitors |
| S-STS | Sheehan Suicidality Tracking Scale |
| TEAE | treatment-emergent adverse events |
| T3 | triiodothyronine |
| T4 | thyroxine |
| TSH | thyroid-stimulating hormone |
| TUG | Timed Up and Go |
| US | United States |
| WBC | white blood cell |
| WOCF | worst observation carried forward |
| ZBI | Zarit Burden Interview |

### 1. Introduction

### 1.1. AVP-786

AVP-786 is a combination product of deudextromethorphan hydrobromide (d6-DM), a central nervous system (CNS) active agent, and quinidine sulfate (Q), used as an inhibitor of d6-DM metabolism via the cytochrome P450 (CYP) liver isoenzyme 2D6 (CYP2D6).

### 2. Investigational Plan

### 2.1. Overall Study Design and Plan: Description

This was a Phase 3, multicenter, randomized, double-blind, placebo-controlled study of 12-week treatment duration. Approximately 470 patients (190 randomized to placebo and 280 randomized to AVP-786-28 or AVP-786-42.63) were to be enrolled at approximately 75 centers in North America. There were 8 scheduled clinic visits including a Screening Visit, and 2 safety follow-up phone calls in this study. Patients attended clinic visits at Screening, Baseline (Day 1), and on Days 8 (Visit 2/Week 1), 15 (Visit 2.1/Week 2), 22 (Visit 3/Week 3), 43 (Visit 4/Week 6), 64 (Visit 5/Week 9), and 85 (Visit 6/Week 12). Patients who terminated early received daily phone calls for 5 consecutive days following the early termination (ET) visit to query on their overall well-being and were asked to return for an in-clinic Follow-up visit 30 days after last dose of study drug for selected safety and efficacy assessments. Patients who did not roll over to the extension study (Study 15-AVP-786-303) received a safety follow-up phone call 30 days after the last dose of study drug. Safety follow-up phone calls were also made on Days 29 (Week 4) and 71 (Week 10). Study procedures were performed at each visit as outlined in the Study Schedule (Table 59).

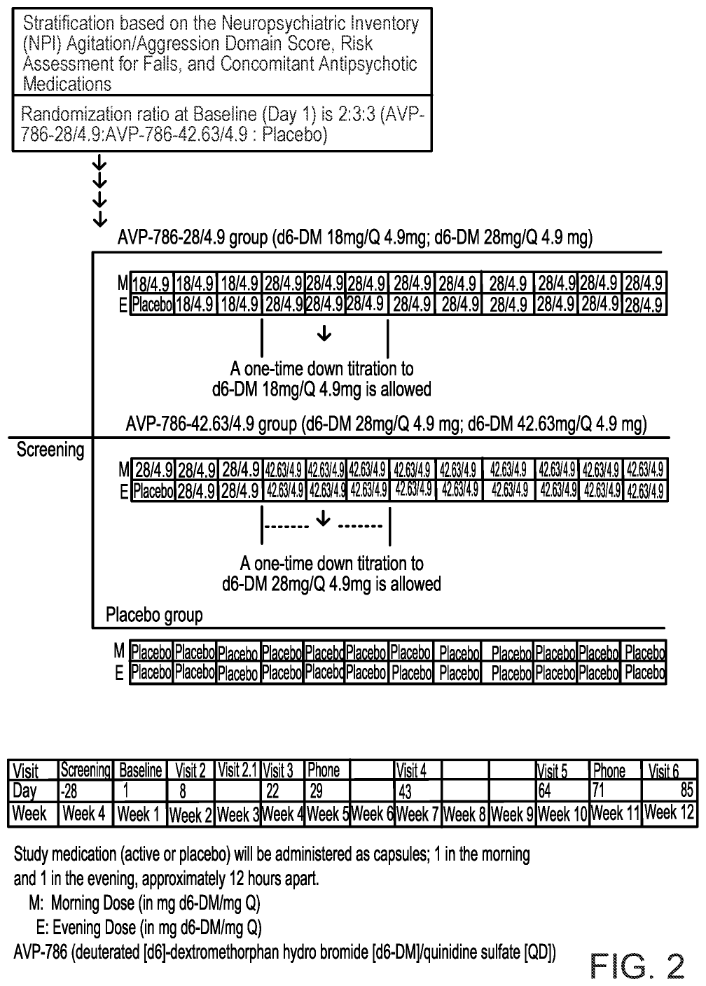

Eligible patients were randomly assigned at the Baseline visit to receive AVP-786 or matching placebo (FIG. 2). Study drug was administered orally twice daily from Baseline (Day 1) through Week 12 (Day 85). Patients (or caregivers) self-administered study drug on all study days except on applicable clinic visit days, when patients were administered their morning dose of study drug at the clinic in the presence of site personnel, regardless of the time of day. Screening occurred within 4 weeks prior to randomization.

Following Screening procedures for assessment of inclusion and exclusion criteria, eligible patients were randomized to receive either AVP-786-28 (d6-DM 28 mg/Q 4.9 mg), AVP-786-42.63 (d6-DM 42.63 mg/Q 4.9 mg), or placebo. (Prior to Protocol Amendment 4, patients were randomized to placebo or AVP-786-28 in a 1:1 ratio; with Amendment 4, randomization was changed to placebo, AVP-786-28, or AVP-786-42.63 in an approximately 3:2:2 ratio.) The randomization was stratified by the Neuropsychiatric Inventory (NPI)-Agitation/Aggression domain score (≤6 vs >6), risk assessment for falls (normal/mild vs moderate/severe), and concomitant use of antipsychotic medications (yes vs no). Study drug (active or placebo) was administered orally twice daily (1 capsule in the morning and 1 capsule in the evening approximately 12 hours apart) throughout the treatment period.

Patients randomized to the AVP-786-28 group started with AVP-786-18 once a day in the morning and placebo in the evening for the first 7 days of the study. From Day 8, patients received AVP-786-18 twice daily for 14 days. From Day 22, patients received AVP-786-28 twice daily for the remaining 9 weeks of the study. If deemed necessary by the Investigator, a one-time downward dose adjustment to AVP-786-18 was allowed after Visit 3 up to and including Visit 4 (i.e., Day 23 to Day 43), and the patient had to remain on the lower dose of study drug for the remainder of the study. Patients who required a dose adjustment between study visits needed to have an unscheduled visit to perform safety assessments.

Patients randomized to the AVP-786-42.63 group started with AVP-786-28 once a day in the morning and placebo in the evening for the first 7 days of the study. From Day 8, patients received AVP-786-28 twice daily for 14 days. From Day 22, patients received AVP-786-42.63 twice daily for the remaining 9 weeks of the study. If deemed necessary by the Investigator, a one-time downward dose adjustment to AVP-786-28 was allowed after Visit 3 up to and including Visit 4 (i.e., Day 23 to Day 43), and the patient had to remain on the lower dose of study drug for the remaining study duration. Patients requiring a dose adjustment between study visits were required to have an unscheduled visit to perform safety assessments.

### 2.2. Discussion of Study Design, Including the Choice of Control Groups

The randomized, placebo-controlled, double-blind design was selected to reduce sources of bias that are inherent in less well-controlled designs. The safety assessments used are standard in clinical research and are generally recognized as reliable, accurate, and relevant. The rating scales used to assess efficacy are well-established instruments that are widely used in clinical studies of Alzheimer's disease.

### 2.3. Selection of Study Population

### 2.3.1. Inclusion Criteria

For inclusion into the trial, patients were required to fulfill all of the following criteria:
1. Males and females 50 to 90 years of age, inclusive, at the time of informed consent.
2. Diagnosis of probable Alzheimer's disease according to the 2011 National Institute on Aging-Alzheimer's Association (NIA-AA) working groups criteria. Either outpatients or residents of an assisted-living facility or a skilled nursing home.
3. The patient had to have clinically significant, moderate/severe agitation at the time of Screening and for at least 2 weeks prior to randomization, that interfered with daily routine and for which a prescription medication was indicated, in the opinion of the Investigator.
4. The diagnosis of agitation must have met the International Psychogeriatric Association (IPA) provisional definition of agitation.
5. Clinical Global Impression of Severity of Illness scale for Agitation (CGIS-Agitation) score ≥4 (moderately ill) at Screening and Baseline.
6. Mini Mental State Examination (MMSE) score between 6 and 26 (inclusive) at Screening and Baseline.
7. The patient had to have stable cardiac, pulmonary, hepatic, and renal function.
8. The patient had to have an electrocardiogram (ECG; obtained within the past month prior to randomization and evaluated by a central ECG reader) with no clinically significant findings.
9. If female of childbearing potential, must have been practicing a medically-acceptable method of birth control for at least 1 month prior to randomization and continued with the same method during the entire study duration (oral contraceptive tablets, hormonal implant device, hormone patch, intrauterine device, diaphragm and contraceptive cream or foam, condom with spermicide, or abstinence) or to have been surgically sterile or postmenopausal.
10. Use of medication for the treatment of Alzheimer's disease (e.g., donepezil, rivastigmine, galantamine, memantine) was allowed provided the dose had been stable for at least 3 months prior to randomization.
11. Concomitant use of antidepressants such as selective serotonin reuptake inhibitors (SSRIs; e.g., fluoxetine, sertraline, citalopram), serotonin-norepinephrine reuptake inhibitors (SNRIs; e.g., venlafaxine, desvenlafaxine, duloxetine) was allowed, provided the dose had been stable for at least 1 month prior to randomization and was within the Package Insert guidance for that medication. Paroxetine, a CYP2D6 substrate, was allowed provided the dose did not exceed 10 mg/day.
12. Concomitant use of hypnotics at bedtime (e.g., eszopiclone, zolpidem, zaleplon, trazodone [up to 50 mg/day]) for the nighttime treatment of insomnia was allowed, provided the dose had been stable for at least 1 month prior to randomization and remained stable throughout the study. In addition, concomitant use of short acting benzodiazepines (e.g., midazolam, oxazepam, low dose alprazolam [up to 0.5 mg/day]) for behavioral disturbances was allowed.
13. Patients who were concurrently taking allowed medications for the treatment of agitation secondary to Alzheimer's disease (e.g., atypical antipsychotics, antidepressants, buspirone) were eligible provided they had been on a stable dose for at least 2 weeks prior to Screening and at least 1 month prior to randomization.
14. Patient must not have shown current and significant symptoms of a depressive disorder and had to have a score <10 in the Cornell Scale for Depression in Dementia (CSDD) at Screening.
15. Patient had to have no history or current clinical symptoms of schizophrenia, schizoaffective disorder, or bipolar disorder, as defined in the Diagnostic and Statistical Manual of Mental Disorders, 4th Edition, Text Revision (DSM-IV-TR).
16. Caregiver had to be willing and able to comply with study procedures, including not administering any prohibited medications during the course of the study.
17. Patient/caregiver must have been willing to sign and receive a copy of patient/caregiver ICF after the nature and risks of study participation had been fully explained. Patients who were not capable of signing the ICF but were able to provide assent, or the patient's authorized representative agreed to participation (for patients unable to provide assent) were allowed.

### 2.3.2. Exclusion Criteria

Any of the following was regarded as a criterion for exclusion from the trial:
1. Caregiver was unwilling or unable, in the opinion of the Investigator, to comply with study instructions.
2. Patient had dementia predominantly of non-Alzheimer's type (e.g., vascular dementia, frontotemporal dementia, Parkinson's disease, substance-induced dementia).
3. Patients with symptoms of agitation that were not secondary to Alzheimer's disease (e.g., secondary to pain, other psychiatric disorder, or delirium).
4. Patients with myasthenia gravis (contraindication for quinidine).
5. Patients with any personal history of complete heart block, QTc prolongation, or torsades de pointes.
   Screening and Baseline QT corrected using Fridericia's method (QTcF) of >450 msec for males and >470 msec for females based on central review unless due to ventricular pacing Presence of premature ventricular contractions (PVCs) as evaluated by a central reader and deemed clinically significant by the Investigator
6. Patients with any family history of congenital QT interval prolongation syndrome.
7. Patients with known hypersensitivity to DM, Q, opiate drugs (codeine, etc.), or any other ingredient of the study drug.
8. Patients with history of allergy to benzodiazepines (e.g., lorazepam).
9. Patients who had ever received DM co-administered with Q.
10. Patients who had been taking disallowed concomitant medications within 2 weeks or 5 half-lives, whichever was longer, prior to Baseline.
11. Patients with co-existent clinically significant or unstable systemic diseases that could confound the interpretation of the safety results of the study (e.g., malignancy [except skin basal-cell carcinoma or untreated prostate cancer], poorly controlled diabetes, poorly controlled hypertension, unstable pulmonary, renal or hepatic disease, unstable ischemic cardiac disease, dilated cardiomyopathy, or unstable valvular heart disease). Certain other non-metastatic cancer could be allowed. Each case had to be evaluated individually with the Medical Monitor.
12. Patients who were currently participating in, or who had participated in other interventional (drug or device) clinical study within 30 days of Baseline.
13. Patients with history of postural syncope or any history of unexplained syncope (evaluated on a case by case basis) within 12 months of Baseline.
14. Patients with a history of substance and/or alcohol abuse within the past 1 year.
15. Patients determined to have a high imminent risk of falls during the study based on a clinical evaluation by the Investigator.
16. Patients with evidence of serious risk of suicide at Screening and Baseline based on the Sheehan Suicidality Tracking Scale (S-STS), i.e., a score of 3 or 4 on any one question 2 through 6 or 11 or a score of 2 or higher on any one questions 1a, 7 through 10, or 12, or who, in the opinion of the Investigator, presented a serious risk of suicide.

### 2.3.3. Removal of Patients from Therapy or Assessment

Patients and caregivers were advised verbally and in the written ICF that they had the right to withdraw from the study at any time without prejudice or loss of benefits to which they were otherwise entitled. The Investigator or Sponsor could discontinue a patient from the study in the event of an intercurrent illness, adverse event, other reasons concerning the health or well-being of the patient, or in the case of lack of cooperation, noncompliance, protocol violation, or other administrative reasons. If a patient did not return for a scheduled visit, every effort was to be made to contact the patient. Regardless of the circumstance, every effort was to be made to document patient outcome, if possible. The Investigator was to inquire about the reason for withdrawal, request the caregiver return all unused study drug, and follow-up with the patient regarding any unresolved adverse events.

In addition, patients who presented a QTcF>500 msec (unless due to ventricular pacing) or a QTcF interval change from the predose Baseline ECG of >60 msec at any time after randomization were withdrawn from the study. The QTcF values were assessed for clinical significance and recorded.

Patients who terminated early were to be asked to return to the clinic to complete the Visit 6 assessments and an in-clinic Follow-up visit, 30 days after last dose of study drug for selected safety and efficacy assessments. In addition, daily phone calls for 5 consecutive days following ET visit were to be made for these patients to assess their overall well-being.

If the patient withdrew from the study and consent was withdrawn by the caregiver and/or patient's representative for disclosure of future information, no further evaluations were performed, and no additional data were collected. The Sponsor could retain and continue to use any data that had been collected before such withdrawal of consent. Patients who withdrew from the study were not replaced.

### 2.4. Treatments

### 2.4.1. Treatments Administered

Clinical study drug was provided as hard, printed, opaque, blue, gelatin capsules (size 3). Each capsule of the study drug contained 1 of the following:
AVP-786-42.63, 42.63 mg of d6-DM and 4.9 mg of Q (USP, EP) (referred to as AVP-786-42.63)
AVP-786-28, 28 mg of d6-DM and 4.9 mg of Q (USP, EP) (referred to as AVP-786-28)
AVP-786-18, 18 mg of d6-DM and 4.9 mg of Q (USP, EP) (referred to as AVP-786-18)
AVP-786 matching placebo, with the same excipients as the study drug (referred to as placebo)

Drug supplies were provided to the site in double-blind, individual, prelabeled blister cards.

### 2.4.2. Identity of Investigational Product(s)

AVP-786 was supplied as 42.63 mg of d6-DM and 4.9 mg of Q (AVP-786-42.63), 28 mg of d6-DM and 4.9 mg of Q (AVP-786-28), or 18 mg of d6-DM and 4.9 mg of Q (AVP-786-18) in hard, printed, opaque, blue, gelatin capsules for oral administration. The qualitative and quantitative compositions of the 2 doses of AVP-786 and placebo are listed in Table 57.

TABLE 57
Composition of Investigational Product

| Ingredient (amounts in mg) | AVP-786-42.63 | AVP-786-28 | AVP-786-18 | Placebo |
|---|---|---|---|---|
| d6-Dextromethorphan hydrobromide | 42.63 | 28.00 | 18.00 | 0 |
| Quinidine sulfate USP, EP | 4.90 | 4.90 | 4.90 | 0 |

EP = European Pharmacopoeia;
USP = United States Pharmacopoeia;
NF = National Formulary

### 2.4.3. Method of Assigning Patients to Treatment Groups

Eligible patients were randomized to receive AVP-786-28 capsules, AVP-786-42.63 capsules, or matching placebo capsules on Day 1 (Baseline) in a double-blind manner according to a randomization scheme. The randomization was stratified by NPI—Agitation/Aggression domain score (≤6 vs >6), risk assessment for falls (normal/mild vs moderate/severe), and concomitant use of antipsychotic medications (yes vs no). Blocked randomization was used to ensure treatment balance in each stratum. Patients had at least a 60% chance of receiving AVP-786.

### 2.4.4. Selection of Doses in the Study

The planned doses of AVP-786 for this study initially were d6-DM 28 mg/Q 4.9 mg and d6-DM 18 mg/Q 4.9 mg, hereafter referred to as AVP-786-28 and AVP-786-18, respectively. The AVP-786-28 dose was achieved by gradual titration schedule starting with AVP-786-18. The study protocol was amended to include an additional treatment arm with a dose of d6-DM 42.63 mg/Q 4.9 mg (AVP-786-42.63) achieved by gradual titration with AVP-786-28 using the same titration regimen.

### 2.4.5. Selection and Timing of Dose for Each Patient

All patients received study drug according to the blister card numbers assigned by the IWRS randomization scheme. Designated staff at each site dispensed study drug. Study drug was to be administered to the patient by the caregiver, family member, nursing home staff, or self-administered with supervision, except on applicable clinic visit days when patients were to be administered their dose of study drug at the clinic in the presence of site personnel, regardless of the time of day. Patients and caregivers were instructed that the patient should take the study drug orally with water approximately every 12 hours±4 hours (morning and evening). The time the patient took each dose of medication was to be recorded in the diary card. For Visits 2 (Day 8) and 2.1 (Day 15), caregivers were advised that the patient should take the morning dose of study drug within 2 hours of the clinic appointment. Missed doses were noted in the eCRF. All study drug was supplied and administered in a double-blind manner throughout the entire duration of the study.

Patients beginning active treatment were to be titrated to their randomized dose as follows:
Patients randomized to receive AVP-786-28 were to start with AVP-786-18 once a day in the morning and placebo in the evening for the first 7 days of the study. From Day 8, patients were to receive AVP-786-18 twice daily for 14 days. From Day 22, patients were to receive AVP-786-28 twice daily for the remaining 9 weeks of the study.
Patients randomized to receive AVP-786-42.63 were to start with AVP-786-28 once a day in the morning and placebo in the evening for the first 7 days of the study. From Day 8, patients were to receive AVP-786-28 twice daily for 14 days. From Day 22, patients were to receive AVP-786-42.63 twice daily for the remaining 9 weeks of the study.

### 2.4.6. Blinding

Blinding was to be maintained by providing capsules of the 2 doses of AVP-786 and placebo that were identical in appearance. The Sponsor, patients, caregivers, Investigators, or other study personnel were not to be aware of a patient's treatment assignment.

### 2.4.7. Prior and Concomitant Therapy

Patients were not allowed to take any of the prohibited medications listed in Appendix 1 of the protocol during the study or 2 weeks or 5 half-lives, whichever was longer, before the start of dosing on Day 1. At each visit, caregivers were to be queried as to whether or not the patient had taken any concomitant medications and, if so, the Investigator was to record the medications taken and the reasons for their use. Caregivers were instructed to record concomitant use of rescue medication (lorazepam) in the diary. Concomitant use of P-glycoprotein substrates or of prodrugs whose actions are mediated by the CYP2D6-produced metabolites was to be avoided or, if necessary, carefully monitored.

### 2.4.7.1. Allowed Concomitant Medications

Drugs for the treatment of Alzheimer's disease (e.g., donepezil, rivastigmine, galantamine, memantine) were allowed when administered at stable doses for at least 3 months prior to randomization; the dose of these drugs was to remain unchanged throughout the study. If dose adjustment was necessary, the new dose and the reason for the change were to be recorded.

The use of drugs for the treatment of agitation secondary to Alzheimer's disease (e.g., atypical antipsychotics, antidepressants, buspirone) was allowed, provided the patient had been on a stable dose for at least 2 weeks before Screening and at least 1 month before randomization and throughout the study.

Concomitant use of antidepressants such as SSRIs (e.g., fluoxetine, sertraline, citalopram) or SNRIs (e.g., venlafaxine, desvenlafaxine, duloxetine) was allowed, provided the dose had been stable for at least 1 month prior to randomization and was within the Package Insert guidance for that medication. Paroxetine, a CYP2D6 substrate, was allowed provided the dose did not exceed 10 mg/day. SSRIs, SNRIs, and paroxetine had to remain stable throughout the study unless a dose reduction was deemed necessary for management of an adverse event.

Patients taking SSRIs or SNRIs concomitantly were to be monitored for serotonin syndrome which includes altered mental status, hypertension, restlessness, myoclonus, hyperthermia, hyperreflexia, diaphoresis, shivering, and tremor.

Concomitant use of hypnotics at bedtime (e.g., eszopiclone, zolpidem, zaleplon, trazodone [up to 50 mg/day]) for the nighttime treatment of insomnia was allowed, provided the dose had been stable for at least 1 month prior to randomization and remained stable throughout the study.

In addition, concomitant use of short acting benzodiazepines (e.g., midazolam, oxazepam, low dose alprazolam [up to 0.5 mg/day]) for behavioral disturbances was allowed.

All other benzodiazepines were prohibited, except for lorazepam use for short-term treatment of agitation. Patients on lorazepam prior to study entry were to be on the same treatment regimen as allowed in the study (up to 1.5 mg/day and not to exceed 3 days in a 7-day period.

### 2.4.7.2. Rescue Medication for the Symptoms of Agitation

Patients could receive oral lorazepam as rescue medication for the short-term treatment of symptoms of agitation if deemed necessary by the Investigator. Lorazepam was to be administered in a dose up to 1.5 mg/day and not to exceed 3 days in a 7-day period. Caregivers were required to record concomitant use of lorazepam in the diary and were to be reminded of the potential increase in the risk of falling by benzodiazepines.

### 2.4.7.3. Prohibited Concomitant Medications

A list of examples of prohibited medications was provided in Appendix 1 of the protocol. These included ketoconazole, itraconazole, voriconazole, carbonic anhydrase inhibitors, amiodarone, cimetidine, diltiazem, verapamil, protease inhibitors (e.g., saquinavir, ritonavir, atazanavir, indinavir), macrolide antibiotics (e.g., erythromycin, telithromycin, clarithromycin, dirithromycin, roxithromycin), tricyclic antidepressants (e.g., imipramine, desipramine, amitriptyline, nortriptyline), quinidine, dextromethorphan (over-the-counter and prescription), quinine, mefloquine, St. John's wort, hyperforin, rifampicin, phenytoin, carbamazepine, oxcarbazepine, phenobarbital, cyproterone, thioridazine, trifluoperazine, chlorpromazine, promazine, perphenazine, methotrimeprazine, and fluphenazine.

Monoamine oxidase inhibitors (MAOIs) were prohibited throughout the study. Patients were required to allow at least 14 days after stopping study drug before starting an MAOI.

### 2.5. Efficacy and Safety Variables

### 2.5.1. Efficacy and Safety Measurements Assessed and Flow Chart

A schedule of study events is presented in Table 59.

TABLE 59
Study Schedule

| | Visit: | | | | | | | | | | |
|---|---|---|---|---|---|---|---|---|---|---|---|
| | | | Visit | Visit | Visit | Phone | Visit | Visit | Phone | Visit | Follow-up |
| | Screening ^{a} | Baseline | 2 ^{a} | 2.1 ^{a} | 3 ^{a,s} | Call ^{a,b,s} | 4 ^{a,s} | 5 ^{a} | Call ^{a,b} | 6 ^{a,d}/ET ^{c} | Visit ^{c} |
| | Study Day: | | | | | | | | | | |
| | Day −28 | | | | | | | | | | 30-days |
| | to −1 | Day 1 | Day 8 | Day 15 | Day 22 | Day 29 | Day 43 | Day 64 | Day 71 | Day 85 | Postdose |
| | End of Study Week: | | | | | | | | | | |
| | Week −4 | | | | | | | | | | |
| Procedure | to −1 | | Week 1 | Week 2 | Week 3 | Week 4 | Week 6 | Week 9 | Week 10 | Week 12 | |
| Sign informed consent | X | | | | | | | | | | |
| forms | | | | | | | | | | | |
| Medical history | X | | | | | | | | | | |
| Review of eligibility ^{e} | X | X | | | | | | | | | |
| Randomization | X | X | | | | | X | | | | |
| Physical and neurological | X | | | | | | X | | | X | |
| examination | | | | | | | | | | | |
| Vital signs and weight | X | X ^{f} | X | X | X | | X | X | | X ^{f} | |
| ADCS-CGIC-Overall | | X ^{g} | | | | | X | | | X | |
| CGIS-Agitation | X | X | | | | | X | | | X | |
| mADCS-CGIC-Agitation | | X ^{h} | | | | | X | | | X | X |
| Risk assessment for falls | X | | | | | | X ⁱ | | | X ⁱ | |
| (worksheet and TUG test) | | | | | | | | | | | |
| ECG | X ^{j} | X ^{k} | | X | X | | X | X | | X | |
| Adverse events | | X | X | X | X | X | X | X | X | X | X |
| Prior and concomitant: | X | X | X | X | X | X | X | X | X | X | X |
| medications, nondrug | | | | | | | | | | | |
| therapies, and | | | | | | | | | | | |
| nonpharmacological | | | | | | | | | | | |
| interventions for agitation | | | | | | | | | | | |
| MMSE | X | X | | | | | X | | | X | |
| GMHR | X | | | | | | | | | X | |
| CMAI | X | X | X | X | X | | X | X | | X | X |
| NPI | X ^{l} | X | X ^{l} | X ^{l} | X | | X | X | | X | |
| CSDD | X | | | | | | X | | | X | |
| ZBI | | X | | | | | X | | | X | |
| DEMQOL ^{m} | | X | | | | | X | | | X | |
| ADAS-cog ⁿ | | X | | | | | X | | | X | |
| PGIC ^{o} | | | | | | | X | | | X | |
| RUD | | X | | | | | X | | | X | |
| ESS ⁿ | | X | | | | | X | | | X | |
| S-STS | X | X | X | X | X | | X | X | | X | X |
| Administer morning dose of | | X | X ^{p} | X ^{p} | X | | X | X | | X | |
| study drug in-clinic | | | | | | | | | | | |
| Chemistry, hematology, and | X ^{q} | | | | X | | X | X | | X ^{q} | |
| urinalysis | | | | | | | | | | | |
| Urine pregnancy test ^{r} | X | X | | | | | X | | | X | |
| PK blood sample | | | | | | | X | | | X | |
| CYP2D6 blood sample | | X | | | | | | | | | |
| Dispense study drug and | | X | | | X | | X | X | | | |
| diary card | | | | | | | | | | | |
| Review and return unused | | | X ^{p} | X ^{p} | X | | X | X | | X | |
| study drug and diary card | | | | | | | | | | | |

ADAS-cog = Alzheimer's Disease Assessment Scale-cognitive subscale;
ADCS-CGIC-Overall = Alzheimer's Disease Cooperative Study-Clinical Global Impression of Change for Overall Clinical Status;
CGIS-Agitation = Clinical Global impression of Severity of Illness scale for Agitation;
CMAI = Cohen-Mansfield Agitation Inventory;
CSDD = The Cornell Scale for Depression in Dementia;
DEMQOL = Dementia Quality of Life scale;
ECG = electrocardiogram;
ESS = Epworth Sleepiness Scale;
ET = early termination;
GMHR = General Medical Health Rating;
mADCS-CGIC-Agitation = modified Alzheimer's Disease Cooperative Study-Clinical Global Impression of Change scale for Agitation;
MMSE = Mini Mental State Examination;
NPI = Neuropsychiatric Inventory;
PGIC = Patient Global Impression of Change rated by the caregiver;
PK = pharmacokinetics;
RUD = Resource Utilization in Dementia;
S-STS = Sheehan Suicidality Tracking Scale;
T3 = triiodothyronine;
T4 = thyroxine;
TSH = thyroid-stimulating hormone;
TUG = Timed Up and Go;
ZBI = Zarit Burden Interview
Note:
Whenever possible, each patient and caregiver was to have the rating scales administered by the same raters throughout the study, for consistency of ratings. The following scales HAD TO be administered by the same rater at each visit: CMAI, NPI, mADCS-CGIC-Agitation, and CGIS-Agitation.
^{a} Study visits had a +/−3-day window except Screening, Visit 2, and phone calls. Screening, Visit 2, and phone calls (excludes follow-up phone calls for ET patients) had a +3-day window. The Screening period could be extended after discussion with and approval by die Medical Monitor.
^{b}Phone calls were to be made to patient/caregiver to collect adverse events and query concomitant medication use.
^{c} ET visit for patients who withdrew prior to study completion. Patients who terminated early from the study received daily phone calls for 5 consecutive days following the ET visit to query on their overall well-being and an in-clinic Follow-up visit 30 days after last dose of study drug for selected safety and efficacy assessments.
^{d}Patients who did not roll over to the extension study (Study 15-AVP-786-303) were to receive a safety phone call 30 days after the last dose of study drug.
^{e} For each patient, a protocol eligibility form was completed,
^{f} Weight was to be measured only at the Baseline Visit and Visit 6.
^{g} The ADCS-CGIC-Overall Baseline evaluation worksheet was to be completed to record Baseline information for assessing change at Visits 4 and 6.
^{h} The mADCS-CGIC-Agitation Baseline evaluation worksheet was to be completed to record Baseline information for assessing change at Visits 4 and 6.
ⁱ Only the TUG test was to be performed for risk assessment of falls at Visits 4 and 6.
^{j} ECG was to be performed in triplicate at the Screening Visit.
^{k} ECG was to be performed predose and postdose.
^{l} Only the Agitation/Aggression domain of the NPI was to be performed at the Screening Visit, Visit 2, and Visit 2.1.
^{m} The proxy version was to be rated by the caregiver. The non-proxy version was to be rated only by patients with an MMSE score of ≥10 at Baseline.
ⁿ ADAS-cog and ESS were to be rated only by patients with an MMSE score of ≥10 at Baseline.
^{o} PGIC was to be rated by the caregiver.
^{p} The morning dose of study drug could have been administered at home if the visit was to occur within 2 hours of dosing; the time of dosing was to be noted by the patient/caregiver. The blister card and diary card were to be brought to the clinic and returned to the patient/caregiver after reviewing for compliance.
^{q} Thyroid function tests (TSH, and reflex T3 and T4 if TSH was abnormal) were to be performed at the Screening Visit. Glycosylated hemoglobin (HbA1c) test was to be performed at the Screening Visit and Visit 6.
^{r} Urine pregnancy test was to be performed for females of childbearing potential only.
^{s}A one-time downward dose adjustment was allowed after Visit 3 (Week 3) up to and including Visit 4 (Week 6), i.e. Day 23 to Day 43. Patients had to return to the clinic for an unscheduled visit for safety assessments.

### 2.5.1.1. Efficacy Endpoints

The efficacy endpoints included validated scales and questionnaires to assess changes in behaviors associated with agitation, depression, cognitive dysfunction, quality of life (QOL), and caregiver stress. Whenever possible, each patient and caregiver was to have the rating scales administered by the same raters throughout the study, for consistency of ratings. The following scales were required to be administered by the same rater at each visit: Cohen-Mansfield Agitation Inventory (CMAI), NPI, modified Alzheimer's Disease Cooperative Study-Clinical Global Impression of Change scale for Agitation (mADCS-CGIC-Agitation), and CGIS-Agitation score.

### 2.5.1.1.1. Primary Efficacy Assessments

The primary efficacy endpoint was the change from Baseline to Week 12 (Day 85) in the composite CMAI scores (CMAI Total score). The CMAI (long-form version) was used to assess the frequency of manifestations of agitated behaviors in elderly persons. It consists of 29 agitated behaviors that are further categorized into distinct agitation syndromes, also known as CMAI Factors of agitation. These distinct agitation syndromes include: Aggressive Behavior, Physically Non-aggressive Behavior, and Verbally Agitated Behavior. Scores for the 3 dimensions, or CMAI subscales, were derived based on the factor structure described by Rabinowitz J, Davidson M, De D P P, Katz I, Brodaty H, Cohen-Mansfield J. Factor analysis of the Cohen-Mansfield Agitation Inventory in three large samples of nursing home patients with dementia and behavioral disturbance. American Journal of Geriatric Psychiatry. 2005; 13(11):991-998 and described elsewhere herein were also assessed as secondary efficacy endpoints.

Each of the 29 items is rated on a 7-point scale of frequency (1=never, 2=less than once a week but still occurring, 3=once or twice a week, 4=several times a week, 5=once or twice a day, 6=several times a day, 7=several times an hour). The ratings are based on the 2 weeks preceding assessment of CMAI; a decrease in CMAI scores indicates improvement in the frequency of agitated behaviors. The CMAI Total score is calculated as the sum of ratings for all 29 items and ranges from 29 to 203.

The CMAI was assessed at Screening (Day −28 to Day −1), Baseline (Day 1), Day 8 (Visit 2), Day 15 (Visit 2.1), Day 22 (Visit 3), Day 43 (Visit 4), Day 64 (Visit 5), and Week 12 (Visit 6), and Follow-up visit (for ET patients; Table 59). The CMAI had to be administered by the same rater at each visit.

### 2.5.1.1.2. Key Secondary Efficacy Assessments

The key secondary efficacy endpoints were mADCS-CGIC-Agitation score at Week 12 and change from Baseline in CGIS-Agitation score at Week 12.
mADCS-CGIC-Agitation: The mADCS-CGIC-Agitation is a modification of the standard Alzheimer's Disease Cooperative Study—Clinical Global Impression of Change (ADCS-CGIC) instrument to better assess aspects relevant to studying agitation in Alzheimer's disease. It contains questions related to agitation and an assessment of the Clinician's Impression of Change focused specifically on agitation. It was originally designed for the Citalopram study for Agitation in Alzheimer's disease (CitAD) and utilizes a semi-structured interview of both patient and caregiver to determine a Baseline level of severity for agitation. Subsequent evaluations assess for change from Baseline and also utilize the semi-structured agitation interview of both patient and caregiver. The mADCS-CGIC-Agitation had to be administered by the same rater at each visit.
CGIS-Agitation: This is an observer-rated scale that measures illness severity and is one of the most widely used brief assessment tools in psychiatry research. The Early Clinical Drug Evaluation Unit (ECDEU) version of the CGIS is the most widely used format of this validated tool, and it asks that the clinician rate the patient relative to their past experience with other patients with the same diagnosis, with or without collateral information. The CGIS is a 7-point (1-7) scale (1=normal, not at all ill; 7=among the most extremely ill patients) and assesses severity of agitation in this study. The CGIS-Agitation had to be administered by the same rater at each visit.

### 2.5.1.1.3. Other Secondary Efficacy Assessments

The other secondary efficacy endpoints were NPI—Agitation/Aggression domain score and Caregiver Distress score, NPI—Aberrant Motor Behavior Domain score, Zarit Burden Interview (ZBI), NPI—Irritability/Lability domain score, Patient Global Impression of Change (PGIC), Dementia Quality of Life (DEMQOL), CSDD, Resource Utilization in Dementia (RUD), NPI Total score, ADCS-CGIC-Overall, Alzheimer's Disease Assessment Scale-cognitive subscale (ADAS-cog), and General Medical Health Rating (GMHR):
NPI—Agitation/Aggression domain score and Caregiver Distress score: The NPI is a validated clinical instrument for evaluating psychopathology in a variety of disease settings, including dementia. The NPI is a retrospective caregiver-informant interview covering 12 neuropsychiatric symptom domains: Delusions, Hallucinations, Agitation/Aggression, Depression/Dysphoria, Anxiety, Elation/Euphoria, Apathy/Indifference, Disinhibition, Irritability/Lability, Aberrant Motor Behavior, Sleep And Nighttime Behavioral Disorders, and Appetite/Eating Disorders. The scripted NPI interview includes a compound Screening question for each symptom domain, followed by a list of interrogatives about domain-specific behaviors that is administered when a positive response to a Screening question is elicited. Neuropsychiatric manifestations within a domain are collectively rated by the caregiver in terms of both frequency (1 to 4) and severity (1 to 3), yielding a composite symptom domain score (frequency×severity). Frequency and severity rating scales have defined anchor points to enhance the reliability of caregiver responses. Caregiver Distress is rated for each positive neuropsychiatric symptom domain on a scale anchored by scores of 0 (not distressing at all) to 5 (extremely distressing). The NPI domains are generally evaluated for behaviors within the preceding 4 weeks but can be modified according to the needs of the study; in this study, the recall period was 2 weeks for all the visits. The NPI nursing home version (NPI-NH) was used for patients from inpatient or assisted-living facilities. The questions in the NPI-NH were rephrased for professional caregivers who might not have known the patients prior to the onset of illness; however, the overall instrument domains and scoring were identical to the NPI except for the Caregiver Distress section, which was replaced with occupational disruptiveness in the NPI-NH version. The NPI had to be administered by the same rater at each visit. The Agitation/Aggression domain in the NPI was assessed as part of the NPI Total score.
NPI—Aberrant Motor Behavior Domain score (see above).
ZBI: This is a 22-item scale used to assess the impact of patient's disabilities on the caregiver's life. It is designed to reflect the burden experienced by caregivers of dementia patients and can either be completed by the caregiver or administered as an interview. It is the most commonly used scale for measuring burden in caregivers of patients with dementia and also other illnesses. The ZBI has been shown to have high internal-reliability with an estimated Cronbach's alpha at 0.88 and 0.91, and test-retest reliability at 0.71. Validity has been estimated by correlating the total score with a single global rating of burden (r=0.71) For each item of the scale, the caregiver has to indicate how often they felt that way (never, rarely, sometimes, quite frequently, or nearly always). The score ranges from 0 to 88 and is determined by adding the numbered responses of the individual items. Higher scores indicate greater Caregiver Distress.
NPI—Irritability/Lability domain (see above).
PGIC: This is a 7-point (1-7) scale used to assess treatment response, and it is rated as: very much improved, much improved, minimally improved, no change, minimally worse, much worse, or very much worse.
DEMQOL: This is a scale used to evaluate health-related QOL in patients with dementia and their caregivers. There are 2 versions of the DEMQOL, a 28-item version (rated by patient) and a 31-item version (DEMQOL-Proxy, rated by caregiver). Both the 28-item and 31-item versions are recommended to be used for evaluating patients (and their caregivers) with mild to moderate dementia (MMSE≥10). For patients with severe dementia, only the DEMQOL-Proxy (administered to caregiver) is used.
CSDD: This scale was specifically developed to assess signs and symptoms of major depression in patients with dementia. Because some of these patients may give unreliable reports, the CSDD uses a comprehensive interviewing approach that derives information from the patient and the caregiver. Information is elicited through 2 semi-structured interviews; an interview with a caregiver and an interview with the patient. The interviews focus on depressive symptoms and signs occurring during the week preceding the assessment. Each item is rated for severity on a scale of 0-2 (0=absent, 1=mild or intermittent, 2=severe). The item scores are added. Scores above 10 indicate a probable major depression, scores above 18 indicate a definite major depression, and scores below 6 as a rule are associated with absence of significant depressive symptoms.
RUD: The RUD is used to calculate healthcare costs associated with dementia. It evaluates dementia patients' utilization of formal and informal healthcare resources, including hospitalizations and doctor visits, living assistance, and time spent by nonprofessional caregivers. Within the context of clinical trials, the RUD is often used to determine the cost effectiveness of new pharmaceutical treatments. The RUD is administered as a semi-structured interview with the patient's primary caregiver and contains 2 sections; one focusing on caregiver impact (loss of work and leisure time incurred by caregiver) and the other focusing on the patient's use of healthcare resources. The total healthcare costs associated with the patient's dementia can be estimated by multiplying the number of units used (e.g., hours of caregiver time, visits to doctors, nights in accommodation) by the corresponding unit price vector.
NPI Total score (see above).
ADCS-CGIC-Overall: This scale is to provide a means to reliably assess change from a Baseline level of global function within the timeframe of a clinical trial. Unlike a targeted symptom scale, the ADCS-CGIC-Overall takes into account a patient's overall function in the cognitive, behavioral, and functional activity domains. Relying on information gathered through a semi-structured interview of the patient and caregiver, the ADCS-CGIC-Overall focuses on clinician's observations of change in the patient's cognitive, functional, and behavioral performance since the beginning of a trial. Once the Baseline level of severity is established, the change score at the Follow-up visits is based on information gathered from the patient and caregiver interviews. The ADCS-CGIC-Overall is rated as: marked improvement, moderate improvement, minimal improvement, no change, minimal worsening, moderate worsening, or marked worsening.
ADAS-cog: The ADAS was designed to evaluate the cognitive and non-cognitive behavioral dysfunction characteristics of patients with Alzheimer's disease. The cognitive subscale (ADAS-cog) consists of 11 subsets related to memory, praxis, and language. The ADAS-cog was assessed for patients with an MMSE score of ≥10 at the Baseline visit.
GMHR: This is a global clinical rating for medical health, designed to quantify in a single number (1 to 4) the severity of general comorbidity in a patient with dementia. A rating of 1=poor, 2=fair, 3=good, and 4=excellent to very good.

### 2.5.1.2. Safety Endpoints

The safety endpoints evaluated were treatment-emergent adverse events (TEAEs), physical and neurological examinations, vital signs, clinical laboratory tests, ECGs, S-STS, MMSE, Timed Up and Go (TUG) Test, and the Epworth Sleepiness Scale (ESS).

### 2.5.1.2.1. Safety Assessments

### 2.5.1.2.1.1. Adverse Events

Caregivers were to be queried regarding adverse events at each clinic visit after the Screening Visit (Table 59) and at the safety phone calls at Days 29 and 71. All reported adverse events were to be assessed and recorded. Any adverse event newly reported after receiving the last dose of study drug and up until 30 days after receiving the last dose of study drug was to be followed up until 30 days.

The severity of each adverse event was to be graded on a 3-point scale (mild, moderate, or severe) and reported in detail as indicated on the eCRF. The relationship of each adverse event to study drug was to be determined by the Investigator as not related, unlikely related, possibly related, or related.

### 2.5.1.2.1.2. Physical and Neurological Examinations

Physical and neurological examinations were to be performed at the timepoints indicated in Table 59. The physical examination was to include assessments of head, eyes, ears, nose, throat, lymph nodes, skin, extremities, respiratory, gastrointestinal, musculoskeletal, cardiovascular, and nervous systems. The neurological examination was to include assessments of mental status, cranial nerves, motor system, reflexes, coordination, gait and station, and sensory system. The physical and neurological examinations were to be performed by the same person each time, whenever possible. Any clinically significant changes in physical and neurological examination findings relative to the Screening examination were to be recorded as adverse events.

### 2.5.1.2.1.3. Vital Signs

Orthostatic blood pressure (BP) and heart rate (HR) measurements were to be performed at all clinic visits. Supine BP and HR were to be measured after the patient had rested for at least 5 minutes in the supine position. Each measurement was to be taken twice in the same position and recorded. After the measurement of supine BP and HR, the patient was to stand still for up to 3 minutes and a single measurement of standing BP and HR was to be recorded within these 3 minutes of standing.

Respiratory rate (breaths/minute) and body temperature (° F.) were to be assessed at all clinic visits. Weight was to be recorded at Baseline (Day 1) and Week 12 (Visit 6).

### 2.5.1.2.1.4. Clinical Laboratory Tests

The following clinical laboratory assessments were to be performed at the timepoints indicated in Table 59:
Blood chemistry (calcium, magnesium, phosphorus, glucose, sodium, potassium, chloride, carbon dioxide, blood urea nitrogen [BUN], serum creatinine, uric acid, albumin, total bilirubin, alkaline phosphatase, lactate dehydrogenase [LDH], aspartate aminotransferase [AST; previously called serum glutamic oxaloacetic transaminase], alanine aminotransferase [ALT; previously called serum glutamic pyruvic transaminase], creatine kinase [CK], gamma-glutamyl transferase [GGT], triglycerides, total protein, and total cholesterol)
Hematology (red blood cell [RBC] count, hemoglobin, hematocrit, white blood cell [WBC] count, neutrophils, bands, lymphocytes, monocytes, eosinophils, basophils, platelet count, and morphology)
Urinalysis (pH, specific gravity, protein, glucose, ketones, blood, leucocyte esterase, nitrates, and microscopic appearance)
Thyroid function tests (thyroid-stimulating hormone [TSH], and reflex triiodothyronine [T3] and thyroxine [T4] if TSH was abnormal) at Screening Visit only
Glycosylated hemoglobin (HbA1c) test at the Screening Visit and Visit 6 only

Any patients with clinically significant abnormal laboratory test results could have been required by the Medical Monitor to have a repeat test 1 week later or earlier, if medically indicated. Clinically significant laboratory abnormalities could have been a basis for exclusion from study entry.

### 2.5.1.2.1.5. Electrocardiograms

A resting 12-lead ECG was to be performed at the timepoints indicated in Table 59. At Screening, ECG was to be performed in triplicate. At Baseline (Day 1), 2 ECGs were to be performed; one prior to study drug dosing and one 2 to 3 hours after dosing. ECG equipment was provided by the central reader. ECG data were recorded at the study center and included general findings, HR (beats/minute), QRS complex, and PR and QTc intervals (milliseconds). Results were to be provided by the central reader to the Investigators within 24 hours.

### 2.5.1.2.2. Sheehan Suicidality Tracking Scale

The S-STS is a prospective scale that assesses treatment-emergent suicidal thoughts and behaviors and was to be assessed at the timepoints indicated in Table 59. Any change in the S-STS score indicating the presence of suicidality was to be evaluated by the Investigator and reported to the Medical Monitor.

### 2.5.1.2.3. Mini Mental State Examination

The MMSE is a brief 30-point questionnaire test that is used to screen for cognitive impairment and was to be assessed at the timepoints indicated in Table 59.

### 2.5.1.2.4. Timed Up and Go Test

The TUG test measures the time (in seconds) taken for an individual to stand up from a standard armchair, walk 3 meters, turn, walk back to the chair, and sit down; the test was to be assessed at the timepoints indicated in Table 59.

### 2.5.1.2.5. Epworth Sleepiness Scale

The ESS is an 8-item questionnaire that is used to measure sleepiness by rating the probability of falling asleep on 8 different situations that most people engage in during the day. The questions are rated on a 4-point scale (0 to 3) where 0=would never doze, 1=slight chance of dozing, 2=moderate chance of dozing, and 3=high chance of dozing. A total score of 0 to 9 is considered to be normal. The test was to be assessed at the timepoints indicated in in Table 59.

### 2.5.1.3. Pharmacokinetic Assessments

At Day 43 (Visit 4) and Week 12 (Visit 6), patients were to have a blood sample collected between 0 and 3 hours after the morning dose of study drug for analysis of plasma levels of d6-DM, d6-DM metabolites, and Q. The time when the patient was administered the dose of study drug and the time of the blood draw were to be recorded. Plasma samples were separated by centrifugation and frozen at −20° C. until assayed at the analytical unit.

### 2.6. Statistical Methods Planned in the Protocol and Determination of Sample Size

### 2.6.1. Statistical and Analytical Plans

Key details are summarized below.

### 2.6.1.1. Analysis Populations

Because Protocol Amendment 4, adding the AVP-786-42.63 cohort, substantively changed the study design, patients were considered to be in 2 separate cohorts: Cohort 1 included all patients randomized before Amendment 4, and Cohort 2 included all patients randomized after Amendment 4. Most analyses were performed in all patients (Cohorts 1 and 2 combined) and separately in Cohort 2 only. Definitions and statistical analyses, including the dataset definitions below, were applied to the 2 sets of analyses in the same fashion.

There were 3 analysis populations. Safety, Intent-to-treat (ITT), and Modified intent-to-treat (mITT), which are defined below.
The Safety Population includes all patients who received at least 1 dose of study drug. The Safety Population was used for all analyses of safety data. Patients were included in the treatment group based on the actual treatment received.
The ITT Population includes all patients who were randomized in the double-blind treatment period. The ITT Population was used for sensitivity analyses. Patients were included in the treatment group to which they had been randomized regardless of treatment received.
The mITT Population includes all patients randomized in the double-blind treatment period who received at least 1 dose of double-blind study drug, and had a Baseline and at least one postbaseline CMAI Total score assessment. The mITT Population was used for all efficacy analyses.

### 2.6.1.2. Efficacy

### 2.6.1.2.1. Primary Efficacy Endpoint Analysis Methods

### 2.6.1.2.1.1. Primary Analysis

The primary efficacy endpoint (change from Baseline to Week 12 in the CMAI Total score) was analyzed using a likelihood-based mixed model repeated measures (MMRM) analysis. This model was run on the mITT Population, using observed data.

The MMRM model included terms for treatment, cohort, visit, treatment-by-visit interaction, Baseline CMAI Total score, Baseline-by-visit interaction, Baseline NPI—Agitation/Aggression (≤6 vs >6), risk assessment for falls (normal/mild vs moderate/severe), concomitant use of antipsychotic medications (yes vs no).

An unstructured covariance matrix was used. If there had been convergence problems, then the use of first-order autoregressive (AR1) and/or the compound symmetry (CS) covariance matrices was to be considered and the first covariance structure converging to the best fit would have been used as the primary analysis.

Model estimates (treatment difference and its 95% confidence interval [CI]) are reported in addition to the p-value. Multiple comparison family-wise error rate (FWE) control is specified elsewhere herein.

### 2.6.1.2.1.2. Multiplicity

The FWE was controlled by testing the difference between the average treatment effect of AVP-786-28 and AVP-786-42.63 versus placebo first, at significant level 2-sided α=0.05. If this global test was significant, and the estimated treatment difference was in the predicted direction favoring the active treatment, then comparisons for each treatment group (AVP-786-28 and AVP-786-42.63) versus placebo were to be performed at a 2-sided α=0.05 level. For the primary efficacy endpoint comparisons (Family 1), a treatment group comparison is significant at 2-sided α=0.05 FWE level if both the global test and the test of that group comparison are significant at 2-sided α=0.05. If the global test and the comparison for each AVP-786 group versus placebo were all significant at 2-sided α=0.05, then the same procedure was to be repeated for the first key secondary efficacy endpoint (mADCS-CGIC-Agitation) comparisons (Family 2), at 2-sided α=0.05. If the global test and the comparison for each AVP-786 group versus placebo were all significant at 2-sided α=0.05, then the same procedure was to be repeated for the secondary key secondary efficacy endpoint (CGIS-Agitation) comparisons (Family 3), at 2-sided α=0.05.

### 2.6.1.2.1.3. Sensitivity Analyses

The MMRM assumes data are missing at random (MAR), which is a reasonable assumption in longitudinal clinical trials. However, the possibility of “missing not at random” (MNAR) data can never be ruled out. As sensitivity analyses for the MAR assumption, analyses for MNAR were carried out using Pattern Mixture Models (PMM) based on Multiple Imputation (MI) with mixed missing data mechanisms to investigate the response profile of dropout patients by last dropout reason under MNAR mechanism for the following 2 scenarios:
1. Dropout reasons due to adverse event as MNAR
2. All dropouts as MNAR

### 2.6.1.2.2. Secondary Efficacy Endpoint Analyses

The MMRM described above was used for analysis of the secondary efficacy endpoints (except RUD and GMHR) for the mITT Population. Note that the raw score at postbaseline visits for mADCS-CGIC-Agitation, ADCS-CGIC-Overall, and PGIC measure change from their corresponding Baseline. In the analyses for these endpoints, Baseline CMAI Total score was used as the covariate.

The sensitivity analysis described above for the primary endpoint was also performed for the secondary endpoints. An additional sensitivity analysis was performed on the change from Baseline in the CMAI Total score at Week 12 using analysis of covariance (ANCOVA) on the mITT Population. The model included factors of treatment, Baseline NPI—Agitation/Aggression (≤6 vs >6), risk assessment for falls (normal/mild vs moderate/severe), concomitant use of antipsychotic medications (yes vs no), and Baseline CMAI Total score as covariates. Missing data were imputed within a study stage using a last observation carried forward (LOCF) approach and a worst observation carried forward (WOCF)+LOCF approach. In the WOCF+LOCF approach, values that were missing due to lack of efficacy were imputed by WOCF, and values missing for any other reason were imputed by LOCF. Summary statistics including the change and percent change from Baseline, model estimates (least-square [LS] mean difference, 95% CI, and p-value), and the standard effect size (SES) are reported in addition to the p-value.

Finally, the primary endpoint was analyzed using the ITT Population in both the MMRM and ANCOVA models described above for the mITT Population.

### 2.6.1.2.2.1. Response Analysis

The number and percentage of patients who had favorable treatment response according to the CMAI Total score and the NPI—Agitation/Aggression domain score were summarized using the mITT Population. The following categories were used to classify response patients:
Response: Patients with a 30% reduction in the CMAI Total score.
Response: Patients with a 50% reduction in the CMAI Total score.
Response: Patients with a 30% reduction in the NPI—Agitation/Aggression domain score.
Response: Patients with a 50% reduction in the NPI—Agitation/Aggression domain score.
Response: Patients with score of 1 or 2 (marked improvement or moderate improvement) in mADCS-CGIC-Agitation.
Response: Patients with score of 1 or 2 (very much improved or much improved) in PGIC.

The number and percentage of response is provided by treatment group. Treatment effects were tested using a general estimating equation (GEE) model with the same effects used in the MMRM model.

### 2.6.1.2.2.2. Resource Utilization in Dementia and General Medical Health Rating Analysis

Descriptive analyses of the RUD variables are provided at Baseline, Week 6, and Week 12. Descriptive analyses of the GMHR variable will be provided at Baseline (Screening Visit) and Week 12.

### 2.6.1.2.3. Subgroup Analyses

Due to potential small sample sizes, the primary efficacy endpoint was analyzed for the below subgroups using ANCOVA model with missing data imputed by LOCF to evaluate potential differential treatment effect. The following subgroups were analyzed:
1. Baseline use of psychotropic medications based on CYP2D6 substrate status. Drugs that are major CYP2D6 substrates that could be counted in this analysis were aripiprazole, risperidone, duloxetine, fluoxetine, fluvoxamine, mirtazapine, paroxetine, and venlafaxine.
2. Baseline use of beta blocker medications based on CYP2D6 substrate status. Drugs that are major CYP2D6 substrates that could be counted in this analysis were carvedilol, metoprolol, propranolol, and timolol.
3. CMAI Factor 1 agitated patients, defined as patients who met the protocol inclusion requirement for CGIS-Agitation score of ≥4 and the criteria for CMAI Factor 1 Aggressive Behavior agitated status (CMAI manual) at Screening and Baseline. Based on CMAI manual, Factor 1 agitated status was defined by satisfying one of the following conditions:
   ≥1 aggressive behaviors occurring several times per week (score 4 or above), or
   ≥2 aggressive behaviors occurring once or twice per week (score 3 or above), or
   ≥3 aggressive behaviors occurring less than once per week (score 2 or above).
4. Patients who did not use antipsychotics at Baseline.
5. Additional Subgroups: Additional analyses of subgroups, such as age group, sex, and Baseline stratification factors, could have been performed for the primary efficacy endpoint if deemed important and sample size permitted.

### 2.6.1.3. Safety

Descriptive statistics and by-patient listings are presented for safety assessments, including TEAEs, clinical laboratory assessments, ECGs, vital signs, physical and neurological examinations, S-STS, MMSE, TUG test, and ESS. All safety analyses will be completed on the Safety Population.

### 2.6.2. Determination of Sample Size

Power calculation was performed assuming a normal distribution for the primary efficacy endpoint. For this 12-week study, the treatment effect size (AVP-786 vs. placebo) was assumed to be −0.42. A sample size of 140/arm was planned to provide 90/6 power with 2-sided α=0.05, allowing for a dropout and non-evaluable rate of 15% during the study.

Protocol Amendment 4 added the second active treatment group (AVP-786-42.63), with 140 additional patients, when approximately 100 patients have already been enrolled (50 AVP-786-28/4.9 patients and 50 placebo patients) in the study. To maintain an approximate 60% chance for an incoming patient to receive active drug, the sample size for the placebo treatment group was changed to 190. Overall, the total sample size of 470 patients was to include 140 patients each for AVP-786-28 and AVP-786-42.63 groups and 190 patients in the placebo group. This sample size would provide approximately 90% power to detect a treatment effect size of −0.42 between AVP-786-42.63 and the concurrent placebo treatment group.

### 3. Study Patients

### 3.1. Disposition of Patients

Of the 925 patients who were screened for the study, 522 (56.4%) patients were randomized to treatment (Table 60). The most common reason for screen failure was not meeting the eligibility criteria (314 screened patients, 33.9%), other (47 patients, 5.1%), and withdrew consent (32 patients, 3.5%).

Of the 522 patients randomized to treatment, 521 patients received at least 1 dose of study drug; 1 patient in the AVP-42.63 group discontinued the study due to a protocol deviation (did not have a CSDD assessment at Baseline) before receiving treatment.

The majority of patients completed the study (87.9%). A total of 63 patients (12.1%) discontinued from the study early. The most common reasons for early discontinuation overall were patient withdrawal by parent or guardian (3.3%), TEAEs (2.7%), and withdrawal by patient (2.1%). A higher percentage of patients in the AVP-786-28 group discontinued from the study early compared to the placebo and AVP-786-42.63 groups (8.6%, 19.9%, and 9.3% for the placebo, AVP-786-28, and AVP-786-42.63 groups, respectively).

TABLE 60
Overall Patient Disposition (All Patients)

| | Placebo | AVP-786-28 | AVP-786-42.63 | All Patients |
|---|---|---|---|---|
| | (N = 210) | (N = 151) | (N = 161) | (N = 925) |
| | n (%) | n (%) | n (%) | n (%) |
| Patients screened | — | — | — | 925 |
| Screen failures | — | — | — | 403 (43.6) |
| Adverse event | — | — | — | 4 (0.4) |
| Inclasion/exclusion criterion met | — | — | — | 314 (33.9) |
| Lost to follow-up | — | — | — | 6 (0.6) |
| Withdrew consent | — | — | — | 32 (3.5) |
| Other | — | — | — | 47 (5.1) |
| Patients randomized | 210 | 151 | 161 | 522 |
| Randomized patients who did not receive | 0 | 0 | 1 (0.6) | 1 (0.2) |
| study drug | | | | |
| Completed study | 192 (91.4) | 121 (80.1) | 146 (90.7) | 459 (87.9) |
| Patients who discontinued from study | 18 (8.6) | 30 (19.9) | 15 (9.3) | 63 (12.1) |
| Adverse event | 2 (1.0) | 6 (4.0) | 6 (3.7) | 14 (2.7) |
| Death | 0 | 2 (1.3) | 0 | 2 (0.4) |
| Lack of efficacy | 1 (0.5) | 0 | 0 | 1 (0.2) |
| Lost to follow-up | 1 (0.5) | 1 (0.7) | 1 (0.6) | 3 (0.6) |
| Non-compliance with study drug | 2 (1.0) | 0 | 1 (0.6) | 3 (0.6) |
| Physician decision | 1 (0.5) | 1 (0.7) | 1 (0.6) | 3 (0.6) |
| Pregnancy | 0 | 0 | 0 | 0 |
| Protocol deviation | 3 (1.4) | 0 | 1 (0.6) | 4 (0.8) |
| Study patient withdrawal by parent or | | | | |
| guardian | 3 (1.4) | 12 (7.9) | 2 (1.2) | 17 (3.3) |
| Study terminated by Sponsor | 0 | 0 | 0 | 0 |
| Trial site terminated by Sponsor | 0 | 0 | 0 | 0 |
| Withdrawal by patient | 4 (1.9) | 4 (2.6) | 3 (1.9) | 11 (2.1) |
| Other | 1 (0.5) | 4 (2.6) | 0 | 5 (1.0) |

Note:
Denominators for screen failures and reasons for screen failures are the number of patients screened.
Denominators for all other categories are the number of patients randomized in each group.

### 3.2. Protocol Deviations

Important protocol deviations are summarized in Table 61 (Safety Population). Overall, 43.0% patients had at least 1 important protocol deviation. The most frequently reported important protocol deviation in both stages was rater change (17.6%, 31.8%, and 20.6% patients in the placebo, AVP 786 28, and AVP 786 42.63 groups, respectively). Stratification deviations were reported for 9.5%, 4.6%, and 10.0% patients, respectively. Overall, important protocol deviations did not indicate any issues in interpretation of the data or any additional risk for the patients.

TABLE 61
Important Protocol Deviations (Safety Population)

| | Placebo | AVP-786-28 | AVP-786-42.63 | All Patients |
|---|---|---|---|---|
| | (N = 210) | (N = 151) | (N = 168) | (N = 521) |
| Important Protocol Deviation | n (%) | n (%) | n (%) | n (%) |
| Patients with any protocol deviation | 80 (38.1) | 79 (52.3) | 65 (40.6) | 224 (43.0) |
| Rater change | 37 (17.6) | 48 (31.8) | 33 (20.6) | 118 (22.6) |
| Stratification | 20 (9.5) | 7 (4.6) | 16 (10.0) | 43 (8.3) |
| Rescue therapy | 8 (3.8) | 3 (2.0) | 1 (0.6) | 12 (2.3) |
| LAR consent | 6 (2.9) | 3 (2.0) | 1 (0.6) | 10 (1.9) |
| Missing assessments | 3 (1.4) | 4 (2.6) | 3 (1.9) | 10 (1.9) |
| Consent | 2 (1.0) | 6 (4.0) | 1 (0.6) | 9 (1.7) |
| Males with QTeF > 450 msec | 3 (1.4) | 2 (1.3) | 1 (0.6) | 6 (1.2) |
| Females with QTcF > 470 msec | | | | |
| Hypersensitivity to study medication | 1 (0.5) | 2 (1.3) | 3 (1.9) | 6 (1.2) |
| Unqualified rater | 1 (0.5) | 0 | 5 (3.1) | 6 (1.2) |
| Wrong treatment | 1 (0.5) | 2 (1.3) | 3 (1.9) | 6 (1.2) |
| Unstable hypnotics therapy (at Baseline) | 1 (0.5) | 3 (2.0) | 1 (0.6) | 5 (1.0) |
| Prohibited concomitant medications (on treatment) | 3 (1.4) | 1 (0.7) | 1 (0.6) | 5 (1.0) |
| Agitation secondary to AD therapy dose change (on | 2 (1.0) | 3 (2.0) | 0 | 5 (1.0) |
| treatment) | | | | |
| Rescue therapy other than lorazepam | 4 (1.9) | 0 | 1 (0.6) | 5 (1.0) |
| AD diagnosis (at Baseline) | 1 (0.5) | 2 (1.3) | 1 (0.6) | 4 (0.8) |
| Unstable antidepressant therapy (at Baseline) | 1 (0.5) | 1 (0.7) | 2 (1.3) | 4 (0.8) |
| Unstable agitation secondary to AD therapy (at | 2 (1.0) | 1 (0.7) | 1 (0.6) | 4 (0.8) |
| Baseline) | | | | |
| Agitation secondary to AD new therapy (on treatment) | 3 (1.4) | 0 | 1 (0.6) | 4 (0.8) |
| Hypnotics therapy dose change (on treatment) | 2 (1.0) | 1 (0.7) | 1 (0.6) | 4 (0.8) |
| Unstable concomitant AD therapy (at Baseline) | 1 (0.5) | 2 (1.3) | 0 | 3 (0.6) |
| Benzodiazepine that is not short-acting (at Baseline) | 3 (1.4) | 0 | 0 | 3 (0.6) |
| Concurrent clinical study | 0 | 2 (1.3) | 1 (0.6) | 3 (0.6) |
| History of complete heart block, QTc prolongation, or | 1 (0.5) | 0 | 1 (0.6) | 2 (0.4) |
| torsades de pointes | | | | |
| AD therapy dose new therapy (on treatment) | 1 (0.5) | 0 | 1 (0.6) | 2 (0.4) |
| Antidepressant therapy (on treatment) | 0 | 0 | 2 (1.3) | 2 (0.4) |
| Caregiver consent | 0 | 1 (0.7) | 0 | 1 (0.2) |
| Agitation torsades des pointes | 0 | 1 (0.7) | 0 | 1 (0.2) |
| Clinically significant ECG findings (at Baseline) | 0 | 0 | 1 (0.6) | 1 (0.2) |
| Alprazolam >= 0.5 mg/day (at Baseline) | 0 | 0 | 1 (0.6) | 1 (0.2) |
| History of substance and/or alcohol abuse | 0 | 0 | 1 (0.6) | 1 (0.2) |
| AD therapy dose change (on treatment) | 0 | 0 | 1 (0.6) | 1 (0.2) |
| Randomization | 1 (0.5) | 0 | 0 | 1 (0.2) |

AD = Alzheimer's disease;
ECG = electrocardiogram;
QTc = QT corrected;
QTcF = QT corrected using Fridericia's method

### 4. Efficacy Evaluation

### 4.1. Data Sets Analyzed

Analysis sets are summarized for both cohorts combined in Table 62. All 522 randomized patients were included in the ITT Population, and 519 randomized patients were included in the mITT Population. One patient in the AVP-786-28 group and 2 patients in the AVP-786-42.63 group were excluded from the mITT population because they did not have at least one postbaseline efficacy assessment.

The Safety Population included a total of 521 of 522 randomized patients.

TABLE 62
Summary of Analysis Populations (All Randomized Patients)

| Analysis Population/Subset, n | Placebo | AVP-786-28 | AVP-786-42.63 | All Patients |
|---|---|---|---|---|
| Cohort 1 and Cohort 2 | 210 | 151 | 161 | 522 |
| Randomized | | | | |
| Study populations | 210 | 150 | 159 | 519 |
| Modified Intent-to-Treat (mITT) | | | | |
| Intent-to-Treat (ITT) | 210 | 151 | 161 | 522 |
| Safety | 210 | 151 | 160 | 521 |

ITT = intent-to-treat;
mITT = modified intent-to-treat
Note:
Protocol Amendment 4 resulted in AVP-786-42.63 added in 2:3:3 ratio to receive AVP-786-28:AVP-786-42.63:Placebo.

### 4.2. Demographic and Other Baseline Characteristics

In general, the treatment groups were well balanced with regard to sex (56.9% were female overall), race (92.0% white, 6.3% black), ethnicity (38.9% not Hispanic or Latino), and age (median 76 years overall; Table 63).

The patient's spouse (41.7%) and child (25.5%) were most frequently reported as being the patient's caregiver, and the majority of patients were outpatients (91.7%). There did not appear to be any important between-group differences in terms of patient caregiver or living arrangements.

TABLE 63
Demographics and Baseline Characteristics (All Randomized Population)

| | Placebo | AVP-786-28 | AVP-786-42.63 | All Patients |
|---|---|---|---|---|
| Characteristics | (N = 210) | (N = 151) | (N = 161) | (N = 522) |
| Sex n (%) | 210 | 151 | 161 | 52.2 |
| Female | 117 (55.7) | 84 (55.6) | 96 (59.6) | 297 (56.9) |
| Male | 93 (44.3) | 67 (44.4) | 65 (40.4) | 225 (43.1) |
| Race n (%) | 210 | 151 | 161 | 522 |
| White | 196 (93.3) | 129 (85.4) | 155 (96.3) | 480 (92.0) |
| Black or African American | 13 (6.2) | 14 (9.3) | 6 (3.7) | 33 (6.3) |
| Asian | 0 | 2 (1.3) | 0 | 2 (0.4) |
| American Indian or Alaska Native | 1 (0.5) | 0 | 0 | 1 (0.2) |
| Native Hawaiian or Other Pacific | 0 | 1 (0.7) | 0 | 1 (0.2) |
| Islander | | | | |
| Other | 0 | 5 (3.3) | 0 | 5 (1.0) |
| Ethnicity n (%) | 210 | 151 | 161 | 522 |
| Hispanic or Latino | 128 (61.0) | 78 (51.7) | 113 (70.2) | 319 (61.1) |
| Not Hispanic or Latino | 82 (39.0) | 73 (48.3) | 48 (29.8) | 203 (38.9) |
| Age (years) | 210 | 151 | 161 | 522 |
| Mean (SD) | 76.7 (8.1) | 74.6 (7.9) | 74.8 (7.3) | 75.5 (7.9) |
| Median | 78.0 | 76.0 | 75.0 | 76.0 |
| Min, Max | 50, 90 | 52, 90 | 56, 90 | 50, 90 |
| Age group (years) n (%) | 210 | 151 | 161 | 522 |
| <65 | 17 (8.1) | 14 (9.3) | 14 (8.7) | 45 (8.6) |
| ≥65 | 193 (91.9) | 137 (90.7) | 147 (91.3) | 477 (91.4) |
| Weight (kg) | 210 | 151 | 160 | 521 |
| Mean (SD) | 73.26 (15.52) | 73.76 (15.23) | 72.47 (16.29) | 73.16 (15.66) |
| Median | 73.10 | 73.00 | 70.88 | 72.45 |
| Min, Max | 33.8, 125.1 | 37.8, 115.2 | 34.7, 171.0 | 33.8, 171.0 |

SD = standard deviation
Note:
Denominators are the number of patients who had that parameter assessed.

Mean (standard deviation [SD]) CMAI Total scores at Baseline were similar between treatment groups (Table 64). The mean (SD) CMAI Total score for all patients was 71.5 (20.62). However, a higher percentage of patients in the placebo group were “agitated” based on the CMAI Aggressive Behavior Score (79.05%, 65.56%, and 64.60% for placebo, AVP-786-28, and AVP-786-42.63, respectively). Baseline means across treatment groups were also similar across groups for the NPI Total score, NPI—Agitation/Aggression domain score, and CGIS-Agitation score.

At Baseline, 87.1% patients were taking at least one medication to treat Alzheimer's disease, and 28.2% patients were taking at least one medication to treat agitation. A higher proportion of patients in the placebo group than in the active treatment groups was taking medication for agitation at Baseline (31.0%, 26.5%, and 26.3% for placebo, AVP-786-28, and AVP-786-42.63, respectively), but the difference did not appear to be due to any class of medications.

TABLE 64
Baseline Efficacy Assessments (All Randomized Population)

| | Placebo | AVP-786-28 | AVP-786-42.63 | All Patients |
|---|---|---|---|---|
| Assessment Statistics | (N = 210) | (N = 151) | (N = 161) | (N = 522) |
| CMAI-Total score | 210 | 151 | 160 | 521 |
| Mean (SD) | 73.7 (21.13) | 68.8 (19.32) | 71.1 (20.94) | 71.5 (20.62) |
| Median | 69.0 | 65.0 | 67.0 | 67.0 |
| Min, Max | 34, 139 | 36, 160 | 33, 155 | 33, 160 |
| CMAI-Aggressive Behavior | 210 | 151 | 160 | 521 |
| score | | | | |
| Mean (SD) | 21.0 (7.59) | 19.3 (7.80) | 19.6 (8.59) | 20.1 (7.99) |
| Median | 19.0 | 17.0 | 17.0 | 18.0 |
| Min, Max | 12, 54 | 12, 63 | 12, 65 | 12, 65 |
| CMAI-Aggressive Behavior | 210 | 151 | 160 | 521 |
| n (%) | | | | |
| Agitated | 166 (79.05) | 99 (65.56) | 104 (64.60) | 369 (70.69) |
| Not Agitated | 44 (20.95) | 52 (34.44) | 56 (34.78) | 152 (29.12) |
| CMAI-Physically Non- | 210 | 151 | 160 | 521 |
| aggressive Behavior score | | | | |
| Mean (SD) | 21.0 (7.99) | 19.6 (7.60) | 20.4 (7.53) | 20.4 (7.74) |
| Median | 20.0 | 19.0 | 20.0 | 20.0 |
| Min, Max | 6, 42 | 6, 39 | 6, 37 | 6, 42 |
| CMAI-Physically Non- | 210 | 151 | 160 | 521 |
| aggressive Behavior n (%) | | | | |
| Agitated | 187 (89.05) | 132 (87.42) | 140 (86.96) | 459 (87.93) |
| Not Agitated | 23 (10.95) | 19 (12.58) | 20 (12.42) | 62 (11.88) |
| CMAI-Verbally Agitated | 210 | 151 | 159 | 520 |
| Behavior score | | | | |
| Mean (SD) | 17.4 (5.89) | 16.3 (5.15) | 17.4 (5.50) | 17.1 (5.58) |
| Median | 17.0 | 16.0 | 17.0 | 17.0 |
| Min, Max | 4, 28 | 5, 28 | 7, 28 | 4, 28 |
| CMAI-Verbally Agitated | 210 | 151 | 159 | 520 |
| Behavior n (%) | | | | |
| Agitated | 192 (91.43) | 136 (90.07) | 149 (92.55) | 477 (91.38) |
| Not Agitated | 18 (8.57) | 15 (9.93) | 10 (6.21) | 43 (8.24) |
| NPI-Total score | 210 | 151 | 160 | 521 |
| Mean (SD) | 37.9 (19.94) | 34.3 (19.04) | 39.0 (19.13) | 37.2 (19.49) |
| Median | 35.0 | 31.0 | 37.0 | 35.0 |
| Min, Max | 6, 115 | 2, 99 | 3, 144 | 2, 144 |
| NPI AA domain score | 210 | 151 | 160 | 521 |
| Mean (SD) | 6.8 (2.41) | 6.5 (2.36) | 7.0 (2.05) | 6.8 (2.29) |
| Median | 6.0 | 6.0 | 6.0 | 6.0 |
| Min, Max | 1, 12 | 0, 12 | 3, 12 | 0, 12 |
| CGIS-Agitation score | 210 | 151 | 160 | 521 |
| Mean (SD) | 4.4 (0.58) | 4.4 (0.57) | 4.4 (0.54) | 4.4 (0.56) |
| Median | 4.0 | 4.0 | 4.0 | 4.0 |
| Min, Max | 4, 6 | 4, 7 | 4, 6 | 4, 7 |

CMAI = Cohen-Mansfield Agitation Inventory;
NPI AA = Neuropsychiatric Inventory Agitation/Aggression;
CGIS-Agitation = Clinical Global Impression of Severity of Illness scale for Agitation;
mITT = modified intent-to-treat;
SD = standard deviation

### 4.3. Measurements of Treatment Compliance

Overall compliance was good across all treatment groups. Mean compliance in the Safety Population was 99.4%, and 95.6% patients were 80% to 120% compliant (Table 65).

TABLE 65
Compliance (Safety Population)

| | Placebo | AVP-786-28 | AVP-786-42.63 |
|---|---|---|---|
| | (N = 210) | (N = 151) | (N = 160) |
| Compliance | 210 | 150 | 160 |
| Mean (SD) | 97.6 (6.3) | 97.7 (8.6) | 99.4 (12.9) |
| Median | 100.0 | 100.0 | 100.0 |
| Min, Max | 48, 105 | 31, 104 | 60, 200 |
| Compliance n (%) | 210 | 150 | 160 |
| <80% | 7 (3.3) | 3 (2.0) | 5 (3.1) |
| 80-120% | 203 (96.7) | 147 (98.0) | 153 (95.6) |
| >120% | 0 | 0 | 2 (1.3) |

SD = standard deviation
Note:
Denominators are the number of patients who had exposure data.

Efficacy Results and Tabulations of Individual Patient Data

Analysis of Efficacy

The following sections present the results of the analyses of the primary (CMAI Total score) and secondary efficacy endpoints. The impact of the FWE control procedure on the analyses and interpretation of the primary and key secondary efficacy endpoints are briefly addressed below.

Family-Wise Error Control

To control FWE, the difference between the average treatment effect of the average of AVP-786-28 and AVP-786-42.63 (combined effect) versus placebo was tested first, at significance level 2-sided α=0.05. This test was not significant (p=0.552). Since this test was not passed, the individual comparisons of each group versus placebo for the primary endpoint and the key secondary endpoints could not be evaluated under the FWE 2-sided α=0.05 level. Therefore, the results for the CMAI Total score, mADCS-CGIC-Agitation score, and CGIS-Agitation score are reported descriptively using nominal p-values for statistical significance.

Primary Efficacy Endpoint

Primary Analysis

The primary efficacy endpoint was the change from Baseline in the CMAI Total score at Week 12 for AVP-786-28 and AVP-786-42.63 versus placebo.

Patients treated with AVP-786-28 and AVP-786-42.63 showed declines (improvement) from Baseline in CMAI Total score at Week 12; however, the changes from Baseline were similar to those in the placebo group and were not significantly different from placebo (Table 66). The treatment differences (CI) versus placebo at Week 12 were 0.4 (−2.7 to 3.5; p=0.789) for AVP-786-28 and −2.0 (−5.0 to 1.0; p=0.200) for AVP-786-42.63.

For both active treatment groups combined (MMRM analysis), the difference in change from Baseline versus placebo was also not statistically significant (AVP-786-28 and AVP-786-42.63 p=0.552). The decrease from Baseline to Week 12 was greatest in the AVP-786-42.63 group, but it was not significantly different from placebo.

TABLE 66
CMAI Total Score: Change from Baseline MMRM (Observed Data) - mITT Population

| Parameter/Results | Placebo | | AVP-786-28 | | AVP-786-42.63 | |
|---|---|---|---|---|---|---|
| Baseline: N, Mean (SD) | 210, 73.7 | (21.13) | 150, 68.8 | (19.39) | 159, 71.3 | (20.87) |
| Week 6 Change from Baseline: N, Mean (SD) | 202, −11.2 | (15.79) | 137, −10.7 | (15.00) | 153, −11.8 | (14.52) |
| Change from Baseline: LS Mean (SE) ¹ | −12.3 | (1.45) | −14.2 | (1.62) | −14.1 | (1.64) |
| Treat Diff vs. Placebo: p-value (Dif, 95% CI) ¹ | | | 0.209 (−1.9, −4.9 to 1.1) | | 0.220 (−1.8, −4.8 to 1.1) | |
| Week 12 Change from Baseline: N, Mean (SD) | 188, −16.2 | (16.97) | 120, −12.7 | (16.21) | 141, −17.0 | (16.12) |
| Change from Baseline: LS Mean (SE) ¹ | −16.9 | (1.48) | −16.5 | (1.67) | −18.9 | (1.67) |

| | | | |
|---|---|---|---|
| Treat Diff vs. Placebo: p-value (Dif, 95% CI) ¹ | | 0.789 (0.4, −2.7 to 3.5) | 0.200 (−2.0, −5.0 to 1.0) |
| MMRM: N | 210 | 150 | 159 |

| | |
|---|---|
| Week 12 Change from Baseline: | Average AVP-786-28 and AVP-786-42.63 |
| Treat Diff vs. Placebo: p-value (Dif, 95% CI) ¹ | 0.552 (−0.8, −3.3 to 1.8) |

CI = confidence interval;
CMAI = Cohen-Mansfield Agitation Inventory;
Dif/Diff = difference;
LS = least squares;
MMRM = mixed model repeated measures;
NPI AA = Neuropsychiatric Inventory Agitation/Aggression domain;
SD = standard deviation;
SE = standard error
Note:
CMAI Total score ranges from 29 to 203 with higher scores indicating worsening condition.
¹ MMRMs include fixed effect treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no) and Cohort (Cohort 1 vs. Cohort 2). Unstructured variance-covariance was used.

The change from Baseline in the mean CMAI Total score at various time points is shown in the Table 66a below:

TABLE 66a
CMAI - Total Score: Change from Baseline MMRM - Observed Data (mITT Population)

| Parameter/Results | Placebo | | AVP-786 28 mg | | AVP-786 42.63 mg | |
|---|---|---|---|---|---|---|
| Baseline: N, Mean (SD) | 210, 73.7 | (21.13) | 150, 68.8 | (19.39) | 159, 71.3 | (20.87) |
| Week 1 Change from Baseline: N, Mean (SD) | 206, −5.1 | (11.41) | 142, −6.0 | (13.52) | 153, −6.0 | (11.92) |
| Change from Baseline: LS Mean (SE) ¹ | −6.6 | (1.33) | −8.7 | (1.45) | −8.1 | (1.50) |
| Treat Diff vs. Placebo: p-value (Dif, 95% CI) ¹ | | | 0.073 (−2.1, −4.5 to 0.2) | | 0.209 (−1.5, −3.9 to 0.8) | |
| Week 2 Change from Baseline: N, Mean (SD) | 185, −8.4 | (13.02) | 131, −9.1 | (13.22) | 149, −8.8 | (13.96) |
| Change from Baseline: LS Mean (SE) ¹ | −9.1 | (1.39) | −12.1 | (1.52) | −11.0 | (1.55) |
| Treat Diff vs. Placebo: p-value (Dif, 95% CI) ¹ | | | 0.026 (−3.0, −5.6 to −0.4) | | 0.153 (−1.9, −4.5 to 0.7) | |
| Week 3 Change from Baseline: N, Mean (SD) | 199, −9.8 | (16.06) | 146, −11.1 | (14.60) | 155, −10.3 | (13.09) |
| Change from Baseline: LS Mean (SE) ¹ | −11.0 | (1.41) | −14.2 | (1.55) | −12.6 | (1.59) |
| Treat Diff vs. Placebo: p-value (Dif, 95% CI) ¹ | | | 0.023 (−3.2, −5.9 to −0.4) | | 0.253 (−1.6, −4.3 to 1.1) | |
| Week 6 Change from Baseline: N, Mean (SD) | 202, −11.2 | (15.79) | 137, −10.7 | (15.00) | 153, −11.8 | (14,52) |
| Change from Baseline: LS Mean (SE) ¹ | −12.3 | (1.45) | −14.2 | (1.62) | −14.1 | (1.64) |
| Treat Diff vs. Placebo: p-value (Dif, 95% CI) ¹ | | | 0.209 (−1.9, −4.9 to 1.1) | | 0.220 (−1.8, −4.8 to 1.1) | |
| Week 9 Change from Baseline: N, Mean (SD) | 195, −15.2 | (16.78) | 120, −13.2 | (15.31) | 151, −15.0 | (15.86) |
| Change from Baseline: LS Mean (SE) ¹ | −16.0 | (1.45) | −17.0 | (1.63) | −17.3 | (1.64) |
| Treat Diff vs. Placebo: p-value (Dif, 95% CI) | | | 0.484 (−1.1, −4.0 to 1.9) | | 0.375 (−1.3, −4.2 to 1.6) | |
| Week 12 Change from Baseline: N, Mean (SD) | 188, −16.2 | (16.97) | 120, −12.7 | (16.21) | 141, −17.0 | (16.12) |
| Change from Baseline: LS Mean (SE) ¹ | −16.9 | (1.48) | −16.5 | (1.67) | −18.9 | (1.67) |

| | | | |
|---|---|---|---|
| Treat Diff vs. Placebo: p-value (Dif, 95% CI) ¹ | | 0.789 (0.4, −2.7 to 3.5) | 0.200 (−2.0, −5.0 to 1.0) |
| MMRM: N | 210 | 150 | 159 |

| | |
|---|---|
| Week 12 Change from Baseline: | Average AVP-786 28 and 42.63 mg |
| Treat Diff vs. Placebo: p-value (Dif, 95% CI) ¹ | 0.552 (−0.8, −3.3 to 1.8) |

Note:
CMAI Total Score ranges from 29 to 203 with higher scores indicating worsening condition.
¹ MMRMs include fixed effect treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no) and Cohort (Cohort 1 vs. Cohort 2). Unstructured variance-covariance was used.

The changes from Baseline in the mean CMAI Aggressive Behavior scores, CMAI Nonaggressive Behavior scores, and CMAI Verbal Agitation scores at various time points are shown in the tables below:

TABLE 66b
CMAI - Factor 1, Aggressive Behavior: Change from Baseline MMRM - Observed Data (mITT Population)

| Parameter/Results | Placebo | | AVP-786 28 mg | | AVP-786 42.63 mg | |
|---|---|---|---|---|---|---|
| Baseline: N, Mean (SD) | 210, 21.0 | (7.59) | 150, 19.4 | (7.81) | 159, 19.7 | (8.59) |
| Week 1 Change from Baseline: N, Mean (SD) | 206, −1.6 | (4.17) | 142, −2.2 | (5.36) | 153, −1.9 | (5.11) |
| Change from Baseline: LS Mean (SE) ¹ | −1.6 | (0.46) | −2.7 | (0.50) | −2.3 | (0.51) |
| Treat Diff vs. Placebo: p-value (Dif, 95% CI) ¹ | | | 0.009 (−1.1, −1.9 to −0.3) | | 0.103 (−0.7, −1.5 to 0.1) | |
| Week 2 Change from Baseline: N, Mean (SD) | 185, −2.6 | (5.02) | 131, −2.8 | (5.73) | 149, −2.7 | (6.00) |
| Change from Baseline: LS Mean (SE) ¹ | −2.5 | (0.48) | −3.5 | (0.52) | −3.1 | (0.53) |
| Treat Diff vs. Placebo: p-value (Dif, 95% CI) ¹ | | | 0.032 (−1.0, −1.9 to −0.1) | | 0.173 (−0.6, −1.5 to 0.3) | |
| Week 3 Change from Baseline: N, Mean (SD) | 199, −2.9 | (5.87) | 146, −3.4 | (6.22) | 155, −3.4 | (5.78) |
| Change from Baseline: LS Mean (SE) ¹ | −2.8 | (0.48) | −4.2 | (0.52) | −3.9 | (0.54) |
| Treat Diff vs. Placebo: p-value (Dif, 95% CI) ¹ | | | 0.003 (−1.4, −2.3 to −0.5) | | 0.027 (−1.0, −2.0 to −0.1) | |
| Week 6 Change from Baseline: N, Mean (SD) | 202, −3.3 | (6.48) | 137, −2.9 | (6.08) | 153, −3.5 | (5.83) |
| Change from Baseline: LS Mean (SE) ¹ | −3.1 | (0.51) | −3.9 | (0.57) | −4.0 | (0.57) |
| Treat Diff vs. Placebo: p-value (Dif, 95% CI) ¹ | | | 0.163 (−0.8, −1.8 to 0.3) | | 0.110 (−0.9, −1.9 to 0.2) | |
| Week 9 Change from Baseline: N, Mean (SD) | 195, −4.4 | (6.60) | 120, −3.6 | (5.83) | 151, −3.9 | (6.77) |
| Change from Baseline: LS Mean (SE) ¹ | −4.2 | (0.49) | −4.6 | (0.56) | −4.5 | (0.56) |
| Treat Diff vs. Placebo: p-value (Dif, 95% CI) ¹ | | | 0.422 (−0.4, −1.4 to 0.6) | | 0.500 (−0.3, −1.3 to 0.6) | |
| Week 12 Change from Baseline: N, Mean (SD) | 188, −4.6 | (6.64) | 120, −3.2 | (6.08) | 141, −4.5 | (6.95) |
| Change from Baseline: LS Mean (SE) ¹ | −4.3 | (0.50) | −4.2 | (0.56) | −5.1 | (0.56) |

| | | |
|---|---|---|
| Treat Diff vs. Placebo: p-value (Dif, 95% CI) ¹ | 0.787 (0.1, −0.9 to 1.2) | 0.133 (−0.8, −1.8 to 0.2) |

| | | | |
|---|---|---|---|
| MMRM: N | 210 | 150 | 159 |

| | |
|---|---|
| Week 12 Change from Baseline: | Average AVP-786 28 and 42.63 mg |
| Treat Diff vs. Placebo: p-value (Dif, 95% CI) ¹ | 0.470 (−0.3, −1.2 to 0.5) |

Note:
Factor 1, Aggressive Behavior ranges from 12 to 84 with higher scores indicating worsening condition.
¹ MMRMs include fixed effect treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no) and Cohort (Cohort 1 vs. Cohort 2). Unstructured variance-covariance was used.

TABLE 66c
CMAI - Factor 2, Physically Non-aggressive Behavior: Change
from Baseline MMRM - Observed Data (mITT Population)

| Parameter/Results | Placebo | | AVP-786 28 mg | | AVP-786 42.63 mg | |
|---|---|---|---|---|---|---|
| Baseline: N, Mean (SD) | 210, 21.0 | (7.99) | 150, 19.6 | (7.63) | 159, 20.5 | (7.50) |
| Week 1 Change from Baseline: N, Mean (SD) | 206, −1.5 | (3.96) | 142, −1.3 | (4.85) | 153, −1.5 | (3.79) |
| Change from Baseline: LS Mean (SE) ¹ | −2.2 | (0.48) | −2.2 | (0.52) | −2.3 | (0.54) |
| Treat Diff vs. Placebo: p-value (Dif, 95% CI) ¹ | | | 0.995 (−0.0, −0.8 to 0.8) | | 0.761 (−0.1, −1.0 to 0.7) | |
| Week 2 Change from Baseline: N, Mean (SD) | 185, −2.5 | (4.87) | 131, −2.6 | (4.84) | 149, −2.4 | (4.49) |
| Change from Baseline: LS Mean (SE) ¹ | −2.8 | (0.51) | −3.6 | (0.56) | −3.1 | (0.57) |
| Treat Diff vs. Placebo: p-value (Dif, 95% CI) ¹ | | | 0.101 (−0.8, −1.8 to 0.2) | | 0.547 (−0.3.−1.3 to 0.7) | |
| Week 3 Change from Baseline: N, Mean (SD) | 199, −2.7 | (5.71) | 146, −3.2 | (5.39) | 155, −2.9 | (4.93) |
| Change from Baseline: LS Mean (SE) ¹ | −3.3 | (0.52) | −4.1 | (0.57) | −3.7 | (0.58) |
| Treat Diff vs. Placebo: p-value (Dif, 95% CI) ¹ | | | 0.115 (−0.8, −1.8 to 0.2) | | 0.502 (−0.4, −1.4 to 0.7) | |
| Week 6 Change from Baseline: N, Mean (SD) | 202, −3.3 | (5.86) | 137, −3.4 | (5.56) | 153, −3.6 | (5.70) |
| Change from Baseline: LS Mean (SE) ¹ | −3.8 | (0.54) | −4.4 | (0.61) | −4.4 | (0.61) |
| Treat Diff vs. Placebo: p-value (Dif, 95% CI) ¹ | | | 0.305 (−0.6, −1.7 to 0.5) | | 0.275 (−0.6, −1.7 to 0.5) | |
| Week 9 Change from Baseline: N, Mean (SD) | 195, −4.3 | (5.88) | 120, −4.2 | (5.60) | 151, −4.8 | (5.48) |
| Change from Baseline: LS Mean (SE) ¹ | −4.7 | (0.53) | −5.1 | (0.59) | −5.6 | (0.59) |
| Treat Diff vs. Placebo: p-value (Dif, 95% CI) ¹ | | | 0.481 (−0.4, −1.5 to 0.7) | | 0.112 (−0.9, −1.9 to 0.2) | |
| Week 12 Change from Baseline: N, Mean (SD) | 188, −4.6 | (6.29) | 120, −3.8 | (6.14) | 141, −5.2 | (5.90) |
| Change from Baseline: LS Mean (SE) ¹ | −5.0 | (0.55) | −4.9 | (0.63) | −5.8 | (0.63) |

| | | |
|---|---|---|
| Treat Diff vs. Placebo: p-value (Dif, 95% CI) ¹ | 0.834 (0.1, −1.1 to 1.3) | 0.206 (−0.8, −1.9 to 0.4) |

| | | | |
|---|---|---|---|
| MMRM: N | 210 | 150 | 159 |

| | |
|---|---|
| Week 12 Change from Baseline: | Average AVP-786 28 and 42.63 mg |
| Treat Diff vs. Placebo: p-value (Dif, 95% CI) ¹ | 0.536 (−0.3, −1.3 to 0.7) |

Note:
Factor 2, Physically Non-aggressive Behavior ranges 6 to 42 with higher scores indicating worsening condition.
¹ MMRMs include fixed effect treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs no) and Cohort (Cohort 1 vs. Cohort 2). Unstructured variance-covariance was used.

TABLE 66d
CMAI - Factor 3, Verbally Agitated Behavior: Change from Baseline MMRM - Observed Data (mITT Population)

| Parameter/Results | Placebo | | AVP-786 28 mg | | AVP-786 42.63 mg | |
|---|---|---|---|---|---|---|
| Baseline: N, Mean (SD) | 210, 17.4 | (5.89) | 150, 16.2 | (5.16) | 158, 17.4 | (5.47) |
| Week 1 Change from Baseline: N, Mean (SD) | 206, −1.1 | (3.85) | 142, −1.4 | (3.63) | 152, −1.4 | (3.41) |
| Change from Baseline: LS Mean (SE) ¹ | −1.6 | (0.39) | −2.3 | (0.43) | −1.8 | (0.44) |
| Treat Diff vs. Placebo: p-value (Dif, 95% CI) ¹ | | | 0.072 (−0.7, −1.4 to 0.1) | | 0.585 (−0.2, −0.9 to 0.5) | |
| Week 2 Change from Baseline: N, Mean (SD) | 185, −2.1 | (3.98) | 131, −2.0 | (4.16) | 148, −2.2 | (4.18) |
| Change from Baseline: LS Mean (SE) ¹ | −2.4 | (0.41) | −2.8 | (0.46) | −2.7 | (0.46) |
| Treat Diff vs. Placebo: p-value (Dif, 95% CI) ¹ | | | 0.258 (−0.5, −1.3 to 0.3) | | 0.371 (−0.4. −1.2 to 0.4) | |
| Week 3 Change from Baseline: N, Mean (SD) | 199, −2.6 | (4.74) | 146, −2.7 | (4.46) | 154, −2.5 | (4.37) |
| Change from Baseline: LS Mean (SE) ¹ | −3.0 | (0.43) | −3.6 | (0.47) | −2.9 | (0.48) |
| Treat Diff vs. Placebo: p-value (Dif, 95% CI) ¹ | | | 0.151 (−0.6, −1.5 to 0.2) | | 0.913 (0.0, −0.8 to 0.9) | |
| Week 6 Change from Baseline: N, Mean (SD) | 202, −2.8 | (4.33) | 137, −2.6 | (4.48) | 152, −2.9 | (4.54) |
| Change from Baseline: LS Mean (SE) ¹ | −3.3 | (0.43) | −3.6 | (0.48) | −3.4 | (0.48) |
| Treat Diff vs. Placebo: p-value (Dif, 95% CI) ¹ | | | 0.472 (−0.3, −1.2 to 0.6) | | 0.792 (−0.1, −1.0 to 0.8) | |
| Week 9 Change from Baseline: N, Mean (SD) | 195, −3.9 | (4.72) | 120, −3.0 | (4.58) | 150, −4.1 | (4.90) |
| Change from Baseline: LS Mean (SE) ¹ | −4.3 | (0.43) | −4.1 | (0.49) | −4.5 | (0.49) |
| Treat Diff vs. Placebo: p-value (Dif, 95% CI) ¹ | | | 0.660 (0.2, −0.7 to 1.1) | | 0.788 (−0.1, −1.0 to 0.8) | |
| Week 12 Change from Baseline: N, Mean (SD) | 188, −4.3 | (4.47) | 120, −3.4 | (4.88) | 140, −4.9 | (4.74) |
| Change from Baseline: LS Mean (SE) ¹ | −4.7 | (0.44) | −4.4 | (0.50) | −5.2 | (0.50) |

| | | | |
|---|---|---|---|
| Treat Diff vs. Placebo: p-value (Dif, 95% CI) ¹ | | 0.484 (0.3, −0.6 to 1.3) | 0.360 (−0.4, −1.4 to 0.5) |
| MMRM: N | 210 | 150 | 158 |

| | |
|---|---|
| Week 12 Change from Baseline: | Average AVP-786 28 and 42.63 mg |
| Treat Diff vs. Placebo: p-value (Dif, 95% CI) ¹ | 0.907 (−0.0, −0.8 to 0.7) |

Note:
Factor 3, Verbally Agitated Behavior ranges 4 to 28 with higher scores indicating worsening condition.
¹ MMRMs include fixed effect treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no) and Cohort (Cohort 1 vs. Cohort 2). Unstructured variance-covariance was used.

### 4.3.1.1.1. Sensitivity Analyses

The sensitivity analysis on the primary efficacy endpoint using various statistical analyses methods corroborated the findings of the primary analysis and is summarized in Table 67. The results were similar to the primary analysis; neither active treatment group was significantly different from placebo with the MMRM using observed data in the ITT Population.

TABLE 67
Summary of the Primary Analysis and Sensitivity Analyses of the CMAI Total Score at Week 12

| | Placebo | | AVP-786-28 | | AVP-786-42.63 | |
|---|---|---|---|---|---|---|
| Primary Analysis | | | | | | |
| Week 12 Change from Baseline: | 188, −16.2 | (16.97) | 120, −12.7 | (16.21) | 141, −17.0 | (16.12) |
| N, Mean (SD) | | | | | | |
| Change from Baseline: LS Mean (SE) ¹ | −16.9 | (1.48) | −16.5 | (1.67) | −18.9 | (1.67) |
| Treat Diff vs. Placebo: p-value (Dif, 95% | | | 0.789 (0.4, −2.7 to 3.5) | | 0.200 (−2.0, −5.0 to 1.0) | |
| CI) ¹ | | | | | | |
| MMRM: N | 210 | | 150 | | 159 | |
| Week 12 Change from Baseline: | | | 0.552 (−0.8, −3.3 to 1.8) | | | |
| Treat Diff vs. Placebo: p-value (Dif, 95% CI) ¹ | | | (average AVP-786-28 and 42.63) | | | |
| Sensitivity Analyses | | | | | | |
| MMRM - Observed Data (ITT) | | | | | | |
| Week 12 Change from Baseline: | 188, −16.2 | (16.97) | 120, −12.7 | (16.21) | 141, −17.0 | (16.12) |
| N, Mean (SD) | | | | | | |
| Change from Baseline: LS Mean (SE) ¹ | −16.9 | (1.48) | −16.5 | (1.67) | −18.9 | (1.67) |
| Treat Diff vs. Placebo: p-value (Dif, 95% | | | 0.789 (0.4, −2.7 to 3.5) | | 0.200 (−2.0, −5.0 to 1.0) | |
| CI) ¹ | | | | | | |
| MMRM: N | 210 | | 150 | | 159 | |
| Week 12 Change from Baseline: | | | 0.552 (−0.8, −3.3 to 1.8) | | | |
| Treat Diff vs. Placebo: p-value (Dif, 95% CI) ¹ | | | (average AVP-786-28 and 42.63) | | | |
| Observed Data Excluding Patients with | | | | | | |
| Consent Errors ² | | | | | | |
| Week 12 Change from Baseline: | 185, −16.4 | (17.02) | 117, −12.6 | (16.36) | 140, −17.0 | (16.17) |
| N, Mean (SD) | | | | | | |
| Change from Baseline: LS Mean (SE) ¹ | −16.9 | (1.48) | −16.5 | (1.69) | −18.7 | (1.68) |

| | | |
|---|---|---|
| Treat Diff vs. Placebo: p-value (Dif, 95% | 0.808 (0.4, −2.8 to 3.5) | 0.248 (−1.8, −4.8 to 1.3) |
| CI) ¹ | | |

| | | | |
|---|---|---|---|
| MMRM: N | 207 | 147 | 158 |

| | |
|---|---|
| Week 12 Change from Baseline: | 0.595 (−0.7, −3.3 to 1.9) |
| Treat Diff vs. Placebo: p-value (Dif, 95% CI) ¹ | (average AVP-786-28 and 42.63) |

CI = confidence interval;
CMAI = Cohen-Mansfield Agitation Inventory;
Dif/Diff = difference;
ITT = intent-to-treat;
LS = least squares;
mITT = modified intent-to-treat;
MMRM = mixed model repeated measures;
SD = standard deviation;
SE = standard error
CMAI Total score ranges from 29 to 203 with higher scores indicating worsening condition
¹ MMRMs include fixed effect treatment, visit, treatment-by-visit, Baseline, Baseline-by-visit, Baseline NPI - Agitation/Aggression (≤6 vs >6), risk assessment for falls (normal/mild vs moderate/severe), Baseline concomitant use of antipsychotic medications (yes vs no) and Cohort (Cohort 1 vs Cohort 2). Unstructured variance-covariance was used.

### 4.3.1.2. CMAI Subscales

The CMAI subscales F1—Aggressive Behavior, F2—Physically Nonaggressive Behavior, and F3—Verbally Agitated Behavior are summarized in Table 68. For both active treatment groups, the treatment difference in change from Baseline to Week 12 in CMAI F1—Aggressive Behavior, CMAI F2—Physically Nonaggressive Behavior, or CMAI F3—Verbally Agitated Behavior was not statistically significant versus placebo. Mean changes from baseline in these subscale scores are provided in data tables herein.

The results of the sensitivity analyses of the CMAI subscales were similar to those for the primary analysis, with no significant between-group differences in the overall analysis (for the ITT MMRM) or at Week 12.

TABLE 68
CMAI Subscale Scores at Week 12: Change from Baseline MMRM (Observed Data) - mITT Population

| Parameter/Results | Placebo | | AVP-786-28 | | AVP-786-42.63 | |
|---|---|---|---|---|---|---|
| CMAI: F1-Aggressive Behavior | | | | | | |
| Baseline: N, Mean (SD) | 210, 21.0 | (7.59) | 150, 19.4 | (7.81) | 159, 19.7 | (8.59) |
| Week 12 Change from Baseline: N, Mean (SD) | 188, −4.6 | (6.64) | 120, −3.2 | (6.08) | 141, −4.5 | (6.95) |
| Change from Baseline: LS Mean (SE) ¹ | −4.3 | (0.50) | −4.2 | (0.56) | −5.1 | (0.56) |
| Treat Diff vs. Placebo: p-value (Dif, 95% CI) ¹ | | | 0.787 (0.1, −0.9 to 1.2) | | 0.133 (−0.8, −1.8 to 0.2) | |
| MMRM: N | 210 | | 150 | | 159 | |
| Treat Diff vs. Placebo: p-value (Dif, 95% CI) ¹ | | | 0.470 (−0.3, −1.2 to 0.5) | | | |
| | | | (average AVP-786-28 and 42.63) | | | |
| CMAI: F2-Physically Nonaggressive Behavior | | | | | | |
| Baseline: N, Mean (SD) | 210, 21.0 | (7.99) | 150, 19.6 | (7.63) | 159, 20.5 | (7.50) |
| Week 12 Change from Baseline: N, Mean (SD) | 188, −4.6 | (6.29) | 120, −3.8 | (6.14) | 141, −5.2 | (5.90) |
| Change from Baseline: LS Mean (SE) ¹ | −5,0 | (0.55) | −4.9 | (0.63) | −5.8 | (0.63) |
| Treat Diff vs. Placebo: p-value (Dif, 95% CI) ¹ | | | 0.834 (0.1, −1.1 to 1.3) | | 0.206 (−0,8, −1.9 to 0.4) | |
| MMRM: N | 210 | | 150 | | 159 | |
| Treat Diff vs. Placebo: p-value (Dif, 95% CI) ¹ | | | 0.536 (−0.3, −1.3 to 0.7) | | | |
| | | | (average AVP-786-28 and 42.63) | | | |
| CMAI: F3-Verbally Agitated Behavior | | | | | | |
| Baseline: N, Mean (SD) | 210, 17.4 | (5.89) | 150,16.2 | (5.16) | 158, 17.4 | (5.47) |
| Week 12 Change from Baseline: N, Mean (SD) | 188, −4.3 | (4.47) | 120, −3.4 | (4.88) | 1.40, −4.9 | (4.74) |
| Change from Baseline: LS Mean (SE) ¹ | −4.7 | (0,44) | −4.4 | (0.50) | −5.2 | (0.50) |

| | | | |
|---|---|---|---|
| Treat Diff vs. Placebo: p-value (Dif, 95% CI) ¹ | | 0.484 (0.3, −0.6 to 1.3) | 0.360 (−0.4, −1.4 to 0.5) |
| MMRM: N | 210 | 150 | 158 |

| | |
|---|---|
| Treat Diff vs. Placebo: p-value (Dif, 95% CI) ¹ | 0.907 (−0.0, −0.8 to 0.7) |
| | (average AVP-786-28 and 42.63) |

CI = confidence interval;
CMAI = Cohen-Mansfield Agitation Inventory;
Dif/Diff = difference;
ITT = intent-to-treat;
LS = least squares;
mITT = modified intent-to-treat;
MMRM = mixed model repeated measures;
NPI AA = Neuropsychiatric Inventory Agitation/Aggression domain;
SD = standard deviation;
SE = standard error
Note:
Factor 1, F1-Aggressive Behavior ranges from 12 to 84. Factor 2, F2-Physically Nonaggressive Behavior ranges 6 to 42. Factor 3, F3-Verbally Agitated Behavior ranges 4 to 28. For all factors, higher scores indicating worsening condition.
¹ MMRMs include fixed effect treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no) and Cohort (Cohort 1 vs. Cohort 2). Unstructured variance-covariance was used.

CMAI Total Score—Change from Baseline in Patient Sub-Groups

Tables 68A-68Z below show changes from baseline in CMAI Total score in the patient subgroups identified in each Table:

TABLE 68A
SUMMARY OF MEAN CHANGE FROM BASELINE TO WEEK 12 IN CMAI AGGRESSIVE
BEHAVIOR - MMRM, UN (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 99 | 22.7 | 7.75 | | | | | | | | |
| | AVP-786 42.63 mg | 104 | 23.3 | 8.64 | | | | | | | | |
| | PLACEBO | 166 | 23.1 | 7.21 | | | | | | | | |
| WEEK 1 | AVP-786 28 mg | 95 | 19.4 | 6.32 | −3.33 | 0.46 | −3.3 | 6.24 | −1.16 | −2.29 | −0.04 | 0.0423 |
| | AVP-786 42.63 mg | 99 | 20.3 | 6.56 | −2.87 | 0.52 | −3.0 | 5.93 | −0.70 | −1.84 | 0.43 | 0.2233 |
| | PLACEBO | 162 | 20.8 | 6.57 | −2.17 | 0.38 | −2.2 | 4.44 | | | | |
| WEEK 2 | AVP-786 28 mg | 84 | 18.6 | 6.27 | −4.32 | 0.51 | −4.2 | 6.67 | −1.02 | −2.27 | 0.24 | 0.1112 |
| | AVP-786 42.63 mg | 97 | 19.3 | 6.33 | −3.92 | 0.56 | −4.2 | 6.90 | −0.62 | −1.86 | 0.62 | 0.3252 |
| | PLACEBO | 147 | 19.7 | 6.40 | −3.30 | 0.42 | −3.4 | 5.32 | | | | |
| WEEK 3 | AVP-786 28 mg | 95 | 17.7 | 6.17 | −5.28 | 0.52 | −5.1 | 7.12 | −1.61 | −2.90 | −0.33 | 0.0141 |
| | AVP-786 42.63 mg | 101 | 18.0 | 6.16 | −4.96 | 0.57 | −5.2 | 6.35 | −1.30 | −2.58 | −0.01 | 0.0483 |
| | PLACEBO | 156 | 19.3 | 6.24 | −3.66 | 0.43 | −3.8 | 6.33 | | | | |
| WEEK 6 | AVP-786 28 mg | 90 | 17.9 | 6.41 | −4.98 | 0.61 | −4.5 | 6.88 | −0.89 | −2.38 | 0.59 | 0.2363 |
| | AVP-786 42.63 mg | 99 | 18.0 | 7.42 | −4.99 | 0.64 | −5.2 | 6.57 | −0.90 | −2.37 | 0.56 | 0.2258 |
| | PLACEBO | 158 | 19.0 | 6.81 | −4.09 | 0.48 | −4.2 | 6.98 | | | | |
| WEEK 9 | AVP-786 28 mg | 77 | 16.8 | 5.55 | −5.82 | 0.59 | −5.4 | 6.50 | −0.29 | −1.71 | 1.13 | 0.6872 |
| | AVP-786 42.63 mg | 98 | 17.2 | 6.62 | −5.78 | 0.61 | −6.0 | 7.57 | −0.25 | −1.63 | 1.12 | 0.7189 |
| | PLACEBO | 154 | 17.5 | 6.03 | −5.53 | 0.45 | −5.7 | 6.87 | | | | |
| WEEK 12 | AVP-786 28 mg | 78 | 17.9 | 6.58 | −5.11 | 0.59 | −4.8 | 7.05 | 0.46 | −0.96 | 1.89 | 0.5233 |
| | AVP-786 42.63 mg | 92 | 16.3 | 5.33 | −6.61 | 0.61 | −6.8 | 7.63 | −1.04 | −2.43 | 0.34 | 0.1392 |
| | PLACEBO | 147 | 17.5 | 6.08 | −5.57 | 0.46 | −5.8 | 7.01 | | | | |
| | AVERAGE | | | | | | | | −0.29 | −1.45 | 0.86 | 0.6209 |

TABLE 68B
SUMMARY OF MEAN CHANGE FROM BASELINE TO WEEK 12 IN CMAI PHYSICALLY NON-AGGRESSIVE
BEHAVIOR - MMRM, UN (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 99 | 20.7 | 7.61 | | | | | | | | |
| | AVP-786 42.63 mg | 104 | 21.4 | 7.19 | | | | | | | | |
| | PLACEBO | 166 | 21.7 | 8.03 | | | | | | | | |
| WEEK 1 | AVP-786 28 mg | 95 | 18.8 | 7.32 | −2.11 | 0.40 | −1.9 | 4.96 | −0.55 | −1.53 | 0.43 | 0.2679 |
| | AVP-786 42.63 mg | 99 | 19.6 | 6.96 | −2.05 | 0.46 | −1.8 | 3.35 | −0.49 | −1.48 | 0.50 | 0.3331 |
| | PLACEBO | 162 | 20.3 | 7.80 | −1.56 | 0.33 | −1.4 | 4.04 | | | | |
| WEEK 2 | AVP-786 28 mg | 84 | 18.3 | 7.36 | −3.35 | 0.48 | −2.9 | 4.44 | −0.95 | −2.11 | 0.20 | 0.1055 |
| | AVP-786 42.63 mg | 97 | 18.9 | 6.35 | −2.93 | 0.51 | −2.7 | 4.32 | −0.53 | −1.67 | 0.61 | 0.3574 |
| | PLACEBO | 147 | 19.6 | 7.69 | −2.39 | 0.38 | −2.5 | 4.85 | | | | |
| WEEK 3 | AVP-786 28 mg | 95 | 17.4 | 7.26 | −3.84 | 0.51 | −3.6 | 4.85 | −0.99 | −2.24 | 0.25 | 0.1158 |
| | AVP-786 42.63 mg | 101 | 18.3 | 7.32 | −3.32 | 0.55 | −3.1 | 4.96 | −0.48 | −1.72 | 0.76 | 0.4489 |
| | PLACEBO | 156 | 19.0 | 7.45 | −2.84 | 0.41 | −2.8 | 5.80 | | | | |
| WEEK 6 | AVP-786 28 mg | 90 | 17.1 | 7.77 | −3.95 | 0.58 | −3.6 | 5.47 | −0.71 | −2.12 | 0.71 | 0.3250 |
| | AVP-786 42.63 mg | 99 | 17.2 | 7.23 | −4.46 | 0.60 | −4.1 | 6.05 | −1.22 | −2.61 | 0.17 | 0.0859 |
| | PLACEBO | 158 | 18.6 | 8.07 | −3.24 | 0.45 | −3.2 | 6.04 | | | | |
| WEEK 9 | AVP-786 28 mg | 77 | 16.6 | 7.46 | −4.68 | 0.55 | −4.5 | 5.73 | −0.29 | −1.63 | 1.04 | 0.6685 |
| | AVP-786 42.63 mg | 98 | 16.1 | 6.37 | −5.54 | 0.57 | −5.3 | 5.72 | −1.15 | −2.45 | 0.15 | 0.0833 |
| | PLACEBO | 154 | 17.5 | 7.26 | −4.39 | 0.43 | −4.4 | 6.04 | | | | |
| WEEK 12 | AVP-786 28 mg | 78 | 17.3 | 8.00 | −4.23 | 0.61 | −3.7 | 6.29 | 0.58 | −0.90 | 2.05 | 0.4439 |
| | AVP-786 42.63 mg | 92 | 15.6 | 6.51 | −6.04 | 0.62 | −5.9 | 6.00 | −1.23 | −2.67 | 0.20 | 0.0922 |
| | PLACEBO | 147 | 17.3 | 7.44 | −4.81 | 0.47 | −4.9 | 6.42 | | | | |
| | AVERAGE | | | | | | | | −0.33 | −1.53 | 0.87 | 0.5917 |

TABLE 68C
SUMMARY OF MEAN CHANGE FROM BASELINE TO WEEK 12 IN CMAI VERBALLY AGITATED
BEHAVIOR - MMRM, UN (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 99 | 16.9 | 5.37 | | | | | | | | |
| | AVP-786 42.63 mg | 103 | 18.5 | 5.43 | | | | | | | | |
| | PLACEBO | 166 | 18.1 | 5.75 | | | | | | | | |
| WEEK 1 | AVP-786 28 mg | 95 | 15.1 | 5.29 | −2.11 | 0.36 | −1.9 | 3.65 | −0.99 | −1.87 | −0.12 | 0.0254 |
| | AVP-786 42.63 mg | 98 | 17.1 | 5.15 | −1.26 | 0.40 | −1.5 | 3.57 | −0.14 | −1.02 | 0.73 | 0.7471 |
| | PLACEBO | 162 | 16.9 | 5.74 | −1.12 | 0.29 | −1.1 | 3.79 | | | | |
| WEEK 2 | AVP-786 28 mg | 84 | 15.4 | 5.52 | −2.34 | 0.41 | −2.1 | 4.01 | −0.51 | −1.49 | 0.48 | 0.3126 |
| | AVP-786 42.63 mg | 96 | 15.9 | 5.31 | −2.42 | 0.43 | −2.6 | 4.23 | −0.58 | −1.56 | 0.39 | 0.2393 |
| | PLACEBO | 147 | 16.2 | 5.48 | −1.83 | 0.32 | −2.0 | 3.98 | | | | |
| WEEK 3 | AVP-786 28 mg | 95 | 14.2 | 5.79 | −3.23 | 0.42 | −2.9 | 4.19 | −0.79 | −1.83 | 0.25 | 0.1352 |
| | AVP-786 42.63 mg | 100 | 15.5 | 5.37 | −2.67 | 0.46 | −2.9 | 3.94 | −0.24 | −1.27 | 0.80 | 0.6565 |
| | PLACEBO | 156 | 15.6 | 5.73 | −2.44 | 0.34 | −2.5 | 4.87 | | | | |
| WEEK 6 | AVP-786 28 mg | 90 | 14.1 | 6.13 | −3.12 | 0.44 | −2.8 | 4.47 | −0.31 | −1.39 | 0.76 | 0.5686 |
| | AVP-786 42.63 mg | 98 | 14.9 | 5.57 | −3.32 | 0.47 | −3.5 | 4.37 | −0.51 | −1.57 | 0.55 | 0.3474 |
| | PLACEBO | 158 | 15.2 | 5.60 | −2.81 | 0.35 | −2.9 | 4.41 | | | | |
| WEEK 9 | AVP-786 28 mg | 77 | 13.7 | 5.80 | −4.05 | 0.48 | −3.5 | 4.71 | 0.00 | −1.14 | 1.15 | 0.9975 |
| | AVP-786 42.63 mg | 97 | 13.9 | 5.28 | −4.38 | 0.48 | −4.7 | 4.55 | −0.33 | −1.44 | 0.77 | 0.5513 |
| | PLACEBO | 154 | 14.0 | 5.49 | −4.05 | 0.36 | −4.2 | 4.92 | | | | |
| WEEK 12 | AVP-786 28 mg | 78 | 14.1 | 6.13 | −3.96 | 0.49 | −3.6 | 5.02 | 0.40 | −0.79 | 1.58 | 0.5081 |
| | AVP-786 42.63 mg | 91 | 13.4 | 5.64 | −4.92 | 0.50 | −5.2 | 4.60 | −0.55 | −1.70 | 0.60 | 0.3448 |
| | PLACEBO | 147 | 13.7 | 5.65 | −4.36 | 0.37 | −4.5 | 4.64 | | | | |
| | AVERAGE | | | | | | | | −0.08 | −1.04 | 0.88 | 0.8746 |

TABLE 68D
SUMMARY OF MEAN CHANGE FROM BASELINE TO WEEK 12 IN CMAI TOTAL SCORE - MMRM,
UN IN AGE >=65 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 89 | 74.7 | 19.76 | | | | | | | | |
| | AVP-786 42.63 mg | 94 | 78.0 | 21.44 | | | | | | | | |
| | PLACEBO | 153 | 77.9 | 21.63 | | | | | | | | |
| WEEK 1 | AVP-786 28 mg | 85 | 66.2 | 18.82 | −9.28 | 1.30 | −8.5 | 15.84 | −3.25 | −6.44 | −0.07 | 0.0453 |
| | AVP-786 42.63 mg | 89 | 70.8 | 19.44 | −7.90 | 1.49 | −7.5 | 12.19 | −1.87 | −5.05 | 1.32 | 0.2499 |
| | PLACEBO | 149 | 72.0 | 20.65 | −6.03 | 1.07 | −5.7 | 12.01 | | | | |
| WEEK 2 | AVP-786 28 mg | 74 | 65.4 | 19.34 | −11.7 | 1.43 | −10.6 | 13.42 | −2.90 | −6.40 | 0.59 | 0.1033 |
| | AVP-786 42.63 mg | 87 | 67.2 | 18.87 | −11.7 | 1.57 | −11.4 | 14.66 | −2.83 | −6.27 | 0.60 | 0.1056 |
| | PLACEBO | 135 | 69.7 | 20.42 | −8.83 | 1.16 | −9.2 | 13.76 | | | | |
| WEEK 3 | AVP-786 28 mg | 85 | 61.8 | 19.27 | −14.4 | 1.51 | −13.3 | 14.74 | −3.66 | −7.38 | 0.07 | 0.0542 |
| | AVP-786 42.63 mg | 91 | 64.7 | 19.16 | −13.2 | 1.66 | −12.9 | 13.04 | −2.52 | −6.21 | 1.18 | 0.1814 |
| | PLACEBO | 143 | 67.4 | 20.14 | −10.7 | 1.23 | −10.6 | 17.55 | | | | |
| WEEK 6 | AVP-786 28 mg | 82 | 61.3 | 19.79 | −14.3 | 1.66 | −13.0 | 16.00 | −2.06 | −6.14 | 2.02 | 0.3215 |
| | AVP-786 42.63 mg | 89 | 62.8 | 20.90 | −15.3 | 1.78 | −14.8 | 15.53 | −3.05 | −7.07 | 0.96 | 0.1359 |
| | PLACEBO | 145 | 66.1 | 21.18 | −12.3 | 1.32 | −12.3 | 17.04 | | | | |
| WEEK 9 | AVP-786 28 mg | 69 | 58.9 | 19.63 | −17.5 | 1.70 | −16.2 | 16.98 | −0.64 | −4.77 | 3.48 | 0.7590 |
| | AVP-786 42.63 mg | 89 | 59.4 | 18.95 | −18.9 | 1.76 | −18.4 | 17.06 | −2.03 | −6.02 | 1.96 | 0.3178 |
| | PLACEBO | 141 | 61.5 | 19.33 | −16.9 | 1.32 | −17.1 | 17.68 | | | | |
| WEEK 12 | AVP-786 28 mg | 71 | 61.9 | 21.54 | −16.0 | 1.76 | −14.0 | 18.70 | 1.71 | −2.56 | 5.99 | 0.4316 |
| | AVP-786 42.63 mg | 83 | 57.0 | 18.23 | −21.3 | 1.82 | −21.0 | 16.34 | −3.64 | −7.78 | 0.51 | 0.0854 |
| | PLACEBO | 134 | 60.9 | 19.13 | −17.7 | 1.36 | −18.1 | 18.03 | | | | |

TABLE 68E
SUMMARY OF MEAN CHANGE FROM BASELINE TO WEEK 12 IN CMAI TOTAL SCORE - MMRM,
UN IN BASELINE CMAI <50 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 4 | 44.8 | 5.85 | | | | | | | | |
| | AVP-786 42.63 mg | 5 | 45.4 | 3.65 | | | | | | | | |
| | PLACEBO | 8 | 46.3 | 2.76 | | | | | | | | |
| WEEK 1 | AVP-786 28 mg | 4 | 43.0 | 6.16 | −5.50 | 4.94 | −1.8 | 0.96 | −4.76 | −16.6 | 7.06 | 0.3943 |
| | AVP-786 42.63 mg | 5 | 44.0 | 7.42 | −5.21 | 4.64 | −1.4 | 7.20 | −4.47 | −15.6 | 6.63 | 0.3954 |
| | PLACEBO | 8 | 46.5 | 8.59 | −0.75 | 2.94 | 0.3 | 8.22 | | | | |
| WEEK 2 | AVP-786 28 mg | 3 | 44.3 | 7.23 | −5.42 | 4.78 | 0.3 | 0.58 | −7.21 | −18.0 | 3.57 | 0.1719 |
| | AVP-786 42.63 mg | 5 | 40.0 | 3.39 | −9.45 | 4.19 | −5.4 | 4.34 | −11.2 | −20.7 | −1.75 | 0.0240 |
| | PLACEBO | 8 | 48.9 | 8.53 | 1.79 | 2.49 | 2.6 | 9.24 | | | | |
| WEEK 3 | AVP-786 28 mg | 4 | 41.0 | 5.42 | −8.04 | 4.92 | −3.8 | 5.50 | −7.45 | −19.2 | 4.33 | 0.1908 |
| | AVP-786 42.63 mg | 5 | 41.0 | 5.05 | −8.45 | 4.62 | −4.4 | 6.11 | −7.86 | −18.9 | 3.21 | 0.1463 |
| | PLACEBO | 8 | 46.5 | 8.57 | −0.59 | 2.93 | 0.3 | 8.38 | | | | |
| WEEK 6 | AVP-786 28 mg | 3 | 43.0 | 10.44 | −6.06 | 6.20 | −4.7 | 9.87 | −8.26 | −22.4 | 5.84 | 0.2311 |
| | AVP-786 42.63 mg | 5 | 38.6 | 5.32 | −11.3 | 5.05 | −6.8 | 5.72 | −13.5 | −25.7 | −1.25 | 0.0332 |
| | PLACEBO | 8 | 49.0 | 11.03 | 2.19 | 3.36 | 2.8 | 12.31 | | | | |
| WEEK 9 | AVP-786 28 mg | 3 | 41.7 | 10.79 | −8.15 | 5.45 | −6.0 | 10.44 | −5.03 | −17.7 | 7.60 | 0.4075 |
| | AVP-786 42.63 mg | 5 | 39.4 | 5.81 | −10.2 | 4.84 | −6.0 | 6.40 | −7.06 | −18.6 | 4.48 | 0.2098 |
| | PLACEBO | 8 | 43.9 | 8.82 | −3.12 | 3.15 | −2.4 | 9.26 | | | | |
| WEEK 12 | AVP-786 28 mg | 2 | 36.0 | 0.00 | −10.0 | 5.96 | −11.5 | 0.71 | −9.00 | −23.3 | 5.26 | 0.1959 |
| | AVP-786 42.63 mg | 5 | 37.0 | 6.28 | −12.3 | 5.24 | −8.4 | 8.08 | −11.3 | −24.2 | 1.65 | 0.0817 |
| | PLACEBO | 8 | 46.1 | 10.66 | −1.04 | 3.52 | −0.1 | 9.78 | | | | |

TABLE 68F
SUMMARY OF MEAN CHANGE FROM BASELINE TO WEEK 12 IN CMAI TOTAL SCORE - MMRM,
UN IN BASELINE CMAI <65 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 33 | 56.5 | 6.03 | | | | | | | | |
| | AVP-786 42.63 mg | 31 | 56.4 | 6.01 | | | | | | | | |
| | PLACEBO | 49 | 56.3 | 6.03 | | | | | | | | |
| WEEK 1 | AVP-786 28 mg | 33 | 51.7 | 10.71 | −5.00 | 1.31 | −4.7 | 9.48 | −2.36 | −5.68 | 0.96 | 0.1614 |
| | AVP-786 42.63 mg | 30 | 53.1 | 7.27 | −4.11 | 1.58 | −3.2 | 6.23 | −1.47 | −5.04 | 2.09 | 0.4150 |
| | PLACEBO | 48 | 54.1 | 8.43 | −2.64 | 1.10 | −2.3 | 6.56 | | | | |
| WEEK 2 | AVP-786 28 mg | 28 | 49.4 | 9.40 | −7.95 | 1.59 | −7.4 | 9.44 | −4.80 | −8.83 | −0.77 | 0.0200 |
| | AVP-786 42.63 mg | 28 | 51.8 | 10.16 | −5.70 | 1.80 | −4.6 | 8.19 | −2.54 | −6.71 | 1.63 | 0.2289 |
| | PLACEBO | 42 | 53.2 | 8.44 | −3.15 | 1.33 | −2.9 | 8.94 | | | | |
| WEEK 3 | AVP-786 28 mg | 31 | 47.8 | 11.98 | −9.37 | 1.85 | −8.6 | 11.84 | −6.96 | −11.7 | −2.26 | 0.0041 |
| | AVP-786 42.63 mg | 31 | 49.6 | 9.73 | −7.79 | 2.04 | −6.8 | 9.11 | −5.38 | −10.2 | −0.51 | 0.0308 |
| | PLACEBO | 46 | 54.2 | 11.41 | −2.41 | 1.53 | −2.2 | 10.16 | | | | |
| WEEK 6 | AVP-786 28 mg | 29 | 51.6 | 16.99 | −5.70 | 2.07 | −5.1 | 16.14 | −3.52 | −8.74 | 1.70 | 0.1837 |
| | AVP-786 42.63 mg | 31 | 46.3 | 7.54 | −11.1 | 2.20 | −10.1 | 7.84 | −8.90 | −14.2 | −3.60 | 0.0012 |
| | PLACEBO | 46 | 54.6 | 10.35 | −2.18 | 1.67 | −1.9 | 9.58 | | | | |
| WEEK 9 | AVP-786 28 mg | 24 | 48.3 | 14.44 | −9.87 | 2.01 | −8.4 | 14.11 | −4.13 | −9.15 | 0.89 | 0.1062 |
| | AVP-786 42.63 mg | 30 | 46.0 | 7.44 | −11.2 | 2.08 | −10.3 | 6.65 | −5.42 | −10.4 | −0.43 | 0.0334 |
| | PLACEBO | 44 | 51.3 | 11.42 | −5.74 | 1.58 | −5.0 | 10.04 | | | | |
| WEEK 12 | AVP-786 28 mg | 24 | 50.5 | 19.13 | −7.42 | 2.53 | −6.8 | 18.60 | −1.13 | −7.51 | 5.25 | 0.7259 |
| | AVP-786 42.63 mg | 29 | 44.9 | 8.95 | −12.6 | 2.55 | −11.4 | 8.60 | −6.29 | −12.6 | 0.01 | 0.0502 |
| | PLACEBO | 40 | 50.6 | 12.63 | −6.29 | 2.02 | −5.7 | 11.83 | | | | |

TABLE 68G
SUMMARY OF MEAN CHANGE FROM BASELINE TO WEEK 12 IN CMAI TOTAL SCORE - MMRM,
UN IN BASLINE NPI-AA >6 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 36 | 79.6 | 23.16 | | | | | | | | |
| | AVP-786 42.63 mg | 41 | 78.0 | 21.84 | | | | | | | | |
| | PLACEBO | 63 | 81.8 | 22.20 | | | | | | | | |
| WEEK 1 | AVP-786 28 mg | 35 | 70.3 | 19.95 | −12.0 | 2.22 | −9.5 | 19.72 | −1.70 | −6.82 | 3.41 | 0.5117 |
| | AVP-786 42.63 mg | 39 | 71.3 | 17.07 | −12.1 | 2.42 | −7.1 | 10.94 | −1.74 | −6.82 | 3.33 | 0.4981 |
| | PLACEBO | 61 | 73.2 | 19.28 | −10.3 | 1.77 | −8.1 | 14.86 | | | | |
| WEEK 2 | AVP-786 28 mg | 34 | 67.2 | 18.82 | −16.4 | 2.40 | −13.4 | 15.90 | −3.37 | −8.96 | 2.22 | 0.2351 |
| | AVP-786 42.63 mg | 37 | 65.8 | 15.69 | −18.3 | 2.57 | −13.8 | 15.18 | −5.22 | −10.8 | 0.30 | 0.0638 |
| | PLACEBO | 59 | 71.3 | 20.11 | −13.0 | 1.91 | −10.9 | 17.53 | | | | |
| WEEK 3 | AVP-786 28 mg | 35 | 67.2 | 19.75 | −15.7 | 2.71 | −13.1 | 19.41 | 0.14 | −6.31 | 6.59 | 0.9653 |
| | AVP-786 42.63 mg | 39 | 63.4 | 16.86 | −18.8 | 2.84 | −14.4 | 14.20 | −2.95 | −9.28 | 3.39 | 0.3594 |
| | PLACEBO | 58 | 67.4 | 18.68 | −15.9 | 2.15 | −14.1 | 20.84 | | | | |
| WEEK 6 | AVP-786 28 mg | 34 | 63.6 | 20.57 | −17.8 | 3.00 | −12.6 | 16.69 | −1.63 | −8.77 | 5.51 | 0.6523 |
| | AVP-786 42.63 mg | 40 | 64.3 | 20.78 | −18.8 | 3.05 | −13.9 | 17.69 | −2.65 | −9.58 | 4.28 | 0.4505 |
| | PLACEBO | 61 | 67.7 | 22.76 | −16.1 | 2.31 | −14.6 | 19.97 | | | | |
| WEEK 9 | AVP-786 28 mg | 28 | 62.9 | 18.36 | −20.5 | 3.10 | −14.0 | 18.11 | 0.46 | −6.87 | 7.79 | 0.9020 |
| | AVP-786 42.63 mg | 40 | 59.2 | 18.09 | −24.2 | 3.06 | −19.0 | 19.03 | −3.18 | −10.2 | 3.79 | 0.3683 |
| | PLACEBO | 59 | 62.7 | 20.90 | −21.0 | 2.34 | −19.6 | 22.20 | | | | |
| WEEK 12 | AVP-786 28 mg | 29 | 66.4 | 21.08 | −18.5 | 3.17 | −11.9 | 19.95 | 1.38 | −6.10 | 8.87 | 0.7149 |
| | AVP-786 42.63 mg | 36 | 58.0 | 17.76 | −26.1 | 3.12 | −21.6 | 16.29 | −6.24 | −13.4 | 0.90 | 0.0861 |
| | PLACEBO | 57 | 64.4 | 20.85 | −19.9 | 2.37 | −18.6 | 21.49 | | | | |

TABLE 68H
SUMMARY OF MEAN CHANGE FROM BASELINE TO WEEK 12 IN CMAI TOTAL SCORE - MMRM,
UN IN FALL RISK ‘NORMAL/MILD’ (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 97 | 74.8 | 19.45 | | | | | | | | |
| | AVP-786 42.63 mg | 99 | 78.3 | 21.82 | | | | | | | | |
| | PLACEBO | 158 | 78.0 | 21.46 | | | | | | | | |
| WEEK 1 | AVP-786 28 mg | 93 | 66.7 | 19.15 | −8.96 | 1.26 | −8.2 | 15.03 | −3.42 | −6.51 | −0.34 | 0.0297 |
| | AVP-786 42.63 mg | 95 | 70.9 | 19.56 | −7.96 | 1.46 | −7.8 | 13.19 | −2.42 | −5.55 | 0.70 | 0.1285 |
| | PLACEBO | 154 | 72.6 | 20.52 | −5.53 | 1.05 | −5.2 | 11.89 | | | | |
| WEEK 2 | AVP-786 28 mg | 82 | 65.0 | 19.45 | −12.2 | 1.41 | −11.0 | 13.80 | −3.70 | −7.17 | −0.24 | 0.0361 |
| | AVP-786 42.63 mg | 92 | 67.8 | 18.88 | −11.2 | 1.57 | −11.1 | 15.54 | −2.76 | −6.21 | 0.70 | 0.1174 |
| | PLACEBO | 140 | 70.1 | 20.58 | −8.46 | 1.16 | −8.8 | 13.81 | | | | |
| WEEK 3 | AVP-786 28 mg | 93 | 61.9 | 19.56 | −14.5 | 1.46 | −13.4 | 14.74 | −4.20 | −7.80 | −0.59 | 0.0227 |
| | AVP-786 42.63 mg | 96 | 65.4 | 19.35 | −12.7 | 1.63 | −12.6 | 14.14 | −2.48 | −6.11 | 1.16 | 0.1809 |
| | PLACEBO | 149 | 67.8 | 19.98 | −10.3 | 1.21 | −10.3 | 17.09 | | | | |
| WEEK 6 | AVP-786 28 mg | 89 | 61.5 | 20.03 | −14.2 | 1.60 | −12.3 | 14.88 | −2.35 | −6.29 | 1.60 | 0.2432 |
| | AVP-786 42.63 mg | 94 | 62.8 | 20.93 | −15.4 | 1.74 | −15.0 | 16.19 | −3.52 | −7.45 | 0.42 | 0.0797 |
| | PLACEBO | 150 | 66.5 | 21.13 | −11.9 | 1.30 | −12.0 | 17.20 | | | | |
| WEEK 9 | AVP-786 28 mg | 76 | 58.9 | 19.67 | −17.6 | 1.63 | −15.5 | 16.05 | −1.27 | −5.26 | 2.72 | 0.5317 |
| | AVP-786 42.63 mg | 93 | 59.7 | 18.87 | −18.7 | 1.73 | −18.5 | 17.92 | −2.32 | −6.24 | 1.61 | 0.2465 |
| | PLACEBO | 146 | 62.0 | 19.21 | −16.4 | 1.30 | −16.6 | 17.78 | | | | |
| WEEK 12 | AVP-786 28 mg | 77 | 61.3 | 21.26 | −16.6 | 1.70 | −14.2 | 18.14 | 0.52 | −3.62 | 4.65 | 0.8056 |
| | AVP-786 42.63 mg | 89 | 57.4 | 18.29 | −20.8 | 1.78 | −20.8 | 17.42 | −3.68 | −7.74 | 0.38 | 0.0753 |
| | PLACEBO | 140 | 61.5 | 18.67 | −17.1 | 1.34 | −17.5 | 18.15 | | | | |

TABLE 68I
SUMMARY OF MEAN CHANGE FROM BASELINE TO WEEK 12 IN CMAI TOTAL SCORE - MMRM,
UN IN PATIENTS AGE >=70 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 72 | 72.9 | 19.77 | | | | | | | | |
| | AVP-786 42.63 mg | 76 | 78.3 | 22.05 | | | | | | | | |
| | PLACEBO | 138 | 78.6 | 21.77 | | | | | | | | |
| WEEK 1 | AVP-786 28 mg | 70 | 65.1 | 18.57 | −9.46 | 1.49 | −7.8 | 16.45 | −3.20 | −6.74 | 0.33 | 0.0757 |
| | AVP-786 42.63 mg | 73 | 71.3 | 20.31 | −7.96 | 1.68 | −6.9 | 12.15 | −1.70 | −5.24 | 1.83 | 0.3441 |
| | PLACEBO | 134 | 72.7 | 20.48 | −6.26 | 1.16 | −5.7 | 12.46 | | | | |
| WEEK 2 | AVP-786 28 mg | 58 | 64.1 | 19.08 | −12.5 | 1.67 | −9.9 | 13.95 | −3.31 | −7.25 | 0.63 | 0.0992 |
| | AVP-786 42.63 mg | 71 | 67.8 | 19.16 | −12.1 | 1.78 | −11.4 | 15.40 | −2.85 | −6.68 | 0.99 | 0.1452 |
| | PLACEBO | 121 | 70.4 | 20.30 | −9.20 | 1.26 | −9.5 | 14.25 | | | | |
| WEEK 3 | AVP-786 28 mg | 69 | 60.7 | 19.08 | −14.7 | 1.75 | −12.6 | 15.06 | −3.61 | −7.80 | 0.57 | 0.0901 |
| | AVP-786 42.63 mg | 74 | 64.8 | 19.78 | −14.1 | 1.89 | −13.5 | 13.52 | −3.04 | −7.18 | 1.09 | 0.1488 |
| | PLACEBO | 129 | 67.8 | 19.90 | −11.1 | 1.34 | −10.8 | 18.19 | | | | |
| WEEK 6 | AVP-786 28 mg | 65 | 61.8 | 19.58 | −12.8 | 1.91 | −10.4 | 15.47 | −0.06 | −4.59 | 4.48 | 0.9804 |
| | AVP-786 42.63 mg | 72 | 63.5 | 21.28 | −15.6 | 2.00 | −14.7 | 16.04 | −2.78 | −7.21 | 1.64 | 0.2170 |
| | PLACEBO | 132 | 66.5 | 21.30 | −12.8 | 1.42 | −12.6 | 17.47 | | | | |
| WEEK 9 | AVP-786 28 mg | 55 | 58.8 | 19.36 | −16.5 | 1.94 | −13.8 | 16.31 | 0.95 | −3.62 | 5.51 | 0.6836 |
| | AVP-786 42.63 mg | 73 | 59.5 | 18.84 | −19.6 | 1.97 | −18.6 | 17.98 | −2.17 | −6.54 | 2.21 | 0.3300 |
| | PLACEBO | 128 | 61.7 | 19.43 | −17.4 | 1.41 | −17.5 | 18.09 | | | | |
| WEEK 12 | AVP-786 28 mg | 56 | 62.0 | 20.96 | −15.5 | 1.99 | −11.8 | 18.65 | 2.40 | −2.30 | 7.11 | 0.3159 |
| | AVP-786 42.63 mg | 67 | 57.3 | 17.84 | −21.8 | 2.03 | −21.3 | 16.82 | −3.87 | −8.40 | 0.66 | 0.0938 |
| | PLACEBO | 121 | 61.3 | 19.14 | −18.0 | 1.45 | −18.4 | 18.64 | | | | |

TABLE 68J
SUMMARY OF MEAN CHANGE FROM BASELINE TO WEEK 12 IN CMAI TOTAL SCORE - MMRM,
UN IN PATIENTS AGE >=80 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 31 | 70.5 | 14.63 | | | | | | | | |
| | AVP-786 42.63 mg | 28 | 74.4 | 22.29 | | | | | | | | |
| | PLACEBO | 67 | 77.3 | 20.43 | | | | | | | | |
| WEEK 1 | AVP-786 28 mg | 30 | 63.6 | 15.34 | −7.92 | 2.27 | −6.9 | 12.72 | −3.21 | −8.61 | 2.20 | 0.2422 |
| | AVP-786 42.63 mg | 28 | 67.5 | 23.69 | −8.52 | 2.68 | −7.0 | 13.18 | −3.81 | −9.53 | 1.90 | 0.1887 |
| | PLACEBO | 64 | 73.0 | 22.22 | −4.71 | 1.62 | −4.3 | 12.19 | | | | |
| WEEK 2 | AVP-786 28 mg | 26 | 62.4 | 17.63 | −10.8 | 2.73 | −9.2 | 14.62 | −3.34 | −9.83 | 3.16 | 0.3112 |
| | AVP-786 42.63 mg | 25 | 63.6 | 25.08 | −13.5 | 3.06 | −12.5 | 16.13 | −6.05 | −12.8 | 0.66 | 0.0766 |
| | PLACEBO | 57 | 70.4 | 23.19 | −7.46 | 1.90 | −8.8 | 12.47 | | | | |
| WEEK 3 | AVP-786 28 mg | 30 | 57.3 | 15.82 | −14.3 | 2.65 | −13.0 | 14.62 | −5.81 | −12.1 | 0.50 | 0.0710 |
| | AVP-786 42.63 mg | 28 | 61.0 | 24.54 | −15.1 | 3.02 | −13.4 | 12.64 | −6.60 | −13.2 | 0.01 | 0.0503 |
| | PLACEBO | 63 | 68.1 | 21.08 | −8.46 | 1.86 | −8.5 | 15.84 | | | | |
| WEEK 6 | AVP-786 28 mg | 28 | 56.8 | 16.65 | −14.5 | 2.95 | −14.3 | 16.63 | −3.89 | −10.9 | 3.10 | 0.2726 |
| | AVP-786 42.63 mg | 27 | 61.7 | 25.00 | −14.1 | 3.27 | −12.0 | 12.60 | −3.51 | −10.7 | 3.72 | 0.3381 |
| | PLACEBO | 65 | 66.9 | 23.38 | −10.6 | 2.02 | −10.6 | 16.85 | | | | |
| WEEK 9 | AVP-786 28 mg | 24 | 57.3 | 17.02 | −15.6 | 2.90 | −14.8 | 18.53 | −0.77 | −7.60 | 6.07 | 0.8247 |
| | AVP-786 42.63 mg | 28 | 56.3 | 22.70 | −19.8 | 3.15 | −18.1 | 13.99 | −4.95 | −11.9 | 2.00 | 0.1608 |
| | PLACEBO | 62 | 62.7 | 21.89 | −14.9 | 1.96 | −15.2 | 15.85 | | | | |
| WEEK 12 | AVP-786 28 mg | 25 | 62.3 | 21.36 | −12.4 | 3.23 | −9.7 | 22.52 | 1.97 | −5.71 | 9.65 | 0.6125 |
| | AVP-786 42.63 mg | 25 | 55.0 | 22.90 | −22.0 | 3.50 | −20.8 | 15.24 | −7.65 | −15.5 | 0.19 | 0.0559 |
| | PLACEBO | 57 | 63.9 | 22.49 | −14.4 | 2.21 | −14.9 | 16.41 | | | | |

TABLE 68K
SUMMARY OF MEAN CHANGE FROM BASELINE TO WEEK 12 IN CMAI TOTAL SCORE - MMRM, UN IN PATIENTS
NOT TAKING BENZODIAZEPINES AT BASELINE (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 91 | 74.0 | 19.73 | | | | | | | | |
| | AVP-786 42.63 mg | 91 | 77.3 | 21.35 | | | | | | | | |
| | PLACEBO | 139 | 76.5 | 20.91 | | | | | | | | |
| WEEK 1 | AVP-786 28 mg | 87 | 65.4 | 18.62 | −9.31 | 1.31 | −8.7 | 15.30 | −3.72 | −6.97 | −0.47 | 0.0251 |
| | AVP-786 42.63 mg | 87 | 69.7 | 19.43 | −7.99 | 1.52 | −7.9 | 13.27 | −2.40 | −5.74 | 0.94 | 0.1578 |
| | PLACEBO | 135 | 71.1 | 20.09 | −5.59 | 1.11 | −5.2 | 12.12 | | | | |
| WEEK 2 | AVP-786 28 mg | 76 | 63.9 | 19.21 | −12.2 | 1.42 | −11.2 | 14.03 | −4.20 | −7.74 | −0.67 | 0.0200 |
| | AVP-786 42.63 mg | 85 | 66.0 | 18.51 | −11.6 | 1.59 | −11.7 | 15.35 | −3.68 | −7.24 | −0.11 | 0.0434 |
| | PLACEBO | 120 | 69.2 | 20.23 | −7.97 | 1.20 | −8.3 | 12.86 | | | | |
| WEEK 3 | AVP-786 28 mg | 87 | 60.5 | 19.22 | −14.9 | 1.48 | −14.0 | 15.56 | −4.84 | −8.54 | −1.13 | 0.0106 |
| | AVP-786 42.63 mg | 88 | 64.0 | 18.45 | −13.0 | 1.67 | −12.9 | 14.01 | −2.89 | −6.66 | 0.89 | 0.1334 |
| | PLACEBO | 129 | 66.6 | 19.64 | −10.1 | 1.25 | −9.9 | 16.41 | | | | |
| WEEK 6 | AVP-786 28 mg | 82 | 60.6 | 19.74 | −14.3 | 1.61 | −12.6 | 16.12 | −2.61 | −6.61 | 1.39 | 0.1997 |
| | AVP-786 42.63 mg | 87 | 61.9 | 19.39 | −15.2 | 1.75 | −15.0 | 16.19 | −3.54 | −7.56 | 0.47 | 0.0834 |
| | PLACEBO | 131 | 65.4 | 20.40 | −11.7 | 1.33 | −11.8 | 15.61 | | | | |
| WEEK 9 | AVP-786 28 mg | 69 | 58.2 | 19.66 | −17.5 | 1.65 | −15.6 | 17.21 | −1.33 | −5.40 | 2.74 | 0.5215 |
| | AVP-786 42.63 mg | 86 | 58.5 | 17.05 | −18.8 | 1.75 | −18.7 | 17.97 | −2.65 | −6.67 | 1.38 | 0.1963 |
| | PLACEBO | 127 | 60.8 | 18.88 | −16.1 | 1.33 | −16.3 | 16.17 | | | | |
| WEEK 12 | AVP-786 28 mg | 70 | 61.4 | 21.82 | −15.7 | 1.75 | −13.6 | 18.77 | 1.10 | −3.22 | 5.42 | 0.6173 |
| | AVP-786 42.63 mg | 81 | 56.3 | 17.21 | −20.9 | 1.83 | −21.0 | 17.64 | −4.14 | −8.39 | 0.12 | 0.0565 |
| | PLACEBO | 120 | 60.4 | 18.36 | −16.8 | 1.41 | −17.2 | 16.97 | | | | |

TABLE 68L
SUMMARY OF MEAN CHANGE FROM BASELINE TO WEEK 12 IN CMAI TOTAL SCORE - MMRM, UN IN
PATIENTS NOT TAKING SNRIS AT BASELINE (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 95 | 74.7 | 19.88 | | | | | | | | |
| | AVP-786 42.63 mg | 96 | 78.0 | 21.30 | | | | | | | | |
| | PLACEBO | 157 | 78.1 | 21.49 | | | | | | | | |
| WEEK 1 | AVP-786 28 mg | 91 | 66.4 | 19.41 | −9.20 | 1.30 | −8.4 | 15.39 | −3.27 | −6.43 | −0.10 | 0.0432 |
| | AVP-786 42.63 mg | 92 | 70.3 | 19.31 | −8.16 | 1.50 | −7.8 | 13.22 | −2.22 | −5.46 | 1.01 | 0.1775 |
| | PLACEBO | 153 | 72.3 | 20.75 | −5.94 | 1.06 | −5.7 | 12.22 | | | | |
| WEEK 2 | AVP-786 28 mg | 80 | 65.3 | 19.87 | −11.8 | 1.43 | −10.6 | 13.12 | −2.65 | −6.13 | 0.83 | 0.1346 |
| | AVP-786 42.63 mg | 89 | 67.0 | 18.57 | −11.9 | 1.58 | −11.6 | 15.79 | −2.69 | −6.19 | 0.81 | 0.1311 |
| | PLACEBO | 139 | 69.7 | 20.79 | −9.17 | 1.15 | −9.5 | 13.88 | | | | |
| WEEK 3 | AVP-786 28 mg | 91 | 61.8 | 19.90 | −14.5 | 1.50 | −13.4 | 14.44 | −3.59 | −7.27 | 0.10 | 0.0564 |
| | AVP-786 42.63 mg | 93 | 64.6 | 18.86 | −13.4 | 1.67 | −13.0 | 14.34 | −2.42 | −6.16 | 1.32 | 0.2046 |
| | PLACEBO | 147 | 67.3 | 20.27 | −11.0 | 1.22 | −10.9 | 17.61 | | | | |
| WEEK 6 | AVP-786 28 mg | 87 | 61.4 | 20.28 | −14.6 | 1.64 | −13.1 | 15.76 | −2.17 | −6.19 | 1.85 | 0.2897 |
| | AVP-786 42.63 mg | 91 | 62.6 | 20.21 | −15.5 | 1.78 | −15.0 | 16.30 | −3.10 | −7.15 | 0.94 | 0.1317 |
| | PLACEBO | 149 | 66.1 | 21.43 | −12.4 | 1.31 | −12.5 | 17.31 | | | | |
| WEEK 9 | AVP-786 28 mg | 74 | 58.9 | 19.92 | −17.9 | 1.67 | −16.4 | 16.64 | −0.88 | −4.95 | 3.18 | 0.6690 |
| | AVP-786 42.63 mg | 90 | 59.3 | 18.08 | −18.9 | 1.77 | −18.6 | 18.05 | −1.96 | −5.98 | 2.07 | 0.3397 |
| | PLACEBO | 145 | 61.5 | 19.58 | −17.0 | 1.31 | −17.3 | 18.15 | | | | |
| WEEK 12 | AVP-786 28 mg | 75 | 61.7 | 21.70 | −16.4 | 1.74 | −14.6 | 18.29 | 1.15 | −3.08 | 5.37 | 0.5944 |
| | AVP-786 42.63 mg | 86 | 57.1 | 17.22 | −21.1 | 1.83 | −20.8 | 17.74 | −3.50 | −7.67 | 0.68 | 0.1002 |
| | PLACEBO | 139 | 61.0 | 19.24 | −17.6 | 1.35 | −18.0 | 18.50 | | | | |

TABLE 68M
SUMMARY OF MEAN CHANGE FROM BASELINE TO WEEK 12 IN CMAI TOTAL SCORE - MMRM, UN IN PATIENTS
BASELINE 12 <= MMSE_BASE <= 24 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 63 | 75.3 | 21.43 | | | | | | | | |
| | AVP-786 42.63 mg | 69 | 77.0 | 20.45 | | | | | | | | |
| | PLACEBO | 113 | 75.9 | 19.84 | | | | | | | | |
| WEEK 1 | AVP-786 28 mg | 60 | 67.2 | 20.37 | −8.41 | 1.59 | −8.3 | 16.63 | −4.38 | −8.17 | −0.59 | 0.0238 |
| | AVP-786 42.63 mg | 66 | 69.7 | 19.93 | −7.37 | 1.75 | −7.5 | 13.21 | −3.34 | −7.11 | 0.44 | 0.0830 |
| | PLACEBO | 111 | 72.1 | 20.61 | −4.04 | 1.25 | −4.0 | 9.99 | | | | |
| WEEK 2 | AVP-786 28 mg | 52 | 65.8 | 20.56 | −11.3 | 1.74 | −11.0 | 14.99 | −3.71 | −7.89 | 0.47 | 0.0818 |
| | AVP-786 42.63 mg | 64 | 67.3 | 19.48 | −9.77 | 1.85 | −9.9 | 14.74 | −2.15 | −6.25 | 1.95 | 0.3018 |
| | PLACEBO | 97 | 68.9 | 20.27 | −7.62 | 1.36 | −8.3 | 12.38 | | | | |
| WEEK 3 | AVP-786 28 mg | 61 | 62.2 | 20.40 | −14.0 | 1.75 | −13.6 | 15.42 | −4.56 | −8.79 | −0.33 | 0.0349 |
| | AVP-786 42.63 mg | 67 | 64.0 | 18.49 | −12.2 | 1.89 | −12.3 | 13.37 | −2.79 | −6.99 | 1.41 | 0.1922 |
| | PLACEBO | 106 | 67.2 | 20.61 | −9.41 | 1.38 | −9.3 | 15.07 | | | | |
| WEEK 6 | AVP-786 28 mg | 59 | 60.9 | 20.60 | −13.5 | 1.81 | −12.2 | 14.06 | −3.26 | −7.64 | 1.13 | 0.1445 |
| | AVP-786 42.63 mg | 65 | 62.6 | 20.75 | −13.7 | 1.94 | −13.6 | 14.91 | −3.42 | −7.75 | 0.90 | 0.1202 |
| | PLACEBO | 106 | 66.3 | 20.31 | −10.3 | 1.42 | −10.4 | 13.97 | | | | |
| WEEK 9 | AVP-786 28 mg | 53 | 57.4 | 18.73 | −17.1 | 1.89 | −15.4 | 14.97 | −1.88 | −6.43 | 2.66 | 0.4149 |
| | AVP-786 42.63 mg | 65 | 59.9 | 18.42 | −16.7 | 1.98 | −16.6 | 16.48 | −1.45 | −5.89 | 2.99 | 0.5204 |
| | PLACEBO | 103 | 61.2 | 19.02 | −15.2 | 1.46 | −15.7 | 15.77 | | | | |
| WEEK 12 | AVP-786 28 mg | 53 | 60.2 | 20.09 | −16.6 | 1.90 | −14.5 | 16.09 | −0.80 | −5.38 | 3.77 | 0.7297 |
| | AVP-786 42.63 mg | 62 | 56.9 | 18.29 | −19.6 | 1.98 | −19.2 | 15.55 | −3.81 | −8.28 | 0.66 | 0.0947 |
| | PLACEBO | 96 | 60.7 | 17.92 | −15.8 | 1.47 | −16.3 | 15.93 | | | | |

TABLE 68N
SUMMARY OF MEAN CHANGE FROM BASELINE TO WEEK 12 IN CMAI TOTAL SCORE - MMRM, UN IN PATIENTS
BASELINE 12 <= MMSE_BASE <= 20 (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 43 | 75.9 | 21.88 | | | | | | | | |
| | AVP-786 42.63 mg | 52 | 76.5 | 18.75 | | | | | | | | |
| | PLACEBO | 76 | 78.0 | 20.93 | | | | | | | | |
| WEEK 1 | AVP-786 28 mg | 40 | 67.2 | 18.91 | −9.27 | 1.98 | −9.1 | 18.13 | −5.64 | −10.5 | −0.81 | 0.0224 |
| | AVP-786 42.63 mg | 50 | 68.9 | 20.15 | −8.01 | 2.12 | −7.6 | 12.46 | −4.38 | −9.06 | 0.30 | 0.0661 |
| | PLACEBO | 74 | 74.4 | 21.37 | −3.63 | 1.56 | −3.8 | 10.90 | | | | |
| WEEK 2 | AVP-786 28 mg | 35 | 64.8 | 19.23 | −12.7 | 2.21 | −13.0 | 16.61 | −4.56 | −9.99 | 0.88 | 0.0996 |
| | AVP-786 42.63 mg | 49 | 67.0 | 19.18 | −10.5 | 2.27 | −10.2 | 14.68 | −2.36 | −7.53 | 2.81 | 0.3676 |
| | PLACEBO | 64 | 70.4 | 21.44 | −8.18 | 1.74 | −9.6 | 13.18 | | | | |
| WEEK 3 | AVP-786 28 mg | 42 | 59.9 | 18.83 | −16.7 | 2.07 | −16.3 | 15.70 | −6.91 | −12.0 | −1.80 | 0.0083 |
| | AVP-786 42.63 mg | 51 | 63.5 | 18.27 | −13.0 | 2.21 | −12.7 | 13.20 | −3.27 | −8.23 | 1.68 | 0.1939 |
| | PLACEBO | 72 | 68.5 | 21.14 | −9.74 | 1.65 | −10.0 | 14.99 | | | | |
| WEEK 6 | AVP-786 28 mg | 39 | 59.1 | 19.86 | −15.9 | 2.39 | −13.6 | 15.92 | −4.92 | −10.8 | 0.92 | 0.0981 |
| | AVP-786 42.63 mg | 50 | 62.7 | 20.74 | −14.0 | 2.41 | −13.3 | 15.18 | −3.06 | −8.61 | 2.49 | 0.2783 |
| | PLACEBO | 72 | 67.3 | 21.06 | −10.9 | 1.84 | −11.2 | 14.83 | | | | |
| WEEK 9 | AVP-786 28 mg | 35 | 55.6 | 19.03 | −18.8 | 2.42 | −16.1 | 16.19 | −3.45 | −9.37 | 2.48 | 0.2522 |
| | AVP-786 42.63 mg | 51 | 60.1 | 17.93 | −16.8 | 2.41 | −15.9 | 16.19 | −1.45 | −7.02 | 4.12 | 0.6086 |
| | PLACEBO | 69 | 62.9 | 20.31 | −15.4 | 1.86 | −16.2 | 16.39 | | | | |
| WEEK 12 | AVP-786 28 mg | 36 | 60.5 | 20.63 | −17.3 | 2.39 | −13.3 | 17.18 | −2.02 | −7.90 | 3.85 | 0.4977 |
| | AVP-786 42.63 mg | 47 | 56.9 | 18.07 | −20.2 | 2.41 | −19.0 | 14.96 | −4.93 | −10.5 | 0.65 | 0.0830 |
| | PLACEBO | 64 | 63.3 | 18.79 | −15.2 | 1.86 | −16.3 | 16.13 | | | | |

TABLE 68O
SUMMARY OF MEAN CHANGE FROM BASELINE TO WEEK 12 IN CMAI TOTAL SCORE - MMRM, UN IN
PATIENTS BASELINE MMSE >=17 MEDIAN (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 47 | 69.3 | 17.98 | | | | | | | | |
| | AVP-786 42.63 mg | 56 | 76.7 | 20.50 | | | | | | | | |
| | PLACEBO | 87 | 72.4 | 19.04 | | | | | | | | |
| WEEK 1 | AVP-786 28 mg | 45 | 63.1 | 19.73 | −6.45 | 1.67 | −6.3 | 10.73 | −2.78 | −6.66 | 1.10 | 0.1591 |
| | AVP-786 42.63 mg | 55 | 69.5 | 19.28 | −6.23 | 1.77 | −7.5 | 13.19 | −2.55 | −6.31 | 1.21 | 0.1822 |
| | PLACEBO | 86 | 68.2 | 20.33 | −3.68 | 1.30 | −4.0 | 9.60 | | | | |
| WEEK 2 | AVP-786 28 mg | 41 | 61.0 | 18.71 | −9.76 | 1.94 | −9.0 | 11.79 | −2.60 | −7.20 | 2.01 | 0.2671 |
| | AVP-786 42.63 mg | 50 | 66.4 | 20.79 | −8.93 | 2.00 | −10.8 | 14.23 | −1.77 | −6.20 | 2.66 | 0.4318 |
| | PLACEBO | 76 | 65.0 | 18.50 | −7.16 | 1.51 | −8.2 | 11.93 | | | | |
| WEEK 3 | AVP-786 28 mg | 45 | 59.1 | 20.67 | −11.4 | 2.03 | −10.7 | 14.16 | −2.50 | −7.32 | 2.33 | 0.3085 |
| | AVP-786 42.63 mg | 54 | 63.5 | 19.60 | −10.7 | 2.07 | −12.4 | 12.67 | −1.79 | −6.43 | 2.86 | 0.4498 |
| | PLACEBO | 81 | 63.8 | 19.43 | −8.92 | 1.56 | −9.2 | 15.08 | | | | |
| WEEK 6 | AVP-786 28 mg | 45 | 59.2 | 21.24 | −10.7 | 2.00 | −9.9 | 12.38 | −1.67 | −6.43 | 3.10 | 0.4907 |
| | AVP-786 42.63 mg | 53 | 61.9 | 21.80 | −13.0 | 2.05 | −13.9 | 14.96 | −3.93 | −8.51 | 0.66 | 0.0927 |
| | PLACEBO | 83 | 63.5 | 19.43 | −9.04 | 1.55 | −9.6 | 12.79 | | | | |
| WEEK 9 | AVP-786 28 mg | 39 | 57.5 | 20.46 | −14.3 | 2.14 | −12.6 | 14.68 | −0.12 | −5.20 | 4.95 | 0.9613 |
| | AVP-786 42.63 mg | 53 | 58.5 | 19.44 | −16.1 | 2.13 | −18.1 | 14.91 | −1.94 | −6.76 | 2.87 | 0.4270 |
| | PLACEBO | 80 | 58.4 | 17.71 | −14.2 | 1.62 | −14.9 | 15.61 | | | | |
| WEEK 12 | AVP-786 28 mg | 39 | 57.9 | 20.69 | −13.8 | 2.20 | −12.4 | 15.17 | 0.95 | −4.29 | 6.18 | 0.7221 |
| | AVP-786 42.63 mg | 51 | 54.6 | 18.57 | −19.5 | 2.17 | −21.5 | 15.04 | −4.80 | −9.74 | 0.15 | 0.0574 |
| | PLACEBO | 74 | 57.9 | 18.25 | −14.7 | 1.67 | −15.0 | 15.82 | | | | |

TABLE 68P
SUMMARY OF MEAN CHANGE FROM BASELINE TO WEEK 12 IN CMAI TOTAL
SCORE - MMRM, UN (MITT POPULATION, BASELINE FACTOR 1 PATIENTS)

| | TREATMENT | | | | CHANGE FROM BASELINE | | | | | 95% CI | | |
|---|---|---|---|---|---|---|---|---|---|---|---|---|
| VISIT | GROUP | N | MEAN | SD | LSMEAN¹ | SE¹ | MEAN | SD | DIFF¹ | LOWER¹ | UPPER¹ | P-VALUE¹ |
| BASELINE | AVP-786 28 mg | 99 | 74.8 | 19.90 | | | | | | | | |
| | AVP-786 42.63 mg | 104 | 77.8 | 21.52 | | | | | | | | |
| | PLACEBO | 166 | 77.6 | 21.17 | | | | | | | | |
| WEEK 1 | AVP-786 28 mg | 95 | 66.4 | 19.25 | −9.13 | 1.25 | −8.4 | 15.12 | −3.29 | −6.31 | −0.26 | 0.0334 |
| | AVP-786 42.63 mg | 99 | 70.3 | 19.45 | −8.08 | 1.42 | −7.7 | 13.01 | −2.23 | −5.29 | 0.82 | 0.1517 |
| | PLACEBO | 162 | 71.9 | 20.31 | −5.84 | 1.02 | −5.6 | 12.00 | | | | |
| WEEK 2 | AVP-786 28 mg | 84 | 64.8 | 19.52 | −12.2 | 1.39 | −11.1 | 13.83 | −3.37 | −6.74 | 0.01 | 0.0504 |
| | AVP-786 42.63 mg | 97 | 67.0 | 18.90 | −11.5 | 1.52 | −11.4 | 15.24 | −2.75 | −6.09 | 0.60 | 0.1070 |
| | PLACEBO | 147 | 69.5 | 20.33 | −8.79 | 1.12 | −9.1 | 13.78 | | | | |
| WEEK 3 | AVP-786 28 mg | 95 | 61.6 | 19.52 | −14.6 | 1.45 | −13.6 | 15.04 | −3.97 | −7.53 | −0.40 | 0.0293 |
| | AVP-786 42.63 mg | 101 | 64.6 | 19.27 | −13.2 | 1.59 | −12.9 | 13.97 | −2.50 | −6.07 | 1.06 | 0.1681 |
| | PLACEBO | 156 | 67.1 | 19.87 | −10.7 | 1.18 | −10.6 | 17.29 | | | | |
| WEEK 6 | AVP-786 28 mg | 90 | 61.3 | 19.96 | −14.5 | 1.59 | −12.9 | 15.56 | −2.40 | −6.29 | 1.48 | 0.2247 |
| | AVP-786 42.63 mg | 99 | 62.7 | 20.73 | −15.1 | 1.69 | −14.7 | 15.95 | −3.00 | −6.85 | 0.84 | 0.1248 |
| | PLACEBO | 158 | 66.0 | 20.92 | −12.1 | 1.26 | −12.2 | 17.07 | | | | |
| WEEK 9 | AVP-786 28 mg | 77 | 58.9 | 19.55 | −17.7 | 1.62 | −16.0 | 16.48 | −1.08 | −5.01 | 2.84 | 0.5881 |
| | AVP-786 42.63 mg | 98 | 59.4 | 18.75 | −18.6 | 1.69 | −18.3 | 17.53 | −1.92 | −5.74 | 1.91 | 0.3246 |
| | PLACEBO | 154 | 61.3 | 19.15 | −16.7 | 1.26 | −16.9 | 17.78 | | | | |
| WEEK 12 | AVP-786 28 mg | 78 | 61.6 | 21.31 | −16.3 | 1.68 | −14.3 | 18.03 | 1.14 | −2.93 | 5.21 | 0.5837 |
| | AVP-786 42.63 mg | 92 | 57.2 | 18.25 | −20.8 | 1.74 | −20.7 | 17.28 | −3.34 | −7.30 | 0.62 | 0.0981 |
| | PLACEBO | 147 | 60.8 | 18.82 | −17.4 | 1.30 | −17.9 | 18.06 | | | | |
| | AVERAGE | | | | | | | | −1.10 | −4.41 | 2.20 | 0.5120 |

TABLE 68Q
SUMMARY OF MEAN CHANGE FROM BASELINE TO WEEK 12 IN CMAI TOTAL SCORE - MMRM,
UN IN MALES (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 46 | 75.6 | 20.15 | | | | | | | | |
| | AVP-786 | 42.63 | mg | 44 | 78.8 | 24.58 | | | | | | | | |
| | PLACEBO | | | 75 | 73.9 | 19.69 | | | | | | | | |
| WEEK 1 | AVP-786 | 28 | mg | 43 | 64.1 | 16.94 | −11.2 | 1.91 | −11.7 | 18.98 | −7.25 | −12.1 | −2.45 | 0.0033 |
| | AVP-786 | 42.63 | mg | 42 | 70.3 | 19.44 | −7.56 | 2.22 | −8.9 | 13.15 | −3.58 | −8.51 | 1.36 | 0.1546 |
| | PLACEBO | | | 73 | 70.5 | 19.63 | −3.98 | 1.57 | −3.3 | 12.31 | | | | |
| WEEK 2 | AVP-786 | 28 | mg | 39 | 62.0 | 17.74 | −13.8 | 1.94 | −13.6 | 16.51 | −6.40 | −11.3 | −1.51 | 0.0107 |
| | AVP-786 | 42.63 | mg | 41 | 67.3 | 17.27 | −10.9 | 2.22 | −12.8 | 15.41 | −3.45 | −8.41 | 1.51 | 0.1709 |
| | PLACEBO | | | 67 | 67.1 | 18.30 | −7.43 | 1.60 | 7.7 | 13.59 | | | | |
| WEEK 3 | AVP-786 | 28 | mg | 43 | 60.5 | 16.98 | −15.6 | 1.93 | −15.7 | 16.46 | −6.82 | −11.7 | −1.94 | 0.0065 |
| | AVP-786 | 42.63 | mg | 43 | 64.3 | 18.31 | −13.1 | 2.23 | −14.8 | 13.22 | −4.34 | −9.34 | 0.66 | 0.0883 |
| | PLACEBO | | | 70 | 66.1 | 19.05 | −8.74 | 1.60 | −7.7 | 16.09 | | | | |
| WEEK 6 | AVP-786 | 28 | mg | 41 | 60.4 | 16.38 | −15.0 | 2.23 | −13.5 | 15.50 | −5.79 | −11.4 | −0.21 | 0.0421 |
| | AVP-786 | 42.63 | mg | 43 | 59.6 | 19.44 | −18.0 | 2.46 | −19.5 | 15.64 | −8.76 | −14.4 | −3.15 | 0.0024 |
| | PLACEBO | | | 72 | 65.0 | 21.61 | −9.23 | 1.78 | −8.8 | 15.90 | | | | |
| WEEK 9 | AVP-786 | 28 | mg | 30 | 58.4 | 17.32 | −19.0 | 2.37 | −18.4 | 15.79 | −3.61 | −9.45 | 2.24 | 0.2249 |
| | AVP-786 | 42.63 | mg | 41 | 57.3 | 17.30 | −19.3 | 2.51 | −21.5 | 19.74 | −3.90 | −9.65 | 1.84 | 0.1815 |
| | PLACEBO | | | 71 | 59.1 | 18.56 | −15.4 | 1.81 | −14.3 | 17.50 | | | | |
| WEEK 12 | AVP-786 | 28 | mg | 32 | 59.6 | 19.03 | −17.1 | 2.60 | −15.3 | 16.77 | −2.45 | −8.31 | 3.91 | 0.4479 |
| | AVP-786 | 42.63 | mg | 38 | 55.7 | 18.57 | −21.2 | 2.68 | −23.4 | 19.43 | −6.53 | −12.7 | −0.34 | 0.0389 |

| | | | | | | | |
|---|---|---|---|---|---|---|---|
| PLACEBO | 67 | 60.1 | 18.63 | −14.6 | 1.94 | −13.3 | 18.48 |

TABLE 68R
SUMMARY OF MEAN CHANGE FROM BASELINE TO WEEK 12 IN CMAI TOTAL SCORE - MMRM,
UN IN BASELINE CMAI <65 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 33 | 56.5 | 6.03 | | | | | | | | |
| | AVP-786 | 42.63 | mg | 31 | 56.4 | 6.01 | | | | | | | | |
| | PLACEBO | | | 49 | 56.3 | 6.03 | | | | | | | | |
| WEEK 1 | AVP-786 | 28 | mg | 33 | 51.7 | 10.71 | −5.00 | 1.31 | −4.7 | 9.48 | −2.36 | −5.68 | 0.96 | 0.1614 |
| | AVP-786 | 42.63 | mg | 30 | 53.1 | 7.27 | −4.11 | 1.58 | −3.2 | 6.23 | −1.47 | −5.04 | 2.09 | 0.4150 |
| | PLACEBO | | | 48 | 54.1 | 8.43 | −2.64 | 1.10 | −2.3 | 6.56 | | | | |
| WEEK 2 | AVP-786 | 28 | mg | 28 | 49.4 | 9.40 | −7.95 | 1.59 | −7.4 | 9.44 | −4.80 | −8.83 | −0.77 | 0.0200 |
| | AVP-786 | 42.63 | mg | 28 | 51.8 | 10.16 | −5.70 | 1.80 | −4.6 | 8.19 | −2.54 | −6.71 | 1.63 | 0.2289 |
| | F1ACEBO | | | 42 | 53.2 | 8.44 | −3.15 | 1.33 | −2.9 | 8.94 | | | | |
| WEEK 3 | AVP-786 | 28 | mg | 31 | 47.8 | 11.98 | −9.37 | 1.85 | −8.6 | 11.84 | −6.96 | −11.7 | −2.26 | 0.0041 |
| | AVP-786 | 42.63 | mg | 31 | 49.6 | 9.73 | −7.79 | 2.04 | −6.8 | 9.11 | −5.38 | −10.2 | −0.51 | 0.0308 |
| | PLACEBO | | | 46 | 54.2 | 11.41 | −2.41 | 1.53 | −2.2 | 10.16 | | | | |
| WEEK 6 | AVP-786 | 28 | mg | 29 | 51.6 | 16.99 | −5.70 | 2.07 | −5.1 | 16.14 | −3.52 | −8.74 | 1.70 | 0.1837 |
| | AVP-786 | 42.63 | mg | 31 | 46.3 | 7.54 | −11.1 | 2.20 | −10.1 | 7.84 | −8.90 | −14.2 | −3.60 | 0.0012 |
| | PLACEBO | | | 46 | 54.6 | 10.35 | −2.18 | 1.67 | −1.9 | 9.58 | | | | |
| WEEK 9 | AVP-786 | 28 | mg | 24 | 48.3 | 14.44 | −9.87 | 2.01 | −8.4 | 14.11 | −4.13 | −9.15 | 0.89 | 0.1062 |
| | AVP-786 | 42.63 | mg | 30 | 46.0 | 7.44 | −11.2 | 2.08 | −10.3 | 6.65 | −5.42 | −10.4 | −0.43 | 0.0334 |
| | PLACEBO | | | 44 | 51.3 | 11.42 | −5.74 | 1.53 | −5.0 | 10.04 | | | | |
| WEEK 12 | AVP-786 | 28 | mg | 24 | 50.5 | 19.13 | −7.42 | 2.53 | −6.8 | 18.60 | −1.13 | −7.51 | 5.25 | 0.7259 |
| | AVP-786 | 42.63 | mg | 29 | 44.9 | 8.95 | −12.6 | 2.55 | −11.4 | 8.60 | −6.29 | −12.6 | 0.01 | 0.0502 |

| | | | | | | | |
|---|---|---|---|---|---|---|---|
| PLACEBO | 40 | 50.6 | 12.63 | −6.29 | 2.02 | −5.7 | 11.83 |

TABLE 68S
SUMMARY OF MEAN CHANGE FROM BASELINE TO WEEK 12 IN CMAI TOTAL SCORE - MMRM,
UN IN PATIENTS AGE >=55 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 98 | 75.0 | 19.89 | | | | | | | | |
| | AVP-786 | 42.63 | mg | 104 | 77.8 | 21.52 | | | | | | | | |
| | PLACEBO | | | 164 | 77.7 | 21.29 | | | | | | | | |
| WEEK 1 | AVP-786 | 28 | mg | 94 | 66.6 | 19.22 | −9.08 | 1.26 | −8.4 | 15.20 | −3.22 | −6.28 | −0.17 | 0.0387 |
| | AVP-786 | 42.63 | mg | 99 | 70.3 | 19.45 | −8.06 | 1.43 | −7.7 | 13.01 | −2.21 | −5.28 | 0.86 | 0.1580 |
| | PLACEBO | | | 160 | 72.0 | 20.43 | −5.85 | 1.03 | −5.6 | 12.05 | | | | |
| WEEK 2 | AVP-786 | 28 | mg | 83 | 65.2 | 19.39 | −12.0 | 1.39 | −11.0 | 13.90 | −3.27 | −6.66 | 0.13 | 0.0593 |
| | AVP-786 | 42.63 | mg | 97 | 67.0 | 18.90 | −11.5 | 1.52 | −11.4 | 15.24 | −2.76 | −6.11 | 0.60 | 0.1068 |
| | PLACEBO | | | 146 | 69.5 | 20.40 | −8.77 | 1.13 | −9.2 | 13.80 | | | | |
| WEEK 3 | AVP-786 | 28 | mg | 94 | 61.9 | 19.42 | −14.5 | 1.46 | −13.6 | 15.11 | −3.88 | −7.47 | −0.29 | 0.0343 |
| | AVP-786 | 42.63 | mg | 101 | 64.6 | 19.27 | −13.2 | 1.60 | −12.9 | 13.97 | −2.52 | −6.10 | 1.06 | 0.1669 |
| | PLACEBO | | | 154 | 67.2 | 19.97 | −10.6 | 1.19 | −10.5 | 17.34 | | | | |
| WEEK 6 | AVP-786 | 28 | mg | 89 | 61.6 | 19.96 | −14.5 | 1.60 | −12.9 | 15.65 | −2.24 | −6.16 | 1.68 | 0.2617 |
| | AVP-786 | 42.63 | mg | 99 | 62.7 | 20.73 | −15.1 | 1.70 | −14.7 | 15.95 | −2.89 | −6.75 | 0.97 | 0.1423 |
| | PLACEBO | | | 156 | 65.9 | 21.04 | −12.2 | 1.28 | −12.3 | 17.14 | | | | |
| WEEK 9 | AVP-786 | 28 | mg | 76 | 59.1 | 19.54 | −17.7 | 1.63 | −16.0 | 16.59 | −1.10 | −5.06 | 2.85 | 0.5836 |
| | AVP-786 | 42.63 | mg | 98 | 59.4 | 18.75 | −18.6 | 1.69 | −18.3 | 17.53 | −1.99 | −5.83 | 1.85 | 0.3083 |
| | PLACEBO | | | 152 | 61.5 | 19.21 | −16.6 | 1.27 | −16.8 | 17.81 | | | | |
| WEEK 12 | AVP-786 | 28 | mg | 77 | 61.9 | 21.30 | −16.2 | 1.69 | −14.3 | 18.15 | 1.07 | −3.03 | 5.16 | 0.6093 |
| | AVP-786 | 42.63 | mg | 92 | 57.2 | 18.25 | −20.8 | 1.74 | −20.7 | 17.28 | −3.47 | −7.45 | 0.50 | 0.0865 |

| | | | | | | | |
|---|---|---|---|---|---|---|---|
| PLACEBO | 145 | 61.0 | 18.85 | −17.3 | 1.31 | −17.8 | 18.08 |

TABLE 68T
SUMMARY OF MEAN CHANGE FROM BASELINE TO WEEK 12 IN CMAI TOTAL SCORE - MMRM, UN IN
PATIENTS BASELINE 8 <= MMSE_BASE <=24 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 87 | 76.0 | 20.37 | | | | | | | | |
| | AVP-786 | 42.63 | mg | 89 | 78.7 | 21.29 | | | | | | | | |
| | PLACEBO | | | 142 | 78.0 | 20.76 | | | | | | | | |
| WEEK 1 | AVP-786 | 28 | mg | 84 | 67.2 | 19.71 | −9.57 | 1.36 | −9.0 | 15.41 | −4.26 | −7.56 | −0.95 | 0.0117 |
| | AVP-786 | 42.63 | mg | 84 | 70.4 | 19.28 | −8.64 | 1.55 | −8.6 | 13.66 | −3.33 | −6.70 | 0.04 | 0.0526 |
| | PLACEBO | | | 139 | 72.8 | 19.73 | −5.31 | 1.12 | −5.1 | 12.13 | | | | |
| WEEK 2 | AVP-786 | 28 | mg | 72 | 66.1 | 20.14 | −12.3 | 1.52 | −11.5 | 14.20 | −3.29 | −6.99 | 0.42 | 0.0822 |
| | AVP-786 | 42.63 | mg | 84 | 67.3 | 17.92 | −11.6 | 1.66 | −11.7 | 16.22 | −2.66 | −6.36 | 1.04 | 0.1580 |
| | PLACEBO | | | 125 | 69.6 | 19.65 | −8.97 | 1.24 | −9.2 | 13.95 | | | | |
| WEEK 3 | AVP-786 | 28 | mg | 83 | 62.3 | 19.85 | −15.1 | 1.55 | −14.3 | 14.64 | −4.40 | −8.20 | −0.61 | 0.0231 |
| | AVP-786 | 42.63 | mg | 96 | 64.5 | 18.28 | −13.8 | 1.71 | −13.9 | 14.57 | −3.18 | −7.01 | 0.65 | 0.1031 |
| | PLACEBO | | | 134 | 67.9 | 19.98 | −10.7 | 1.27 | −10.4 | 17.26 | | | | |
| WEEK 6 | AVP-786 | 28 | mg | 79 | 61.2 | 20.03 | −15.3 | 1.64 | −13.7 | 14.06 | −3.79 | −7.80 | 0.23 | 0.0643 |
| | AVP-786 | 42.63 | mg | 84 | 62.5 | 20.04 | −15.9 | 1.77 | −15.8 | 16.31 | −4.48 | −8.48 | −0.47 | 0.0286 |
| | PLACEBO | | | 134 | 67.0 | 20.50 | −11.5 | 1.32 | −11.6 | 16.32 | | | | |
| WEEK 9 | AVP-786 | 28 | mg | 69 | 59.0 | 19.23 | −18.4 | 1.67 | −16.3 | 15.31 | −1.88 | −5.94 | 2.18 | 0.3628 |
| | AVP-786 | 42.63 | mg | 84 | 59.7 | 17.85 | −18.8 | 1.77 | −18.8 | 18.32 | −2.29 | −6.30 | 1.71 | 0.2611 |
| | PLACEBO | | | 130 | 61.9 | 18.42 | −16.5 | 1.32 | −16.8 | 17.17 | | | | |
| WEEK 12 | AVP-786 | 28 | mg | 69 | 61.8 | 20.63 | −17.1 | 1.70 | −15.0 | 16.00 | −0.25 | −4.35 | 3.84 | 0.9033 |
| | AVP-786 | 42.63 | mg | 79 | 57.2 | 17.94 | −21.2 | 1.78 | −21.2 | 18.01 | −4.35 | −8.38 | −0.32 | 0.0344 |

| | | | | | | | |
|---|---|---|---|---|---|---|---|
| PLACEBO | 124 | 61.6 | 17.47 | −16.9 | 1.33 | −17.5 | 16.92 |

TABLE 68U
SUMMARY OF MEAN CHANGE FROM BASELINE TO WEEK 12 IN CMAI TOTAL SCORE - MMRM, UN IN
PATIENTS BASELINE 8 <= MMSE_BASE <=22 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 79 | 76.1 | 20.51 | | | | | | | | |
| | AVP-786 | 42.63 | mg | 76 | 80.0 | 21.01 | | | | | | | | |
| | PLACEBO | | | 121 | 78.8 | 21.12 | | | | | | | | |
| WEEK 1 | AVP-786 | 28 | mg | 76 | 66.6 | 19.84 | −10.4 | 1.49 | −9.7 | 15.93 | −4.72 | −8.43 | −1.02 | 0.0127 |
| | AVP-786 | 42.63 | mg | 72 | 70.6 | 19.68 | −9.34 | 1.75 | −9.6 | 14.32 | −3.64 | −7.47 | 0.19 | 0.0624 |
| | PLACEBO | | | 118 | 73.2 | 19.83 | −5.70 | 1.27 | −5.6 | 13.00 | | | | |
| WEEK 2 | AVP-786 | 28 | mg | 65 | 65.3 | 20.03 | −13.6 | 1.65 | −12.6 | 14.45 | −3.98 | −8.08 | 0.13 | 0.0574 |
| | AVP-786 | 42.63 | mg | 73 | 67.8 | 17.98 | −12.3 | 1.85 | −12.8 | 17.01 | −2.69 | −6.84 | 1.45 | 0.2021 |
| | PLACEBO | | | 108 | 69.6 | 19.62 | −9.60 | 1.39 | −10.0 | 14.75 | | | | |
| WEEK 3 | AVP-786 | 28 | mg | 75 | 61.2 | 19.78 | −16.5 | 1.68 | −15.6 | 14.66 | −5.07 | −9.26 | −0.88 | 0.0178 |
| | AVP-786 | 42.63 | mg | 74 | 65.1 | 18.78 | −14.3 | 1.91 | −14.9 | 15.23 | −2.84 | −7.13 | 1.44 | 0.1925 |
| | PLACEBO | | | 114 | 67.9 | 20.03 | −11.4 | 1.42 | −11.3 | 18.22 | | | | |
| WEEK 6 | AVP-786 | 28 | mg | 71 | 60.5 | 19.96 | −16.2 | 1.78 | −14.5 | 14.26 | −3.86 | −8.26 | 0.55 | 0.0861 |
| | AVP-786 | 42.63 | mg | 73 | 63.7 | 20.60 | −15.9 | 1.96 | −16.2 | 16.78 | −3.57 | −8.02 | 0.88 | 0.1149 |
| | PLACEBO | | | 115 | 66.9 | 20.53 | −12.3 | 1.47 | −12.5 | 17.00 | | | | |
| WEEK 9 | AVP-786 | 28 | mg | 62 | 58.9 | 19.65 | −19.0 | 1.81 | −16.5 | 15.49 | −1.59 | −6.06 | 2.88 | 0.4838 |
| | AVP-786 | 42.63 | mg | 74 | 60.8 | 18.18 | −18.8 | 1.96 | −19.1 | 18.81 | −1.37 | −5.83 | 3.09 | 0.5461 |
| | PLACEBO | | | 111 | 61.8 | 18.69 | −17.4 | 1.48 | −17.7 | 17.76 | | | | |
| WEEK 12 | AVP-786 | 28 | mg | 63 | 62.3 | 20.86 | −17.0 | 1.82 | −14.2 | 16.31 | 0.33 | −4.14 | 4.79 | 0.8859 |
| | AVP-786 | 42.63 | mg | 68 | 58.3 | 18.39 | −21.3 | 1.97 | −21.9 | 18.41 | −3.94 | −8.41 | 0.52 | 0.0832 |

| | | | | | | | |
|---|---|---|---|---|---|---|---|
| PLACEBO | 107 | 61.9 | 17.63 | −17.4 | 1.47 | −18.2 | 17.29 |

TABLE 68V
SUMMARY OF MEAN CHANGE FROM BASELINE TO WEEK 12 IN CMAI TOTAL SCORE - MMRM, UN IN
PATIENTS BASELINE 6 <= MMSE_BASE <=20 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 74 | 76.3 | 19.58 | | | | | | | | |
| | AVP-786 | 42.63 | mg | 76 | 78.8 | 20.96 | | | | | | | | |
| | PLACEBO | | | 119 | 80.4 | 21.91 | | | | | | | | |
| WEEK 1 | AVP-786 | 28 | mg | 70 | 67.2 | 17.84 | −10.1 | 1.50 | −9.3 | 15.77 | −3.99 | −7.70 | −0.29 | 0.0347 |
| | AVP-786 | 42.63 | mg | 72 | 70.4 | 19.89 | −9.18 | 1.73 | −8.5 | 13.02 | −3.11 | −6.89 | 0.66 | 0.1055 |
| | PLACEBO | | | 115 | 74.3 | 20.46 | −6.06 | 1.24 | −6.0 | 13.43 | | | | |
| WEEK 2 | AVP-786 | 28 | mg | 62 | 65.5 | 18.37 | −13.3 | 1.69 | −12.4 | 14.77 | −3.55 | −7.75 | 0.65 | 0.0971 |
| | AVP-786 | 42.63 | mg | 73 | 67.4 | 18.36 | −12.6 | 1.86 | −11.9 | 16.10 | −2.79 | −6.97 | 1.39 | 0.1897 |
| | PLACEBO | | | 105 | 71.1 | 21.03 | −9.76 | 1.38 | −10.6 | 14.86 | | | | |
| WEEK 3 | AVP-786 | 28 | mg | 71 | 61.2 | 18.28 | −16.6 | 1.72 | −15.4 | 15.18 | −5.20 | −9.50 | −0.89 | 0.0182 |
| | AVP-786 | 42.63 | mg | 74 | 64.8 | 19.53 | −14.3 | 1.92 | −13.9 | 14.62 | −2.98 | −7.32 | 1.36 | 0.1781 |
| | PLACEBO | | | 113 | 68.5 | 19.91 | −11.4 | 1.42 | −11.6 | 18.32 | | | | |
| WEEK 6 | AVP-786 | 28 | mg | 66 | 61.2 | 19.09 | −16.2 | 1.97 | −13.9 | 16.57 | −3.35 | −8.23 | 1.53 | 0.1775 |
| | AVP-786 | 42.63 | mg | 73 | 63.5 | 21.21 | −15.9 | 2.10 | −15.1 | 16.84 | −2.99 | −7.83 | 1.85 | 0.2245 |
| | PLACEBO | | | 114 | 67.6 | 21.41 | −12.9 | 1.57 | −13.3 | 18.75 | | | | |
| WEEK 9 | AVP-786 | 28 | mg | 56 | 58.9 | 19.84 | −19.1 | 1.99 | −16.7 | 17.33 | −1.98 | −6.88 | 2.92 | 0.4262 |
| | AVP-786 | 42.63 | mg | 73 | 60.5 | 18.85 | −19.0 | 2.07 | −18.2 | 18.34 | −1.83 | −6.61 | 2.95 | 0.4514 |
| | PLACEBO | | | 110 | 63.3 | 19.82 | −17.1 | 1.56 | −17.7 | 19.03 | | | | |
| WEEK 12 | AVP-786 | 28 | mg | 57 | 63.1 | 21.83 | −16.8 | 2.07 | −13.7 | 19.14 | 0.48 | −4.60 | 5.56 | 0.8528 |
| | AVP-786 | 42.63 | mg | 67 | 58.1 | 18.53 | −21.5 | 2.15 | −21.0 | 18.03 | −4.25 | −9.22 | 0.72 | 0.0932 |

| | | | | | | | |
|---|---|---|---|---|---|---|---|
| PLACEBO | 106 | 63.3 | 19.46 | −17.2 | 1.61 | −18.4 | 19.27 |

TABLE 68W
SUMMARY OF MEAN CHANGE FROM BASELINE TO WEEK 12 IN CMAI TOTAL SCORE - MMRM, UN IN
PATIENTS BASELINE 8 <= MMSE_BASE <=20 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 67 | 76.6 | 20.32 | | | | | | | | |
| | AVP-786 | 42.63 | mg | 72 | 78.8 | 20.35 | | | | | | | | |
| | PLACEBO | | | 105 | 80.2 | 21.61 | | | | | | | | |
| WEEK 1 | AVP-786 | 28 | mg | 64 | 67.2 | 18.57 | −10.5 | 1.58 | −9.8 | 16.01 | −5.16 | −9.07 | −1.25 | 0.0099 |
| | AVP-786 | 42.63 | mg | 68 | 70.0 | 19.32 | −9.59 | 1.78 | −8.9 | 13.22 | −4.22 | −8.15 | −0.28 | 0.0357 |
| | PLACEBO | | | 182 | 74.8 | 19.92 | −5.38 | 1.32 | −5.4 | 13.34 | | | | |
| WEEK 2 | AVP-786 | 28 | mg | 55 | 65.6 | 19.17 | −13.6 | 1.79 | −12.9 | 15.03 | −3.83 | −8.29 | 0.63 | 0.0919 |
| | AVP-786 | 42.63 | mg | 69 | 67.1 | 17.32 | −12.8 | 1.92 | −12.3 | 16.44 | −3.08 | −7.45 | 1.29 | 0.1661 |
| | PLACEBO | | | 92 | 71.0 | 20.22 | −9.74 | 1.48 | −10.4 | 14.90 | | | | |
| WEEK 3 | AVP-786 | 28 | mg | 64 | 60.8 | 18.68 | −17.2 | 1.75 | −16.2 | 14.54 | −5.99 | −10.4 | −1.62 | 0.0074 |
| | AVP-786 | 42.63 | mg | 70 | 64.2 | 18.08 | −15.0 | 1.92 | −14.6 | 14.65 | −3.76 | −8.11 | 0.59 | 0.0900 |
| | PLACEBO | | | 100 | 69.1 | 20.12 | −11.2 | 1.46 | −11.3 | 17.85 | | | | |
| WEEK 6 | AVP-786 | 28 | mg | 59 | 60.1 | 19.37 | −17.3 | 1.98 | −15.2 | 15.15 | −4.98 | −9.89 | −0.08 | 0.0464 |
| | AVP-786 | 42.63 | mg | 69 | 62.5 | 19.88 | −16.8 | 2.07 | −16.1 | 16.78 | −4.50 | −9.28 | 0.28 | 0.0651 |
| | PLACEBO | | | 100 | 68.1 | 21.06 | −12.3 | 1.60 | −12.6 | 17.50 | | | | |
| WEEK 9 | AVP-786 | 28 | mg | 51 | 58.3 | 19.73 | −20.0 | 2.00 | −17.2 | 16.20 | −2.99 | −7.92 | 1.94 | 0.2330 |
| | AVP-786 | 42.63 | mg | 70 | 59.9 | 17.37 | −19.6 | 2.05 | −18.7 | 18.52 | −2.61 | −7.34 | 2.13 | 0.2798 |
| | PLACEBO | | | 96 | 63.3 | 19.13 | −17.0 | 1.59 | −17.5 | 17.99 | | | | |
| WEEK 12 | AVP-786 | 28 | mg | 52 | 62.5 | 21.12 | −17.7 | 2.00 | −14.3 | 16.77 | −0.92 | −5.82 | 3.99 | 0.7129 |
| | AVP-786 | 42.63 | mg | 64 | 57.3 | 17.69 | −22.2 | 2.05 | −21.6 | 18.18 | −5.41 | −10.1 | −0.68 | 0.0252 |

| | | | | | | | |
|---|---|---|---|---|---|---|---|
| PLACEBO | 92 | 63.8 | 17.85 | −16.8 | 1.58 | −17.8 | 17.37 |

TABLE 68X
SUMMARY OF MEAN CHANGE FROM BASELINE TO WEEK 12 IN CMAI TOTAL SCORE - MMRM, UN IN PATIENTS
BASELINE 12 <= MMSE_BASE <=20 (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | N | | | MEAN | SD | LSMEAN¹ | SE¹ | MEAN | SD | DIFF¹ | LOWER¹ | UPPER¹ | VALUE¹ |
| BASELINE | AVP-786 | 28 | mg | 43 | 75.9 | 21.88 | | | | | | | | |
| | AVP-786 | 42.63 | mg | 52 | 76.5 | 18.75 | | | | | | | | |
| | PLACEBO | | | 76 | 73.0 | 20.93 | | | | | | | | |
| WEEK 1 | AVP-786 | 28 | mg | 40 | 67.2 | 18.91 | −9.27 | 1.98 | −9.1 | 18.13 | −5.64 | −10.5 | −0.81 | 0.0224 |
| | AVP-786 | 42.63 | mg | 50 | 68.9 | 20.15 | −8.01 | 2.12 | −7.6 | 12.46 | −4.38 | −9.06 | 0.30 | 0.0661 |
| | PLACEBO | | | 74 | 74.4 | 21.37 | −3.63 | 1.56 | −3.8 | 10.90 | | | | |
| WEEK 2 | AVP-786 | 28 | mg | 35 | 64.8 | 19.23 | −12.7 | 2.21 | −13.0 | 16.61 | −4.56 | −9.99 | 0.88 | 0.0996 |
| | AVP-786 | 42.63 | mg | 49 | 67.0 | 19.18 | −10.5 | 2.27 | −10.2 | 14.68 | −2.36 | −7.53 | 2.81 | 0.3676 |
| | PLACEBO | | | 64 | 70.4 | 21.44 | −8.18 | 1.74 | −9.6 | 13.18 | | | | |
| WEEK 3 | AVP-786 | 28 | mg | 42 | 59.9 | 18.83 | −16.7 | 2.07 | −16.3 | 15.70 | −6.91 | −12.0 | −1.80 | 0.0083 |
| | AVP-786 | 42.63 | mg | 51 | 63.5 | 18.27 | −13.0 | 2.21 | −12.7 | 13.20 | −3.27 | −8.23 | 1.68 | 0.1939 |
| | PLACEBO | | | 72 | 68.5 | 21.14 | −9.74 | 1.65 | −10.0 | 14.99 | | | | |
| WEEK 6 | AVP-786 | 28 | mg | 39 | 59.1 | 19.86 | −15.9 | 2.39 | −13.6 | 15.92 | −4.92 | −10.8 | 0.92 | 0.0981 |
| | AVP-786 | 42.63 | mg | 50 | 62.7 | 20.74 | −14.0 | 2.41 | −13.3 | 15.13 | −3.06 | −8.61 | 2.49 | 0.2783 |
| | PLACEBO | | | 72 | 67.3 | 21.06 | −10.9 | 1.84 | −11.2 | 14.83 | | | | |
| WEEK 9 | AVP-786 | 28 | mg | 35 | 55.6 | 19.03 | −18.8 | 2.42 | −16.1 | 16.19 | −3.45 | −9.37 | 2.48 | 0.2522 |
| | AVP-786 | 42.63 | mg | 51 | 60.1 | 17.93 | −16.8 | 2.41 | −15.9 | 16.19 | −1.45 | −7.02 | 4.12 | 0.6086 |
| | PLACEBO | | | 69 | 62.9 | 20.31 | −15.4 | 1.86 | −16.2 | 16.19 | | | | |
| WEEK 12 | AVP-786 | 28 | mg | 36 | 60.5 | 20.63 | −17.3 | 2.39 | −13.3 | 17.13 | −2.02 | −7.90 | 3.85 | 0.4977 |
| | AVP-786 | 42.63 | mg | 47 | 56.9 | 18.07 | −20.2 | 2.41 | −19.0 | 14.96 | −4.93 | −10.5 | 0.65 | 0.0830 |
| | PLACEBO | | | 64 | 63.3 | 18.79 | −15.2 | 1.86 | −16.3 | 16.13 | | | | |

TABLE 68Y
SUMMARY OF MEAN CHANGE FROM BASELINE TO WEEK 12 IN CMAI TOTAL SCORE - MMRM,
UN IN FEMALES (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 53 | 74.1 | 19.85 | | | | | | | | |
| | AVP-786 | 42.63 | mg | 60 | 77.1 | 19.15 | | | | | | | | |
| | PLACEBO | | | 91 | 80.7 | 21.96 | | | | | | | | |
| WEEK 1 | AVP-786 | 28 | mg | 52 | 68.3 | 20.93 | −6.91 | 1.65 | −5.8 | 10.41 | 0.56 | −3.27 | 4.39 | 0.7726 |
| | AVP-786 | 42.63 | mg | 57 | 70.2 | 19.63 | −7.81 | 1.82 | −6.9 | 12.95 | −0.34 | −4.14 | 3.47 | 0.8621 |
| | PLACEBO | | | 89 | 73.1 | 20.89 | −7.48 | 1.32 | −7.4 | 11.48 | | | | |
| WEEK 2 | AVP-786 | 28 | mg | 45 | 67.3 | 20.82 | −10.4 | 1.95 | −8.9 | 10.72 | −0.30 | −4.91 | 4.32 | 0.8988 |
| | AVP-786 | 42.63 | mg | 56 | 66.7 | 20.17 | −11.3 | 2.03 | −10.4 | 15.17 | −1.27 | −5.76 | 3.21 | 0.5765 |
| | PLACEBO | | | 80 | 71.5 | 21.79 | −10.1 | 1.54 | −10.3 | 13.91 | | | | |
| WEEK 3 | AVP-786 | 28 | mg | 52 | 62.5 | 21.51 | −13.5 | 2.15 | −11.9 | 13.68 | −1.30 | −6.47 | 3.87 | 0.6207 |
| | AVP-786 | 42.63 | mg | 58 | 64.8 | 20.11 | −12.7 | 2.24 | −11.6 | 14.46 | −0.44 | −5.50 | 4.62 | 0.8634 |
| | PLACEBO | | | 86 | 68.0 | 20.59 | −12.2 | 1.70 | −12.9 | 17.96 | | | | |
| WEEK 6 | AVP-786 | 28 | mg | 49 | 62.1 | 22.67 | −13.8 | 2.25 | −12.3 | 15.75 | 0.69 | −4.70 | 6.08 | 0.8015 |
| | AVP-786 | 42.63 | mg | 56 | 65.1 | 21.53 | −12.7 | 2.30 | −11.0 | 15.32 | 1.79 | −3.45 | 7.03 | 0.5025 |
| | PLACEBO | | | 86 | 66.8 | 20.43 | −14.5 | 1.75 | −15.1 | 17.57 | | | | |
| WEEK 9 | AVP-786 | 28 | mg | 47 | 59.2 | 21.02 | −16.5 | 2.24 | −14.4 | 16.88 | 1.35 | −4.02 | 6.71 | 0.6213 |
| | AVP-786 | 42.63 | mg | 57 | 60.9 | 19.75 | −17.5 | 2.27 | −16.1 | 15.54 | 0.40 | −4.78 | 5.58 | 0.8791 |
| | PLACEBO | | | 83 | 63.3 | 19.54 | −17.9 | 1.74 | −19.1 | 17.83 | | | | |
| WEEK 12 | AVP-786 | 28 | mg | 46 | 63.0 | 22.86 | −15.4 | 2.25 | −13.3 | 18.97 | 4.46 | −0.91 | 9.84 | 0.1030 |
| | AVP-786 | 42.63 | mg | 54 | 58.3 | 18.12 | −20.0 | 2.28 | −18.8 | 15.51 | −0.19 | −5.40 | 5.02 | 0.9430 |
| | PLACEBO | | | 90 | 61.4 | 19.08 | −19.8 | 1.76 | −21.7 | 16.86 | | | | |

TABLE 68Z
SUMMARY OF MEAN CHANGE FROM BASELINE TO WEEK 12 IN CMAI TOTAL SCORE - MMRM, UN IN
PATIENTS NOT TAKING SSRIS AT BASELINE (MITT POPULATION, BASELINE FACTOR 1 PATIENTS)

| | TREAT- | | | | | | | | | | | | | |
|---|---|---|---|---|---|---|---|---|---|---|---|---|---|---|
| | MENT | | | | | | CHANGE FROM BASELINE | | | | | 95% CI | | P- |
| VISIT | GROUP | | | N | MEAN | SD | LSMEAN¹ | SE¹ | MEAN | SD | DIFF¹ | LOWER¹ | UPPER¹ | VALUE¹ |
| BASELINE | AVP-786 | 28 | mg | 66 | 71.7 | 20.16 | | | | | | | | |
| | AVP-786 | 42.63 | mg | 75 | 76.1 | 19.86 | | | | | | | | |
| | PLACEBO | | | 109 | 75.6 | 20.36 | | | | | | | | |
| WEEK 1 | AVP-786 | 28 | mg | 66 | 63.8 | 18.97 | −8.71 | 1.48 | −7.9 | 16.17 | −4.15 | −7.80 | −0.51 | 0.0257 |
| | AVP-786 | 42.63 | mg | 72 | 69.9 | 18.82 | −6.33 | 1.66 | −6.5 | 11.21 | −1.77 | −5.35 | 1.81 | 0.3311 |
| | PLACEBO | | | 108 | 70.9 | 20.54 | −4.56 | 1.25 | −4.4 | 10.66 | | | | |
| WEEK 2 | AVP-786 | 28 | mg | 54 | 60.6 | 19.30 | −12.7 | 1.60 | −11.8 | 14.93 | −6.76 | −10.6 | −2.88 | 0.0007 |
| | AVP-786 | 42.63 | mg | 71 | 67.0 | 19.46 | −9.54 | 1.70 | −9.7 | 13.24 | −3.59 | −7.30 | 0.13 | 0.0584 |
| | PLACEBO | | | 98 | 69.8 | 19.99 | −5.95 | 1.31 | −6.9 | 11.15 | | | | |
| WEEK 3 | AVP-786 | 28 | mg | 63 | 58.8 | 19.09 | −14.3 | 1.72 | −13.3 | 15.60 | −5.98 | −10.2 | −1.75 | 0.0058 |
| | AVP-786 | 42.63 | mg | 74 | 65.2 | 19.92 | −10.4 | 1.83 | −10.7 | 12.50 | −2.09 | −6.18 | 2.01 | 0.3165 |
| | PLACEBO | | | 103 | 67.7 | 20.13 | −8.34 | 1.42 | −8.3 | 15.68 | | | | |
| WEEK 6 | AVP-786 | 28 | mg | 59 | 59.5 | 18.96 | −12.7 | 1.93 | −10.8 | 14.92 | −2.77 | −7.49 | 1.95 | 0.2488 |
| | AVP-786 | 42.63 | mg | 73 | 63.3 | 21.67 | −12.4 | 1.96 | −12.5 | 14.79 | −2.50 | −6.98 | 1.98 | 0.2732 |
| | PLACEBO | | | 103 | 65.9 | 20.30 | −9.94 | 1.54 | −10.2 | 15.95 | | | | |
| WEEK 9 | AVP-786 | 28 | mg | 50 | 56.6 | 18.39 | −16.4 | 1.90 | −14.6 | 14.75 | −2.53 | −7.14 | 2.08 | 0.2804 |
| | AVP-786 | 42.63 | mg | 72 | 60.5 | 20.05 | −15.4 | 1.90 | −15.7 | 15.37 | −1.55 | −5.87 | 2.77 | 0.4795 |
| | PLACEBO | | | 100 | 62.1 | 18.82 | −13.9 | 1.49 | −14.1 | 15.78 | | | | |
| WEEK 12 | AVP-786 | 28 | mg | 50 | 57.2 | 18.54 | −17.2 | 2.00 | −14.6 | 16.34 | −1.52 | −6.38 | 3.34 | 0.5381 |
| | AVP-786 | 42.63 | mg | 69 | 56.8 | 18.57 | −19.1 | 1.97 | −19.5 | 16.15 | −3.43 | −7.96 | 1.11 | 0.1381 |
| | PLACEBO | | | 96 | 60.2 | 18.23 | −15.7 | 1.56 | −16.3 | 17.36 | | | | |

### 4.3.1.3. Additional CMAI Analyses

The CMAI Total score was also analyzed for the percentage of patients meeting response criteria, with response criteria defined as a 30% or 50% improvement in the CMAI Total score compare to Baseline, using the GEE model; Fisher's exact test was used if GEE model did not converge. There were no significant group differences at Week 12 using either the 30% (p=0.943 and p=0.845 for AVP-786-28 and AVP-786-42.63, respectively) or the 50% threshold (p=0.789 and p=0.251, respectively).

It was also determined that AVP 786-42.63 provided a significant treatment difference, as determined by a reduction in the CMAI total score, in patients that had an CMAI aggressive behavior score of greater than 15 prior to administration of therapeutically effective amounts of d6-DM and quinidine sulfate.

### 4.3.1.4. Secondary Efficacy Endpoints

4.3.1.4.1. mADCS-CGIC-Agitation Score

Since the primary analysis in the FWE control failed, the results of the key secondary endpoint analysis, mADCS-CGIC-Agitation score, are presented descriptively with nominal p-values. The difference between the average treatment effect of AVP 786 28 and AVP 786 42.63 versus placebo was not significant in the overall combined MMRM using the average of the 2 active treatment groups (p=0.841; Table 69), and the comparison to placebo at Week 12 was not significant for either the AVP 786 28 (p=0.484) or AVP 786 42.63 (p=0.704) groups. The treatment differences (CI) versus placebo at Week 12 were 0.1 (−0.2 to 0.4) for AVP 786-28 and −0.1 (−0.3 to 0.2) for AVP-78642.63 (Table 69).

TABLE 69
mADCS-CGIC-Agitation Score at Week 12: MMRM - Observed Data (miTT Population)

| Parameter/Results | Placebo | | AVP-786-28 | | AVP-786-42.63 | |
|---|---|---|---|---|---|---|
| Week 12 Score Relative to Baseline | 190 | | 118 | | 141 | |
| 1 = Marked Improvement, n (%) | 19 | (10.0) | 6 | (5.1) | 19 | (13.5) |
| 2 = Moderate Improvement, n (%) | 52 | (27.4) | 35 | (29.7) | 36 | (25.5) |
| 3 = Minimal Improvement, n (%) | 64 | (33.7) | 43 | (36.4) | 56 | (39.7) |
| 4 = No change, n (%) | 37 | (19.5) | 18 | (15.3) | 17 | (12.1) |
| 5 = Minimal Worsening, n (%) | 14 | (7.4) | 10 | (8.5) | 8 | (5.7) |
| 6 = Moderate Worsening, n (%) | 3 | (1.6) | 5 | (4.2) | 5 | (3.5) |
| 7 = Marked Worsening, n (%) | 1 | (0.5) | 1 | (0.8) | 0 | |
| Week 12 Score Relative to Baseline: N, Mean (SD) | 190, 2.9 | (1.18) | 118, 3.1 | (1.23) | 141, 2.8 | (1.20) |
| Score Relative to Baseline: LS Mean (SE) ¹ | 3.0 | (0.14) | 3.1 | (0.16) | 2.9 | (0.16) |

| | | | |
|---|---|---|---|
| Treat Diff vs. Placebo: p-value (Dif, 95% CD¹ | | 0.484 (0.1, −0.2 to 0.4) | 0.704 (−0.1, −0.3 to 0.2) |
| MMRM: N | 204 | 141 | 159 |

| | |
|---|---|
| Treat Diff vs. Placebo: p-value (Dif, 95% CI) ¹ | 0.841 (0.0, −0.2 to 0.2) |
| | (average AVP-786-28 and 42.63) |

CI = confidence interval;
Dif/Diff = difference;
LS = least squares;
mADCS-CGIC-Agitation = modified Alzheimer's Disease Cooperative Study-Clinical Global Impression of Change scale for Agitation;
mITT = modified intent-to-treat;
MMRM = mixed model repeated measures;
NPI AA = Neuropsychiatric Inventory Agitation/Aggression domain
Note:
mADCS-CGIC-Agitation Total Score ranges from 1 to 7 with lower scores indicating improvement.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

### 4.3.1.4.2. CGIS—Agitation Score

The mean changes from Baseline to Week 12 in CGIS-Agitation score were similar for all groups (Table 70). The treatment difference at Week 12 was not significant in the combined comparison (p=0.203) or for the comparison of AVP 786 28 (p=0.468) or AVP 786 42.63 (p=0.158) versus placebo. The treatment differences (CI) versus placebo at Week 12 were 0.1 (0.3 to 0.1) for both AVP 786-28 and AVP-786-42.63.

TABLE 70
CGIS-Agitation Score at Week 12: Change from Baseline MMRM - Observed Data (mITT Population)

| Parameter/Results | Placebo | | AVP-786-28 | | AVP-786-42.63 | |
|---|---|---|---|---|---|---|
| Baseline: N | 210 | | 150 | | 159 | |
| 0 = Not assessed, n (%) | 0 | | 0 | | 0 | |
| 1 = Normal, not at all ill, n (%) | 0 | | 0 | | 0 | |
| 2 = Borderline ill, n (%), | 0 | | 0 | | 0 | |
| 3 = Mildly ill, n (%) | 0 | | 0 | | 0 | |
| 4 = Moderately ill, n (%) | 131 | (62.4%) | 94 | (62.7%) | 99 | (62.3%) |
| 5 = Markedly ill, n (%) | 70 | (33.3%) | 52 | (34.7%) | 56 | (35.2%) |
| 6 = Severely ill, n (%) | 9 | (4.3%) | 3 | (2.0%) | 4 | (2.5%) |
| 7 = Among the most extremely ill patient, n (%) | 0 | | 1 | (0.7%) | 0 | |
| Baseline: N, Mean (SD) | 210, 4.4 | (0.58) | 150, 4.4 | (0.57) | 159, 4.4 | (0.54) |
| Week 12: N | 190 | | 118 | | 141 | |
| 0 = Not assessed, n (%) | 0 | | 0 | | 0 | |
| 1 = Normal, not at all ill, n (%) | 3 | (1.6%) | 3 | (2.5%) | 3 | (2.1%) |
| 2 = Borderline ill, n (%) | 8 | (4.2%) | 13 | (11.0%) | 15 | (10.6%) |
| 3 = Mildly ill, n (%) | 57 | (30.0%) | 29 | (24.6%) | 41 | (29.1%) |
| 4 = Moderately ill, n (%) | 96 | (50.5%) | 47 | (39.8%) | 63 | (44.7%) |
| 5 = Markedly ill, n (%) | 21 | (11.1%) | 23 | (19.5%) | 18 | (12.8%) |
| 6 = Severely ill, n (%) | 5 | (2.6%) | 2 | (1.7%) | 1 | (0.7%) |
| 7 = Among the most extremely ill patient, n (%) | 0 | | 1 | (0.8%) | 0 | |
| Week 12 Change from Baseline: N, Mean (SD) | 190, −0.7 | (0.85) | 118, −0.7 | (0.92) | 141, −0.8 | (0.93) |
| Change from Baseline: LS Mean (SE) ¹ | −0.9 | (0.10) | −1.0 | (0.12) | −1.0 | (0.12) |

| | | | |
|---|---|---|---|
| Treat Diff vs. Placebo: p-value (Dif, 95% CI) ¹ | | 0.468 (−0.1, −0.3 to 0.1) | 0.158 (−0.1, −0.3 to 0.1) |
| MMRM: N | 204 | 143 | 159 |

| | |
|---|---|
| Week 12 Score Relative to Baseline: | Average AVP-786-28 and 42.63 |
| Treat Diff vs. Placebo: p-value (Dif, 95% CI) ¹ | 0.203 (−0.1, −0.3 to 0.1) |

CGIS-Agitation = Clinical Global Impression of Severity of Illness scale for Agitation;
CI = confidence interval;
Dif/Diff = difference;
LS = least squares;
mITT = modified intent-to-treat;
MMRM = mixed model repeated measures;
NPI AA = Neuropsychiatric Inventory Agitation/Aggression domain
Note:
CGIS-Agitation scores range from 1 to 7 with higher scores indicating worsening condition.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

### 4.3.1.4.3. NPI Total Score and NPI Domain Scores

The NPI parameters pre-identified as endpoints Agitation/Aggression (domain score and Caregiver Distress score), Aberrant Motor Behavior (Domain score), and Irritability/Lability (domain score) and the NPI Total score are summarized in Table 71, and these results are discussed individually below. Other NPI endpoints are addressed below.

TABLE 71
NPI Domain and Total Scores at Week 12: Change from Baseline MMRM - Observed Data (mITT Population)

| Parameter/Results | Placebo | | AVP-786-28 | | AVP-786-42.63 | |
|---|---|---|---|---|---|---|
| Agitation/Aggression (Domain Score) | | | | | | |
| Baseline: N, Mean (SD) | 210, 6.8 | (2.41) | 150, 6.5 | (2.37) | 159, 7.0 | (2.04) |
| Week 12 Change from Baseline: N, Mean (SD) | 192, −2.5 | (3.17) | 123, −2.3 | (3.32) | 148, −3.2 | (3.32) |
| Change from Baseline: LS Mean (SE) ¹ | −2.7 | (0.29) | −3.0 | (0.34) | −3.3 | (0.33) |
| Treat Diff vs Placebo: p-value (Dif, 95% CI) ¹ | | | 0.416 (−0.3, −0.9 to 0.4) | | 0.066 (−0.6, −1.3 to 0.0) | |
| Agitation/Aggression (Caregiver Distress Score) | | | | | | |
| Baseline: N, Mean (SD) | 210, 3.1 | (0.97) | 150, 2.9 | (1.16) | 159, 2.9 | (1.07) |
| Week 12 Change from Baseline: N, Mean (SD) | 189, −0.8 | (1.44) | 121, −0.8 | (1.61) | 144, −0.8 | (1.47) |
| Change from Baseline: LS Mean (SE) ¹ | −0.8 | (0.13) | −1.0 | (0.15) | −1.0 | (0.14) |
| Treat Diff vs Placebo: p-value (Dif, 95% CI) ¹ | | | 0.249 (−0.2, −0.5 to 0.1) | | 0.199 (−0.2, −0.5 to 0.1) | |
| Aberrant Motor Behavior (Domain Score) | | | | | | |
| Baseline: N, Mean (SD) | 210, 4.9 | (3.72) | 150, 4.8 | (3.89) | 159, 5.2 | (3.71) |
| Week 12 Change from Baseline: N, Mean (SD) | 192, −1.2 | (3.75) | 123, −1.2 | (3.60) | 148, −1.8 | (3.57) |
| Change from Baseline: LS Mean (SE) ¹ | −1.4 | (0,33) | −1.4 | (0.38) | −1.7 | (0.38) |
| Treat Diff vs Placebo: p-value (Dif, 95% CI) ¹ | | | 0.975 (0.0, −0.7 to 0.7) | | 0.334 (−0.3, −1.0 to 0.3) | |
| Irritability/Lability (Domain Score) | | | | | | |
| Baseline: N, Mean (SD) | 210, 4.9 | (3.65) | 150, 3.9 | (3.46) | 159, 4.5 | (3.45) |
| Week 12 Change from Baseline: N, Mean (SD) | 1.92, −1.8 | (3.50) | 123, −1.2 | (3.55) | 148, −2.2 | (3.29) |
| Change from Baseline: LS Mean (SE) ¹ | −1.5 | (0.31) | −1.6 | (0.35) | −2.3 | (0.35) |
| Treat Diff vs Placebo: p-value (Dif, 95% CI) ¹ | | | 0.888 (−0.0, −0.7 to 0.6) | | 0.019 (−0.7, −1.3 to −0.1) | |
| NPI Total Score | | | | | | |
| Baseline: N, Mean (SD) | 210, 37.9 | (19.94) | 150, 34.2 | (19.06) | 159, 39.2 | (19.08) |
| Week 12 Orange from Baseline: N, Mean (SD) | 192, −12.3 | (17.06) | 123, −9.5 | (18.41) | 148, −16.9 | (18,05) |
| Change from Baseline: LS Mean (SE) ¹ | −12.5 | (1.73) | −11.9 | (1.95) | −16.1 | (1.95) |

| | | |
|---|---|---|
| Treat Diff vs Placebo: p-value (Dif, 95% CI) ¹ | 0.756 (0.6, −2.9 to 4.0) | 0.038 (−3.6, −7.0 to −0.2) |

CI = confidence interval;
Dif/Diff = difference;
LS = least squares;
mITT = modified intent-to-treat;
MMRM = mixed model repeated measures;
NPI = Neuropsychiatric Inventory;
SD = standard deviation;
SE = standard error
Note:
Agitation/Aggression Domain Score ranges from 0 to 12. Agitation/Aggression Domain - Caregiver Distress score ranges from 0 to 5. Aberrant Motor Behavior Domain score ranges from 0 to 12. Irritability/Lability Domain score ranges from 0 to 12. NPI Total score ranges from 0 to 144. For all scores, higher scores indicate greater worsening condition.
¹ MMRMs include fixed effects of treatment, visit, treatment-by-visit, Baseline, Baseline-by-visit, Baseline NPI = Agitation/Aggression (≤6 vs >6), risk assessment for falls (normal/mild vs moderate/severe), Baseline concomitant use of antipsychotic medications (yes vs no), and Cohort (Cohort 1 vs Cohort 2) if applicable. Unstructured variance-covariance was used.

The changes from Baseline in the NPI Agitation/Aggression Domain scores at various time points are shown in the Table 71a below:

TABLE 71a
NPI Agitation/Aggression Domain Score: Change from Baseline MMRM - Observed Data (mITT Population)

| Parameter/Results | Placebo | | AVP-786 28 mg | | AVP-786 42.63 mg | |
|---|---|---|---|---|---|---|
| Baseline: N, Mean (SD) | 210, 6.8 | (2.41) | 150, 6.5 | (2.37) | 159, 7.0 | (2.04) |
| Week 1 Change from Baseline: N, Mean (SD) | 206, −0.8 | (2.45) | 142, −1.0 | (2.34) | 153, −1.0 | (2.25) |
| Change from Baseline: LS Mean (SE) ¹ | −1.1 | (0.25) | −1.4 | (0.27) | −1.2 | (0.28) |
| Treat Diff vs. Placebo: p-value (Dif, 95% CI) ¹ | | | 0.190 (−0.3, −0.8 to 0.2) | | 0.779 (−0.1.−0.5 to 0.4) | |
| Week 2 Change from Baseline: N, Mean (SD) | 185, −1.4 | (2.62) | 131, −1.4 | (2.89) | 149, −1.6 | (2.39) |
| Change from Baseline: LS Mean (SE) ¹ | −1.6 | (0.26) | −1.9 | (0.28) | −1.7 | (0.29) |
| Treat Diff vs. Placebo: p-value (Dif, 95% CI) ¹ | | | 0.179 (−0.3, −0.8 to 0.2) | | 0.514 (−0.2, −0.7 to 0.3) | |
| Week 3 Change from Baseline: N, Mean (SD) | 199, −1.3 | (2.93) | 145, −1.8 | (2.97) | 155, −1.9 | (2.62) |
| Change from Baseline: LS Mean (SE) ¹ | −1.6 | (0.27) | −2.4 | (0.29) | −2.1 | (0.30) |
| Treat Diff vs. Placebo: p-value (Dif, 95% CI) ¹ | | | 0.009 (−0.7, −1.3 to −0.2) | | 0.116 (−0.4, −1.0 to 0.1) | |
| Week 6 Change from Baseline: N, Mean (SD) | 202, −1.8 | (3.13) | 137, −2.0 | (3.56) | 153, −2.5 | (2.91) |
| Change from Baseline: LS Mean (SE) ¹ | −2.0 | (0.28) | −2.6 | (0.32) | −2.6 | (0.32) |
| Treat Diff vs. Placebo: p-value (Dif, 95% CI) ¹ | | | 0.079 (−0.6, −1.2 to 0.1) | | 0.074 (−0.6, −1.2 to 0.1) | |
| Week 9 Change from Baseline: N, Mean (SD) | 195, −2.2 | (3.02) | 120, −2.2 | (3.29) | 151, −2.8 | (3.06) |
| Change from Baseline: LS Mean (SE) ¹ | −2.5 | (0.28) | −2.8 | (0.32) | −2.9 | (0.31) |
| Treat Diff vs. Placebo: p-value (Dif, 95% CI) ¹ | | | 0.310 (−0.3, −0.9 to 0.3) | | 0.135 (−0.4, −1.0 to 0.1) | |
| Week 12 Change from Baseline: N, Mean (SD) | 192, −2.5 | (3.17) | 123, −2.3 | (3.32) | 148, −3.2 | (3.32) |
| Change from Baseline: LS Mean (SE) ¹ | −2.7 | (0.29) | −3.0 | (0.34) | −3.3 | (0.33) |

| | | |
|---|---|---|
| Treat Diff vs. Placebo: p-value (Dif, 95% CI) ¹ | 0.416 (−0.3, −0.9 to 0.4) | 0.066 (−0.6, −1.3 to 0.0) |

Note:
Agitation/Aggression Domain Score ranges from 0 to 12 with higher scores indicating worsening condition.
¹ MMRMs include fixed effects of treatment, visit, treatment-by-visit, baseline, baseline-by-visit, baseline NPI AA (≤6 vs. >6), risk assessment for falls (normal/mild vs. moderate/severe), baseline concomitant use of antipsychotic medications (yes vs. no), and Cohort (Cohort 1 vs. Cohort 2) if applicable. Unstructured variance-covariance was used.

### NPI—Agitation/Aggression Domain Score and Caregiver Distress Score

There were no significant differences between placebo and AVP 786 28 or AVP 786 42.63 in the NPI—Agitation/Aggression domain at Week 12 (p=0.416 and 0.066 for placebo vs AVP 786 28 and AVP 786 42.63, respectively) or the NPI—Agitation/Aggression Caregiver Distress score (p=0.249 and 0.199).

The rate of NPI—Agitation/Aggression responders was summarized with responders defined as patients with a 30% improvement from Baseline and separately with responders defined as patients with a 50% improvement from Baseline. There were no significant between-group differences in response rate using either response criteria.

### NPI—Aberrant Motor Behavior Domain Score

There were no significant differences between placebo and AVP-786-28 or AVP-786-42.63 at Week 12 in the NPI—Aberrant Motor Behavior Domain score (p=0.975 and 0.334 for placebo vs AVP-786-28 and AVP-786-42.63, respectively.

### NPI—Irritability/Lability Domain Score

Patients treated with AVP-786-42.63 had a significantly greater improvement in NPI—Irritability/Lability domain score compared with the placebo group (significant at the nominal level, p=0.019), with a treatment difference (CI) of −0.7 (−1.3 to −0.1). There were no significant differences between placebo and AVP 786 28 at Week 12 in the NPI Total score (p=0.756).

### NPI Total Score

Patients treated with AVP-786-42.63 had a significantly greater improvement in NPI Total score compared with the placebo group (significant at the nominal level, p=0.038), with a treatment difference (CI) of 3.6 (−7.0 to −0.2). There were no significant differences between placebo and AVP 786 28 at Week 12 in the NPI Total score (p=0.756.

### 4.3.1.4.4. NPI: Other Domains

Significant treatment differences (at the nominal level) in favor of AVP-786 versus placebo at Week 12 include:
NPI—NPI4A score, AVP-786-42.63 vs placebo: p=0.006
NPI—Irritability/Lability domain score, AVP-786-42.63 vs placebo: p=0.019

### 4.3.1.4.5. ADCS-CGIC-Overall Score

The treatment difference at Week 12 for the ADCS-CGIC-Overall score was not significant for comparison of AVP 786 28 (p=0.400) or AVP 786 42.63 (p=0.566) versus placebo, as presented in Table 72.

TABLE 72
ADCS-CGIC-Overall Score at Week 12: MMRM - Observed Data (mITT Population)

| Paramcter/Results | Placebo | | AVP-786-28 | | AVP-786-42.63 | |
|---|---|---|---|---|---|---|
| Week 12 Score Relative to Baseline: N | 188 | | 118 | | 141 | |
| 1 = Marked Improvement, n (%) | 9 | (4.8) | 7 | (5.9) | 9 | (6.4) |
| 2 = Moderate Improvement, n (%) | 49 | (26.1) | 26 | (22.0) | 38 | (27.0) |
| 3 = Minimal Improvement, n (%) | 60 | (31.9) | 33 | (28.0) | 56 | (39.7) |
| 4 = No change, n (%) | 40 | (21.3) | 26 | (22.0) | 20 | (14.2) |
| 5 = Minimal Worsening, n (%) | 25 | (13.3) | 21 | (17.8) | 9 | (6.4) |
| 6 = Moderate Worsening, n (%) | 4 | (2.1) | 4 | (3.4) | 9 | (6.4) |
| 7 = Marked Worsening, n (%) | 1 | (0.5) | 1 | (0.8) | 0 | |
| Week 12 Score Relative to Baseline: N, Mean (SD) | 188, 3.2 | (1.20) | 118, 3.4 | (1.31) | 141, 3.1 | (1.23) |
| Score Relative to Baseline: LS Mean (SE) ¹ | 3.4 | (0.14) | 3.5 | (0.16) | 3.3 | (0.16) |

| | | |
|---|---|---|
| Treat Diff vs. Placebo: p-value (Dif, 95% CI) ¹ | 0.400 (0.1, −0.2 to 0.4) | 0.566 (−0.1, −0.3 to 0.2) |

ADCS-CGIC-Overall = Alzheimer's Disease Cooperative Study-Clinical Global Impression of Change for Overall Clinical Status;
CI = confidence interval;
Dif/Diff = difference;
LS = least squares;
mITT = modified intent-to-treat;
MMRM = mixed model repeated measures;
NPI AA = Neuropsychiatric Inventory Agitation/Aggression domain;
SD = standard deviation;
SE = standard error
Note:
ADCS-CGIC-Overall ranges from 1 to 7 with higher scores indicating worsening condition.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) applicable. Unstructured variance-covariance was used.

### 4.3.1.4.6. PGIC Score

The treatment difference at Week 12 for the PGIC score was not significant for comparison of AVP 786 28 (p=0.751) or AVP 786 42.63 (p=0.320) versus placebo, as presented in Table 73. A PGIC response summary (with patients reporting much improved or very much improved counted as responders). The GEE model did not indicate a significant difference from placebo for either AVP 786 28 (p=0.492) or AVP 786 42.63 (p=0.123).

TABLE 73
PGIC Score at Week 12: MMRM - Observed Data (mITT Population)

| Parameter/Results | Placebo | | AVP-786-28 | | AVP-786-42.63 | |
|---|---|---|---|---|---|---|
| Week 12 Score Relative to Baseline: N | 191 | | 118 | | 141 | |
| 1 = Very much improved, n (%) | 7 | (3.7) | 2 | (1.7) | 7 | (5.0) |
| 2 = Much improved, n (%) | 52 | (27.2) | 37 | (31.4) | 49 | (34.8) |
| 3 = Minimally improved, n (%) | 74 | (38.7) | 39 | (33.1) | 54 | (383) |
| 4 = No change, n (%) | 42 | (22.0) | 26 | (22.0) | 17 | (12.1) |
| 5 = Minimally worse, n (%) | 14 | (73) | 12 | (10.2) | 13 | (9.2) |
| 6 = Much worse, n (%) | 2 | (1.0) | 2 | (1.7) | 1 | (0.7) |
| 7 = Very much worse, n (%) | 0 | | 0 | | 0 | |
| Week 12 Score Relative to Baseline: N, Mean (SD) | 191, 3.1 | (1.01) | 118, 3.1 | (1.07) | 141, 2.9 | (1.05) |
| Score Relative to Baseline: LS Mean (SE) ¹ | 3.0 | (0.13) | 3.1 | (0.14) | 2.9 | (0.14) |

| | | |
|---|---|---|
| Treat Diff vs. Placebo: p-value (Dif, 95% CI) ¹ | 0.751 (0.0, −0.2 to 0.3) | 0.320 (−0.1, −0.3 to 0.1) |

CI = confidence interval; Dif/Diff = difference; LS = least squares; mITT = modified intent-to-treat; MMRM = mixed model repeated measures; NPI AA = Neuropsychiatric Inventory Agitation/Aggression domain; PGIC = Patient Global Impression of Change; SD = standard deviation; SE = standard error.
Note:
PGIC score ranges from 1 to 7 with higher scores indicating worsening condition.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

### 4.3.1.4.7. Zarit Burden Interview

The treatment difference at Week 12 was not significant for the comparison of AVP-786-28 (p=0.934) or AVP-786-42.63 (p=0.342) versus placebo. The treatment difference with reference to ZBI Total Score is presented in Table 74.

TABLE 74
ZBI Total Score at Week 12: Change from Baseline MMRM - Observed Data (mITT Population)

| Parameter/Results | Placebo | | AVP-786-28 | | AVP-786-42.63 | |
|---|---|---|---|---|---|---|
| Baseline; N, Mean (SD) | 210, 28.93 | (15.965) | 150, 30.21 | (15.983) | 159, 29.33 | (15.371) |
| Week 12 Change from Baseline: N, Mean (SD) | 190, −1.77 | (9.275) | 118, −1.03 | (12.195) | 141, −0.23 | (11.769) |
| Change from Baseline: LS Mean (SE) ¹ | −1.4 | (1.31) | −1.5 | (1.47) | −0.3 | (1.47) |

| | | |
|---|---|---|
| Treat Diff vs. Placebo: p-value (Dif, 95% CI) ¹ | 0.934 (−0.1, −2.4 to 2.2) | 0.342 (1.1, −1.2 to 3.4) |

CI = confidence interval;
Dif/Diff = difference;
LS = least squares;
mITT = modified intent-to-treat;
MMRM = mixed model repeated measures;
NPI AA = Neuropsychiatric Inventory Agitation/Aggression domain;
SD = standard deviation;
SE = standard error;
ZBI = Zarit Burden Interview
Note:
ZBI Total score ranges from 0 to 88 with higher scores indicating greater caregiver burden.
¹ MMRMs include fixed effects of treatment, visit, treatment-by-visit, Baseline, Baseine-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) applicable. Unstructured variance-covariance was used.

### 4.3.1.4.8. DEMQOL Total Score

The treatment difference at Week 12 was not significant for the comparison of AVP 786 28 (p=0.782) or AVP 786 42.63 (p=0.991) versus placebo. The treatment difference with reference to DEMQOL Total Score is presented in Table 75.

TABLE 75
DEMQOL Total Score at Week 12: Change from Baseline MMRM - Observed Data (mITT Population)

| Parameter/Results | Placebo | | AVP-786-28 | | AVP-786 -42.63 | |
|---|---|---|---|---|---|---|
| Baseline: N, Mean (SD) | 170, 83.1 | (11.87) | 120, 83.5 | (11.96) | 132, 82.5 | (11.96) |
| Week 12 Change from Baseline: N, Mean (SD) | 153, 2.9 | (11.10) | 96, 3.8 | (8.10) | 117, 3.2 | (8.74) |
| Change from Baseline: LS Mean (SE) ¹ | 4.5 | (1.44) | 4.8 | (1.57) | 4.5 | (1.54) |

| | | |
|---|---|---|
| Treat Diff vs. Placebo: p-value (Dif, 95% CI) ¹ | 0.782 (0.3, −1.8 to 2.4) | 0.991 (−0.0, −2.1 to 2.0) |

CI = confidence interval;
DEMQOL = Dementia Quality of Life;
Dif/Diff = difference;
LS = least squares;
mITT = modified intent-to-treat;
MMRM = mixed model repeated measures;
NPI AA = Neuropsychiatric Inventory Agitation/Aggression domain;
SD = standard deviation;
SE = standard error
Note:
DEMQOL Total score ranges from 28 to 112 with higher scores indicating greater quality of life.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

Using the DEMQOL-Proxy score, the improvement from Baseline to Week 12 in QOL was greatest with placebo, and the comparison of AVP 786 42.63 vs placebo was significant (p=0.006); as presented in Table 76, indicating decreased QOL for the AVP-786-42.63 group.

TABLE 76
DEMQOL-Proxy Score at Week 12: Change from Baseline MMRM - Observed Data (mITT Population)

| Parameter/Results | Placebo | | AVP-786-28 | | AVP-786-42.63 | |
|---|---|---|---|---|---|---|
| Baseline: N, Mean (SD) | 209, 89.5 | (13.15) | 150, 89.3 | (13.03) | 158, 88.2 | (12.68) |
| Week 12 Change from Baseline: N, Mean (SD) | 189, 5.4 | (11.68) | 118, 3.4 | (10.74) | 140, 2.4 | (11.08) |
| Change from Baseline: LS Mean (SE) ¹ | 7.8 | (1.25) | 6.3 | (1.41) | 4.6 | (1.40) |

| | | |
|---|---|---|
| Treat Diff vs. Placebo: p-value (Dif, 95% CI) ¹ | 0.193 (−1.5, −3.9 to 0.8) | 0.006 (−3.2, −5.5 to −0.9) |

CI = confidence interval;
DEMQOL = Dementia Quality of Life;
Dif/Diff = difference;
LS = least squares;
mITT = modified intent-to-treat;
MMRM = mixed model repeated measures;
NPI AA = Neuropsychiatric Inventory Agitation/Aggression domain;
SD = standard deviation;
SE = standard error
Note:
The DEMQOL-Proxy scale ranges from 31 to 124 with higher scores indicating greater quality of life.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

### 4.3.1.4.9. CSDD Score

The treatment difference in CSDD score at Week 12 was not significant for the comparison of AVP 786 42.63 (p=0.911) versus placebo, as presented in Table 77. The treatment difference favored placebo in the comparison of AVP-786-28 and placebo at Week 12 (p=0.038; Table 77).

TABLE 77
CSDD Score at Week 12: Change from Baseline MMRM - Observed Data (mITT Population)

| Parameter/Results | Placebo | | AVP-786-28 | | AVP-786-42.63 | |
|---|---|---|---|---|---|---|
| Baseline: N, Mean (SD) | 210, 6.6 | (2.13) | 150, 6.5 | (2.14) | 159, 6.8 | (1.94) |
| Week 12 Change from Baseline: N, Mean (SD) | 189, −1.1 | (2.84) | 118, −0.5 | (2.90) | 141, −1.5 | (2.57) |
| Change from Baseline: LS Mean (SE) ¹ | −0.9 | (0.32) | −0.3 | (0.36) | −0.9 | (0.36) |

| | | |
|---|---|---|
| Treat Diff vs. Placebo: p-value (Dif, 95% CI) ¹ | 0.038 (0.6, 0.0 to 1.2) | 0.911 (−0.0, −0.6 to 0.5) |

CI = confidence interval;
CSDD = Cornell Scale for Depression in Dementia;
Dif/Diff = difference;
LS = least squares;
mITT = modified intent-to-treat;
MMRM = mixed model repeated measures;
NPI AA = Neuropsychiatric Inventory Agitation/Aggression domain;
SD = standard deviation:
SE = standard error
Note:
CSDD score ranges from 0 to 38 with higher scores indicating evidence of depression.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

### 4.3.1.4.10. GMHR

The GMHR was only performed at Baseline and Week 12. At Baseline, the proportions of patients with ratings of “good” (no patients responded “excellent to very good” in any group at Baseline) were 35.7%, 29.3%, and 38.0% on placebo, AVP-786-28 and AVP-786-42.63, respectively. At Week 12, the proportion reporting “excellent to very good” and “good” combined were 62.9%, 62.0%, and 58.2%, respectively.

### 4.3.1.4.11. ADAS-cog

The difference at Week 12 was not significant for the comparison of AVP-786-28 (p=0.236) or AVP-786-42.63 (p=0.684) versus placebo (Table 78). There was no worsening of cognition for the AVP-786 treatment groups compared to placebo.

TABLE 78
ADAS-cog at Week 12: Change from Baseline ANCOVA - LOCF Data (mITT Population)

| Parameter/Results | Placebo | | AVP-786-28 | | AVP-786-42.63 | |
|---|---|---|---|---|---|---|
| Baseline: N, Mean (SD) | 163, 29.0 | (10.58) | 113, 27.1 | (9.62) | 113, 27.4 | (9.30) |
| Week 12 Change from Baseline: N, Mean (SD) | 143, −2.0 | (5.67) | 91, −1.0 | (7.36) | 97, −1.5 | (6.38) |
| Change from Baseline: LS Mean (SE) ¹ | 1.0 | (1.02) | 2.0 | (1.12) | 1.3 | (1.12) |

| | | |
|---|---|---|
| Treat Diff vs. Placebo: p-value (Dif, 95% CI) ¹ | 0.236 (1.0, −0.6 to 2.6) | 0.684 (0.3, −1.3 to 1.9) |

ADAS-cog = Alzheimer's Disease Assessment Scale-cognitive subscale;
ANCOVA = analysis of covariance;
CI = confidence interval;
Dif/Diff = difference;
OCF = last observation carried forward;
LS = least squares;
mITT = modified intent-to-treat;
NPI AA = Neuropsychiatric Inventory Agitation/Aggression domain;
SD = standard deviation;
SE = standard error
Note:
ADAS-cog scores range from 0 to 70 with higher scores indicating greater cognitive impairment.
¹ MMRMs include fixed effects of treatment, visit, treatment-by-visit, Baseline, Baseline-by-visit, Baseline NPI AA (≤6 vs. >6), risk assessment for falls (normal/mild vs. moderate/severe), Baseline concomitant use of antipsychotic medications (yes vs. no), and Cohort (Cohort 1 vs. Cohort 2) if applicable. Unstructured variance-covariance was used.

Caregiver demographics, care giver work status, and patient living accommodations were similar among the groups at Baseline, and few caregivers in any group reported using healthcare resources at Baseline. The amount of time caregivers spent at various caregiver tasks was similar in all groups at Baseline and did not change notably from Baseline to Week 12. Few hospital or emergency room visits were reported in the 30 days preceding Baseline, Week 6, or Week 12 in any group. Most patients in all groups (approximately 90% to 95% in each treatment group at each time point) visited a healthcare profession in the 30 days preceding Baseline, Week 6, and Week 12. Most of these visits were to “other” healthcare professionals. There were no notable differences between groups in healthcare utilization.

### 4.3.1.5. Use of an “Efficacy Subset” of Patients

The primary analysis was performed on the mITT Population, defined as all randomized patients with at least 1 postbaseline efficacy assessment.

### 4.3.1.6. Examination of Subgroups

CMAI data were summarized by concomitant medication and Baseline status subgroup. Few clinically meaningful subgroup differences were observed.

Patients who were not receiving Baseline psychotropic concomitant medications that are major CYP2D6 substrates appeared to have slightly higher Baseline CMAI scores than patients receiving such medications, but there was no evidence of a meaningful difference in response between placebo and active treatment in either group.

For patients who were not receiving Baseline beta blocker concomitant medications that are major CYP2D6 substrates, the LS mean difference in CMAI Total score (AVP-786-42.63 minus placebo) was −18.4 (p=0.001) at Week 12; the AVP-786-28 group was intermediate and was not significantly different from placebo. Note, however, that the number of patients in these groups were small (10, 9, and 15 patients on placebo, AVP-786-28, and AVP-786-42.63, respectively), and the placebo group had notably higher Baseline scores (90.6, 57.9, and 72.4). More patients were included in the subgroup that were receiving Baseline beta blocker concomitant medications that are major CYP2D6 substrates groups, and the means were similar at Baseline and at Week 12.

The CMAI subscales F1—Aggressive Behavior, F2—Physically Nonaggressive Behavior, and F3—Verbally Agitated Behavior were reanalyzed in the subgroup of patients who were agitated (as defined in the CMAI manual and elsewhere herein) at Baseline. The differences from placebo in changes from Baseline to Week 12 were not significant for the AVP-786-28 and AVP-786-42.63 combined group or either active group individually.

An additional subgroup analysis was performed comparing the proportions of patients who were agitated vs not agitated, as defined separately for each CMAI subscale. Shift tables from Baseline to end of treatment for agitated vs not agitated status (as defined in the CMAI manual and elsewhere herein) are presented for CMAI Factor 1, Aggressive Behavior, CMAI Factor 2—Physically Non-aggressive Behavior, and CMAI Factor 3—Verbally Agitated Behavior. On Factor 1, the proportions of patients with shifts from agitated at Baseline to non-agitated at end of treatment were 65/166 (number of patients who shifted from agitated at Baseline to non-agitated at end of treatment/number of patients agitated at Baseline) (39.2%), 40/99 (40.4%), and 48/104 (46.2%) for placebo, AVP-786-28, and AVP-786-42.63, respectively). On Factor 2, the proportions were 33/187 (17.6%), 37/131 (28.2%), and 33/140 (23.6%). On Factor 3, the proportions were 33/192 (17.2%), 36/135 (26.7%), and 29/149 (19.5%).

### 4.3.2. Drug Dose, Drug Concentration, and Relationships to Response

See Section 5.4 and Section 5.5.

### 4.3.3. By-Patient Displays

No additional by-patient displays of efficacy data were prepared.

### 4.3.4. Efficacy Conclusions

Primary Efficacy Endpoint (CMAI Total Score):
The primary test for the predefined FWE control (closed testing) procedure was not passed, so results from the individual treatment groups are presented descriptively. Patients treated with AVP-786-28 and AVP-786-42.63 showed declines (improvement) from Baseline in CMAI Total score at Week 12; however, the changes from Baseline were similar to those in the placebo group and were not significantly different from placebo. The treatment differences (CI) versus placebo at Week 12 were 0.4 (−2.7 to 3.5; p=0.789) for AVP-786-28 and −2.0 (−5.0 to 1.0; p=0.200) for AVP-786-42.63.
Sensitivity Analyses: Results from the sensitivity analysis of CMAI Total score supported the primary analysis.

Key Secondary Efficacy Endpoints (mADCS-CGIC-Agitation Score and CGIS-Agitation Score):
For mADCS-CGIC-Agitation score, the difference between the average treatment effect of AVP-786-28 and AVP-786-42.63 versus placebo was not significant in the overall combined MMRM (p=0.841); the comparison to placebo at Week 12 was not significant for either the AVP-786-28 (p=0.484) or AVP-786-42.63 (p=0.704) groups. The treatment differences (CI) versus placebo at Week 12 were 0.1 (−0.2 to 0.4) for AVP-786-28 and −0.1 (−0.3 to 0.2) for AVP-786-42.63.
For CGIS-Agitation score, the treatment difference at Week 12 was not significant in the combined comparison (p=0.203) or for the comparison of AVP-786-28 (p=0.468) or AVP-786-42.63 (p=0.158) versus placebo. The treatment differences (CI) versus placebo at Week 12 were −0.1 (−0.3 to 0.1) for both AVP-786-28 and AVP-786-42.63.

Other Secondary Efficacy Endpoints:

For the NPI—Irritability/Lability domain score, the decrease (improvement) from Baseline to Week 12 was significantly greater (at the nominal level, p=0.019) for AVP-786-42.63 (treatment difference [CI] of −0.7 [−1.3 to −0.1]). The difference was not significant for AVP-786-28.

For the NPI Total score, the decrease (improvement) from Baseline to Week 12 was significantly greater (at the nominal level, p=0.038) for AVP-786-42.63 (treatment difference [Cl] of 3.6 [−7.0 to −0.2]). The difference was not significant for AVP-786-28.

### 4.4. Pharmacokinetic and Pharmacodynamic Results

### 4.4.1. d6-DM, Q, and Metabolite Plasma Concentrations

At Day 43 (Visit 4; Week 6) and Day 85 (Visit 6; Week 12), patients had a blood sample collected between 0 and 3 hours after the morning dose of study medication for analysis of plasma levels of d6-DM, d6-DM metabolites, and Q. The time when the patient was administered the dose of study medication and the time of the blood draw were recorded. Plasma samples were separated by centrifugation and frozen at −20° C. until assayed at the analytical unit.

Plasma concentration data for d6-DM and its metabolites d3-dextrorphan (d3-DX), d3-3-methoxymorphinan (d3-3-MM), and Q are summarized in Table 79 by metabolite, CYP2D6 metabolizer group, treatment group, and visit. There were no notable differences between Week 6 and Week 12 for any analyte at any dose level for all patients combined or in any metabolizer group. In general, d6-DM and d3-3-MM concentrations were highest in the poor and intermediate metabolizer groups and lowest in the ultra-rapid metabolizer group. Exposure to d3-DX was lowest in the poor metabolizer group and highest in the extensive and ultra-rapid metabolizer groups. Plasma concentrations as shown by mean and median values were generally higher for d6-DM and d3-DX at the higher d6-DM dose, and Q values were similar at both d6-DM doses.

TABLE 79
Plasma Concentrations of d6-Dextromethorphan, d3-Dextrorphan, d3-3-Methoxymorphinan,
and Quinidine by Metabolizer Group, Treatment Group, and Week

| | | Poor | | Intermediate | | Extensive | | Ultra-Rapid | | | |
|---|---|---|---|---|---|---|---|---|---|---|---|
| | | Metabolizer | | Metabolizer | | Metabolizer | | Metabolizer | | All | |
| Analyte (unit) | | (PM) | | (IM) | | (EM) | | (UM) | | Patients | |
| Treatment Visit | Statistic | (N = 10) | | (N = 98) | | (N = 114) | | (N = 10) | | (N = 232) | |
| AVP-786 28 mg | | d6-Dextromethorphan (μg/L) | | | | | | | | | |
| (N = 151) | | | | | | | | | | | |
| Week 6 | n | 3 | | 41 | | 53 | | 5 | | 102 | |
| | Mean (SD) | 148.60 | (123.600) | 80.96 | (58.165) | 46.95 | (52.062) | 43.73 | (72.689) | 63.45 | (61.230) |
| | Median | 149.00 | | 83.70 | | 28.60 | | 15.00 | | 47.95 | |
| | Min, Max | 24.8, 272.0 | | 0.0, 193.0 | | 0.0, 258.0 | | 0.0, 172.0 | | 0.0, 272.0 | |
| Week 12 | n | 3 | | 41 | | 52 | | 5 | | 101 | |
| | Mean (SD) | 53.23 | (78.187) | 71.10 | (58.021) | 43.25 | (40.828) | 55.24 | (76.287) | 55.44 | (52.247) |
| | Median | 16.70 | | 73.90 | | 35.20 | | 8.39 | | 43.90 | |
| | Min, Max | 0.0, 143.0 | | 0.0, 191.0 | | 0.0, 146.0 | | 0.0, 170.0 | | 0.0, 191.0 | |
| AVP-786 42.63 mg | | | | | | | | | | | |
| (N = 160) | | | | | | | | | | | |
| Week 6 | n | 5 | | 51 | | 57 | | 5 | | 118 | |
| | Mean (SD) | 131.07 | (167.968) | 66.10 | (83.983) | 54.82 | (53.701) | 2.45 | (4.564) | 60.70 | (75.802) |
| | Median | 65.10 | | 18.10 | | 40.20 | | 0.00 | | 29.35 | |
| | Min, Max | 0.0, 398.0 | | 0.0, 335.0 | | 0.0, 226.0 | | 0.0, 10.5 | | 0.0, 398.0 | |
| Week 12 | n | 7 | | 47 | | 52 | | 5 | | 111 | |
| | Mean (SD) | 145.49 | (130.161) | 67.30 | (78.150) | 64.41 | (67.222) | 2.17 | (3.031) | 67.94 | (78.479) |
| | Median | 138.00 | | 28.10 | | 43.60 | | 0.00 | | 34.00 | |
| | Min, Max | 0.0, 314.0 | | 0.0, 274.0 | | 0.0, 255.0 | | 0.0, 6.3 | | 0.0, 314.0 | |
| AVP-786 28 mg | | | | | | | | | | | |
| (N = 151) | | | | | | | | | | | |
| Week 6 | n | 3 | | 41 | | 53 | | 5 | | 102 | |
| | Mean (SD) | 191.10 | (158.642) | 49.36 | (45.796) | 28.46 | (46.774) | 20.74 | (37.628) | 41.26 | (57.549) |
| | Median | 246.00 | | 43.70 | | 11.30 | | 1.94 | | 22.05 | |
| | Min, Max | 12.3, 315.0 | | 0.0, 194.0 | | 0.0, 283.0 | | 0.0, 87.3 | | 0.0, 315.0 | |
| Week 12 | n | 3 | | 41 | | 52 | | 5 | | 101 | |
| | Mean (SD) | 113.67 | (115.544) | 43.78 | (46.082) | 22.19 | (31.677) | 20.46 | (34.278) | 33.59 | (44.568) |
| | Median | 110.00 | | 27.90 | | 10.12 | | 3.02 | | 15.60 | |
| | Min, Max | 0.0, 231.0 | | 0.0, 162.0 | | 0.0, 148.0 | | 0.0, 80.1 | | 0.0, 231.0 | |
| AVP-786 42.63 mg | | | | | | | | | | | |
| (N = 160) | | | | | | | | | | | |
| Week 6 | n | 6 | | 52 | | 57 | | 5 | | 120 | |
| | Mean (SD) | 69.81 | (89.286) | 35.92 | (54.233) | 24.91 | (29.923) | 0.47 | (0.769) | 30.91 | (46.480) |
| | Median | 23.65 | | 4.74 | | 10.60 | | 0.00 | | 6.28 | |
| | Min, Max | 0.0, 190.0 | | 0.0, 219.0 | | 0.0, 130.0 | | 0.0, 1.8 | | 0.0, 219.0 | |
| Week 12 | n | 7 | | 47 | | 52 | | 5 | | 111 | |
| | Mean (SD) | 85.73 | (92.869) | 33.17 | (48.134) | 26.90 | (31.909) | 0.46 | (0.644) | 32.08 | (46.402) |
| | Median | 40.30 | | 2.36 | | 11.10 | | 0.00 | | 8.31 | |
| | Min, Max | 0.0, 248.0 | | 0.0, 201.0 | | 0.0, 102.0 | | 0.0, 1.3 | | 0.0, 248.0 | |
| AVP-786 28 mg | | | | | | | | | | | |
| (N = 151) | | | | | | | | | | | |
| Week 6 | n | 3 | | 41 | | 53 | | 5 | | 102 | |
| | Mean (SD) | 28.80 | (22.848) | 121.13 | (71.028) | 163.37 | (116.505) | 272.20 | (21.2.721) | 147.77 | (111.317) |
| | Median | 34.00 | | 128.00 | | 144.00 | | 289.00 | | 134.50 | |
| | Min, Max | 3.8, 48.6 | | 0.0, 247.0 | | 0.0, 423.0 | | 0.0, 562.0 | | 0.0, 562.0 | |
| Week 12 | n | 3 | | 41 | | 49 | | 5 | | 98 | |
| | Mean (SD) | 12.90 | (19.717) | 112.61 | (74.364) | 164.81 | (112.718) | 189.00 | (181.805) | 139.56 | (105.782) |
| | Median | 3.11 | | 111.00 | | 148.00 | | 240.00 | | 135.50 | |
| | Min, Max | 0.0, 35.6 | | 0.0, 270.0 | | 0.0, 433.0 | | 0.0, 401.0 | | 0.0, 433.0 | |
| AVP-786 42.63 mg | | | | | | | | | | | |
| (N = 160) | | | | | | | | | | | |
| Week6 | n | 6 | | 51 | | 55 | | 5 | | 117 | |
| | Mean (SD) | 15.29 | (16.804) | 120.24 | (116.419) | 229.75 | (164.534) | 71.06 | (148.342) | 164.24 | (153.696) |
| | Median | 9.98 | | 107.00 | | 265.00 | | 0.00 | | 128.00 | |
| | Min, Max | 0.0, 40.2 | | 0.0, 579.0 | | 0.0, 702.0 | | 0.0, 336.0 | | 0.0, 702.0 | |
| Week 12 | n | 7 | | 46 | | 51 | | 5 | | 109 | |
| | Mean (SD) | 13.54 | (11.104) | 112.86 | (106.276) | 194.82 | (154.668) | 73.78 | (138.291) | 143.04 | (139.496) |
| | Median | 16.20 | | 110.00 | | 195.00 | | 0.00 | | 134.00 | |
| | Min, Max | 0.0, 27.4 | | 0.0, 415.0 | | 0.0, 623.0 | | 0.0, 318.0 | | 0.0, 623.0 | |
| AVP-786 28 mg | | | | | | | | | | | |
| (N = 151) | | | | | | | | | | | |
| Week 6 | n | 3 | | 41 | | 53 | | 5 | | 102 | |
| | Mean (SD) | 14.78 | (10.379) | 24.48 | (15.868) | 21.67 | (16.484) | 23.20 | (20.365) | 22.67 | (16.183) |
| | Median | 16.00 | | 26.80 | | 18.90 | | 20.30 | | 22.25 | |
| | Min, Max | 3.9, 24.5 | | 0.0, 67.9 | | 0.0, 72.6 | | 0.0, 55.2 | | 0.0, 72.6 | |
| Week 12 | n | 3 | | 41 | | 52 | | 5 | | 101 | |
| | Mean (SD) | 5.80 | (10.046) | 22.32 | (18.250) | 26.92 | (22.088) | 18.16 | (19.222) | 23.99 | (20.398) |
| | Median | 0.00 | | 21.90 | | 24.40 | | 18.30 | | 21.90 | |
| | Min, Max | 0.0, 17.4 | | 0.0, 87.6 | | 0.0, 120.0 | | 0.0, 45.3 | | 0.0, 120.0 | |
| AVP-786 42.63 mg | | | | | | | | | | | |
| (N = 160) | | | | | | | | | | | |
| Week 6 | n | 6 | | 52 | | 57 | | 5 | | 120 | |
| | Mean (SD) | 19.70 | (17.887) | 18.74 | (16.036) | 19.02 | (1.3.043) | 3.44 | (7.692) | 18.28 | (14.677) |
| | Median | 22.25 | | 16.40 | | 19.60 | | 0.00 | | 17.80 | |
| | Min, Max | 0.0, 39.7 | | 0.0, 55.3 | | 0.0, 48.1 | | 0.0, 17.2 | | 0.0, 55.3 | |
| Week 12 | n | 7 | | 47 | | 52 | | 5 | | 111 | |
| | Mean (SD) | 21.80 | (17.212) | 16.65 | (16.391) | 21.85 | (18.245) | 2.68 | (5.993) | 18.78 | (17.404) |

| | | | | | |
|---|---|---|---|---|---|
| Median | 14.90 | 15.00 | 21.15 | 0.00 | 18.50 |
| Min, Max | 0.0, 47.5 | 0.0, 59.9 | 0.0, 72.3 | 0.0, 13.4 | 0.0, 72.3 |

Max = maximum;
Min = minimum;
SD = standard deviation
Note:
Patients who were not assigned a metabolizer group are excluded. Values that are below the limit of quantification (BLQ) are summarized as 0. Week 12 includes early termination visits.

### 4.5. Pharmacokinetic Summary and Discussion

There did not appear to be any significant changes in d6-DM, d3-DX, d3-3-MM, and Q plasma concentrations from Week 6 to Week 12 in all patients combined. Exposures to d6-DM and d3-DX increased with the increase in d6-DM dose in AVP-786.

### 5. Safety Evaluation

### 5.1. Extent of Exposure

The mean (SD) duration of exposure was similar across treatment groups: 82.4 (15.5), 76.1 (21.6), and 82.2 (15.7) days for patients who received placebo, AVP-786-28, and AVP-786-42.63, respectively (Table 80).

TABLE 80
Duration of Exposure (Safety Population)

| | Placebo | AVP-786-28 | AVP-786-42.63 |
|---|---|---|---|
| | (N = 210) | (N = 151) | (N = 160) |
| Duration of Exposure (days) | 210 | 151 | 160 |
| Mean (SD) | 82.4 (15.5) | 76.1 (21.6) | 82.2 (15.7) |
| Median | 85.0 | 85.0 | 85.0 |
| Min, Max | 5, 135 | 1, 105 | 1, 106 |
| Number of Patient-Days | 17, 31.2 | 11, 496 | 13, 157 |
| Number of Patient-Years | 47.40 | 31.47 | 36.02 |

SD = standard deviation
Note:
Denominators are the number of patients who had exposure data. Number of patient-years = summation over all patients' exposure in days divided by 365.25.

### 5.2. Adverse Events

TEAEs were collected at each visit after the Screening Visit and until 30 days after the last dose of study drug. TEAEs were defined as those adverse events that began or worsened on or after the first dose date and before the last dose date+30 days.

### 5.2.1. Brief Summary of Adverse Events

Of the 521 patients in the Safety Population, 241 patients (46.3%) reported a total of 639 TEAEs (Table 81). The incidence of TEAEs for patients in the AVP-786-28 and AVP-786-42.63 group was 48.3% and 46.3%, respectively, compared with 44.8% for the placebo group. Patients treated with AVP-786 had a higher incidence of TEAEs considered related to study drug by the Investigator compared with placebo (6.2% placebo, 12.6% AVP-786-28, and 15.0% AVP-786-42.63). The incidence of non-serious TEAEs was similar across treatment groups.

Overall, the incidences of SAEs (6.3%), discontinuations due to TEAEs (3.5%), and deaths (0.6%) were low in this elderly population. Patients treated with AVP-786-28 had a higher incidence of SAEs compared with the placebo group (9.9% vs 4.8%, respectively); however, the incidence was similar between the AVP-786-42.63 and placebo groups (5.0% vs 4.8%, respectively). Patients treated with AVP-786-28 also had a higher incidence of discontinuations due to TEAEs compared with placebo (6.6% vs 1.0%, respectively). Few patients had drug-related SAEs or discontinuations due to TEAEs, and none of the deaths were considered related to study drug by the Investigator.

TABLE 81
Overview of Treatment-emergent Adverse Events (Safety Population)

| | Placebo | | AVP-786-28 | | AVP-786-42.63 | | All Patients | |
|---|---|---|---|---|---|---|---|---|
| Parameter | (N = 210) | | (N = 151) | | (N = 160) | | (N = 521) | |
| Total number of AEs | 276 | | 216 | | 193 | | 685 | |
| Total number of TEAEs | 259 | | 198 | | 182 | | 639 | |
| Patients with at least one n (%) | | | | | | | | |
| TEAE | 94 | (44.8) | 73 | (48.3) | 74 | (46.3) | 241 | (46.3) |
| Drug-related TEAE | 13 | (6.2) | 19 | (12.6) | 24 | (15.0) | 56 | (10.7) |
| Non-serious TEAE | 92 | (43.8) | 67 | (44.4) | 70 | (43.8) | 229 | (44.0) |
| Serious TEAE | 10 | (4.8) | 15 | (9.9) | 8 | (5.0) | 33 | (6.3) |
| Drug-related serious TEAE | 0 | | 1 | (0.7) | 0 | | 1 | (0.2) |
| Patients discontinued treatment n (%) | | | | | | | | |
| Due to TEAE | 2 | (1.0) | 10 | (6.6) | 6 | (3.8) | 18 | (3.5) |
| Due to drag-related TEAE | 1 | (0.5) | 1 | (0.7) | 4 | (2.5) | 6 | (1.2) |
| Due to serious TEAE | 1 | (0.5) | 7 | (4.6) | 2 | (1.3) | 10 | (1.9) |

| Due to drug-related serious TEAE | 0 | 0 | | 0 | 0 | |
|---|---|---|---|---|---|---|
| Deaths n (%) | | | | | | |
| Patients who died | 0 | 3 | (2.0) | 0 | 3 | (0.6) |
| Patients who died due to TEAE | 0 | 3 | (2.0) | 0 | 3 | (0.6) |

| | | | | |
|---|---|---|---|---|
| Patients who died due to drug-related TEAE | 0 | 0 | 0 | 0 |

AE = adverse event;
TEAE = treatment-emergent adverse event
Note:
A treatment-emergent adverse event (TEAE) is defined as an AE where: first dose date ≤ AE start date ≤ last dose date + 30.
A drug-related event is defined as an event assessed as Possibly Related or Related by the Investigator or where Relationship records are missing.

### 5.2.2. Analysis of Adverse Events

### 5.2.2.1. Adverse Events of Greatest Incidence

The SOCs with the highest incidence of TEAEs (≥10% patients in any treatment group) included Infections and Infestations (18.1%, 17.2%, and 10.0% in the placebo, AVP-786-28, and AVP-786-42.63 groups, respectively), Nervous System Disorders (8.1%, 13.9%, and 11.3%, respectively), Gastrointestinal Disorders (11.0%, 9.9%, and 10.0%, respectively), Injury, Poisoning and Procedural Complications (5.7%, 12.6%, and 7.5%, respectively), and Psychiatric Disorders (11.0%, 4.0%, and 7.5%, respectively, Table 82).

By PT, of the most frequently reported TEAEs (≥2% of patients in any treatment group; Table 82), those that occurred in a higher percentage of patients in any active treatment group compared to placebo were fall (4.3%, 10.6%, and 6.3% patients in the placebo, AVP-786-28, and AVP-786-42.63 groups, respectively), urinary tract infection (5.2%, 7.3%, and 2.5% patients respectively), somnolence (1.0%, 2.6%, and 6.9% patients, respectively), dizziness (1.9%, 2.0%, and 4.4% patients, respectively), hot flush (0, 2.0%, and 0 patients, respectively), and syncope (0, 2.0%, and 0 patients, respectively).

Patients who received placebo had the highest incidence of agitation (5.7% placebo, 1.3% AVP-786-28, and 3.1% AVP-42.63).

TABLE 82
Summary of Treatment-emergent Adverse Events Occurring in ≥2% of Patients in
Any Treatment Group by System Organ Class and Preferred Term (Safety Population)

| | Placebo | | AVP-786-28 | | AVP-786-42.63 | | AH Patients | |
|---|---|---|---|---|---|---|---|---|
| System Organ Class (SOC) | (N = 210) | | (N = 151) | | (N = 160) | | (N = 521) | |
| Preferred Term (PT) | n (%) | | n (%) | | n (%) | | n (%) | |
| Patients with at least one TEAE | 94 | (44.8) | 73 | (48.3) | 74 | (46.3) | 241 | (46.3) |
| Gastrointestinal disorders | 23 | (11.0) | 15 | (9.9) | 16 | (10.0) | 54 | (10.4) |
| Diarrhoea | 14 | (6.7) | 5 | (3.3) | 5 | (3.1) | 24 | (4.6) |
| Nausea | 2 | (1.0) | 3 | (2.0) | 4 | (2.5) | 9 | (1.7) |
| Vomiting | 4 | (1.9) | 4 | (2.6) | 3 | (1.9) | 11 | (2.1) |
| Constipation | 2 | (1.0) | 4 | (2.6) | 2 | (1.3) | 8 | (1.5) |
| Infections and infestations | 38 | (18.1) | 26 | (17.2) | 16 | (10.0) | 80 | (15.4) |
| Nasopharyngitis | 4 | (1.9) | 2 | (1.3) | 6 | (3.8) | 12 | (2.3) |
| Urinary tract infection | 11 | (5.2) | 11 | (7.3) | 4 | (2.5) | 26 | (5.0) |
| Upper respiratory tract infection | 7 | (3.3) | 5 | (3.3) | 2 | (1.3) | 14 | (2.7) |
| Bronchitis | 2 | (1.0) | 3 | (2.0) | 1 | (0.6) | 6 | (1.2) |
| Pneumonia | 1 | (0.5) | 3 | (2.0) | 1 | (0.6) | 5 | (1.0) |
| Injury, poisoning and procedural | 12 | (5.7) | 19 | (12.6) | 12 | (7.5) | 43 | (8.3) |
| complications | | | | | | | | |
| Fall | 9 | (4.3) | 16 | (10.6) | 10 | (6.3) | 35 | (6.7) |
| Contusion | 5 | (2.4) | 3 | (2.0) | 5 | (3.1) | 13 | (2.5) |
| Skin abrasion | 1 | (0.5) | 4 | (2.6) | 2 | (1.3) | 7 | (1.3) |
| Musculoskeletal and connective tissue | 11 | (5.2) | 4 | (2.6) | 9 | (5.6) | 24 | (4.6) |
| disorders | | | | | | | | |
| Arthralgia | 5 | (2.4) | 2 | (1.3) | 1 | (0.6) | 8 | (1.5) |
| Nervous system disorders | 17 | (8.1) | 21 | (13.9) | 18 | (11.3) | 56 | (10.7) |
| Somnolence | 2 | (1.0) | 4 | (2.6) | 11 | (6.9) | 17 | (3.3) |
| Dizziness | 4 | (1.9) | 3 | (2.0) | 7 | (4.4) | 14 | (2.7) |
| Headache | 3 | (1.4) | 5 | (3.3) | 2 | (1.3) | 10 | (1.9) |
| Syncope | 0 | | 3 | (2.0) | 0 | | 3 | (0.6) |
| Psychiatric disorders | 23 | (11.0) | 6 | (4.0) | 12 | (7.5) | 41 | (7.9) |
| Agitation | 12 | (5.7) | 2 | (1.3) | 5 | (3.1) | 19 | (3.6) |
| Respiratory, thoracic and mediastinal | 15 | (7.1) | 7 | (4.6) | 3 | (1.9) | 25 | (4.8) |
| disorders | | | | | | | | |
| Cough | 6 | (2.9) | 1 | (0.7) | 2 | (1.3) | 9 | (1.7) |
| Vascular disorders | 6 | (2.9) | 10 | (6.6) | 6 | (3.8) | 22 | (4.2) |
| Hypertension | 3 | (1.4) | 2 | (1.3) | 4 | (2.5) | 9 | (1.7) |
| Hypotension | 1 | (0.5) | 3 | (2.0) | 1 | (0.6) | 5 | (1.0) |

| | | | | | | |
|---|---|---|---|---|---|---|
| Hot flush | 0 | 3 | (2.0) | 0 | 3 | (0.6) |

TEAE = treatment-emergent adverse event
Note:
Adverse events are coded using MedDRA version 18.1.
If a patient has more than one TEAE that codes to the same MedDRA category, the patient is counted only once in the MedDRA category.

### 5.2.2.2. Common Adverse Events by Time to Onset, Duration, and Recurrence

The common TEAEs that were reported at a higher incidence during active treatment were summarized by time to onset, duration, and recurrence. For these analyses, a common TEAE was defined as a TEAE with an incidence that was ≥3% in either AVP-786 group AND was ≥2 times the incidence of the placebo group. The only TEAE that met this definition was somnolence (1.0%, 2.6%, and 6.9% for placebo, AVP-786-28, and AVP-786-42.63, respectively); median time to onset of somnolence was 14.5, 9.5, and 2.0 days in the placebo, AVP-786-28, and AVP-786-42.63 groups, respectively.

The median duration of somnolence was similar across all treatment groups. The median duration of somnolence was 54 days (82.5% of study days), 59 days (69.4%), and 56 days (71.4%) in the placebo, AVP-786-28, and AVP-786-42.63 groups, respectively.

Two patients experienced a recurrence of a common TEAE; 2 patients in the AVP-786-42.63 group experienced recurrent somnolence.

### 5.2.2.3. Relationship of Adverse Events to Study Drug

Patients in the AVP-786-28 and AVP-786-42.63 groups had a higher incidence of drug-related TEAEs (12.6% and 15.0%, respectively) compared with the placebo group (6.2%; Table 83). By SOC, the most frequently reported drug-related TEAEs (≥3% patients in any treatment group) in the placebo, AVP-786-28, and AVP-786-42.63 groups were Nervous System Disorders (1.9%, 5.3%, and 8.1%, respectively) and Gastrointestinal Disorders (0.5%, 4.0%, and 4.4%, respectively).

The most frequently reported drug-related TEAEs (≥2% of patients in any treatment group) were somnolence (1.0%, 1.3%, and 5.6% patients in the placebo, AVP-786-28, and AVP-786-42.63 groups, respectively), dizziness (0, 0.7%, and 2.5% patients, respectively), headache (0.5%, 2.0%, and 0.6% patients, respectively), and fall (0, 2.0%, and 0 patients, respectively; Table 83).

TABLE 83
Summary of Drug-related Treatment-emergent Adverse Events Experienced by
≥2 Patients by System Organ Class and Preferred Term (Safety Population)

| | Placebo | | AVP-786-28 | | AVP-786-42.6 | | All Patients | |
|---|---|---|---|---|---|---|---|---|
| | (N = 210) | | (N = 151) | | (N = 160) | | (N = 521) | |
| Preferred Term (PT) | n (%) | | n (%) | | n (%) 3 | | n (%) | |
| Patients with at least one drug-related TEAE | 13 | (6.2) | 19 | (12.6) | 24 | (15.0) | 56 | (10.7) |
| Cardiac disorders | 3 | (1.4) | 0 | | 0 | | 3 | (0.6) |
| Sinus bradycardia | 2 | (1.0) | 0 | | 0 | | 2 | (0.4) |
| Ear and labyrinth disorders | 1 | (0.5) | 0 | | 1 | (0.6) | 2 | (0.4) |
| Vertigo | 1 | (0.5) | 0 | | 1 | (0.6) | 2 | (0.4) |
| Gastrointestinal disorders | 1 | (0.5) | 6 | (4.0) | 7 | (4.4) | 14 | (2.7) |
| Diarrhoea | 1 | (0.5) | 1 | (0.7) | 3 | (1.9) | 5 | (1.0) |
| Constipation | 0 | | 1 | (0.7) | 2 | (1.3) | 3 | (0.6) |
| Nausea | 1 | (0.5) | 2 | (1.3) | 1 | (0.6) | 4 | (0.8) |
| General disorders and administration site | 1 | (0.5) | 3 | (2.0) | 1 | (0.6) | 5 | (1.0) |
| conditions | | | | | | | | |
| Asthenia | 0 | | 1 | (0.7) | 1 | (0.6) | 2 | (0.4) |
| Gait disturbance | 0 | | 2 | (1.3) | 0 | | 2 | (0.4) |
| Fatigue | 1 | (0.5) | 1 | (0.7) | 0 | | 2 | (0.4) |
| Injury, poisoning and procedural | 0 | | 3 | (2.0) | 0 | | 3 | (0.6) |
| complications | | | | | | | | |
| Fall | 0 | | 3 | (2.0) | 0 | | 3 | (0.6) |
| Metabolism and nutrition disorders | 2 | (1.0) | 1 | (0.7) | 0 | | 3 | (0.6) |
| Decreased appetite | 2 | (1.0) | 0 | | 0 | | 2 | (0.4) |
| Nervous system disorders | 4 | (1.9) | 8 | (5.3) | 13 | (8.1) | 25 | (4.8) |
| Somnolence | 2 | (1.0) | 2 | (1.3) | 9 | (5.6) | 13 | (2.5) |
| Dizziness | 0 | | 1 | (0.7) | 4 | (2.5) | 5 | (1.0) |
| Headache | 1 | (0.5) | 3 | (2.0) | 1 | (0.6) | 5 | (1.0) |

TEAE = treatment-emergent adverse event
Note:
Drug-related is defined as an event assessed as Possibly Related or Related by the Investigator or where Relationship records are missing.
Adverse events are coded using MedDRA version 18.1.
If a patient has more than one TEAE that codes to the same MedDRA category, the patient is counted only once in the MedDRA category.

### 5.2.2.4. Adverse Events by Severity

Overall, the incidence of severe TEAEs was low during the study (4.8%). Patients in the AVP-786-28 group had a higher incidence of severe TEAEs compared with the placebo group, but the incidence in the AVP-786-42.63 group was lower than placebo (4.3%, 8.60%, and 1.9% for the placebo, AVP-786-28, and AVP-786-42.63 groups, respectively). Severe TEAEs reported by more than 1 patient were pneumonia (0, 1.3%, and 0 patients, respectively), fall (0.5%, 0.7%, and 0 patients, respectively), eosinophil count increased (1.0%, 0, and 0 patients, respectively), encephalopathy (0, 0.7%, and 0.6% patients, respectively), syncope (0, 1.3%, and 0 patients, respectively), respiratory failure (0.5%, 0.7%, and 0 patients, respectively), and hypotension (0, 1.3%, and 0 patients, respectively).

### 5.2.2.5. Analysis of Adverse Events by Baseline Use of Major CYP2D6 Substrate Beta Blocker Concomitant Medications

There were 41, 32, and 37 patients in the placebo, AVP-786-28, and AVP-786-42.63 groups, respectively, who at the Baseline visit were using beta blockers that were major CYP2D6 substrates. In general, the overall TEAE profile for these patients was consistent with that observed for patients who were not using beta blockers that were major CYP2D6 substrates (Table 84) and in the full Safety Population.

TABLE 84
Summary of Treatment-emergent Adverse Events Experienced by ≥2 Patients
Overall with Baseline Beta Blocker Concomitant Medications Use that
are Major CYP2D6 Substrates by Preferred Term (Safety Population)

| | Placebo | AVP-786-28 | AVP-786-42.63 |
|---|---|---|---|
| System Organ Class (SOC) | (N = 41) | (N = 32) | (N = 37) |
| Preferred Term (PT) | n (%) | n (%) | n (%) |
| Diarrhoea | 3 (7.3) | 1 (3.1) | 3 (8.1) |
| Bronchitis | 2 (4.9) | 2 (6.3) | 1 (2.7) |
| Fall | 0 | 2 (6.3) | 3 (8.1) |
| Somnolence | 2 (4,9) | 1 (3.1) | 3 (8.1) |
| Vomiting | 2 (4.9) | 1 (3.1) | 1 (2.7) |
| Nasopharyngitis | 1 (2.4) | 1 (3.1) | 2 (5.4) |
| Urinary tract infection | 2 (4.9) | 1 (3.1) | 1 (2.7) |
| Atrial fibrillation | 1 (2.4) | 2 (6.3) | 0 |
| Nausea | 1 (2.4) | 1 (3.1) | 1 (2.7) |
| Contusion | 0 | 1 (3.1) | 2 (5.4) |
| Chronic obstructive pulmonary disease | 1 (2.4) | 2 (6.3) | 0 |
| Influenza like illness | 0 | 0 | 2 (5.4) |
| Influenza | 0 | 2 (6.3) | 0 |
| Blood urea increased | 2 (4.9) | 0 | 0 |
| Back pain | 1 (2.4) | 0 | 1 (2.7) |
| Dizziness | 0 | 1 (3.1) | 1 (2.7) |
| Agitation | 2 (4.9) | 0 | 0 |
| Pulmonary mass | 0 | 1 (3.1) | 1 (2.7) |
| Dyspnoea | 1 (2.4) | 0 | 1 (2.7) |
| Hypotension | 0 | 2 (6.3) | 0 |

MedDRA = Medical Dictionary for Regulatory Activities;
TEAE = treatment-emergent adverse event
Note:
Beta blockers that are major CYP2D6 substrates include: Carvedilol, Metoprolol, Propranolol, Timolol. All other beta blockers will not be considered major CYP2D6 substrates.
Adverse events are coded using MedDRA version 18.1.
If a patient has more than one TEAE that codes to the same MedDRA category, the patient is counted only once in the MedDRA category.

### 5.2.2.6. Deaths

Three patients (0.6% overall) died during this study (Table 85). All 3 deaths occurred in patients randomized to AVP-786-28, and the causes of death were pneumonia, encephalopathy, and respiratory failure. None of the deaths were considered related to study drug by the Investigator.

TABLE 85
Deaths (Safety Population)

| | | Start Day/ | Duration of AE | | Treatment Given for TEAE/ |
|---|---|---|---|---|---|
| | MedDRA SOC | End Day/ | (% of Study | | Relationship/ |
| Age/Race/Ethnicity/Sex | Preferred Term | (Days to Onset) | Days) | | Action Taken/ |
| AVP-786-28 | | | | | |
| 74/White/Male | Infections and infestations | 83/85/(82) | 3 | (3.6) | NO/ |
| | Pneumonia | | | | NOT RELATED/ |
| | | | | | DRUG WITHDRAWN/ |
| 84/White/Male | Nervous system disorders | 43/51/(42) | 9 | (25.0) | NO/ |
| | Encephalopathy | | | | NOT RELATED/ |
| | | | | | DRUG WITHDRAWN/ |
| 86/White/Male | Respiratory, thoracic and | 80/80/(79) | 1 | (1.3) | YES/ |
| | mediastinal disorders | | | | NOT RELATED/ |
| | Respiratory failure | | | | NOT APPLICABLE/ |

MedDRA = Medical Dictionary for Regulatory Activities;
SOC = System Organ Class;
TEAE = treatment-emergent adverse event=

### 5.2.2.7. Other Serious Adverse Events

Overall, 6.3% of patients experienced SAEs during the study. Patients in the AVP-786-28 group had a higher incidence of SAEs compared with the placebo group (9.9% and 4.8%, respectively); however, the incidence of SAEs was similar between the AVP-786-42.63 and placebo groups (5.0% and 4.8%, respectively). Few SAEs were reported in more than 1 patient (Table 86).

Overall, the SOC with the highest incidence of SAEs was Infections and Infestations, with 13 patients (2.5%) experiencing SAEs (1.9%, 3.3%, and 2.5% in the placebo, AVP-786-28, and AVP-786-42.63 groups, respectively).

By PT, SAEs experienced by more than 1 patient in any treatment group were pneumonia (0.5%, 2.0%, and 0.6% in the placebo, AVP-786-28, and AVP-786-42.63 groups, respectively), fall (0.5%, 2.0%, and 0%, respectively), mental status changes (0%, 0.7%, and 1.3%, respectively), and syncope (0%, 2.0%, and 0%, respectively; Table 86).

One patient reported SAEs that were considered possibly drug-related by the Investigator. A patient in the AVP-786-28 group (Patient 224-004) experienced possibly drug-related SAEs of syncope and fall. The syncope was severe in intensity, and the fall was moderate, and both events resolved on the same day. The patient also experienced an SAE of normal pressure hydrocephalus on the same day; this event was considered unrelated to study drug and moderate in intensity, and it resolved 2 days later. The patient discontinued treatment because of the SAE of normal pressure hydrocephalus.

TABLE 86
Summary of Treatment-emergent Serious Adverse Events Experienced
by ≥2 Patients by Preferred Term (Safety Population)

| | Placebo | AVP-786-28 | AVP-786-42.63 | All Patients |
|---|---|---|---|---|
| | (N = 210) | (N = 151) | (N = 160) | (N = 521) |
| Preferred Term (PT) | n (%) | n (%) | n (%) | n (%) |
| Patients with at least one serious TEAE | 10 (4.8) | 15 (9.9) | 8 (5.0) | 33 (6.3) |
| Pneumonia | 1 (0.5) | 3 (2.0) | 1 (0.6) | 5 (1.0) |
| Fall | 1 (0.5) | 3 (2.0) | 0 | 4 (0.8) |
| Mental status changes | 0 | 1 (0.7) | 2 (1.3) | 3 (0.6) |
| Syncope | 0 | 3 (2.0) | 0 | 3 (0.6) |
| Bronchitis | 0 | 1 (0.7) | 1 (0.6) | 2 (0.4) |
| Encephalopathy | 0 | 1 (0.7) | 1 (0.6) | 2 (0.4) |
| Respiratory failure | 1 (0.5) | 1 (0.7) | 0 | 2 (0.4) |

MedDRA = Medical Dictionary for Regulatory Activities; TEAE = treatment-emergent adverse event.
Note:
Adverse events are coded using MedDRA version 18.1.
If a patient has more than one TEAE that codes to the same MedDRA category, the patient is counted only once in the MedDRA category.

### 5.2.2.8. Other Significant Adverse Events

### 5.2.2.8.1. Adverse Events Leading to Discontinuation of Treatment

Overall, 3.5% of patients discontinued treatment due to at least one TEAE. A higher percentage of patients in the AVP-786-28 (6.6%) and AVP-786-42.63 (3.8%) groups discontinued treatment due to TEAEs compared with the placebo group (1.0%; Table 87). No single TEAE led to discontinuation of more than 1 patient in any treatment group.

Drug-related TEAEs led to discontinuation of treatment for 1 (0.5%), 1 (0.7%), and 4 (2.5%) patients in the placebo, AVP-786-28, and AVP-786-42.63 groups, respectively.

TABLE 87
Summary of Treatment-emergent Adverse Events Leading to
Discontinuation by Preferred Term (Safety Population)

| | Placebo | AVP-786-28 | AVP-786-42.63 | All Patients |
|---|---|---|---|---|
| System Organ Class (SOC) | (N = 210) | (N = 151) | (N = 160) | (N = 521) |
| Preferred Term (PT) | n (%) | n (%) | n (%) | n (%) |
| Patients with at least one TEAE | 2 (1.0) | 10 (6.6) | 6 (3.8) | 18 (3.5) |
| leading to discontinuation | | | | |
| Leukopenia | 0 | 1 (0.7) | 0 | 1 (0.2) |
| Diarrhoea | 0 | 1 (0.7) | 0 | 1 (0.2) |
| Asthenia | 0 | 0 | 1 (0.6) | 1 (0.2) |
| Gait disturbance | 0 | 1 (0.7) | 0 | 1 (0.2) |
| Pneumonia | 0 | 1 (0.7) | 0 | 1 (0.2) |
| Cellulitis | 1 (0.5) | 0 | 0 | 1 (0.2) |
| Lumbar vertebral fracture | 0 | 0 | 1 (0.6) | 1 (0.2) |
| Fall | 0 | 1 (0.7) | 0 | 1 (0.2) |
| Hepatic enzyme increased | 0 | 0 | 1 (0.6) | 1 (0.2) |
| Pancreatic carcinoma metastatic | 0 | 1 (0.7) | 0 | 1 (0.2) |
| Dizziness | 0 | 0 | 1 (0.6) | 1 (0.2) |
| Toxic neuropathy | 0 | 0 | 1 (0.6) | 1 (0.2) |
| Encephalopathy | 0 | 1 (0.7) | 0 | 1 (0.2) |
| Normal pressure hydrocephalus | 0 | 1 (0.7) | 0 | 1 (0.2) |
| Agitation | 0 | 0 | 1 (0.6) | 1 (0.2) |
| Mental status changes | 0 | 1 (0.7) | 0 | 1 (0.2) |
| Disorientation | 1 (0.5) | 0 | 0 | 1 (0.2) |
| Respiratory failure | 0 | 1 (0.7) | 0 | 1 (0.2) |
| Hypotension | 0 | 1 (0.7) | 0 | 1 (0.2) |

MedDRA = Medical Dictionary for Regulatory Activities; TEAE = treatment-emergent adverse event.
Note:
Adverse events are coded using MedDRA version 18.1.
If a patient has more than one TEAE that codes to the same MedDRA category, the patient is counted only once in the MedDRA category.

### 5.2.2.8.2. Treatment-Emergent Adverse Events of Interest

### Fall

Fall was the most frequently reported TEAE across all treatment groups, with 6.7% patients overall reporting at least 1 TEAE of fall (Table 88). Patients in the AVP-786-28 group had the highest incidence of falls (10.6%) compared with patients in the placebo (4.3%) and AVP-786-42.63 (6.3%) groups. Overall, 4 patients experienced falls that were SAEs (1 [0.5%] placebo and 3 [2.0%] AVP-786-28), and only 1 patient (AVP-786-28) experienced a serious fall that was considered related to study drug. The majority of the falls were mild to moderate in severity. Two patients (1 [0.5%] placebo and 1 [0.7%] AVP-786-28) experienced severe falls. One patient experienced a fall that resulted in discontinuation from treatment.

TABLE 88
Summary of Treatment-emergent Adverse Events - Fall (Safety Population)

| | Placebo | AVP-786-28 | AVP-786-42.63 | All Patients |
|---|---|---|---|---|
| | (N = 210) | (N = 151) | (N = 160) | (N = 521) |
| Parameter | n (%) | n (%) | n (%) | n (%) |
| Patients with at least one n (%) | | | | |
| Fall | 9 (4.3) | 16 (10.6) | 10 (6.3) | 35 (6.7) |
| Severe fall | 1 (0.5) | 1 (0.7) | 0 | 2 (0.4) |
| Drag-related fall | 0 | 3 (2.0) | 0 | 3 (0.6) |
| Serious fall | 1 (0.5) | 3 (2.0) | 0 | 4 (0.8) |
| Drug-related serious fall | 0 | 1 (0.7) | 0 | 1 (0.2) |
| Patients discontinued treatment | 0 | 1 (0.7) | 0 | 1 (0.2) |
| because of a fall n (%) | | | | |

AE = adverse event;
MedDRA = Medical Dictionary for Regulatory Activities;
TEAE = treatment-emergent adverse event
Note:
Only TEAEs are included. A TEAE is defined as an AE where: first dose date ≤ AE start date ≤ date of last dose + 30.
A drug-related event is defined as an event assessed as Possibly Related or Related by the Investigator or where relationship records are missing.

### Sinus Bradycardia

Sinus bradycardia events includes TEAEs of sinus bradycardia and bradycardia. Four patients (0.8%) reported sinus bradycardia events (Table 89): 3 (1.4%), 1 (0.7%), and 0 patients in the placebo, AVP-786-28, and AVP-786-42.63 groups, respectively.

TABLE 89
Summary of Treatment-emergent Adverse Events - Sinus Bradycardia (Safety Population)

| | Placebo | AVP-786-28 | AVP-786-42.63 | All Patients |
|---|---|---|---|---|
| | (N = 210) | (N = 151) | (N = 160) | (N = 521) |
| Parameter | n (%) | n (%) | n (%) | n (%) |
| Patients with at least one n (%) | | | | |
| Sinus bradycardia event | 3 (1.4) | 1 (0.7) | 0 | 4 (0.8) |
| Severe sinus bradycardia event | 0 | 0 | 0 | 0 |
| Drug-related sinus bradycardia event | 2 (1.0) | 0 | 0 | 2 (0.4) |
| Serious sinus brady cardia event | 0 | 1 (0.7) | 0 | 1 (0.2) |
| Drug-related serious sinus bradycardia event | 0 | 0 | 0 | 0 |
| Sinus bradycardia event leading to | 0 | 0 | 0 | 0 |
| discontinuation of treatment | | | | |

AE = adverse event;
MedDRA = Medical Dictionary for Regulatory Activities;
TEAE = treatment-emergent adverse event
Note:
Sinus bradycardia events include sinus bradycardia and bradycardia.
Only TEAEs are included. A TEAE is defined as an AE where: first dose date ≤ AE start date ≤ date of last dose + 30.
A drug-related event is defined as an event assessed as Possibly Related or Related by the Investigator or where relationship records are missing.

### Rash

Rash events include TEAEs of rash and dermatitis contact. A total of 4 rash events were reported; 2 patients each in the placebo and AVP-78642.63 groups (1.0% and 1.3%, respectively; Table 90). All events of rash were considered mild in severity, none were reported as SAEs, and none led to treatment discontinuation.

TABLE 90
Summary of Treatment-emergent Adverse Events - Rash (Safety Population)

| | Placebo | AVP-786-28 | AVP-786-42.63 | All Patients |
|---|---|---|---|---|
| | (N = 210) | (N = 151) | (N = 160) | (N = 521) |
| Parameter | n (%) | n (%) | n (%) | n (%) |
| Patients with at least one n (%) | | | | |
| Rash event | 2 (1.0) | 0 | 2 (1.3) | 4 (0.8) |
| Severe rash event | 0 | 0 | 0 | 0 |
| Drug-related rash event | 0 | 0 | 1 (0.6) | 1 (0.2) |
| Serious rash event | 0 | 0 | 0 | 0 |
| Drug-related serious rash event | 0 | 0 | 0 | 0 |
| Rash event leading to discontinuation | 0 | 0 | 0 | 0 |
| of treatment | | | | |

AE = adverse event;
MedDRA = Medical Dictionary for Regulatory Activities;
TEAE = treatment-emergent adverse event
Note:
Rash events include TEAEs of rash and dermatitis contact.
Only TEAEs are included. A TEAE is defined as an AE where: first dose date ≤ AE start date ≤ date of last dose + 30.
A drug-related event is defined as an event assessed as Possibly Related or Related by the Investigator or where relationship records are missing.

Thrombocytopenia

No patients treated with AVP-786 reported TEAEs of thrombocytopenia, and 1 patient treated with placebo reported a TEAE of thrombocytopenia. The thrombocytopenia event was mild, nonserious, and considered not related to treatment; the dose of study drug was not changed due to the TEAE, and the event was ongoing at the end of study. The patient's (Patient 255-008) platelet count had decreased from a Baseline value of 129×10⁹/L (normal range 150450×10⁹/L) to 89×10⁹/L at Week 3 (it was 136×10⁹/L at retest, 2 days later). The patient's platelet count was 112×10⁹/L at Week 6 and 80×10⁹/L at retest, 6 days later, at which time the TEAE began. The patient completed the study, and platelets remained ≤95×10⁹/L for the remainder of the study.

On hematology laboratory tests results, 4 (2.0/6) patients in the placebo group and 1 (0.7%) patient in the AVP-786-28 group met potentially clinically significant (PCS) criterion for low platelets. One patient who met PCS criterion for low platelets in the placebo group was the same patient who reported a TEAE of thrombocytopenia. The percentage of patients with shifts in platelets from normal to low were 2.4%, 2.1%, and 1.3% for the placebo, AVP-786-28, and AVP-786-42.63 groups, respectively.

Serotonin Syndrome

No patients reported TEAEs of serotonin syndrome.

### 5.3. Clinical Laboratory Evaluation

### 5.3.1. Evaluation of Each Laboratory Parameter

In general, there was no evidence of clinically meaningful changes from Baseline in mean values of chemistry or hematology parameters, or in quantitative measures of urinalysis in any treatment group.

### 5.3.1.1. Laboratory Values Over Time

The proportion of patients with shifts in chemistry and hematology values from either low, normal, or high at Baseline to low, normal, or high at the end of treatment are presented by visit.

### 5.3.1.1.1. Chemistry

Overall, a small percentage of patients had shifts (low, normal, or high, relative to the normal range) from Baseline to end of treatment in chemistry parameters. The only chemistry parameters for which >10% of patients in any treatment group were both normal at Baseline and high at end of treatment were cholesterol (8.2%, 11.0%, and 11.4% in the placebo, AVP-786-28, and AVP-786-42.63 groups, respectively) and triglycerides (11.6%, 14.4%, and 11.4%, respectively).

### 5.3.1.1.2. Hematology

Overall, a small percentage of patients had shifts (low, normal, or high, relative to the normal range) from Baseline to end of treatment in hematology parameters. For no hematology parameters were >10% of patients in any treatment group both normal at Baseline and low at end of treatment.

### 5.3.1.1.3. Urinalysis

There were no clinically meaningful changes in urinalysis measures. There were a number of shifts out of the normal range for specific gravity, but they did not appear to be related to treatment group.

### 5.3.2. Individual Potentially Clinically Significant Abnormalities

Overall, a low percentage of patients met PCS criteria for chemistry or hematology parameters across all treatment groups. Table 91 summarizes the parameters where PCS criteria was met for ≥5% patients in any treatment group.

The proportion of patients with PCS abnormalities in chemistry or hematology parameters was generally similar among the treatment groups. A higher proportion of patients in the AVP-786-42.63 group met the PCS criteria compared with the placebo group for elevated potassium (7.6% vs 3.9%, respectively) and elevated eosinophils/leukocytes (8.9% vs 5.8%, respectively). A higher proportion of patients in the AVP-786-28 group met the PCS criteria compared with the placebo group for elevated glucose (15.8% vs 11.6%, respectively), elevated triglycerides (13.7% vs 11.1%, respectively), and elevated potassium (5.5% vs 3.9%, respectively).

TABLE 91
Most Common (≥5% Patients in Any Treatment Group) Potentially Clinically Significant
Postbaseline Chemistry and Hematology Laboratory Abnormalities (Safety Population)

| | | | Placebo | | | AVP-786-28 | | | AVP-786-42.63 | | | All Patients | | |
|---|---|---|---|---|---|---|---|---|---|---|---|---|---|---|
| | | | (N = 210) | | | (N = 151) | | | (N = 160) | | | (N = 521) | | |
| Parameter | PCS Criterion | | N | n (%) | | N | n (%) | | N | n (%) | | N | n (%) | |
| Blood Urea Nitrogen (mmol/L) | ≥10.71 | mmol/L | 207 | 29 | (14.0) | 146 | 18 | (12.3) | 158 | 12 | (7.6) | 511 | 59 | (11.5) |
| Creatinine (μmol/L) | >132.6 | μmol/L | 207 | 16 | (7.7) | 146 | 5 | (3.4) | 158 | 4 | (2.5) | 511 | 25 | (4.9) |
| Gamma-glutamyl Transferase (U/L) | ≥60 | U/L | 207 | 19 | (9.2) | 146 | 13 | (8.9) | 158 | 8 | (5.1) | 511 | 40 | (7.8) |
| Glucose (mmol/L) | ≥11.1 | mmol/L | 207 | 24 | (11.6) | 146 | 23 | (15.8) | 158 | 14 | (8.9) | 511 | 61 | (11.9) |
| Potassium (mmol/L) | ≥5.5 | mmol/L | 207 | 8 | (3.9) | 146 | 8 | (5.5) | 158 | 12 | (7.6) | 511 | 28 | (5.5) |
| Triglycerides (mmol/L) | >3.39 | mmol/L | 207 | 23 | (11.1) | 146 | 20 | (13.7) | 158 | 16 | (10.1) | 511 | 59 | (11.5) |

| | | | | | | | | | | | | | |
|---|---|---|---|---|---|---|---|---|---|---|---|---|---|
| Eosinophils/Leukocytes (%) | ≥10% | 206 | 12 | (5.8) | 146 | 10 | (6.8) | 158 | 14 | (8.9) | 510 | 36 | (7.1) |
| Hematocrit (%) | >0.5 proportion of 1.0 | 206 | 11 | (5.3) | 146 | 5 | (3.4) | 158 | 9 | (5.7) | 510 | 25 | (4.9) |

PCS = potentially clinically significant

### 5.4. Vital Signs, Physical Findings, and Other Observations Related to Safety

### 5.4.1. Electrocardiograms

In general, there was no evidence of clinically meaningful mean changes in any ECG parameter for any treatment group between or within visits. Mean and median changes for QTcF were within ±2% across visits and within visit for each group. The mean (SD) change from Baseline to Week 12 were 2.6 (12.7), 3.4 (14.3), and 2.7 (11.8) msec for the placebo, AVP-786-28, and AVP-786-42.63 groups, respectively. The mean changes from predose to postdose on Day 1 were 1.7 (13.5), 1.2 (12.9), and 3.4 (11.6) msec, respectively.

A small number of patients met the PCS criteria of QTcF>500 msec (males and females combined) or increase in QTcF≥60 msec compared to Baseline in the placebo, AVP-786-28, and AVP-786-42.63 groups (QTcF>500: 1 [0.5%], 1 [0.7%], and 0, respectively; increase in QTcF>60 msec: 0, 1 [0.7%], and 0, respectively).

Of the TEAEs that were ECG or cardiac rhythm abnormalities, atrial fibrillation (1 patient [0.5%], 2 [1.3%], and 0% in the placebo, AVP-786-28, and AVP-786-42.63 groups, respectively) and sinus bradycardia (2 [1.0%], 0%, and 0%, respectively were experienced by more than 1 patient in a single group.

TABLE 92
Overall Potentially Clinically Significant Postbaseline ECG Abnormalities (Safety Population)

| | | | Placebo | | | AVP-786-28 | | | AVP-786-42.63 | | | All Patients | | |
|---|---|---|---|---|---|---|---|---|---|---|---|---|---|---|
| | | | (N = 210) | | | (N = 151) | | | (N = 160) | | | (N = 521) | | |
| Parameter | PCS Criterion | | N | n (%) | | N | n (%) | | N | n (%) | | N | n (%) | |
| PR Interval Value | >200 to ≤220 msec | | 202 | 26 | (12.9) | 148 | 17 | (11.5) | 158 | 19 | (12.0) | 508 | 62 | (12.2) |
| (males and females) | >220 to ≤250 msec | | 202 | 19 | (9.4) | 148 | 13 | (8.8) | 158 | 9 | (5.7) | 508 | 41 | (8.1) |
| | >250 | msec | 202 | 2 | (1.0) | 148 | 2 | (1.4) | 158 | 2 | (1.3) | 508 | 6 | (1.2) |
| QTcF Value (males) | >450 to ≤480 msec | | 87 | 7 | (8.0) | 65 | 8 | (12.3) | 63 | 8 | (12.7) | 215 | 23 | (10.7) |
| | >480 to ≤500 msec | | 87 | 1 | (1.1) | 65 | 0 | | 63 | 0 | | 215 | 1 | (0.5) |
| | >500 | msec | 87 | 0 | | 65 | 1 | (1.5) | 63 | 0 | | 215 | 1 | (0.5) |
| QTcF Value (females) | >470 to ≤485 msec | | 115 | 5 | (4.3) | 83 | 3 | (3.6) | 95 | 2 | (2.1) | 293 | 10 | (3.4) |
| | >485 to ≤500 msec | | 115 | 2 | (1.7) | 83 | 1 | (1.2) | 95 | 0 | | 293 | 3 | (1.0) |
| | >500 | msec | 115 | 1 | (0.9) | 83 | 0 | | 95 | 0 | | 293 | 1 | (0.3) |
| QTcF Change from Baseline | ≥30 | msec | 199 | 13 | (6.5) | 146 | 18 | (12.3) | 158 | 12 | (7.6) | 503 | 43 | (8.5) |

| | | | | | | | | | | | | |
|---|---|---|---|---|---|---|---|---|---|---|---|---|
| (male and females) | ≥60 | msec | 199 | 0 | 146 | 1 | (0.7) | 158 | 0 | 503 | 1 | (0.2) |

ECG = electrocardiogram;
QTcF = QT corrected using Fridericia's method
Note:
Baseline is defined as the last non-missing assessment prior to first dose of study drug.

TABLE 93
Electrocardiogram and Cardiac Rhythm Abnormalities Reported
as Treatment-Emergent Adverse Events (Safety Population)

| | Placebo | AVP-786-28 | AVP-786-42.63 | All Patients |
|---|---|---|---|---|
| System Organ Class (SOC) | (N = 210) | (N = 151) | (N = 160) | (N = 521) |
| Preferred Term (PT) | n (%) | n (%) | n (%) | n (%) |
| Cardiac disorders | | | | |
| Atrial fibrillation | 1 (0.5) | 2 (1.3) | 0 | 3 (0.6) |
| Supraventricular extrasystoles | 1 (0.5) | 0 | 1 (0.6) | 2 (0.4) |
| Bradycardia | 1 (0.5) | 1 (0.7) | 0 | 2 (0.4) |
| Sinus bradycardia | 2 (1.0) | 0 | 0 | 2 (0.4) |
| Atrioventricular block first degree | 0 | 0 | 1 (0.6) | 1 (0.2) |
| Bundle branch block left | 0 | 0 | 1 (0.6) | 1 (0.2) |
| Investigations | | | | |
| Electrocardiogram QT prolonged | 1 (0.5) | 1 (0.7) | 0 | 2 (0.4) |
| Electrocardiogram T wave inversion | 0 | 0 | 1 (0.6) | 1 (0.2) |
| Electrocardiogram abnormal | 1 (0.5) | 0 | 0 | 1 (0.2) |

MedDRA = Medical Dictionary for Regulatory Activities; TEAE = treatment-emergent adverse event.
Note:
Adverse events ate coded using MedDRA version 18.1
If a patient has more than one TEAE that codes to the same MedDRA category, the patient is counted only once in the MedDRA category.

### 5.4.2. Vital Signs

There were no notable mean or median changes from Baseline to any postbaseline visit in the standing or supine position for systolic BP, diastolic BP, HR, respiration rate, or temperature.

There were no notable mean or median changes from supine to standing at any postbaseline visit. HR increases upon standing were up to 10% but similar in all treatment groups.

The proportions of patients experiencing PCS vital signs abnormalities were low and similar in the placebo, AVP-786-28, and AVP-786-42.63 groups.

The proportion of patients with PCS orthostatic hypotension was high in all groups (17.8%, 22.5%, and 21.5% in the placebo, AVP-786-28, and AVP-786-42.63 groups, respectively). Overall, 14 patients (2.7%) had TEAEs of dizziness; by treatment group, the proportions of patients with TEAEs of dizziness were 1.9%, 2.0%, and 4.4%, respectively. Three patients (0.6%) had TEAEs of syncope (all in the AVP-786-28 group). Five patients (1.0%) had TEAEs of hypotension (0.5%, 2.0%, and 0.6%, respectively), and 1 (0.5%) had a TEAE of orthostatic hypotension (in the placebo group). Falls are presented elsewhere herein.

TABLE 94
Potentially Clinically Significant Postbaseline Vital Sign Abnormalities (Safety Population)

| | | Placebo | | | AVP-786-28 | | | AVP-786-42.63 | | | All patients | | |
|---|---|---|---|---|---|---|---|---|---|---|---|---|---|
| | | (N = 210) | | | (N = 151) | | | (N = 160) | | | (N = 521) | | |
| Parameter | PCS Criterion | N | n (%) | | N | n (%) | | N | n (%) | | N | n (%) | |
| Systolic Blood | ≤90 and ≥20 decrease from Baseline | 210 | 6 | (2.9) | 150 | 2 | (1.3) | 158 | 3 | (1.9) | 518 | 11 | (2.1) |
| Pressure (SBP) | >180 and ≥20 increase from Baseline | 210 | 2 | (1.0) | 150 | 1 | (0.7) | 158 | 0 | | 518 | 3 | (0.6) |
| Diastolic Blood | ≤50 and ≥15 decrease from Baseline | 210 | 8 | (3.8) | 150 | 2 | (1.3) | 158 | 2 | (1.3) | 518 | 12 | (2.3) |
| Pressure (DBP) | ≥105 and ≥15 increase from Baseline | 210 | 0 | | 150 | 1 | (0.7) | 158 | 2 | (1.3) | 518 | 3 | (0.6) |
| Heart Rate (HR) | ≤50 and ≥15 decrease from Baseline | 210 | 2 | (1.0) | 150 | 0 | | 158 | 3 | (1.9) | 518 | 5 | (1.0) |
| | ≥120 and ≥15 increase from Baseline | 210 | 1 | (0.5) | 150 | 0 | | 158 | 0 | | 518 | 1 | (0.2) |
| SBP and HR | SBP ≥10 and HR ≥5 increase from | 210 | 62 | (29.5) | 150 | 44 | (29.3) | 158 | 39 | (24.7) | 518 | 145 | (28.0) |
| | Baseline | | | | | | | | | | | | |
| DBP and HR | DBP ≥5 and HR ≥5 increase from | 210 | 83 | (39.5) | 150 | 54 | (36.0) | 158 | 42 | (26.6) | 518 | 179 | (34.6) |
| | Baseline | | | | | | | | | | | | |

PCS = potentially clinically significant
Note:
Baseline is defined as the last non-missing assessment prior to first dose of study drug.
Criteria involving multiple parameters will be included only if they take place at the same visit.

TABLE 95
Potentially Clinically Significant Orthostatic Hypotension and Postural Tachycardia (Safety Population)

| | Placebo | | | AVP-786-28 | | | AVP-786-42.63 | | | All Patients | | |
|---|---|---|---|---|---|---|---|---|---|---|---|---|
| | (N = 210) | | | (N = 151) | | | (N = 160) | | | (N = 521) | | |
| Potentially Clinically Significant Criterion | N | n (%) | | N | n (%) | | N | n (%) | | N | n (%) | |
| Orthostatic hypotension - decrease of ≥20 mmHg in SBP | 197 | 35 | (17.8) | 142 | 32 | (22.5) | 158 | 34 | (21.5) | 497 | 101 | (20.3) |
| or ≥10 mmHg in DBP change from supine to standing | | | | | | | | | | | | |
| Postural tachycardia - increase of ≥30 bpm in HR | 197 | 2 | (1.0) | 142 | 1 | (0.7) | 158 | 2 | (1.3) | 497 | 5 | (1.0) |
| change from supine to standing or standing HR ≥120 bpm | | | | | | | | | | | | |

DBP = diastolic blood pressure;
HR = heart rate;
SBP = systolic blood pressure
Note:
Baseline is defined as the last non-missing assessment prior to first dose of study drug.
Criteria involving multiple parameters will be included only if they take place at the same visit.

### 5.4.3. Sheehan Suicidality Tracking Scale

There was no evidence of an increase in suicidal behavior or ideation in any treatment group based on postbaseline assessments of the S-STS.

### 5.4.4. Mini Mental State Examinations

There was no evidence of clinically significant mean or median change in cognition in any treatment group, as measured by the MMSE Total scores. The mean (SD) changes from Baseline to Week 12 were 0.4 (3.1), 0.8 (2.9), and 1.1 (2.5) for the placebo, AVP-786-28, and AVP-786-42.63 groups, respectively.

### 5.4.5. Timed Up and Go Test

There was no evidence of clinically significant mean or median changes in the TUG test in any group.

### 5.4.6. Epworth Sleepiness Scale

The mean (SD) changes from Baseline to Week 12 were −0.4 (3.1), 0.0 (4.3), and −0.8 (4.1) for the placebo, AVP-786-28, and AVP-786-42.63 groups, respectively. TEAEs related to sleepiness were somnolence (1.0%, 2.6%, and 6.9%, respectively), fatigue (1.0%, 0.7%, and 0.6%, respectively) lethargy (0%, 1.3%, and 0%, respectively), and sedation (0.5%, 0%, and 0%, respectively).

### 5.5. Safety Conclusions

The incidences of TEAEs were similar in the placebo, AVP-786-28, and AVP-786-42.63 groups: 44.8%, 48.3%, and 46.3%, respectively.

The most frequently reported TEAEs (≥2% of patients in any treatment group) that occurred in a higher percentage of patients in the AVP-786 groups were:
Fall (4.3%, 10.6%, and 6.3% for the placebo, AVP-786-28, and AVP-786-42.63 groups, respectively)
Urinary tract infection (5.2%, 7.3%, 2.5%, respectively)
Somnolence (1.0%, 2.6%, and 6.9%, respectively)
Dizziness (1.9%, 2.0%, and 4.4%, respectively)
Hot flush (0%, 2.0%, and 0%, respectively)
Syncope (0%, 2.0%, and 0%, respectively)
Agitation was reported in a higher percentage of patients in the placebo group compared with the AVP-786-28 and AVP-786-42.63 groups (5.7%, 1.3%, and 3.1%, respectively).

Patients in the AVP-786-28 and AVP-786-42.63 groups had a higher incidence of drug-related TEAEs compared with the placebo group (6.2%, 12.6%, and 15.0% for the placebo, AVP-786-28, and AVP-786-42.63 groups, respectively). The most frequently reported drug-related TEAEs (? 2% of patients in any treatment group) were somnolence (1.0%, 1.3%, and 5.6%, respectively), dizziness (0%, 0.7%, and 2.5%, respectively), headache (0.5%, 2.0%, and 0.6%, respectively), and fall (0%, 2.0%, and 0%, respectively).

A higher percentage of patients in the AVP-786-28 and AVP-786-42.63 groups discontinued treatment due to TEAEs compared with the placebo group (1.0%, 6.6%, and 3.8% patients in the placebo, AVP-786-28, and AVP-786-42.63 groups, respectively). No single TEAE led to discontinuation of more than 1 patient in any treatment group.

Patients in the AVP-786-28 group had a higher incidence of SAEs compared with the placebo and AVP-786-42.63 groups (4.8%, 9.9%, and 5.0%, in the placebo, AVP-786-28, and AVP-786-42.63 groups, respectively). The most frequently reported SAEs (≥2 patients in any treatment group) that occurred in a higher number of patients in the AVP-786 groups were:
Pneumonia (0.5%, 2.0%, and 0.6% in the placebo, AVP-786-28, and AVP-786-42.63 groups, respectively)
Fall (0.5%, 2.0%, and 0%, respectively)
Mental status changes (0%, 0.7%, and 1.3%, respectively)
Syncope (0%, 2.0%, and 0%, respectively)

Three patients (0.6%) died during the study, all in the AVP-786-28 group. None of the deaths were considered related to study drug by the Investigator.

The TEAEs of interest for AVP-786 were events of fall, sinus bradycardia, rash, thrombocytopenia, and serotonin syndrome.
Fall was the most frequently reported TEAE across all treatment groups; 6.7% patients reported TEAEs of fall (4.3%, 10.6%, and 6.3% for the placebo, AVP-786-28, and AVP-786-42.63 groups, respectively). TEAEs of fall were generally mild to moderate in severity. Few falls were reported as SAEs (0.8%), led to discontinuation of study drug (0.2%), or were considered related to study drug (0.6%).
For the remaining TEAEs of interest, low numbers of patients reported these types of events. Four patients reported sinus bradycardia events (1.4%, 0.7%, and 0% for the placebo, AVP-786-28, and AVP-786-42.63 groups, respectively). Four patients reported rash events (1.0%, 0%, and 1.3%, respectively). One patient treated with placebo reported a TEAE of thrombocytopenia. No patients reported TEAEs of serotonin syndrome.

No additional safety risks were identified by clinical laboratory tests, ECGs, or vital signs. No increased risk of cognitive worsening or suicidality was observed in patients treated with AVP-786 compared with patients treated with placebo.

### 6. Discussion and Overall Conclusions

Agitation and/or aggression are estimated to affect up to approximately 80% of patients with dementia, and Alzheimer's disease is the most common form of dementia. Agitation in dementia has a significant negative impact on functional ability, QOL, caregiver burden, institutionalization, health care expenses, and mortality. AVP-786 is expected to reduce agitation in Alzheimer's dementia.

This was a Phase 3, multicenter, randomized, double-blind, placebo-controlled study to assess the efficacy, safety, and tolerability of AVP-786 for the treatment of patients with clinically significant, moderate/severe agitation associated with dementia of the Alzheimer's type.

A total of 522 patients were randomized to treatment, and 519 of them were included in the mITT Population, with 210, 150, and 159 mITT patients in the placebo, AVP-786-28, and AVP-786-42.63 groups, respectively. The groups in the mITT Population were well balanced with regard to sex, race, ethnicity, and age. Most patients completed the study (Section 3.1).

Although there were no significant differences between the AVP-786 and placebo groups on the primary endpoint, the CMAI Total score, there were numeric improvements in favor of AVP-786-42.63 compared with placebo (treatment difference−2.0 [−5.0 to 1.0]; p=0.200).

Additional efficacy endpoints, which were significant (nominal significance level, p<0.05) for AVP-786-42.63 compared to placebo, included:
NPI—Irritability Domain score
NPI Total score

### Pharmacokinetics

There did not appear to be any significant changes in d6-DM, d3-DX, d3-3-MM, and Q plasma concentrations from Week 6 to Week 12. Exposures to d6-DM and d3-DX increased with the increase in d6-DM dose of AVP-786 and is consistent with the patient genotypes for CYP2D6.

### Safety

Treatment with AVP-786-28 and AVP-786-42.63 was generally well tolerated during the study. The incidences of TEAEs were similar in the placebo, AVP-786-28, and AVP-786-42.63 groups (44.8%, 48.3%, and 46.3%, respectively). The most frequently reported TEAEs (≥2%) that occurred in a higher percentage of patients in an AVP-786 treatment group were fall, urinary tract infection, somnolence, dizziness, hot flush, and syncope; however, few were considered related to study drug or led to treatment discontinuation.

Overall, the incidence of discontinuations due to TEAEs (3.5%) and the incidence of SAEs (6.3%) and was low for a 12-week study in an elderly patient population. A higher percentage of patients in the AVP-786-28 and AVP-786-42.63 groups discontinued treatment due to TEAEs compared with the placebo group (1.0%, 6.6%, and 3.8% patients in the placebo, AVP-786-28, and AVP-786-42.63 groups, respectively), and the incidence of SAEs was higher on AVP-786-28 than either of the other treatment arms (4.8%, 9.9%, and 5.0%, respectively). No single TEAE led to discontinuation of more than 1 patient in any treatment group.

The most frequently reported SAEs were pneumonia, fall, mental status changes, and syncope. No SAE was reported by more than 3 patients in any treatment group.

Three patients (0.6%) died during the study, all in the AVP-786-28 group. None of the deaths were considered related to study drug by the Investigator.

The TEAEs of interest for AVP-786 were events of fall, sinus bradycardia, rash, thrombocytopenia, and serotonin syndrome. Fall was the most frequently reported TEAE across all treatment groups. Thirty-five (6.7%) patients reported TEAEs of fall (4.3%, 10.6%, and 6.3% for the placebo, AVP-786-28, and AVP-786-42.63 groups, respectively). These TEAEs were generally mild to moderate in severity, and few were reported as SAEs, led to discontinuation of study drug, or were considered related to study drug. Other TEAEs of interest were uncommon (<1.0% overall), were rarely severe or serious, and were rarely the cause of discontinuation.

No additional clinically significant safety risks were identified by clinical laboratory tests, ECGs, or vital signs. No increased risk of cognitive decline or suicidality was observed in patients treated with AVP-786 compared with patients treated with placebo. Overall Conclusions:

There were no statistically significant differences between the AVP-786 and placebo groups in the change from Baseline at Week 12 in the CMAI Total score; however, there were numeric improvements in favor of AVP-786-42.63 compared to placebo.

Treatment with AVP 786 was safe and generally well tolerated at both dose levels. The incidences of TEAEs, drug-related TEAEs, SAEs, and discontinuations due to TEAEs were similar in the placebo and AVP 786 42.63 groups, but higher in the AVP 786 28 group. No additional safety risks were identified by clinical laboratory tests, ECGs, or vital signs. No increased risk of cognitive worsening or suicidality was observed in patients treated with AVP-786 compared with patients treated with placebo.

1-119. (canceled)

120. A method of treating agitation associated with Alzheimer's disease in a patient that has been diagnosed as having Alzheimer's disease, wherein the patient has an MMSE score from 8 to 20,
wherein the method comprises:
a) determining the Cohen-Mansfield agitation inventory (CMAI) total score in the patient;
and
b) administering to the patient effective amounts of d6-DM and quinidine sulfate.

121. The method of claim 120, wherein the effective amount of d6-DM is 28 mg.

122. The method of claim 120, wherein the effective amount of d6-DM is 42.63 mg.

123. The method of claim 120, wherein administration of effective amounts of d6-DM and quinidine sulfate is twice daily.

124. The method of claim 120, wherein following administration of effective amounts of d6-DM and quinidine sulfate for one week, the CMAI total score is less than the CMAI total score prior to the administering step in the patient by about 8 points to about 10 points, such as about 9 points to about 10 points, such as 8.9 points to 9.8 points.

125. The method of claim 120, wherein following administration of effective amounts of d6-DM and quinidine sulfate for two weeks, the CMAI total score is less than the CMAI total score prior to the administering step in the patient by about 12 points to about 13 points, such as 12.3 points to 12.9 points.

126. The method of claim 120, wherein following administration of effective amounts of d6-DM and quinidine sulfate for three weeks, the CMAI total score is less than the CMAI total score prior to the administering step in the patient by about 14 points to about 17 points, such as about 14 points to about 16 points, such as 14.6 points to 16.2 points.

127. The method of claim 120, wherein following administration of effective amounts of d6-DM and quinidine sulfate for six weeks, the CMAI total score is less than the CMAI total score prior to the administering step in the patient by about 15 points to about 17 points, such as about 15 points to about 16 points, such as 15.2 points to 16.1 points.

128. The method of claim 120, wherein following administration of effective amounts of d6-DM and quinidine sulfate for nine weeks, the CMAI total score is less than the CMAI total score prior to the administering step in the patient by about 17 points to about 19 points, such as 17.2 points to 18.7 points.

129. The method of claim 120, wherein following administration of effective amounts of d6-DM and quinidine sulfate for twelve weeks, the CMAI total score is less than the CMAI total score prior to the administering step in the patient by about 14 points to about 22 points, such as 14.3 points to 21.6 points.

130. The method of claim 120, wherein the method comprises determining that the Cohen-Mansfield agitation inventory (CMAI) total score in the patient is from about 76 to about 79 prior to the administering step.

131. The method of claim 120, wherein the method comprises determining that the Cohen-Mansfield agitation inventory (CMAI) total score in the patient is from 76.6 to 78.8 prior to the administering step.

132. The method of claim 122, wherein following administration of effective amounts of d6-DM and quinidine sulfate for one week, the CMAI total score is less than the CMAI total score prior to the administering step in the patient by about 8 points to about 9 points, such as about 9 points, such as 8.9 points.

133. The method of claim 122, wherein following administration of effective amounts of d6-DM and quinidine sulfate for two weeks, the CMAI total score is less than the CMAI total score prior to the administering step in the patient by about 12 points to about 13 points, such as about 12 points, such as 12.3 points.

134. The method of claim 122, wherein following administration of effective amounts of d6-DM and quinidine sulfate for three weeks, the CMAI total score is less than the CMAI total score prior to the administering step in the patient by about 14 points to about 15 points, such as about 15 points, such as 14.6 points.

135. The method of claim 122, wherein following administration of effective amounts of d6-DM and quinidine sulfate for six weeks, the CMAI total score is less than the CMAI total score prior to the administering step in the patient by about 16 points to about 17 points, such as about 16 points, such as 16.1 points.

136. The method of claim 122, wherein following administration of effective amounts of d6-DM and quinidine sulfate for nine weeks, the CMAI total score is less than the CMAI total score prior to the administering step in the patient by about 18 points to about 19 points, such as about 19 points, such as 18.7 points.

137. The method of claim 122, wherein following administration of effective amounts of d6-DM and quinidine sulfate for twelve weeks, the CMAI total score is less than the CMAI total score prior to the administering step in the patient by about 21 points to about 22 points, such as about 22 points, such as 21.6 points.

138. The method of claim 122, wherein the method comprises determining that the Cohen-Mansfield agitation inventory (CMAI) total score in the patient is from about 78 to about 79, such as about 79, such as 78.8, prior to the administering step.

139. The method of claim 120, wherein the d6-DM comprises a deuterated [d6]-dextromethorphan hydrobromide hydrate.

140. The method of claim 139, wherein the d6-DM comprises [d6]-dextromethorphan hydrobromide monohydrate.

141. The method of claim 120, wherein the patient has been assessed as having a CMAI aggressive behavior score of greater than or equal to 15.

142. The method of claim 141, wherein the patient has been assessed as having a CMAI aggressive behavior score of 15 to 40.

143. The method of claim 120, wherein the patient has been assessed to have a score of 2 to 7 for at least one CMAI aggressive behavior item selected from the group consisting of;
1) hitting (including self);
2) kicking;
3) grabbing onto people;
4) pushing;
5) throwing things;
6) biting;
7) scratching;
8) spitting;
9) hurting self or others;
10) tearing things or destroying property;
11) screaming; and
12) cursing or verbal aggression.

144. The method of claim 143, wherein the patient has been assessed to have a score of 2 to 5 for the at least one CMAI aggressive behavior item.
